(19) United States
(12) Patent Application Publication
(10) Pub. No.: US 2012/0079627 A1
(43) Pub. Date: Mar. 29, 2012

(54) PLANT GENE REGULATORY ELEMENTS

(75) Inventors: Srinivas Gampala, Manhattan, KS (US); Prasanna Kankanala, Manhattan, KS (US); David Lee, Arlington, VA (US); Emily Puckett, Arlington, VA (US); Ramesh Nair, Manhattan, KS (US); Forrest Chumley, Manhattan, KS (US)

(73) Assignee: EDENSPACE SYSTEMS CORPORATION, Manhattan, KS (US)

(21) Appl. No.: 13/375,128

(22) PCT Filed: Jun. 1, 2010
(86) PCT No.: PCT/US2010/036945

§ 371 Date: Nov. 29, 2011

Related U.S. Application Data
(60) Provisional application No. 61/182,467, filed on May 29, 2009.

(51) Int. Cl. A01H 5/00; A01H 5/10; C12N 15/82; C12N 15/113; C12N 15/63

(52) U.S. Cl. 800/300; 536/24.1; 435/320.1; 800/298; 800/303; 800/301; 800/302; 800/320; 800/320.1; 800/320.2; 800/320.3; 800/314; 800/306; 800/317.2; 800/312; 800/322; 800/317.3; 800/317.4

(57) ABSTRACT

Nucleic acids, vectors, and expression vectors comprising novel plant gene regulatory elements from poplar that can drive heterologous gene expression in plants. Novel transgenic plants expressing heterologous genes under the control of novel gene regulatory elements.

### RELATED APPLICATION INFORMATION

The present application claims priority to and benefit of U.S. provisional patent application 61/182,467, filed on May 29, 2009, the contents of which are herein incorporated by reference in their entirety.

### SEQUENCE LISTING

The present specification makes reference to a Sequence Listing (submitted electronically as a .txt file named “SequenceListing.txt” on Jun. 1, 2010). The .txt file was generated on Jun. 1, 2010 and is 671 kb in size. The entire contents of the Sequence Listing are herein incorporated by reference.

### BACKGROUND

Plant gene expression is highly regulated in a tissue-specific and developmental stage-specific manner. Plant gene expression is also regulated in response to many external factors, including biotic and abiotic stress. Nucleotide sequences upstream of gene coding sequences, commonly known as promoters, precisely regulate when and where any particular gene is expressed. Promoters also control the extent of foreign gene expression in transgenic plants and hence are crucial in determining the levels to which a desirable gene can be expressed.

Over the last three decades, plant biologists have isolated and characterized several plant promoters that can drive heterologous transgene expression. These well-characterized promoters include CaMV 35S promoter (Odell et al. (1985) Nature. 313:810-812), Opine promoters (U.S. Pat. No. 5,955,646), the rice actin promoter (McElroy et al. (1991) Mol Gen Genet. 231:150-160), the maize ubiquitin promoter (Christensen et al. (1992) Plant Mol Biol. 18:675-89.), the maize ADH1 promoter (U.S. Pat. No. 5,001,060) and the Rubisco promoter (Outchkourov et al. (2003) Planta 216:1003-1012).

Many of the dicot promoters do not perform satisfactorily in monocots such as maize and other cereal crops or grasses. In general, dicot promoters do not require intron sequences downstream of the transcription initiation site to enhance gene expression in transgenic dicot plants, whereas the first intron downstream of monocot promoters often enhances gene expression in transgenic monocot plants (McElroy et al. (1991) Mol Gen Genet. 231:150-160 and Christensen et al. (1992) Plant Mol Biol. 18:675-89).

Functional assays have demonstrated that differences in required promoter elements of dicot and monocot promoters may be one of the reasons why dicot promoters do not necessarily work well in monocots and vice versa.

### SUMMARY

The present invention encompasses the recognition that while transgenic dicot plants containing multiple transgenes (stacked traits) are desirable, the ability to create such plants is limited by the availability of suitable promoters for each transgene. The present invention further encompasses the recognition that a collection of novel dicot promoters, with divergent DNA sequences and an optimal range of functional characteristics, would, among other things, facilitate creating of transgenic dicot plants.

In various aspects, provided are a collection of novel dicot gene regulatory elements (including promoters) from the poplar genome, as well as nucleic acids and vectors (including gene expression vectors) comprising such novel gene regulatory elements. In one aspect, transgenic plants expressing a heterologous gene under the control of novel dicot gene regulatory elements are provided. Novel gene regulatory elements of the invention may in some be embodiments be used in other plants, including other dicots, as well as monocots and multicotyledonous plants.

### BRIEF DESCRIPTION OF THE DRAWING

FIGS. 1A and 1B schematically illustrate particle bombardment expression vectors pUC18-GUSintron-NOS and pUC18-GUS-NOS respectively. These vectors contain a multiple cloning site (MCS), a GUS reporter gene with the catalase intron (GUSintron; FIG. 1A) or without the catalase intron (GUS; FIG. 1B), and the nopaline synthase terminator (NOS).

FIGS. 2A and 2B schematically illustrate particle bombardment expression vectors pUC18-PtP-GUSintron-NOS and pUC18-PtP-GUS-NOS respectively. These vectors contain various inventive poplar promoters (PtP), the GUS reporter gene with the catalase intron (GUSintron; FIG. 2A) or without the catalase intron (GUS; FIG. 2B), and the nopaline synthase terminator (NOS).

FIG. 3 shows GUS reporter gene expression driven by various inventive poplar promoters in poplar leaves. (Expression correlates with blue spots). CMPS, Cestrum Yellow Leaf Curling Virus promoter—short version; PtCal2, poplar calmodulin like-2 promoter; PtUbi2, poplar ubiquitin like-2 promoter, PtL5L2, poplar ribosomal protein L5 like-2 promoter; PtElf1a, poplar elongation factor 1a like-1 promoter.

FIG. 4 shows GUS reporter gene expression driven by inventive poplar promoter in poplar stem tissues. (Expression correlates with blue spots). CMPS, Cestrum Yellow Leaf Curling Virus promoter—short version; PtCal2, poplar calmodulin like-2 promoter; PtUbi2, poplar ubiquitin like-2 promoter, PtL5L2, poplar ribosomal protein L5 like-2 promoter; PtElf1a, poplar elongation factor 1a like-1 promoter.

FIGS. 5A and 5B schematically illustrate plant transformation binary vectors pED-MCS-GOI-NOS and pED-PtP-GOI-NOS respectively. pED-MCS-GOI-NOS contains a multi cloning site into which the various invenive poplar promoters (PtP) were cloned (FIG. 5B). ‘GOI’ refers to the gene of interest and ‘NOS’ refers to the nopaline synthase terminator. ‘LB’ indicates the T-DNA left border sequence and ‘RB’ indicates the T-DNA right border sequence.

FIG. 6 depicts results from an experiment evaluating β-glucan glucohydrolase expression driven by poplar promoter PtL5L2 of the present invention in comparison to that of the CMPS and 35S CMV promoters in five different transgenic events. Expression was measured by assaying glucan glucohydrolase enzyme activity on MUC substrate.

FIG. 7 depicts results from experiments evaluating GUS reporter gene expression driven by various inventive poplar promoters in stable poplar transgenic leaf (FIGS. 7A-F) and root (FIG. 7G-I) tissues. A non-transgenic poplar leaf was stained for GUS activity (FIG. 7J) as a negative control.

FIG. 8 depicts measured MUC activity levels for E1 endoglucanase gene driven by 35S, PtL5L2, PtUbi2 and PtPAL2 promoters in tobacco leaf infiltration experiments, along with a negative control (C—). Activity is normalized to the MUC activity (μmol hydrolyzed per μg protein) of the 35S:E1 construct.

FIG. 9 depicts results from experiments evaluating GUS reporter gene expression driven by various inventive poplar promoters in tobacco leaves infiltrated by Agrobacterium transformed with relevant expression vectors. FIG. 9A shows images of leaf samples transformed with GUS expression vectors under the control of a (C-IM), PtERD4 (pABC262), or PtSAM2 (pABC263) promoter. GUS expression correlates with overall light blue color in leaves expressing GUS under the control of PtERD4 and PtSAM2. FIG. 9B shows images of leaf samples transformed with GUS expression vectors under the control of a control (C-IM) or PtUbi2 (pABC267) promoter. GUS expression correlates with distinct blue color in leaves. The primary and secondary veins did not show any GUS staining and retained the green color.

### DEFINITIONS

Throughout the specification, several terms are employed that are defined in the following paragraphs.

As used herein, the terms “about” and “approximately”, in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the phrase “binary vector” refers to cloning vectors that are capable of replicating in both E. coli and Agrobacterium tumefaciens. In a binary vector system, two different plasmids are employed for generating transgenic plants. In many embodiments, the first plasmid is a small vector known as disarmed Ti plasmid has an origin of replication (ori) that permits the maintenance of the plasmid in a wide range of bacteria including E. coli and Agrobacterium. In many embodiments, the small vector contains foreign DNA in place of T-DNA, the left and right T-DNA borders (or at least the right T-border), markers for selection and maintenance in both E. coli and A. tumefaciens, and a selectable marker for plants. In many embodiments, the second plasmid is known as helper Ti plasmid, harbored in A. tumefaciens, which lacks the entire T-DNA region but contains an intact vir region essential for transfer of the T-DNA from Agrobacterium to plant cells.

As used herein, the phrase “cell wall-modifying enzyme polypeptide” refers to a polypeptide that modifies at least one component (e.g., xylans, xylan side chains, glucuronoarabinoxylans, xyloglucans, mixed-linkage glucans, pectins, pectates, rhamnogalacturonans, rhamnogalacturonan side chains, lignin, cellulose, mannans, galactans, arabinans, oligosaccharides derived from cell wall polysaccharides, and combinations thereof) or interaction (e.g., covalent linkage, ionic bond interaction, hydrogen bond interaction, and combinations thereof) in plant cell wall. In some embodiments, cell wall-modifying enzyme polypeptides have at least 50%, 60%, 70%, 80% or more overall sequence identity with a polypeptide whose amino acid sequence is set forth in Table 1 of co-pending U.S. patent application Ser. No. 12/476,247 (filed on Jun. 1, 2009), the contents of which are herein incorporated by reference in their entirety. Alternatively or additionally, in some embodiments, cell wall-modifying enzyme polypeptide shows at least 90%, 95%, 96%, 97%, 98%, 99%, or greater identity with at least one sequence element found in a polypeptide whose amino acid sequence is set forth in Table 1 of co-pending U.S. patent application Ser. No. 12/476,247, which sequence element is at least 4, 5, 6, 7, 8, 9, 10, 11, 12, 13, 14, 15, 16, 17, 18, 19, 20 or more amino acids long. In some embodiments, a provided cell wall-modifying enzyme polypeptide disrupts a linkage selected from the group consisting of hemicellulose-cellulose-lignin, hemicellulose-cellulose-pectin, hemicellulosediferululate-hemicellulose, hemicellulose-ferulate-lignin, mixed beta-D-glucan-cellulose, mixed-beta-D-glucan-hemicellulose, pectin-ferulate-lignin linkages, and combinations thereof.

As used herein, the term “construct”, when used in reference to a gene and/or nucleic acid, refers to a functional unit that allows expression of a gene of interest. Nucleic acid constructs typically comprise, in addition to the gene of interest (i.e., the heterologous gene whose expression is desired), a gene regulatory element capable of driving expression of the gene of interest (such as a promoter) and a terminator (also known as a stop signal), both of which are operably linked to the gene of interest. In some embodiments, constructs comprise additional sequences, e.g. marker genes that are also accompanied by a gene regulatory element (such as a promoter) and a terminator. In many embodiments, the sequences for each of the elements in the construct do not exist in this combination and arrangement in nature and/or are arranged and/or combined by the hand of man.

As used herein, the phrase “externally applied”, when used to describe enzyme polypeptides used in the processing of biomass, refers to enzyme polypeptides that are not produced by the organism whose biomass is being processed. “Externally applied” enzyme polypeptides as used herein does not include enzyme polypeptides that are expressed (whether endogenously or transgenically) by the organism (e.g., plant) from which the biomass is obtained.

As used herein, the term “extract”, when used as noun, refers to a preparation from a biological material (such as lignocellulosic biomass) in which a substantial portion of proteins are in solution. In some embodiments of the invention, the extract is a crude extract, e.g., an extract that is prepared by disrupting cells such that proteins are solubilized and optionally removing debris, but not performing further purification steps. In some embodiments of the invention, the extract is further purified in that certain substances, molecules, or combinations thereof are removed.

As used herein, the term “gene” refers to a discrete nucleic acid sequence responsible for a discrete cellular product and/or performing one or more intracellular or extracellular functions. More specifically, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. The gene and regulatory sequences may be derived from the same natural source, or may be heterologous to one another. The definition can also include nucleic acids that do not encode proteins but rather provide templates for transcription of functional RNA molecules such as tRNAs, rRNAs, etc. Alternatively, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA) or a protein produced by translation of an mRNA. Gene products also include RNAs that are modified by processes such as capping, polyadenylation, methylation, and editing, proteins post-translationally modified, and proteins modified by, for example, methylation, acetylation, phosphorylation, ubiquitination, ADP ribosylation, myristilation, and glycosylation.

The terms “genetically modified” and “transgenic” are used herein interchangeably. A transgenic or genetically modified organism is one that has a genetic background which is at least partially due to manipulation by the hand of man through the use of genetic engineering. For example, the term “transgenic cell”, as used herein, refers to a cell whose DNA contains an exogenous nucleic acid not originally present in the non-transgenic cell. A transgenic cell may be derived or regenerated from a transformed cell or derived from a transgenic cell. Exemplary transgenic cells in the context of the present invention include plant calli derived from a stably transformed plant cell and particular cells (such as leaf, root, stem, or reproductive cells) obtained from a transgenic plant. A “transgenic plant” is any plant in which one or more of the cells of the plant contain heterologous nucleic acid sequences introduced by way of human intervention. Transgenic plants typically express DNA sequences, which confer the plants with characters different from that of native, non-transgenic plants of the same strain. The progeny from such a plant or from crosses involving such a plant in the form of plants, seeds, tissue cultures and isolated tissue and cells, which carry at least part of the modification originally introduced by genetic engineering, are comprised by the definition.

As used herein, the term “genetic probe” refers to a nucleic acid molecule of known sequence, which has its origin in a defined region of the genome and can be a short DNA sequence (or oligonucleotide), a PCR product, or mRNA isolate. Genetic probes are gene-specific DNA sequences to which nucleic acids from a sample (e.g., RNA from a plant extract) are hybridized. Genetic probes specifically bind (or specifically hybridize) to nucleic acid of complementary or substantially complementary sequence through one or more types of chemical bonds, usually through hydrogen bond formation.

As used herein, the term “gene regulatory element” means an element, typically within a nucleic acid, that has the ability to regulate genes, whether it is a by promoting, enhancing, or attenuating expression. In some embodiments, the gene regulatory element is a promoter. In some embodiments, the gene regulatory element is an enhancer. In some embodiments, gene regulatory elements are located at or near the 5′ end of the first exon of a gene. In some embodiment, gene regulatory elements are located within the region of a gene involved in transcriptional and translational initiation.

As used herein the term “heterologous”, when used in reference to genes, refers to genes that are not normally associated with other genetic elements with which they are nevertheless associated (e.g., in a nucleic acid construct) in such an arrangement in nature and/or refers to genes that are associated with such other elements by the hand of man. “Heterologous gene products” refers to products of heterologous genes.

As used herein, the term “lignocellulolytic enzyme polypeptide” refers to a polypeptide that disrupts or degrades lignocellulose, which comprises cellulose, hemicellulose, and lignin. The term “lignocelluloytic enzyme polypeptide” encompasses, but is not limited to cellobiohydrolases, endoglucanases, β-D-glucosidases, xylanases, arabinofuranosidases, acetyl xylan esterases, glucuronidases, mannanases, galactanases, arabinases, lignin peroxidases, manganese-dependent peroxidases, hybrid peroxidases, laccases, ferulic acid esterases and related polypeptides. In some embodiments, disruption or degradation of lignocellulose by a lignocellulolytic enzyme polypeptide leads to the formation of substances including monosaccharides, disaccharides, polysaccharides, and phenols. In some embodiments, a lignocellulolytic enzyme polypeptide shares at least 50%, 60%, 70%, 80% or more overall sequence identity with a polypeptide whose amino acid sequence is set forth in Table 1. Alternatively or additionally, in some embodiments, a lignocellulolytic enzyme polypeptide shows at least 90%, 95%, 96%, 97%, 98%, 99%, or greater identity with at least one sequence element found in a polypeptide whose amino acid sequence is set forth in Table 1, which sequence element is at least 4, 5, 6, 7, 8, 9, 10, 11, 12, 13, 14, 15, 16, 17, 18, 19, 20 or more amino acids long. It will be appreciated that the present invention describes use of lignocellulolytic enzyme polypeptides generally, but also of particular lignocellulolytic enzyme polypeptides (e.g., Acidothermus cellulolyticus E1 endo-1,4-β-glucanase polypeptide, Acidothermus cellulolyticus xylE polypeptide, Acidothermus cellulolyticus gux1 polypeptide, Acidothermus cellulolyticus aviIII polypeptide, and Talaromyces emersonii cbhE polypeptide).

As used herein, the term “mixed linkage glucans” refer to non-cellulosic glucans present in plants and often enriched in seed bran. β-D-glucan residues of mixed-linkage glucans are unbranched but contain both (1→3) and (1→4)-linkages. In some embodiments, enzymes that modify mixed-linkage glucans include laminarinase (E.C. 3.2.1.39), licheninase (E.C. 3.2.1.73/74). In some embodiments, some cellulases can hydrolyze certain (1→4)-linkages.

As used herein, the term “nucleic acid construct” refers to a polynucleotide or oligonucleotide comprising nucleic acid sequences not normally associated in nature. A nucleic acid construct of the present invention is prepared, isolated, or manipulated by the hand of man. The terms “nucleic acid”, “polynucleotide” and “oligonucleotide” are used herein interchangeably and refer to a deoxyribonucleotide (DNA) or ribonucleotide (RNA) polymer either in single- or double-stranded form. For the purposes of the present invention, these terms are not to be construed as limited with respect to the length of the polymer and should also be understood to encompass analogs of DNA or RNA polymers made from analogs of natural nucleotides and/or from nucleotides that are modified in the base, sugar and/or phosphate moieties.

As used herein, the term “operably linked” refers to a relationship between two nucleic acid sequences wherein the expression of one of the nucleic acid sequences is controlled by, regulated by or modulated by the other nucleic acid sequence. In some embodiments, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species.

As will be clear from the context, the term “plant”, as used herein, can refer to a whole plant, plant parts (e.g., cuttings, tubers, pollen), plant organs (e.g., leaves, stems, flowers, roots, fruits, branches, etc.), individual plant cells, groups of plant cells (e.g., cultured plant cells), protoplasts, plant extracts, seeds, and progeny thereof. The class of plants that may be used in the methods of the present invention is as broad as the class of higher plants amenable to transformation techniques, including both monocotyledonous and dicotyledonous plants, as well as certain lower plants such as algae. The term includes plants of a variety of a ploidy levels, including polyploid, diploid and haploid. In certain embodiments of the invention, plants are green field plants. In other embodiments, plants are grown specifically for “biomass energy”. For example, suitable plants include, but are not limited to, alfalfa, bamboo, barley, canola, corn, cotton, cottonwood (e.g. Populus deltoides), eucalyptus, miscanthus, poplar, pine (pinus sp.), potato, rape, rice, soy, sorghum, sugar beet, sugarcane, sunflower, sweetgum, switchgrass, tobacco, turf grass, wheat, and willow. Using transformation methods, genetically modified plants, plant cells, plant tissue, seeds, and the like can be obtained.

As used herein, “plant biomass” refers to biomass that includes a plurality of components found in plant, such as lignin, cellulose, hemicellulose, beta-glucans, homogalacturonans, and rhamnogalacturonans. Plant biomass may be obtained, for example, from a transgenic plant expressing at least one cell wall-modifying enzyme polypeptide as described herein. Plant biomass may be obtained from any part of a plant, including, but not limited to, leaves, stems, seeds, and combinations thereof.

As used herein, the term “polypeptide” generally has its art-recognized meaning of a polymer of at least three amino acids. However, the term is also used to refer to specific functional classes of polypeptides, such as, for example, lignocellulolytic enzyme polypeptides (including, for example, Acidothermus cellulolyticus E1 endo-1,4-β-glucanase polypeptide, Acidothermus cellulolyticus xylE polypeptide, Acidothermus cellulolyticus gux1 polypeptide, Acidothermus cellulolyticus aviIII polypeptide, and Talaromyces emersonii cbhE polypeptide). For each such class, the present specification provides specific examples of known sequences of such polypeptides. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented herein.

As used herein, the term “pretreatment” refers to a thermo-chemical process to remove lignin and hemicellulose bound to cellulose in plant biomass, thereby increasing accessibility of the cellulose to cellulases for hydrolysis. Common methods of pretreatment involve using dilute acid (such as, for example, sulfuric acid), ammonia fiber expansion (AFEX), steam explosion, lime, and combinations thereof.

As used herein, the terms “promoter” and “promoter element” refer to a polynucleotide that regulates expression of a selected polynucleotide sequence operably linked to the promoter, and which effects expression of the selected polynucleotide sequence in cells. The term “plant promoter”, as used herein, refers to a promoter that functions in a plant. In some embodiments of the invention, the promoter is a constitutive promoter, i.e., an unregulated promoter that allows continual expression of a gene associated with it. A constitutive promoter may in some embodiments allow expression of an associated gene throughout the life of the plant. Examples of constitutive plant promoters include, but are not limited to, rice act1 promoter, Cauliflower mosaic virus (CaMV) 35S promoter, and nopaline synthase promoter from Agrobacterium tumefaciens. In some embodiments, the promoter is a promoter from poplar. In some embodiments, the promoter comprises a polynucleotide having a sequence of at least one of SEQ ID NO: 1 to 158. In some embodiments of the invention, the promoter is a tissue-specific promoter that selectively functions in a part of a plant body, such as a flower. In some embodiments of the invention, the promoter is a developmentally specific promoter. In some embodiments of the invention, the promoter is an inducible promoter. In some embodiments of the invention, the promoter is a senescence promoter, i.e., a promoter that allows transcription to be initiated upon a certain event relating to the age of the organism.

As used herein, the term “protoplast” refers to an isolated plant cell without cell walls which has the potency for regeneration into cell culture or a whole plant.

As used herein, the term “regeneration” refers to the process of growing a plant from a plant cell (e.g., plant protoplast, plant callus or plant explant).

As used herein, the term “stably transformed”, when applied to a plant cell, callus or protoplast refers to a cell, callus or protoplast in which an inserted exogenous nucleic acid molecule is capable of replication either as an autonomously replicating plasmid or as part of the host chromosome. The stability is demonstrated by the ability of the transformed cells to establish cell lines or clones comprised of a population of daughter cells containing the exogenous nucleic acid molecule.

As used herein, the term “tempering” refers to a process to condition lignocellulosic biomass prior to pretreatment so as to favor improved yield from hydrolysis and/or allow use of less severe pretreatment conditions without sacrificing yield. In some embodiments, the lignocellulosic biomass transgenically expresses a lignocellulolytic enzyme polypeptide and tempering facilitates activation of the lignocellulolytic enzyme polypeptide. In some embodiments, tempering facilitates improved yield from subsequent hydrolysis as compared to yield obtained from processing without tempering. In some embodiments, tempering facilitates comparable or improved yield from subsequent hydrolysis using less severe pretreatment conditions than would be required without tempering. In some embodiments, tempering comprises a process selected from the group consisting of ensilement, grinding, pelleting, forming a warm water suspension and/or slurry, incubating at a specific temperature, incubating at a specific pH, and combinations thereof. In some embodiments, tempering comprises separating liquid from a slurry that contains soluble sugars and crude enzyme extracts and re-addition of the separated liquid back to the solid biomass after pretreatment. Specific conditions for tempering may depend on specific traits (such as, e.g., traits of the transgene) of the biomass.

As used herein, the term “tissue-preferred”, when used in reference to a gene regulatory element (such as a promoter) or an expression pattern, means characterized by expression preferences in certain tissues. For example, a tissue-preferred promoter can drive and/or facilitate expression that is high in certain tissues (eg. stem) but in low in others.

As used herein, the term “tissue-specific”, when used in reference to a gene regulatory element (such as a promoter) or an expression pattern, means characterized by expression only in certain tissues. For example, a tissue-specific promoter can drive and/or facilitate expression in some tissues but not others.

As used herein, the term “transformation” refers to a process by which an exogenous nucleic acid molecule (e.g., a vector or recombinant DNA molecule) is introduced into a recipient cell, callus or protoplast. The exogenous nucleic acid molecule may or may not be integrated into (i.e., covalently linked to) chromosomal DNA making up the genome of the host cell, callus or protoplast. For example, the exogenous polynucleotide may be maintained on an episomal element, such as a plasmid. Alternatively, the exogenous polynucleotide may become integrated into a chromosome so that it is inherited by daughter cells through chromosome replication. Methods for transformation include, but are not limited to, electroporation, magnetoporation, Ca2+ treatment, injection, particle bombardment, retroviral infection, and lipofection. In some circumstances, an exogenous nucleic acid is introduced in to a cell by mating with another cell. For example, in S. cerevisiae, cells mate with one another.

The term “transgene”, as used herein, refers to an exogenous gene which, when introduced into a host cell through the hand of man, for example, using a process such as transformation, electroporation, particle bombardment, and the like, is expressed by the host cell and integrated into the cell's DNA such that the trait or traits produced by the expression of the transgene is inherited by the progeny of the transformed cell. A transgene may be partly or entirely heterologous (i.e., foreign to the cell into which it is introduced). Alternatively, a transgene may be homologous to an endogenous gene of the cell into which it is introduced, but is designed to be inserted (or is inserted) into the cell's genome in such a way as to alter the genome of the cell (e.g., it is inserted at a location which differs from that of the natural gene or its insertion results in a knockout). A transgene can also be present in a cell in the form of an episome. A transgene can include one or more transcriptional regulatory sequences and other nucleic acids, such as introns. Alternatively or additionally, a transgene is one that is not naturally associated with the vector sequences with which it is associated according to the present invention.

### DETAILED DESCRIPTION OF CERTAIN EMBODIMENTS

In various embodiments, the present invention provides, among other things, novel nucleic acids and vectors comprising novel gene regulatory elements from poplar that can be used to express a gene of interest in a variety of cells, including both monocot and dicot plants. Monocot and dicot transgenic plants expressing heterologous genes under the control of a novel gene regulatory element are also provided. Gene regulatory elements of the present invention include those that, in their endogenous contexts, collectively regulate several classes of genes that are involved in plant cell structure and function, intermediary metabolism, tissue-specific and developmental stage-specific functions. Gene regulatory elements of the present invention collectively demonstrate a useful range of properties with regard to gene expression, including, but not limited to, promoter strength, tissue- and/or developmental stage-specificity, and responsiveness to stimuli.

### I. Nucleic Acids

Nucleic acids of the present invention generally comprise a characteristic sequence corresponding to a novel gene regulatory element from sorghum.

Nucleotide sequences of certain provided sorghum gene regulatory elements are listed as SEQ ID NOs: 1 to 158 and presented in Table 3. In some embodiments, nucleotide sequences of provided nucleic acids comprise a sequence having at least 65%, at least 70%, at least 75%, at least 80%, at least 85%, at least 90%, at least 95%, at least 96%, at least 97%, at least 98%, at least 99%, or more identity to at least one of SEQ ID NO.: 1 to 158. In some embodiments, nucleotide sequences of provided nucleic acids comprise a sequence having at least 65%, at least 70%, at least 75%, at least 80%, at least 85%, at least 90%, at least 95%, at least 96%, at least 97%, at least 98%, at least 99%, or more identity to at least one of SEQ ID NO: 90, 94, 103, 117, 131, 137, 145, and 158. (See, e.g., Examples 2, 3, 4, and 5). In some embodiments, the nucleotide sequences of provided nucleic acids comprise a sequence having at least 65%, at least 70%, at least 75%, at least 80%, at least 85%, at least 90%, at least 95%, at least 96%, at least 97%, at least 98%, at least 99%, or more sequence identity to at least one of SEQ ID NO: 90 and 103.

In many embodiments, provided nucleic acids comprise gene regulatory elements from poplar. In some such embodiments, the gene regulatory elements are promoters, that is, they can drive expression of a gene that is operably linked.

Nucleic acids of the invention may include, in addition to nucleotide sequences described above, sequences that can facilitate manipulations such as molecular cloning. For example, restriction enzyme recognition sites and/or recombinase recognition sites may be included in inventive nucleic acids.

Nucleic acids of the present invention included single stranded and double stranded nucleic acids. DNA, RNA, DNA:RNA heteroduplexes, RNA:RNA duplexes, and DNA-RNA hybrid molecules are contemplated and included. In some embodiments, nucleic acids of the present invention include unconventional nucleotides, chemically modified nucleotides, and/or labeled nucleotides (e.g., radiolabeled, fluorescently labeled, enzymatically labeled, etc.). For example, modifications, labels, and/or use of unconventional nucleotides may facilitate downstream manipulations and/or analyses.

### II. Vectors

Gene vectors of the present invention generally contain a nucleic acid construct that includes one or more expression cassettes for expression of a gene of interest (e.g., a heterologous gene) in a plant of interest. Nucleic acid constructs (also known as “gene constructs”) act as a functional unit that allows expression of a gene of interest. Nucleic acid constructs typically comprise, in addition to the gene of interest (e.g., a heterologous gene whose expression is desired), a gene regulatory element capable of driving expression of the gene of interest (such as a promoter) and a terminator (also known as a stop signal), both of which are operably linked to the gene of interest.

In many embodiments, the gene regulatory element regulates expression of the gene of interest (such as a heterologous gene).

In some embodiments, constructs comprise additional sequences, e.g. marker genes, which are also accompanied by a gene regulatory element (such as a promoter) and a terminator. In many embodiments, the sequences for each of the elements in the construct do not exist in this combination and arrangement in nature and/or are arranged and/or combined by the hand of man.

### A. Expression Cassettes

Expression cassettes generally include 5′ and 3′ regulatory sequences operably linked to a nucleotide sequence encoding a gene of interest.

Techniques used to isolate or clone a gene of interest are known in the art and include isolation from genomic DNA, preparation from cDNA, or a combination thereof. Cloning of a gene from such genomic DNA, can be effected, e.g., by using polymerase chain reaction (PCR) or antibody screening or expression libraries to detect cloned DNA fragments with shared structural features (Innis et al., “PCR: A Guide to Method and Application”, 1990, Academic Press: New York). Alternatively or additionally, other nucleic acid amplification procedures such as ligase chain reaction (LCR), ligated activated transcription (LAT) and nucleotide sequence-based amplification (NASBA) may be used.

Expression cassettes generally include the following elements (presented in the 5′-3′ direction of transcription): a transcriptional and translational initiation region, a coding sequence for a gene of interest, and a transcriptional and translational termination region functional in the organism where it is desired to express the gene of interest (such as a plant).

Other sequences that can be present in a nucleic acid construct include sequences that enhance gene expression (such as, for example, intron sequences and leader sequences). Examples of introns that have been reported to enhance expression include, but are not limited to, introns of the Maize Adh1 gene and introns of the Maize bronze1 gene (J. Callis et. al., Genes Develop. 1987, 1: 1183-1200). Examples of non-translated leader sequences that are known to enhance expression include, but are not limited to, leader sequences from Tobacco Mosaic Virus (TMV, the “omegasequence”), Maize Chlorotic Mottle Virus (MCMV), and Alfalfa Mosaic Virus (AlMV) (see, for example, D. R. Gallie et al., Nucl. Acids Res. 1987, 15: 8693-8711; J. M. Skuzeski et. al., Plant Mol. Biol. 1990, 15: 65-79).

Where appropriate, the gene(s) or polynucleotide sequence(s) encoding the enzyme(s) of interest may be modified to include codons that are optimized for expression in the transformed plant (Campbell and Gowri, Plant Physiol., 1990, 92: 1-11; Murray et al., Nucleic Acids Res., 1989, 17: 477-498; Wada et al., Nucl. Acids Res., 1990, 18: 2367, and U.S. Pat. Nos. 5,096,825; 5,380,831; 5,436,391; 5,625,136, 5,670,356 and 5,874,304). Codon optimized sequences are synthetic sequences, and preferably encode the identical polypeptide (or an enzymatically active fragment of a full length polypeptide which has substantially the same activity as the full length polypeptide) encoded by the non-codon optimized parent polynucleotide.

### 1. Transcriptional and Translational Initiation

Transcriptional initiation regions (also known as gene promoters, which may be said to comprise ‘promoter elements’) in nucleic acid constructs of the present invention can be native or analogous (i.e., found in the native organism such as a plant) and/or foreign or heterologous (i.e., not found in the native plant) to the plant host. Promoters can comprise a naturally occurring sequence and/or a synthetic sequence.

A given nucleic acid construct may contain more than one promoter, for example, in embodiments wherein expression of more than one heterologous gene is desired. In some embodiments, the two or more promoters include promoters that are the same. In the some embodiments, the two or more promoters are different from one another. In some embodiments that involve at least two different promoters, one promoter drives expression of a heterologous gene in cells of one species (such as a species bacterium) while one other promoter drives expression of a heterologous gene in cells of another species (such as a plant species). In some embodiments, the two or more promoters include at least two promoters that drive expression in cells of the same species.

As mentioned previously, the present invention provides in certain embodiments gene regulatory elements from poplar, which include poplar promoters capable of driving gene expression in plants, including poplar and plants other than poplar (including both monocotyledonous and dicotyledonous plants). In many embodiments, provided gene regulatory elements comprise isolated nucleic acids as described above. Nucleotide sequences of certain provided poplar gene regulatory elements are listed as SEQ ID NOs: 1 to 158. In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO.: 1 to 158. In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO:90, 94, 103, 117, 131, 137, 145, and 158. (See, e.g., Examples 2, 3, 4, and 5). In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more sequence identity to at least one of SEQ ID NO: 90 and 103.

Provided gene regulatory elements can be used alone, in combination with each other, and/or in combination with known promoters (such as known plant promoters) to drive and/or facilitate expression of a gene of interest (such as a heterologous gene). For example, in embodiments wherein two heterologous gene products are expressed in the same plant or other organism, expression of one heterologous gene product may be driven and/or facilitated by a gene regulatory element from poplar provided herein, while expression of the other heterologous gene product may be driven and/or facilitated by another second gene regulatory element from poplar provided herein. Alternatively or additionally, expression of one heterologous gene product may be driven and/or facilitated by a gene regulatory element from poplar provided herein, while expression of the other heterologous gene product may be driven and/or facilitated by a known promoter such as a known plant promoter. Any number of heterologous gene products may be expressed with the aid of and/or under the control of any combinations of gene regulatory elements or promoters.

Provided gene regulatory elements include several types of plant promoters, such as constitutive plant promoters, tissue-specific promoters, and developmental-stage specific plant promoters.

In certain embodiments, at least one promoter in the nucleic acid construct is a constitutive plant promoter, i.e., an unregulated promoter that allows continual expression of a gene associated with it. Examples of known plant promoters that can be used in addition to provided gene regulatory elements include, but are not limited to, the 35S cauliflower mosaic virus (CaMV) promoter, a promoter of nopaline synthase, and a promoter of octopine synthase. Examples of other constitutive promoters used in plants are the 19S promoter and promoters from genes encoding actin and ubiquitin. Promoters may be obtained from genomic DNA by using polymerase chain reaction (PCR), and then cloned into the construct.

Constitutive promoters may allow expression of an associated gene throughout the life of an organism such as a plant. In some embodiments, the heterologous gene product is produced throughout the life of the organism. In some embodiments, the heterologous gene product is active throughout the life of the organism. Alternatively or additionally, a constitutive promoter may allow expression of an associated gene in all or a majority of tissues in the organism. In some embodiments, the heterologous gene product is present in all tissues during the life of the organism.

In certain embodiments, at least one promoter in the nucleic acid construct is a tissue-specific plant promoter, i.e., a promoter that allows expression of a gene in a specific tissue or tissues associated with it.

In certain embodiments, at least one promoter in the nucleic acid construct is a tissue-preferred plant promoter, i.e., a promoter that allows preferential expression in one or some tissues (e.g., higher in one or some tissues than in others). For example, a tissue-preferred plant promoter may allow a high level of expression in stem but a low level of expression in leaves and seed.

### 2. Genes of Interest/Heterologous Genes

The gene of interest can be any gene whose expression is desired. In a nucleic acid construct (particularly expression constructs), genes of interest are generally heterologous, i.e., they are not normally associated with the other elements in the construct in such an arrangement in nature and/or they are associated with such other elements by the hand of man. In some embodiments, heterologous gene products (which may be polypeptides and/or RNA molecules) are expressed in cells, tissues, and/or organisms in which they are not expressed in nature; and/or are expressed at levels different than they are expressed in nature.

A given nucleic acid construct may have one or more than one heterologous gene.

a. Enzyme Polypeptides

In some embodiments, the heterologous gene encodes an enzyme polypeptide. A wide variety of enzyme polypeptides may be expressed under the control of, or facilitated by, poplar gene regulatory elements provided by the present invention. A discussion of some classes of such enzyme polypeptides is presented below. The discussion below is not intended to be exhaustive; provided gene regulatory elements may be used to drive and/or facilitate expression of other enzyme polypeptides as well.

i. Lignocellulolytic Enzyme Polypeptides

In some embodiments, the heterologous gene is a lignocellulolytic enzyme polypeptide.

Plants generally comprise lignocellulosic biomass, a complex substrate in which crystalline cellulose is embedded within a matrix of hemicellulose and lignin. Lignocellulose represents approximately 90% of the dry weight of most plant material with cellulose making up between 30% to 50% of the dry weight of lignocellulose and hemicellulose making up between 20% and 50% of the dry weight of lignocellulose.

Disruption and degradation (e.g., hydrolysis) of lignocellulose by lignocellulolytic enzyme polypeptides leads to the formation of substances including monosaccharides, disaccharides, polysaccharides and phenols. In some embodiments, the lignocellulolytic enzyme polypeptide are characterized by and/or are employed under conditions and/or according to a protocol that achieves enhanced disruption and/or degradation of lignocellulose.

Lignocellulolytic enzyme polypeptides whose expression may be driven with gene regulatory elements of the invention include enzymes that are involved in the disruption and/or degradation of lignocellulose. Lignocellulolytic enzyme polypeptides include, but are not limited to, cellulases, hemicellulases and ligninases. Representative examples of lignocellulolytic enzyme polypeptides are presented in Table 1.

TABLE 1
Examples of lignocellulolytic enzyme polypeptides

| | | | GenBank |
|---|---|---|---|
| | | | Accession |
| | | | Number/ |
| Gene | Microbial | Amino Acid Sequence of Exemplary | (SEQ ID |
| name | species | Lignocellulolytic Enzyme Polypeptide | NO.) |
| E1 | Acidothermus | AGGGYWHTSGREILDANNVPVRIAGINWFGFETCNYVVHGLWSRDYRS | AAA75477 |
| | cellulolyticus | MLDQIKSLGYNTIRLPYSDDILKPGTMPNSINFYQMNQDLQGLTSLQV | (SEQ ID NO: |
| | | MDKIVAYAGQIGLRIILDRHRPDCSGQSALWYTSSVSEATWISDLQAL | 177) |
| | | AQRYKGNPTVVGFDLHNEPHDPACWGCGDPSIDWRLAAERAGNAVLSV | |
| | | NPNLLIFVEGVQSYNGDSYWWGGNLQGAGQYPVVLNVPNRLVYSAHDY | |
| | | ATSVYPQTWFSDPTFPNNMPGIWNKNWGYLFNQNIAPVWLGEFGTTLQ | |
| | | STTDQTWLKTLVQYLRPTAQYGADSFQWTFWSWNPDSGDTGGILKDDW | |
| | | QTVDTVKDGYLAPIKSSIFDPVG | |
| gux1 | Acidothermus | MGAPGLRRRLRAGIVSAAALGSLVSGLVAVAPVAHAAVTLKAQYKNND | ABK52390.1 |
| | cellulolyticus | SAPSDNQIKPGLQLVNTGSSSVDLSTVTVRYWFTRDGGSSTLVYNCDW | (SEQ ID NO: |
| | | AAMGCGNIRASFGSVNPATPTADTYLQLSFTGGTLAAGGSTGEIQNRV | 178) |
| | | NKSDWSNFDETNDYSYGTNTTFQDWTKVTVYVNGVLVWGTEPSGATAS | |
| | | PSASATPSPSSSPTTSPSSSPSPSSSPTPTPSSSSPPPSSNDPYIQRF | |
| | | LTMYNKIHDPANGYFSPQGIPYHSVETLIVEAPDYGHETTSEAYSFWL | |
| | | WLEATYGAVTGNWTPFNNAWTTMETYMIPQHADQPNNASYNPNSPASY | |
| | | APEEPLPSMYPVAIDSSVPVGHDPLAAELQSTYGTPDIYGMHWLADVD | |
| | | NIYGYGDSPGGGCELGPSAKGVSYINTFQRGSQESVWETVTQPTCDNG | |
| | | KYGGAHGYVDLFIQGSTPPQWKYTDAPDADARAVQAAYWAYTWASAQG | |
| | | KASAIAPTIAKAAKLGDYLRYSLFDKYFKQVGNCYPASSCPGATGRQS | |
| | | ETYLIGWYYAWGGSSQGWAWRIGDGAAHFGYQNPLAAWAMSNVTPLIP | |
| | | LSPTAKSDWAASLQRQLEFYQWLQSAEGAIAGGATNSWNGNYGTPPAG | |
| | | DSTFYGMAYDWEPVYHDPPSNNWFGFQAWSMERVAEYYYVTGDPKAKA | |
| | | LLDKWVAWVKPNVTTGASWSIPSNLSWSGQPDTWNPSNPGTNANLHVT | |
| | | ITSSGQDVGVAAALAKTLEYYAAKSGDTASRDLAKGLLDSIWNNDQDS | |
| | | LGVSTPETRTDYSRFTQVYDPTTGDGLYIPSGWTGTMPNGDQIKPGAT | |
| | | FLSIRSWYTKDPQWSKVQAYLNGGPAPTFNYHRFWAESDFAMANADFG | |
| | | MLFPSGSPSPTPSPTPTSSPSPTPSSSPTPSPSPSPTGDTTPPSVPTG | |
| | | LQVTGTTTSSVSLSWTASTDNVGVAHYNVYRNGTLVGQPTATSFTDTG | |
| | | LAAGTSYTYTVAAVDAAGNTSAQSSPVTATTASPSPSPSPSPTPTSSP | |
| | | SPTPSPTPSPTSTSGASCTATYVVNSDWGSGFTTTVTVTNTGTRATSG | |
| | | WTVTWSFAGNQTVTNYWNTALTQSGKSVTAKNLSYNNVIQPGQSTTFG | |
| | | FNGSYSGTNTAPTLSCTASZ | |
| XylE | Acidothermus | MGHHAMRRMVTSASVVGVATLAAATVLITGGIAHAASTLKQGAEANGR | ABK51955.1 |
| | cellulolyticus | YFGVSASVNTLNNSAAANLVATQFDMLTPENEMKWDTVESSRGSFNFG | (SEQ ID NO: |
| | | PGDQIVAFATAHNMRVRGHNLVWHSQLPGWVSSLPLSQVQSAMESHIT | 179) |
| | | AEVTHYKGKIYAWDVVNEPFDDSGNLRTDVFYQAMGAGYIADALRTAH | |
| | | AADPNAKLYLNDYNIEGINAKSDAMYNLIKQLKSQGVPIDGVGFESHF | |
| | | IVGQVPSTLQQNMQRFADLGVDVAITELDDRMPTPPSQQNLNQQATDD | |
| | | ANVVKACLAVARCVGITQWDVSDADSWVPGTFSGQGAATMFDSNLQPK | |
| | | PAFTAVLNALSASASVSPSPSPSPSPSPSPSPSPSPSPSPSPSPSPSP | |
| | | SSSPVSGGVKVQYKNNDSAPGDNQIKPGLQVVNTGSSSVDLSTVTVRY | |
| | | WFTRDGGSSTLVYNCDWAVMGCGNIRASFGSVNPATPTADTYLQLSFT | |
| | | GGTLPAGGSTGEIQSRVNKSDWSNFTETNDYSYGTNTTFQDWSKVTVY | |
| | | VNGRLVWGTEPSGTSPSPTPSPSPTPSPSPSPSPSPSPSPSPSPSPSP | |
| | | SSSPSSGCVASMRVDSSWPGGFTATVTVSNTGGVSTSGWQVGWSWPSG | |
| | | DSLVNAWNAVVSVTGTSVRAVNASYNGVIPAGGSTTFGFQANGTPGTP | |
| | | TFTCTTSADLZ | |
| aviIII | Acidothermus | MAATTQPYTWSNVAIGGGGFVDGIVFNEGAPGILYVRTDIGGMYRWDA | ABK52391.1 |
| | cellulolyticus | ANGRWIPLLDWVGWNNWGYNGVVSIAADPINTNKVWAAVGMYTNSWDP | (SEQ ID NO: |
| | | NDGAILRSSDQGATWQITPLPFKLGGNMPGRGMGERLAVDPNNDNILY | 180) |
| | | FGAPSGKGLWRSTDSGATWSQMTNFPDVGTYIANPTDTTGYQSDIQGV | |
| | | VWVAFDKSSSSLGQASKTIFVGVADPNNPVFWSRDGGATWQAVPGAPT | |
| | | GFIPHKGVFDPVNHVLYIATSNTGGPYDGSSGDVWKFSVTSGTWTRIS | |
| | | PVPSTDTANDYFGYSGLTIDRQHPNTIMVATQISWWPDTIIFRSTDGG | |
| | | ATWTRIWDWTSYPNRSLRYVLDISAEPWLTFGVQPNPPVPSPKLGWMD | |
| | | EAMAIDPFNSDRMLYGTGATLYATNDLTKWDSGGQIHIAPMVKGLEET | |
| | | AVNDLISPPSGAPLISALGDLGGFTHADVTAVPSTIFTSPVFTTGTSV | |
| | | DYAELNPSIIVRAGSFDPSSQPNDRHVAFSTDGGKNWFQGSEPGGVTT | |
| | | GGTVAASADGSRFVWAPGDPGQPVVYAVGFGNSWAASQGVPANAQIRS | |
| | | DRVNPKTFYALSNGTFYRSTDGGVTFQPVAAGLPSSGAVGVMFHAVPG | |
| | | KEGDLWLAASSGLYHSTNGGSSWSAITGVSSAVNVGFGKSAPGSSYPA | |
| | | VFVVGTIGGVTGAYRSDDGGTTWVRINDDQHQYGNWGQAITGDPRIYG | |
| | | RVYIGTNGRGIVYGDIAGAPSGSPSPSVSPSASPSLSPSPSPSSSPSP | |
| | | SPSPSSSPSSSPSPSPSPSPSPSRSPSPSASPSPSSSPSPSSSPSSSP | |
| | | SPTPSSSPVSGGVKVQYKNNDSAPGDNQIKPGLQVVNTGSSSVDLSTV | |
| | | TVRYWFTRDGGSSTLVYNCDWAAIGCGNIRASFGSVNPATPTADTYLQ | |
| | | LSFTGGTLAAGGSTGEIQNRVNKSDWSNFTETNDYSYGTNTVFQDWSK | |
| | | VTVYVNGRLVWGTEPSGTSPSPTPSPSPTPSPSPSPSPGGDVTPPSVP | |
| | | TGVVVTGVSGSSVSLAWNASTDNVGVAHYNVYRNGVLVGQPTVTSFTD | |
| | | TGLAAGTAYTYTVAAVDAAGNTSAPSTPVTATTTSPSPSPSPTPSPTP | |
| | | SPTPSPSPSPSLSPSPSPSPSPSPSPSLSPSPSTSPSPSPSPTPSPSS | |
| | | SGVGCRATYVVNSDWGSGFTATVTVTNTGSRATSGWTVAWSFGGNQTV | |
| | | TNYWNTLLTQSGASVTATNLSYNNVIQPGQSTTFGFNATYAGTNTPPT | |
| | | PTCTTNSD | |
| cbhE | Talaromyces | MDPQQAGTATAENHPPLTWQECTAPGSCTTQNGAVVLDANWRWVHDVN | AAL33602.2 |
| | emersonii | GYTNCYTGNTWDPTYCPDDETCAQNCALDGADYEGTYGVTSSGSSLKL | (SEQ ID NO: |
| | | NFVTGSNVGSRLYLLQDDSTYQIFKLLNREFSFDVDVSNLPCGLNGAL | 181) |
| | | YFVAMDADGGVSKYPNNKAGAKYGTGYCDSQCPRDLKFIDGEANVEGW | |
| | | QPSSNNANTGIGDHGSCCAEMDVWEANSISNAVTPHPCDTPGQTMCSG | |
| | | DDCGGTYSNDRYAGTCDPDGCDFNPYRMGNTSFYGPGKIIDTTKPFTV | |
| | | VTQFLTDDGTDTGTLSEIKRFYIQNSNVIPQPNSDISGVTGNSITTEF | |
| | | CTAQKQAFGDTDDFSQHGGLAKMGAAMQQGMVLVMSLDDYAAQMLWLD | |
| | | SDYPTDADPTTPGIARGTCPTDSGVPSDVESQSPNSYVTYSNIKFGPI | |
| | | NSTFTASGD | |

A—Cellulases

Cellulases are lignocellulolytic enzyme polypeptides involved in cellulose degradation. Cellulase enzyme polypeptides are classified on the basis of their mode of action. There are two basic kinds of cellulases: the endocellulases, which cleave the polymer chains internally; and the exocellulases, which cleave from the reducing and non-reducing ends of molecules generated by the action of endocellulases. Cellulases include cellobiohydrolases, endoglucanases, and β-D-glucosidases. Endoglucanases randomly attack the amorphous regions of cellulose substrate, yielding mainly higher oligomers. Cellulobiohydrolases are exocellulases which hydrolyze crystalline cellulose and release cellobiose (glucose dimer). Both types of enzymes hydrolyze β-1,4-glycosidic bonds. β-D glucosidases or cellulobiase converts oligosaccharides and cellubiose to glucose. Beta-glucan glucohydrolase hydrolyzes oligosaccharides to glucose.

According to the present invention, the heterologous gene may encode a cellulase enzyme polypeptide. Transgenic plants of the invention may be engineered to comprise one or more than one gene encoding a cellulase enzyme polypeptide. For example, plants may be engineered to comprise one or more genes encoding a cellulase of the cellubiohydrolase class, one or more genes encoding a cellulase of the endoglucanase class, and/or one or more genes encoding a cellulase of the 13-D glucosidase class.

Examples of endoglucanase genes that can be used in the present invention include those that can be obtained from Aspergillus aculeatus (U.S. Pat. No. 6,623,949; WO 94/14953), Aspergillus kawachii (U.S. Pat. No. 6,623,949), Aspergillus oryzae (Kitamoto et al., Appl. Microbiol. Biotechnol., 1996, 46: 538-544; U.S. Pat. No. 6,635,465), Aspergillus nidulans (Lockington et al., Fungal Genet. Biol., 2002, 37: 190-196), Cellulomonas fimi (Wong et al., Gene, 1986, 44: 315-324), Bacillus subtilis (MacKay et al., Nucleic Acids Res., 1986, 14: 9159-9170), Cellulomonas pachnodae (Cazemier et al., Appl. Microbiol. Biotechnol., 1999, 52: 232-239), Fusarium equiseti (Goedegebuur et al., Curr. Genet., 2002, 41: 89-98), Fusarium oxysporum (Hagen et al., Gene, 1994, 150: 163-167; Sheppard et al., Gene, 1994, 150: 163-167), Humicola insolens (U.S. Pat. No. 5,912,157; Davies et al., Biochem J., 2000, 348: 201-207), Hypocrea jecorina (Penttila et al., Gene, 1986, 45: 253-263), Humicola grisea (Goedegebuur et al., Curr. Genet., 2002, 41: 89-98), Micromonospora cellulolyticum (Lin et al., J. Ind. Microbiol., 1994, 13: 344-350), Myceliophthora thermophila (U.S. Pat. No. 5,912,157), Rhizopus oryzae (Moriya et al., J. Bacteriol., 2003, 185: 1749-1756), Trichoderma reesei (Saloheimo et al., Mol. Microbiol., 1994, 13: 219-228), and Trichoderma viride (Kwon et al., Biosci. Biotechnol. Biochem., 1999, 63: 1714-1720; Goedegebuur et al., Curr. Genet., 2002, 41: 89-98).

In certain embodiments, the heterologous gene encodes the endo-1,4-β-glucanase E1 gene (GenBank Accession No. U33212, See Table 1). This gene was isolated from the thermophilic bacterium Acidothermus cellulolyticus. Acidothermus cellulolyticus has been characterized with the ability to hydrolyze and degrade plant cellulose. The cellulase complex produced by A. cellulolyticus is known to contain several different thermostable cellulase enzymes with maximal activities at temperatures of 75° C. to 83° C. These cellulases are resistant to inhibition from cellobiose, an end product of the reactions catalyzed by endo- and exo-cellulases.

The E1 endo-1,4-β-glucanase is described in detail in U.S. Pat. No. 5,275,944. This endoglucanase demonstrates a temperature optimum of 83° C. and a specific activity of 40 μmol glucose release from carboxymethylcellulose/min/mg protein. This E1 endoglucanase was further identified as having an isoelectric pH of 6.7 and a molecular weight of 81,000 Daltons by SDS polyacrylamide gel electrophoresis. It is synthesized as a precursor with a signal peptide that directs it to the export pathway in bacteria. The mature enzyme polypeptide is 521 amino acids (aa) in length. The crystal structure of the catalytic domain of about 40 kD (358 aa) has been described (J. Sakon et al., Biochem., 1996, 35: 10648-10660). Its pro/thr/ser-rich linker is 60 aa, and the cellulose binding domain (CBD) is 104 aa. The properties of the cellulose binding domain that confer its function are not well-characterized. Plant expression of the E1 gene has been reported (see for example, M. T. Ziegler et al., Mol. Breeding, 2000, 6: 37-46; Z. Dai et al., Mol. Breeding, 2000, 6: 277-285; Z. Dai et al., Transg. Res., 2000, 9: 43-54; and T. Ziegelhoffer et al., Mol. Breeding, 2001, 8: 147-158).

Examples of cellobiohydrolase genes that can be used in the present invention can be obtained from Acidothermus cellulolyticus, Acremonium cellulolyticus (U.S. Pat. No. 6,127,160), Agaricus bisporus (Chow et al., Appl. Environ. Microbiol., 1994, 60: 2779-2785), Aspergillus aculeatus (Takada et al., J. Ferment. Bioeng., 1998, 85: 1-9), Aspergillus niger (Gielkens et al., Appl. Environ. Microbiol., 65: 1999, 4340-4345), Aspergillus oryzae (Kitamoto et al., Appl. Microbiol. Biotechnol., 1996, 46: 538-544), Athelia rolfsii (EMBL accession No. AB103461), Chaetomium thermophilum (EMBL accession Nos. AX657571 and CQ838150), Cullulomonas fimi (Meinke et al., Mol. Microbiol., 1994, 12: 413-422), Emericella nidulans (Lockington et al., Fungal Genet. Biol., 2002, 37: 190-196), Fusarium oxysporum (Hagen et al., Gene, 1994, 150: 163-167), Geotrichum sp. 128 (EMBL accession No. AB089343), Humicola grisea (de Oliviera and Radford, Nucleic Acids Res., 1990, 18: 668; Takashima et al., J. Biochem., 1998, 124: 717-725), Humicola nigrescens (EMBL accession No. AX657571), Hypocrea koningii (Teeri et al., Gene, 1987, 51: 43-52), Mycelioptera thermophila (EMBL accession No. AX657599), Neocallimastix patriciarum (Denman et al., Appl. Environ. Microbiol., 1996, 62: 1889-1896), Phanerochaete chrysosporium (Tempelaars et al., Appl. Environ. Microbiol., 1994, 60: 4387-4393), Thermobifida fusca (Zhang, Biochemistry, 1995, 34: 3386-3395), Trichoderma reesei (Terri et al., BioTechnology, 1983, 1: 696-699; Chen et al., BioTechnology, 1987, 5: 274-278), and Trichoderma viride (EMBL accession Nos. A4368686 and A4368688).

Examples of β-D-glucosidase genes that can be used in the present invention can be obtained from Aspergillus aculeatus (Kawaguchi et al., Gene, 1996, 173: 287-288), Aspergillus kawachi (Iwashita et al., Appl. Environ. Microbiol., 1999, 65: 5546-5553), Aspergillus oryzae (WO 2002/095014), Cellulomonas biazotea (Wong et al., Gene, 1998, 207: 79-86), Penicillium funiculosum (WO 200478919), Saccharomycopsis fibuligera (Machida et al., Appl. Environ. Microbiol., 1988, 54: 3147-3155), Schizosaccharomyces pombe (Wood et al., Nature, 2002, 415: 871-880), and Trichoderma reesei (Barnett et al., BioTechnology, 1991, 9: 562-567).

Other examples of cellulases that can be used in accordance with the present invention include family 48 glycoside hydrolases such as gux1 from Acidothermus cellulolyticus, avicelases such as aviIII from Acidothermus cellulolyticus, and cbhE from Talaromyces emersonii. (See Table 1.)

Transgene expression of cellulases in plants for the conversion of cellulose to glucose has been reported (see, for example, Y. Jin Cai et al., Appl. Environ. Microbiol., 1999, 65: 553-559; C. R. Sanchez et al., Revista de Microbiologica, 1999, 30: 310-314; R. Cohen et al., Appl. Environ., 2995, 71: 2412-2417; Z. Dai et al., Transg. Res., 2005, 14: 627-543).

B—Hemicellulases

Hemicellulases are lignocellulolytic enzyme polypeptides that are involved in hemicellulose degradation. Hemicellulases include xylanases, arabinofuranosidases, acetyl xylan esterases, ferulic acid esterases, xyloglucanases, β-glucanases, β-xylosidases, glucuronidases, mannanases, galactanases, and arabinases. Similar to cellulase enzyme polypeptides, hemicellulases are classified on the basis of their mode of action: the endo-acting hemicellulases attack internal bonds within the polysaccharide chain; the exo-acting hemicellulases act progressively from either the reducing or non-reducing end of polysaccharide chains.

According to the present invention, heterologous genes may encode a hemicellulase enzyme polypeptide. Transgenic plants of the invention may be engineered to comprise one or more than one gene encoding a hemicellulase enzyme polypeptide. For example, plants may be engineered to comprise one or more genes encoding a hemicellulase of the xylanase class, one or more genes encoding a hemicellulase of the arabinofuranosidase class, one or more genes encoding a hemicellulase of the acetyl xylan esterase class, one or more genes encoding a hemicellulase of the glucuronidase class, one or more genes encoding a hemicellulase of the mannanase class, one or more genes encoding a hemicellulase of the galactanase class, and/or one or more genes encoding a hemicellulase of the arabinase class.

Examples of endo-acting hemicellulases include endoarabinanase, endoarabinogalactanase, endoglucanase, endomannanase, endoxylanase, and feraxan endoxylanase. Examples of exo-acting hemicellulases include α-L-arabinosidase, β-L-arabinosidase, α-1,2-L-fucosidase, α-D-galactosidase, β-D-galactosidase, β-D-glucosidase, β-D-glucuronidase, β-D-mannosidase, β-D-xylosidase, exo-glucosidase, exo-mannobiohydrolase, exo-mannanase, exo-xylanase, xylan α-glucuronidase, and coniferin β-glucosidase.

Hemicellulase genes can be obtained from any suitable source, including fungal and bacterial organisms, such as Aspergillus, Disporotrichum, Penicillium, Neurospora, Fusarium, Trichoderma, Humicola, Thermomyces, and Bacillus. Examples of hemicellulases that can be used in the present invention can be obtained from Acidothermus cellulolyticus, Acidobacterium capsulatum (Inagaki et al., Biosci. Biotechnol. Biochem., 1998, 62: 1061-1067), Agaricus bisporus (De Groot et al., J. Mol. Biol., 1998, 277: 273-284), Aspergillus aculeatus (U.S. Pat. No. 6,197,564; U.S. Pat. No. 5,693,518), Aspergillus kawachii (Ito et al., Biosci. Biotechnol. Biochem., 1992, 56: 906-912), Aspergillus niger (EMBL accession No. AF108944), Magnaporthe grisea (Wu et al., Mol. Plant. Microbe Interact., 1995, 8: 506-514), Penicillium chrysogenum (Haas et al., Gene, 1993, 126: 237-242), Talaromyces emersonii (WO 02/24926), and Trichoderma reesei (EMBL accession Nos. X69573, X69574, and AY281369).

In certain embodiments, the heterologous gene comprises the A. cellulolyticus endoxylanase xylE.

C—Ligninases

Ligninases are lignocellulolytic enzyme polypeptides that are involved in the degradation of lignin. Lignin-degrading enzyme polypeptides include, but are not limited to, lignin peroxidases, manganese-dependent peroxidases, hybrid peroxidases (which exhibit combined properties of lignin peroxidases and manganese-dependent peroxidases), and laccases. Hydrogen peroxide, required as co-substrate by the peroxidases, can be generated by glucose oxidase, aryl alcohol oxidase, and/or lignin peroxidase-activated glyoxal oxidase.

According to the present invention, heterologous genes may encode a ligninase enzyme polypeptide. Transgenic plants of the invention may be engineered to comprise one or more than one gene encoding a ligninase enzyme polypeptide. For example, plants may be engineered to comprise one or more genes encoding a ligninase of the lignin peroxidase class, one or more genes encoding a ligninase of the manganese-dependent peroxidase class, one or more genes encoding a ligninase of the hybrid peroxidase class, and/or one or more genes encoding a ligninase of the laccase class.

Lignin-degrading genes may be obtained from Acidothermus cellulolyticus, Bjerkandera adusta, Ceriporiopsis subvermispora (see WO 02/079400), Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride.

Examples of genes encoding ligninases that can be used in the invention can be obtained from Bjerkandera adusta (WO 2001/098469), Ceriporiopsis subvermispora (Conesa et al., J. Biotechnol., 2002, 93: 143-158), Cantharellus cibariusi (Ng et al., Biochem. and Biophys. Res. Comm., 2004, 313: 37-41), Coprinus cinereus (WO 97/008325; Conesa et al., J. Biotechnol., 2002, 93: 143-158), Lentinula edodes (Nagai et al., Applied Microbiol. and Biotechnol., 2002, 60: 327-335, 2002), Melanocarpus albomyces (Kiiskinen et al., FEBS Letters, 2004, 576: 251-255, 2004), Myceliophthora thermophila (WO 95/006815), Phanerochaete chrysosporium (Conesa et al., J. Biotechnol., 2002, 93: 143-158; Martinez, Enz, Microb, Technol, 2002, 30: 425-444), Phlebia radiata (Conesa et al., J. Biotechnol., 2002, 93: 143-158), Pleurotus eryngii (Conesa et al., J. Biotechnol., 2002, 93: 143-158), Polyporus pinsitus (WO 96/000290), Rigidoporus lignosus (Garavaglia et al., J. of Mol. Biol., 2004, 342: 1519-1531), Rhizoctonia solani (WO 96/007988), Scytalidium thermophilum (WO 95/033837), Tricholoma giganteum (Wang et al., Biochem. Biophys. Res. Comm., 2004, 315: 450-454), and Trametes versicolor (Conesa et al., J. Biotechnol., 2002, 93: 143-158).

For example, transgenic plants of the invention may be engineered to comprise one or more lignin peroxidases. Genes encoding lignin peroxidases may be obtained from Phanerochaete chrysosporium or Phlebia radiata. Lignin-peroxidases are glycosylated heme proteins (MW 38 to 46 kDa) which are dependent on hydrogen peroxide for activity and catalyze the oxidative cleavage of lignin polymer. At least six (6) heme proteins (H1, H2, H6, H7, H8 and H10) with lignin peroxidase activity have been identified Phanerochaete chrysosporium in strain BKMF-1767. In certain embodiments, plants are engineered to comprise the white rot filamentous Phanerochaete chrysosporium ligninase (CGLS) (H. A. de Boer et al., Gene, 1988, 69(2): 369) (see the Examples section).

D—Other Lignocellulolytic Enzyme Polypeptides

In addition to cellulases, hemicellulases and ligninases, lignocellulolytic enzyme polypeptides that can be used in the practice of the present invention also include enzymes that degrade pectic substances or phenolic acids such as ferulic acid. Pectic substances are composed of homogalacturonan (or pectin), rhamno-galacturonan, and xylogalacturonan. Enzymes that degrade homogalacturonan include pectate lyase, pectin lyase, polygalacturonase, pectin acetyl esterase, and pectin methyl esterase. Enzymes that degrade rhamnogalacturonan include alpha-arabinofuranosidase, beta-galactosidase, galactanase, arabinanase, alpha-arabinofuranosidase, rhamnogalacturonase, rhamnogalacturonan lyase, and rhamnogalacturonan acetyl esterase. Enzymes that degrade xylogalacturonan include xylogalacturonosidase, xylogalacturonase, and rhamnogalacturonan lyase.

Phenolic acids include ferulic acid, which functions in the plant cell wall to cross-link cell wall components together. For example, ferulic acid may cross-link lignin to hemicellulose, cellulose to lignin, and/or hemicellulose polymers to each other. Ferulic acid esterases cleave ferulic acid, disrupting the cross linkages.

Other enzymes that may enhance or promote lignocellulose disruption and/or degradation may be expressed under the control of a gene regulatory element provided in the present disclosure and include, but are not limited to, amylases (e.g., alpha amylase and glucoamylase), esterases, lipases, phospholipases, phytases, proteases, and peroxidases.

E—Combinations of Lignocellulolytic Enzyme Polypeptides

According to the present invention, heterologous genes may encode a lignocellulolytic enzyme polypeptide, e.g., a cellulase enzyme polypeptide, a hemicellulase enzyme polypeptide, or a ligninase enzyme polypeptide. Transgenic plants of the invention may be engineered to comprise one or more than one gene encoding lignocellulolytic enzyme polypeptides, e.g., enzymes from different classes of cellulases, enzymes from different classes of hemicellulases, enzymes from different classes of ligninases, or any combinations thereof. For example, combinations of genes may be selected to provide efficient degradation of one component of lignocellulose (e.g., cellulose, hemicellulose, or lignin). Alternatively, combinations of genes may be selected to provide efficient degradation of the lignocellulosic material.

In certain embodiments, genes are optimized for the substrate (e.g., cellulose, hemicellulase, lignin or whole lignocellulosic material) in a particular plant (e.g., corn, tobacco, switchgrass). Tissue from one plant species is likely to be physically and/or chemically different from tissue from another plant species. Selection of genes or combinations of genes to achieve efficient degradation of a given plant tissue is within the skill of artisans in the art.

In some embodiments, combinations of genes are selected to provide for synergistic enzyme activity (i.e., genes are selected such that the interaction between distinguishable enzyme polypeptides or enzyme activities results in the total activity of the enzymes taken together being greater than the sum of the effects of the individual activities).

Efficient lignocellulolytic activity may be achieved by production of two or more enzyme polypeptides in a single transgenic plant. As mentioned above, plants may be transformed to express more than one enzyme polypeptide, for example, by employing the use of multiple gene constructs encoding each of the selected enzymes or a single construct comprising multiple nucleotide sequences encoding each of the selected enzymes. Alternatively, individual transgenic plants, each stably transformed to express a given enzyme, may be crossed by methods known in the art (e.g., pollination, hand detassling, cytoplasmic male sterility, and the like) to obtain a resulting plant that can produce all the enzymes of the individual starting plants.

Alternatively or additionally, efficient lignocellulolytic activity may be achieved by production of two or more lignocellulolytic enzyme polypeptides in separate plants. For example, three separate lines of plants (e.g., corn), one expressing one or more enzymes of the cellulase class, another expressing one or more enzymes of the hemicellulase class and the third one expressing one or more enzymes of the ligninase class, may be developed and grown simultaneously. The desired “blend” of enzymes produced may be achieved by simply changing the seed ratio, taking into account farm climate and soil type, which are expected to influence enzyme yields in plants.

Other advantages of this approach include, but are not limited to, increased plant health (which is known to be adversely affected as the number of introduced genes increases), simpler transformations procedures and great flexibility in incorporating the desired traits in commercial plant varieties for large-scale production.

G—Thermophilic and Thermostable Enzyme Polypeptides

It may be sometimes desirable to expressing thermophilic and/or thermostable enzyme polypeptides. Gene regulatory elements provided by the present invention may be used to drive and/or facilitate expression of genes encoding such polypeptides as well. For example, enzyme polypeptides whose optimal range of temperature for activity (thermophilic enzyme polypeptides) may be expressed in transgenic plants in accordance with the invention. Without wishing to be bound by any particular theory, the limited activity or absence of activity during growth of the plant (at moderate or low temperatures, at which the enzyme polypeptide is less active) may be beneficial to the health of the plant. Alternatively or additionally, and without wishing to be bound by any particular theory, such enzyme polypeptides may facilitate increased hydrolysis because of their high activity at high temperature conditions commonly used in the processing of cellulosic biomass.

In some embodiments, the present invention provides a transgenic plant, the genome of which is augmented with a recombinant polynucleotide encoding at least one lignocellulolytic enzyme polypeptide that exhibits low activity at a temperature below about 60° C., below about 50° C., below about 40° C., or below about 30° C. In some embodiments, the present invention provides a transgenic plant, the genome of which is augmented with a recombinant polynucleotide encoding at least one lignocellulolytic enzyme polypeptide that exhibits high activity at a temperature above about 50° C., above about 60° C., above about 70° C., above about 80° C., or above about 90° C.

In some embodiments, the present invention provides a transgenic plant, the genome of which is augmented with a recombinant polynucleotide encoding at least one lignocellulolytic enzyme polypeptide that is or is homologous to a lignocellulolytic enzyme polypeptide found in a thermophilic microorganism (e.g., bacterium, fungus, etc.). In some such embodiments, the thermophilic organism is a bacterium that is a member of a genus selected from the group consisting of Aeropyrum, Acidilobus, Acidothermus, Aciduliprofundum, Anaerocellum, Archaeoglobus, Aspergillus, Bacillus, Caldibacillus, Caldicellulosiruptor, Caldithrix, Cellulomonas, Chaetomium, Chloroflexus, Clostridium, Cyanidium, Deferribacter, Desulfotomaculum, Desulfurella, Desulfurococcus, Fervidobacterium, Geobacillus, Geothermobacterium, Humicola, Ignicoccus, Marinitoga, Methanocaldococcus, Methanococcus, Methanopyrus, Methanosarcina, Methanothermobacter, Nautilia, Pyrobaculum, Pyrococcus, Pyrodictium, Rhizomucor, Rhodothermus, Staphylothermus, Scylatidium, Spirochaeta, Sulfolobus, Talaromyces, Thermoascus, Thermobifida, Thermococcus, Thermodesulfobacterium, Thermodesulfovibrio, Thermomicrobium, Thermoplasma, Thermoproteus, Thermothrix, Thermotoga, Thermus, and Thiobacillus; in some such embodiments, the thermophilic microorganism is a bacterium that is a member of a species selected from the group consisting of Acidothermus cellulolyticus, Pyrococcus furiosus, and Talaromyces emersonii.

ii. Cell Wall-Modifying Enzyme Polypeptides

In some embodiments, the heterologous gene (whose expression is driven by a provided gene regulatory element) encodes a cell wall-modifying enzyme polypeptide described in U.S. patent application Ser. No. 12/476,247 (filed on Jun. 1, 2009), the contents of which are herein incorporated by reference in their entirety. In some embodiments, cell wall-modifying enzyme polypeptides are lignocelluloytic enzyme polypeptides.

Cell wall-modifying enzyme polypeptides useful in accordance with the present invention include those having at least 50%, 60%, 70%, 80% or more overall sequence identity with a polypeptide whose amino acid sequence is set forth in Table 1 of U.S. patent application Ser. No. 12/476,247. Alternatively or additionally, in some embodiments, cell wall-modifying enzyme polypeptide shows at least 90%, 95%, 96%, 97%, 98%, 99%, or greater identity with at least one sequence element found in a polypeptide whose amino acid sequence is set forth in Table 1 of U.S. patent application Ser. No. 12/476,247, which sequence element is at least 4, 5, 6, 7, 8, 9, 10, 11, 12, 13, 14, 15, 16, 17, 18, 19, 20 or more amino acids long.

A variety of organisms produce cell wall-modifying enzyme polypeptides. Cell wall-modifying enzyme polypeptides may have, for example, archaeal, fungal, insect, animal, or plant origins.

In some embodiments, the cell wall-modifying enzyme polypeptide has cellulase activity. In some embodmients, the cell wall-modifying enzyme polypeptide has an activity selected from the group consisting of feruloyl esterase (also known as ferulic acid esterase), xylanase, alpha-L-arabinofuranosidase, endogalactanase, acetylxylan esterase, beta-xylosidase, xyloglucanase, glucuronoyl esterase, endo-1,5-alpha-L-arabinosidase, pectin methylesterase, endopolygalacturonase, exopolygalacturonase, pectin lyase, pectate lyase, rhamnogalacturonan lyase, pectin acetylesterase, alpha-L-rhamnosidase, mannanase, exoglucanase, glucan glycohydrolase, licheninase, laminarinase, beta-(1,3)-(1,4)-glucanase and beta-glucosidase activity. Such activities may be similar to that of other enzyme polypeptides, including those known in the art that are classified by an EC class and/or listed in enzyme databases (such as CaZY, www.cazy.org, which lists carbohydrate-active enzymes).

In some embodiments, the cell wall-modifying enzyme polypeptide modifies a plant cell wall component. In many such embodiments, the cell wall-modifying enzyme polypeptide modifies the plant cell wall component in such a way that the plant biomass is more amenable to processing steps (e.g., enzymatic digestion). For example, cell wall-modifying enzyme polypeptides may modify plant cell wall components in such a way as to allow increased digestability, increased hydrolysis, and/or increased sugar yields.

In some embodiments, modifying comprises cleavage and/or hydrolysis of the plant cell wall component. Examples of plant cell wall components that may be modified include, but are not limited to, xylans, xylan side chains, glucuronoarabinoxylans, xyloglucans, mixed-linkage glucans, pectins, pectates, rhamnogalacturonans, rhamnogalacturonan side chains, lignin, cellulose, mannans, galactans, arabinans, oligosaccharides derived from cell wall polysaccharides, and combinations thereof.

In some embodiments, the cell wall-modifying enzyme polypeptide disrupts an interaction in the plant biomass such as a covalent linkage, an ionic bonding interaction, a hydrogen bonding interaction, or a combination thereof. Examples of linkages that may be disrupted include, but are not limited to, hemicellulose-cellulose-lignin, hemicellulose-cellulose-pectin, hemicellulose-diferululate-hemicellulose, hemicellulose-ferulate-lignin, mixed beta-D-glucan-cellulose, mixed-beta-D-glucan-hemicellulose, pectin-ferulate-lignin linkages, and combinations thereof. In some embodiments, disrupting comprises hydrolyzing a linkage, such as a feruloyl ester linkage.

b. Heterologous Gene Products Conferring Resistance to Pests, Disease, and Environmental Stress

Heterologous genes may express products that confer benefits) to the transgenic plant such as herbicide resistance, insect resistance, disease resistance, resistance against parasites, and/or increased tolerance to environmental stress (e.g., drought).

Herbicide Resistance

A number of gene products are known in the art that can confer resistance to herbicides. For example, glyphosate (N-(phosphonomethyl) glycine) is a broad-spectrum systemic herbicide and the active ingredient of ROUNDUP™ formulations. Glyphosate acts by inhibiting 5-enolpyruvoyl-shikimate-3-phosphate synthetase (EPSPS) (encoded in some organisms by the aroA gene), starving the affected cells for aromatic amino acids. Some micro-organisms have a mutant form of EPSPS that is resistant to glyphosate inhibition, and this form of the enzyme can be used to impart glyphosate resistance.

As a further example, the herbicide bromoxynil (marketed as Buctril) is applied post-emergence to kill broadleaf weeds, and works by inhibiting photosynthesis in plants. Bromoxynil nitrilase (BXN), a gene from the bacterium Klebsiella pneumoniae, detoxifies bromoxynil in genetically engineered plants and therefore can confer resistance to herbicides.

The L-isomer of phosphinothricin (PPT, glufosinate ammonium) is the active ingredient of several commercial broad spectrum herbicide formulation. An analogue of L-glutamic acid, PPT, is a competitive inhibitor of glutamine synthetase, the only enzyme that can catalyze assimilation of ammonia into glutamic acid into plants. Inhibition of glutamine synthetase ultimately results in the accumulation of toxic ammonia levels, resulting in plant cell death. Phosphosphinothricin acetyltransferase, which is encoded by the bar gene from Streptomyces hygroscopicus, confers resistance to herbicides that contain PPT.

Dalapon is an herbicide used to control grasses in a wide variety of crops. Dalapon dehalogenase is capable of degrading high concentrations of the herbicide dalapon.

Additional non-limiting examples of genes that provide resistance to herbicides include, but are not limited to, mutant genes that confer resistance to imidazalinone or sulfonylurea, such as genes encoding mutant form of acetohydroxyacid synthase (AHAS), also known as acetolactate synthase (ALS) (Lee at al., EMBO J., 1988, 7: 1241; Miki et al., Theor. Appl. Genet., 1990, 80: 449; and U.S. Pat. No. 5,773,702); and genes that confer resistance to phenoxy propionic acids and cyclohexones such as the ACCAse inhibitor-encoding genes (Marshall et al., Theor. Appl. Genet., 1992, 83: 435).

Resistance to Pests and/or Diseases

Genes that confer resistance to pests and/or disease include, but are not limited to, genes whose products confer resistance to infestation from an organism selected from the group consisting of insects, bacteria, fungi, and nematodes. Heterologous genes whose products confer resistance to viruses may also be expressed using gene regulatory elements of the present invention.

Gene products that can confer resistance to insects and/or insect disease include, but are not limited to, Bt (Bacillus thuringiensis) proteins (such as delta-endotoxin (U.S. Pat. No. 6,100,456)); vitamin-binding proteins such as avidin and avidin homologs (which can be used as larvicides against insect pests); insect-specific hormones or pheromones such as ecdysteroid and juvenile hormone, and variants thereof, mimetics based thereon, or an antagonists or agonists thereof; insect-specific peptides or neuropeptides which, upon expression, disrupts the physiology of the pest; insect-specific venom such as that produced by a wasp, snake, etc.; enzyme polypeptides responsible for the accumulation of monoterpenes, sesquiterpenes, asteroid, hydroxamic acid, phenylpropanoid derivative or other non-protein molecule with insecticidal activity; insect-specific antibodies or antitoxins (Tavladoraki et al., Nature, 1993, 366: 469); and TcdA protein (Liu et al., 2003 Nature Biotechnology 21: 1222-1228).

Gene products that can confer resistance to bacteria and/or bacterial diseases include, but are not limited to, nucleotide-binding-sequence LRR (also known as ‘NBS-leucine rich repeat’) proteins (Van Der Biezen and Jones, 1998 Trends in Biochemical Sciences 23: 454-456).

Gene products that can confer resistance to fungi and/or fungal diseases include, but are not limited to, Pi-ta (U.S. Pat. No. 6,743,969), Pathogenesis-related (PR) proteins, chitinases and β-1,3-glucanases, ribosome-inactivating proteins (RIPs), thionins, hydrophobic moment peptides (such as derivatives of Tachyplesin which inhibit fungal pathogens), and antifungal peptides such as LCI.

Gene products that can confer resistance to viruses and/or viral diseases include, but are not limited to, nucleotide-binding site-leucine-rich repeat (NBS-LRR proteins), virus-specific antibodies and antitoxins (Tavladoraki et al., Nature, 1993, 366: 469), viral invasive proteins or complex toxins derived therefrom (Beachy et al., Ann. Rev. Phytopathol., 1990, 28: 451), PR proteins, and Rx proteins (genetically engineered cross protection is conferred by expressing viral coat protein genes in the plant genome).

Gene products that can confer resistance to nematodes and/or nematode diseases include, but are not limited to, peroxidases, chitinases, lipoxygenases, proteinase inhibitors, Mi proteins, Gro, Gpa and Cre proteins.

Other gene products that can confer resistances to diseases or pests include, but are not limited to, lectins (Van Damme et al., Plant Mol. Biol., 1994, 24: 825); protease or amylase inhibitors, such as the rice cysteine proteinase inhibitor (Abe et al., J. Biol. Chem., 1987, 262: 16793) and the tobacco proteinase inhibitor I (Hubb et al., Plant Mol. Biol., 1993, 21: 985); enzyme polypeptides involved in the modification of a biologically active molecule (U.S. Pat. No. 5,539,095); peptides that stimulate signal transduction; membrane permeases (channel formers or channel blockers) (Jaynes et al., Plant Sci., 1993, 89: 43); and developmental-arrestive proteins produced by a plant, pathogen or parasite that prevents disease.

Resistance to Stress

Gene products that confer resistance to environmental stress include both biotic and abiotic stress proteins.

Biotic stress in plants can be caused by bacteria, fungi, viruses, insects and nematodes. Non-limiting examples of proteins that can provide biotic stress resistance/tolerance in plants include those that confer resistance to diseases and pests mentioned above, as well as DREB transcription factors (Agarwal et al., 2006 Plant Cell Reports 25: 1263-1274) and MAP Kinases (U.S. Pat. No. 7,345,219).

Abiotic stress in plants can be caused by a variety of factors, including, but not limited to, nutrient imbalances, light (high light, UV, darkness), water imbalances (deficit, desiccation, flooding), temperature imbalances (frost, cold, heat), oxidation stress, hypoxia, physical factors (such as wind and touch), salt, and heavy metals. Examples of gene products that can provide abiotic stress resistance/tolerance in plants include HSFs, LEAs, CORs, CBFs and ABFs (Vinocur and Altman, 2005 Current Opinion in Biotechnology 16:123-132).

Examples of genes whose products confer resistance to environmental stress include, but are not limited to, mtld and HVA1 (which confer resistance to environmental stress factors); and rd29A and rd19B (Arabidopsis thaliana genes that encode hydrophilic proteins induced in response to dehydration, low temperature, salt stress, and/or exposure to abscisic acid and enable the plant to tolerate the stress (Yamaguchi-Shinozaki et al., Plant Cell, 1994, 6: 251-264)). Other such genes contemplated can be found in U.S. Pat. Nos. 5,296,462 and 5,356,816.

c. Other Heterologous Gene Products

Gene regulatory elements provided by the present invention may also be used to drive and/or facilitate other heterologous gene products that confer advantages to the plants that express them.

For example, nutrient utilization polypeptides can be expressed in transgenic plants. Such polypeptides can maximize utilization of nutrients by plants and may lead to increased yields. Nutrients whose utilization maximization may be desired include, but are not limited to, nitrogen, phosphorous, potassium, iron, zinc etc.

It may be desirable to trnasgenically express anthranilate synthase, which catalyzes the conversion of chorismate into anthranilate. Anthranilate is the biosynthetic precursor of both tryptophan and numerous secondary metabolites, including inducible plant defense compounds.

It may be desirable to express mycotoxin reduction polypeptides in plants. Mycotoxins are toxic and carcinogenic chemicals produced by fungi in plants during growth or storage of grains and are major concern for growers. Bt proteins, when expressed in plants reduce mycotoxin content (Wu et al., 2004 Toxin Reviews 23: 397-424).

Male sterility polypeptides may also be expressed in transgenic plants using gene regulatory elements of the present invention. Male sterility in plants can be induced by expressing several types of polypeptides such as RNase/Barnase (Mariani et al., 1990 Nature 347: 737-741).

Heterologous gene products that affect grain composition or quality (e.g., by altering key components of grain, such as starch, protein, bran, etc.) may also be expressed. Desired changes in composition may include, for example, relative proportions of starch fractions such amylose and amylopectin; decreased amounts of undesirable components such as phytic acid; and/or improved amino acid content conferred, for example, by modified seed storage proteins that have been. For example, corn zeins modified to contain more lysine can be expressed.

Polypeptides having therapeutic value can also be expressed in plants using provided gene regulatory elements. Such polypeptides can be harvested from plants transgenically expressing them and then purified for downstream applications. Such polypeptides include, but are not limited to, antibodies, blood products, cytokines, growth factors, hormones, recombinant enzymes, and vaccines that would have a variety of applications in human and animal health. For example, lactoferrin and lysozyme has been produced in rice grains (Ventria Bioscience).

Heterologous gene products that may be expressed also include RNA molecules, for example, those that regulate a plant gene.

### 3. Transcriptional and Translational Termination

The transcriptional and translational termination region generally comprises a sequence that encodes a “terminator” (the “terminator sequence”). The transcriptional and translational termination region can be native with the transcription initiation region, can be native with the operably linked polynucleotide sequence of interest, and/or can be derived from another source. Convenient termination regions are available from the T1-plasmid of A. tumefaciens, such as the octopine synthase and nopaline synthase termination regions (An et al., Plant Cell, 1989, 1: 115-122; Guerineau et al., Mol. Gen. Genet. 1991, 262: 141-144; Proudfoot, Cell, 1991, 64: 671-674; Sanfacon et al., Genes Dev. 1991, 5: 141-149; Mogen et al., Plant Cell, 1990, 2:1261-1272; Munroe et al., Gene, 1990, 91:151-158; Ballas et al., Nucleic Acids Res., 1989, 17: 7891-7903; and Joshi et al., Nucleic Acid Res., 1987, 15: 9627-9639).

### 4. Marker Genes

In some embodiments, nucleic acid constructs include one or more marker genes. Marker genes are genes that impart a distinct phenotype to cells expressing the marker gene and thus allow transformed cells to be distinguished from cells that do not have the marker. Such genes may encode, for example, a selectable and/or screenable marker. In some embodiments, nucleic acid constructs comprise a marker that allows selecting and/or screening in a transformed cell.

In some embodiments, the transformed cell is grown in culture medium under conditions that select for cells that either have (positive selection) or do not have (negative selection) the marker. In some embodiments, a combination of positive and negative selection is used.

In some so-called positive selection schemes, most cells in a population are unable to divide and because they lack the ability to use a nutrient (such as, for example, a carbon source) present in the selection medium. In these schemes, the selectable marker confers an ability to use the nutrent. Thus, cells that have the selectable marker gain an advantage over other cells in the population and therefore can be selected.

In some so-called negative screening/selection schemes, most cells in a population are unable to divide because of the effects of a toxic agent (such as, for example, an antibiotic present in the selection medium). In these schemes, the selectable marker confers an ability to overcome the toxicity (for example, by blocking uptake or by chemically modifying the toxic agent). Thus, cells that have the selectable marker gain an advantage over other cells in the population and therefore can be selected.

In some embodiments, the transformed cell undergoing selection is a prokaryotic cell, such as E. coli and Agrobacterium. In some embodiments, the transformed cell undergoing selection is a eukaryotic cell, such as a yeast (for example, S. cerevisiae), mammalian, insect, or plant cell.

In some embodiments, the characteristic phenotype allows the identification of cells, groups of cells, tissues, organs, plant parts or whole plants containing the construct.

Many examples of suitable marker genes are known in the art and can be used in screening and/or selection schemes. Reagents such as appropriate components of selection media are also known in the art. Examples of such marker genes include, but are not limited to, phosphomannose isomerase, phosphinothricin, neomycin phosphotransferase, hygromyci phosphotransferase, enolpyruvoyl-shikimate-3-phosphate synthetase, etc.

For example, phosphomannose isomerase (PMI) catalyses the interconversion of mannose 6-phosphate and fructose 6-phosphate in prokaryotic and eukaryotic cells. After uptake, mannose is phosphorylated by endogenous hexokinases to mannose-6-phosphate. Accumulation of mannose-6-phosphate leads to a block in glycolysis by inhibition of phosphoglucose-isomerase, resulting in severe growth inhibition. Phosphomannose-isomerase is encoded by the manA gene from Escherichia coli and catalyzes the conversion of mannose-6-phosphate to fructose-6-phosphate, an intermediate of glycolysis. On media containing mannose, manA expression in transformed plant cells relieves the growth inhibiting effect of mannose-6-phosphate accumulation and permits utilization of mannose as a source of carbon and energy, allowing transformed cells to grow.

Reporter proteins (such as GUS (β-glucuronidase), green fluorescent protein and derivatives thereof, and luciferase). Reporter genes may allow easy visual detection of transformed cells by visual screening and may also be used as marker genes. Non-limiting examples of eporter proteins include GUS (a β-glucuronidase), green fluorescent protein and derivatives thereof, and luciferase.

In some embodiments, the marker confers benefit(s) to the transgenic plant such as herbicide resistance, insect resistance, disease resistance, and increased tolerance to environmental stress (e.g., drought). (See, for example, the section on genes of interest above for an expanded discussion of some of these genes.)

Alternatively or additionally, a marker gene can provide some other visibly reactive response (e.g., may cause a distinctive appearance such as color or growth pattern relative to plants or plant cells not expressing the selectable marker gene in the presence of some substance, either as applied directly to the plant or plant cells or as present in the plant or plant cell growth media). It is now well known in the art that transcriptional activators of anthocyanin biosynthesis, operably linked to a suitable promoter in a construct, have widespread utility as non-phytotoxic markers for plant cell transformation.

B. Tissue-Specific and/or Tissue-Preferred Expression

In certain embodiments, heterologous gene products) is/are targeted to specific tissues of the transgenic plant such that the heterologous gene products) is/are present in only some plant tissues during the life of the plant. For example, tissue specific expression may be performed to preferentially express polypeptides encoded by heterologous genes in leaves and stems rather than grain or seed (which can reduce concerns about human consumption of genetically modified organism (GMOs)). Tissue-specific expression has other benefits including targeted expression of enzyme polypeptide(s) to the appropriate substrate.

In certain embodiments, heterologous gene product(s) is/are preferentiallly expressed certain tissues of the transgenic plant such that the heterologous gene products) is/are present at higher levels in some plant tissues than in others during the life of the plant.

Tissue-specific and/or tissue-preferred expression may be functionally accomplished by using one or more tissue-specific and/or tissue-preferred gene regulatory elements, such as some of the poplar promoters disclosed herein. A number of known tissue-specific promoters may be used in combination with gene regulatory elements disclosed herein. For example, in embodiments wherein two heterologous gene products are expressed in the same plant or other organism, expression of one heterologous gene product may be driven by a gene regulatory element from poplar as disclosed herein, while expression of the other heterologous gene product may be driven by a gene regulatory element that is known, such as a known tissue-specific promoter. Several tissue-specific regulated genes and/or promoters have been reported in plants. Some reported tissue-specific genes include without limitation genes encoding seed storage proteins (such as napin, cruciferin, β-conglycinin, and phaseolin), genes encoding zein or oil body proteins (such as oleosin), genes involved in fatty acid biosynthesis (including acyl carrier protein, stearoyl-ACP desaturase, and fatty acid desaturases (fad 2-1)), and other genes expressed during embryo development (such as Bce4 (Kridl et al., Seed Science Research, 1991, 1: 209)). Examples of tissue-specific promoters that have been described in the art include the lectin (Vodkin, Prog. Clin. Biol. Res., 1983, 138: 87; Lindstrom et al., Der. Genet., 1990, 11: 160), corn alcohol dehydrogenase 1 (Dennis et al., Nucleic Acids Res., 1984, 12: 983), corn light harvesting complex (Bansal et al., Proc. Natl. Acad. Sci. USA, 1992, 89: 3654), corn heat shock protein, pea small subunit RuBP carboxylase, Ti plasmid mannopine synthase, Ti plasmid nopaline synthase, petunia chalcone isomerase (van Tunen et al., EMBO J., 1988, 7:125), bean glycine rich protein 1 (Keller et al., Genes Dev., 1989, 3: 1639), truncated CaMV 35s (Odell et al., Nature, 1985, 313: 810), potato patatin (Wenzler et al., Plant Mol. Biol., 1989, 13: 347), root cell (Yamamoto et al., Nucleic Acids Res., 1990, 18: 7449), maize zein (Reina et al., Nucleic Acids Res., 1990, 18: 6425; Kriz et al., Mol. Gen. Genet., 1987, 207: 90; Wandelt et al., Nucleic Acids Res., 1989, 17 2354), PEPCase, R gene complex-associated promoters (Chandler et al., Plant Cell, 1989, 1: 1175), and chalcone synthase promoters (Franken et al., EMBO J., 1991, 10: 2605). Particularly useful for seed-specific expression is the pea vicilin promoter (Czako et al., Mol. Gen. Genet., 1992, 235: 33).

Tissue-specific and/or tissue-preferred expression may also be functionally accomplished by introducing a constitutively expressed gene in combination with an antisense gene that is expressed only in those tissues where the gene product is not desired, or where it is desired that the gene be expressed at lower levels. For example, a gene encoding an heterologous or homologous polypeptide may be expressed in all tissues under the control of a constitutive promoter such as constitutive poplar promoters disclosed herein and/or a known constitutive promoter such as the 35S promoter from Cauliflower Mosaic Virus. Expression of an antisense transcript of the gene in a particular tissue, using for example tissue-specific promoter or tissue-preferred promoter, would prevent accumulation of the enzyme polypeptide in that tissue. A tissue-specific and tissue-preferred poplar promoter disclosed herein and/or a known tissue-specific or tissue-preferred promoter may be used to drive expression of the antinsense transcript. For example, an antisense transcript of the gene for which tissue-specific or tissue-preferred expression is desired may be expressed in maize kernel using a zein promoter, thereby preventing accumulation of the gene product in seed. Hence the polypeptide encoded by the heterologous gene would be present in all tissues except the kernel.

### C. Subcellular-Specific Expression

In certain embodiments, heterologous gene product(s) is/are targeted to specific cellular compartments or organelles, such as, for example, the cytosol, the vacuole, the nucleus, the endoplasmic reticulum, the cell wall, the mitochondria, the apoplast, the peroxisomes, plastids, or combinations thereof. In some embodiments of the invention, the heterologous gene is expressed in one or more subcellular compartments or organelles, for example, the cell wall and/or endoplasmic reticulum, during the life of the plant.

In some embodiments, directing the product (e.g., a polypeptide and/or RNA molecule) of the heterologous gene to a specific cell compartment or organelle allows the product to be localized such that it will not come into contact with another molecule until desired. For example, if the product is an enzyme polypeptide, it may be possible to prevent the enzyme polypeptide from coming into contact with its substrate during plant growth. Thus, the enzyme polypeptide would not act until it is allowed to contact its substrate, e.g., following physical disruption of cell integrity by milling.

As another example, targeting expression of a cell wall-modifying and/or lignocellulolytic enzyme polypeptide to the cell wall (as in the apoplast) can help overcome the difficulty of mixing hydrophobic cellulose and hydrophilic enzymes that make it hard to achieve efficient hydrolysis with external enzymes.

In some embodiments, gene products are targeted to more than one subcellular compartments or organelles. Such targeting may allow one to increase the total amount of heterologous gene product in the plant. In some embodiments, targeting to one or more subcellular compartments or organelles is achieved using a gene regulatory element (such as a promoter) that drives expression specifically or preferentially in one or more subcellular compartments or organelles. Thus, for example, using an apoplast promoter with the E1 endo-1,4-β-glucanase gene and a chloroplast promoter with the E1 gene in a plant would increase total production of E1 compared to a single promoter/E1 construct in the plant.

Furthermore, in the case of expression of enzyme polypeptides that modify the cell wall (e.g., cell wall-modifying enzyme polypeptides and/or lignocellulolytic enzyme polypeptides)) one can minimize in vivo (pre-processing) deconstruction of the cell wall that occurs when multiple synergistic enzymes are present in a cell by using promoters targeted to different locations in the plant. For example, combining an endoglucanase with an apoplast promoter, a hemicellulase with a vacuole promoter, and an exoglucanase with a chloroplast promoter, sequesters each enzyme in a different part of the cell and achieves the advantages listed above. This method circumvents the limit on polypeptide or other heterologous gene product mass that can be expressed in a single organelle or location of the cell.

Localization of a nuclear-encoded protein (e.g., enzyme polypeptide) within the cell is known to be determined by the amino acid sequence of the protein. Protein localization can be altered, for example, by modifying the nucleotide sequence that encodes the protein in such a manner as to alter the protein's amino acid sequence. Polynucleotide sequences encoding polypeptides can be altered to redirect cellular localization of the encoded polypeptides by any suitable method (see, e.g., Dai et al., Trans. Res., 2005, 14: 627, the entire contents of which are herein incorporated by reference). In some embodiments of the invention, polypeptide localization is altered by fusing a sequence encoding a signal peptide to the sequence encoding the polypeptide. Signal peptides that may be used in accordance with the invention include without limitation a secretion signal from sea anemone equistatin (which allows localization to apoplasts) and secretion signals comprising the KDEL motif (which allows localization to endoplasmic reticulum).

### D. Expression Vectors

Generally, any vector that can be used constructed to express a product (e.g., polypeptide or RNA molecule) of a gene after introduction of such a vector in a host cell is considered an “expression vector.” Expression vectors typically contain nucleic acid constructs such as expression cassettes described above inserted into a vector. Expression vectors can be designed for expressing a gene product in any of a variety of host cells, including both prokaryotic (e.g., bacteria such as E. coli and Agrobacterium) and eukaryotic (e.g. insect, yeast (such as S. cerevisiae), and mammalian cells) host cells.

Nucleic acid constructs according to the present invention may be cloned into any of a variety of vectors, such as binary vectors, viral vectors, phage, phagemids, cosmids, and plasmids. Vectors suitable for transforming plant cells include, but are not limited to, Ti plasmids from Agrobacterium tumefaciens (J. Darnell, H. F. Lodish and D. Baltimore, “Molecular Cell Biology”, 2nd Ed., 1990, Scientific American Books: New York); plasmid containing a β glucuronidase gene and a cauliflower mosaic virus (CaMV) promoter plus a leader sequence from alfalfa mosaic virus (J. C. Sanford et al., Plant Mol. Biol. 1993, 22: 751-765); and plasmids containing a bar gene cloned downstream from a CaMV 35S promoter and a tobacco mosaic virus (TMV) leader. Other plasmids may additionally contain introns, such as that derived from alcohol dehydrogenase (Adh1) and/or other DNA sequences. The size of the vector is not a limiting factor.

For constructs that are intended be used in Agrobacterium-mediated transformation, the plasmid may contain an origin of replication that allows it to replicate in Agrobacterium and a high copy number origin of replication functional in E. coli. This permits facile production and testing of transgenes in E. coli prior to transfer to Agrobacterium for subsequent introduction in plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

Methods of preparation of nucleic acid constructs and expression vectors are well known in the art and can be found described in several textbooks such as, for example, J. Sambrook, E. F. Fritsch and T. Maniatis, “Molecular Cloning: A Laboratory Manual”, 1989, Cold Spring Harbor Laboratory: Cold Spring Harbor, and T. J. Silhavy, M. L. Berman, and L. W. Enquist, “Experiments with Gene Fusions”, 1984, Cold Spring Harbor Laboratory Cold Spring Harbor; F. M. Ausubel et al., “Current Protocols in Molecular Biology”, 1989, John Wiley & Sons: New York.

### II. Transgenic Plants

In one aspect, the present invention provides novel transgenic plants that express one or more polypeptides or RNA molecules under the control of a gene regulatory element provided by the present disclosure. The polypeptides or RNA molecules may be any polypeptide or RNA molecule for which expression in a plant is desired, including, but not limited to, those described herein.

In certain embodiments, provided are transgenic plants, the genomes of which are augmented with a recombinant polynucleotide comprising a gene regulatory element from poplaras described herein. In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO: 1 to 158. In some embodiments, the nucleotide sequence of the gene regulatory element is one of SEQ ID NO: 1 to 158. In some embodiments, the nucleotide sequence of the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO:90, 94, 103, 117, 131, 137, 145, and 158. (See, e.g., Examples 2, 3, 4, and 5). In some embodiments, the gene regulatory element has at least 65%, at least 70%, at least 75%, at least 80%, at least 85%, at least 90%, at least 95%, at least 96%, at least 97%, at least 98%, at least 99%, or more identity to at least one of SEQ ID NO: 90 and 103.

In some embodiments, the transgenic plant further comprises a heterologous gene operably linked to the gene regulatory element. In some such embodiments, the gene regulatory element regulates expression of the heterologous gene.

The heterologous gene may encode any polypeptide or RNA molecule for which expression in a plant is desired, including, but not limited to, those described herein. In some embodiments, the recombinant polynucleotide further comprises a gene terminator sequence that is operably linked to the heterologous gene.

Nucleic acid constructs, such as those described above, can be used to transform any plant. In some embodiments, plants are green field plants. In some embodiments, plants are grown specifically for “biomass energy” and/or phytoremediation.

In some embodiments, the plants are monocotyledonous plants. Examples of monocotyledonous plants that may be transformed in accordance with the practice of the present invention include, but are not limited to, bamboo, barley, maize (corn), millet, miscanthus, rice, rye, sorghum, sugarcane, switchgrass, turfgrass, and wheat. Alternatively or additionally, any grass species may be used.

In some embodiments, the plants are dicotyledonous plants. Examples of dicotyledonous plants that may be transformed in accordance with the practice of the present invention include, but are not limited to, alfalfa, Arabidopsis, aspen, birch, eucalyptus, flax, canola, cotton, cottonwood (e.g., Populus deltoides), hemlock, hemp, larch, oil seed rape, potato, poplar, sisal, spruce, soybean, sunflower, sweetgum, tobacco, tomato, and willow. Alternatively or additionally, any tree species may be used.

In some embodiments, the plants is a multicotyledonous plant. A non-limiting example of a multicotyledonous plant that may be transformed in accordance with the practice of the present invention is a pine tree (pinus sp.).

In some embodiments, the plant is a monocotyledonous or dicotyledonous plant of a genus selected from the group consisting of Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

In some embodiments, the plant is a monocotyledonous or dictoyledonous plant of a species selected from the group consisting of Abelmoschus esculentus (okra), Abies spp. (fir), Acer spp. (maple), Agrostis spp. (bentgrass), Allium cepa (onion), Alstroemeria spp., Ananas comosus (pineapple), Andropogon gerardii (big bluestem), Andrographis paniculata, Artemisia annua, Arundo donax (giant reed), Atropa belladonna, bamboo, Berberis spp., Beta vulgaris (sugarbeet), Bixa orellana, Brassica juncea, Brassica napus (canola), Brassica oleracea (broccoli, cauliflower, brussel sprouts), Calendula officinalis, Camellia sinensis (tea), Camptotheca acuminate, Cannabis sativa, Capsicum annum (hot and sweet pepper), Carthamus tinctorius (safflower), Catharanthus roseus, Cephalotaxus spp., Chrysanthemum parthenium, Cinchona officinalis, Citrullus lanatus (watermelon), Coffea arabica (coffee), Colchicum autumnale, Coleus forskohlii, Cucumis melo (melon), Cucumis sativus (cucumber), Cucurbita maxima (squash), Cucurbita moschata (squash), Cynodon dactylon (bermudagrass), Datura stomonium, Dianthus caryophyllus (carnation), Digitalis lanata, Digitalis purpurea, Dioscorea spp., Elaeis guineensis (palm), Ephedra sinica, Ephedra spp., Erianthus spp., Erythroxylum coca, Eucalyptus spp. (eucalyptus), Fragaria ananassa (strawberry), Festuca arundinacea (tall fescue), Galanthus wornorii, Glycine max (soybean), Gossypium hirsutum (cotton), Helianthus annuus (sunflower), Hevea spp. (rubber), Hordeum vulgare (barley), Hyoscyamus spp., Jatropha curcas (Jatropha), Lactuca sativa (lettuce), Linum usitatissimum (flax), Lolium spp. (ryegrass), Lupinus albus (lupin), Lycopersicon esculentum (tomato), Lycopodium serratum (Huperzia serrata), Lycopodium spp., Manihot esculenta (cassaya), Medicago sativa (alfalfa), Mentha spicata (mint), Menthapiperita (mint), Miscanthus giganteus (miscanthus), Miscanthus spp., Musa paradisiaca (banana), Nicotiana tabacum (tobacco), Oryza sativa (rice), Panicum spp., Panicum virgatum (switchgrass), Papaver somniferum (opium poppy), Papaver orientale, Parthenium argentatum (guayule), Pennisetum glaucum (pearl millet), Pennisetum purpureum (elephant grass), Petunia spp. (petunia), Phalaris arundinacea (reed canarygrass), Phleum pratense (timothy), Pinus spp. (pine), Poa pratensis (bluegrass), Poinsettia pulcherrima (poinsettia), Populus balsamifera (poplar), Populus spp., Populus tremuloides (aspen), Rosa spp. (rose), Saccharum sp. (energycane), Salix spp. (willow), Sanguinaria canadensis, Scopolia spp., Secale cereale (rye), Solanum melongena (eggplant), Solanum tuberosum (potato), Sorghum spp., Sorghum bicolor (sorghum, sudangrass), Spartina pectinata (prairie cord-grass), Spinacea oleracea (spinach), Rauwolfia serpentina, Rauwolfia spp., Ricinus communis (castor), Tanacetum parthenium, Taxus baccata, Taxus brevifolia, Theobroma cacao (cocoa), Triticosecale (triticum-wheat X rye), Triticum aestivum (wheat), Uniola paniculata (oats), Veratrum californica, Vinca rosea, Vitis vinifera (grape), and Zea mays (corn).

In some embodiments, the transgenic plant is fertile. In some embodiments, the transgenic plant is not fertile (i.e., sterile).

Using transformation methods, genetically modified plants, plant cells, plant tissue, seeds, and the like can be obtained.

Transformation according to the present invention may be performed by any suitable method. In certain embodiments, transformation comprises steps of introducing a nucleic acid construct, as described above, into a plant cell or protoplast to obtain a stably transformed plant cell or protoplast; and regenerating a whole plant from the stably transformed plant cell or protoplast.

Cell Transformation

Delivery or introduction of a nucleic acid construct into eukaryotic cells may be accomplished using any of a variety of methods. The choice of a particular method used for the transformation is not critical to the instant invention. Suitable techniques include, but are not limited to, non-biological methods, such as microinjection, microprojectile bombardment, electroporation, induced uptake, and aerosol beam injection, as well as biological methods such as direct DNA uptake, liposome-mediated transfection, polyethylene glycol-mediated transfection, and Agrobacterium-mediated transformation. Any combinations of the above methods that provide for efficient transformation of plant cells or protoplasts may also be used in the practice of the invention.

Methods of introduction of nucleic acid constructs into plant cells or protoplasts have been described. See, for example, “Methods for Plant Molecular Biology”, Weissbach and Weissbach (Eds.), 1989, Academic Press, Inc; “Plant Cell, Tissue and Organ Culture: Fundamental Methods”, 1995, Springer-Verlag: Berlin, Germany; and U.S. Pat. Nos. 4,945,050; 5,036,006; 5,100,792; 5,240,855; 5,302,523; 5,322,783; 5,324,646; 5,384,253; 5,464,765; 5,538,877; 5,538,880; 5,550,318; 5,563,055; and 5,591,616).

In particular, electroporation has frequently been used to transform plant cells (see, for example, U.S. Pat. No. 5,384,253). This method is generally performed using friable tissues (such as a suspension culture of cells or embryogenic callus) or target recipient cells from immature embryos or other organized tissue that have been rendered more susceptible to transformation by electroporation by exposing them to pectin-degrading enzymes or by mechanically wounding them in a controlled manner Intact cells of maize (see, for example, K. D'Halluin et al., Plant cell, 1992, 4: 1495-1505; C. A. Rhodes et al., Methods Mol. Biol. 1995, 55: 121-131; and U.S. Pat. No. 5,384,253), wheat, tomato, soybean, and tobacco have been transformed by electroporation. As reviewed, for example, by G. W. Bates (Methods Mol. Biol. 1999, 111: 359-366), electroporation can also be used to transform protoplasts.

Another method of transformation is microprojectile bombardment (e.g., through use of a “gene gun”) (see, for example, U.S. Pat. Nos. 5,538,880; 5,550,318; and 5,610,042; and WO 94/09699). In this method, nucleic acids are delivered to living cells by coating or precipitating the nucleic acids onto a particle or microprojectile (for example tungsten, platinum or gold), and propelling the coated microprojectile into the living cell. Microprojectile bombardment techniques are widely applicable, and may be used to transform virtually any monocotyledonous or dicotyledonous plant species (see, for example, U.S. Pat. Nos. 5,036,006; 5,302,523; 5,322,783 and 5,563,055; WO 95/06128; A. Ritala et al., Plant Mol. Biol. 1994, 24: 317-325; L. A. Hengens et al., Plant Mol. Biol. 1993, 23: 643-669; L. A. Hengens et al., Plant Mol. Biol. 1993, 22: 1101-1127; C. M. Buising and R. M. Benbow, Mol. Gen. Genet. 1994, 243: 71-81; C. Singsit et al., Transgenic Res. 1997, 6: 169-176).

The use of Agrobacterium-mediated transformation of plant cells is well known in the art (see, for example, U.S. Pat. No. 5,563,055). This method has long been used in the transformation of dicotyledonous plants, including Arabidopsis and tobacco, and has recently also become applicable to monocotyledonous plants, such as rice, wheat, barley and maize (see, for example, U.S. Pat. No. 5,591,616). In plant strains where Agrobacterium-mediated transformation is efficient, it is often the method of choice because of the facile and defined nature of the gene transfer. In some embodiments, Agrobacterium-mediated transformation of plant cells is carried out in two phases. First, the steps of cloning and DNA modifications are performed in E. coli, and then the plasmid containing the gene construct of interest is transferred by heat shock treatment into Agrobacterium, and the resulting Agrobacterium strain is used to transform plant cells. In some embodiments, Agrobacterium infiltrates plant leaves. In some embodiments, the bacterial strain Agrobacterium tumefaciens is used to transform plant cells.

Transformation of plant protoplasts can be achieved using methods based on calcium phosphate precipitation, polyethylene glycol treatment, electroporation, and combinations of these treatments (see, e.g., I. Potrykus et al., Mol. Gen. Genet. 1985, 199: 169-177; M. E. Fromm et al., Nature, 1986, 31: 791-793; J. Callis et al., Genes Dev. 1987, 1: 1183-1200; S. Omirulleh et al., Plant Mol. Biol. 1993, 21: 415-428).

Alternative methods of plant cell transformation, that have been reviewed, for example, by M. Rakoczy-Trojanowska (Cell Mol. Biol. Lett. 2002, 7: 849-858; the contents of which are herein incorporated by reference in their entirety), can also be used in the practice of the present invention.

In some embodiments, successful delivery of the nucleic acid construct into the host plant cell or protoplast is preliminarily evaluated visually. Selection of stably transformed plant cells can be performed, for example, by introducing into the cell a nucleic acid construct comprising a marker gene which confers resistance to some normally inhibitory agent, such as an antibiotic or herbicide. Examples of antibiotics that may be used include aminoglycoside antibiotics (such as neomycin, kanamycin, and paromomycin) and the antibiotic hygromycin. Several aminoglycoside phosphotransferases confer resistance to aminoglycoside antibiotics, and include aminoglycoside phosphotransferase I (aph-I) enzyme and aminoglycoside (or neomycin) phosphotransferase II (APH-II or NPTII), which, though unrelated, both have ability to inactivate the antibiotic G418. The hygromycin phosphotransferase (denoted hpt, hph or aphIV) gene was originally derived from Escherichia coli. Hygromycin phosphotransferase (HPT) detoxifies the aminocyclitol antibiotic hygromycin B. As is known in the art, plants have been transformed with the hpt gene, and hygromycin B has proved very effective in the selection of a wide range of plants.

Examples of herbicides that may be used include phosphinothricin and glyphosate. Potentially transformed cells then are exposed to the selective agent. Cells where the resistance-conferring gene has been integrated and expressed at sufficient levels to permit cell survival will generally be present in the population of surviving cells.

Alternatively or additionally, host cells comprising a nucleic acid sequence of the invention and expressing a gene product encoding by inventive nucleic acids may be identified and selected by a variety of procedures, including, but not limited to, DNA-DNA or DNA-RNA hybridization and protein bioassay or immunoassay techniques such as membrane, solution, or chip-based technologies for the detection and/or quantification of nucleic acids or proteins.

Plant cells are available from a wide range of sources including the American Type Culture Collection (Rockland, Md.), or from any of a number of seed companies including, for example, A. Atlee Burpee Seed Co. (Warminster, Pa.), Park Seed Co. (Greenwood, S.C.), Johnny Seed Co. (Albion, Me.), or Northrup King Seeds (Hartsville, S.C.). Descriptions and sources of useful host cells can be found in I. K. Vasil, “Cell Culture and Somatic Cell Genetics of Plants”, Vol. I, II and II; 1984, Laboratory Procedures and Their Applications Academic Press: New York; R. A. Dixon et al., “Plant Cell Culture—A Practical Approach”, 1985, IRL Press: Oxford University; and Green et al., “Plant Tissue and Cell Culture”, 1987, Academic Press: New York.

Plant cells or protoplasts stably transformed according to the present invention are provided herein.

Plant Regeneration

In plants, every cell is capable of regenerating into a mature plant and contributing to the germ line such that subsequent generations of the plant will contain the transgene of interest. Stably transformed cells may be grown into plants according to conventional ways (see, for example, McCormick et al., Plant Cell Reports, 1986, 5: 81-84). Plant regeneration from cultured protoplasts has been described, for example by Evans et al., “Handbook of Plant Cell Cultures”, Vol. 1, 1983, MacMilan Publishing Co: New York; and I. R. Vasil (Ed.), “Cell Culture and Somatic Cell Genetics of Plants”, Vol. I (1984) and Vol. II (1986), Acad. Press: Orlando.

Means for regeneration vary from species to species of plants, but generally a suspension of transformed protoplasts or a Petri plate containing transformed explants is first provided. Callus tissue is formed and shoots may be induced from callus and subsequently roots. Alternatively, somatic embryo formation can be induced in the callus tissue. These somatic embryos germinate as natural embryos to form plants. The culture media will generally contain various amino acids and plant hormones, such as auxin and cytokinins. Glutamic acid and proline may also be added to the medium. Efficient regeneration generally depends on the medium, on the genotype, and on the history of the culture.

Regeneration from transformed individual cells to obtain transgenic whole plants has been shown to be possible for a large number of plants. For example, regeneration has been demonstrated for dicots (such as apple; Malus pumila; blackberry, Rubus; Blackberry/raspberry hybrid, Rubus; red raspberry, Rubus; carrot; Daucus carota; cauliflower; Brassica oleracea; celery; Apium graveolens; cucumber; Cucumis sativus; eggplant; Solanum melongena; lettuce; Lactuca sativa; potato; Solanum tuberosum; rape; Brassica napus; soybean (wild); Glycine canescens; strawberry; Fragaria×ananassa; tomato; Lycopersicon esculentum; walnut; Juglans regia; melon; Cucumis melo; grape; Vitis vinifera; and mango; Mangifera indica) as well as for monocots (such as rice; Oryza sativa; rye, Secale cereale; and Maize).

Primary transgenic plants may then be grown using conventional methods. Various techniques for plant cultivation are well known in the art. Plants can be grown in soil, or alternatively can be grown hydroponically (see, for example, U.S. Pat. Nos. 5,364,451; 5,393,426; and 5,785,735). Primary transgenic plants may be either pollinated with the same transformed strain or with a different strain and the resulting hybrid having the desired phenotypic characteristics identified and selected. Two or more generations may be grown to ensure that the subject phenotypic characteristics is stably maintained and inherited and then seeds are harvested to ensure that the desired phenotype or other property has been achieved.

As is well known in the art, plants may be grown in different media such as soil, growth solution or water.

Selection of plants that have been transformed with the construct may be performed by any suitable method, for example, with northern blot, Southern blot, herbicide resistance screening, antibiotic resistance screening or any combinations of these or other methods. The Southern blot and northern blot techniques, which test for the presence (in a tissue such as a plant tissue) of a nucleic acid sequence of interest and of its corresponding RNA, respectively, are standard methods (see, for example, Sambrook & Russell, “Molecular Cloning”, 2001, Cold Spring Harbor Laboratory Press: Cold Spring Harbor).

### III. Uses of Inventive Transgenic Plants

Transgenic plants and plant parts disclosed herein may be used advantageously in a variety of applications. In many embodiments, transgenic plants of the present invention express polypeptides that confer desirable traits to the plant and/or plant biomass (e.g., resistance to herbicides, resistance to environmental stress, resistance to pests and diseases). In some embodiments, expression of such polypeptides results in downstream process innovations and/or improvements in a variety of applications including ethanol production, phytoremediation and hydrogen production.

### A. Ethanol Production

In some embodiments, plants transformed according to the present invention provide a means of increasing ethanol yields, reducing pretreatment costs by reducing acid/heat pretreatment requirements for saccharification of biomass; and/or reducing other plant production and processing costs, such as by allowing multi-applications and isolation of commercially valuable by-products. For example, a gene regulatory element provided by the present disclosure may drive expression of one or more lignocellulolytic enzyme polypeptide(s) and/or cell wall modifying enzyme polypeptide(s) in a transgenic plant and such enzyme polypeptides may allow biomass from the transgenic plant to be processed to produce more easily and/or cost effectively.

Plant Culture

Farmers can grow different transgenic plants of the present invention (e.g., different variety of transgenic corn, each expressing a transgenic polypeptide or RNA) simultaneously, achieving the desired “blend” of gene products produced by changing the seed ratio.

Plant Harvest

Transgenic plants of the present invention can be harvested as known in the art. For example, current techniques may cut corn stover at the same time as the grain is harvested, but leave the stover lying in the field for later collection. However, dirt collected by the stover can interfere with ethanol production from lignocellulosic material. The present invention provides a method in which transgenic plants are cut, collected, stored, and transported so as to minimize soil contact. In addition to minimizing interference from dirt with ethanol production, this method can result in reduction in harvest and transportation costs.

Tempering

In some embodiments, provided transgenic plants undergo a tempering phase that conditions the biomass for pretreatment and hydrolysis. Tempering may facilitate reducing severity of pretreatment conditions to achieve a desired glucan conversion yield and/or improving hydrolysis and glucan conversion after treatment. For example, a typical yield from biomass that has been pretreated under standard pretreatment conditions (e.g., 1% sulfuric acid, 170° C., for 10 minutes) is at least 80% glucan conversion. When tempered as described herein, the same typical yield may be achieved under less severe pretreatment conditions and/or with reduced amounts of externally applied enzymes. Less severe pretreatment conditions may comprise, for example, reduced acid concentrations, lower incubation temperatures, and/or shorter pretreatment times.

In some embodiments, when tempered as described herein and using the same pretreatment conditions, typical yield may be increased above at least 80% glucan conversion.

Without wishing to be bound by any particular theory, tempering may facilitate such improvements by, for example, allowing activation of endoplant enzyme polypeptides after harvest, increasing susceptibility of lignin and hemicellulose to traditional pretreatment, and/or increasing accessibility of polysaccharides (e.g., cellulose).

A variety of techniques for tempering may be used. In some embodiments, tempering comprises increasing the temperature of the biomass to activate thermophilic enzymes. Increasing the temperature to activate thermophilic enzymes may be achieved, for example, by one or more of ensilement, grinding, pelleting, and warm water suspension/slurries. In some embodiments, tempering comprises disrupting cell walls. Cell wall disruption may be achieved, for example, by sonication and/or liquid extraction to release enzyme polypeptides from sequestered locations in the plant (which may allow further activation and/or extraction to be added back after pretreatment). In some embodiments, tempering comprises adding accessory enzyme polypeptides during an incubation period before pretreatment. Such accessory enzyme polypeptides may weaken cross linking and improve accessibilty of the biomass to embedded glucanases or xylanases. In some embodiments, tempering comprises incubating the biomass in a particular set of conditions (e.g., a particular temperature, particular pH, and/or particular moisture conditions). Such incubations may in some embodiments increase susceptibility to various glucanases and/or accessory enzyme polypeptides present in the plant tissues or added to the sample. For example, samples may be tempered as a liquid slurry (e.g., comprising about 10% to about 30% total solids) under conditions favorable to activate cell wall-modifying enzymes. In some embodiments, samples are tempered as a liquid slurry for about 1 to about 48 hours. In some embodiments, conditions favorable to activate cell wall-modifying enzymes comprise a pH of about 4 to about 7 and a temperature of about 25° C. to about 100° C. Alternatively or additionally, samples may be tempered as a lower moisture ensilement (e.g., about 40% to about 60% total solids) under anaerobic conditions. In some embodiments, samples are ensiled for about 21 days to several months.

In some embodiments, tempering is integrated with other processes such as one or more of harvest, storage, and transportation of biomass. For example, biomass can be ensiled under conditions that condition the biomass for subsequent pretreatment and hydrolysis; that is, storage and tempering are combined. In some embodiments, during ensilement of biomass, temperatures are increased in the ensiled material such that thermally active embedded enzymes are activated. Ensilement conditions may allow preservation of biomass while providing sufficient time for enzyme polypeptides to affect characteristics of the biomass (such as, for example, amenability to pretreatment and improvement of subsequent hydrolysis).

In some embodiments, the tempering phase precedes entirely the pretreatment phase. In some embodiments, the tempering phase overlaps with the pretreatment phase.

In some embodiments as described herein, transgenic plants express more than one cell wall-modifying enzyme polypeptide. In some such embodiments, it may be desirable to activate enzyme polypeptides sequentially. It may be desirable to do so, for example, if the efficiency of endoplant enzymes is a function of the sequence in which they are activated. For example, beta-glucosidases may be most efficient after endo- and exoglucanases have cleaved cellulose into dimers, and cellulases and hemicellulases may be more efficient when accessory enzymes have reduced cross-linkages between cellulose, hemicellulose, and lignin. Accordingly, in some embodiments, cellulases might be activated after ferulic acid esterases (FAEs) have had the opportunity to cleave ferulate-polysaccharide-lignin complexes, or after other accessory enzymes have had the opportunity to cleave cellulose-hemicellulose cross linkages.

Sequential activation could be attained, for example, by using enzymes with different peak temperature and/or pH optima. Increasing temperature continually or stepwise (e.g., during a tempering step), could thereby allow activation of enzyme polypeptides with lower temperature optima first. For example, a wound-induced promoter could be used to produce a non-thermostable enzyme polypeptide after harvesting that breaks lingin cross-links and leads to cell death, before increasing temperature during tempering to activate a thermostable cellulase in the biomass.

In some embodiments as described herein, cell wall-modifying enzyme polypeptides are specifically targeted to organelles and/or plant parts. In some embodiments, cell wall-modifying enzyme polypeptides are specifically targeted to seeds. Cell wall hydrolyzing enzymes in the grain could improve yields of fermentable sugars by targeting the cellulose and hemicelluolose in the grain bran and fiber, or could loosen or weaken the outer layers of the grain kernel, making it easier to mill. Starch in corn grain is often processed to produce ethanol, but significant quantities of cellulose and hemicellulose from the bran and fiber are not used. In some embodiments, incorporating a tempering step prior to starch hydrolysis (e.g., of transgenic corn grain), endogenous enzymes can act on the fiber and bran and increase the yield of fermentable sugars. In some embodiments, dry seed (e.g., dry wheat) is tempered by soaking in water at a slightly elevated temperature for several hours before further processing. Such a tempering step may decrease the energy required for milling and increase the quality and eventual yield. Endogenous enzymes in the grain may also provide additional benefits.

In some embodiments, tempering comprises externally applying an amount of at least one cell wall-modifying enzyme polypeptide. External application of cell wall-modifying enzyme polypeptides is discussed in more detail in the “Saccharification” section.

In some embodiments, the seed or grain of a transgenic plant is tempered.

Pretreatment

Conventional methods for processing plant biomass include physical, chemical, and/or biological pretreatments. For example, physical pretreatment techniques can include one or more of various types of milling, crushing, irradiation, steaming/steam explosion, and hydrothermolysis. Chemical pretreatment techniques can include acid, alkaline, organic solvent, ammonia, sulfur dioxide, carbon dioxide, and pH-controlled hydrothermolysis. Biological pretreatment techniques can involve applying lignin-solubilizing microorganisms (T.-A. Hsu, “Handbook on Bioethanol: Production and Utilization”, C. E. Wyman (Ed.), 1996, Taylor & Francis: Washington, D.C., 179-212; P. Ghosh and A. Singh, A., Adv. Appl. Microbiol., 1993, 39: 295-333; J. D. McMillan, in “Enzymatic Conversion of Biomass for Fuels Production”, M. Himmel et al., (Eds.), 1994, Chapter 15, ACS Symposium Series 566, American Chemical Society: B. Hahn-Hagerdal, Enz. Microb. Tech., 1996, 18: 312-331; and L. Vallander and K. E. L. Eriksson, Adv. Biochem. Eng./Biotechnol., 1990, 42: 63-95). The purpose of the pretreatment step is to break down the lignin and carbohydrate structure to make the cellulose fraction accessible to cellulolytic enzymes.

Simultaneous use of transgenic plants that express one or more enzyme polypeptides (e.g., lignocellulolytic enzyme polypeptides and/or cell wall-modifying enzyme polypeptides) according to the present invention may reduce or eliminate expensive grinding of the biomass and/or reduce or eliminate the need for heat and strong acid required to strip lignin and hemicellulose away from cellulose before hydrolyzing the cellulose.

In some embodiments, lignocellulosic biomass of plant parts obtained from inventive transgenic plants is more easily hydrolyzable than that of non-transgenic plants. Thus, the extent and/or severity of pretreatment required to achieve a particular level of hydrolysis is reduced. Therefore, the present invention in some embodiments provides improvements over existing pretreatment methods. Such improvements may include one or more of: reduction of biomass grinding, elimination of biomass grinding, reduction of the pretreatment temperature, elimination of heat in the pretreatment, reduction of the strength of acid in the pretreatment step, elimination of acid in the pretreatment step, and any combination thereof.

In some embodiments, lower temperatures of pretreatment may be used to achieve a desired level of hydrolysis. In some embodiments, pretreating is performed at temperatures below about 175° C., below about 145° C., or below about 115° C. For example, under some conditions, the yield of hydrolysis products from lignocellulosic biomass from transgenic plant parts pretreated at about 140° C. is comparable to the yield of hydrolysis products from non-transgenic plant parts pretreated at about 170° C. Under some conditions, the yield of hydrolysis products from lignocellulosic biomass from transgenic plant parts pretreated at about 170° C. is above about 60%, above about 70%, above about 80%, or above about 90% of theoretical yields. Under some conditions, the yield of hydrolysis products from lignocellulosic biomass from transgenic plant parts pretreated at about 140° C. is above about 60%, above about 70%, or above about 80% of theoretical yields. Under some conditions, the yield of hydrolysis products from lignocellulosic biomass from transgenic plant parts pretreated at about 110° C. is above about 40%, above about 50%, or above about 60% of theoretical yields. Such yields from transgenic plant parts can represent an increase of up to about 20% of yields from non-transgenic plant parts.

In some embodiments, such improvements are observed in inventive transgenic plants expressing an enzyme polypeptide (e.g., a cell wall-modifying enzyme polypeptide and/or lignocellulolytic enzyme polypeptide) at a level less than about 0.5%, less than about 0.4%, less than about 0.3%, less than about 0.2%, or less than about 0.1% of total soluble protein. Without wishing to be bound by any particular theory, the inventors propose that low levels of enzyme expression may facilitate modifying the cell wall, possibly by nicking cellulose or hemicellulose strands. Such modification of the cell wall may make the biomass more susceptible to pretreatment. Thus, biomass from inventive transgenic plants expressing low levels of cell wall-modifying enzymes may require less pretreatment, and/or pretreatment in less severe conditions.

In certain embodiments, the pretreated material is used for saccharification without further manipulation. In other embodiments, it is desired to process the plant tissue so as to produce an extract comprising the cell wall-modifying enzyme polypeptide(s). In this case, the extraction is carried out in the presence of components known in the art to favor extraction of active enzymes from plant tissue and/or to enhance the degradation of cell-wall polysaccharides in the lignocellulosic biomass. Such components include, but are not limited to, salts, chelators, detergents, antioxidants, polyvinylpyrrolidone (PVP), and polyvinylpolypyrrolidone (PVPP). The remaining plant tissue may then be submitted to a pretreatment process.

Saccharification

In saccharification (or enzymatic hydrolysis), lignocellulose is converted into fermentable sugars (i.e., glucose monomers) by enzyme polypeptides present in the pretreated material. If desired, externally applied cellulolytic enzyme polypeptides (i.e., enzymes not produced by the transgenic plants being processed) may be added to this mixture. Extracts comprising transgenically expressed enzyme polypeptides obtained as described above can be added back to the lignocellulosic biomass before saccharification. Here again, externally applied cellulolytic enzyme polypeptides may be added to the saccharification reaction mixture.

In some embodiments, the amount of externally applied enzyme polypeptide that is required to achieve a particular level of hydrolysis of lignocellulosic biomass from inventive transgenic plants is reduced as compared to the amount required to achieve a similar level of hydrolysis of lignocellulosic biomass from non-transgenic plants. For example, in some embodiments, processing transgenic lignocellulosic biomass in the presence of as low as 15 mg externally applied cellulase per gram of biomass (15 mg/g) yields a similar level of hydrolysis as processing non-transgenic lignocellulosic biomass in the presence of 100 mg/g cellulase. This represents a reduction of almost 90% of cellulases needed for hydrolysis can be achieved when processing biomass from inventive transgenic plants. Such a reduction in externally applied cellulases used can represent significant cost savings.

In some embodiments, a mixture of enzyme polypeptides each having different enzyme activities (e.g., exoglucanase, endoglucanase, hemi-cellulase, beta-glucosidase, and combinations thereof), and/or an enzyme polypeptide having more than one enzyme activity (e.g., exoglucanase, endoglucanase, hemi-cellulase, beta-glucosidase, and combinations thereof) is added during a “treatment” step to promote saccharification. Without wishing to be bound by any particular theory, such combinations of enzyme activity, whether through the activity of an enzyme complex or other mixture of enzymes, may allow a greater degree of hydrolysis than can be achieved with a single enzyme activity alone. Commercially available enzyme complexes that can be employed in the practice of the invention include, but are not limited to, Accellerase™ 1000 (Genencor), which contains multiple enzyme activities, mainly exoglucanase, endoglucanase, hemi-cellulase, and beta-glucosidase.

Saccharification is generally performed in stirred-tank reactors or fermentors under controlled pH, temperature, and mixing conditions. A saccharification step may last up to 200 hours. Saccharification may be carried out at temperatures from about 30° C. to about 65° C., in particular around 50° C., and at a pH in the range of between about 4 and about 5, in particular, around pH 4.5. Saccharification can be performed on the whole pretreated material.

The present Applicants had previously shown that adding cellulases to plants transgenically expressing E1, an endoglucanse (EC 3.2.1.4) increases total glucose production compared to adding cellulases to non-transgenic plants, which suggests that simply using transgenic E1 plants with current external cellulase techniques can substantially increase yields of products (e.g., such as ethanol, methanol, butanol, and/or other alcohols). The experiment also indicates that adding cellulases to E1 plants increases total glucose production compared to adding cellulases to non-transgenic plants. This is an important result since it suggests that simply using transgenic E1 plants with current external cellulase techniques can substantially increase yields of products (e.g., of ethanol, methanol, butanol, and/or other alcohols) in the presence or absence of pretreatment processes.

Fermentation

In the fermentation step, sugars, released from the lignocellulose as a result of the pretreatment and enzymatic hydrolysis steps, are fermented to one or more organic substances (e.g., ethanol, methanol, butanol, or other alcohols) by a fermenting microorganism, such as yeasts and/or bacteria. The fermentation can also be carried out simultaneously with the enzymatic hydrolysis in the same vessels, again under controlled pH, temperature and mixing conditions. When saccharification and fermentation are performed simultaneously in the same vessel, the process is generally termed simultaneous saccharification and fermentation or SSF.

Fermenting microorganisms and methods for their use inproduction (e.g., of ethanol, methanol, butanol, and/or other alcohols) are known in the art (Sheehan, “The Road to Bioethanol: A Strategic Perspective of the US Department of Energy's National Ethanol Program” In: “Glycosyl Hydrolases For Biomass Conversion”, ACS Symposium Series 769, 2001, American Chemical Society: Washington, D.C.). Existing ethanol production methods that utilize corn grain as the biomass typically involve the use of yeast, particularly strains of Saccharomyces cerevisiae. Such strains can be utilized in the methods of the invention. While such strains may be preferred for the production of alcohols (e.g., ethanol, methanol, and butanol) from glucose that is derived from the degradation of cellulose and/or starch, the methods of the present invention do not depend on the use of a particular microorganism, or of a strain thereof, or of any particular combination of said microorganisms and said strains.

Yeast or other microorganisms are typically added to the hydrolysate and the fermentation is allowed to proceed for 24-96 hours, such as 35-60 hours. The temperature of fermentation is typically between 26-40° C., such as 32° C., and at a pH between 3 and 6, such as about pH 4-5.

A fermentation stimulator may be used to further improve the fermentation process, in particular, the performance of the fermenting microorganism, such as, rate enhancement and yield (e.g., of ethanol, methanol, butanol, and/or other alcohols). Fermentation stimulators for growth include vitamins and minerals. Examples of vitamins include multivitamin, biotin, pantothenate, nicotinic acid, meso-inositol, thiamine, pyridoxine, para-aminobenzoic acid, folic acid, riboflavin, and vitamins A, B, C, D, and E (Alfenore et al., “Improving ethanol production and viability of Saccharomyces cerevisiae by a vitamin feeding strategy during fed-batch process”, 2002, Springer-Verlag). Examples of minerals include minerals and mineral salts that can supply nutrients comprising phosphate, potassium, manganese, sulfur, calcium, iron, zinc, magnesium and copper.

Recovery

Following fermentation (or SSF), the mash is distilled to extract the alcohol (e.g., ethanol, methanol, butanol, and/or other alcohols). Alcohol with a purity greater than 96 vol. % can be obtained.

By-Products

The hydrolysis process of lignocellulosic raw material also releases by-products such as weak acids, furans, and phenolic compounds, which are inhibitory to the fermentation process. Removing such by-products may enhance fermentation.

In some embodiments, processing of provided transgenic plants comprise removing, from the hydrolysate, products of the enzymatic process that cannot be fermented. Such products comprise, but are not limited to, lignin, lignin breakdown products, phenols, and furans. In certain embodiments, products of the enzymatic process that cannot be fermented are separated and used subsequently. For example, products can be burned to provide heat required in some steps of the alcohol (e.g., ethanol, methanol, butanol) production such as saccharification, fermentation, and alcohol (e.g., ethanol, methanol, butanol) distillation, thereby reducing costs by reducing the need for current external energy sources such as natural gas. Alternatively or additionally, such by-products may have commercial value. For example, phenols can find applications as chemical intermediates for a wide variety of applications, ranging from plastics to pharmaceuticals and agricultural chemicals. Phenol condensed to with aldehydes (e.g., methanol) make resinous compounds, which are the basis of plastics which are used in electrical equipment and as bonding agents in manufacturing wood products such as plywood and medium density fiberboard (MDF).

Separation of by-products from the hydrolysate can be done using a variety of chemical and physical techniques that rely on the different chemical and physical properties of the by-products (e.g., lignin and phenols). Such techniques include, but are not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, distillation, or extraction.

Some of the hydrolysis by-products, such as phenols, or fermentation/processing products, such as methanol, can be used as ethanol denaturants. Currently about 5% gasoline is added immediately to distilled ethanol as a denaturant under the Bureau of Alcohol, Tobacco and Firearms regulations, to prevent unauthorized non-fuel use. This requires shipping gasoline to the ethanol production plant, then shipping the gas back with the ethanol to the refinery. The gas also impedes the use of ethanol-optimized engines that make use of ethanol's higher compression ratio and higher octane to improve performance. Using transgenic plant derived phenols and/or methanol as denaturants in lieu of gasoline can reduce costs and increase automotive engine design alternatives.

Reducing Lignin Content

Another way of reducing lignin and lignin breakdown products that are not fermentable in hydrolysate is to reduce lignin content in a transgenic plant of the present invention. Such methods have been developed and can be used to modify the inventive plants (see, for example, U.S. Pat. Nos. 6,441,272 and 6,969,784, U.S. Pat. Appln. No. 2003-0172395, US and PCT publication No. WO 00/71670).

Combined Starch Hydrolysis and Cellulolytic Material Hydrolysis

Transgenic plants and plant parts disclosed herein can be used in methods involving combined hydrolysis of starch and of cellulosic material for increased yields (e.g, of ethanol, methanol, butanol, and/or other alcohols). In addition to providing enhanced yields, these methods can be performed in existing starch-based alcohol processing facilities.

Starch is a glucose polymer that is easily hydrolyzed to individual glucose molecules for fermentation. Starch hydrolysis may be performed in the presence of an amylolytic microorganism or enzymes such as amylase enzymes. In certain embodiments of the invention, starch hydrolysis is performed in the presence of at least one amylase enzyme. Examples of suitable amylase enzymes include α-amylase (which randomly cleaves the α(1-4)glycosidic linkages of amylose to yield dextrin, maltose or glucose molecules) and glucoamylase (which cleaves the α(1-4) and α(1-6)glycosidic linkages of amylose and amylopectin to yield glucose).

Hydrolysis of starch and hydrolysis of cellulosic material from provided transgenic plants can be performed simultaneously (i.e., at the same time) under identical conditions (e.g., under conditions commonly used for starch hydrolysis). Alternatively, the hydrolytic reactions can be performed sequentially (e.g., hydrolysis of lignocellulose can be performed prior to hydrolysis of starch). When starch and cellulosic material are hydrolyzed simultaneously, the conditions are preferably selected to promote starch degradation and to activate cell wall-modifying enzyme polypeptide(s) for the degradation of lignocellulose. Factors that can be varied to optimize such conditions include physical processing of the plants or plant parts, and reaction conditions such as pH, temperature, viscosity, processing times, and addition of amylase enzymes for starch hydrolysis.

Provided transgenic plants (or plant parts) may be used alone or in a mixture with non-transgenic plants (or plant parts). Suitable plants include any plants that can be employed in starch-based alcohol production (e.g., corn, wheat, potato, cassaya, etc.). For example, the present inventive methods may be used to increase ethanol yields from corn grains.

### EXAMPLES

The following examples describe some of the preferred modes of making and practicing the present invention. However, it should be understood that these examples are for illustrative purposes only and are not meant to limit the scope of the invention.

### Example 1

### Identification and Isolation of Poplar Promoters

In the present example, gene regulatory elements were identified and isolated from the poplar genome. Promoters of poplar genes were identified by searching for gene sequences similar to that of genes having or suspected of having desirable expression patterns in other plants. Nucleic acids containing identified promoters were isolated by polymerase chain reaction (PCR)-based amplification. These gene regulatory elements may be useful, for example, in driving expression of genes in transgenic plants.

### Identification of Poplar Gene Regulatory Elements

Two methods were used to identify gene regulatory elements of interest from the poplar genome. In one method, protein sequences of interest were identified in the poplar proteome (see the website whose address is “http://” followed immediately by “genome.jgi-psf.org/Poptr1_{—}1/Poptr1_{—}1.home.html”) using the BLASTp tool by searching for sequences similar to protein sequences of Arabidopsis or maize gene products having functions and/or expression patterns of interest. Protein sequences of gene products from Arabidopsis or maize that are known to be involved in cell structure and function, intermediary metabolism, or tissue-specific and developmental stage-specific functions were used as input in the BLASTp searches. Genomic DNA sequences that encode the putative homologous proteins were then identified.

In a second method, publicly available EST databases were analyzed for the abundance of particular transcripts in various tissues and corresponding full length genes were identified in the poplar genome.

Both approaches resulted in identification of poplar genomic DNA sequences of interest, and novel regulatory DNA sequences within and/or in the proximity of such genomic DNA sequences were discovered for several classes of genes. The 3 kb region upstream of the genes were identified using a poplar genome database and designated as regulatory DNA sequences.

These novel regulatory DNA sequences are listed as SEQ ID NO: 1 through SEQ ID NO: 158 of the accompanying Sequence Listing.

### Isolation and Cloning of Poplar Gene Regulatory Elements

Using genomic information obtained as described above, oligonucleotides (see Table 2) were designed for use as PCR primers to amplify various poplar gene regulatory elements. Poplar promoter sequences were amplified with high-fidelity Phusion Taq Polymerase (New England Biolabs, MA) or PLANTAMP™ PCR system (Epicentre Biotechnologies WI) using genomic DNA isolated from young leaves of two month old hybrid poplar plants (Poplulus tremula×P. alba, clone 717) as template. Gradient PCR was performed using a dual block thermal cycler (Biorad, CA) for optimum amplification of promoter sequences. PCR-amplified fragments were gel-purified and cloned into intermediate pCR Blunt vectors using the Zero Blunt PCR cloning kit (Invitrogen, CA).

A variety of promoters were isolated and cloned in this manner, including the promoters for the L5L2 gene, (SEQ ID NO: 117) which encodes an L5-like ribosomal protein; an ubiquitin protein that is constitutively expressed in eukaryotic genes (Ubi2) (SEQ ID NO: 90); early response to dehydration 4 (ERD4) (SEQ ID NO: 137), a gene within a family that signals water deficiency and results in metabolic pathways to mitigate osmotic stress; methionine adenosyltransferase 2 (SAM2) (SEQ ID NO: 131), which synthesizes S-adenosylmethionine (SAM) from methionine and ATP; calmodulin protein (CalL2) (SEQ ID NO:103), which induces responses to change in Ca²⁺ concentrations in cells; and elongation factor protein (Elf1a) (SEQ ID NO: 94), which plays a role in translational elongation. A 2 kb PtPAL2 (SEQ ID NO: 158) promoter belonging to the class of phenyl alanine ammonia lyase gene family, which is involved in the biosynthesis of phenylpropanoids, was also isolated and cloned.

TABLE 2
Sequence-specific oligonucleotide primers for
amplifying various poplar promoters

| SEQ | Regulatory/ | | Sequence |
|---|---|---|---|
| ID | Gene | Primer | (listed in 5′ to |
| NO. | sequence | Name | 3′ direction) |
| 159 | GUS-NOS | ES190 | CGCGGATCCATGGTAGATCTGAGGGTAAA |
| | | | TTTC |
| 160 | GUS-NOS | ES191 | CGCGGATCCATGGTAGATCTGAGGGTAAA |
| | | | TTTC |
| 161 | PtUbi2 | ES778 | GAGGTCGACTACCTTCCCACCACAAGTAC |
| | | | TTCACCAGCA |
| 162 | PtUbi2 | ES779 | GAGGGATCCCGTTCTGTTTCTTCTTGTTG |
| | | | TTTCT |
| 163 | PtL5L2 | ES788 | GAGACTAGTCTCATCGCTTTGGAGAATC |
| | | | TTCCAGCAG |
| 164 | PtL5L2 | ES789 | GAGGTTAACTATGAAAGAGAGAAATTAGG |
| | | | GATTAGCCTTCTA |
| 165 | PtElf1a | ES843 | GCGGTCGACGGTGGTATAGGTAATTAAT |
| | | | AGATAAACAAGATG |
| 166 | PtElf1a | ES844 | GCGATCGATGATGAACTTTGGGAGAGAA |
| | | | ACTGGAATTGAT |
| 167 | PtERD4 | ES934 | GAGAGGCGCGCCCGAAATCTATCCAA |
| | | | CAAGGAC |
| 168 | PtERD4 | ES935 | GAGATTAATTAACATCATCATCATCAG |
| | | | GTAACGT |
| 169 | PtSAM2 | ES947 | CCATAAGCTTATTCTGACATCCAACG |
| | | | GCTATG |
| 170 | PtSAM2 | ES948 | CCATGTTAACTTTCTCAAGCTGCAG |
| | | | ATCACA |
| 171 | PtCal2 | ES950 | GAGAGGCGCGCCTATTCAGGTCAATT |
| | | | CATCTGACTG |
| 172 | PtCal2 | ES952 | GAGACCGCGGTTCTCTTTTGAAAAATG |
| | | | AAACCTAGAAGGAGAT |
| 173 | PtPAL2 | ES1055 | TTGGCGCGCCAGATTCTTCATAGAG |
| | | | GGAGCAAGA |
| 174 | PtPAL2 | ES1056 | CTAGTCTAGATTCTTATTTATATATAT |
| | | | GTTAGACCAAAGAAAAGCTT |
| 175 | PtDREP4 | ES1039 | GCTCTAGATCTATAATTAATGGAA |
| | | | TGTAATCCCTTA |
| 176 | PtDREP4 | ES1040 | CGCGGATCCATCTCTCTTGTCTC |
| | | | AAGTACTCAACAAAT |

### Example 2

### Expression in Particle-Bombarded Leaves Directed by Poplar Regulatory Sequences

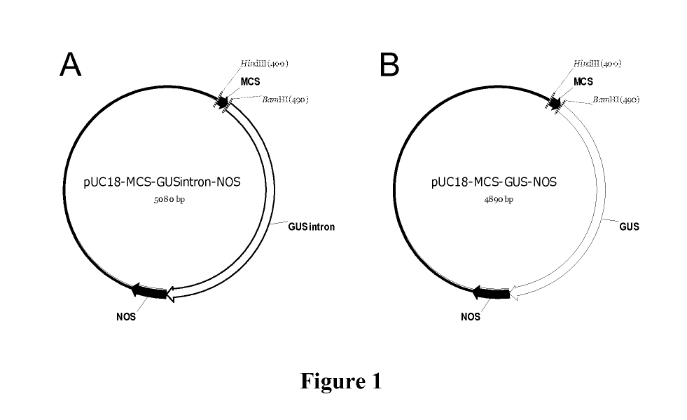

Construction of base expression vectors to generate reporter gene fusions pUC18 (Invitrogen, CA), a high-copy number cloning vector pUC18, was used for creating base expression vectors. First, a region comprising the coding sequences of the β-glucuronidase (GUS) gene with (GUSintron) or without (GUS) the catalase intron and the NOS terminator (NOS) was PCR-amplified from a plasmid. The catalase intron within the GUS gene is spliced out during the process of transcription in plant cells and eliminates background expression in bacteria. Restriction enzyme recognition sites BamHI-KpnI were engineered into PCR primers ES190 and ES191 (see Table 2). PCR amplified GUSintron-NOS and GUS-NOS fragments were digested with BamHI-KpnI enzymes and cloned into pUC18 vector to create the pUC18-GUSintron-NOS and pUC18-GUS-NOS vectors. Next, a multi cloning site (MCS) cassette comprising HindIII-AscI-PstI-SalI-PacI-NotI-XhoI-SpeI-HpaI-XbaI-BamHI restriction enzyme recognition sites was PCR amplified, digested with HindIII-BamHI enzymes, and cloned into pUC18-GUSintron-NOS and pUC18-GUS-NOS to create pUC18-MCS-GUSintron-NOS (FIG. 1A) and pUC18-MCS-GUS-NOS (FIG. 1B) constructs, respectively.

Cloning of Poplar Promoters into Expression Vectors

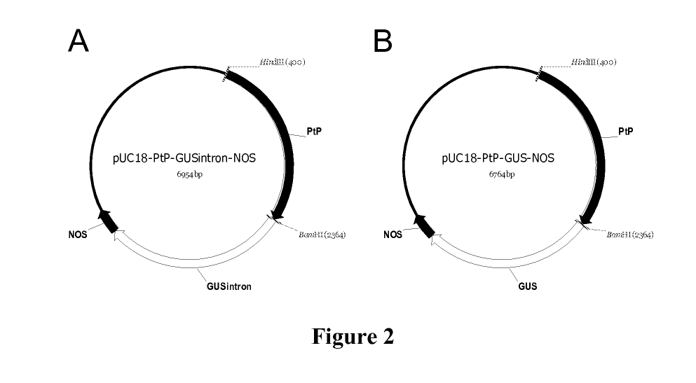

In general, poplar promoters isolated as described in Example 1 were classified into two categories depending upon the presence or absence of the first intron located within the promoter region. Poplar promoters (PtP) without the first intron were digested from the vectors into which they had been cloned in Example 1 with appropriate restriction enzyme(s) and then cloned into the pUC18-MCS-GUSintron-NOS vector. Poplar promoters with the first intron were cloned into the pUC18-MCS-GUS-NOS vector to create pUC18-PtP-GUSintron-NOS (FIG. 2A) and pUC18-PtP-GUS-NOS (FIG. 2B) vectors.

Particle Bombardment of P. tremula×P. alba Leaves

Preparation of Tungsten Particles

M10 Tungsten particles (Sylvania, Mass.) were used for microprojectile bombardment experiments. Stock solution was prepared by washing 50 mg of tungsten particles in 500 μl 95% ethanol followed by washing them in water 4-6 times. Finally the particles were suspended in 500 μl ddH₂O, Stock solution was used for a maximum of 12 hours after preparation. Twenty-five μl of the resuspended tungsten particles were mixed with 5 μl of DNA (200 to 500 ng/μl) in a microcentrifuge tube and vortexed for a few seconds. The mixture was allowed to sit at room temperature (RT) for 1 minute. DNA was precipitated by adding 25 μl of 2.5 M CaCl₂ and 10 μl of 100 mM Spermidine and leaving the mixture on ice for 4 minutes; precipitated DNA adhered to the tungsten particles. Fifty microliters of the supernatant was discarded and the remaining coated particles were kept on ice. Two microliters of the tungsten particle preparation was used per shot within 15 minutes. The mixture was discarded after 15 minutes and freshly coated particles were prepared as needed for subsequent rounds of particle bombardment.

Particle Bombardment

Young and fully developed leaves from 2 to 3 month old poplar plants were used in the experiments. The leaves were placed in a petri dish with wet filter paper and bombarded with coated particles at 60 psi pressure and 28 psi in HG vacuum. After particle bombardment, leaf tissue samples were kept in Petri plates under moist conditions for a 24 hr period.

### Analysis of Histochemical GUS Expression in Plant Tissue

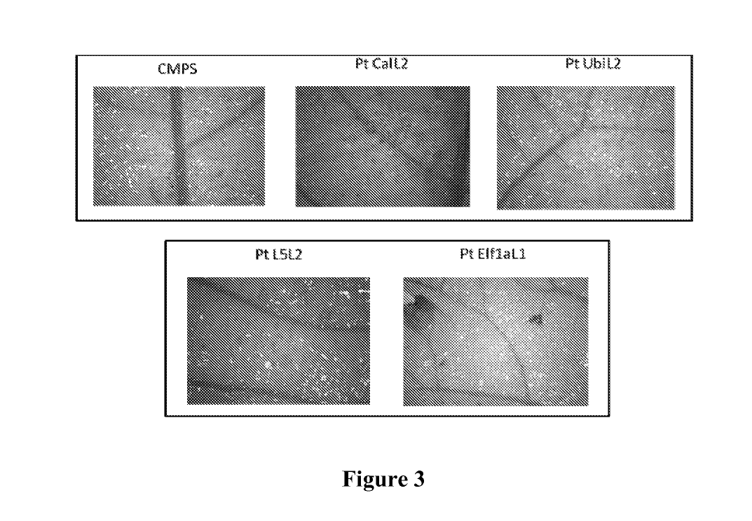

In order to monitor the GUS expression pattern for each transformed plasmid, bombarded poplar leaves were incubated with 5-bromo-4-chloro-3-indolyl glucuronide (X-Gluc) in a standard procedure for 24-48 hr as previously described (Jefferson et al. 1987, “GUS fusions: beta-glucuronidase as a sensitive and versatile gene fusion marker in higher plants,” EMBO J. 6:3901-3907, the entire contents of which is herein incorporated by reference). Tissue samples were cleared using 70% ethanol repeatedly until most of the chlorophyll was removed. Samples were observed for GUS expression (seen as blue spots) and images were taken using a Leica stereo microscope (Leica, N.J.). Results are presented in FIG. 3.

Based on the strength of GUS expression as evaluated histochemically, poplar promoters were classified into high expressers (PtUbi2 and PtCal L2 (SEQ ID NOs: 90 and 103 respectively); medium expressers (PtL5L2 (SEQ ID NO: 117)) and weak expressers (PtElf1a (SEQ ID NO:94)). A well-known dicot promoter, CMPS (Cestrum yellow leaf curling virus promoter; U.S. Pat. No. 7,166,770) was included as a control. These results suggest that poplar promoters PtUbi2 and PtCal L2 can drive high levels of heterologous gene expression in dicots.

### Example 3

### Poplar Promoter-Directed Tissue-Preferred Expression

In the present Example, poplar gene regulatory elements of the present invention demonstrated an ability to drive expression in tissue-preferred manner. Experiments described in this Example also demonstrate that inventive gene regulatory elements can drive expression in tissues other than leaves.

Stem sections were used for particle bombardments. Both transverse and longitudinal sections were used from 3 month old plants. Particle bombardment and GUS activity experiments were performed as described in Example 2.

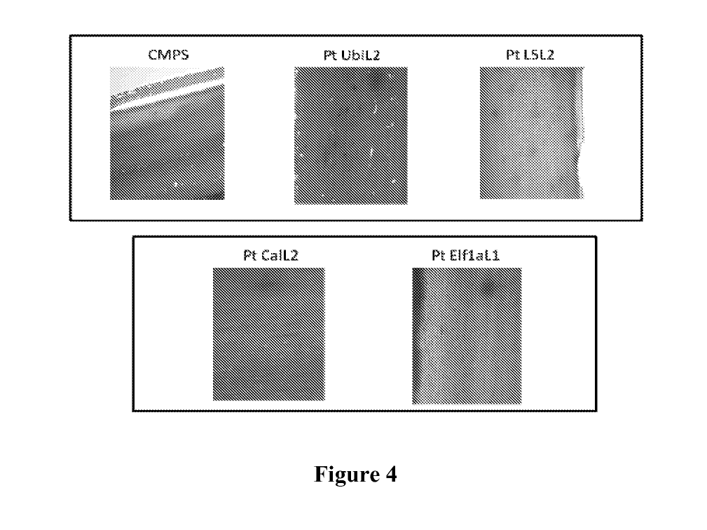

Results are presented in FIG. 4. In this transient expression system using GUS as a reporter gene, PtUbi2 (SEQ ID NO: 90) drove strong expression in both stems and leaves, while PtCal2 (SEQ ID NO: 103) drove strong expression in leaves and drove weak expression in stems. PtL5L2 (SEQ ID NO: 117) drove medium expression in both leaves and stems.

These results demonstrate that the suite of inventive regulatory elements provided by the present invention include regulatory elements that can direct diverse tissue-preferred expression patterns.

### Example 4

### Stable Expression of Transgenes in Poplar Directed by Inventive Regulatory Elements

In the present Example, inventive regulatory elements are used to drive expression of structural genes that encode proteins or polypeptides. Genes of interest (GOI) may include, but are not limited to, genes that encode cell wall modifying enzymes and genes that confer agronomically important traits, as described in the claims.

Stable Poplar Transformation with Poplar Promoters Driving Genes of Interest (GOIs)

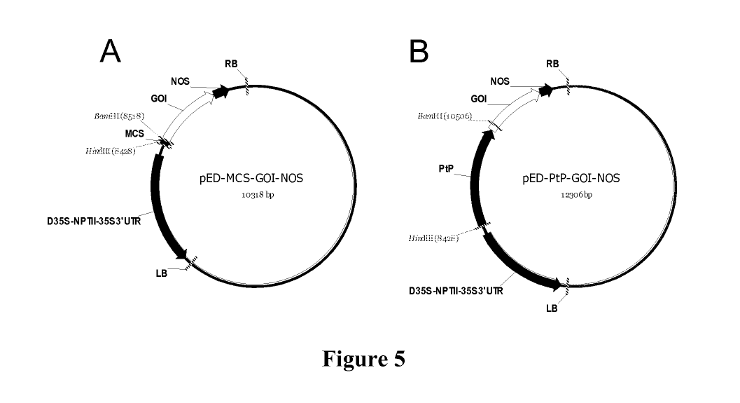

A plant transformation binary vector pED-MCS-GOI-NOS was created to allow cloning of different poplar regulatory elements (including promoters) to drive genes of interest (FIG. 5A). This vector uses kanamycin selection as the selectable marker for identifying and isolating transgenic plant cells. Plant transformation vectors containing genes encoding endoglucanase, β-glucan glucohydrolase, and GUS under the control of poplar promoters were constructed (FIG. 5B). Control vectors with 35S promoter and CMPS promoter were used to compare expression levels of poplar promoters. Poplar transformation was performed as previously described (Leple et al. (1992) “Transgenic poplars: expression of chimeric genes using four different constructs,” Plant Cell Rep. 11:137-141, the entire contents of which are herein incorporated by reference).

### β-Glucan Glucohydrolase Enzyme Assay

Total plant protein extract was made from fresh leaf tissue as described below. Leaf tissue was frozen in liquid nitrogen and homogenized in a bead beater (MINI BEADBEATER™, Biospec products) with 3 to 4 zirconium beads (2.0 mm size, Biospec products). Leaf protein was extracted by suspending the homogenized tissue in protein extraction buffer (50 mM MES, pH 5.6, 2 mM DTT, 1 mM EDTA, 1× protease inhibitor cocktail (Sigma P9599), 0.1% (w/v) Triton X-100). Samples were centrifuged and supernatants were used to determine total protein concentration using a Bradford protein assay. Two microliters of the total protein sample was incubated for 10 minutes with 200 μL of Bradford reagent (Quick Start Bradford 1× Dye Reagent, Bio-Rad, CA). The dye in the reagent turns blue when bound to the protein, which is measured at the 590 nm absorbance. Protein concentrations were determined by comparing absorbance readings to a standard curve of known bovine serum albumin (BSA) concentrations.

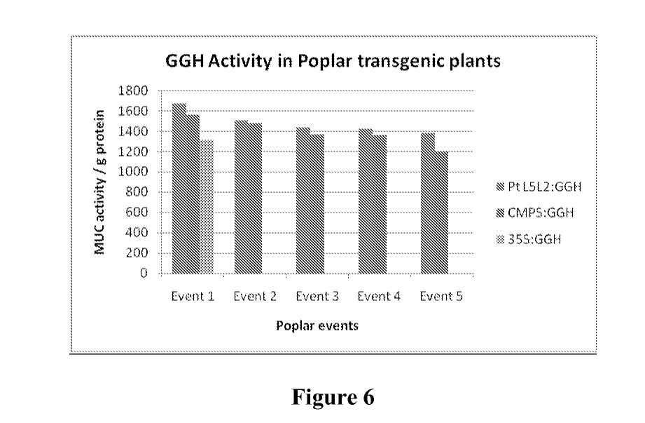

Glucan glucohydrolase (GGH) enzyme activity was determined using methyl-umbelliferyl-cellobioside (MUC) as a substrate. Fifteen microliters of protein extract was incubated for 1 hr at 85° C. in 200 μL of buffer (0.05 M sodium acetate, pH 5.0 and 0.1 M calcium chloride) containing 10 mM MUC fluorescent substrate. GGH enzyme activity cleaves the MUC substrate and produces fluorescent MU product, which is then read in a plate reader (Tecan GENios, MTX lab systems Inc, VA) at 355 nm excitation and 450 nm emission wavelengths. GGH enzyme activity was measured as strength of fluorescent signal normalized to mass in grams of protein used for analysis. The promoter strengths of the CMPS and PtL5L2 promoters were compared by measuring the level of GGH activity in protein extracts prepared from 5 different transgenic events. Based on the level of GGH expression, we concluded that the PtL5L2 promoter had similar or greater activity than the CMPS promoter (FIG. 6). In one event where the strength of the PtL5L2 promoter was compared with that of the 35S promoter, the PtL5L2 promoter was significantly stronger than the 35S promoter (FIG. 6).

### GUS Expression in Stable Transgenic Poplar Plants

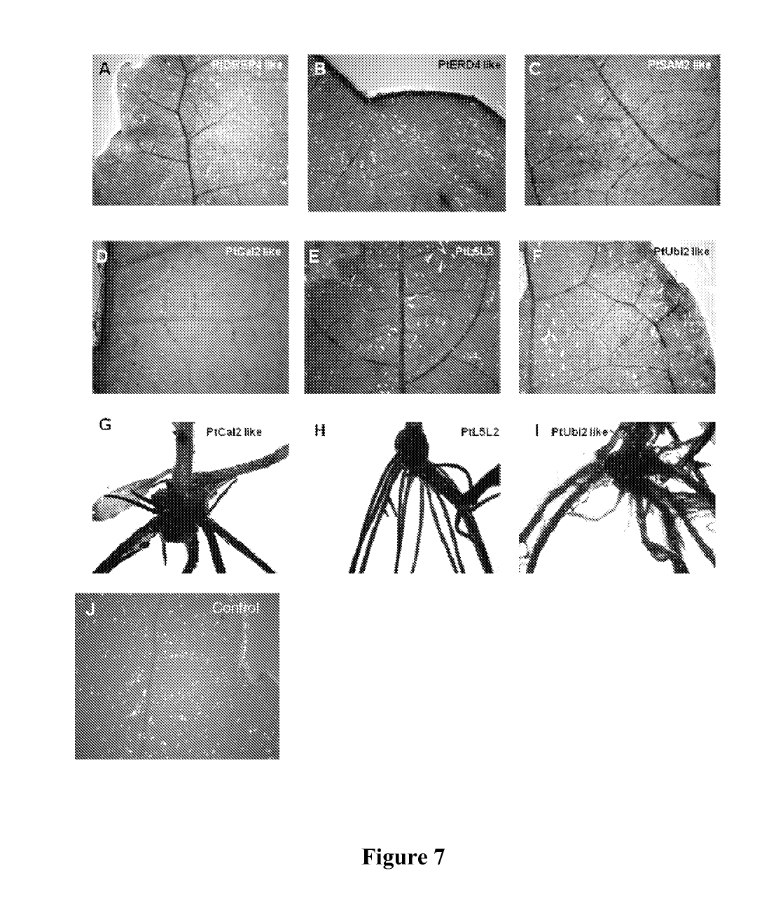

Trangenic poplar plants were transformed with constructs containing poplar promoters driving GUS gene expression. These promoters included PtDREP4, PtERD4, PtSAM2, PtCal2, PtL5L2 and PtUbi2. Transgenic poplar leaf tissues were harvested for GUS protein expression from one month old plants, while roots were sampled from transgenic seedlings grown in magenta boxes. Tissues were incubated with 5-bromo-4-chloro-3-indolyl glucuronide (X-Gluc) in a standard procedure for 24-48 hr as previously described (Jefferson et al. 1987). Tissue samples were cleared using 70% ethanol repeatedly until most of the chlorophyll was removed. Samples were observed for GUS activity (as indicated by blue color), and images were taken using a Leica stereo microscope (Leica, N.J.). Results are presented in FIG. 7. FIGS. 7A-F illustrate GUS expression in fully expanded leaves, and FIGS. 7G-I illustrate GUS expression in roots. The various promoters tested have differential expression patterns in leaf tissues. For example, PtERD4 drives strong expression, while PtCal2 drives weak expression, in the leaves of sampled plants. All transgenic tissue examined clearly showed significant activity compared to wild type poplar leaves (compare to FIG. 7J). Expression in roots appears strong in the three promoters tested.

### Example 5

### Poplar Promoters Drive Expression in Other Dicotyledonous Plants

The present Example demonstrates that regulatory elements of the present invention can also be used to drive gene expression in other dicot plants.

### Expression Constructs

Promoters PtUbi2 (SEQ ID NO: 90), PtL5L2 (SEQ ID NO: 117), PtPAL2 (SEQ ID NO: 158) and 35S were cloned into expression vectors to drive expression of an E1 gene encoding an endoglucanase. PtERD4 (SEQ ID NO:137), PtSAM2 (SEQ ID NOL 131) and PtUbi2 (SEQ ID NO: 90) were cloned into binary vectors to drive GUS gene expression (FIG. 5B).

In Vitro Enzyme Activity in Nicotiana benthamiana Infiltrations

Nine-week old Nicotiana benthamiana plants were infiltrated with suspensions of Agrobacteria containing the construct of interest. Each suspension was adjusted to an optical density (OD₆₀₀) of 0.1 to ensure that equivalent concentrations of bacteria were injected into each leaf sample. Young leaves were injected with 0.75-1 mL of bacteria suspended in infiltration media. The infiltration media was buffered to pH 5.5 and contains a source of sugar and phosphate for the bacteria as well as acetylsyringone, which aids the bacteria in infection of plant cells. Following infiltration, plants were grown for three days at 74° F. in a room with a 14:10 photoperiod, supplemented with 50 μmol m⁻¹ s⁻² florescent and incandescent light.

The infiltrated region of each leaf was cut out and ground in buffer. Total leaf proteins were extracted through an ammonium sulfate precipitation before washing and resuspension in buffer. Total protein concentration was obtained with a bicinchoninic acid (BCA) protein assay and compared to a standard curve of known bovine serum albumin (BSA) concentrations by a colormetric reaction (Fisher, Rockford, Ill.).

Enzyme Assays

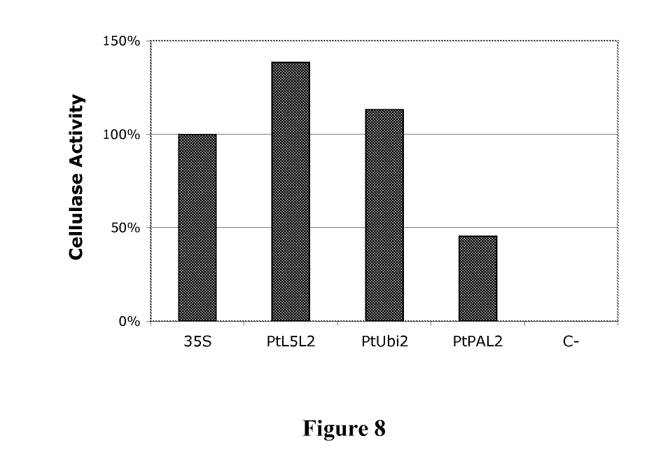

Total protein extracts were used to measure enzyme activity on methyl-umbelliferyl-cellobioside (MUC) and carboxymethyl cellulose (CMC) substrates. For MUC assays, proteins were incubated with the florescent substrate in buffer for 30 minutes. Endoglucanases cleave the fluorescent substrate; thus, the plate reader will read a stronger fluorescent signal from samples with higher endoglucanase activity. As shown in FIG. 8, all poplar promoters tested in this Example have detectable expression levels in tobacco leaves. The PtL5L2 promoter directs significantly higher levels of enzyme expression (as judged by enzyme activity) than does the 35S promoter, while the PtUbi2 directs slightly higher levels of enzyme expression as compared to the 35S promoter. PtPAL2 drove lower levels of expression of E1 in tobacco leaves, but still much greater levels than the negative control (leaves not expressing E1).

The polysaccharide CMC is also hydrolysable by endoglucanases. Plant protein extracts were prepared from leaves infiltrated with either PtUbi2:E1 or 35S:E1, then were incubated with the CMC for 1 day at 65° C. before being measured by a colorimetric dinitrosalicylic acid (DNS) assay to quantify glucose concentration. If a promoter drives high expression of E1, then a high glucose concentration is expected in the corresponding protein extract. After 24-hr incubation of endoclucanase-containing constructs on CMC substrates, glucose equivalents were ˜0.217 mg for the PtUbi2 promoter and ˜0.211 mg for the 35S promoter, consistent with the MUC data in FIG. 8.

### In Vitro GUS Assays

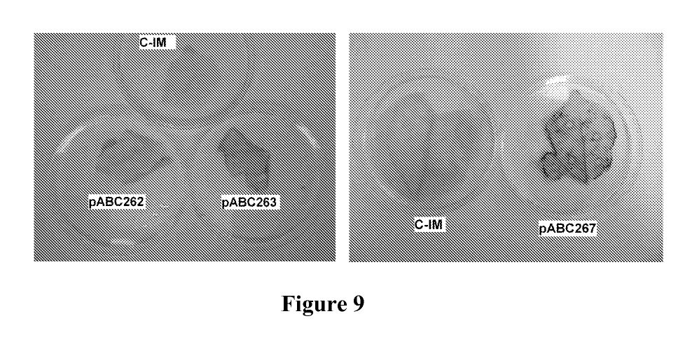

Similarly to constructs used in in vitro enzyme assays, constructs with promoters of interest driving GUS were infiltrated into N. benthamiana and stained for GUS activity as described in Example 4. The PtERDL4 (pABC262) and PtSAML2 (pABC263) promoters drove low constitutive expression throughout the infiltrated leaf area (FIG. 9A). In contrast, the PtUbi2 (pABC267) promoter drove strong GUS expression in patchy regions of the infiltrated area, concentrating most at the infiltration front and directly around the injection sites (FIG. 9B). Control plants infiltrated with infiltration media (C-IM) had no GUS expression in either experiment. None of the infiltrated samples exhibited GUS expression along the tissue surrounding the veins, suggesting leaf-specific expression in infiltrated experiments.

TABLE 3
Sequences of novel gene regulatory elements

Sequence ID: 1
Sequence Length: 2450
Sequence Type: DNA
Organism: Poplar sp.
AATTGTGTTTTCTGTTAGGTTTCCACTAACAAGTTTGTTCTTAGCCTAGGAGGATCTTGGTGAACCCATG
AAAACCAGGGGGGCCCTGTTTAAGAATGTACTCTTGTGGTGAATTATGAAGATGATGGGAAAACCAACGA
AGAGCTTTTTATCCCGTCGAAAATGTTAGAACCCGGTTATTACCCGGCCGGGTTGATTACATTGAGTAAA
ATCTAGTTCATCTAAAAATAGTGACCGAGTAATGTCAATTATATTGAGTAATTAAAGATATTTATTGACT
TATATCTTGTATGATAATTTTCAGGTGGTTCCAATAAATTGTATTACAATTCAAGGATGATTACATGTTC
TAAATCATTTTTAGCAGGAATTCTAAAGCATTCATTAGTGAGATTAAAAGGTAATTAGTGACCATCTAAC
TTGAAACCTTGCTCGAGCCGAGCCAAGACGAGTTAATAAAAAAACTTGATTTTTTTAAAGAATAATATCT
TTTTAATTTTTTAAAAAATTAAATTAATAAACCATGCTTTTAAGTTTTAACAGAGTCCACCATTATTGTA
GCTAAAAAAGAGGAGGGATATATTATTGCCCTCTTAAAAAAGGAAAGAAGGAAGAGGAAAGAAAGGAAAG
CAGCCAGCATTTTTATCCTCTCCTGTTCTAATTAAAGAAAATACAACCACAATTAGCGGCTGATTAATTA
AAAAGTGGTCAACCATAAATCTGTCCCACGTTGTGTTTGTTATGCATGGCACATCATATGCCTCATTTAG
CAAGATACAATGTCAGCCGTTTGATCCTTGATTCATCCATCTAGTCAACGACGGCTCGGTTGCTAGGAAG
CGAAAATCAAGGGTGTTATTGACAGGTAGTGGTGGGCATTACAACACCTGAAAAGACACACCGACATGAT
CTCCAGACAGTTGGAAAAGCAATGCTGAGTTGGCAATTAAAGTTCCTTTTTATTTTTTTTATTTTTTCCC
TTTTATTTTCCTATGAAATAATAACGAAAAAGAGGTCTGCTTGATTATAAAATTTTGCTTTTTTTAGCGG
GGTCCTTTTTCAAGGACTAATGTACCCCTCGACGCTGAAATGCCACAACCATGCCTTCATTTTTGTCAAT
TTTATAATTGAGAAGGGTAATTATGGAATTCCAAGTACACTAATCACGATTTCCTGTTTAACAGGGTTTC
TAATCTAAACTTACCACCACTAGCAAGGTAACTGTCCTGTGAAAAAAAGCAAGGCAAACACGGTCAAAGA
CAATGCAATGAATTTAGGATGGGAATGCCCTAATTTTTTTTTAGCTAAATTTCATTTTTTTACCTTTTTA
TTCCCACTTGTTTAGAAAAATACTTCAGAATATCTCTAAAATAGAATAAAAAATAAACAAAATATTAGGG
TTTAGTTACATTGATAAGTTTTGTAATTGAAAGTATTAGTATTTTAATTTTTTAAAATGAAAAAAATTAT
ACCTTAGCATATGAAACTAAGATAAAAATTGATATTTTTTAATATTATTTAAAACACAAAAGCTAAATAA
ATATTTTAATCATATATGGATGAATCTTTAATATTATAATACATTATTTTTTAGATTAGCGGATCAAAAC
GATTCAAAATATTAAAAAAATTAATTTTTAATAAAAATTTTTTGAATTTTTATAAAATATAATTTATACC
GCGTTGTGATAAGCAATGAAATGGTCAAATTGAGATTTGAGAGATTGGTCAATAACCTTAACAAAAATCC
CACAATTCAAGGGCTTGCCTGACCAACATGCCCTTGGCCAAGGACGAGAGGTCAAATGAGGCATTCTAAC
AAATATCAAGGGCATATCCGTCCACACAAATAAAACAACCGAAAGCGAGCCAGCTGTGTACACAATGAGC
ATGACGCGTCATGGGGTCCGTAGATCAGATGCTCGAAACCAACGGCCAAGATCATCCCAAAGCAAATTTG
AAGTTCATTTAAGTTGAATTTCATCCGAGGCTGAAATTTCATTTCACATCTTCCCCTTTTTTAAAACAAG
AGGGCGGGTACCTTGAACAAACGCTCACACACAGCGAAGGAAGAAAGAAACACAAAGAACAATTCAGTTT
TCTGAACGGTATGCTGTTTGAATTTCAAAATTCTTCATCTTTCTGATCCGGGTTTAGCTCAGATTATAAC
ATCATTGTGTCAAACCATTAAAAAATTTGATCTTTAAATGTGGGTTAATTTTATTTTGTGATTTAGCATT
GCTAAAGTTTTGATCTTTGATGTGATTTTACAAAATGAACATTCAGATTTTGATTATGATTTGTAAAAAG
GAAATGGGATCTTTTTATTTTACTTTCCATTGGGATCATCATGCTCTTTTTGTCAAAATCGTGCATTAAA
TTGTCGTGATTCCCTATTTGTATAGCATTTTAATGTAAGGTGGCATTTTGAACGTGGCAGGTGAGAGGAA
Sequence ID: 2
Sequence Length: 3003
Sequence Type: DNA
Organism: Poplar sp.
TTCGGTGCTTGTCTAGTAGAAAGTGGAGTTCACGTTCCCATTCAATTGCCTGAGGATCGTAGGCTTTTAT
TCTAGACAGCTTAAATTAATGGAAACTTAGCAACGAGGATAGTTCGTCGTCAAACCTTTATTTCTTTTCT
TGTCTTGAATTATTTGGAATAGTTTTTTGAATCGTATTGGTTTAGCTAATTTGTTGAAATGGTGGCTTCG
TTACGATGTTTGGTACTTTTTTATTTCTTTTATTTCCATTCGCTTGCTGTTGATGCCAATTAAGGCCATG
TAGCAGTTGTTTACTGAGTGGTGGGTGAAATTCATTATCCATGTCACATCAGAACTTATTCTGAAGCAAC
ACAGGCTACTAGCTAACCTACTTGCAGACTGAAAAAAAGGATTTGGATCATAACACAACCCCCTCTTAGT
AAAATAAAACTCACATTTATGATCCATAAAAGAAGTCTGCTCACAACAGGCAACGTGTTAATAGCCTAAT
TCATCTAGCAGATGTCTCATGCTGCTGCATCCATCCATGAGAAAACCCACAAATTGCAGCGCCAAAACTA
CATCCACGTGCAAGCTCGTTCAATCCCTTGACATTGTATTTGGTGTTTATCTAAAGCTTAGAGGCTGGTG
GAATCGATCATTGATGCTTTGCATACGTGGAACATTGGTGACCAAATCATCATTGCCAATGGTTGCATTC
TGCTGCTTATACGGTAACCACTTTACGGGGAGGGAAAAAAAACAGGCGTTTACACCACAGAAAATCGAAG
GTTTCAGACTTTTTGCTCCAAAACGAGCCCACCCTTGTGATTCCAATGCTAGCATTGTCCAAGTAAAAGA
GAAAGGGTCCGGGCCATGGAAGCGCGCTCCAACCGTTATGTGCTGGAAAACGTAGCTCCACCTAGTTTAG
AATGGTCATTTTAGCACAAAAGGCAGACGCTAGACTGCATCATTTATCCAACAAAATCTTTGATTCTTCC
ACACCTGGCCATTTCATAATCAAAGGAATGTTCCTACCCTTTTTACTCATGAAGGGAAGAGTTGTCGCAG
CGATGCACAGGTGCAGCTCACTCAAATACTCAGATAGTGGGCTGTAGCCTGTACTAATGGTATTTAAAAA
TAAAATAGAAACTTGAAGAAGAGTAGCTCGATTAGCACTAATCCATCATCTCCTTCAAAACCATTTTTTT
CATATTATTAAACACATCTTCTTAAGACTAACGTAAATACTTACATGCATGCATGTGTATATATATATAT
ACACACAGTGGTTGATACTTGATACCAAGTTTATGTTCTAGAACAGTTGTGTGCTCTAAATAGTATGGTA
GATTTTTTAATTAATTTTTGTAATAAAATTATACTTATCCACGAAAATTGAGCTACTCTCGTTGTTAAAT
TCTAGTTTTTTCATTGAAGAGGTAAAAAAGAGATATCCTGACAGCATATTTATCACCGTAAGAAATTTAA
TAACTTATACAAAAAAAGGAAAAAAACAATAATAGTAATTTTTATGATTTGTTTAGAGTATGGTAGAGGT
TATTTTTAAAGTGTTATTTATTTAAAAATATATTAAAATTATTTTTTTATTTTTTAAAAATTATTTTTTA
TATTTATACATTAAAACAATAAAAAAAACATAAAAAAATTAGTTTAAAACTAAAAAAAATCAAAACATTT
CAAAAATACACCATGCCAAGCAAGAACACTACGTACAGGTAAGATTTGTAAGAAGGGAAAAAAGAGGAGG
GGTAAAATAGAGAAGACAGGAAGGAGAAGAAAAAGGAAATGACTGGAAAACCGGAAGGAGCTGGCTGGAT
CCAGCAAGAAATAATAACCCCAGATAGATAGGAAATATGAAAAAAGAAGAGCGCATGACGCGTTCCTCTC
TGTAAGTTGTGCCCACAAAATAAAAATAAAAGAATCCAACGGCCCACAACTCATCTCGCGTTCCATATAT
TGAGCTTCTTCCTTTCTCTTTCCAAAAGCTCAAAAAAAAAAAAAAAACATTTTAGTTTTGTTTGTGTGCG
TCGTCGCCTCGCTCACTCTCTCAAAATCCCTCCTATAACAAGGTCGGTCCTTTTCTTTTTCAATCAATCG
CTCTTCTATCTTTTCGATCTGCTGTATAGCGGTCTGGATCTGATCTCCTTTTGATTCTGCATTTTGCTTT
TTAGTATTTTGTAATGTGCGATTCATGCTCGCAAATCATTTTTTTTATCTGCATTTTGGATTCTTCTTAA
TCCCATATTTGGATCTGGTTTTATTATTATTCCCGGGTTCGTTTTGTTTAAAATCTGAAGGAAAATTGGG
AAATTAACAGTGAAGACCGTAAATTTTACTGTAATTACAAAGTTGGAAATGGATTCTAAATGCTCACTTT
TCTTTATGTCTGTGTCTTTAGATTTCGTTTGCAATTGCTTTAAAAATGTGATTTAACATTATTCTGAAAT
GATATCCGAGATTTACTTGCCTATTCTCTTAAGTCTTTTTTATGCATTTTGATTTTGATGCGATATATTT
GTTCACTTTATTTTTCCTAATCGATTTCTTTTCCGTTTTTAAGACACGGAAACACGACGGTTGGTTTGCC
ATTGCATTTTCTTTTTTTGGGGGCCAATGTACACGACGGTTGGTTTGCCATTGCATTTTCTTTTTTTGGG
GGCCAATGTACACGACGGTTGGTTTGCCATTACATTTTCTTTTTTTGGGGGCCAATGTTCAGTAGTTAGA
TTTCCAGATTAAGGTGTTTGTGTAATAAGACATGGGATCTATAGATGTGATACATTGATTTGCAATGTTG
CTGTTTGTTTTTTAACTGATGAAGGCGAAAAAACTCATTTGCTTGTTTGTGTTCTTTTTTTTTTCAATAT
TTTATTTTTTACTCTGTTTTGCATTAATGCATGGCTAGTTAGGAGATGATTTTGATGTTTTTGCTTCGCT
TGATAAAAGAAATTTATTCCTTTTTTGTTGATTATCACTGTTTGGTATTACAGGCAGTAGAAG
Sequence ID: 3
Sequence Length: 2000
Sequence Type: DNA
Organism: Poplar sp.
TACGTTGACCACTGGTTTTTGGTTTCTAGATCTATAACCAAATCATCATTGCTACTGGTGACATTCTAAT
GCCTATACAGTATGCACCTTGCTGGGAGGGAATATAAGGTGTTTACACTCAAGAGATTCAGAATTTCCAG
CTCTTTTGTCCCAAAACGCACCCACCCTTGTGATTCATATGCTTCGACTGGCAGCATTGTCCATGTAAAA
GAGAAAGAGTCCGTCCGAGCCATTGAAGCGCACTCCAACTATTATGTGCTGGAAAAGGTAGCTCCAGCTA
GCTTGGAATGACCATTTTAGCAAAAAGTAAACGCTGGACTATATTATTTATCCAACACAAATCTTTGATT
CTTCCATGAATCATGGCTTGCCATTTCATAATCAAGGGAAATTTCCAACCTTTTTACTCGTGAAGGGAAA
AGTAGTAGTAGAAATGCACATGTCCAACTCTAACATGCTCAAATAATGGGGACTGTACTGACAGGATTTA
AAAAAACTTAAAAAAGGGTAAATGGATGAGCACTAATCAATCATCTCCTTTGTAATATTGAAAGAGAGAT
CTTTGCAGCACGATATCAGTACTAAACAATTCGATTATAGTTGACGTGGCTCAGACCCCGACAAAATTTA
GGTCAATTTGCCTGTCACTATCGATCTTACAATAAAATCCCCATATAGAAATAAAATTAAGGTAGTGTAC
TCCCGGTAAGAAATAAAAAAAGAAAAAGAAAAAGAAAAAAGAGGAGGCTAAAATAAAGAAAACAGACGGA
GGAGCAACAAAAAGAAAATGACTGGAAAACCGGAAGGAGCTGGATCGAGCAAGAAATAATAACTCCAGAC
AGACAGGAATAGGGAAGATTTAAAAAAAAAGAAAAAAGAGCGCATGACGCGTTCCCCTCTGTAAGTTGTG
CCCACAAAATAAAAATAAAAGAATCCAGCGGCTCACAACGCATGTCGCTTTGCATATATTTTGAGCTTCT
TCCCTTCTCATTCAAAAAGAGAAGAAGGCTAGGCAACCCACCTCGAAAACATTTTATTTTGTTTGTGTGC
TGGGCGTCGTCTAGCTTTCTCTAACTCTCTCAAAATCCTTCCTCTCCTTTAACAAGGTCCATTTCTTCCT
CCTCTTTTCGATCAATCGCTGTTCTCTCTTCTCGATCTATTATTTAGCAGCCTCGATCTGTGCTGTTTTT
TATTTTGCATTTTGCTTCATAAAGCTGGGGATCTTGTAATGTGCTGCGGCTCATGCTGAGAAATCATTTC
TTTTTGTTCCTTTAATTTTGATCGTCTGTTTGGATCTGATTTTGTGATCATTTTACTTTTTTTGTCTAAA
ATCTGAAAGAAAATTGGGAAATTAGCGACGAAGAACGTAAAACACATTAGAATCATAATGTTGGAAATGG
AGTTGTTGCGTTCGCGTTTTTTAGTGGATGTCTATGTTTTAATTTAGCATGCAATTGGTATGTTGCGTTA
AAGAAGAACGTAATTTCGCGAGATTTGCCTGTTCTCTTTAATCATTTATTTGCAGGTATTCACAAATGCT
TCGATGCATTTTGATTTGGATGCGATTTATCTGTCCATTTTCTGATTATTTTTTAATTCCAAATATTTTG
TTTTTACTAGTTTTTAAAAAGACAGAAACACGATGGTTGATTTGCTATTTCATCCTCGTTAATATTAAGT
ATTTAGTTTTGTGCAATGAGCTACGAATCTATAATGAATGTGGTGTGATTGATTTGTTTTGTGTTGCTTT
ACTTAATTTCAAAAAATATATATATATTATCCTCTGCTTCCTTTGTTGTGTTGCTTTACTTTACTTTATG
CTAGTTGCAAAATCTGGTTTGATTCTGTCTAATAATTTGAATAAACAGTGTCATGTACTTATTACGAGAT
GATTTTGATGATGTTCAAGAAGAGAAAATGAATTGATGAATCTGCTTTGGTTGATAAAAGAAGTCCGTAC
CTTTTTGTTGATTGCTGTTTGGTGTTGCAGGCATTAGAAG
Sequence ID: 4
Sequence Length: 3073
Sequence Type: DNA
Organism: Poplar sp.
AAATTGATTAGAGAAAATTATATCCCTTCTCATTTCAAAAGCACGGTGCACGGTGAAAAGACTAAAAATC
TTTAAAAGTAAACATTTCAATAGTAGCATTAAGTGATAATTTGGTATTTTCAAAATAGTTTTTTTCATTA
TTATATGTAAAATGAGAGACAATTTTGTGTTTGGATATATATAAAAAATCAAAAGTACAATGAAAACGAC
CAAAATGCCATTGGAAATTAAAATTGGCCTCAAGGGATTTTTTCAATTTTTTTCCAAAAGTTTTTTCAAT
ATTAACATGTTAGATCAGGGGTAAGTTGATATTTAACTAAATAAAAAACAAAAAGGGATAAGAGCATTTG
ATGCATTTAGAGGCACGCAGGTGACGCCCGCTACCTTTGAGCAACGCATGTGGCGCCTCCCGATGTTCAA
AAATACCCTTTCTGTTGTTGTAAGTGCTTATGTGTTCTTTTTTTATTGGTACAACATGTGTCTCAAATGG
TGGCCCAAGTTGTAACCGGTAGGCCTTTTTTCCTCTCTTCTTTTCTCTCTCCTCCCCCAAAAATTAGAAC
TTGGCTCTAGTGTTTGTTGGTATTTCAATTCTATTTCTTACTCTTTTAATTGTTAATTTTTTGTCTTGAT
TCTTTTGTAGAAGTCTTATTTGTTTTTAATTTCATCATTCAATTCAAATTGAAAATATTATATTTTTCAA
TTTAATCCTCATTATTTTGATTTCTAATTTTCTTGTTGGTTATTTTGTAAAAGTTATCATTTTTTTTTTA
ATTTCACCATCCAATCAAAATATTGTTTGTATTTTTTATGTCAATTTTGATCTTTATTCTTTTGAATTTT
CTTGGTCATTTTACTAAATTAGTTTTTCTTTTCAATTTTGATTCTCATTCTTTTTATCACTATTTTTTGA
ATCCCTTTGTATAATTAATTTTTTTTTTCAATTTCATCATTCAATACTTGATTGATTAAGAATTGAACTT
TATGATTTTTCTAAATTGAGTGTTTCAAGTCTAATAACTCGGATTACGGATTTGAAAAACTAACATGATT
TTTTTTCAAAAAAGAGCTTTATAATCCTTTTTATTTTTTATAGGGTTATTTCAATCTCATGATTTGTGCC
GCGAGTTTAGTGAGATATCCTGGTTTGGCTCGACCTTGATTACAATAGTTATATATTTGCTACACTAACT
CGGGTTGATTAAGAATAATTTATTTATGTCTTTTTTATCTATTTTTAGCACATAAACATTAATTTTTATA
AAAAAAAAATAGCGGGTACCGAAGCTGGTATCTCAGCTAAACAGTAAACCATATTCATGATAGAGAATTC
CCGTTCAAATCACAACCGACCCGACGCCACTTGACCCTACCACTTCAAAACAAACCAACCATCCCCAACC
GCCAAAGCCGTCCAAGTCAAGATCCGACGGCTGAAATTAAACAAAAAGAACAAACCGAGTCAGCAAAGCC
GAAAGAGTCCCAAGTCCAGAACATGAAACGGAGGTCATAAGAAAATAACAGAATCAGTTACAAATTAGTA
TCTCTATTCTTACAAAACAAATCAACTAATCCCTCTAATTTTTAATTAATTAATCTTTTGTCAATCATGC
TTAATTTAATTTTAAGTTATTCCAAAAGAATTCTTCTCATCAATATTTATATAATACTTATTTATTTATC
CAAATTTTATGTTTATTATAACAAAATAAATAAATAGATTAGAAAACAAATATAAAATGAATGAAAAGAA
CTAATTAACCCGTTTTTAAAATTATAAGGTCTATTTTATCTACTTTGAACAATTACAGGGACTAGTTTTT
ATAACACTGAAAATAAGAGAGACAGAGGGAAAGGCTGTCAGACAGGTCACCCTATCTTTTCTCGTGGTGG
AAATGATTCCTCTCTCTAAAAAATTTGGTCTTAATTTCATTTGCATTTCAAAAATTCCTTCTCTCTTTCT
CTCTCACTATATATATAAACCTCCTCTCTTCTCTTCAGCTAACCCTTACACTCCTCCATTTTCGTCTCAC
CCTCTCTCTCGAGCGCAACCACCAGCTAGCAAGTCAGGCAGGCCACATCTTTCGCTAGACTTGGCTTGGT
CTGTTTCGCTCTCTGTCTCTAGATATCTCCTCTGTCTCCGACTTCAAAAGGTAAGTGACTGATCTGTATT
TGTGTCGATTTGTTTTGAATTGTGTAGATCGGGTGAACGGCTCGTTTTCTGATTTGAAATTGGTTGAATC
GTGGATTTTTTCTTCGCTTTATCGGTTTTGTGTTACCGATCTAAGTTAAATTTTGGTGTATTGTGTTGTT
GAAGGTTAGATCTAGAGGTTTTTGGTGGTTTTGTTTGGATCGTTTGATTTGATGGCTTGATCTGGTTTTG
TTGTGTATAGATCTGCTGAGATGTTTTTAGATCTGTTGATTTGACCTTTCAGATTTTGATTTTGATTGCT
ATCTCGTATGAACTTGAAGAAACGTAGAATTTAAATCGTGTGCAAATTGGACATTTTATGTGGTGATTGA
TTGAGATTTAGCTTCAGCTGTGGTGTCTTGGATTCATTTTCAATGACATTTGTGTTGTTTTTAAGCTTTA
AACTCTTATTTTTTTTCATAAGATGGCTGGATTCTGTGAGTAAATGGTAAACATGCTGATTGGAATTTGA
ATCGTGTGAAATTGGAAATGATGTGTGGTGATTGCCTGAGATTCAGTTTAAGCTGCAGTGTCTTGAATTT
ATTTTTTTAATGATATTTGGGTTGTTTTCTAAGCTTAAAATTCTTATTTTTTCATAAGATAGCTGAATTC
CGAGAAGAGTAAAATTGTAGTGAATGCTGATTGGAATTTGATGAGTAAATTTTATTGTTTGCATTTGTTT
GAAGCTTTGGATGGTAAATGATTTGGAATGATTAATTGGCTGGTAGATTTCATTTTGTGAACAGGGGAGA
AATTGGATATGTTGTAGTTGATTATTCTTTATACAGATGGCTCTGTAAATTATCTAGAAAGATTTTTCTG
TATTTATGGTTGCTTCTTTTGCTCTTTTTTATTTTTTATTTTTTTGTACAGAATTTGTAGAAA
Sequence ID: 5
Sequence Length: 3818
Sequence Type: DNA
Organism: Poplar sp.
ACATTTTTTCCCTCTGATTTTGATTTTTCACTGTCTGCAATAATGACATCAGAGAAAATATTAAATGCCA
AAAAGAAATAAAAGATTCTCGAGTTTTTGACAAAAAAAAACCTGTAAGCCAAGCCTACCCAAGCCGACCC
CAGGCTTGCCATTTTTCAGACTCGACTCCTCGTGTCCCTGCTTAATAATAAATGTTAGGGCAAAAATTAC
ACACACACAATATTAAGATGATGTAGTTTTTTTTTTTTTAGTTTAACGTGGGTGTCCGGGCCAGCTTACG
TGCACCTCGACTAATCCCACGGGCCCTGAAGTTAACGACCATGTAAGCCTTCAGTGGCCATCATATGAGC
AACCACAGGGCTCGAACCTGAAACCACGGAGGGAGCAAACCTCTTGATCCCAAGTTCTTACCACTGGACC
ACCACCTAGATGGTTAGATGATGTAGTTTTTAATCATCTAGTAGGTGGTCCAATGGTAAGAGTTTGAGAT
TAAGAGATTTGCTCTCTCTGTAGTTTTAAGTTCGAGCTCTGTGGTTGCTAATATGATGACCACTAGAGAC
TTATTTGGTCGTTAACTTCAGGACCCGTAGGATTAGTTGAGGGGTGCACAAACTGACCCGGACATCCACA
TTAATAAAAAAGAAAAAAGAAAAAGTTGATTTAGTTTTACATTCAAATTATTTTATTTTTTCAAGATAAA
TTAACAGAGACCATTAATAATCTTACAATTAAGATTGATATAAGAAATTGTTTAATGGTGAAGTTTATAT
TACCTATATAACTGGAATATAATTTTGAGTTTTTCTCTGAATGCAATTACTCTGCTCCTCCATTTCAAAG
TTTCTATAATTATCATATTTTTTCTCAAGAAGGAAAGTTTCTAGTCTAATCAAGTGAAAGAATAATTCAC
CAAATAACTGAATAAGAACATGTATATTTTATAATAAGATCAAGAATATGAAATATGATTTTCTCCTATA
ATATAAATTAAATAAATTTATAGGGTTTTTTATATTCTAAAATATAAGTTTAATGAAAATATAATTGACA
GAAAAATAGACATGAAGCTTCTTCTTTTTTTCGAGTTTCTCTCTTATATTCTCTTAAAAATAATTGCTTT
AAAGAGATTATAAGTAATTTCTTACAGCATTTAAAGTGAATATTATTTTTTTATCATATAAATATTAGAA
TTTTTGACAAAATACTATCATTTTATGTTTTAACTAGGGGTGAGCAAAAAAACTAAAAAATCGATTAAAC
CGAGAAAACCGGAAAAAAAATAACAAAAAAAACCGAACAGTGAAAAAAACCCGATTAAACTGATTAAAAT
TTTGAAAAAACCAACTTGTTCGGTTCGGTTTTATAAGCATGAAATCAAAAAAACCGAACCAAACCCAAAC
CGGAAAAAAACTAAGCCAAACTGAAAAATCGAGCCAAATCAAAAACCGAAGCCAAACCGGTCTGAATTCC
TAGTTTTAACATTTTATGTTATGATATATTAGATCAGTTATATATTTTAATATGAAAATTCAGAATTTTG
TAAAAAAATACCATCAACTTTATTTGAGTTGAAATTGACTCGATTTTTTTTTTCTAAGCTTGAAGTGGCT
ACTTTATTGCCGTTTATTAGGGCTGCTAATGATGATTTGTACTATTAAGGGCTGCAAAGAGTTATTGAAA
CGTATATTTATAATAAATTATATTTAGATTTAGTTGGTGGTGGCTTGTAAAATATATTTATATTTAAAGA
ATTATAAGTCATAAAACCGTTCAACTTGGCAACATTTATTTTTATTTATTGAAAAGATGTAAAAATTGGA
CCGATTTAGGAAAGTGGGAAGAATACTTGATTTAATAAAAACAAATAAAATTTTATAAAGAAACAAATCT
TACATGTCTATTAATAGATGTTGGTTCATAAATATTTTGCATATTTTTTTTAATAAGAGGATGCCTTAAA
ACCAATTTCTTTAGGTAAATAATCTATATCAAGTTATTGCACACTAAAATAGAATATAAAACCCTATAAA
TAAATATAAATTTATTTACAAAATGATTGATAATGTAATTGGAACAAAAATCATGAAATGCATGTTTTTA
TAGACAATTTCTTATGCTTTTTTTTTTTTAACTCTATAGAATTATAATTACTAAACTTGGTAGGAGGTTT
TGATATTTACAGGATAGTTATGTTAACTCATGTTTATTATTTTTATATTTTTTAACTATTAGTTTTGACT
TGATTAAGTTGATATTAAATTTAATTATCTAAATTAATGTCAAAACCATTTTAATTTAAAATCCATTTTA
ATCATAATTTTTTAGATTAGTCTATTAAATCAAGACAAGTTTAACAACTATATATAAAACCATGAAAAAT
CAATGTACAACCAAATAGGTTAAAAGGAAAAAGAAATGATTTACAAAATGTTTATTATGTTTGTGTTGTG
ATACTAGTTGCTTTTTAAAATATTTTTTATTTAAAAATATATTAAAATAATAATATTTTTTTATTTTTTA
AAATTTATTTTTAATATTAGTATATCCAAATTATCTAAACATATAAAAATTTAATTTTAAAAAAATTAAA
ATTTATAAAAACACGCAGTGCGTAAACAAACACAACTTATATCTCAAATAACGGTCCCATCATACCCGAC
CCGACCATTCAAAACAAACCAACCATCCCAGCCGCCAAAGCCGTCCAAATAAAGATCCGACGGCTGAAAT
TATAAGGAAAAAAAAGTCAAAAACCAAGTCAGCAAAAGCCGAAAGAGTCCATAGCCCTCAAGGCCAGAAC
ATGTAGGGAACACAAGTGAAGTCATAAATAAAATAAAATAAAAAAAGGCTGTCAGCCAGCCAGGTCACCC
TATCTTTTCTCGTGGTGGAAATGATTCCCCTGCCTAAAAATTTAGTCTTAATTTCATTTGCATTTCAAAA
ATCCCTTCCTTTCTATCTGTCTCTCACTATATATAAAATCGTCGTCTCCTTTCCTCAGCTAACCCTTACT
CTCTCTCTCTCTTTCTCTCTCGCACCAGCAACCGCAATACAAACAAGCAATTTAGGCCAGGCCACGTCTA
TAGCTAGATTTGGCTTGGTTCGTTCTCTGATTATTCTCTCTTATACTTCTCCGATTAAGGTAAGTAAAGG
ATCCCTTTCTCTATCTCTTCGTCTTGGTTTGAGCTTTGTAGATCGTGTTTTGGTTAAACGCCGTCTTTTT
GTTGTTTTTGGTTTGGAATCGGTAAGTTTTAACGTTTTTTTGTTTCCGATCTATGATTTTTGTGCTGTCG
AAGGTTAGATCTAGAGGTATTTGGTGGTTTGATTTGGATCGTTTGATTTGAGAGCTTGATCTGATTGTGT
TGAATCCAGATCTGTATGTTTTTGTGACAAGTTATTAGATCTGTTTGATTTTGACCTACTTCGTTTGGTC
ATTGTTAGATTATAATCTGCCAGTGTGAATTAGAAGACATTGGTAAATTGGAAATGATGTGGTAATTGAT
TGAGATTTAGGTTGCATTTCTGTGTTTTAAGCTTAAAATAGATGATTTTCGATGGATATTTGGATTCTAT
TATAGTAGGAATGATAACGAAAGATAATCATGCTGATTGGATTAAGCTTTGGATGGTTAATGATTTGGAA
TAGTTAATTGGCTAGTCTATTTTGATTTTTAAATAGTGGAGGAACTAGATATGTTGTATTTGATTATATG
CTTCATAGATATGTAGATGTATTTGTAAATTATGTAGAATGTATTTTTCTGGTGTGAAGTTTGTTTCTTT
TACTCATTTATTGACTTTTTGCACAGAACTTGTAGAAA
Sequence ID: 6
Sequence Length: 3354
Sequence Type: DNA
Organism: Poplar sp.
AACAACTGGAAGTAAGACATGACAAGAGAGAGTGTCCAAAAGATTCCCATCCTCCAAAATTCTACTGTCA
AGATGAAACTCAAAGCCTTCTTCACTTCCCTCATTCTTCTTCTTCCTCTTACCTATGAACAGCAAAGCTT
GCTATATCCCCGACCACTGCTGACATGAACTCATGGAGTTCTTGATTTCATTAAAGGAGGCCTAGCCCAT
GGTAGAATTTTCAACAGGCCTAAACAGAGGTTCTGTCATACGAGCTGCCCAATGGAGAGAGTTCGAAACA
TGAGAAGAAAGCCCAAGTTAACCAAAAAAAAAAACTTGTATTTTTTTAAAAAAACATATTTCAAAAAATT
GGATTAAGAAATTTTTTTATATAAAATATTGAGTTGAGATGTAGAACTTGGGACTGTTTTGGAATTTTTT
TAAATATAAAAATATATTAAAATAATATTTTTATTTTTTTAAATTTATTTTTGACATAAACAAATTAAAA
CGATTCAAAAATACTAAAAAATTAATTTAAAATTAAAAAATAAAATTTTTCAAAAGCATGGTTAGACCAT
AAATGTTTAATTAAATCATTCATATTTTTAATTTGATTTATCTATGAAAATAGATTATATCTCGTAAATT
TGAAGCAAATATATATATAGATTTATGATTGTATTTATTTCAAATACATGAACCATGTTATTATTTTATT
TGATGATAAAATATATATTCAGACCAGTTTATGTGTCTTACATTTAGCATTTTTTTCTGAACAATTTTAA
TAAAAAACAAATACTCTGGAAGGATCCCAGCGTGTATATAATGGAGTTGAGATTTAAAATTATATTAAAT
AAGAATAAATACTAACATAAATTCATAATTCTTCTTATTGCCAAATGAACCAGCCAGTCACCCTTGGAAA
AATTCTGGCAAAAGTCATAAATCAACGAGAGTTAATTGGGCATTGAAAAATAAGTAAAGGATTGATCGAG
GTATATATATATTTTTTATTTTTTAAAATACAATAAAAATAACTCAATTATTCTTCAAGTTAAAGATTTT
GAAACAGATAAATAAGAGCATTTATATTTTTTTTATTTTTTAAAGTACAATAAAACTTTACCTTTGAAAA
AAAAACATGTGTATAAGGGGCCCTTGGGTCTTTTCATAATAATTTTTTTTTTTTGTTATTAATGGTTTTA
TAAAAAGTAATTTAATCTTTTTTATAAATTAAATATTATTTAAATTAAAAAACGTTTTGGTGCATGTGAG
CACTCGTGCATTTTTAGACGACTGGTATAATGTTTTTATCGTCTAAAAATATTCTTGGAAGTAATTTTAT
TTTATTTTTGATTGGTGTTTCAACTTTAATTTTTATTTTATTGATTTTATTTTAATTTTTAATTTTTAAT
TTTTTTATAAAAAATTTATTAGTTTCAGTTTTATTATTTAATATTAATTTTTTATGTATCATTTTTACAA
TTTAATCTTTATTTTTTTATTTTAATTTTTTATTATTAAATTATTATTATTTTCAATTTTATTTTTCAAT
CAAAATTTATTTATATTTTTTATTTTAATTATGATAATAGCTGAGACTTGATTACTAGAATTACATATTT
ATCCTTATAACTCAGGTTTAGTCATACCGATTTCTTCCGTTGTCTTACAATTTTCTTTAGCAACTTGGAA
AAAAATATCTCTGCTGTTTTGAAGTTGAAAAAAGAAGAAGAAAATGGAAAGCATCAGTAGATATTCTCCA
AAGTAAAGCAAGGCGTCAAGCAAGCAAATCCTCCACTTGCTTCACTGTCAGTCCAGTGGCACAATGCCCA
CCTGTTCCCTCTCTTCTCTCTTTTTGCTTTTGCTTTTAGCATTATTTCTCTGCACTTTTTCCCTTGGGAT
TGAAGAAAGAAAAGGAAGGAAAAGAAAGGAAAGTGACCCCTCTCCGTTTCACTCCCATCTCATTAGTCCA
GAAACTCCAACTTTTTCTTAATTTCTCTCACTTCTAATATAAAAGCAGTAACCACATTCTCTAGAGAACG
CACACGCTGTGAGTATACAACAGAAGAAACTTGGGTTTCTTTCTCTCTTTTTTCTTACTCCAGTTTCTTT
CTTCTTTGCTCTCAGTTTTGGCTTTCTATATTATCAGAATCTCAAGGTACGTTGTTTGTTTAAAGAAAGT
TTGTTTCTTGAATTGTTTGTAGTGTTCCTTTGCTTTATTCAAGAATTGGGTTTGCTTTAGCTTTTATTCT
TTCTTGGCTAGTATTAGTGCTTTTTGTTGATGGGTTTTACTTGGATCATAGTTTTCTTCCTTTAGATCTT
TTGTACTTGGAGGTCTTGGTGTTTTGTGTGCTGATTTTGTTGTATCATTGTTTTATCTGTGCCTTGTAGT
CTTCACAGTCAATGATCGGAGCATTTTGTGCTTAAATTTGTCAAGAAACTTGTTTAAATCAAGATCAATT
CTGGATTTATCGGAATCAGGATCATTAGATGCATGCTTAGTTGTTTTATGTTCAAGACATGCACTTTGGT
TAGTACAAATTGTACTAAAACTCTTAACTGCGTCTTAGGACCATTGGCCTTTGTTTTGGTGTTGCGATGA
TCAGGGAATCAGATTGTTTGTTGCTTTCAGTTGAAGTATTTAACATTTTGTTCAAAGTTTGTTTGTTGGT
GATGTGGAAAGGTCTGCTCATATTGTTAAAGTACAGTAATCAAGATTAATCATTGGTTTATCTAAATTAA
GATTTTATTTTTTTAATGCTATAGCGCTTTATGTTCAAAACTCTGCTGTAGAAATTGCACTAAAACTCTA
TTGGGAAATATCAATTTGTTGATGCTTATTTTATTGGGGTCTTCAATTTGGATTTCTTGTTTTTGCATGT
TGATCTAAGACAAATGTTTACCTTGATTCTAGATGTGAGATTTTCTGAACGCGAGTGTTTGCACACATGA
TTTGTGTTGTGCTCGAGAAAAATCAGTGACTGTTATGACTGGTGTGATCTTTATTATGTTCATTAAGCTG
ATCGTGGATTTGCATTAACCTTTGGGGTGGTGTAGGGATCATTGGCCTTTGTTTTGGCATGGCCATGAAT
AGAGAATTAAATTGTTGTGTTGCTGGTGTGCCCTTTGTCTTACTCAAGTTTGCTTTCAGTGAAAGTACTT
AGCCCTTCATTTAAGTTTGTTTCTCCATGATGTAAGCAATCATTTTCTTATTGTTAAAGTACAATAATCT
TTTGTCTTTCCAAAGACCAGTCTTTTCTGTTGAAGGATGAATTTATGGTTATTTTTCCAAATATTATTTT
ACCTTGCTATGATTCTTTGCCTTGACTAGATGGCACTACTTGAATTGCAGGATCACTATAAACC
Sequence ID: 7
Sequence Length: 2000
Sequence Type: DNA
Organism: Poplar sp.
TCACATTAACTTCTTTTGTAATTTACTTTAAAAAATAAAGAAGAAAAAATCTGTTGGAAAAACTAAAGAA
AATGAAAGAAAACGAAAAGCATCACTGGAGATACTGTCCAAAGTAAAGGAAGGAAGGCAAGCAAGCAAGC
AAGCAAATCCTCCACTTGCCACACTGTCAGTCCAGTGGCACAATCCTGTTCCCTCTCCTCTTTCTCTTTT
TGCTTTTGCTTTTAGCATTGTTTCTCTTCAGTCACTTCTTCCCGTGGCATTGAAGAAAGGAAGAAAAGAA
AGTGACCCTCCCCTCCCCACTTCACTTCAATCTCATGAGTCCAAAAATTCCAGCTTTTTCTTGTTTTCTT
TCACTTCTTAAATAAAAGCAAGCACATTCTCTAGACAACTCATTCACTGTGAGCATACAACAGAAGAAAC
TTGGGTTTCTTCCTTTTTTGCTCTCAGTTTTGGTTTTCTATTTCATCAGAATCTCAGGGTACGTTGTTTC
TTTAAGGAAAGTTTGTTTCTTGAATTGTTTATAGCGTTTCCTTGCTTTATTCAAGAATTGGGTTTGCTTG
CTTGCTTTTAGTTTTGATTCTTTCTTGCCAGTATCAGTGCTTTTTACTTGTGGATTTTAGTGTTTTGTGC
TCTGAATTTCTTTTATTATTTGATTTCTGTTGCTTTATAGTCTTTATTATCAATGATTGGATCTATTACT
TGGATCATTTTGTGCTTAAATTTATAATATCAATTGTTGATTATTGGAAACAGGAACATTCGATGTATGC
TTTGTGTGTGTTTCATGTTCAAGACTTGTGCTTTGGGCGGTAGAAACAGTACTAGGACTCTTAACTGCAA
CTGTCAGTTAGCGTAGTGCTTATTTGCCGGGTTTTTGGACATGGACTTCTTGTTTTCTGCTAGGACATAT
GTTTACCTGGGCTGTGGATTTGGGATTTGTTGATCATGAGTATTTGCATTTGTGGTTTGCGCTGCATGCT
ATAAAAATGAGTGATTGATATGACTGGCATGTTCTATTTTGTGCTCATTTAGCTGATAGGCGATTTCGTT
AACCATTAATATGGTGTAGCAATCGTTGGCCTTTCTCTCAGGATTGCAATGAACAGGGAATCAAATTGTC
GTCAGTTGAAGTATTTGACATTTTTTTCAAAGCTTGTTTCTTGATAATGTGGACAAGTCTGCTCTTATTG
TTGAAGTACAATAATCAATGAAGATATTTTTTACGTTCAAGACATGTAATTTGCTATAGAAATTGCACTA
AAACTTTATTAGGGGTCTTGAATTTGGACTTCTTGTTTTTGTATATGGATTCTAAGACAAACGTTTACCT
TGATTCTGGATACGAGATTTTCTGAACGCAAGCATTTGCACACTTGATTTGTGTTGTGTGCAAGAAAATT
GGGTGATTGGTATAACTGATGTGATCTTTAGGGGGTGTTTGGTAGGGGATTTATGGTGGTTAAATTTTAC
CCTTGTCACCAAATATTTTTTTTATTTTTCCACTAACTTTTAACCCTTCAATTTATTAGAACACCAAACA
TTCTTACAAACCAATTTTGCCAGCCTTAAAAATTGCTATTGTAATAATTACTCCAGTAATAATTCATCCT
TCTTTTTCATAGCCTAGCACCAAACACCACCTATTATGCCCATAAGCTAACATTGGATTGTGTTAACATT
TGAGGTGGTGTAGGGAGCACTAGCCTTTGTTTTGGTATTGCCACAAATAAAGAATTAAATTGTTGTATTG
CTGGCTTACACTTTGTCTTGCTTAAGTTTACTTTCAACGAAAGTACATAACACTTCGTTCAAGTTTGTTT
CTCCAAGATGTAAACAGTTCTTTTCTCATTGTTGAAGTACAATAATCTTGTCTTTCCAAATACCAATCTT
TTCTGTTGCATGGTGAATTTATGATTATTTTTCCAAATGCTATTGTACCTTGCTATGGTTCTCTGCCTTG
ACTAGATGGTACTACTCGACTTGCAGGATCAGTATAAACC
Sequence ID: 8
Sequence Length: 2000
Sequence Type: DNA
Organism: Poplar sp.
TTTGTTTTGTTCTTTAGGTGCACCTTTTCCTGTTTTGTTAGCATTAATTGAAATAAAAATGACTTTCTTC
TTCCAGTGGGCTTGTAAAGTATATTCTACTGTGATCATTATGAATGTTTTCTGCAAAAGAACTGTGACGA
GCAATGACATAGCTTTTTAGTTGAGTCCTGTAATATTTCTACATATGAGATGAGATTTTTTTCTTCCTGG
ATCAAATTAAGAATAAGCTAGTGAGAATCCTGGCCGAAGGTAGAAGAAATAGCACTGCTCTCTTTCATTT
CTTTTCCATTTTAGAAGCCCTCACTTCATCTCCCTTCCATCAGAACAGTTTTTGATCGAGGAAGATACTC
CAGCTATGTATATCAAAATGGATATATATATAATAGTTTATTTGGTTACCTTAATTTTCCTGACAGATTT
CCGCGATACATGGGCATTCCTAGATGATCGAGTGAAAGATGCTTTTGATCTCAAGAAAACCATTCAGGAG
GTAGTCTCTTTTAGTTTTCTATGGTGTTCTATATCAACTTATTTTGGGAATTTTGGCCTTTCTTGGGAGG
GTAAGGTAATGTGGTTACTTTACTGCAGGCAATGTATATGGCAGAAGCTGTTGGTGCTGGAATGGGGAAC
TCTTTCCAAGGATTTGTAAGGAGAGTTTTTCAGAGGTGAACTATGTATCAATGTCACACATTTTCACTTT
CTTACCCTTTAAAATGTTATCGCCTATGATGAGAGATTCAAACTAGGAGAAGATGTCTTCATGTTGAAGT
ACCTTTAGTGGTTTTGCAATTTTTACTCCTTTTAATATCTGAGGTTGCTGTCCGAGGTCAACCATGTAGC
GAATTTCCAGCCCTGTAGGAATTGATTATTTATCCTTTCAACAATAAGAAGTTAAAGATCAAAATGATCT
TACAGGAAATAAATTATCTCCATTTTCAAAAGCAACTACCTCGCAGCAATGATTTTTGTTGATTGATAAT
GAGATGCATTTATATTTACATTGCACCTTGGAAATTCACTTATCCTGATGATAGTTTGTTCAATCAGATT
AGCTGACTGTTTTGAATGTGATTGAGGTCAGACAGTTACGGGGGGGAAGAAGAAGCAGCTCCCTTCTGCT
TCCTGCTTTTTCCCAAAGTCAAATCTGTGTCTTCTCCTCACCATTTGGTCTCAGTACTCTACGGATCCAA
TTTCTCACATTTTCCTTGCCGCATTTATCACTTGAACACCGTATGCTCTTGAAGATACAAGCCAAACTCG
AGCCACAACAATTTAGCAACAATTTAGAAATCAGCTCACATTATTCACAAGTTTCAGCCCTTCATCATGG
TGGAACCTCTGTTTATAGAACCCTTTACTATCCTGGGAATGCAAAGTCTTTCAAAATCTCCAGTACTCTC
AGCAATTAACGAAACTCTGCTGGAGAAACCAAGAAGTCAACAGACACAGTAGTAAACTATAAGCTCGCTG
TTCAAGCAACTATTATTAGTAAATCAAGCAGAAATAAAACCCAGTTCAGACTTCGATTTCTCTTACTTTT
CAGGAGATCGAGAGTTCTTGTTTATTTTCTGTTAGGTCTGGTCATGCATGTGGCGAGAAGGTGTACGGTG
ATACGGTCAAGGCTTAATACTTACAACTGAATATTAAACATTTTAGCAAGTTAATTCTTGGAACGCCCGT
TCCTGACATATTCTGTACTGTGTCAACAATCTATCCATGGCCCAAAAAACAACATGATGTGAGCATAAAT
CAAGCAAAATCACCTCCGATGTGAGACGAAGGGTACGTAAAAGGTGCCTTTCAACCTAGTCTTGTAAATA
TGCTGACATTCACTGGAAGCAACCGGAAGTGGGTGATTCTAAGATTCATCCTAAAGTTAGAGTTTACCGC
CGTTCTCTTCCACCGCTTCCTCTTCTTAAATATCACACATGTCCGTGTACTCTCTACCTTCTACAGATAT
TCTCTGTCAATCATCTAGATCGAGTTTTCATAGCACAGGC
Sequence ID: 9
Sequence Length: 3000
Sequence Type: DNA
Organism: Poplar sp.
AACCAAAATTGACATTTTATTAAAAAAAAAGATGCTAAATAGCATGCCACATCGTGAGAGTATGTTTTTC
TTGTTTGCATCAATCGCTTAGCATTTTAATCCAGTGTTTGATCATCGTTAACAACTCTTTTAACATTTTT
TAGGTGTATGATGACCGATTAGTTTTATTGAACACATTGATCGAACTTTCAATACGCACTTCAATTTTTT
TATCATCAACTCGAAATTTAACATCTCTAACATGTACTTTTTTATGCTTGAGTTGACTTTTTTCTCCAAC
CTTCAGCATAGCGTGGGGCCAGTACTAGTTATTTAACTAATTTGTATGATTTTTTTTTATATTTCCTAAT
AGATTGTTTTGAACGAAGGAATACCTCTTTGACATATAACAATCTACTAGATCATTGGTTTGCGCTATGC
TATGGGTCAGATTAAATTTTTTAACATAAAAAATGTTCATGCATTGCTAATGTTTTTGCTAATAAAAAAA
AAGTTAATCATGAACTCAAAATATTATGCATGTTGTATGATATCTTTTGTAAATATTGATACGGTGATAT
ATTAGATTGACCTAGATTAACCCGTCAAATTTGTCATTCAGGTTATAAGATCATGATAACCTTATAGAAA
ACAAAAAAAAAAGTCATGATGGTCAATTTCTAATTCACTCAATGTTGAATGATGAAATTAAAAAGAATAA
ATAAAAAATGAACAAAAAAACTACTCAGGTCAACTTGGCAAACCAACAACCCGAGTCATGAGATTGAGAT
AACCCCGTAACATACAAATTAAAAAAATATTATGAAGCTCAATCTCCAACCAATCCAAATTTGAAGGATG
AAATCGAAAAAAAAATCAATTAAAAAAAAGATAGGTTAAAATCAAGAGAAAAAAACTGTATCCATGAGAC
CGGGATAACTCCATATATAATAAATTGAAAAACAAATACGAAGCTTAATTCTCAAACAACCTAACATTGA
AGATATTGAATGATGAAATTAAAAAAAATAAAAAAGATTAAGTTGTCGGAGGATGAAATTGTAAAAAAAT
CAATCTAAAAAGAACAAAAAAATGAATAGAGTTAACACGGGACAACTTGTCAAACTCGCGACTTGAGTCA
TAAGATCAGAATAACCCCATAAAAAATAAAAAAATAAAATTATGAAGCCTAATTTCAACCAACCCGATGT
TGAAGGAAGAAATAAAAAAATTAAGTAGAAAAAAACGGTAAAAAAATACCCGAGTGAATCTGGGTTAACT
TGTTAAACCTGCAACCTATATCATGAAATCGAGATAAATTCATAAAAATAAAAAAACAAAATTAAAAAGC
CCACTTCTAACAGATCTAATGTTGAAAGTTAAAATAATATAAAAAAAACTAAATCCATGAAATCAGTATA
TAACCCAACAAAAAATAAAAAAAATTATGAATCACAATTCCCAAAATAACCTAATATTAAATGATGAAAT
TGAAAAAAACCTAAATAAAAAAAGTCTAGGTTGTTGAAGGGTGAAATTAACAAAAAAAAATATTATTCTA
GTGAATAGTGTTTTATAAGTGTGACTATAGTGATTTAACCACATCTTTTAATGTATATTTGATATTCTGG
TAGTTTTTTGCGGTTACGATTTGAAAATGTATGTTTAAGAAAAGCTAGAAATTGTAGCTTTTCAGCAACC
AAGATTTTAATAAAGAATTTTCAAGAAAACACATGCAAGCTATGAAGTATGTTGATTACCAAACACTTTC
AAAACTGTGGTTGAGGCAAAACATAGTTTCAGGCTTTTAGCCACAATTCATCATACTACCAAACATATAC
TTAATATTTTGTTAATTGAATCTCTCATATATAGACAACATTTAATATTTTGCAAATATATCAAAAATTT
CAATCACTTAGGATGTTTCTTTCAAGGCTCTAATATTTTTTTTATAAGGGCGACTAGCTTTGTCAATTGC
TCGATAGTTTTTCCATCACATCTGATGATCGAAAAATTAGAGTTAAATTGATGGATTTACAAAATCATTT
TTCAACTTTTTTTTCATTAGAAAACGCCTCAAAAACCTTATTGAGACCTCAATAAATCCATTTTCAATCT
CAAAACACTCTTTATTCACTCCAAAAAAATGAAGATTAAACAAATAAAAAACCTTAACTGACACCGATCT
ACTTTCTTTGGCATGTTCATAGGAAAACATTGTCTTCATTGAACTCCTCTTAAGAAGACAAACCTATTGA
CTCAAAAATCAACCTTTTTGGTAGCCAAAATATTACAATTCGAGCACTTTCTCTACCCTCTAACATTTTT
CAATGACTTTCTTTCTCTTTTCCTAACCAAAATATGACCATCACAACACTTTGTCTCTCCTTTATGTTTT
TCAATGACTTTCTCTCTTTCTTTTTTTAATATCTAAGGATTAAAATGTGAATACGATAAAGTTTGATATC
AAAACATGAATGGTTAAAATAACTAGACTAAATGATAGAAAAATAAAACTACAGGGATCAAACTTATTGT
GGAATATCACTCAAAACTCAATACATGCGAGAACATGTAAATTATTTGTTTTTTTTTAAAAAAACATGCT
AATGTAACTTAGAAGAGTAAAATTGTAAATAATAAAACTTCAATGATAATTATTTATTGCGATCAATATT
TATTTTAAATCTAAATTAAAAAAATTAAATATACATAAATTAAGTTGAGAAAAATCGTGTTTGGTATTGC
GATCAAAATATTATATATTAAAAATTAATTTTATTTTTTTGTTTAAAATTATTATTTTTAATATTTTTAT
ATTATTTTGATACATTTGATATTAAAAAAAATAAAAAAAGTATTATTTTAATATTTTTAAATAAAATATT
TTAAAAATTAACCACAAAATATAAAGAATTACGGGACAGCCCAAGTCATCCCAACAACGTTCCTTAGCTG
ACTGCACTTTCAATTTATTCCAAAGTTTGAAGAAAATCATCATCGCAGAGCTGCCTCATC
Sequence ID: 10
Sequence Length: 2420
Sequence Type: DNA
Organism: Poplar sp.
AAAAAATGTATCTCTAACCCCTCTATCGATAAAATCGACAAGGTTTAGAGGCACTAATGCCTCCCTAAAC
TCTCAGGTAAAGGATTCACAATTTCTATTCTCTAGACTTTAATTCTTTTAAATCCCAGAGTATTTATTAT
ACAAAAAATAAGAAAAAAACACTCTTGTTCTTGTAATTTAAAGTTTATTATCTCGTTTATTGCAATCTTT
TACTTAAAAGTTACCGATTTTATCATCCAAGAATCTTTAAACCCCATCAATTTCTATTACCAATCATAAA
TTTCTATTTTTTTTTATAGGTGTTAGATCTTCTATTACCAATCATAAATTTCTTAAGCTCTGGATAATGT
GATATTGACATGAAAACATAATTATGTATTATCTAAACACCAAAACATTTTATCCCTAGGTATATTAAAT
TTTAGAACACCATGAATTACAAAACAGAATTGTAGAAACTAATTAAATGTTTTTAATCATTCTTAGCAAC
TATTTTAAGACATTCGTTAGTGGCTTATGATGGTTAAAGCTCGCCTTTTACATTGAACACAACTCCTTCC
AAAAAAAAGTTTTTTTTTACCATAACTAGCACAAAAAAAGAAGAGGGATTTGCCCTTTTTTTTAAAAAAA
AAAAAAAAAAAAAAAAAGAAGAAGAAGAAGAAGAAAACTAAGGAAAATCAGTAGCCTTTTTATCTTCTCC
TGTTCAAATTAAAAAGGAAAATAGAACCACAATTAGCGGTCGATTAATCCAAAAGTGATCAGCCACAAAT
AATCCGTCCCACGTTGTGTTTGTTATGCATGGCACGTCATACGCTTCATTTATCATGATACAATGTCAAC
CGTCAGATCCTTGACTGATTCCTCTAGTCAATGACGACTCCGTTGCATTGAAGGCGACAATCGAGGGTGT
AACAGACAGGTGATGATGGCCATAACACCACCTGAAAACACAGACCGACATGATCTCCAGACAGTTGGAA
ACGGAATGCTGAGTTGGCAATTAATGTTTTTTATTTTTTAGGGTTTTTTTAGTCCTTTAACAGAGACTAA
CTTGCTTCGTGATACAGAAATGTTTGAGAGTAAAATAATAATTATTTTTAAAAAAATATTATTTTTAGAA
ATATATTTAAATAATATTTTTTATTTTTAAAATTAACATATTAAAATAATAAAAATATTAATAATAATTT
TTAAAAATTAATATTTTTAAAAATACCGTTCAAAATATAATTTTAAAAACTCCTGTGTTCCAATTGATAG
GAAATTTTCAAGTATCATAAACTCAATTTACTACTTATAATAAAATAAAAAAAGGACAAATGTTAATAAA
GATAATCTAATGGTTTGAAGATGAGATTAAAACATAGAATTTCAACTTAAAATTTTAAATTTTAATAAAA
AAATTATATTATTAATTTTTATACGATCTGTATGATGGATTTGGAGTAGTAGAATAGTTGAGTTTTTGTT
AAAATAAGATATTTTATGATTTTCTAGAATACTTTTTATGAAATCATGATATGTAATAAATATTTAAAAA
TTATATTTCTTAAAATATGTAAGTTAAATAAATAATATTCACATATGATTGAGTTTTAGGCTTTGTAACA
TAATATTTTTATTTTAAAAAAAAACCTCAATATCAACGAACTACCCGTGAAAAGAGAAGAAAATTAGACA
CCAAAATGAGATAAGCAATGAAACGGTCAAATTGAGATTTGAGAGAGTGGTCAAAAAAATTTAACAAAAA
CCCACAATTAAAGGCTTGCAATACCCGAAATGCCCTTGTTTTACATCCGAGGTTAGAGGAGGCATTACAA
CAATCCCAAGAGCAAATCCGTCCATACGAACAAAACAACTGAAAGTGAGCCAGCTGTTTACACAATGAGC
GTGACGCGTCATGGGGTCCGTAGATCAGATACTGAGAACCAACGGCCAAGATCATTTCAAAGAGAATTTG
AGGTTCATTTAAGCTGAATTTCATTTGAGGCTGAAATTTCATTTCACATCTTCCCCTTTTTTAAAACTAG
AGGGCGGGTACCTTGAACAGACGCCCACACAGAGATCGAATCCAGATTTCTAAACGGTATTTCATTTGAG
TTTCATTATTTTCTTTGTTAATCTGGGGTTAGCTTAGAATCCAGCATTATCGATTCAAGCAATTAAAAGA
CTTTATCTTTAAATGTGGGCTAATTCAGTTTTGTAATTCAGCATTGCTAAGGTCTTGATCTTTGATGTGA
TTTTATCAGAATGAATATTCAGATTTTGATTGATGCTTGTAAAAAGGAAATGGGATATTCTTAATTCGCT
TTATTTTTGGATTTGTTTTTGTCAAAATAGTACATTAGATTGTTGTGATTCCCTATTTTTATAGCATTTT
AATGTAAAGTTGGCATTTTTTATGTGGCAGGTGAGAGGAA
Sequence ID: 11
Sequence Length: 3276
Sequence Type: DNA
Organism: Poplar sp.
TCCATTACTTGTTGCACATAGATTATGCTTTTGTTTATTTTGACAGTGATGAATTGTGGTGACTAATTTC
ATTTTTTCCCCTCATGCTTCTATTTTTCTTTTGGGTGCGTAGGATTGTTGAGAGTGTTGGTGAAGGTGTG
ACAGACCTCGAGCCTGGTGACCATGTGCTTCCTGTATTCACAGGAGAGTGTAAGGAATGCCGACATTGCA
AATCAGAAGAGAGTAATATGTGTGATCTTCTTCGGATTAACACTGATAGAGGTGTGATGCTGAGTGATGG
CAAGTCTAGATTTTCAATTAGAGGGCAGCCCATCTACCATTTTGTTGGTACATCAACCTTCAGTGAGTAC
ACTGTGATTCATGTTGGCTGTCTGGCCAAGATCAATCCTGCTGCTCCACTGGACAAAGTTTGTCTCCTCA
GCTGTGGAATCTCTACAGGTTGGGAAAACACCAATTCAATTATTCTTAAACGAACAAAGATTTTCTCACC
TTCTTTTGATGAGGATTTGGTTTCGTGTTGTAGGTCTTGGAGCCACCTTGAATGTTGCGAAGCCAAAGCC
AGGCTCCTCCGTGGCCATTTTTGGACTGGGAGCTGTAGGCCTTGCAGTAAGTTTCTTTTCCAATACACGA
AACAGATGATTTTTCTATCCATTTATGAGTTCTGATGCTAGCCTGCTACTTATGTTAAATTTGTTTTTGT
TGTTTGACATGATTAGGCTGCTGAAGGTGCTCGGATTTCCGGTGCTTCAAGGATTATTGGTGTTGATTTG
CATTCCAGTAGATTCGATGAAGGTAAAACAATCATTTAGTTTTATCATATCACTAGTTTTCTTTCGCTTT
TTCAGCTATTGCTCCATTGGTGTCTTTAGCTTGTCGGCTGCATTAGTTAATTATTATGTAAGAGAGGATG
TTGCAGAGAGGATTTTAGCATTGTCAACTTACTAGGATGAAACTGATCTTCATATTATCGAGTAGCTTCG
CAGTTGCTACTTGGTGATTGTGCTAAAATATTACACAAGACGTCATGTTTTTTATGTTCATATACAACTG
ATTTCTGATGTTCATCTGTGTTTTTTATTTCAACAGCCAAGAAATTTGGCGTGACGGAGTTTGTGAACCC
AAATGATCATGACAAGCCTGTTCATGAGGTTTGTAAGAAGATGAATCATGTTTGGAGGGATGATTCATAT
GAACCTTTTGCATCAGTTCTAATTTTGATTTTCCGTCTGTAGGTGATAGCGGAGATGACTGATGGAGGAG
TGGATAGAAGTGTTGAGTGTACAGGAAGTATCAGTGCCATGATTTCTGCATTCGAATGCGTCCATGATGT
AATTTTCTTTAAACTGTTTTAGTGATTATGGATCACTAAAAAAAAAGCACTTTTAAAACACCCAAATCTG
TTAACATTATGGATCTCTTTGCAGGGTTGGGGTGTTGCTGTGCTAGTCGGCGTGCCAAGCAAAGACGATT
CTTTCAAAACCAATCCAATGAATTTCTTGAGTGAGAGGACTCTCAAGGGTACTTTCTTTGGAAATTACAA
GCCGCGCTCTGACCTTCCTTCAGTTGTAGAGAAGTACATGAACAAGGTAACTTCAATCCCCATTAATCAA
TCTCTAGCCCGTAGAATTATGGTTTCCATTAGTGCTGATCGGAAGCAATCTGTATATGAAGTTTCTCAAA
ATATCTACTTGAATTATCATTAACAGTGACATTGTTTTATTTTAAGAACATTATCATAGGCAACAAAGCA
GCCATTCCTCCTCCTTTGGTTTTGAACATTCTATCATCATGCAAAAACCCTCTTATCCTAACTAGTTGTT
ATATGTGCAGGAGCTTGAGCTAGAGAAATTTATATCCCATGAAGTCCCTTTTTCTGAAATCAACAAGGCT
TTCGAGTACATGCTCGCTAAAGCTAGCCTCAGGTGTATTATCCGGATGGGTGCATAAAAGAAGAATCATC
TTTGTTGATTTTCAGGTGTATTGCCTGAGCTAGCCTCAGGCGTATTATGTTCCGATAGTGATCAGTTAGA
TGAAACAATAAAATCCATGCTTTAGATTGATAATTAAGTAAGCTGTTACCACTACACTTCCTCATGAGAA
TAAATAAGATTTTGACGTGTTTTTTTTACTAAAGGTTATCAGCACGTTCATCTATTTCACAAGAAGAAAA
CAAACACAGGAACAACACGTTTGAATTCCTCTCATTAGGCAAATCAGCTGGAGCCAACACTTTATTTTCA
AAGAATGATCAGGCGGAGCCATATTGGCCATAAGTGGTCAAGTTGCTAGAACATGTACTTCTTGAAAAAC
TCATAAAAGTGTCTGTCAACTTCTTCTTGTGTTAAAGGCCTCGTGAAAATACTATTTTACTCTTAAAGGC
CAGTTCTTTGTGATTTGTTTTGAACGGTAAAAGCATAATTAGGATGTGTTAAATAATAAATCCTGTTCCT
AAAGGATGATCCATTTACTCCCTAAATTATACAAAATTCCCGAGGTTTTTATTCTTCTTCAGTGAAAAAG
AAAAAACTGTAAGAATGATAAAGGGAATCGAAATTATGTAATGAATGATCCATTAGTACATTGTAATAGA
CTTTTTTCTAAGGCAACTTATTCTATTTAAATCAACAAAATCCATGTACTAATAAATTATACTATCAAGA
GTTATTCAAACTGGAGAGGTTTGGGATTATTATTATTATTATGAACAAATTGATTATTAGTGACAGCCAT
TGCTAATCAGCGTGAGGGTTTTGGGATACCACTGTAATGAAAGGTTGGAACAAGTCTTTAACAGCACAAA
AGATAGACTATAATTAGCAAAAACCACAAAGGCTAATTAGAGATTAATCCCCAATCGTGATAGAGATTTC
AGATTGATTTATCCACAAAGAGAAAAAGAAAAGTAAGCTGATTGGGTGATAAAATTGCAATAATTGCTAG
TTTTGAGACTGCATTAACACGAACATCTTAGTTGGAGGACTGATTTGTATTATCTAAAAAAAGAAACATA
AAACCAGAAACAGTCCAATCCTGAAACCAAGGAGCCGCAGGTTCTAAATACAGAGGACAGACAAAACAGC
AGAGATCGTCAATGGTTTTCTCTTTAAAAATCTTCAAATCGATAGTCAACGTTAAACATGAGAGACATAC
ATCCTTCTGCTACTATAAATACCCACAAGGAACAACCTCTTTCTTCACAACTCAAGAAACAGAGCAAACT
ACTCAAAGAAGCCTCAAAGAAAAAGCTGTTAAAGTTCGTTTAAGACAAGAAAGAAT
Sequence ID: 12
Sequence Length: 3140
Sequence Type: DNA
Organism: Poplar sp.
AAAAAAGCTCATGTGACGGTTGTGTATATGATCCTTAGACTTGAGATCACTAAGTATCTTATATAGAGAA
TGTTATGCTTTAATCATGTTACATGTTATCTTGATCGAGGTAACGAAATGACAGACATTGGGTATAACAT
GAACTATATAAAGGTACTTGAGTGATCAAGAGAACATTCATCACCCTAGGTGAATTAGGGAAAGTATTTC
ATTTGTTCTCAAATAGTATTGATTGAGAAATCATTGGTCAATGTGGAATAAGATTTAAAAAGAGTTTCAA
ATCTTATAAAAACGATCAATGACTATTATGTAAAGAGCAAACATGATTTAACATTGCAGATATACTATAT
GCTTTAATGTCAAATTAGAACATTATTTATGAAAGGATATTAATTACACTAAGAAACTAGTCACTGAAAA
GTTAGGTCAAACCACTAATGATTTTTTTAATATTCAATGGATCATGACATGTTGCTAGATGATGCACCTG
ATCTTCAAATATAAATTAATCAATTTTTTAATTGATAATAAATTAAATTGTTTAATTTATTTAATTCTAT
ATAATTAGAATTATAATTTATATTTGGGCCAACATATTAGGAACCTAATGGGTTACACGCATTAAGAACC
AACAGTCAGAAATTAAACTGAGATTATTTAATTAAATATGACTTAATTAAAATATATTTTAGAAATTAAG
AATTAGAATATAATTAATACAAGTATTATATTTCTAAATCTAGAAAAATCAAATAAGAACTTGATTGAGT
TAATTTTCAAAATTATCCTGAATTAATATATAGATATTATTCAGGGGCAAATTGACATTTTGTTAATTTA
TATGGTTTTTCAAATTTTTCTATAAATAGTAAGCTATGTCTCTTATTTTATAGATGTTTTATGTTAGACA
GAAAAACTACGTAAGCACAAAATTAGAACTAGCACTCAGGCATAACAATCCCTTTCTCATAAATAGGAAT
TTAAGAGATTTCTCATTGATGGTTCATGTGGATTGTTGTTGAAGGCCGGACAATTGGATAACTTGTGATT
TACGACAACCCAACTTTTAAAGAATTATTCAAAGCTGAAGAAGATCAAATCTTCAGGCAATAAATTCATA
AACAACTCTAGATCTATCTAACGAGATCCTAGGGACTCTCAAATAATATTGTTTCCTAGTTTCTGCTATA
TGTATGATATTTAGAAAACCCAACACCCGCTGCAAGATTAGTATGGCTATCGACCTCTTCGACAAATCTG
CCTACACAAAACAAACACAAAATTCTCTCTCTCCTAATCTAAGAAAACAACAACTAGTTATCAAGCAAAC
CAAGAAAGTGCTTGGCTGGAACATGTGTGGTGTGATTTTCTCTGTTAACATTTTGAGCATGACTAGAATT
TCTAGGGCTGAAGGAAGTTCTTGGTTGTTGTAAATATTGTTGGCATACATATAAATAGTGTAATAAGTTG
AATATTCATGATGTAACCCACAAAGGCACTATTATTTTGAATGGGGTAAACTTTGTTCTGTCACTGAGTG
AGGCAAAATGGTTTTAATTTTACATACTAATCTTCAAAAAATTCATATTAGAAATTATCTTACAAGATGG
ATTGATGATTGCTTATTTTACTTGTTAGATTATTATGTGATTGAGTATATTAAAGAGAATTTAGTTATGT
TTTTGTTATTTTTACATTTCAGAACTCTTTAGCCATTATTTCAAATATAATGAAGTTTTGTTAATAAATA
AATATTATTTGTTGACTCTTTTTGTGACACCAACTCTAAAGAATAAAATGTTTTTTGTCTTCAAAATTCA
AGTTATAAATTGTAATATTCAAAAACCTAGTTGCAAAGTTAAAAAAATAATCAAACCATAGGATTCTTAG
AGTAATTTACCCAAATTTAAAATGGAAACAAATTAAAGAATTTAATTGGGTTTTGAATGGGTTATCTCTG
GACTGATCGAGTTGCAAGTCAATCTAAATTTTTTACCAGATCTCACAAATCAATTTTATTCTTGTTATTG
TTGAAGCTCGACTTAACTTAAACTTCAAGTCATCTAAGTTATGAATGATCCAGTCAGATCAGATTAGACA
CTTTTTGAAGAATTGTAATTGCTTTCGATTACGTGTAAATAAGTCTTATCATATATTAATTGATTATTTG
TGTTTTGAGTGAGGATTTATAATCTTTATTTTTTATATAAAAAAATGAAAATAAAACGTTTGCAAAATAA
ACAAAAAAAAAAATTCTCAGGGCATTTTAAATGAGACGTTTTAAATTTTTTCCAGAGTAATTGCAGAAAA
TGTCTGTGTTTTCTCTATAAAAGAAATTCTTTTGACAGCCTGCACTTCATTAAAACAATAATAAAGGTAA
GTGTTTGTCCGGCTGTATTACAAGATTCTAAAAGGTCAACGACTTGCCGCTGCAGTGTAAAGGAAACAAA
CAGCAAAACCACAACTGGAAATACAAAATCTAGAAACGCTAATTGCAAAACAGTCCTTCAATTATGATAG
ATATTTCAATTAAATGTCAAACTATATACTCATTTTCAATTTTACCATCAAACGATCAATCGCTTAAAAA
TAATTTCCATACCATGTAATTTTTCTTTTGAGTGTCAAGAACTACTTCTATCGTAATATTAATTAAAAAA
ACACTACACGATAATTTTTTTATCACAAAATAAAATAAAACTTCTAGTTAGTTTAAGAAATTGTGAACTC
TGCCAAATGTTTGTCAAAACTACAAAACCAGTGACATTTGAAAGATAAATTTGCAAAATTGTTTCTTCAG
ATAGTATTAACAAAGTATAGCATAGTTTGAGGACAGGTTTGTAGTTAGCCAAAACCAGACACAAATTAAA
TAGAGAAACCAGAAAGCCAGAAACAGGGGGAGCCGTTGTTGCCAACAAGAGAGAGAAACAGAGGACAAAT
GAAACAGCAGGATCGTAAATGGTTTTCTTTTAAGAAATATTCAAATTTATGGTCAACATAATCCAAAAAA
CAGGAGAGACATGCGTTCTTCTGCAACTATAAATACCTAGAAAGACAACCTGTCTCGTCACAACTCAAGA
AACAGAGCAAAGTAATCAAAGAAGCGTTAAAGAAGGGTCTAAAGTTCGTTTAAAACAAAG
Sequence ID: 13
Sequence Length: 3140
Sequence Type: DNA
Organism: Poplar sp.
AAAAAAGCTCATGTGACGGTTGTGTATATGATCCTTAGACTTGAGATCACTAAGTATCTTATATAGAGAA
TGTTATGCTTTAATCATGTTACATGTTATCTTGATCGAGGTAACGAAATGACAGACATTGGGTATAACAT
GAACTATATAAAGGTACTTGAGTGATCAAGAGAACATTCATCACCCTAGGTGAATTAGGGAAAGTATTTC
ATTTGTTCTCAAATAGTATTGATTGAGAAATCATTGGTCAATGTGGAATAAGATTTAAAAAGAGTTTCAA
ATCTTATAAAAACGATCAATGACTATTATGTAAAGAGCAAACATGATTTAACATTGCAGATATACTATAT
GCTTTAATGTCAAATTAGAACATTATTTATGAAAGGATATTAATTACACTAAGAAACTAGTCACTGAAAA
GTTAGGTCAAACCACTAATGATTTTTTTAATATTCAATGGATCATGACATGTTGCTAGATGATGCACCTG
ATCTTCAAATATAAATTAATCAATTTTTTAATTGATAATAAATTAAATTGTTTAATTTATTTAATTCTAT
ATAATTAGAATTATAATTTATATTTGGGCCAACATATTAGGAACCTAATGGGTTACACGCATTAAGAACC
AACAGTCAGAAATTAAACTGAGATTATTTAATTAAATATGACTTAATTAAAATATATTTTAGAAATTAAG
AATTAGAATATAATTAATACAAGTATTATATTTCTAAATCTAGAAAAATCAAATAAGAACTTGATTGAGT
TAATTTTCAAAATTATCCTGAATTAATATATAGATATTATTCAGGGGCAAATTGACATTTTGTTAATTTA
TATGGTTTTTCAAATTTTTCTATAAATAGTAAGCTATGTCTCTTATTTTATAGATGTTTTATGTTAGACA
GAAAAACTACGTAAGCACAAAATTAGAACTAGCACTCAGGCATAACAATCCCTTTCTCATAAATAGGAAT
TTAAGAGATTTCTCATTGATGGTTCATGTGGATTGTTGTTGAAGGCCGGACAATTGGATAACTTGTGATT
TACGACAACCCAACTTTTAAAGAATTATTCAAAGCTGAAGAAGATCAAATCTTCAGGCAATAAATTCATA
AACAACTCTAGATCTATCTAACGAGATCCTAGGGACTCTCAAATAATATTGTTTCCTAGTTTCTGCTATA
TGTATGATATTTAGAAAACCCAACACCCGCTGCAAGATTAGTATGGCTATCGACCTCTTCGACAAATCTG
CCTACACAAAACAAACACAAAATTCTCTCTCTCCTAATCTAAGAAAACAACAACTAGTTATCAAGCAAAC
CAAGAAAGTGCTTGGCTGGAACATGTGTGGTGTGATTTTCTCTGTTAACATTTTGAGCATGACTAGAATT
TCTAGGGCTGAAGGAAGTTCTTGGTTGTTGTAAATATTGTTGGCATACATATAAATAGTGTAATAAGTTG
AATATTCATGATGTAACCCACAAAGGCACTATTATTTTGAATGGGGTAAACTTTGTTCTGTCACTGAGTG
AGGCAAAATGGTTTTAATTTTACATACTAATCTTCAAAAAATTCATATTAGAAATTATCTTACAAGATGG
ATTGATGATTGCTTATTTTACTTGTTAGATTATTATGTGATTGAGTATATTAAAGAGAATTTAGTTATGT
TTTTGTTATTTTTACATTTCAGAACTCTTTAGCCATTATTTCAAATATAATGAAGTTTTGTTAATAAATA
AATATTATTTGTTGACTCTTTTTGTGACACCAACTCTAAAGAATAAAATGTTTTTTGTCTTCAAAATTCA
AGTTATAAATTGTAATATTCAAAAACCTAGTTGCAAAGTTAAAAAAATAATCAAACCATAGGATTCTTAG
AGTAATTTACCCAAATTTAAAATGGAAACAAATTAAAGAATTTAATTGGGTTTTGAATGGGTTATCTCTG
GACTGATCGAGTTGCAAGTCAATCTAAATTTTTTACCAGATCTCACAAATCAATTTTATTCTTGTTATTG
TTGAAGCTCGACTTAACTTAAACTTCAAGTCATCTAAGTTATGAATGATCCAGTCAGATCAGATTAGACA
CTTTTTGAAGAATTGTAATTGCTTTCGATTACGTGTAAATAAGTCTTATCATATATTAATTGATTATTTG
TGTTTTGAGTGAGGATTTATAATCTTTATTTTTTATATAAAAAAATGAAAATAAAACGTTTGCAAAATAA
ACAAAAAAAAAAATTCTCAGGGCATTTTAAATGAGACGTTTTAAATTTTTTCCAGAGTAATTGCAGAAAA
TGTCTGTGTTTTCTCTATAAAAGAAATTCTTTTGACAGCCTGCACTTCATTAAAACAATAATAAAGGTAA
GTGTTTGTCCGGCTGTATTACAAGATTCTAAAAGGTCAACGACTTGCCGCTGCAGTGTAAAGGAAACAAA
CAGCAAAACCACAACTGGAAATACAAAATCTAGAAACGCTAATTGCAAAACAGTCCTTCAATTATGATAG
ATATTTCAATTAAATGTCAAACTATATACTCATTTTCAATTTTACCATCAAACGATCAATCGCTTAAAAA
TAATTTCCATACCATGTAATTTTTCTTTTGAGTGTCAAGAACTACTTCTATCGTAATATTAATTAAAAAA
ACACTACACGATAATTTTTTTATCACAAAATAAAATAAAACTTCTAGTTAGTTTAAGAAATTGTGAACTC
TGCCAAATGTTTGTCAAAACTACAAAACCAGTGACATTTGAAAGATAAATTTGCAAAATTGTTTCTTCAG
ATAGTATTAACAAAGTATAGCATAGTTTGAGGACAGGTTTGTAGTTAGCCAAAACCAGACACAAATTAAA
TAGAGAAACCAGAAAGCCAGAAACAGGGGGAGCCGTTGTTGCCAACAAGAGAGAGAAACAGAGGACAAAT
GAAACAGCAGGATCGTAAATGGTTTTCTTTTAAGAAATATTCAAATTTATGGTCAACATAATCCAAAAAA
CAGGAGAGACATGCGTTCTTCTGCAACTATAAATACCTAGAAAGACAACCTGTCTCGTCACAACTCAAGA
AACAGAGCAAAGTAATCAAAGAAGCGTTAAAGAAGGGTCTAAAGTTCGTTTAAAACAAAG
Sequence ID: 14
Sequence Length: 3000
Sequence Type: DNA
Organism: Poplar sp.
AAATGACTAAAGTAAACATTTTGCGTCAACTTTAAAATTGTCACTTGTTTTCTCTAATTTTTTTATTTTA
ATCCTTTACCTTTTAATTTGTTCCTTTCCAAAAAGTTCAATGAATAGATTGAAGGAAAAAGAAAATTGTG
GATTCCTCTAGACAACATCTATGCTTTTTAGGTTTTTCTTTTTTTGTAATTTGTATTTCACCAAGAGATT
TTATATCTGAATTTCATTTTTTTTATTGATAATATTTAAGTAGAATTAACTTATTTATATAAAAAAATAA
CATTAATGTTTAATAATTATTAAATAAATCAGTTTATCACATAAAGATATGGATCAACAGATCATCCTAT
ACATCAATCTTTTCACAAAAGTCTCTTGTTCATCTCTTCCCTGTTTCTTCCCCTGCCCAAGGCTATGACG
AGCTACTACAAGAAATATTTTCTGAAAAAACCAAAAACAAAAAAGAGACAAGACATTATTGTTAGTTGAC
CTGCAACAAATTAGTTACACAACGTATTATTACAATATAATGACGAGCTACTACAAGAAATATTTACAAC
TAGCTACATTACCAGCTCACAGGTAGGATAAAAAATAATCTAAAAATAATAATATTCAATCAATGTAAGC
ACATTTTTTTTTAAAATATCAAATAAAAAATAATAATATCAAATGATGAAAACAAAAGAAATAAGCAAAA
AAAAAATTATAAAATAATATCAACCCGTGTTAACTTATAAAACTCGTTCCTGAACCTTTTGATTGAAAAC
ACTAAACATGAAAAAAAAATACGAAGCCCAATTCCTAACAGGTTAAATATTAAAGGCTAAAATAAAAAAA
TAACTATACATAAGGATATGAAAGAAAAAATAATAAATAAAAGACAAAAAAGGTGTAAATCTACTTAGGT
TAATTCATCAAATTCAAGGGTTAAATCATGAGATCAGGATATTCTATAAAAAGAAAGACGGAGAAAAACA
AGAAGCTTATTCTACAAACAAAAAAAAATTGTCTAGTAATGAGATTAAAAAAAAAAAAAAGCGATGTGAA
TCTATGACCGGGCCATTTGACTACAAACACCAAACCTTGAAAAACCATAAAACCCAAATTTCAATAAATC
AAATGTTAAATGATGAAATCGAAAAAACAAAATTAATCACAAAAAAAAAGGTTCAAAAGAAAAAATAACA
TCTAAAATAATGAGGACCAAATTAAAAAAAACAAATTAGAGAATGGATAATGTTAGATTGAAGGGCTAAA
TTGAAAAGAAAAATTAATTTCATAAAATAATTAAAGAATAAGCCACCAAAAAGAATGAAAACCAAATGTA
TAAAAATAAATTTTTTTGATTAAAAGATGAGATTGAAAACAATTGAAAGTTTTAAAAAAATAAGGAACAA
ATGAGAAAACAAAGTCAGGACCAAATTTGAAAAATCTAAAAACAAAAAGCTTAGATTGAATGATATAATT
GAAAATAACAAAAAAAAAAACTTATAAAAGGGCCAAAGATAAATATTAAATAATTAAAAGAATGAGGACT
GATACTTAAATATACCCAACCAACAAGATTGCTCTAAAATTTTAAATGCTTGGTACGAATCTCAAGAGAA
AAGAGAAAAGAAAAGAAAAAAAAAACACCGGTGATAATCCGTCCATCAAAACCAAGTACCCACCATCTGC
TAAGGAAGAGTATGCCGAGAGATATTAAATGACATAGTAAAAGTAAATTTTTACCTTCTGTATGACGACG
TACACACTGCTGAAGTGATGAGACTTCTCTTATGCACTTAATAGGGTTTTTTTAAAATATTTTTTAATTA
TTATGAAAGACTAATTCACCCCTCAAAACAACTTTATTATAAAAAACAAATTTTAAATATAAAAATATCC
CCAAAGAAAGCTTTATTAAATTTTAAAATTATTTTAGGAAATTCACAATGCTAGAAAATATGAAATCTCA
AAAAAAAAAAAAACCTTGGAGACTTATTATCTATTTTTTTGCATTTAATAGTATTGAAGGGGTTTTACTA
TTAAAAAAAATCATTGTATCCCTAATCAAAACAATATAGACAAATGGATCACGTGGAGAATACCATTTTA
TCCCTAAGCTTAGTTTTCTAGGTTATTTGGTGAATGAAAAAACAATATAGGTTGGCCTGAGTGGAAAATG
TTCTGGTTGCTTAAGCAAGTGAATACAACAAAATTTCTTGGGAGAAGATCCCAAATCCAAGGGGAAGATG
AGTTCTTAAATGCAACTTCACTCTAAACTAATCCTTCTCCTTAAACAGATTGATTATTACTGGCTGCTAG
AGTCGGCATTGTATATATATATATGTCAACCAGCCGTTAACAAACCGCAAGATAAACTAAGAATAAATCA
CAAAACCACAACTGGAAATATAAAACCACCAAGTCTAAATAGAAATCAATCCCCAATCATGATGGCGGTT
TCAAATTGAGTTTCAAACTAGGGAAATCTTACTATCTAACTTAAAATCAAGCCCGCAAAGTTTAAGGTTG
CATATTACTAGGGAAAGCCAAGTTTTCTCACGCCTAGCATGTAAATTATATTCTTAGTATCAAAAACTAT
TTGATATCATGGAAAAAATAAAACTATAATCCATTCAAATAGAGAATTTTCAGTAAAAAATAAATGTAGA
AGGTAGGAAAAATTGAAATGGATGGTCACACTATATGTATCAAAATATGTTAAATAGAGGACTTAATTGT
AGTTAGCTCAAACCAGAAAAAACAGAAACAGGGAATCAAGAAACCAAGGAGCCGCAGGTCCAATACAGAG
GACACAACAAAACAGCAGAGATCGTTGATGGTTTTTCTCTTTAAAATACTTTAAATCAATGGCTAACCAT
AAGCATGACAGACACACATGCTTCTGCTACTATAAGAACCCACAAAGACAAACCTTTCTCTTCACAACAC
AAGAAACATAAAGTGCGTTCAAGTCAAGAAACATAAAGTTCGTTCAAGACAAGAAAGAAT
Sequence ID: 15
Sequence Length: 3145
Sequence Type: DNA
Organism: Poplar sp.
AGGGAAAAACTTACGTAAATCAATTTCCGGGGCCCTCTTGTGGACTTCTTGTTCGATCCAACGGAAAAAT
CCACAGGAAAAATCGATCATCCAATACTTTTATAACGAGCACTCCTTGACGTCTTTCGCCGGTAAGAAAA
AACTACTCCGCCGATATGGGTTGTTCGGATAAACGTGGATTCCATGAGCTGGAAACGTAAGAATTTTCTC
TAGGGGGTGGTAACTACAAGTGAGTCCCTGTTTGCAGTGAGGGTTTCATTCAAGTTCCTAACCAACAAAT
TAAGACACTTTATTTTATTCAATTGCATCCCTGTTAACATCAATTAGTCACAAGATTTTATTTTCCCTGT
TAACATCAATTAATCCCCCGTTAGCATTTAATTTTCATAGCAGGGAATAATATTGATATCATATTTTAGT
GAATTTTTGTAACTACTTGTGTATATGATGGGCAGTAACTTAATTTTGAAGTTTATAAACTTAATGAAAA
GTATCATTACAATTGGAGGACTTAATTTTCTATTAATTAATTAATTCAACTTTTAGGGATTTTAAAATAG
GAAAAGGGAAAGAGAGCGGGAAGAGAACGTGTATTGACATTCATTAGTTATGGGCCCACGGGTTGCTCTC
TTTTTTCTTTTCTTTTTTTCATTAAAAAAAGAAAATAAATTTTTTTATGGTCAATGAGTTGAAAATAAAA
TCTCCTAATATGACTTTTTATTTATATTTAATCAAGTCAAATAAAATATTATGAGATGAATATCTTATAT
CTATATTTGATTAATTAATTTAATTTAACTAAAAATAAAACATTTTTAATAAAAAATAAAATAAAAATTC
AAAATATTCTTGGTTATTTTTTAAATTAGTAAACAATCATATAAAAATCAAATAAAAAAATTAACGAAGA
CCAATGTCTAATTAAATAAACAATAAATGATGAAACTAAAAAAAAATATAAGCTTAGAAAAAAAAAACAA
ACCCTAAGAAAAATCTAAACTTAGATTAATCACCCAAACTCAAAACCCATGAAATTTTAGACTCATACTC
AATCAAAAAACTTGACTCTTAATCAATTTAACATTGAATGATGAAACTGAAAAAAAATATCATTTTTAAA
AAATCAACATAGTGGAAAAAATAACAATAAGAACAATAAGGATCAAATCTGATATGAAAATAAACCGAAG
AAGGATGAAAGCGTCAAAAAATTCAACTGTAAAAATTAATTCAAATAAAACAAATAACAATCAAAAGACT
GAGAATCAAATTTGACAGACAATAAAATTGAAAAAAAATGAAATTGAAAAAAAATCTTATAAACTATTTA
AAATAAAACAAATAGTAACAAAAGAACATGTATCAAATATGAATAAAAAACAAATTGAAGGGGTACTTTG
AGATTTTACAAGGGTGAGTGTAAGAATTGAGGATGAGAAAGAGAAAAAGAAGGCCACCAATACCAAACAA
GAAGTCCATTGATCACATGCATCATCTAGGAGTTGAAGGGCCAGTAGAAAGATTCAAATGCTGCCTTTGA
AGTTGACATTTTGCCGTTGGCCGACATCGTACGTTACGGTCGAATAGCACAATCACAATCATGCGTTGAC
GATATCCATTTTAATTTTTTTATAATATTTATATTTATAAAATTACATTAACATGACAATCACAAAAAAA
AAAAACCAGAATAAAAACACAAAAAAATCCTTTGATGTAAGAAAATTCAACTTTAATGTTATATAAATGA
CTATACTTGAAAAATAAAAACACAAAAGCACTTCGAATACCAATCTTAAAATAGAAATTGATTCGAAGGT
GATGTAGATATTTTACTATGCTATAAATGTAAAAATACGGAACTATTCCCCCGTAACACTGTAACGACAA
ATGAATTATTGTGAATAAATCATTTCACCTCCCTCAACTAATTATTCGGTTTGTTTTGAGAGCGACAAGA
AGATTTTTTTTACTGTAGATTACTATAGCTGCTGTCCCACGACTTTTATATTTTCTTATAATTATAATAT
GGATAGACCTCAGACAAGGAAAATCAAACCAGCATATGGTTGTGTTTTACTAAAGTACCCACCGCACCTC
TAACAAGACAGATGCATTAATGACAAAACAAAACTTAAGATTTGGATAAAATATATATAAAAAACGTTTC
TAAAATTAATTAAAAAAAACGATCCTGAAAACAAATAGAAGAGAAAATTAATTACGCGAAAATTGTACCT
TGACAAGAGAACGAAAAATCAAATCCACAAGCCATAAACCAAAAAATTAAAATCAAACATATTAATTAAT
TTTTATGATATAGTCGCGCCGAGGCTCCCGAGTTTCTGCGTTATTAGTGTCTTGTTCTACGAGATGCTTT
TGGATAGGATTTTGCTTAAACAGTGAATATAAAATGATGATTTTTTTTTAATGGTTAATATACAATTCTG
TTTTTCAGTATTTTTTATTTTAAAAAACATTAAATTAATCTTTTTTATATAAAGATATTAAAAATAAAAA
TAAAATTATTTTAATATATTAAAAATATTTTTTTAAAATAATATAAAAATATATTTTTAAAACATAAAAG
TAAATGAGCTATCATATCATTATCAGACGCCGATCTAGACTTCTAGTCTACTAGTCTACTGCTCTACAAT
GACAGATCCTAGAGGGGGAATGTACACACACGAAAATCCCAGCAGCAGGGGCGCCGAATGGTGTCCTGGC
TCAGTGGCCTGTAATTTTTTAATCATCATCCTTCATTATTAATTCTTGAATCCGTTTGTTTACGCTGCGG
CTGCGTTTTAGATAAATAATTTCGAAATCCCTGTTGATGATAGCTGTGCTGCTCGAGTGCTCCACTGTCT
TTCCAAAAGCAAATTCACCGTCGACTTGAAGGCCACGTGTGACCATAAATAAGATTAAAAGATAAGACAC
AGAAGAAAATCTCCCAAGTAACTACGAAACGTTAGGCGCCACCAGTAATCAAAACCACTTGACAAATTCC
ATCAGACCACTGTACTGCTTGCAGAGTTGTAGTCGTCATTAAGTTCACTATCTCTATAAATCAAACCACG
AGACGAGATAGTTGCAGCGATCAAAACACATAATCCATCATCTCAGAAATACAAAAGAAGAAGCC
Sequence ID: 16
Sequence Length: 3000
Sequence Type: DNA
Organism: Poplar sp.
ATGAAAGATATATTTTTTCATTTAATTTACTAAATTATTTTGTATAATTTCTTAAATTATTTACTAAATA
AGGCGGGATCTAAAAAAATGAGGAGAAATTATATATTTAGAGTTTCCATTCCCATTGAACCATAGTTAAA
TCATTTTTTTTTCTTTTATATATGTCTAATGGAAATCAGCTTCTATACATTAAGATTTAAATTCATCTCC
ATTCTAATCAAAGATATATTATCTAGGTTAAAATAAGAGAATTCATGATAATTTTATTTCTTAAATCGTG
ATTTGAGATTTCAATTCTTATTAGTTAAAGAGTATTATTGGTAATGTTTGGTTGCTTTCTCGGAATAAAA
ATGAATGAAGATAGTGAAAATAGAGGAAATATATTTTCATGTAATTCATGTTTTGAGATTTCAAAAATTA
ACTTCAAAATTATTCTAGAAACGTATTTTTTATATATTTGTGTCTTCATCTTTTTTTAATTAGACACATT
TTCTTATTATAGTTTTAAAATATAACTCGGGGTTGACCCTAGATAAAGAGTCATGGCTTGGAGGATCGTT
TCTAAAAAAAATTAAAAAAATAATAATCAAAATGATGTCGTTTAAACCATAATTGTTTTTTTTTTAAAAT
AGTCAGCAGATTGATGACCAAGTTTTGATCAAGTTAGGCCTAGGTCAACTGCAGTTTTTTACTAGAATAG
CTGAGGTTTTTTTTTTCTTCAATTCAAACTAGTTTAGATCTTGAATCAAAACAGGTTTTGGATTAACCTA
CTACTAGTACGAGTTTTAAAACACTAGTTTTTATTTCTTCTATTACTTTGTTCATGAACATTAACTAAAT
AACACAACCCCTAAGGTTATGGTGAAATCTCTTAACCGTTTGGGAACGCGGTTGAAACCGCGTTCCCTAA
ATTTTTTTTTATGTTAGATTATTTTGATGTGTTGATCTTAAAAATAATTTTTAAAAAATAAAAAATATGA
TTTTGATGCATTTCAAAGCGAAAAATACTTTAAACCGCAACCGCTACCACAATCTCAAACAAGCCATAGC
AAAAAGGCAAAAACCATGTCTTGGTTTTTTAATTTTAAAGTTGGAGTGGATTTGGGGCGAGACACAGTAT
AATTTTGGGTCGATTGGTTTTGTATTCTCTTGGGTGGGTCCATTTACTATGCTAACATCATCACAGAAGG
CCTTATTAATAGGTTGTATATTCTATGTGTAGCCTCCACTCCACTCCCACTCACAGCACACGACCCAGAA
TTTGGGCCCGCTGAGTTTTTCCTTCAAGAAAACAAAAGTGGGACCCTAAGAGTAGCTTTTGAGAAAAAAT
GTTCGATTAGTTGAATAATTTTTGTTTTTTTATATTGAAATTTATATATAATTACTTTTTAGATTCTTAT
TTTATATAAGTTAAAGATCTGTTTTTCATTTATTTTATATAAAAAAACCCATTTTACAACACTTTTAATT
AAACATGTCTTTTATTTATTTTTAAATCCATAACTAAAAGTATTTTATTTAAATCTATTTTTTAAAATTA
TAATCATAAAAATTATAATAATATCAAATACATATTACACGCATTTCAAGCTTGTTCATCAATATCCCAT
TTGAGACTCCATGCAAATGGTATTTATAGTTGTTAGACTTGATATAAAAAGAAAAAAAACTTAAATTATT
ACATTATGAGATCAATTATTGGATAAACCAAAATCAATTATTTAATTATTTTTTATATTAAATTAATTAT
TTTTAACTTGAGTGAAAAATCAATTAAGTTTGGCTAGATCAACTTTTATCAAATTAATATCTTGTTTGAT
TTAATTAAAAATCTAACTCGAGTTAAACTTTAATTCTTGTATTTTTAAAAGTGGCCATATCATATCTAAC
AATTATAATACTTACCTTCGACTATGTGGAAAATATTTCTTAGAAAACTAATCCTTAAATGAAATTCAAC
ATTTAATGGGGTCCTTTTTTTTTTAGTAAGATTAGGTTTCCATCTTACTAGATTAAATAAATACACATAA
GAGACGTGTGGTTTGTTCCAATTGGACGAAGATCTAATTTGACCTAATCTATAAAGAATTCATTCGGACA
TATATATTCATCTTTCTCGATAATCTTTTGCTTTAGTTGATAAAAAAAAAAAACCCTTTAATTTATGTAA
CATTTATTTAATTAGACAGAGCCACATCCTTTAATTATGACAATAACCATCTTCTTTGGATGCCGCTTTA
TGAGTTACGCCACCTTTTTATCTGCAGTGAAGCCTCGAATCTCTTTAAAATGGAAAACCAGGGAACAATC
ATGCGTGTTCCATGGGAGCCAAGACATGCAATGGAGACCCTGGATTTGCTTGCTTCCATCTATTCATCAC
ACCCTACATCTCTTTTCCCTTTACAGCAAACGAAACTAGCGGTGATCACTGACTGGTAATCACAAGCCCA
CATAAGCTCTGTTTTTATTAAGAGAATCAATCTCGTGCCCACAGGCCTCATCCATCCTTTGCTAAAATAT
TTGTCTAAAATTTATTTTTGATATGTGGGTTGAGCAATTTAGGAAACAATTTTCTTTTTTTTTTTATGTT
TAACAAAAGAGAAATAGTACACACACGTATTTGGGAAAAGAATAAAAACCATGTAGGCACGCAGAAAAGG
AGATTCTAGCGAAAATGTGATGAGGAAGAATCGAATGATTCTCCATAATATGTGCTGCATTCATTTTCTG
TTGCACAGATGTTATGGAGGATTTCAGCCATTTACTATTGTTTGTCGAACATCTTTAAAATGTATTTGGA
ATTTCTCATAAGAATATAGCTGGTAGAAAATTAAAGGCCAACTGGAGTACTAGATTCTGCCGCTATAAAT
AGTTAAATACCCCACAGAGCTACTTCAGAACTCAAGAAACAGAGGAAAGTAATAAAAGAAAACAAAGTTT
GTTGCGTCAAGAGAATAACGAGGAAGAATGGCAAGCACTGTTGGTCAGGTTATGCGAAGA
Sequence ID: 17
Sequence Length: 3086
Sequence Type: DNA
Organism: Poplar sp.
ACAATCCATCTAACCGTAGACTAAAATATATTATGTTTTATGATAACTCTAAGAGGTAACTCAACTGATC
AGATTTTTAGTTTGCTCTTTAATAATCACGAGTTCGAGTCCTTTCAGGGCCACTAGAGGCTTACATGACG
TTAATTTTAGGACCGTGAGATTAATCGAGGTACACGCAAACTGACCCGGACACCTACATTAATACAAAAA
TATGTTTTATGATGATTGAAACTAATTAGAAATTCATTATTAATAGAAAGCATATAGTTTGAAGAAATTA
TCAAACAAGATAAGATGGAATGCAAAATAATCCTTATAGGCAATAAGCTTTCAGCTACACTCAACATGTA
CAAAATAATCCCATATATAATTAACTTCACTTAAAGAATAATATCCATAAATTGATAAATATGCAATGAG
ACTTGAACTCAAAAGTTCATCTCTCTTAATTCCTAAGAGGAGCAGTTAACTTTGGATAAAAATTGAACTG
ATCATCATTAAAAAAACTTAAAAACAATGAATATTTTGAAAGCTCCAGCTCTCCTCGATCGAATCAGTAA
CCTTCTTGCACACTACTACCGGACCTGAAGCGTATTGGCCAAGGATACTATTTTGTAAAGGATAGATTGC
ATGCCGACACCCTCTATTTTGAACTGAAAGATTTTTGTTTTTTGTTTTTTTGTTTTTAGGTGAGTGTCTT
TATGTATTAAAATAGCAAGCTATATAACTATTTAGATAGTGACTCAGTGGTAAGAGCTTGAGATCAAGAG
GTTTGCTCCCTCTGTGGTCTCAGGTTCAAGCCCTGTGGTTGCTTATATGATGACCACTAGAGGCTTACAT
GGTCGTTAACTTCAGGGCCCGTGAGATTAGTCGAGGTACACGCAAGCTGACCCGAACATCCACGTTAAAT
TAAAAAAAAAATAGCAAGCTATATTCCATGAACAGGTAGCCATTTCCATTACAAAATTCAACTAATCCAC
AGCAAGTGGACTGTGGGACTCATCTTAATTGAAATTTATAAATGTTCGCATGTTACGATCGATCCATGGA
AGAGTAGTTAAGCAAAATTGAAAGATGGAATTTAAAAAGAAAAAAAAAAATAGTGCAAGCTCCTTCACTG
TAATATGAAATAGCTGGATTTCCCCATCATCAATTGTCACTTATTTATTTATTTATTATTTTCTTAAAAA
GAAAAACCATCTTGCCATTTGCTATCTAAATTACAGCGCCGAACAAAACAAATCACACAGCACATTGGTA
CTGAACGGGTCCTTCGTATAAACAGAAAATGATTTTAAAAAGGGGAAAAAAACACTATAATATTTCTTAG
AACAAAAAACTTAACATCTACGAATAAAATAAGCAACCATTTGCTCTAGTAGATGAGATATTTGTCCTGC
AAGGATCTGGCCTTCTCTGAAAACGCCGCGTTGTGTGTTTATACGCCACCCTGCTAGGTGCTATCTAGAG
CGAAGCCATGAGTCCCCATTGGAAAGATCTAAAAGGTGACCAAACACTCTTGCATAAACAAGTCACTAAG
GACGAAGCCATGAATCTATAAAGAAAAAGAAAACCAACAAAACCAGCTGTAGAAATGGAAAACCAGTAAC
CTTCATGCCATGGCGCCAATGAGTCTGCTTCTACCAAATCATTGCCACTAATTCCAATTTAGATAAAATA
AGGATGGAAATTTAATTCATACCCGTTCCTACTACGTCAATACCGCCACACGGTATTTCTAATTTAGATA
AAATAAAAATAATCAATAACCATGGAATACAATCTGAATGAGTTGATATTTTTTAAAATTAATAGAGTAT
GAATATTATTAAACTCGAACCAAAACTTAACCCGGAATATATATATATATATTTTTGTAAATTATCTAGA
TTTTTTTTAATACAAAATAATATATACATATTGACTTAAAAACATATATATAAATAATATTTATCATTTA
ATATACATATACATACTTCAAATATATATATATATATATATATATATATATATATATTAACTATTTTATT
TTTTTATTCATAAATTATAATAGATAACAAATACTCATTGGATAATAAAAATTTAATGAATAGTTTATAG
ATATTTAAATTTCTTACTTGATAAATAATGAAGAGAGTATAAATATAAAAAAACCCAACTCATGGGTACT
CATTATATCCCTAGATACAAACAAAACAAGCATAATACTAATAAGATTCTTATAATTTTTTTTATATCAA
AATAGATAGTTTCGTTCTGTTTTAATTGAATTTAAAAGGATAGATGTGGTCCTTGTATATATATATATAT
ATATGAATTATGGTCTGAAGATATTTTAATAAAATTAAAAAAATTGATTAGTCCTTGTATATATATAAAA
TACGGTTTGAAGATATTTTAATAAAATTAAACAATTTTGATATACGCTTGATATATAATTAAAACATTTT
TCGAAAATATTTCTATCTGAAAAGGAAAACAACATATGTTTTCGGAAAACAAAATGTCGTGTACAGGGAA
AGATTCAAAATTAGAGTTTTTCAAGTCTAAGGTAAAGAAAAATCATTGTTTTATAAATTCTGAAGTAATT
TCTAAAGTTGTTTAAATAGAAGTTAAATCAACCTAAATTAAATTAAAGAGAAATCTTTTTATAATAATTA
GAAAAAAGAATTTAAAAACAATATTCTCTAAACTAAACTTGAATATATTCCTGACTTTGCCTGACCTAAA
AAATGGGGTGAGATAATCTAATTATTAAATAAAAAATTATAACTATTTTTATATAATTGTTTTTTTAACC
TTGATCCTATCCATCTGGTCTGTATCCGCTATACATTCAACGTAAAAAATGCAAGAAACTGTTTCCCAAA
GACTTCCAAAGGGCAATGCATTTCAAAGGCACAGTATTAGGAGGGTTTCAAAAATTTTGATTAATTTATT
TTAATGTATTAATTAATATTAAAAATAATCCACTGTCCCAACACTTAGAATGCTATAAATACCCCTCAAG
AACAAGAGGAAGCTAATCGAAAGGAACTATTTGTGACGTTAAAAGAAGCTAGAATTTTCATTTTAGGACA
GAAAGA
Sequence ID: 18
Sequence Length: 3027
Sequence Type: DNA
Organism: Poplar sp.
AAGTTAGGAAGACCATGTATGCTTATCAAAGAATTCATTGTATGGCTTGAATCAGTCATAAAGGTAGTGG
TATAAAATATTTGATACTTTTATGCTTGGACACAGTTACTTTAGAAATGACAATGATAGTTGTATGTATT
TCAGAAAGCTCTCAAATGGTTCATTTATGTACTTGTTAATGACATGTTGATTACCTCCAAGAACATGTAT
GAAATTAACAATTTGAAGAACTAATTGAGTGGTGAGTTTGAGATGAAGGATTTAGGTACAACCAATAAGA
TCTTAGGTATACAAATATGCAAAGATTGAAGTCCTAACAAGTTATACTTGTCACAAAGAAAGTATCTTAA
GAAGGTACTTGAATGTTTTGGTATACAAGATTGCAAGCCAGTTAGTACTCTACTTACAACCCATTTTAGG
TTATCTTCAACTTTGTCACCATAAACTAAGGAGGAAAAAAGCACATGCTAAATGTTTCATATGCTAGTGT
AGTAAGGAGCATTATGTATATTATGGTCTATACTCATCCAGAAATTTCACATGTTGTTAGTGTAGTTAAC
AGGTTTATGATAAATCCTAGTAAAGTTCACAGGTATACCATGAAATGGATGCTCTACTATCTTAAAGGTA
CTACATATATTGGTTTGATATATAACAAGAGTAGTGGCACTAGAAGTAATGTTAAGGGTTTTGTAGACTC
AGATTATACTAGTAGTTTGGATAGAAGAAAATCACTAACAAGTTATGTTTTCACTATTTTAGGGTGTGTT
ATTAGTTGGAAAGAAATTTTGTAATTCATTTTTTCCTTGTCAATAATAGAGGCTGAGCATATGACATCGA
CAAAAGGAGTGAAACAAACTCTATGGCTAAGAGGTTCGGTTTGTAATCTTTGTTTATCACACGAGTTGAT
CATTGTGCATTATAATGACTAAAGTGTTATTCATCTAGCTAAGAACCAAATGTATCATGATAGGGTTAAA
TATATTGATGTCAAGTTTCACTTTATTCTAGATATCATGTTGTAGGATGTTATATCAGTGAAGAAGATTG
TTATAACAGAAAACCCTGCATATATATTGACAAAGCCTATCTCTGCAGTCAAGTTTAAGCATTACTTGAA
CTTAATTGGTTTGTGTAGTACTTGAGAGATGCTTGGTGGAGGCAATGAAAGAGGAGTTTAAGTTTCATAA
GTTGACTCCATTTAAGCCAAGGTGGAGATTTATTAGGTTTATCTTAAATTGTAGCAGCTCTATGGGAGGA
AATAAATACCTATGTTAACCTAATCCATATAAAAAATCTAAAGTTATCATATTTACAAGTCAAGGCTTAT
TTGTGAGTGTTTAAATTCTTTATATTTTTATGGCTAGATAGAAGAGAGAAAATATGAGTTTTGAGTTACT
TTGGAATTTGAAATATTTTACTTTTTCAAATGAATTTTTCTAATAATCTCATCCACAGAGTAGATTTAAG
TTGCCAAACTATAAAAATTTTATTAGCTCCTGTGTAATTAATTATGTTTATTTTTTTATATTTTTCTTAA
ATTAATTTGAAATTTTAAGATTGTTTTAATTCCCAAGATTTCTATTGTGCTTCACTTTGATGACATAATG
TTTAGTGGAGTTAGCCATGGGAATTGAAAGGTTTCCTAAGAGGGACTTCATAGGAATTAATAGACCATTG
TAAAACTTTTAAAGAGTTAAATTATTCATATATTATCTAACCCAAAAAAAAATGCTTAAATACTATTTTT
CATCTTTTAATCGAAAATTTTCAAAATTAGATGAACTAAAGCTGTTTTACAAATGATTAGCCTCTTTTCA
TTCAAAAGATATTTCAAACTTATTAATTATTTAAAAGAGAAAGAAAAAAAGAACAAGAAGAAACTATTGT
TAGTTGACCTATAATAAATTAATATGTATTAATTATATAAGTCAACTTGCAGTTAACAAGCCAAAAGATA
AAATAAGAATAAACCACAAAACAACAACTGGAAATATAAAACCACAAAGGCTAAAGAGAATCAATCCCCA
ACCATGATAGAGGTTTCAAATTGAATGTCAAACTACAAAATCTTTCAATTATACCCTCCTACTATCATCT
CTTAAAATCAGTCCCTAGTTGCATCTTACAAGGGAAAGCCAGGTTTTCATACGCCTAGCACATAAATTAT
ATTTTGAGTGTCAAAAACTATTTGATATCATAGATAAGAATTTGTTTTAAGAATATAATTTATACAAATA
GAGAGTTTTCATACGCCTAATGTTTTCTTCGCAAGCTATATGTTTTCTGGTAATATGATGATGTTGTTTC
AAATGAACTAATTTGATTACCAAACAAAGTTGACCTTAGTGGAAAGGGTTAGTTGTTTAAGCAAGTGGTC
TCGTGTTTGAGCCCTTGTTAATGCAGCAAATTTTCTTTGGAGAAGTTCCAGTTTTGATACCCGACGTAAC
CTCAAAGGGAAATAGGCTATGGATGCCTTGAAGAATAAAAAACAACCAATTTGATTAGGAGTTACAATGA
TAGAAAGATTTTTTTATTCATTGAGGAAAGATTAGGATCAGAGGAGTCAAGAGGAAGATGAGTTTTTAAA
TCCAACTTCACTAGAAACTAATCCTTTTCCTTAAACAGATTGATTATTACTGGCTGCTAGCTAGAGTGGA
CATTGTAAATTTATATATATATATATATATATATATATATATATATATATATGTTAACGAGCCGTTAACA
AACCGCAAGATAAACTAAGAATAAATCACAAAACCACTACTGGAAATATAAAACAGGGAATCAAGAGACC
ATGGAGCCGCAGGTCCAATACAGAGGAAACAACAAAACAGCAGAGATCGTAGATGGTTTCTCTCTTTAAA
ATACTTGAAATCAATGGTTAACCATAAGCATGACAGACACACACATGCTTCTGCTACTATAAGAACCCAC
AAAGACAAGCCTTTCTCTTCACAACACAAGAAACACAAAGTTCGTTCAACACAAGAAACATAAAGTTCGT
TCAAGACAAGAAAGAAT
Sequence ID: 19
Sequence Length: 3065
Sequence Type: DNA
Organism: Poplar sp.
TTTACAACATATATATGTTATATTCACACGGATTATTTCTTAATTTTTTCATATAAGATATTAAAATATC
AAATATTTTTTTTAATTTTCCTGGTTTGATGCGAGTCAACTTATGTAACCCAGGACCCGATCCCCTGGCC
AGGTCAACCCCAAAGTCGGGTTTGATAACTATAGTTGTCATGAAAATATATTTTATGATTTGTTATGTTT
TTTGAACTATCAAAATACATTTATTAATCTGCATAAATAATTTTTTATAAAAATAAAAATATTTAACCCG
TGATGAAGCGCGGGTGAAATAGCTAGTAAAAACTAAATAATGTTTAATTGTAGGCTAAATTAAAAAGTTG
AGGGAGTCATTTTCCATTCGAACTCGAGCGCGAGGGGCTCTCCCCAAGGAGAGAATAGTCTCTTACTTTA
CGACACTACTTGTGTCTAGATAGTTGATCTTGAAAAACGTCAAAAAAGAAATCTAATTCGTGATTTCTCA
AGATTTCATTTAGGAAGGATGTCCAAAGATGGCGACATGATGTTGTCATTTGCAAGGCAGTAACACCGAC
TTTTCTAGAGCTCATTTTGACTCACAAGAGTCTCGTTACCCATCACTCAGTCCATGATGATTATTTGTTT
TTTTAGTTGAAATTTAATTTAATGTGTTTTTCATGTTATTTTTCATGTGTGTTTTAAATGATTTGAACGT
GTTTATATAAAATAAAAAATAAAATAAAAACATGACGCATTGCATTACCAATCACGCACATAAAATCCTT
CTTCTTTATCCTCTACTATTTATACATTTCATGTTTCTCGACAAAGCTTCATGGGCATGCCTTTATCATT
TTTTTAATAAATTTCAAGACAACAACGGTTATTATATGCTATATATATAAAATCTATAAATTAAAAATTT
TAAGTTTTGATTAAATTAAAATCCTACTCAATTTTCTAATAAACTCTTTTATTTAAGTTCAAAGTTATTT
CTAACTAGATATATTTGTGATTTTTTATAAGTTAAGTATAAAAATAACTAAAATTATTTTACAAAAATTA
TACCAGATGAAACGTAGTTTTACTCCTTTTTTTCCCCTCTCGTTTCCTTCATATTATTTCCCAAATATTC
TCATACACTAAACACATTGTTAGGATCCTTGCAGCTTAGAGTTGCTAATCAAGCCCTTTTAATTAAATCA
ACTTTAAAACACTTCTTCATCATAAATAAATTAATTAATTAAGTGCTCCTGGAGTTTTTAACCATCTTCC
TTGCTAATCTAAGTAGGTATAAATTTGATTTTTTTTCTTTCTTGAAATTATTTTTTTATGTTTTTTAATT
ATTTTAATATAAAAAATTATATTTTAAAAATAAAAAAATATTTTGATATATTTAAAAAAATTATTTAAAA
AAAATCATTACTGCACTTCGAAATGTCATTTTGAAACACGGTGCAATTGACTTTTCTCAATAATCTCTAA
ATCTTTATTTAATAGCGAGATTACTGTCACACCAAAAAGGTCTAAGGCTTTTTGAGCCCAGGTCACTTCC
CTTGCACTCACCTGGCTCTCAAATCTGTCACCAAAGCCCACAAAAGCCCACTTTTGCAGGCTTGTTTCAA
AAGCCCGAAATCAATACGAGAGACTCACATCTCTAAAACCCAACTAATTTAGAGCAGGCCCACCACGAAC
CCAAGTCACTTTCGCGAAGAAAGTTCCAAGTAGAGGCATAGTTGTGGCCCAGGCTCTGAAGTCTGAACTA
GTTTAGGCCTAGATACATCCGAGTGAATTATTATTATTTTTAATAGTATTATTTTTTACAAAATTCAATA
GTCTTAAGTATCAATTCATTAGGTCGTCAGTATAAAAACAAACAGTTCAAAATAAAATTTAATTTAAAAC
AACTCTTAAATTATATATCATAAAATCCATTCCACAACCAAACTAGATTTTGAACCTATTTATTTTTATA
TTTCAAAAATACTTTTTTAAAAAATTAAATCGTTTTACTTCAAATTAAGTTTTTTTTATATTTTAATTTT
TTTGATGTTTTGATATTAAAAAAAAATTATTATTTTAATATATATTTTTTAAAAATATTTTTTTTAAAAA
ATACTACCGTATTATTTATATTTCGATTGATGATTTGGCTTAGAATAAACTTAAAAAATTCCAGGTAGGC
TTGGCAGTTAAAATAATTGTATTTATCATTAAATAAATGATTATTCCATTGCATTTAGAATTTCTCTTGC
AGAACGTGGAACATGCCTATTGGCTACCACAGAGTTGTCAAAGCAATGACCTTGTACGAAAAGATTTTGT
TCCTTCTTGGTCATAAATTATAAATCTGGCTCACTAATAATTAATAAAATCATTTTTTATATTTAATATC
AAACAATATATTTTTTATTGGGTTTATGATTTTTTTCTGTTTGATAACAAAATATTTAAATTTTTTTTAA
TCTTTTAAATTCATATATTAAAATTAATATTTACATTTAGATGATATATTTTTATTTTTTTCTAAAATAT
TTAGATTAGGTTATAATGAAATTAGGCTGGGAGAGATTATATAAAAAAAATATAAGAATTTAACTATTGA
ATATTTGGTCATAAGAATTTTAGAATTGTAATACTAAGAAAAATAATTCTAAACTATGATTGAATTCTAT
GAATGACTGTTAATTAAAGTATGATAAATAATATAAAATAAATGATAATATTAATACATCAATACTTATT
TCATTATTATTAAAGTGTTATTATTGATATTATCATTTATTTTTTGTTCAGTAATAAAAAAATATTATTC
AATCAGTTAAACAATCTTATATCAATATAATATATTCATAAATTATCTTAATAAATTATTTTTATAAAAA
AACCAAATAACTAGTTTTTTTTTTTTTTTTTAATATGATGGCCTCTTATTAAAAAAAAAGAAAAAAAAGA
AAGAAAGGGGGAACTCCTTGGCCCCTATTTATCTATAAAAAACATCCACACCTTCTTCCACCGCCACTCT
CTTAGCTCTATAATTGACTTCCACTGCATCCATCCATCCATCCATCCATCCATCC
Sequence ID: 20
Sequence Length: 3000
Sequence Type: DNA
Organism: Poplar sp.
CTAATGGGTTAAAACCAGAAATCAACTAACATAAGTTCACTAATGAAATAAACTTTTAAACAATGTCTAA
TGGGTCAGAACAAACCTAAATTAAAGATAATTATTCAACATCAAATAAATAAAATAAGATAATAAAAACA
AACTCTTTATGGTAGAATTCCTCCATGTATCTGAAATCTCCAAGCTCTCCAGAGTGGTTTTTTTGCCGAA
ACGTTTAGTGGAGGAATTCAACTAGAAAACTTTATTTTGATTACTAGAATTCACTTTCCTTGTATTGCCT
CCTCTTAGTCTCTTTGATGTGCTAGAAATACCTGCAAAATAAGAAGAAAAAAAATCAAGGAGAAAGATCC
CCTTATTAGATGTCGTCACATCAACTTCATGTAGGACTTGGAATCCTCGCAAAGTATGGACTTCAATTCT
TCAATGAATTCAAACCACATTAGGAACCCTTGTAGCCCAAGACCATGCACAAGTTTAAGGCCTAAATAAA
CCTAATTTAATATCCATTTGTTGTCCAAAACTAACCCATTACAGGTCTGAATCGAATGGGCTAAAAATAG
CCAAAAAAGAACACTTTGTCGATAAATCTGTCAAAACAGTAACAACAACTTTAAATAATTTATTCCCGAA
CTTTGATTGACCTGAGATTTTATCACACTATAGTTTCATATGTTTAATACCTCATTATAAATTTTAAGCT
CGATTTCAATATACAGTTTGAAAAATATATTTGATTTTGCAAAACTGATCTATTTAAAAGCAAAATTCAT
ACCCAACTTGGTTTCTACCTTAAAAAAAAAAGAAAAAAAGATTAATATGTGAATTACACGCCTAGTAGTG
TAGTGATTGCAGAAGTCCAAAAGCAGTCACATACCTAAAACCCAACTGAGATGTAAGGCAGGCCCACCAC
AAACCCAAGTCATTTTCGATGGAGAGAGCACAGAGGAGTTGTCACCTTCGTTCTGCTTTGAAGGGCCTAG
TAGTTGTTGTTTAGAATTTGCATTTCTCCTCTGATCACTAAAGTTCAAACTTCAGAGAGTTGAATATTCT
AAAACGAAAAATCTGTGCTAACATCTTGTCATTATCGGCAGACACAGGCAGGCAGGCTAGATAGGAGTTG
TTTTGCATTTTTAATTTCACTTTGGTACACTTTCGTGCAGCGTTTCGTTTTGCATTTGAGCCATCACCAC
CTCGAAAGTCAATAGTCAAAAGTCAGTCCAGGGTGGCCTAACAACAGTTCAATAATTGTATTTATCCTTT
GGTGTTTTTTTTAATAAAAATTTAATTTTTTATATATTTTTGAATTTTTTTAATATGTTAATATTAAAAA
TTATTTTTAAAAAGTACTAAAAATATTATTTTAATATATTTTCGAGTGAAAAGTATTTTGAAAACCAATC
ACTACTACATTCTCAAATACCTCGTTAATCATTCATTTTCAAACCTCAAATGTTTTCTTTAGTATTCTAT
TAATTATATTATTTAAGGTACAATATTATCTTTTTCTTTTATATTTAATTGTTTCACACATGTATTTCAT
TACATTTATAATTTTCTTTGTTCGTAGAACATGGCGGATAAACCCAAATTTAAGAGCCTATAAAACCTAA
TATTTTTCTCGGCTCATCGAAGTGAATGACCCATTTCCTTTGGGCATATCTACATTTCCTCTAGTGTGAT
AGCTTCATTCTCCATGACAGCTTGAGACGGTCGACCTCAAAAACATAATAAAACCCTTGTTTTATGGCCA
AATATATAGCACCATGGCTAAGTATGAATTCTTCTATAATTTCACCTTACCCTCCTAGAAAATTTTCTAA
GTACGAATTCCTCCATAGTTTACCTCTATATACTTAGAAAATTGTTTTTCAAACATGGGACATACCCTTA
CACACTCATCGTACTAAGTATATTAACCGGGGTTTTCACCCCGCTTCTCAAAAAATATTTTTTGACAAAC
TCGTCGTATTGACCATATTTACAAGCCTTTGCGTATAAATATCACAACGATTTAGGGGGGCTATTGATAA
ATTCAAATTTAAGGGTCTAAACCCCCCATAAAACTCAATCTCATTTGGGCTCAGTCAAGGGAAGGACCAA
CCTCTTTTGAGCACAACTACATTTTGGATCATACTCTCTCTACATACATTAGGTGTATAAAAGTCTTCAA
AGGACCCACCATATTTCTATCGATATTAATGCATATATTAGGTATTTTTCTCTCATTAATATACATATGT
GAGGGATATTTTTTCTTATTAATACATATATCAGTGATATTTCTCATCAACATCAATACATATGTCATGA
ATATTTTTTCTCTTTAATATTATCAGGAAAATATTACACTTTAGTCATTCCTCTGCATAAAAATACTTAT
GACTATAAATAGACCATAAATTCTTTAAACAAAAAGTTTGGAATTTTCATTCTCTACTACATATTTATTT
TCATATTGTCTCTGTAATATTATTGTATTTTCTCTTAAAAAATCACTGATTTAATCATTGAAGAGTACCT
TAATTCACTACAAATTATCTTTTACAAGTATTAAATATCTTGACTAGAGATGACAAATCAAATTACTAAA
ATCAAGAATTTAACTAATTATCTAAAATATAAACCTTATCCCAAACTAGTAGGAGGTTTGAAGACGTCAG
CTGGATATTAATTGATCACATTCCTTCTCCATCGCAATTTATTAAATTATATTAACACATAAAAACTGTG
CTATATATTGTGAATGGGTACGTACGTACGTCGTTTTGGTTTAATATATATTATAATTATAAGTATAACT
ATAGTAATTAGAAAAAAATAGAAGAATTTAACTTTACAATATTTAGTTACAAGCATTTTATTTTATTTTA
TTTTATTTTATTTTTTTAAAATCCCAGTTACTTATCTTATATAAATCCCAGTCCTCTTTGAAGTTCCCAC
CATTTGCAATCTTCAATTCTTTCTCTCGTTGGTTTCTTCTGTTGACTTCACTCTCCATCC
Sequence ID: 21
Sequence Length: 3022
Sequence Type: DNA
Organism: Poplar sp.
GCTTGCACACACAGCCACATGCTAGCGAAGAAGGAACACAACTGCTTTTAACTTAAAAGAAATTTAGAAA
TGTATAGTTTGCAAAGGAAAATGCTCGATGGCTCCTTTGGCTTTGGCCTTTATGATTTTTTGAATTTTCA
ATATTGTTGGAAAAAGTTGTGGTTTGGAACGTATTGATTAGGGTATCACAGGCTAGCTTTCATTTTTCAT
TCTAATTAAGGAATTAATTATTTTCTTTAACCAAGTCTTGTATAGGCAATCCATGTTAGAACTAGTATAT
TAATTCCCAAGAAATTTTTTATTTTATTTTAAAAATGATTATATTATATGGAAATAAGCAGCAAAAGTCA
TTTTTGGAGTCCACCATAGAATGGTTTTTGGTTGGAGCTTAAATCATTCACAACATGTGCTGGATCGGCA
CGCAGCTAGCTAGCTAGCTTCTGGTGTGCTTATGCATGGATGTGCTGATCTTGATATCTTGTGGAGCAGC
ATCATCAGCAGCAGCTTCAGCACTCAAAGGGGTGCTTGCCACAGCTAAAAAGTTGTACTTCAGGAAACAA
AAAATGAGCCTCCATTTTTCACAAATCTCAACCTTTCTTTGTAAACTTTCGATCAATGTTGTTTTGTGTG
AGCATAATCACTCCATTAACAAGGTCACCGCATTATCGTACGCAGAATTAACCAGAATAATTCTTCATAA
AACCAACAAAGATTGGTTGTAGTAATCTTGAAAAACTAATTTCACCATCATTTATAAAATGTGTCAAGTA
ATAACTCAAGCTTAGAACAATGGACTTTTTCAAGAAACTAATAAAGCTCTGCAGGACCAATCAACATATT
CTATGACCATTCTGTCAATTTTTTTTTTAATTTTTATCACAGGTAATTTTTGAATTTTTTTGCCCCTTTC
TAACTGTTTCATTATATATATATACTAGCTTTGATACCCGCGCGATGCCGCGGGTTATTTTTTTTGCATA
AAAAATATTAAAAAAAATAAAACTTTAAAAATCTTGGGTTTTTCTATAAAGTTATACCCAAGAATTTTGG
ATTTTGCTGCAACGTCTGACCTAAGAGTAATATTTATAATATTAATAATAAAATTAAACTTGTATGACCC
AAGTTTAAGTGAGTCTGGCTGTAACACCGGACCCAAGAATACTGGATGTGGGTCTGGCTATAAGGTCGTG
TCATAAAAGTGTGATAACTAAATAGATTAATTAAAAAAAACAAAGAAAAAAAATCAACAGGAAGGAAAAA
ACTAATGAAGAAAAAAAAAACATTGAATTAATTGGGTCAACCTTTTAAACCAGGTTATCCCGTTTTGCGT
CATAAAAGTTTGATAACTAAATAGAAAAAAAAATGATGGGTTTACCTAGAATTAACCGGGTTAACCCATC
AAACCAAGTTAACCCGTCAAGCCCAGGATACGTGTCATAAAAATATGAAAGTCTGATAATTAAATAGAAA
TAAAATTAACTTTAACAAACTAAACTAAATGAAAAAAATAACTCGTCAAATCAGGTTAACCCATCAAACC
CGAGATTCGTATCATGAAAATCTGATAACTAAATAAAAAAAATTAACATTAACAAACTAAATCAAACAAA
AAAAATTCATTAAAAAAATAAAAAGAAAAAACAAAAGAAAAAAACAGTTATATAATATAATACAATATAA
TAATTATTATAATTATAATTATAATGAAAAAAAGTGGGGAAAATTTTAAAAAATGAAAAAAAAAGTGGGG
AAAGCTAAAGTTAAAACTAAATTCTCAACCAACTCAATATTTTAAAAAAATTAAACAAAGATAATTTAAA
AAAAAAACATGTGGGGGAAACACTGTAGCCAAACAAAAACCATGTAAGGGAAACACTGTAGTAATTCATA
GTATTTTTTTTTATAAAAAAGCTAGAAAACTAATCTCTCAACCAGCTCAATATATAAAAAAAACCGACAA
AGGTTATCTAAAAAAAAAGAGTCAATTTTGGGTAAAAGAAATGAAAAAAAAATGTACAAAAAAGGAAACA
AAAGCGAAAAAAAAACCACGTGGGGAAAGCTACAGTGTTTTAAAGAAAAAGACAAAAAAACTAAATTTTC
AACCAGCTCAATAGTAAAAAAATAAAATCAACAAAAATAATTCTGAAAAAAATAAAAATAAAAAAAAATA
AAAAAAAAACTTATGGGGAAAGCTAAAGCTAGATTATCAGCCAGCTCAATATTGAAAAAATAAATTCGAT
AAAGATAATTTTTTAAAAAAATATATGAGGAAACACTGCAGTAAAACAAAAACTATGTAGGGGAAACACT
GTAGCAATCCATATTTTTTTTTTTAAAAAAACTACAAAGCTAAATTCTCAGTCAACTTAATATAAAAAAA
TAAAATCTACAAAGACCATTTTGAAAAAAAAAAGAATAAAGAAATCATAAAAAAAAAATAATATATGAGA
ATATTATAGTAATCTATAATGTTTTAAAGAAAAAAGCTAGTTAAATTTTCAACCAGCTCAATATTTAAAA
AAATTAGCAAAGATAATTTTGAAAAAAAAAATTAAAAAAAAAACAAAAAAAAGAAGAAGAAATGAATTTT
GGGTAAAAAAAAAAGGAAAAAGAACGAAAAAAAAATATGAGCAGTTTATAATTATAATATAATATAATAT
AATAATAATTATTATAATATTCTTATTATAGCGTGGGGAATGGGGAAAGTGGGGAAAGTTAAAAGGCGTG
GGGAAAGCTACAGTGTTTTCCCCACGCCTTTTAGAGTATTACTTAATATATATATATATATATATATATA
TATATATATTCACCAACTACCAAACTAGAAAAAAGAGTCCCCACTTCCCAGTACCTACCAACCTACCTAC
CCATCTTTGTTTCCTGCCATTAAATAAACAGAGAGAAACGCAAACTCTCATTGATTCTACCATCTCGTTT
GCGAAACCTTCAAGATTCTCTTCTCTGCCACCATATCCCTTTGAAAAAGCAATCAAAATGTCATCTTCGA
TCAAGCAACCCA
Sequence ID: 22
Sequence Length: 2846
Sequence Type: DNA
Organism: Poplar sp.
CAGAATCAAGAAAACAAAAGGGAATTAAAAAAGAGAGCCCCCCTCCCCAATTCTTGATGTAGTATAAAGAATTC
ACTTAAGGAGAAGTAGTCGTGCCACATTTCATATATGGGGGTAGTGTCTGTCTCCCTTGTGCACTCCATTGTCA
TGTCCGCTTGAAGTCATGATGGACCTCATGGCTCTTCTTACTTGTGTCAGCCGATCACTTTATGTTATCCCCTC
AATTGTATTGTCATTCCATTTCCTCGGGTGTCATCAATGGTTAGGCTCTTTGTCCTGACTATGTCTGTGCTGGA
AGCCCAGGGGTCTGCGTGTAGGTGTATGAGTGAAGGCCGAGGATTGGTGCCTTTTGAAATTCAAAGTACTTGCC
ATCTATTTGCCACGTCTCTCATTAGTGGTGGACGTGTTGAGGGGCAATAGGGATGAATCATCCATTCAAGTAAA
CAATGCCAACATTTTTTGCTATGTTGGTAGATCTATACATGGCAAGGAGTTGGTCTTTTTCTTTATTTTCACCA
TTTCCATTCACTTTGTCTACCTTTTTCTCTCTCTAAATGATTTTCTGCGAGGCAATACCACATCCGACCACCAC
TAAAACATGCTCAAATTCACGACAAAACCCCATTTCTCGATTCTCCCATAGTTGCTATAGAGTTTTAGGACCTT
TATTATGCCTTTGAGTTAGCATTTCTAACGGGTCCATAGCGTTCTTATATGTTGTTCTCGTTTATTTCCACCAA
AATCCAATCATCAGGATTTTACAAGGTTAGATACTTTTTTTCAACTTGAAGATTTTTCTAATTATAGCAAACCT
AAGTAGGATAGAATCTTGGTCTGTACTATTCATAGTGGAGATATCTCTGAAGATCAGTTAGTAAAAGGGGCTAG
GATACACCATACACCTTACGAGGGGTGCAACTTCTCATGCATGTTCAGATACGGACACGACACTTCAAATATTC
ATAGTGTATGCAGAGGCATGATAATACATTGAAAATTATTCAAAATAAATTAAAATTCATTCGAGTTTCAAAAA
CGGCATTCGAGAGCTGCTGGAGGTGGTCGGAAACTGTTGGAATTAGAACTGGGCAGCGATTGTGCGTGGTTACA
TGTGCTATGTTTACATGGATATTGGCGTGAAATGGAGCAAGGAAGAGGGATGCGTGACTCACACAATGTCTTCG
AAACCCATGAAAACGGATATAAATGAAAGCTTGGTTTAAGTGGAGATGTGTTGGCTACTTTGATCGGGAAAGGG
GAGTTGGGATTGGTGGTCGCAACCGTGCTTTGATTTTTCATTTAGTTGAGGAAGATCAGGTGCTGTGGCTACTC
TTTTGGCACGGTGCTGCTGTGTTTTTTATTCTTTTTTCGAAGATGGCTATGTGAATGGGCTGATTGTTGCAATA
GCAGGTTGTTTGGTTGGCTGGTGAATGATTGGCGAGGATGGTTGTTAAATTTTAGTCATGATTTTTTTAAAATT
GTTTTTTATTTAAAAATATATTAAAATAATTTTTTTATTTAAAATTTATTAAAATAATATTACAACGGATATTA
TTTCTCCAGTACTTATTTATATATCTTTCACTATATTATTTGATTCTCTATAATTCTTACTTATCATTTCTTTT
TATAATATTTTTTATTTTTTATTTTTCAATCTCATTATTTAATATGATATTTTATTTCATTTTATCGGTTAAGT
ATTACGTTGATAATAAATTAATTTTTATATTTTTTTATCGAATGATAAAAGTTCCCCCCACCCCTCCTTTATTT
ATGTTTTGGCTTTTTTATCAAATTTTATTTTTTAATATGAAGATTTTAATTCCTTTAAGTTTCTTTATTTTTTT
TTTAATTGTATCCCTTAATATGAGCTTTTTATTTAATTTTATCATTTAATATTGTATTGATTTTTTTTTCTGAA
CTTACTCAATGAATAAGGTCATGTCAAGATAATTTTCACATAATTTAATTTAAGAATCGGGTTTAAATAAATAC
ACGGGTTGGGATTTTTTAAATTTAAATAGTTGTAACAAGTTTTATGATAATATCAAAAAAAAAATTTCAACAAT
TTAGATGTTATTTTTTTATATTCAAAATTTTTTTTAAAGCAACACGCTCCGATAACACGGTAAAACATCATTGA
ATTGAATTAATAAAAGTGATGGAATATCTAATTTTGAATTATAATTCAGAATAAAACATGCCATGAGCTGTGTC
AGATAATCCTCAGCCAACTAGCCTGAGCATGCTCTCTAACATAAAGTCCTCAACCCCCAGAACTCTGTCTTTAT
CAAAATAAATAATTTGCCTGTATTGTGCAAGTTGAACACTTATCCACATCCTTGATTTAGACTACGCAAAAAAA
GTAAAATTATTTCCCCTCATTAGAACAATAATTTATTCTCATCTTGTAGCCTCCAAATAATAGCAAAAATTAGC
CAGAGTACTGATTAGACATGCTAATTTTGGTTGAATTAATCCCTTCAGACTTTTCTCAATATATAGCAATCCAT
GGTTGTTCATGTTAATTCTAGCGTGCTCGTTGGCTTTTTTTCAGATTACACCAAGTTTACATCTCTTTTTAGCT
AGCTACCCAACTACTATAAATATATGAATGTAAACACATGCATGCTGTAGTATCAAACATACCAAGAGTTATCA
AAACATGGCAATGCTCTTGCAAGCAAGAAGAATATTGACACAAACCCTGCCGAGATGCAACTCAAACTCATCTC
AAGCCTTCGTTAAGTTACTAGGATCGGCGTCTCGGGGCTTGCACGATAATGTGCCGGGAAGTAAGTTGTTCGAT
CCGAATGCGACGTCAGGGAAAGTCATCACTTGCA
Sequence ID: 23
Sequence Length: 3025
Sequence Type: DNA
Organism: Poplar sp.
TAACATACTTTATTTAATAAAAAAATTTCTTTTATAGAATTCACTTATCTTAATGTTAATTTGCATAAAT
ATAGAGTCTTTGTGTATATATTTTATCTAGGTCCAACATCAATATTTACTTAAACATTCCTTTTTTTTTA
GTTTCTAATTATTTTTAAAGTTATTAATACGAGTTAATTCACCCAACTTAAGTCTTAAGCCACGTCATGG
ACCAACGAGGTATTGTAACTATCGTTTTACCTATCTCTAGTTGGCCATCTCGTGCCTCTATTGATGGTGT
ACAAGTCCCCCTTCTGTTCACATTTAATGCTAACTACTATCAAAATGTCGCGTATATATATATCATGTGC
ATGCAGAAACAAACTAGCTATAAATATATATATAGTTTCCTCACAGGGAGACAGGCCCCTTGCTTCATAA
ATATTGGCTTCCAATCATGATCTAATTCTAAATCAAAACTGTCTGAATGCAGACGCCAGGCCAATTCAAC
TTTGTTGACCCACTGACCATGTGTCAATATTTAATTTTCCCATTGTTTCAGGGAAAGAATGGAAAGCAAC
ATCTTCTTTCTTCGACTACATTAATTTCCCCCCTCTTATTTAACCCCTTTTCCTTCTATTATTTTTGTAT
TTTCTGTTATATTAAGCATGGTTTAAACTTGATTCAGTATTAACTTGGGATAAGACCTGCGTCAGAGATT
CAACTCAATTTTTTTAAAAAAATTATATATTATTTTGATTTTAAAAAAAAATTATTAATGGATTTTCAAC
CTTATTTTATCTGGAGCGTCCGAATCCCAGGTTAATTCTGATTTTTAATTAGATTAAGTCATGTTAACTT
ATTTTTTTATATTTTTTTTTAAATCCAAACCAGTTTAGGTTCTAGATCAAAAAATCAAGTCATTACAGGT
TTAAAACTATAATATTAAGATCATGTGAATTTTTTTCTCTTAAGATCTTGTGAATTTTTTTTCTCTTAAA
ATTTTCAAGGGTTGCATGCCTGGATTCATTTTCAATGATATTAGTTCATTCATTCATATATATATATATA
TATGATTAGGTTCATAGTCAGAGATGTGCTCCTTCGTTTAACAAATAAAATTCCGAACTATAAGGAATAG
TTTTTTATATAAATTTTTTAATTTTTCTTAATTTGAGTAGACCAAAAATATAGATTCACATCATGTTTGT
TATTTCCATAGCTTTTTCACAAAGAGTAATATATTTCTAGCTATCTGCAAATTTTCTCTTCGACAAAACA
TCTAATACTATTTGAGTTTTTTTTTCCATTGCAATCCAAACTCACACCTCTAAAATTAATCCAAAAATGC
AATTTAACTCTATCTTTCTGTCTATCTATATGTATTTTTTTTTATGAGTTTTCACATTCCGGATTAAGCC
AATCTCTTCAATTTAATTAAAAAAAATCTATGTACTTCAAAATTACAAAATTCATAAAAACTTTACTTCT
CACGTCCTATATTAAAAGGATGTTTGGTTTAGAGATGTGGTGATTTTTTTTAAATATATAACTCTTTTAA
AGCACAAACTACATCTTCAAAAACTAAAAATATATATTTTTTGTGTGGATCCCAAAATAAATACTAGAAT
CACCTTCAAATTAAACACCCTCTAAGATTTGAACTTCGTGATTCCCCCGGCTTAACCATGATCTCTTTAT
CTTTATATTTATATATATAAACTTGTCAAAGTCATAGCAAATTAATCATTCAAGCAAGATAAATATTGCT
ACTAATAAATTCAAACATTAATAGAGGGGATGGATTTTTTTTTGTTTAATGTGGGAGCTTTTGTAAGAGA
ATTAAAAGAGGGACTAACAATAAAAATGACAAATGACAAATGAGCAAACATTAAACTAGAATCATTTTCT
ATTCGCTATCGATGAGCGAGGTTTGATCGAGGTTGCTGCAAATCTTTCGAAGATTATGACTTTTTCAAGG
CTGTTATGGGCTGGTAGGCGCCCTAAATTGAACAGCTTCCAGTTTACTTATATTTATCCCATTAATAAAC
AAAGACTTCGCTTCCATATATATTTCATGACTCTGATCAGCTCAGTCATTATATTTTAATGGTGTGAGAG
CTTCATTATTGGTGGAGCATAAACCATCAACGCATAAGAAACCTTGCTTACTCATGAATCGGTGAAATGC
GGCATTAGGAGTAAATTAATAACGTTAGAATTTCCGGTTTTTTGTTTATTCAACAAGAGGTCTAATGGAT
TTGAATTACATATTTAATAAGGATAGAATTTCTAAGTTTGAATCATAATTCCAAAATAAGAGTGCCCCAA
CAAGGATAAAGATATCTTGTCCATAATTCAGAGTAAAGGAAGCATCCAAATTTAGCCAGCATCCATGTTT
TTTAACCTAAGCTGTCTCTTTTTGAAGAGATTTTTACCTTTGATTGACTATATATATACACACATTTATA
TATGCAAGTCAGATGTTCTAGTCCTCAACCCAAATTTCAGTATTCTTTTTTTCTCAAAATATATAATTTT
CCCGCATTTCTCAAGTTGAAACTCTTGTCCTCATCCTCGATTTAGACTACGCAAAAAAAAAAGTAAAAGC
ATTGCCCCTCATTAGAACAATAATTTATCCTCGGCCAGAGTGCTGACTAGACACGCTAATTTTGGTTGAA
TTACTCGCTTCAGACTTTTCTCAATAGTAATCCATGGTTGTTCAAGTCAATTCTAGCGTGCTCGTTTACT
TTTTTTAAGATCATACGACGTTTACATCTCTTTTTAGCTAGCTACCCAAATACTATAAATATATGCATGT
AAACACATGCATGCTGTAGTATCAAACATACCAAGAGTTATCAAAACATGGCAATGCTTTTGCAAGCAAG
AAGAATATTGACACAAACCCTGCCTAGAGGCAACTCAAACTCATCTCAAGCCTTCGTTAAGTTACTAGGA
TCAGCAACGCGGGGCTTGCACGATAATGTCCCGAGAAGTAAGTTGTTCGATCCGAATGCGACGTCAGGGA
AAGTCATCACTTGCA
Sequence ID: 24
Sequence Length: 3161
Sequence Type: DNA
Organism: Poplar sp.
AATACTGGGTTAAGTTGGGCATGGCCCTGTTTCCTAAATTTTTAAAACAGGGCATTATGTATTGGTCCAA
AAATACATCCTCCTTCCTTTCTTTTTCTTTTTTTCTATTTTTTCCTTCTCTTTTCTTCAGGTTGTTTTTT
CTCCTAAACACTCTTCTTCTTCTTCTTTTTTTCACTCTGGCACTATCACGGCGATTCCATACATAAATAT
ACTGGGATCTAAAACAGGTGACCTGGATCGTAAAACGTAGGGGTTTTCGACTAATCTAGCAACTTTGTTT
CAGTATTATTTAAAGATACATGATTCAATCTTGTTGACACTATATAATTTATTAACAGCAGCTGCTTTTA
TGATGAAAGTGCTTTTTATTTAAAATAATGTTTTTTTTTATTTTTAAAAATTATTTTTAATATCAACGCA
TCAAAATTATATAAAAACACCAAAAATATATTAATTTAAAATAAAAAAAAATAAAATTTTTTCGAAAAAA
AATTTAAAATGCAAAATCAAACAAGATCAAAATAATGATACAGCAGATTTGAGAAGGGGCATCTGCTTAA
AGAAAGAAAAAAGAACCATGCATTTGACTATAGTCTAGATAGAAATCATGCCAATACACCAATGGTTTCA
GCTTGTACTAATGATAATCAGAGGGCAAGACTAAATGCAATTATACATGTTCTTTATAGAAAATACATTT
AAATAAATAATAATTAAAAGAAAAAAAATTAAATCTGACATAAAATAAATTGAACTAAAATATTAAGGGA
TATAATTGAAAAAATAATTCAATTAATAAAATAATAAAAAATATTGAGCGATGTAATTGAGATGATAATT
TAATCAAGATAATAATTAAAAAAAACTTAACAATAAAAAGAATGAGAACTAAATTTGAAAGATAAAAAAA
AAAATCAAATGAAAGTTAAATTAAAAAAAAAATTCAACTCTATGAATTATTTAAAATAAAAAATAGCAAT
AAAAAGAATATAAATCAAATATGAAAGAAAAAAAATCAAGGGACGACTATGAAAATGGATAGGGAAAGGC
TTTGAAACCGAAGGAGAGAAAAAAAAAGAAAAGAAAAAAAAAGTCATTGGAGCAGAACCTGATATTTTTT
TTGTACAAGCGCCGCTCATTAATGGAGGAGACACTATAAGGAATAAAACGCTATTGTAGAAGTTGTATTT
TGACCTTTGAAGGATGCTTCATGCATCAGACAAAGATCGTAGAAGTATTCATGCACTTTCAGGTGAACGT
GACGCACAACTCATTTTTAGAATTAATTAATATGTTATATACATGTCTAAATAATAAACCATCAATAATC
AAACTTGATAATAACAAAAAAAAATCAAATGGAAAAGATATTAAAACCTCTTGACCCAAAACGAAGACAA
GTTTAATCCAAGAACATTTCGGTAATTGTATTGTGTATTTGTAATGGAAAAACAATAACACCCTTGCTCG
CGAACTTACCTTTTCTTTTGCCTCTAAGTGTATATAAGATATTTTATTGTGCACAAAACAATAAAAAAAC
CTCTTTACCCTTTCTCTCAAGGCCAATTGTGTTACATTTACAGATGAAAAACCGTAATTTTATTGTTCCA
ATCCATAATGACTTTGGTTTCTTTTAATGTCATACAGCAGGCCCAAAGGCCTAGTAAATGCTTTCACATA
CAAAGAAGAAACTACATGTCACTAGGTGGGGCACTGCCCCCTATCCCGCACCCTCAATATAACTGTATAT
TTTGATCTAGTGGCCAATTTAAACCTAAGCCTAGCTTGAGCTCGACTGAGAGATTTATTGATGTTTCATT
TAGGTAACGACACTCTAATAATGGTTCAAGCAAGCCAAACAACCATTCAAGCGTTGAATAGCTCATTCTA
GAGATGGTTTTGAAAGAGTCAAGATGAAAAGAACCTTATTCCAACTCATTAAGTTGATTTGTGAGTTCAT
GGTTTTCAACTCAGTATCTAATTTTTATAGTTGAGTTCTCTCCAGTTTCTTGAATGTCAAGATTTGAGAG
CATTTAAAGTGAACTAATGATTTCTTGAGAAGAGAGAAAATAATAAATAATTTGTAAAAAAATTCCATGG
GAATTGGAATTACGAGAATTAGAGACAAATGAAAGGGGAGTTGACGCAGGGAAAAAAAAGAGAATACGAA
GGAGAAGCTTAAAGACAAGATAAGAAAGCAAAGCAATATAAGTTTTTGATGTTCATCACATTATTTCTGA
AGAAAGCAAAGCAAAGCAATATTATAGGTTTTTTAGTCATTAACTCTGATCCATTACATGAAAGATAGTT
AAAATAAGATTTAACCTTGAATTAAATGAGTTTATTCTAATTGGTCTAAAAGAAAATTTACGGTATTGTT
TAGTTGTTTATATGAAATAATAAATACAAGGTTAAATTGATCATTTTAATTTAAAACTTTTGATTGATGA
AGAATATTTTCTATAATTCTTAGAATAATAAAAGCAAAGTGTAAAATTAAATAAATTATAGGATAATTAG
GATAATTTTCTTTTTAAAAAATATTTTTACATAAAATATTTATATTGTGCTATAAATCACAAACTTACTT
TTGCTTAAAATTTTATAAAAGAAAAAGAATCACAAGCATCATAACTTTTTTTTTTGTTACTATTAATATA
TATATATATATATATATACATTTAGTTTATTACTATTAATATTGGTTTGTCAGGTTATGTGATTGAACGT
GTCAAATTAATGAGGTAAAATCGTGGCTGAGTCCAGTTCGATTGCTATGCCGTGGTCCCAGAACAACATT
GGCCTCAGATTCTTAAATTGGGTGGGGATCCACTAGTGACTGTGAGGAGGCCACGTAACCCACTCTTGAC
CGTGTGAAGACAATCGAAGCCACGTGATCCAATCAGCCTTTTCATTAGATATAGATGACGTGGAAGCAAA
ATGCCCGCTCCTCTCTTGCCCCATCGGCTATATAACAGCCTACCAAACCACCGTCAGATTCATTAAATAT
TCATCATTACAAAGCAAAAAGCTCCAGCTTCAACTCACTGGTAGTGATATTTCTTACTGAACGCTGCCTC
TAGCATTCGAATAGCTTTCACAGTTTAGATATTGATTAAGTTTCTTAATCACAGATCGAGTGATAAACAT
TGTTCTAAGTG
Sequence ID: 25
Sequence Length: 3000
Sequence Type: DNA
Organism: Poplar sp.
CTTTCTAATAAATTAAGCAATTCTTGTCGGTGCATGATGCCACCTCAGTCATGTCCTATCCAACGGCGAT
GCAAACTTCGCTGTCCCGCACTTTTTCATAGGACGAGGTGAAGTTTAGCTATATATCTTTTTTTTTTTTA
ATTTAAATTGTTAATTCTTTATATTTTTATATTCTTTTAATTTTATATTTTTATATTATTTTGATATATT
ACATCAAGAATAAATTTTAAAAAAATAATTTTTAAAATTTACTTAACCACGCAATACATAAAAAATAATA
GAACCCACCAACCTAAGAATACTTGTCAATGCATAGAAGTACACCTGCTAGTTCTTAAAACCAACAAAAG
GAAGCAAAGTAGATCTCTGAGTCAAAAACCAGAGGAAACCATAGAAACACATAATAATAATAATAATAAT
AATAATAATAAAATTAATTTAACTTGGTGTAATAATAAAATTAATTTAATTACAAAGAGTGTAACTCAAC
TAGTCATGTTCTAAATTTATTCTCTAGAGATTACTAGTTTGAGTTTTACAAATTTTAAGGCCACTGAAGA
TTTATATAGTCATTAATTTCAGAATATATAAGATTAGTTGAGTTACGTATAAATTGATTAAAAAATCATA
TTAATAAAAATAAAAAAATTAATTTAAAGGTTTAAGAAATCAAATTAAGAGAAAAGAGTGGTGTTTTATT
TTTCATCGTGCCCTCTCTCAACAGACAAGTAGAATGATGAGAGAGAGAGGGTAAAGAAATGGATTTATGA
GAACATTGACCACAGGGAAAGAGAGAAGCGGTTTTGTGAAAGGAACAATGAAACCACAGGAAGGTAAAGC
GGTAATGATATATTTCACGAATACTAAAACTAGAACAACAAGTTTTTTAATCAAATTAAACCACGAGTGC
AAGGCCGTCTTCTCTGTGTATAAAAGGGTCCTTCTTCTTTCTCATTTCCCATTCTCATCTGCAAACTTCT
CCTTTGCAATCTTTCTTTCTTGCGTTCTGTGTGTTCGTTGTGATTTGTGTTCATTCTTCTTGTCTATTAG
CTTGTCCCCCCGTCCGACTGCTTTCTGTATTTATTCTGGCATTAAGCTTAAGGTAAAGATCCCTCAACTA
TCCCAAGCAATTTATTCTGTTTTTATGTGATCTTGAGGGATCTTCCTCTTGGATGCGCTTTTTATTTTTT
CTTCCTCCTTCTTCCTGCTCCTTCTTACCTTGTATCTGATCCCCCAGACGAAAATGTTTTTTGTTTTTTT
AATTAGCTCAACAAATCAAAAACATTCAGCGCGAAAGAAATCTGATACAGTTTTAATCTGTTGTATTTTA
AAAATCATTACAGTTCATGCATGCTGATACTTTACCATGTCATGAAATTAAATCCCAGCATCCTTTTCCA
TAGCCAAAGAAGGATCAGCAGCATGCTGATAGTTTACCATGTCATGAAATTAAATCCCAGCATCCTTTTC
GCTTGCTTATACAAGGTCTTCGCTTGCTTATCAAGGCCACTGAAACATCATCATCGTCATAACTATGATA
GAACCCGCCTACTGCCGGCATTGAAAACATCATCACTAGTGTCTCTACATTAAAAAACACCCACTGTCTA
ATTTCCTATTTTTTTACTCTTAAAATGTCTTTCGGCTTGAGCTCCTCGGGCTCCACGGATGGCAACTGCT
GTATTATATATATATATATATATATTTCCCTGTTGGCTACATAGACCTGTTAATACCGTATAAATAGATA
ATATTAATATATAGAATTCATGTATCTTTCCGAGATTAAGCGATGCCGTATAAATAATATTAATATCTTT
GAATCAGTATGTATATTAATTAAAATTAATTTTTTTCAAAGTAATTTTAAGAGCGCATTTTCAACATCCA
TTTAGTTTTTTTTTAATAATAAATCTCTCTTTGCATTAATCCTAACGTTTGAACTTAGTAAATTAAAAAA
AGGAAAATACCTTTTTCACCAATATAGAATCAATGAACAGCACTAGTTTGCTTGAAATAAAAATAAAAAT
AAAATCTAATAAGACATTTCGAAATCATCCTTATCCGCAAATCACTACATTAGTATAGTATCTTGAAAGA
TAAGCAAGGATCATGCAAGTTTATAATAATTAAACTTAAAACGTACTATGACGTGTGCATCATTCATTCA
TTCTGCATGAAACTCTCCACAAAGTCTAGCCTTTGCATCATTCATTCTACTTCATTTTATTTTTTCCTCT
AATGGTTTCGATTGATTTTTCTTTCTTAGAGTCTGGTCTTTTAGTTCAACTTTACATGTTTTAGGCTCGT
ATTTTGAGAGAAAATAAAGAAAAAAGTATGCAGATCATGATTCTGCAAAATACTGAACTAGTGTTCTGAT
GAATTAACATGTAGCATATATAATGCTGGAAGAACTAAAGAGCAGTTGGGCTGCCATGACCAAAAGAAAC
TTCGACTGATTATAAATGTGAAAACTTGGGCCCATTCTTTGGTTTCTGTCTGTTGTTTTATGCCATGGCA
AAACTCTGCTTATTTTTCAACGTCCAACGTCAAATGGGAGAGGTTTAAATTCTATTGTTATGTCTAAACC
ACGTGGTTGTTATCTATATCTGACCGAACATTCAAGCTTTTGGTATTCCACAAGAAGGGTTTTCTTTCTT
CTTTCTTTTCATAATTGTAATGTGTTTAATTTGTTTCTCGCCCAATAATCTTCTCTGCTTCAAACTAACT
TTAATTGTTCGATCTCTTGCGTTATTTTAGACATGTCAATCACCTTTCACTGTTGAAAAAATGGTTGGTG
AGGTTAGGTGGTAGGTTTTGAAGTCTTCTAGAATAATGTGGTTTCTCTGTTGCTCTTGACTTCTTCTTGT
AGATCATTACTGGCTGGCTAAGCTATCCATACCCCCCCGCCCCTACAAATAATATTGAGTTGTTGCTGGT
CTTAATTCCTATTATCTGTTATTACTCCCACTGATTGCTTTCTGTTTCTCTTAAGGAGCT
Sequence ID: 26
Sequence Length: 3109
Sequence Type: DNA
Organism: Poplar sp.
AACTGAGACAAATATCACAAATTAACTTATTAACATATAATTGGTTTTGTGATATTTTTTCATGTTTCGA
GTTTTTGATTCATTATAATCAACAGTCCAAGTTTAATTGCTAATATGTTTAATTTTCAGATACATAATTA
TTTTTAAATTAAATTCTTTTATTCGAACTTGATTTTTAACTTTTTTATGTTTGTTATCTTCTCACTTGTT
GGTTTCTGAATAATATATTCACGGGATTCACAGCACCAACAAACCTTCCACTTTTCACTTCTCGGGAAGA
GGAATGAGAGGGAGAAATAACTAGCTCTCAAGCTATCCTTTCAATTTGGAAAGACAACCTCAGCCGGAAC
CCGAGAAGGCACTGGATAACCAAGAGAGCGAGTCACTAGATGTAAGAACCCAATATAATTATCCGTACGC
TGACACATTCAAAATGATGAAGATGCGTTTTGGGTATGTGAAGATTGGCTCGGAGAAACCTACCGCCACC
TTGGGGATAAGATTTGCTAAAAAATATCCCTATATGAATTTGAAAAGCATTATCACAGGTCAATTTGTCT
TTTGATATCTCTTCGTCCTTGAGAGGCTCAGAGTACCTCAAATCAATCCAGAAATTTTATTTTTTTTCTT
TCTAGTCCTTTCTTTTTAGTCTTCGTTATAAATTCAATTTAATCCCTTGCATCTTTGAAATTATTACACC
CCACAGCATAAATACGAAGATACCTTCACCATTAAAAGAACAATCATAGCGCACATCGATTAACGTAAAT
TGAATAGGAAACGGGAAAGGAGCATCCAAAGTTCGACCAATAGTTGAGCAAAACTGGTTTGGCTTTGTTT
TTTGTTCGAATATAAACGAATAAAGATCGACCAACAGGGAGAAAAATGATGCTACAGAAAACACGCTGAA
GCATCATCTTGTCTGGTTAGGCAAAGGAATAAAAAAGAAAAAGGTAAAAGGATCATTTGTTCGTTGCAAA
TACTGTTTAGCTGATTGCCCTGAAATCCCATGACTGAGAACCTCCACCGGGACCGGTTTCACCTCGCCAT
ACATGCTCGCTGGAAACGGGTGCGGTGCCGGCTAGTCAAGGTGGAAGCCCACCTTTGGGCTAGACGTCTT
TGGGTTCTCTTCCGCCCCTGTGTGATGCAATGTTCGGTCAACATCCTCTGTCTTCAGAAAAAGTAATTTG
CTCTTTTATCAAAAGAAAAACGACAAATTCTAGTAACCAACAGTATGCAAAGAAACGCATTGCCAATTTT
TTCTTTTTCCCTATTAAATTAACTTCCCATTTAATTTTAGATGGTTTGATATTGTAATAATAATTGTTTT
TTAAAATATTTTTTTATTAAAAAATACATTAAAATAATATATTTTTTATTTTTTAAAATTTATTTTTAAT
ATTAACATATAAAAAATATTTTTTAAATATAAAGACAAACTGGATAAAGTTCAGTTGTTTCCAATGTAAC
TTTCTGGTCAAGGAGGTAGGACCAGAAGGAGTCATTCATTAGCATGGCATTCGATGGTTTTATCGCCAGC
ATGTTTTAGTTCGGTATTATCATCAAGTAACCATTGGAAGAATAAAAAAAATTGCTTTTATTTTATTTTT
TTCGAACTCAGCCATCTTTCTCGTGTCATCTTTGAAATAGTGATTCGATATTGATATTGTATTGGGATAA
AAACCATGAAGTTATTTAGAACAAACTAGATATGTTCCTCGTACATTGTCACGGACCACACAATTTTTTT
TAATGTAAAGAAAGTGGTAAATTAAAAAAATATTAATTTAACTATCTAGGCGGTGGCTCAGTGGTACGAA
CTTGGAATCAAGAGGTTTGCTCCCTCTGTGGTCTCAGGTTTGAGCCCTGTGGTTGCTCATATAATGGTAA
CTGAAGACTTACATGGTCGTTAACTTCATGGCCTGTGGGATTAGTCGAGGTGCGCGTAAATTGGTCCGGA
CATCCACGTTAAACTAAAAAAAGATATATATTAATTCTTAATTCATTGGTTAAATTGATAATTTAAATAT
TTACAAAAGAAAACCAACAACATTAAATAAACCTATAAAAAACATAAAAAATTAATATTAAAAAAGATAT
TTATATCATGGTAAAAAGATCATTCTATCCCAAAACTAAGATAAAAATTTAAAATATCAAGAGATAAAAA
AATCTTGATATTATTATAATGAATAGTATTTTGCATCCTAGAACACGATAAATATTCATAAGAGAATTAA
TGTTTTGGATGATGAATATATACTTTTAATTTTTATAATTTAAAAATATAAAAAGTTTATTTAAAAATTG
TGTTAATGAAATTTATTTTTATATCTTTAACTTTGTTATTATAAAAAAATATTTTAATTATTTTATATTT
ATTTTTTTATTCCATAACACATTTAGATTTTATTTTGAATGTGATCAAACTCATAATTTTTTAAATGAAT
GATATACAAACTATAAAAAATCAAAGTAAAACGTACCAATCCAATTATAGCATTCCAAGAACATCTCAAA
TCATTATCAAATTACAAAAAAACACATTAACTGGATAATACACACAAGTGGCGGGATATTTGGTGGAGTA
AGGCGTGTTTGGTGGGTTTCACCGGCAGTTTTGCCAAAATTTGATTTGTTTTTGTTTTAATTTTTTGTTT
TAATGTTTTTAATGTTTTTATATCAAAAATAAATTTTAAAAAAATAATAAAAAAATATTTTAATACATTT
TACAAATAAAAAAATAATTTAAAAAACAAACAATATAACTTTCCAAACAGCTCCTAATTCAGATGCCATT
TTTTTTTTTTTTTAAAAAAGGAAAACCGCCAGCGCCGAGGACTCAGTACCCTCGTCCCTTGTTTTGAGCA
TCTAACAGTGACAGTTTCCCAATAGCGATCGTTCAAAAATCAAACACGCAATTCTCATTTTCTCTTTCTC
AGCTATTTACTATAAATTGGCCTCCTCTTCCTCATCCAAAGTAAGTAAACACACACACACACACACACAC
ACTCTCTCTCTCTCTCTCTCTTCTCCAATCTACAACCAGTTTTTTAATTCCTGATCACCCAGACCATACT
AGACCATACAGCTTCTTCTTTGATCGATC
Sequence ID: 27
Sequence Length: 3000
Sequence Type: DNA
Organism: Poplar sp.
TTACAAACAATCTTTGCTACACGTATGTACATGTTTTAATTTAATGTCACAGTTTCCTGTCCTTGAACAC
TTTCCCTGGTCAATGCTTCACTTTCTATGATAAACTATGTCTCACTAATATGTGCGCGCTCTACTTGTTG
ATTTCTGCAGATATGCAAGATGCACGCGTTCTGTTTCCATTGGTTAGTCTCCCTCTACCTCTGCCCCTCT
CGCACACTCACCCATATGCACAACTTGTAGCGGACAAATAACCTCTTTTATTGTTTTTATGATATTGTAG
TGCCACCTGCATACTATGCACATCTTGCTGCATTCCGAGCTCGTTTCTACATGGAACCGGAGACATCAGA
CAGTGGATCGCTTACAAGTGGCATGGCTTCTGGACGAGGAGGTGGAGGAGCAGGTGGTCGAGCCACTCGC
GGACCTGCTGCCAATGCTGCTGTGAGGCCCCTGCCTGCCTTGAAGGAGAATGTCAAGCGGGTTATGTTCT
ACTGTTAGGATTCGAGACAGCCCTGCTTTTGGATTGTTGTTTAAATCTCTGGGAGGCCCCTGCTTACCCT
TACATGCAATATCAAGCTGGTTGTGTCAGCGTTGGGTGTATATTTAATTTACATGTTCAAAACTGCTGCT
TTTTTCGCTTTCTTCAAACTTTGTGTGACATTTGAATGTCTTGAATTCTAAGACTTTTAAGTTGCTGTCT
TTTTTAGATTTCATGGTTATGACCAGCGCAACTCAATGGCTTCCCTCTCGTCGGGTGCATTTGTACCCTG
GTTTTGTTTCTTCATTCGTTTTGGTCATGGTAGATGTCTTTTTACTATTTGTTCGACAGCTCAATGCAAG
CTTCTAGAGTCCCTTACTGTCTTTATATCAGTAATAATCACGTTACATCCTTGAATCAAACCAAAAAAAT
CCACTATCGTCGTGAACACGGGAGATTTGGACATGGTTCGGATAAGTCTTGAGTTTTGTCTGTTGTTTTT
GGAATATAAAAGGTATGTTTGTTTTTTTCGAGAAATTATTTTTTAAATTTTTTTTATATTTATTTGTTGT
TAGAAAAGTTAATCAACAAAAAATATTTTTCAATCGAAGAAAAATTCGGCTTACCTAAAAAATTTAAACG
GAAAATACTTGCTGAAAGAAAAAAATTTAAAAATATCATATTATTTGTTAATTATATCAAATTTAAAAAT
ATTATATTATTTGTTAATTATATCAAATTTGATAATCAAACTTTTGATTGCTATATATATTTTGTTTTGA
ATATTTATTTTTTAATTTTATTTTTTAAAATTTAATTTTTATATTAACTTTGTTTTTTTATTTTTATTTG
TTTTTTTCTTATTATTTTTTTATTGAAGTTTTTTATGTATCAAATTTGATCCTCATATTTTTTTGATTGT
TACTTATTTTATTTGAAATAACTTATAAAATGTTAATTATTATTATTATTATTATTTTAATTTCTTTATC
TTTTATCTTTTTTTATTTTTTAGATTTGATCTCTATTATTTTGATTATTATTTATTTTATTTGAGATAAT
TTATGAAATTATATTTTTTTTTCAATTTCATTCTCATTCAACTTTTTAATTTGTAAGATTTGTTCCTCAT
TATTTTAATAAACTTGAAAAAATAAAACATTAATAAATTATTTTCCAGCTCATTTTCCATTACATAACCA
AACACTGGAAAATATTTTTCAATTTATTTTTCATTACACTACCAAACATCAGAAAATAATTCACTTTCCA
TAAGGAAACTATTTTCTAGCAAACAAACGGGGTCTAAGGTTCTGTTTTGTTCTTGATTTTGAAAAATAAA
AATAAAATATTTATTAATTTTAAATTAATATTTTTTATTGTTTTTAGATATTTTAATACGGTAATATTAA
AAATAATTTTTAAAAAATATTATTTTAATATATTTTTAAATAAAAATATTTTCTCTCGTTCAGTTAGGCT
GCTTCAACCAAATAAATTGCCCGGGATCATAACTCTGATTTCTGATTGTCACTTCAAACAAACGTGAGAT
GAAAAATGATGGCACTGCTAGTCGGTGATATGAACGTCAGCTTTTGTCATATATATTGGTAATTTAGAAT
TCAGTTAGTCGGGTGTTCAGCTATCTAGTTTGGTTGCTCGAAAGTAACTTCAGGTTTAGATTCAGAACGC
TAAGAAGCGTCGGCAAGGGATTATAGGTAGGTCGAGGTCGAGGTCGAGGTCGAGTGGAAAATTTTGTTAA
CCAACCAAAAATATTGAACAATCTCCAGAAAATAAATTTATTTTAACCAATAGTGTATGGATAGAGAAAG
ATCTGAAGATAATATTAAAAAATATAAGAAAAAAGAATATAAGAAACATGGTTATAAAACTCAATCTAGG
TGTCGATCTAGCCTGCATCATGAGTTGAATAGGTTGACACGCGGGTCGGCGAGTGGATTAAAGTTAGGAT
TAAAAAAAAAAAACTCGCCTCGGCCAGACTAAACTCTAGATAGATAAACTACTTGGTTGACACGCCGGGT
TAGCCAGGCGGCTGCCATGGAAGGTAGAAGGATATGATACCATGCTGAAACATGGTGATCACAGTAGAGC
AAACTATTAAACTTTGTAGAGACACAAAGTCACGTGACAGCTTGTAACCTTCCTCTAGGAGGAAGAAGAA
GAAACCCATATAAGAGTCCTCTTCATGCATTTCGAAATCAAGAAAGATTAGCATGGCTATAACTCTTCCC
TTCACTTTTTAGATATTAAGCAAAAAGGAAATCTAAATATTCTTCTTCTATCATTCACACATTCCTTTCT
TTTCTTTAAGATTTCTTTCCAACAAAGAATGGTCTTTCTCCTTCTCTCCTAGCCAAAGTCTAGCTATCTC
CATCACCTCATTTTTTGAATATATAAGCACATTATATATCACACCTATTAAAACACACAGAAGCTTCATT
CTTCACAATCTTTCTGCATTTGAAACACCCTTTTCTGTTATCATTTGTTTTCCTGCTCAA
Sequence ID: 28
Sequence Length: 3000
Sequence Type: DNA
Organism: Poplar sp.
GACTTTCGGTATACAATGAGATCTCTGTCTTGAAATGGATATCAAAGAACTGCGCCATGATTTTGAGTAA
TTTGCCGACAGTAATTGAAGAAGACAGCTTGCTGTTATCCACCATCAACAAAATCGAGAATTTTGACAAG
CCTACAGAGCTCGTGTGCACTTCCGGTGGTGAGGCACGTGCTTTCTTAGAAGCCAGTGATCTGCAGAAAG
GGAAAAATGGTTCTGAGCTGATGTTTTCTGGCAAGACAAAAAGGGTCATAGAGAGGTGGAAACTAGCAGT
TCAATGGAGGATCAGCTATAAGAAAACCCTGATTGATTGTATTTCCTATTGTACTGTAACTATCAATTCT
CTCTCAAGTCAAAACATCTTGGCCATGAGAACTAAATAATACATGTAATTTTGGATACCAGAAAGATCAT
TTAACGCACTTGTTAACGTTCTCTCCTTCATTCATCTTGTTTTAGCCACCACAAGTAGTTTTCACCTGGG
AAGCATTTGCATGAGCCACCTTCATCTTTTCTGCTATCAGGTTTCTACCATTGTTGGCTGTAGAATATGA
TTCTTGTACTTTCATTCAAGTTACTAGATGTCGACTTATTCATCATCAGGTCAATTTAGGTTTTCTAATT
GAACATTCAAATTCTAACTTTAACCATATCATTGGTTAAGTCAAGCATCTTCCATGGTTAAGTCAAGTCA
TTTTCGATTTCTATGTAAATTTATAGCTGTTTTTTAAAATATTTTTTATGAACATTAAAATTATTCAAAA
TTATATAAAAAAATAATTCTTTTCTGAATTTCTTTTTTCTTTTAATGTTCATTCAGTTGGAAGATTTCTT
ATCAAACTGCAATCCAGAGCCATGTTTATCATATCAAGTATTCACCTTCAACTAACAGGGAAATTGATAT
TAAGTCGAGATTTTTTTATTTTCTTTTAATTTTAAATAAATCAGGACGATGGATCAGAATCCTGGCTTGG
TTCTCAAAAGAGAAAAATCGCTCAGTTTATTCAGCTTCGATCATTTGGGGAAATAATCATCATCTCCATC
GCTTCAATTCCATTAATGTTTCTCCAAACTCTTATCATTTAACACTATCAAAAGTAAAATTTTTTTATTT
TTTTAATTTCAAATAAATCAGGACGATGGATCAGAATCCTGGCTTGGTTCTCAAAAGAGAAAAATCGCTC
AGTTTATTCAGCTTCGATCATTTGGGGAAATAATCATCATCTCCATCTCTTCAATTCCACTAATTTTTCT
CCAAACTATATCATTTAACATTATCAAAAGTAAAATTTTTTTATAAAAAAAATAAATAAAAAAAATTTAG
TTGAAGATGGATCAGAAATCTGGCTTGGATCTCTCGAAAGAGAAAAATCGCTCAGTTTGTTCACCTTCGA
TCATCTGGGGAAATAATCATCACCTCCATCTCCGCCTTACTAATTTTTTTTTAAAAAATCAAAATAAAAA
AAAATCATAAAACATCTCCAAATATGCATACAACAGAGGGCATAGTGTAAAAATCTAATGAACTCCTAAA
TGGGTTTTGCAGAAACGGAAAATAACATATAGCGTCAGCTCATACGCAACGCTTATGCTGTAAAAACCGC
TGGAAACCAGTAGATGAAGCATTGAAAAATAACTTAATTGACCTAAGAAAGAAAATATTCGAAGCCGAGC
AGGCAAATCAGAAGATAAGAAATTGGAAAGTCTACAGAGACGCTTATGGTTTAGAAGAATGAGGAATTGG
CTTGGGAAGAGGGGTTTATAAAGGTGAAGGAAGGGAGGGTTTGCTGCTGTTTCTAAAACCCTAGAGCAAG
TGGGCTTCTGCCACGTGTACTGACGTATAGGCAGAATAGAGTTCGATTTCGGGCTGTTTGGGCTGGACCG
TGCCCAAACCCAACAAATGAGTGGATCTTAGGAGAGTTTGGGTCGTTTCGAATTAGTCCCCAAACAACTC
AAATTATTAATTAGTTTTCCTTTTTTTTTTATGCATATAATTGTAATTCATCCTTTTCTAGAAAATATCA
AAATATTTTTTTTTATTTTATAAAAAAAAAGCAAAAAAAAAATAAAAAATATATATATATATATATATAT
ATATATATATATATATATATATATATATATATATATATATATGCTTGCAAAACCGAACCTTTTCTTTTCC
TCTCCGTTATCGTTTTGCTTTTGCTTTGTAGCAAAATCCAAAAAATACCAAACCCTATTTCTTTCCAACA
TAAGCAAAAAGCTAGTTTCGTGTGCATGCAATTGCCAAATATTAAGACTTGTGGTCCAATGTATCCATTT
TTTGTTCCCTACCACTAATTTTGGAATGAGTCAAGAGTTACCTAACTAGAAGGTCTCGAGCTTGAATATG
GATCCAAACCGGAAGTTAGCATGGTTAAGAAGCTACAACATCATTTTTCTAGCTTTCGAACAAGCAACAG
AAAAATAGAGAGACGTGTCCCTTGTTTCTTGTTGTTTTTGTGTCTTTTGTTTTGTTTGATGATAATAATC
ACGCATTTGAGAAATTTGAAGTTAATAAAGAAGTAAATTGTCATTTTTCTTGATCTGGTGAGAGATTTAA
TCTAAAAGAAAAACAAAGAGTTGAGGTGAAAGTGGCAGATTTGTGCTGGGGCCTTCACCTATTCTTTCCC
TAGTTCTTCCTTGCGAGCACGGAAAGGAAAGAATATTGAGCATTGCTTTGAGTGATAGCCTTTTTTAGTA
ACCAAGAATCTCTTCGAAGCTCTTAAAATTCCTTTCTTTTTCTTTTCCTCCTCTTGCACCCGCCTTTAAG
CATCCTTTACCCTGAGAACAAAATCTATCAGCCAATCACTTGATTACAACCAAAGCAAACCCATTTTCAT
GCACCAATCAAACACGTACCATCGCTCTATAAATTGGTTATTCTAAACAAGAGTTGATCAAAAACATTCT
GTGACCATTTTCTTACCTTCAAAACTCGTGTTGTGGTCAAAAAACAGAGCGTTACTACAA
Sequence ID: 29
Sequence Length: 3000
Sequence Type: DNA
Organism: Poplar sp.
GACTTTCGGTATACAATGAGATCTCTGTCTTGAAATGGATATCAAAGAACTGCGCCATGATTTTGAGTAA
TTTGCCGACAGTAATTGAAGAAGACAGCTTGCTGTTATCCACCATCAACAAAATCGAGAATTTTGACAAG
CCTACAGAGCTCGTGTGCACTTCCGGTGGTGAGGCACGTGCTTTCTTAGAAGCCAGTGATCTGCAGAAAG
GGAAAAATGGTTCTGAGCTGATGTTTTCTGGCAAGACAAAAAGGGTCATAGAGAGGTGGAAACTAGCAGT
TCAATGGAGGATCAGCTATAAGAAAACCCTGATTGATTGTATTTCCTATTGTACTGTAACTATCAATTCT
CTCTCAAGTCAAAACATCTTGGCCATGAGAACTAAATAATACATGTAATTTTGGATACCAGAAAGATCAT
TTAACGCACTTGTTAACGTTCTCTCCTTCATTCATCTTGTTTTAGCCACCACAAGTAGTTTTCACCTGGG
AAGCATTTGCATGAGCCACCTTCATCTTTTCTGCTATCAGGTTTCTACCATTGTTGGCTGTAGAATATGA
TTCTTGTACTTTCATTCAAGTTACTAGATGTCGACTTATTCATCATCAGGTCAATTTAGGTTTTCTAATT
GAACATTCAAATTCTAACTTTAACCATATCATTGGTTAAGTCAAGCATCTTCCATGGTTAAGTCAAGTCA
TTTTCGATTTCTATGTAAATTTATAGCTGTTTTTTAAAATATTTTTTATGAACATTAAAATTATTCAAAA
TTATATAAAAAAATAATTCTTTTCTGAATTTCTTTTTTCTTTTAATGTTCATTCAGTTGGAAGATTTCTT
ATCAAACTGCAATCCAGAGCCATGTTTATCATATCAAGTATTCACCTTCAACTAACAGGGAAATTGATAT
TAAGTCGAGATTTTTTTATTTTCTTTTAATTTTAAATAAATCAGGACGATGGATCAGAATCCTGGCTTGG
TTCTCAAAAGAGAAAAATCGCTCAGTTTATTCAGCTTCGATCATTTGGGGAAATAATCATCATCTCCATC
GCTTCAATTCCATTAATGTTTCTCCAAACTCTTATCATTTAACACTATCAAAAGTAAAATTTTTTTATTT
TTTTAATTTCAAATAAATCAGGACGATGGATCAGAATCCTGGCTTGGTTCTCAAAAGAGAAAAATCGCTC
AGTTTATTCAGCTTCGATCATTTGGGGAAATAATCATCATCTCCATCTCTTCAATTCCACTAATTTTTCT
CCAAACTATATCATTTAACATTATCAAAAGTAAAATTTTTTTATAAAAAAAATAAATAAAAAAAATTTAG
TTGAAGATGGATCAGAAATCTGGCTTGGATCTCTCGAAAGAGAAAAATCGCTCAGTTTGTTCACCTTCGA
TCATCTGGGGAAATAATCATCACCTCCATCTCCGCCTTACTAATTTTTTTTTAAAAAATCAAAATAAAAA
AAAATCATAAAACATCTCCAAATATGCATACAACAGAGGGCATAGTGTAAAAATCTAATGAACTCCTAAA
TGGGTTTTGCAGAAACGGAAAATAACATATAGCGTCAGCTCATACGCAACGCTTATGCTGTAAAAACCGC
TGGAAACCAGTAGATGAAGCATTGAAAAATAACTTAATTGACCTAAGAAAGAAAATATTCGAAGCCGAGC
AGGCAAATCAGAAGATAAGAAATTGGAAAGTCTACAGAGACGCTTATGGTTTAGAAGAATGAGGAATTGG
CTTGGGAAGAGGGGTTTATAAAGGTGAAGGAAGGGAGGGTTTGCTGCTGTTTCTAAAACCCTAGAGCAAG
TGGGCTTCTGCCACGTGTACTGACGTATAGGCAGAATAGAGTTCGATTTCGGGCTGTTTGGGCTGGACCG
TGCCCAAACCCAACAAATGAGTGGATCTTAGGAGAGTTTGGGTCGTTTCGAATTAGTCCCCAAACAACTC
AAATTATTAATTAGTTTTCCTTTTTTTTTTATGCATATAATTGTAATTCATCCTTTTCTAGAAAATATCA
AAATATTTTTTTTTATTTTATAAAAAAAAAGCAAAAAAAAAATAAAAAATATATATATATATATATATAT
ATATATATATATATATATATATATATATATATATATATATATGCTTGCAAAACCGAACCTTTTCTTTTCC
TCTCCGTTATCGTTTTGCTTTTGCTTTGTAGCAAAATCCAAAAAATACCAAACCCTATTTCTTTCCAACA
TAAGCAAAAAGCTAGTTTCGTGTGCATGCAATTGCCAAATATTAAGACTTGTGGTCCAATGTATCCATTT
TTTGTTCCCTACCACTAATTTTGGAATGAGTCAAGAGTTACCTAACTAGAAGGTCTCGAGCTTGAATATG
GATCCAAACCGGAAGTTAGCATGGTTAAGAAGCTACAACATCATTTTTCTAGCTTTCGAACAAGCAACAG
AAAAATAGAGAGACGTGTCCCTTGTTTCTTGTTGTTTTTGTGTCTTTTGTTTTGTTTGATGATAATAATC
ACGCATTTGAGAAATTTGAAGTTAATAAAGAAGTAAATTGTCATTTTTCTTGATCTGGTGAGAGATTTAA
TCTAAAAGAAAAACAAAGAGTTGAGGTGAAAGTGGCAGATTTGTGCTGGGGCCTTCACCTATTCTTTCCC
TAGTTCTTCCTTGCGAGCACGGAAAGGAAAGAATATTGAGCATTGCTTTGAGTGATAGCCTTTTTTAGTA
ACCAAGAATCTCTTCGAAGCTCTTAAAATTCCTTTCTTTTTCTTTTCCTCCTCTTGCACCCGCCTTTAAG
CATCCTTTACCCTGAGAACAAAATCTATCAGCCAATCACTTGATTACAACCAAAGCAAACCCATTTTCAT
GCACCAATCAAACACGTACCATCGCTCTATAAATTGGTTATTCTAAACAAGAGTTGATCAAAAACATTCT
GTGACCATTTTCTTACCTTCAAAACTCGTGTTGTGGTCAAAAAACAGAGCGTTACTACAA
Sequence ID: 30
Sequence Length: 3000
Sequence Type: DNA
Organism: Poplar sp.
TAGAAACTCACTTATCAAATGAATTGAGTTATTTTTTTTTAAAAACAAAATCTTTGTGGTCGCATTAGAT
TTTATAACCATGGAAACGAATGAATCTTTGGGGGAGGAGAATTTGTTTATTAGAATTTTGTTTAATTTTA
AAGTAAATTTTTAATTTTTAAATCCAGATAGTTAAATGCATAAATAAGTATTACATAGATAAATATTGGA
TATGAGTATTTTATTATTATTAACATAAAATCTAGAGATTATCCTGTAAAAACAATTTAATTTAAATTAA
TTTATTACCTAGCAAAAATATTCATTGTGATTAATAAAAAAAATTGATTATCTCCAGTAATGTATCTTAA
TCCTTGTATTTAACACAATAATTTAGATATTTAAATTAAATTTGAGTATAATTTAACGATGAGAAAAAAA
AAAAAAAACTTTATACTTCAAGTAATGTATTTCCGTCCATCATGCGCGGGAGCTGGAGAAGTTGAATTAA
TGATCTTGGCGACGAATTAAGCATGGCAAGTATTGGCAATGTTGCCGATGCAAAGAGTGCCAAAAGCAAT
ATTGAGAAGATCCTGATGGGCTTCAACCCCATTCAAACCCGAAGAAGTTGAGGATAGTTGGATATGGAGG
TTCAAAATTTCCAATTGGATGATGAACATGCACTTGATGCAACGAAATCTAATGGAGATCTTGCTGGAAT
GGAATGGTGTGGCAATTCTGAACAATTTCCACCATTTGGGAAACTAGAGTTCCTTAAGCATCTTTCTTGG
AAAGTCTGCATCTTGATTCCCTGAAGAGTTTAGAGCAATGGACACCAGTTACTGAAGACGGTTTTCCTAG
CCTTGGCGAAGTCTAATTGCTCTACATAGTCGAAATGCCACCAGTTATGATCTCCATCAGCACATTGAAA
ATCGTGGAAGGCAGTGCTACATTATTGAGGTCAGCTAGGAACTACACTTCTCTTATCTTCCTTGAAATTA
AAATGATTTCGGATTCGATGCTTCTTAATGATGACTGGTTGCCAAACCTTGCACTCCTTGAGCACTCACA
TGGTTTTGAAAAATCTGCAGTCCCAGTCATATCAGATGTTAGACAACCTATCTTCTCTTCGGAACTTGCA
CCTTTCAGTATTCTAATAAATCTGCATCTCTACCTGAGGAGTGCAATACCTGACAGCACTTGAGAAATTG
TATCTGGAAGCATGTCCAGAGCTGATTTCGTTGCCGAAGAGCATCCATTATCTCACTTCTCTCCATTATC
TTATTATTCTGGATTGCAGGGCCTTACGTTCTCTACCAAATGAGATTGGACATCTCACCTCCCTTTCACC
GTTGTTGATTTGGGACTGTCCTAAATCGATGTCTCTGCCAGAACAAGTACAGAATCCCAGTATGCTCAGA
GTTGAGAATAAAGGGGTGTCCATATCTGAAGAAGAGGAATAAGGAAGAGAGAGGAGAGGACTGGCCAAAG
ATAGCTCACATCCTTTCACTGATATCAACCTGCAGAAAGTAATCCAATGATCTCTGCAGACATGAAGATT
AGAGTGGTTGACTTTTTGGATTTCGGAATATGACGAGGTTCACTCCACATTAGTTACATTGGAAGTTCAT
GATGTAAGCCTTTATGCGATTCTACATGGGAAAAATGCTGCAATATGGTTGCCTTGTTCTACTTTTTATG
ATCTATACAGATGAGATGATACACTAGTTATAATTTGCAGAATCAACGATGATAAGTTGTAGTCCAGATT
TTTATAATCTATAGGGATTATCCATTTGATGTTTGCAATCTACCTCTGATTCTCTTTACTGCTGCATTGG
GTCAGAATTGGTGGATGTATCTTCCAAAAATATATTTCCATGTTTTCCAAAAATAATAATCTATATGGAT
TGGTTATTCCAGCACCCATAAGTCAGCAACCAGAGGGAGTTTTCCAGGAGGAATTGGACTGGAAGAAAGA
GGAACAATACTATAAGGAAGAAGAGAAGATAGGTCCAGAAACAAGAGGCATTCTATGCGGCAGAAACACC
ATATCTTTTTCAATTCATTTTTATTTTTATTTTTTACCCTTCCATGTTTCTCACAGCCCAGTCATTTTGA
TCTAGCTCTTTTAATGGTGACAATGCTTCATCCATAATCCATAATCAAAATTCATATGTTTATCTCTCCT
TTTTTTTTTTTTAAGAATTAATATACTTTTCTTATCAAATAACAAAAGCAGAAGAAAAAGGGAAAAACCA
ATATGCTTGCTAAAACCAAACCTTTTCATTTTATTTTATTTTTCTCTCAGTTACCATTGCTTTTGCTTAA
TTTATAGCAAAATCCCCACCAAAAAAAAAAAACAGACCAAGCCCCATTGTTTCCCAACATTATGTCCCAA
TAAATTATCACATGTTAATTGCAATTGGCAAATGATAAGACTTGTGTCTGATGCATCCATTATTCCTTGT
TCTAATTCTTTATATATTTTTTACATGTTTTTTTTTTTCATCCAAGATATTAACCAAGTATAAACTAAAA
ACTTTAATTGACCTGTAATCTTTTAAATATTAAAATTAAAAACAAAAACAAAGAAAGACCTTGGCTACCT
TTCCTTCCTTCCTTCTCTCGTCTTTCTTTTTCTATGAATTCTTCCCTGTTCCTATCTTAGGAACTCGAAA
AGAAAAGAATATTGAGGATTGCTTTGATTAGTCTCTTTTATAACCAAGAATCTCTATGTAACTCTTAAAA
TTCCCTTCTTTCCCTTCTCTTTTTCTTTACTAAGAATGTTCCTTTTCCTCTTCTATCACCTATCGTCCAT
CGACCTATTCCCTTTTAAAAAAAAAAGAGTAAGTCTCAAAGCCAATCATTTGATTACAACCGAAGCAAAC
CCATTTTCATGCACCTACCATTGCTCTATAAATTGGTTGTCTTAAAGAGGAGTTGATCAAAAAGCCTTTC
GTGATCATTTTCTTTCCTTCAAAACTTCGTGTTGCTACAAAAAATAAGTGTTGTTACAAA
Sequence ID: 31
Sequence Length: 3000
Sequence Type: DNA
Organism: Poplar sp.
GTTTGTGCTCAAGCCCACCGACAAGAGCTTATCCAAGATTTATATAAAACATGGCAGGATCCTGTACGAG
GGACAGTGTCTGGTGGCATGATCAAGTATGATTTACCTCTCCTCTTGTACTTCCTCTCTTTCTCTTCATG
CTTGTCTTTGGTGCATTAAAGTTGGGATTATTGTTAGTTGACCATTGAGTCAACAACTAATCTGTCTCTG
TTCCAAGAATAGTTTGTGGTTAGATGGTTTGAATGTATTTTTCTATAATGCAGGGAGCTTCTCATATCCT
TCCGTAGGGCAACCGGGCAGAAGCCACAGCGAATTATATTCTACAGGTATTATCCTGTTCTCCCTGTTTT
TTTTTTGTGTATTGCTGCCATGTCTAATGGTGTAATTATTTTCCAGAGATGGTGTCAGTGAAGGACAGTT
TTATCAAGTTTTGTTGTACGAGCTTGATGCTATTCGTAAGGTAAAGCTGCTTTTCCTCGAAGCTCTCTAG
GTTTTTTCTTTTCTATTTGGTCATTATCTTGTTGGATTTTGTAAGCCCTCTTAATTTCCCATTTCTCTGC
TTGTCAACCTCAGGCATGTGCTTCTTTAGAGCCCAATTACCAGCCTCCTGTGACATTTGTTGTGGTTCAG
AAGCGTCATCACACAAGGCTGTTTGCAAATGATCACCGTGACCGCAATGCTGTTGACAGGAGTGGGAATA
TATTGCCTGGTAATAAATCTGGCTTGGTGATAGAATTGTGTTCCAATTTGCTTTAGATTTGCGTGACATG
TTTCGCTAAACACTTCATATGGTAGGTACTGTTGTGGACTCGAAGATCTGCCATCCTACTGAATTCGACT
TCTATTTGTGTAGTCACGCTGGGATTCAGGTAAGACTCGCATTTCTGCGCAAGATTCACATTTCCTTGTG
TAAATCTGATTGCTGTAGAGCACTTGATGCTTTTGATTTCTCACCAATGGCCACCAACAACAGGGCACAA
GCCGTCCAGCTCATTACCATGTACTTTGGGATGAGAACAAGTTTACTGCTGATGGGCTGCAGTCCCTGAC
AAACAATCTTTGCTACACGTAAGTTTATGTCTTCTTTAATTTTACAGTATGATGTGTCTTGATAGTTTAT
TGATTTGTTTCCTTTCCTTACTTGTCATTCTATTTTTATTTTTATTTTTATTTTAGCTCTAATTTTGGTC
TTGCAACTCTTTTTTTTCCCTTCCAGATATGCACGATGCACGAGATCTGTTTCCATTGGTAAGGACCCCC
CCCCCTCCCCACACACACACACACAGCGCCCCCATGATTCACGTCAATACAATCATGGATTGTACTAGAC
TAATAACTTCCACTCTTGCTTGCTTTATGATGTTGCAGTGCCACCTGCATACTATGCTCATCTTGCTGCA
TTTCGAGCTCGTTTCTACATGGAGCCAGAGACATCAGACAGTGAATCAATTGCAAGTGGCATGGCTGGTG
GTCGTGGAGGTGCTGGTGGGGGTCCTCGTCCAACACGTGGACCTGGTGCTAATGCTGCTGTGAGGCCTTT
ACCTGCCTTGAAGGAGAATGTCAAGCGGGTTATGTTCTACTGCTAGGATCGTTGGAAATCCCCTACTTTT
GGATGGGCATTTAAATTTTACTAGTTGAAACTGTTCTGTTCGGAAAGCGGGGGAGGCCGCTCTGAAGCAG
AATATCAAGCTGGCTATGCTCTGCTTTTTGTGTCATCTGGCCAGTTGATATTTGCTTTTAAATTGCCACT
GATCCTACCGTGATCAGAAAGCTGCTTTTTGCTCATGCTGTGTGTATGGCATCGAATATCATTGAATCTT
AAAACTCTAAATCCTCTCGAAACTGGGATTTCATTTTTCATACATCTGATCATCTTCCTGCCACCTCTAT
TTGTCTCGGCATGGATCTGGAAATTGCAGTTTGGATTTAGTTCCAACAAGCCAAGGTTCCAAATTTAGTC
AAATGGTAATCATTAACAGACGAAAGATTAAAGATTTTGAGAAAGCTAGTTGGTGATAGGGAATGTGGGC
TGTCAGATACTTCTGGCCTTCCCAGTCACAACAGTTCATGGCGTCTGCTAAGCCAGTATTGTTGCCGAGA
AATAGCACGAGGACTCTTCATTATGCAGCTCGGGACTGGAAAATTGTATAGAAGCAGCAGAGAAGTCTCA
ATCCTTCAAGTAATTCGAAGTCGTTTATTTAAAAATTATTGTTGGTAAATGAATGATAATTGTAACGAGT
GATATAAAAACAATTTTAAAATTAAACTCAAATAGAAATTTTATCCTTCGATCGAAACAAGATTTAATCC
CGTTTTATAATCAGCTTTCGATTTAGCCCAAATCAAGATCTAGAAAATAATTCAAATCAATTTAGAAAAA
TCACATGAAGCATGATCCAAATCTACACTATCCAATGATGCTCTCTCATAGGCAAACATCCATGCGAAGG
AATAAAATAGTATCCAAAGAAAGAAGTAGCTTGCGGTCGTAGCAAAGAACATGAGAAATGAAAAAAATAA
TTTTTTAAAAATAAAAATAATATTATATTAATAAAATTTTAAATAAAAAATAGTTTGAAAAATAACTATT
ATTATTAATTTAAACAGTAACAAGATACTGAAACATGGAGATCACACACAGGTCACGTGAGAACTTGTAA
CCTTCCTTTGGGAAGGAGAAGAAAAGCATAAAATATACGAGTCCTCTCCAATCCATGCATTTTACTCACT
GTATCATTCTTCCCCTCCACTTTTAAGATATTAAGCAGAGAAGGCAATCTAAGCTTGCTTCCTCTGTCAA
TCACACATTCCTTTCTTTTCTTTAAGATTTCTTCTCCTCCTCCTCCCCTAGCCAAAGCCTTTTTATCTTT
CTTTTTTTTCTAAGCATTTCCAGCTCCCTATATAAGTACATTGTCGCACACCTTTTACAAACACACACAG
GTTGCTTTGTGTTTTTTGAAAAAAAACCCCTTTTCTGTTATCATTTGTTGTCCTGCTGAA
Sequence ID: 32
Sequence Length: 3083
Sequence Type: DNA
Organism: Poplar sp.
AAAAAAGGGATAAGCTCAAAACCCTATTACAAGCCCTTCCAGAAACAATTGCAAAGAGTTTGTAGCTGTA
GTCTTTAGCAAAGCAAAATCAAACAACAGTTTATCACCTCTTCCTTTTCTTATTTCCGGCAGCACTGCCT
TTCTTGGAGCCTCTAAAATCATCAGTGGTGTCAGCAGTGTTCTGCTTCTTTAAACCTTTCCTCCCTCCAA
ATCCAAATTTGGAATCCCTTGATTCCCTCTTGAATTTTTTCTTTTCTGGTCCGTTTTTCCCCTTCTTCCA
AGGTTGTCTCCCCTTCCCTCCAGACCGATCTCCTGGAGACACCCCTGGCCTCTTCTTGTTCGACCTTTCA
AACACTTTTCCATCTTCAAAGGGCATATCCAACTCACCATCCTTGTCGCCTCCAGCAAACCCACTTTGTT
TCCTCTGTTTCCTCCAGTTCTTAACAGTCTCTATCGCTGCCTTTTTCTGTGCATTTCTCTCTTTCTGCTT
CTGGGCCTGAACCTCTTTAGCTAGTTTCTTAGAGTCTCTAGCTTTCCTTCTCTCCTCAGCCTCCTCAATG
CTTCTCTTCTCTGCCAAAAGCCGGCCCTTCACTTTCTCCATATGAGTATCCGACTTTGCCATCTCAGCAT
AATAATCAGGAGGCCTGAGAAAAGGAATCCCCATCGACTCTAGCTTAGCATATGCCTGCCTTGTTCCCTC
TAAGGCTTGTGTGTAAAAAGCAAGCTCTCTGGTCAGGTCATCATTCACGTCCACCTCTTGCTCTTGATTG
AAGTCGATCACAAGTTTGTGGATCCATCCAACATTCTCTGGCCAACTAATGTCTTGAAGCTTGTCAGCAA
GACCATCTCTATTGAATATAGCATTTTTAGATGGTTCAGGCAACTTCACACCTTCATCCTCCGATTCTGA
CTCAGATCCAGACAATTTTTCATCAAGATTATCCATTTCATCATCTGTTATCAGCTCCTCATCAGACAGG
GTCAATTCTTCCTTGTTTACCACCATTATACAAACCTATCACAATCCAAAACCGAATTACAATTAATAAG
CACACCAATATCCACTCAAGCATGAAAATAGCAGATATTACTCATGGATATCAATACATAAACTCAAAAT
TAAGCTAAAAAAGGTACCAAAGAATAACATAGTGACAAGTTCTCCTCAATCAAATTGGATTCAGATACTT
CTACAAAGTAAATTGGCGCTGGTATACAACCCCGGATAAAGCAAACCACCAAAAAACACAAAACTCAGAT
TGGCTCAACAAATCAAGATTAGATAATCCAATTTAAATACTTAACCACCAAGAAAAACCCTTAATTTATG
CAATAAAATTCGCACTAAATTGATAACAAGACCATCAAACAAATACAGACAGAGCCTAAAATCAACAAAC
TCTAATGACCCAATTCAAAATTCTTCAAACCCAGATGCTATTTTAGCATAAAGTTTATTACTTCCAACAA
AAACAAGCTAAAACACTACTTAAAGCGAAAAATCGAAGATGCAGAAAACGGAGCCATTAACTATACAAAA
GCAAGCTGTTTCGATATAAGAAAAAAAAAGGTAGCGTGAAAGAAACTAAGCAGGTTAGGTTTAGAGAAAA
GGAGAAAAGAAACAATTTACCTTGTTAGAGAGTGAGGGAAGTTGAGATTCGTTGAGAAGAAGATTATATT
GTAGAAACTGCAACCTAATAGGGTAGCAAGAAGAAGCATGAGTAAAACCCTAAACCCTAATTTGACAGGG
CACACCTGGGCCTTAGCCCAAACCATGTACGATTTAATGGGCCCACGATGAGCCTTACATGTGGTCTTGA
AATTTTTTGTCACGAGGTGTTGTGTTGTTTTAGAAAGTAGTTGATATTAGGGTGATTAAATTAATTAAAC
TATCTATAATATTTATAATAACCGACTCGAAATTAATTAAAACTAAAAGGTTTATTAAATTTTCTATATA
ACTTTAATTATAAACTAAAAACTCATTAAATCAAAGTTCAATAAAAAAAAAATAACCCAACAAGATACTT
TTTAATTTAAATTAAATTTATTTGATTTTAACAAATTTAAAACCAAACTGAGATTAAAGTTTTTATTTAG
TTCAGTTTATTTTAATTTTTTTAAAAAAAATTAAATCTTTTGGTCCATCCCTACTTGATATTTTCATGCC
TTTTTCTAATTATAATCTTTCAATCCAAAATATTTTTTATGATAAATTCTATGGTATAAAGCTTTTATTT
AGCAATGAAATGCCGAGTTTAAGCACTTTTAACCCCAAAATTATTGATTTAAAGCTTTTTTTTTTCATGG
GATGGCTATTGGGGGATGTTAATTGTCGGCAGTAGGTTACATGTTTTTAATTATATTAATTGTATTTAGT
TGTGTTTTAAAAATATATTTATTTTAAAAAATATTAAATTGATATTTTTTTTAATATTTTTTTATAATTT
TAATATATTAATAAAAAAAAATTTTAAAAATATTTATTTCCAAGTCAAATCACCTTTAAAAATTATTAAG
CAACGTAATTCCAAACACGCTTACTGAGATATATTATCCCACCAAATTTATTTAAAAACTACGTCGTTTT
ACTTAAAAAAAATTTAAGACTATAAACACCCTTTCTTTTGCAGGACTTTTGTCGGCTGGAATGCCTTCCC
TTCCAAGTGGGCAGATAAAAAACAAGGGGCGGGCGCCGCGCCCATGATCGCATGGATATCATCATGCATT
ACTGGACCGCCCACAGTAATATCTTCAACTTTTTACCCTACGAATTTTCAAACAAAAAATTCCCCGCATA
TTCCCCATTGGATGCAAGACCCCTGCCGCCGATTGGGCCCACTCAACCATAAAGAGCCACGTCTCACGTG
ATGATCGAGAAACCAATCAAACCATCCACATCAGATCTTGCCTCAAATGTTCAACGGCTTTGATCTTACC
TCCCGTGGTTTTTTCCCCACGTTAGGACTCAATAAATAAAACCTACTTACGCCCTAGTTTCAGCATCTCT
GTGACTGTCTCAGGAAAAAGAACAGAAAAGAGAAACCTCAGGATCATCGAAGTGTTGGAACATCAGATCA
ACA
Sequence ID: 33
Sequence Length: 3109
Sequence Type: DNA
Organism: Poplar sp.
AATACAGACACCAAGGATACTTCTAAACTACAAAGGAAAAGGAAGGCAAAAGAGCTGATTTATGATGCTG
AGAACAACCCCTTGAACCCTTATTTATATTGAATTTTGACACTACAATTGAAGTCGAATTAATTAATTAT
CAAAATTAATTCTCACCATCTTTTAACCCTTAATTACCCACCACGAAACAAAATAAAAATATAACTATTT
AATCATCTAAATGGTGGTCCAATGATAAGAGCTTGGAACTAAGAGGTTTGCTTTCTCTGTGGTCTCAGGT
TCGAACCCTATGGTTGTTCATATGATGGCCACTGGAGACTTACATGGTCGTTAACTTCAGGGCCCGTGGG
ATTAGTCGAGGTGCATGCAAGCTGACCCGGACACCTATGTTAAACTAAAAAAAAATATGACTGTTTTACC
TTATAAATAAGATGTTTTCTTTTAAAAACAAATTGCTCACTTAGTGAAATGTTAATAATCAGGCATGTTA
GTTATGGATTGGTTAAAAGATAATATTTCTTCAATTTATTATCTAGGAAAAGAGTGGTAAAGGTAAGAAA
AATTGGAATCTAGGAAGTCAAAAGGTCGAAATGTCCCTTGCAATTTCTTCAATTTCCCTTTTATTTAAAT
TATTTTTCGCAGAAAGCAAGAGCCATCGTATTTCAGGCAGCAGAGGGAGCTCATCTCACCAAACTCTTCT
TGACGGTTACCAAACCCCGCCAACAGACAAACAAGAGAAGGTCAGGTATACTTACCCCTTCATGTTTAAA
ATGATGCTTTGATGCATAGAGTTTTTTTCGATTAGAGAATAATATAAATTATATTTTAAAATATCATTTA
ATAATTTAAACTATTAAATTAAAATAATTCTTTAACATGATATTAAAACCTTAATAACCAAACAGTCACG
AATTCAAATTTCAGTGCAAGTTTTAAACCCAAAGAGCTTTCACTTAAACGAGTATGTTAGAGAATAATAT
ATATCATATCCTGAAATCTCACCTAACACCTTAAATTATTGGGTTGAGATGATTTTTTGACAACCACGTG
TATAAAAAAATATGTTATAATTATATATCATATGTCAAATTTAAAAAAATGAAAATATAACACCATTAAT
TAAAAGAACTAAATAAAATAATTCTAATAAAAACTTACATTAAAGGTCAAAATTAGTTTCAATTAGGCAG
GAAGTACAACAAGACTAAAAATACTTAATAAAAACACCTACTCTAAATATAGCTTAATTATCGGCATGAA
AAAAGAAAAAAAAACTCTGATGTTTAACCAAAATTCAAGCTTGAAATGGGAAAAATTCAAATTGGATTGA
GACGTAATAAATTATAAATACAAGTATAATACTATTACCAACTCATTAATTTTTCATAAAATTACTTTGA
GACTTAAAAAATGAATAAAAACTTATCATGTAATTGTTTGTAAAATTGTCATTAATGTATAGAATAAAAG
AGAGACAAGTAGAGAAAGTTATTCAGCATTTAAAGTAAATAAATCATAAATCTAAACCTAGTGCTATATT
TTTTTTTCTTATTTTTTATATTTGAATTTGTCTGTATTTTATTAATATTATATTCATTATAAATAAAATT
AATTTTATTTTTTGTTAGGATTATTATCAAACCGAATTAAGGTAAATCCATATTTGATTTATTTAATAAA
TAGATTAGCGGATCTAGATCGTATAAGTTAAAAATCAAATCCATTTAATTATATCGTATAATTCGAATCA
AGATCTAGTCCATTGATAACCATAGTTGCCAATGGCAATAAAAGTTTTACCCATAAAAAAAGTGGGATAA
CTTTATTTATAATAAATATAATATTAATAATGAATATAATATTAATAAAATTATAATGACCTCTGTTTGA
AGGTAATATCCTAACAAACGTGTGACAACAAATTATTAATAATATGAATATAATATTATATTCATTATAC
ATATAATATTAATAATGAATATAAAATTATTTTTCGAGGGATATACTTTTCATTATTATTAATATGAATA
TAATATTCAATTGTTTGTTAGGATTAATATTTAAAAGTTTTCGAGGGATATACTTACTAAAATCCATGAC
CTCTGTTTGAAGCCTTGAAGGAGATCAACAATAATTGTTTTTAAATAGTTTTTAATTGATTTCAGTAAAC
TTTTGGTGATTCTATTTCTTTTCTTGTTAAATAGTTTTTTCCATCAGTAAAATATTTATTTTTTATAAAA
TATAATAGAGTAATTAAGATTTTAAGTTTTTATATAATTTTTATTGGAATTAATTAGAAAACAATATTTC
TATAAAATGGGATTGTGTCAAAATAAGAATTATAGGAAAGATACTTATTAAATACAAATTATACTAACAA
ATTAAGGGAAAATCTAGTCGATTAATAAAACTTGTATTAAATGGGAAGCAATATTAATAATTTATGGAGT
GAAAATAACATCACTCCAGTATAATAACACCATATAAGTGATAAAAAACAAAAACTCATCAACCCATAAA
ACATTTATTTAGGGCGATTCTATGATTTATTCATAGAAGCAATCATTATTAACCAAATCATTTTTTTTCT
TGACATCTCGATAACCTAACACCAAAAAAGCCAAAATAAACCATTAAATTCAATTTCACATTATTTCAAT
GTAAAAGAATTAAATAAAAAAAAACTATGGAGCACTGTTATGTTCCATAATGATTTTATGTCTTATACTT
TAGTAAAATCATGAGATATTTTAAATTTCTCTTAATAATAATAATTTTAAGTTTGAGATTCTCATTTGAA
AAAAGAATTATTTTTAAAGTGTAATTTGCTTTGCTTTGCTTTGAAAGAAGTATGGAAACCAAATAATTAA
AAAAGAAAAAGAAAAGGAAACGACGTGATTTCTTGTAGTAGAAAAGAAATAAACCAAATCGTGGTGTGTG
TTTGGTACTGAGGTTGAGTCTCGTGTGTGTACAAATACCTGTGTCTCTCTAGTGTTCACTATCTCCTCTC
AAGTCCTAAGTAGCTAAACACTTGTCTCTTGCGCCTCCCCCTCAAATCTCCCTCTCCCTCTCTCTTTGTC
CAGATCCAAAACCTTATTTAATCAAAGCT
Sequence ID: 34
Sequence Length: 3000
Sequence Type: DNA
Organism: Poplar sp.
ATTGGTTAGAGACTTTATTTGTTCTTTAATTCATGTTGCTTATCATATTAACCCTTTTTTTATTATTCTT
TGTTCTAGCTTGGTCAACAGGACTTAGAGAATGAATTTGATATGGATATTGGGCCAGAAATGAGTGTTGT
TTATGTTGCTAGCCCAAATCACTATAATATTTTAGTTGTTAGGATAAAAAACTCTCATCCTTCATCAAGT
TTGCCACATTAGTAACTTATTCATTGATCTATAACATTACCTCCACCAATCCTTAACAATATAAAGAGTA
ATTTGTGTACTTCTAGAGCTACTACTACTTTTGAGCCTTCAAGAGTAACTAGGTAATATGTTGATGACAT
AATAGGCAAGATAAGATCTTTCACTAAACCATTACATTCCTAACTTAGCTGGTGAAGTTGAGAAGATCTT
CCACTTTAACGAAAATATAAAGATTGATTGTTTCTCTATCAGAAGAATGGTGACAGAGGTGATTGAAGAT
GCTAAAAAGCTTAACCATTTAGACTCTTCACTATGTCCTGTGATAACATCATAGTAGCGAGCCTTAGAAA
TTGAAAAGCTTAAAGGGCAGTTCGAAAATTTCAAAGCTCTTAAAGATGATATCTCTATATCGTTTTCCAC
CACTCATGTTGGGATAAAAAAACACAGAGGAACAACTCATGAAGCTACAACACAAGGACCAAGTTAAAGT
CATCCCTCAAAGTGCATGAGGAAGAGCTTTTTAAGAGAAAAGTCACCTCTGGCATTAAAGTATATATCAC
CACAATTTCTACTTTCCTTGTACAAAGTGAAGCTGATGCAGTCATTTTTTAAAAAAATTAAGAGGACTAT
TGGAGACCAAACTAAGAGAGTTATATGTACATAACTAGAAACCATGATATGCTAATAGTTTCTCATATCT
TAGAAATTTTTTTTTCCTATCATTTCTTTTCCTTTGGAATGCTTTTTAGTTTTGGTAAAACAACCCCTTA
GGTCAAAAATCCCTCAACAAAAAAGTAACTAACCAAACAAAGTCTACGTACCTGACTCAAAACAAAATTA
ATCGGGTTGACTTGAAAACACTAGTTCAACCTTGAACTAAATCGAAAATCATGGTTTAACCCGAAACCTT
TGATTTGACCATAAACCAAACCAAAGACCACTGGTTTGACTCAAAAAATAAACCAAAACTTAAACCAGAA
ACCAAAACGAATCAAACTTGAAAAAAATACAAAAACTCAAAAATTATTTGTCAAAACGAATCAAAACCCA
TAAAAAATAAAGAATACGAAGAACACCAATGAACATTTAAACAAAACCCAAAAACACAAATTTCAATGGA
GGTGGTGTTGCCTTTTAATCATGTCAGAAAAAGTAGATGAGGGATTGTAAAGTTTTTTTTTATTTGATTT
GATTTTTATTTTTTAGAGTGATCAGATGATATTTTGAGGTTTAAAATTAGTTTATTAAGATCCTAAGGAT
GCTTTTGGGGGTGTTTTTAAGTAAAATCAAGTTGAAAGCTAGGATTTTGTTGTCCAAGTTAAAATTGTAA
TAGCGGACAACACGTTATTTGCATAAATAGACGACGTGTCGTGCACACTATTTAAAGAAAAAATATTTGG
TATTGTCTGTCATTTTGTGTGTTGTTTTTTTTTAAAAAAAAAAAGAGAGAGAGAACCAAACTCATGGGCA
TGCCCTTTCAAACATGGGCACGCTAGGGCTTTTTTCCTTTTAAGCCAAGCGTACTAGCCTAGCCTTCTAA
GCTTAGCAAATCCAGGCATTTATCTTTAATTTTAATTTGTTATTTTAACTTAATTTTATATTAATATCTT
CTTTCTCATGTTTTTTTAATTGTTTAATTTGAATACTATCTACATATTAATTAGTCTTTTAAAAGGTTTT
TATAATTTTATAACACCTCAAAGAGATTATAGTAATTTATATTTAAATTTTTTTTTCCTTTTTAAATTTT
AGATAAATTAGTTTTATATGCATTCTGCATTTATATGAAATATAATCATTTCTACTTAGCAACCAAACTA
AACCTTTAAGATAAATACATGAAAAATATATAAAATAAAAAGTGTAAAATATTTGTATTTTTATAATTCT
TAGCTTTTTGCTTTGAAAGTCAACTTTTCATTTTAGTTTTTTAATTAACTTTCACACTTTTTTTTTCTTC
TGATTTATCAATTAATATATTTTTAATTAATTATAATAAACAAATACATTATTTTTTTCATTCCTCAGTT
AACAAATATTTTTATACCAGAAACCACATGTACGAAGCCGGTATTTTCTTCCCTGTATTAAAGATTATCC
TCAATCCTACTCTCATACATTAACTAGTAATCACAAATGATTTTTAGGAGGAAAATAAAAACTCAGCCCG
GACCGGCCCATCACGGCCTGGACCGGCCCATCACGGCCCGGACCGGCCCATTTAAGTAATTACCTGCCCA
TATAAAAACGAGCCTCCCAAAACAAAACCTAGTACATTGGATGGACGGGCCCATATCCAAAATGACAAAT
AATTCACGCATGATGGGTGGGACCCTGTATTTCTTAATAAGAAAAACAAAAGAAAAAACACAGGCACCCA
CCTAACCTCCTTTTTCTTTTTTTCTTTTTTTACTGTGGTTGACTTTTTGTTAGTTTTATGCTTTTGTTTG
GGATTAATTTTTTTTAAAAATATATTTTTTTATTTAAAAATATATTTTTTTATAATTATTTTTATTTTTA
ATGTTAACATATTAAAATTATTTAAAAATATTAAAATAATAATTTGATTGTTGACACATTTTATCTTGCT
CTTGCCCCTTGAGCTTCCAAGTGAATATCCAACAACCATACTAATACATTGTATTTTATTAAAAACAAAT
TGAATTATATTTTAAAAAAAACTAAATTATTTTTTTTAAATGAATATATAAAAAAATAAAAAATATTATT
TTAATATATATTTAAAGAAAAAATATATTTTAAAAAATAATTTATACAGCAATACAAAAT
Sequence ID: 35
Sequence Length: 3000
Sequence Type: DNA
Organism: Poplar sp.
TCCAATCCAGATTCTCCGTGTAGGGCAGCTAGTCATCCTCAGTGCACCTGGAGGTACCTTTCATTGCTTC
TTTCCTTTGTTTTCCTATAAGGTAAATGCTTTCATTAGCAGTATCATGTTTTATGATCAAACTAAGCTCT
GAAGTTGAATTGATGAGAAACTGCTGATATTTTATAGCCATAGATTATAGAACATGCTAAGATCATACCT
TTTGTCTTTTACCAAATGATGGTGATGAGCACGCTGCTAAGGTTGTTACTCAGGCTGGGATGGAGGATGT
GCTGTCAACCTGAGTACGGTCCAACTTTTTGTCATTAAATAGTCTGGTCCGGCTCAAATGAACTTATTTG
TGTAAATGGACATGTTGCATGGTGGGTTGAACTTCCATTATGCAAAAAATACCCCTGGAATACATAACTA
AATTTTGCTTCAGGCTAGTCCAGCTTCATGTTGTGTTGGGACCAGCGCCAAGCAGGCAAACTTCATACTG
CAGCCTGTGCCAAATCTTTTGTTCCCCACTTTCATTGCATTTCAATCACTGTACCTTTTGTTCCTAAGAA
AACTATTGTGGCATTAAACATGCTAGGAGTCATTCTTTCTTTGTTTTGATTTGGAGTGCTATTTAACTGG
TTGATGATAACTTTTTGTTGAATTTACTTTCCAAGCACCTTTGTGCATTGTTTCCTTGATTAAATGCTTC
AACCTATGATTTTATGAGCTCAAGAATTTTCTTCTTGATTATTTCAAACAGAATTTACAACCATGGCTGG
GAGGCGTCTTAGAGATGCTGTCAAGACAGTGCTCATGAGTGGTGGGAATAAAAAATTTAACAGTAATGTT
CACGTTGTTATAGCAGGATTGACTAACACCTATTCACAGTACGTGACTACCATTGAAGAGTACGAAATGC
AGAGATATGAGGTAAGTGAATAGTTTAATGACAGATGCATGAATAATTTAATCACACAGTACATGAATAG
TTTTCTTTCTTAGGAAAAAGTTCACTGTGATATGTAGTTTTGGCGTTAAGAGAACTATATACTAGCCAGC
TTGACACGCACGGGCAGTTGGTTCATTAGCAAAAATAGGTTGCCATCTGGTTCTAAATTGGAAGCTGTCC
GAGGCTTTCAAGCACCATCCTTATGATTGGAGTTGAATGATCTCAGCACCCATTATTCCATACCAAATCA
CTATGAATTATCAAAGGGAAACCTGCAAGATTAAATCGACTGTCATGGGCTTTGTTTTAGGAATTCTGTG
ATGTTTTATTTTTTACCTTTATAATCGCTTGGTCATGATATGAATCAAAAAATAAATTTTTATTGGTTTA
CCTCCTATTTTTTATGAAGAGAATGAATCTTCTTATCGAAGGATCAGAAGAAAAGGGGTTTGAATTTCCT
GCGGGAATGATTTTGAATGTGGTTAATATCATGAAGAATAAAGAGATAGTCTATACCCAAATAAAAATAG
ATGTTCTCATAAGGAAGCCATCGAAGGCTTTCCATCCTTGCATTTATCTGTATAATTTTATATTGTGCTT
TACAATTCCTAATTTCTTTTTTTTGGTAAACAAATAGAGAATATTTATTTAGATTATGGCTTCTAAACGA
TATTGTTCTCAGGGTGCCTCCACGCTCTTTGGGCCACACACTCTCAGTGCCTACATTCAGGAGTTCAAGA
AGCTTGCAGCTGCTCTTACCAGTGGCCAGTCGGTCGAGCCCGGTCCACAACCCCCTGATCTTCTGGATAA
ACAAATAAGCTTACTTACACCAGTTGTAATGGATGCAACCCCACCTGGTGTCCATTTCGGTGATTGCAGC
TCTGATGTACCCCTGAACTCCACTTTCAAAAGAGGAGATACAGTCAAAGTAGTTTTCTGGTCAGCTTGCC
CAAGGAATGACCTGATGACAGAAGGCACCTTTTCCCTTGTGGAGATCCTCCAAGGAAAGGACAACTGGTT
CCCAGCATATGACGATGATGATTTCTGCCTTCGCTTCAAGTGGTCGAGACCTTCAAAACTGAGTACTCGT
AGTCATGCAACCATGGAATGGAGGATCCCGCAGTCTGCCAACCCTGGTGTCTATAGAATGAAACATTTTG
GAGCTGCAAAGAGCCTTTTAGGCTCTATTCGCCATTTCACCGGTTCATCAAGTGCTTTTGTTGTGACATG
AAAAACATACCGTGCATGTACATTTACCTACTTTACTTGTGTGCTTGTATTCATTTTTTTTATGTATAAT
ACTTGCACGACAGATAGCTAGCATGTGCAGCTCCTGCTGAACTTTTGAATTCCTGTTTTTGTAATGCCTG
GAGAAAAAGACATGGTTTACCACTACTCCTTTCTTAAGCTGGTGACCAGGTAAATTTATTATTTTGTTCA
TGCTGCAATCACCACTCATCCTCTGGCTTTAAATAAATAAATAAATAAAAATAACCATGGATCGAATTAA
AATATTTTAATTTGAAAATATGACTATTAAAACCTACAAATATGATAGTTTATTTGTTGTTATTTTGGAA
TAAAAAATCCACTTCAAAATTCTCTCTAAAATGGATTTATTTATGTCTTTTTTTTAATAAATATAAACTA
GAGGGAAAAGAAATCGTTGTATATTTGTTATTATTGAGTGCAGGTCGTTCATCAACTATGAATAAACCAT
TTATCAGTAACTTACTAAGTAGGAGGCAAGTCGTTCCATGAGAAGAAAGCCTTGACAGATCTTTGCACTA
ACCGACACGTCGTTTCGTAATTTCCGTTTCGAATCTCGGAAAGTAGAGTGGAAAAGAAGAACCCAAAGTA
GCTACTGTGTTGAGTTACATAATACTCTATTACCAGAGTGAAGAATCAATCATCCCTAATTACTAGATTC
TCTTGGAAATTGAATTACAGATTTCTTCTAATTTTCCAATCTCAGCTGCTGTGCAGGAAGGCTCCAATCT
CTCTCTCTAGTTGTACACTACAGGAGTTGAGAAAGCAAAAAGAAGAATCGAGGAGGAGAA
Sequence ID: 36
Sequence Length: 3095
Sequence Type: DNA
Organism: Poplar sp.
AAGGAGGAACCTGTACTTAGAAATATTTCTAAAAGGCGGAGAGGACGGGTCCATATCGTCGTATATATTT
TAAAAAAATTCTAATGGGCAGAGGAAACGGATCCATGTCGATGCCTATATTTAGAAAAATTTCTAATTGG
AGGAGGGAAACCCAATGAGAAAACCTCTGTTGACGCCCATACTTGGAAAAACTTATAAGGGTAAAGGGGA
TGGACCCATGTTGATGCCTGTACTTATAAAAATTTCTAAGGGGCGAAGGGAAACCCAATGAGGGTAATGC
TATTTCACTAGGGAGACTTTTGGTCCATCCCTTGGAGAGTGACTCAGAGGAACCCATATTGACACTCGTA
CTTGGAGGAATTTATAAAGGGTAGAGGTGACAGATCCATGTTTACGCCCATAATTAGAAAAAATTTTAAT
GGGTGGAGGAGATATAAGGGAAACCCAAAAAGGGTAATGCTATTGCACCGAGGAGACCTTTGGTCTATCT
ATTGGAGAATGGTTAGGGGAACCCGTGTCGACTCTCATACTTAGAAAAAATTCTAAGGGGTGGAGGTAAA
CCTAAAGAAGGTGATGCTATTGCACTGGGGAGACCTCTAGTCCATCTCTTAGAGAGTGGCTTAGGGGAAC
CAATATCGACACCTATACTTGGTAAAATCTCTAAGTGATGGAGAGGACAAACTCATATTTACTCCCATAC
TTAGAAAAATTTATAAGGGTTAGAGGGGATAGACTTATAGAGATTTGTGCAATCGTACTATGGAGATTTT
GGATACATCTATTCCATTGAGAGTTATGCAATCACACTAGAGAGGCTTCAGATCCATCTATTTTAAAGAG
AGTTATGTTATTGTACTAGGAAGGTTTCAGATCCATCCCTTTTAAAAAGAGTTATGCTATTGCACTAGGG
AGGTTTTAGATTCATCCCTTCCAAGAAAAGTTGTGCAATCACACTATGGTGGTTAAATTAATCTCTTTCA
AGGTAAATTGTGCAATCGCACTAGGAAGACCATGGTGACTCATTTCGAGATAAATTGTATAATCGCACTA
AGAAGACTAAGTCAATCCCATCCTCGGAAGGTTGTGGGGCTATTGCCCAAGTCGATCCCTTTCTACTATT
TATTTAAGAGTTGCTAAGTTTTGATGGAGCAGATGTGCCAACAATTATAGGGAGGTCCGTGACATGTCCC
ATTTTAGAAGTGTCTTGATTATCTGTTTTTGATGGTGATTAAAGATGTCAATAAACCACTTTTGGAAGAG
CAATCAATGATTTTTTTTATATCAAGTTTCCACAAACGACGTCAATTGATGAAGAGAGTGTTTGGGAATG
CAGTGCAAACGGCGTTCCTAAAAATTTTGAAATTGTTTTTTGCTTAAAATAATTTTTTTTATATTTTCAG
ATCGTTTTGATGTGCTGATGTCAAAAATAATTTTAAAAAATAAAAAAACTTCATTTTGATGCATCTCTAA
GTGAAAAGCACTTTAAACAGTCACCGTTACCACAATCCCAAACATGCCCAAAGTTCTAAAAAATCAAAGT
AGCTTAAGAACAAAGCTTATATGTTGGATAATCGGTTAATCTCCTGGTTCTAACAATATGATCAATTCTC
CCTGGTCAATGTGGCTGTTGCTTAATATTTGCAAAATATCACCTTTGATAGATTTGAGGACTCTCAAATG
TTTTAATAGGTATCTTTTTAGAGAGTGAAATGATATTTTAGATAGAGATGCTATGAAAATATACATGCAA
AAAAAAAAATAGAGATGGTAAACCCTTTACCTAAGTGGTTTAATAAAGAATAAATAGTCTTGAACCTTGT
CATGTTGCTTGAATGGAGGGGTTGAAATGTTATTTTCTTCTCATAACATTAATGAAAGATAAATACCCCA
TATACAACATTAATGTTGATGAAAATATGGTAGGTCTTAAATAACTTTATACACCCAAGGGTGTGGGAGA
ATATGATTTAAATTGTATTGGGTCAGTAAAAAGATCCTTTATGATAAATAAAGAATTTTTAGATGCTAAG
CTTTTCTACAGGGTTGAACCCGTTATGGCTAAACTCTTCGCTAAAACTAATTCTATTGGGGTTGAACTCT
TTTCTAAGGTTAAGCTAATGATGTTGATATTTTTTTTTTTTTTAACTGAGTTCATGAGGGCTGAACTCTT
CTTCAAGACAGAGCCCATGAGGGCTTGGGCTTGTCCCAAACATGTTGCCTTAGCCCGTGTATCTTTGGGC
ATTCGAGAGGGTTTTTTAAGACTATTAAATTTTAATTTATCAGTTATAAAAGTTATAAAAACCGAAGCAC
CATGATTGATGTTTTGCATTTGCTGGAGTTGATTCTAAATAGTTGGCAGATCCACTGTCGTCACCATCAG
AATGGAGTGCAAATTCAATGTCTTTTATATATATAAAAACTTGTTGGGCATGCAAATCCAATGGATAGAA
CTCAGAATGCCAGGATTCAATTGCTTGCATGAGATGGTGCTACCATTGGATTGGCTTTAATTTAGTTCCA
TTTGTTTTGCAACATTTTTTATTCTAATATCAGACAACAAAATTTGGGCATTCATGGTAAAATGGGTTTA
ATTTTCCTCCTCCTCCTTTTTTTTTTCTTTCTAAGTAATATTTTGAAAATGCACCAAAAATAAAAAGACA
GCGCATTGGTCCCCTAAATATGGACCGGAAAAAGCCACTTACAGGTCACAGCCGAACAGGCCCAGGTAAG
GTCCACTCACGTCCAGGCTGATCCACCCTTCCTGCCTTCCTCCCTTCCTTCCAATTGAATCAAAGTCTAG
CCTCGAGCGGCAAGATTTGAAAATATAATTGAAAGGGACGTAAAACCTAACTATAGTTAATACATAAACA
ATGAAAATACACCACGTCAGCAATCATTGCCGTATGTTGCCCTTTCTTAACCAACCCCGGTTAAAAAATC
CCCAAATGTCTGTCCTATAAATAACCATCACCCAAGTCCATCTCCTGCAAAATCTTCCCCAGAAGGCGTC
TTGATAAACGCTTTCGTTCCAAATCTCCTTCTTTCTTCCATCGGATCTACTTTTGTTGTTTTCTTTCGTC
TCTTAATCAGCGAAA
Sequence ID: 37
Sequence Length: 3000
Sequence Type: DNA
Organism: Poplar sp.
CATTCCACGGAGATATCTGAAATCGTTCTCTTTATAGTTTTTGATGTCCGCCATGGCGTTGACAGGAATC
GGCATCGCTTCCTCCGCCAATTTCTACATCATTGGCTAAGGACAAAGACCGCAAATTCCATTCTTGCAGT
GCCTGTGCTGTGCTGCAAAGCAACCCCAAAACCCTCAACCTCAACTCTCCTGGGAGGCGTGCGAAGCATG
GTGGAAGAGTCACAAAGAAGATCTGTCAACAACGGCAAAACTAGTCCATCCTTCAATATCTTAAAAACTC
GACGACTTCTTTTTCAGAGCGTGATCTGCATGCACTATCATTCTCAATATCTTGCTTCTCAGGATACATA
CAGAATCCTGATCAAATGCACAGGCACAGCCATGTCAATCACTGTCACCCACACCTGAAAAAAGAGACCT
TCTGATAATTCACTCCCAGGCACAGCCATGTCAATCACTGAAACTCACACCAAAAACGCATCCTGAAACT
CATTCTGCATTTTCACTCTTCTAATTACCTAATTCTCTTCTCCTTCCTCTATCTTCTAGAAGCTTGTCCG
ATAAAGATTATGATCCTCGAAATTGGAGCCGTTCGATCAACTCTGAATTCGATGACGATAACGACGACGA
ACAGGGGGGGGGGATAGGAGTTTGGATCTGTTGCTTAAGTTTGTTTCAAATGTGTTCAAGAAGGTATCGA
AACGTGCTACAAAGGCCGTCCGATCGGTTTTGGCCGTTCCTTTATCCACTAAACTGGTAATTATTGTGAA
TTGGTTCTCATTGAATTAATTTGATGAAAATGATGGCGAATTGTTTTTCTTTTCGGATTAATTTCGTAGG
TGGAGTTTTCGGTGAATGGAGTTTTATTGCTGGCGTTTTTGTGGGTTCTGAAAGCTTTTCTCGAGGCAAT
ACTTGGTATGTTATACTTGATTTTCTTTTTTACTTTATACATTTCATTCAAAGCTCTGAGTAGAATGATT
CAATTGTTTGTTATGCTACTTCAGTGTGCATGTCTTTTGCAGTTCTGTAAGTCCTGGGTCTGTTCGTTCC
CAAAATCATCGAAAATGTAGAAAAATGTCCATTTATCTTTTATACAGTGCACAGTGTCTGTGCACTTGTA
CATCCAGAGGAGGGGATTTCTCTCACTCTATGGCATTGGATGTTAGCTGTTCTATGCCCTTCTGGCATAG
TAGTTGCTCATGCTGGTGGAACTTCTTGAACCAAGCTTGGGTTTCTACACTTTGAAATGTGAAGGACTCA
ACAGTTGTAACCACTGTTGTTGAACCATGCTTTAAAATTAGGTCATTAATGCTTAGCAAGTCCGGCCTTG
AGTTCATGTGCATGAAAATATTGCTGTAAGCCAACTGTGTCATAATATACTTTACTAGATACAGCCGTAG
TGATTTGTGAGCCTTACGGACATTTATTTCTATGTCCTCGTAGTGTATGATTGCTGCTCCCTTGCTTTCC
TCACAAAACACTCGGTAGTCTCTGAAAACATGACCATACTTGTTGGAATCAACGGCTTGAGATTTGTAAA
ATTCGAGATGGATTATTGCCTGGATATATAGTTGTGATATTATGCCGCTCATCTCTCTGTGTTGGTGTTT
GAAATGCAGGTGGTTTTGCACCCTTGGAAGTGTGGTGCTTGCAAGCATCTTACTTATACGTGGGATGTGG
TCTGAGGTAATCTCAGGAAGGATGAACTCGATGACCCCCGTGTATGGACTGGCACGCAGCCTGCAGCTTG
ATTTTTCATTCTTAGAGCTGTAAATGTACTTGTATTTATGACATTCTTCAAGAATAAAAGCATCCTCGTA
ATCGAACTTCCCAAATCAAGGATGATGAAAAGATTTCATCTGAAGGTTTCACACGTCAAAATTGCTGCAT
TGGAAATAATCTGCTCATGTGAGAAAGGGTTCCCCGCATAAAGATCCAGATCAGTGAATCCTTGTAGAAA
CTGGAGGAGATCAAATTGAACGTACGGGGGCAATTTCCCCTTGTATATCTTCAATAGATAGACACTAGCC
ATGTACTCATTAATATGAATGATAAAAGATTGTAGTTATAGAGCTTTTATCCTTCAACAATTTAGGCGCA
ACCGTTGAGCTCGTTGGGTATTTTAGGTGCTTCTTCAGGGTTGCGGTGTAACTTGTTTTAGGATTTTTTT
TAAAAAAATTTATATATATTTTAGATTATTTTGATGTAATGATGTTATAAATAATTTTTAAAAAATAAAA
AAAATAATTTTTAAAAAATATTTATTAAAATAATATTTTAATATATTTATAAACAAAAATATTTTAAAAA
ATAACTGAATATAGACTCGCAAACCCTATGCACAATGAAGTAAAAGCTCAATGTTTTTATTTTTATTTAC
TATTTTTTTAGCATACAAATCCAATGGTTAGAACCTATAATGTCAGGATTCAATTGAACATTTAAGAAAA
TAGTTTCCCGAGATGATGCCAGTATAGGATTCGCTGCATCTGTTTGGCAATATTTTTTGTCAGACAGCTA
AATTTGGGCATAAATGGTAACATGATTGTTTTTCCTTTATTTTATTTTTTTTAAATGCACCTAAAATTAA
AAAGACAGCCATTGGGAAAGGCCACCACTAACTACCAAACAGGCCCAGGTAAGGTTCACGTGTCCAGGTC
CACCCTATCCACCTTCCTTCCAATTGAATCGAAGGACTTATTAGCTGGCCACGAGCGGCAAAAATTTTAA
AAACAATTGAAAAGACAGAAAACCTAACTTTGGTTAATAAGACTCAATAACAACCGACCACGTCATCGCT
GATGGCCGTTTGTTGCCCTATCTTAACCAACCCCGGTTAAAAATCCCCAAATATCTCTGATATAAATCAC
CACCCAAATCCCCCTGTCATTTCCATCTCTTACAGAGTGATCTCTAGAAGGCGTCTTCATAAACGTCTTC
GTTCCTAATCTCCTTCTTCAACAATCAGATCTATCTTTGTTGTTTCTTTAATCAGCGAAA
Sequence ID: 38
Sequence Length: 3122
Sequence Type: DNA
Organism: Poplar sp.
ATTTTGTTCTTCATTTAGAAAATTAGTGGTTAATATTTTCTAATTTGTTATAGAAAGATGGGATTTAGAG
AAAAAGGGACAGAGTAAAATAAAAATAAAAATAGAGTAACTAAGTGGCTATATAAAAATTGGCATGAAAA
ATTATTATAGTTTACATATTGTTCTATGGAAACATTTTCAATCCTTTTAGTTTTTTAAAATTTAACTTTT
GGTTCAAAATTTTATTTTGCTTATTTTTTAGTCTCTAGATTTAAGAGATGAGAGATACTTACTCGTATTA
ATTGAGGAGAAAAGAAATGCCGGTTTGTGTTGATTCCAACCATTAAAATAATCTATTTTGATGTCAATAA
GTTCATTTTAAATAAGAGGAGTTCCAATAAGACGTCACTTAGCCTTCATCTTCACTAAGATGGTAGATTT
GGGCTCATGAAGTGTTTTTGAATATGTGTTGATTCTTGTTTTTAAAACTAATTTGTTTTTAATTTGAGCC
TATATATGAGTTTATTTAAGTTTTTAAGATGTTTATTATGTGTTTTTGAGTAAAAATAATTCAATAATAT
GTTTTTCAGATAAAAATATTATTGGTCTGACTTTTTAACCACTTGAGGTTAGGTGTAGTTGAATTAGAGG
CTTAAATATTGTTTTTGGATTAAAACAATTCAAGTTGAATTTTTAATTAAATCGCACATGCTGATTCAAT
TGAAATTACATTAAGAGTCATCATACGGTAAATCTGGTGTTAGTTTGGTGTTATAGTGCAAATTACTTTT
TAATTAAAAGATAGCAATGCTTCCAGCATGGTGGGCTCGTTTTTCAAATCGAAAGCTGCTTCTTCTTCTT
TCTTTGTTTTTTTTTTTTAATCTTGTTTTTCTAATTTCATAAAAACCAATCATTATTTCGCAGGTCAGGT
AGTTAAATTTGTTTGGCTAATTGATCCAGAAACCTCCGGAAAGTCAAACTCAAATAAACTGCTGACCTTT
TTATTTATTTTTATTTTTTGAATTCTAATTCGTCGGACTATCTGGTCAAGATAATCCACCTCTCATGCGA
ATACTTCTTAGAGTGCCATCCATTATACCCTGTTAAGTTGCCGGTGGTTGCACATGTTTGACCACCCTCC
CTCCCCTAATTTTCACGGTGGAAAGGGGCTTGTTTGGGCTTGTTTGAAATTATAATAATAGTGATTATTT
AAAGTGTTTTTTATTTAAAAATATATTAAAATAATTGTTTTTATTTTTTAAAAATTATTTTTAACATGAA
AACAACATGAAAACATAAAAAAAATTGTTTTCATCCTTTTTAAAAATATTTTTTTTTCTATTTTTTCTAT
TTTTTTTCAATATTATTATATAGTTTTCTTATTTTTATTTTTCTATAGTATTATTAGGTTTTTCTGTTTT
TTTTTTTAATTTAAAGGAAATAATTTTTTTTATATTCAATATTATTAGAAATTTCTAATTTTTTCTATAT
AAAGGATTTTAAAATTGTAATAACATTTTGACAAGAAATTTAATGAATAAAAATTAAATATTCTAGATAT
CTCTTCACAGTTATGACATTCTTGGTTTTAATTTATAATAAATCGCATTATCATTAACCCTCGGCTAAAT
TATCTATTTATTTATGACCATGGAAACACAAGTGCGTGTGTATTTGGGGAGGTGTGGGTTTAAAGCCTGC
AATATAATTGAAGAAAAAATTTAAGAATTTTTCCGCGTTGATGAAACCCTGATTGAAGGTTGGAGCATGC
CTCAATAGGCAGACGGGCGAAACTTAGAAACCAGGAATAAACGTGAAACACGGGATTCACACGAATTTGG
AAATCCACGCTTGTAAAGAAAACCAAACCGCATAATTTTATTTCCTATTTGTTTTCGCGTCTTGTTTTTA
AAAAATTTAAATTTTATTTTATTTTTTTTTCTTTAAATTAATATTTTTTTGATAATTTTAGATCATTTTA
ATATGCTGATATCAAAAATAAATTTTAAAAAATAAAAAAAATATATTATTTTAATATATTTCTAAATAAA
AAACACTTCAAAAAACAATTATAACCATATTTTCAAACAAGTACTATTAAAAAAGTGATGGACAAGAGAA
ATCAAGGGGTCGCGGATGCGCTTAAGCTATAGTGAATGACAACTAGTCTAAAGTTAAAACTTAGACCTCC
TCGCGTAAATTTTATATTTATATTTTTAATATTAATACATTAAAATAATTAAAAAATAATTTAAAAATCA
TTAATTCATACAAAATTTTTAAAGCATATTAAAAAGAGAATAAACGGCAAAAACAAACCTACGCTAATTG
TGAAATAAAAGATTAATCTATGCACACGGTATCGTTTTACTTCACTGGTCGGTGTAATAATTTCTCTAAC
CTTATGACCCAACAATTCACTATTTTGAAACCCTTGTTGTTATTTTTTTATCAACCTTTTTCTTAATCTC
CATTTCACTCATTCCAGTTGCCTGGACAGTGGACATGGTGGCGGTGCCTCTTGATCTTTTCTAGTTGGGC
CACATGAATACACTTCAAGGGATTTGAAACTAGGCCTAATCGATTGAAACGTAGAATCCACTCTCTAATT
GAGAGGACGGCCCACCCTCCTGGGCGACGTGCCCTCTCATCCACCAGGACCACCACCATCATGCCTTCTC
TGCTCCTTCCTCACGCCTCCCAACAGAATGACATTATTACCCTCCATCCCAACTATAGACCGGCAGTGGC
ACAACTGCAATTTCCTACAACCCAAGACGATCCCCAAAACTAAATTCAAAAATCAAAATGGAGCGGGCAA
CTAACCATGGTTAAAATAACGATTCGGCCAACCTGGCAAAATCAAGAATTAGGTGGCTTGGGAAACGGCA
TCATTGGCATGCACCTAATTTGACCCGTGGTTAAACTAACCCTGGTTAGCTAAACCACACACTCCCTCCG
TCCCCTAATTTCTCTCCCTCTGAAAGTATATAAACCCCATACTCACAGACCTAAAAAGCTCACCCCTGAA
ATTTCATAGGCGTCTTGATAAACGCCACCCTCCCTCAGCATCAATTCCAATTGTCTTTGCTTTCGATTTT
CTCTTCTTTTAATATCTGTTGATCTTTGTGCTTTGAGAGAAA
Sequence ID: 39
Sequence Length: 3000
Sequence Type: DNA
Organism: Poplar sp.
ACAAATCAGTATTTCAAATCCCACATTTTAAATTACCAAAAAAAAGAAGAATACCCTTAAATTTTGAAAA
AAATTAAAAATTATACTTAGCAATATACCCAAATGTTTTCAAAGTATTTCAACTTTTTTTTTTATAGTAA
AAAATATTCTTGAATTAATTTATATATCAATAAATTTAAATTATAAATACACTTCTATTTTTGTTAAAAA
AACATTTGGAGATTCTAAACCTAATTTAAAGACTGTGTTGGCCTAATGCAGCAATTATTATATAAGATTG
ATGTTGTTTGGTATTGCGTTTTAAAGGGGAATTGAGTAAATTAAAAAAAAAATTTGTTTAAAATTATATT
TTTCTATGTTTTTGAATTGTTTTGATGTATTGATATTAAAAATAATTTTTAAAAATTAAAAAATATATTA
TTTTAATATATTTTTAAATAAAAAAATACTTTAAAAAATAATTTTAATAATTTGACGTAATAAGAAATGA
CTTGTGTCTGATTGATGCTTAGCAGACAATAATAGCATTGGGAGAAGCTGAATCTGCTATATATATATTG
GATTAAATTGGCAATCAGGTGGTCTCCAGAAGGTGATCTCGAAAACAGATTTTTTGGTGCATCTTTTTCT
CCAATGCCCCCTAGAATCTAGTTAAGCTTGTTGAATTTTGGTGAGCTGACGGTTAAAATATTGAAACCCA
CAATTTCAATTTTATATTTTCACAAAAAAATAATCCTTGAAATATTATAAACACAATCTAAGCACAAAAA
AATCATTTATAAACAATAAATTAACGCATGAATCAAATTAGTTGTTTTAGATCTAAAAATTAAAATTAAC
ATCTCTCCCTATTTTAGGGATGAAATAGTTCAATTTTGATATTTATAAATTCGTTATGATGAGAGCTATC
ATTTGCTGATGTTATAGCCTAATTCCTTTAACAAAAATTTCTTTCTCCTTTCTCTGTTATTTTTATTTAT
TTTTTATTTTTTATGATTTTAAAATTTTAACGTTTTTATTTAATCCAAAAAAACACATGGATTAACCGGC
CTAGCTTTTTTTAAGGTTATAAAATCTGGATAACCTCATTAAAAAGAAACAAAAAAATCATAAAGCTGAT
TTTTAAAAATAAACAACATTGGAAGATAAAATTAAAAAAAATAAGTAAAAAAAAATACCCCTTCAAGGAG
GCATCACAACGTTTGGTTGTTGAACGGTGCTTTCATGCACCACCCGAAAGACGCAGGCTTTCTCACCTAC
TAGCACGTTTGTTACACGCACTAATCATTTTTATTTATATATTATATTTTAAATTAATTAAAATACATAA
TTACCTCTTAGTCAAATCTATTATAACAAAAAACCTAAATGAAAACACGAAAACGCTTTAAAACGCCAGT
TAAGGAAATTTTGCTTTTAAAAGTAAGCTAATTATTTTATTCTACTTTTTGGGGGGGGGTAAACTTGGGC
TAATCTTCCAAACCCACAGCTCGTGAAATCCTAAATCTAGGTTCAATAAAGAAACTTAATTTCATACCAA
TTTAATATTGAATGATGAAATCGAAAAATAAAACCATATTAATCTAAAAGTTCTATTAAAGTAAAACAAA
AATAGCAATTAAAATAATAAAAATCAAATTTAATAGGAAAAAAAATTAAGGATGAAATCGTAAAAAAAAA
TTCATTCTTAAAAATTACCTTAAATAAAATAAATAACAATCAAAAGAATAAGAAAAAAATATGACAGATG
AAAAAAATTAAAAGAGGATGAAATTGAAAAATAATTTCAATTTTATAAATTATTTTAAATAAAAAATACT
AATAAAAAGAATAGAAATCAAATTTGAAAAAAAAACAAATTGAATAGCTGTTTTGAAAATTTAGAAGGTC
AATGTGATATTTAAGGTGGAGAGAGAAAAGAAGAAGAAGAAAAAAAGGCCATGAACACCAAATCAAAGAT
GTTATATCACACGCATTGTCGAGGAATGGAGGGGCCACCTAAAACAACCAGACGATCGTGAGGTCTCCCA
ACATGCCAACATTTTTTTTTAATAATACTTCATATTTATTAAAATACATAATTTCCCCTAAGTCAAATTG
ATTATAATAAAAAAACAAAAAAAGTTTTTGTATGCTAATTTACTTTTGTTTTTGGCATTTAAAAGTAATT
GTGTCATTTTACATGTTAAAAATAAAAAAAAGTGTCATATTACCATGTTAAAAATATAAAAATACTAAAA
AGACTATGTATTAAAGGTATAATTCTTTTTTTATTTTTAAATGGAATTTCGTAATTATACTATGCTATAA
AATATGAAAAGAATAAAAAAAATCCTTCGATGAAATCTATTATATATATATATATATTGTTTTTAAGAGT
ATTTTATTATGCTAATAAAATATTAACAAATTTATTTGTCATTAATTAATATGATAATATCTAATATATC
ATTTGAAAAAAATCATAATACTCTCTGTAACAAGCTTGTTGGGTTTTTACAAAAATAGCGAAAGAATCAT
TCGTGAAGTGTAAAACTCTATGACTTTTAGATTTTTAAAAAATAAATAAATTAAATGGGCTATATGAGAT
GTCCCCAGCACTATTGTAGCCCTGGTCCTATCTGGTGGACCGCCAGCTTGAGTTGCAATAAAAGACCATG
TTAATTCTACGAGCCCATAACACCAGTCCAAATACAGCCATGTCTATTAAACACTGATCCGCCCATTTTA
CTCCATAGTTATTGGGCGCCTTCCAGAAATTCAAATTTCAAATTTATTCTCAACGGTTACTGCCAGCTGG
ATCCCAGCAGCAGCAGCAACCATTTCAAATTTCAAAAACACCCATCTTCTTCTTAAAAGCCGTTTCAAAC
GGCCCTTTCTCTTCGTATATATATATTCTTAATTTCCGAACCCTAAATCACAATTCTCTTTGCTCTGTTT
AGCACTTAGCAGTTGGCCGAATCTTGTTTAATACAATTTCTCTATTTCCCACAACCAAAA
Sequence ID: 40
Sequence Length: 3131
Sequence Type: DNA
Organism: Poplar sp.
TTCTGGAATTTGATAAATTATTGAACACATATCACAGGCATCCTACTCTGTCAAGTGACATTAGGAATCT
TGGTTGTCTTCCATGTGTTTGTGCGTTTGTCTAAGTAAATTCTTGTTTTACCTGTTACTTCTGGTTGAGG
TCTTATTTCTATTATCAGAGCCGGCAAGAGCCCCGTAACCACTTGAAAAATAGTTCAAGATATTCACTTA
ATACTAACAAAATAAAAATTGTCCAAGACTCACATGTCACCATGCATAAAAAAAAAAAATTAAATATTTT
AAAATACCTCATTAATAAATTTACCCAAGACCCCATTCACCTTAAATCAAGTCTGCATCGTATATATATC
AAATTGATGGTAACTAATTGCATGCAAAAGCTTAATGAAAAGGCAATACATCTAGGGTGGTCTGCCATCA
CTTGACAGCGAAAAAGGAAAGAAGCTCTCGTTTATTAATTTTACTACCAGAGAGATATGTAGAGGAGGGG
ACAAATAAAATGAACTGTCCATGTTCTGTCATTTTCCTCGTCTACTACAAACTTGGACACACAGGGTTGT
TGTAATGCCAGCAAATTCTTGGGACTTAGATGCCCTGTCGCTCTTCCATTTCTGATTGTGATCAATTCCC
ATCTGCAGATTTAAACAACTGCAGATTTAAACATATAGTATACTCTTTGTTCTTGGGATTTTGTCGTTCC
TGATATGAATGGGAGGGGAGAAGAACAACCTTTTCTCTTTACACTCATCAGATTCATTGAAAGAGATGTA
GGCAGAAGGGTGTTTTTCCTTGTAATTTAGCCGGCATGTTGTTTTGCTACTTGTAACTATATGTGTTACA
TCTTTAATAAAAGCAGCTCTGTTGGTCCAACCCCCAGCAAGAAACCAAATCTAGCTCTTGATTTCGAAGC
TTCATCTCCAGATTTGCAGGTGACCATGATCATGATCATGGCTGCTGCATTAACCTTGTGAGATAATGTG
GTTGCTGTTATCATCAGCTGATGATGTATCCCTTTAATTTCTCACTTATTCTATCACCTAATTACTCGTA
AAGGAATCAAGATTAGGGGTATGGTAGTTTGGATAATTAACATACATCACAGACTTACATTGTTCTTACT
GAAACTTTCTATTGAAATATTAATTACAAAAAAACTAATAACCAAGAGTATAAGATTTTGAGGTTAGGCT
GGTTAATAAATTGATTTATTATATTTTATATTCAAATTTAAAAGTTAAAAGGGATTAGAAATACAAGATT
TCATTAGTATCCTGATATGAACGATATATTTGGTATTGTTTGATTATTGTCTTAATATTACGAAGATTGT
GAATATAAAAAATATATAATTTTATATACTTTTTATTTTGAGAGTATAAAAATTCACTTAAAAGTTATTA
TAAAAACAAATTTATTTTATATTTTTATATTTTATATTTATCATATGATACTAACAAAATACAAAAATCA
ATTATGAAAAATCTATTTTTATCGTAAAAAAAAAAGTCGGAAAATACCGGAAACATTAAAACCGAAAAAC
AAAATTATAAGATTCAAAAAATGCAATTAGAAATCTAGCAACTTTCTTTCGATTGTGAGATGTTGGATAC
CACACAACAACTCTTGTAGTAGAAGATTCAAAGATTAAAATACAAGATAAATCATTGTGGAGTAAGATAC
AGAAGAGGAAAACCATAAAAAAACCTAAAAGCAAACAGAAGCAACAAACAAGATCATATGAACAGCAAGT
GAAACAAAAGCTCATGATCTCTTGAAATGAACACCGCACCCACCTCCCAAACGGTCCTCCATTTTTCTCC
CATGCCCAATATTACCCTAAGAAACTTCCCTTATCTCCCATTTTTGTCCCTTAATCCCAACAAAATTCTA
GGTCAGATGGGCCGTCATGGGTAGGCCGGCAAGGTCGGAGTGGGTCCCGAGTAAGGCTGGCGCCAAATGG
CATGGCCCCCTTTTTTTCTTTCTTTAATCGGATTTCAAATATTTTATCAAATTTCATTTGTTTCTACTTC
AAGTCATCTTGCCCGATTCAAAAATAAAATAACAAAAAAGGAAAGTGGAAGAAATATATTTTGGTACTAT
GAATTAAAAGTATTTTCACTTTCATCCCATTTAAATTCTCCGACATTAACTTTAGCTTCTAAAAAAGTCT
ACTTCAGTCCCTAACCATTAAATAATAGTTAATGGAAACCAGAGGAATATCCATTATTTCCTTATATTCC
TTTCATTTTTTCAAGTTTTCTTTCTAAATTTCTTTTTATGTATCAGAATATCTTATTTTGACCTGAAAAA
ATAATATTGATTCTTATGATGGATTTTTAGTAAAGATATAATTTTTTTAATTTTAGTGATTATTTAAAAA
TAGTGTTTTTAACACATTGTCATGTTTATTAAAACTAAATCATTAAATTCAAGTTGAATTATCATGATTT
AACACATAATAAAAATAAAAACAATTTAACTTTTATCATTTGACATCTCTCGTCTAATCTTAAATCTTGT
AATCAAAATCAATTATTATTCATGTTTTCATAAAAATATAATCAGATTCAAATAGGACAAATAAATGTTA
TAAATACAGAAGAACATAAATATATTTGGTTAGGAAAACAAAAATATAAAATAAATTTATATATAAGATA
TTTTTATTATTTCAAAACATGATGAACAGATTGTTTCAATAGTTTTTTTGTTTTATAACAATGTATGATG
AAGAATAAAATATTTTTAAATTTATAATGATTAAAAGTATAATTTAAATAATTTTAAAGTAAATTTTTAT
TATTTTGAAATATAAAAAAAGACCATCTTAATCATTTATGCGAACAGTAATTAAACGACATCTAATGGAC
TAATTAAAATTTTAAACAATTAAAAACGGAATGAAATGTTTCGGAATCTATAGGGATCGAAGTATAGTTT
ACCCAAAAAAGCAACTCTGCATAGAGATACTAACCTAATAAAAATCTTTTTTCATCATTTGCAGCCTGCC
TTTCATTTTTTTCGATCTATAACAGCAACGTCGAAGAAACAAAGAGAAAGAGAGAGCGTTTTTGTGTGTC
TGAACAGAGAGAAAGAAAGAAAATTTGAGTGTTTAGAGTGAAGGAAAGAAG
Sequence ID: 41
Sequence Length: 3021
Sequence Type: DNA
Organism: Poplar sp.
TTTTTTCATGCAAAATTGAGTACCAACTCAATATCAGAATTTTTTTTATATAATGTAAAAACTTGATAGA
AAACAATTCAAAATAAATTACTAAGTTAAATCACAAACCAACATAATTGTGCTTTTAAAAAAAATTTAAT
TTTTTTCTTTAAATTAAGTGCTTTTTTATGTTTTTTAATCGTTTTGATGTGTTGATGTCAGAAATAAATT
TTAAAAAATAAAAGTATCTCCGAGGAGAAATGTTATTTTGAATAGCTAAATTGATAAAATAGCATTTTGA
ATAGTATAAATATGTCTTTTTTTTACCATGGATACTCTAATAAGAAAAAGCTATTTGAAAAGCTAATTGT
ATTAGAGTAGGAGAAATAAACATTTTATTTTTTTTAATGAGTTTGATGTTATTTTTTTTCTACGTGACTA
TATTTAATTGGTTTATTTTATTGTTGGCTCCACTTTATTGGTTTTAGCTTTTTTGAGAGATATGTAGGAA
TAACATTTGTTGAAGAGAAAAGACATTTTGACATTTTGTTTAGCACTCGGAATATATAAAATAAGCATGA
AAGAGCCAAAATAATATATATATTTTTTTAATTTTTGGCTTTTCATTTAGCACATCTTTTGGAGTTGTTT
TAAAAATTATTATTTTGATGTATTTTTTAAAAATAATTTTTATCATTATTTTAAATAAACCCGAACACAT
GTTTTAACCATCCATCAGTAAGTATGATGGCCCCAAAATAATATATATATAAATTACTTGGCAAATGGCA
CAACATATATATATATATATATATATATATATATATATATATATATATATATATATATATATATATATAA
TAGAAAAATGTAAGTGGATTAGTTCAATTATTGATTGAAGTGTGTCTTAACTTTGACATGTTAGAATAAA
GCAAAGAAATGAAAACGTGTGTGTTATCTATTTAAGAGAAGAGGTTCAATGGATAAAGTAAAATAATTTA
CGTAAGGTATTGTTTGCTTTTTGTATTTTAAAAATGTTTTTTTTTTAAATTGAATGTTTTTTATTGTTTT
CTTATTTTAAATTAATTATTTTTTAATGTTTTCATATTATTTTAATATCCTGATGTTAAAAATAATTTTT
TTAAATAAAAAATATTATTTTAATATATTTTTAAGTAAAAAACACTTTAAAAACAATTATTATCATACTT
TTAAACATCAAATACAAAACTAGTCATCTCTATTTTTGAAATATTTTTGAAATTTTTATCGTGTAGTTTA
AAGTTGAAACCTTGTAATAACAAAAAAATAAAAAATTCATAGTTTTAATTGCTTAATTGTTCCTAGGGAG
ATAACAAGCCCACAAATTGCTTTAATAATATGGATTATTTTTTCAATTAACATAGTAAATTAAAAATATT
ATGACAATATAATTGAAAATAATAATAAAATAAATGTGTTTATACATGTTATAATTAACAAACATGATAT
GTATAATTTATCACTATTTAAAATAGCAACATTTAATAAAAAATATTAAATGAGTAAAAGAAAGAAGAGA
TGAGAGAAGAGAAAGAAAAAAAGAAAAAGACCTTCAAGATATACCGAATTTCTGATTACAATATCATCAA
AAAAATCTCAACGAGACAAATCGAATGACACCAAAAAAGATTACCAACAATGATTTGAGTGGGTCACACT
TACCGTCCAAAGGTGAGAGGTGACTCTGTTGTCTTTAGCGGGAAATGTGACCCACTCAAATCATCGTTGA
TGAACTTTTTTGGTGTCGATGAATTTGTCTCGTCAAAATCTTTTTGAGAGTACCGATGATATTATAATCG
AAGCTTTAATGTGTCTTCATAATCTTTTTATTTATTTTTTTTTATCTCTCAAACTTATTTAATAGCAACA
AGTTATAAACATCATTTTTTTTTAAACTATAACATGTATAAAATGTGCTATTTTTTTTTATTTGTTCACC
AATAATAAAAAAAAAAAAAGTGCTAATAGCCAATAATAGTGTCTGAATTAATGCATCAATAAAGTTATAC
GACCTTAAACGTACTCCATTAAATTTATCCTTCGATTTTCTTCATACACTTTGGTGTTATTCTCCTGGAT
ATTTGCACAAATTCACCTTGTGTGAAATCAAGCACATTAAAGATTTAAATCGGATTGCTTGAATTATTGA
TAAGTTGCATACATAGCCAGATGAACTGAAATTTTGGAGCTTTTTAAAAGTCTGCCACCTTTCAAATGAT
AAAAATATGCACGTGTTGTATTTTTTTGCATATAAGTTTTTTTATATAAAAAAAAAAACAGTTTTATTTT
TTTAATTAAAAAAATTATTTATTCTTTTGTAAAATTAAAAAATAATCAAGAGAAAAAAAACTTATCTAGA
CTGGGATTCTTAATTGCAAGTGTTTCTTATCGAGTAGGATTTGATTGTTTTTTTTTAATTTATTTTAAAA
ACGTTGGTTAAATAGAAAATCTATTTAAAAAAACAATTAATCTTTTCTACCATTAAAAAAATGTTTAAGT
TTTTTAGAAACTCTATTTAACTTAATTTTATTTAAAATAAATAAATAAATATTTTTTTATTAATATGAAT
ATCCGGATTAGTTTAAAATATTTTTTTATTAATATGAATATCCGGATCAGTTTACGCGTACTTTAACAAT
GTTATTATTTAAAATATTTTATTTAAAAATAAATTATTATCAATTTTTTTTTAAAAGGTGCTGGTCTTTT
TTAAAAAAACTTAAAAAATTGTAAGAAATTAATTATGCAATCCCATCATATATTTTGAGCATGACGTGAG
CCACAGTAATCGACGGGCAGCAGCCGAGCTAGCAAAAGAAGCCAGCGCCATACAGTTGGCGCCATATAAC
TGGCAGTTAAGAATCTTCTTTAATTAATAAATTAACTAACTTTTTATTCAGCAACACAAAAGAAAAGAAA
AGAGAAATCTCTTCATTTCTCCTTTCTTTCATCTGCATTATAATCATTGTTTAGAGAGAGAAGAAGAGGA
AGGAAGGAATT
Sequence ID: 42
Sequence Length: 3118
Sequence Type: DNA
Organism: Poplar sp.
TCACTCATTTCCATATCTTGTAGGCTAGATTTGTCTGGAGCGATGTCTATACTGAACTTGTATTGTTTGA
CAGAAGTGGCAACCAAATCTTTCCACCTTTTAGTCTTAAAATTGTCAAGCTTTATACTAAGGCCATCTTG
GAAAAAATAGATAAGTAGTTTCTCATTGTGGATAACTTAAGCCATTGTATTGTAGTAAGCCTTTAGGTGA
GTCAGTGGGCATTGCATTTCAGTGTATTCAGCAAATTCAGGCGCTTGAAAGTCTTTTGAAACTGCAACAT
TTAACACTAAACACATTTTAGCTGCTTTCACTAGGTTATATAGATTAGACTCTTCAATCATCTTCATTTT
TTCATCCAATTCTGCCTATTTTTTGTAAGCCTTTTCTATAAGATTCTTTTATAGAGCTTTTTCTCTAGTT
AAACCTACCACAGGAGGTAGCCTTATGGGGTAAATAGGTGGGAAGCATAACAGCTGGATATGAGGGTTAT
TGTTGTTTGAGGAATTAGAGCTTCCACAAACCTTATTCTTAATGCTTACTTAAGTAGGCTTGTTAGCCTT
TCCATGTCTCCCTTAAAGCTCTCTATTTCCTTCTGATGTTCAGATTCCAAGCATGCACGCTCTTTGTTCT
TCTTTAGCTTCTTTTTGGATCTTGTGTTAAAAATTCACTTTAAAAGGGGACCTATATTTCATTAATCTGA
TGATGACAATTAAAACTAAAAATATGTTTGCATATGAATGCTTATGCATGCTTATGAGAATGTATATTGG
GTATAGATTAGCCCTTCCGAGCGAATCAAACATCTGAAAGCAAAAAAGGTCTAAAAAAATGAATATGAAT
AGGTTTTCTACATGGTCTAAAAAAAGATCTTAGATTTGTCTAGTGATAGTCTTTGTAAAGATAGTATAGG
GACGTTGCGAGGATTGGTTAAGGTATGTATGTCCATCATTACTAAGCAAGCACTTAAATATGAGTTGAGT
TTTCATGACTCTAAACCCTGACCCATAGTTATTGTTGGATAATATCGGGGCAAACCAATATTTTTGTTAT
GTTTATTTACATAACATAGTTAATTAATACATGTTGTCGTCATAATTTTATAGAGGTTCGATAGAAGTCA
TGGATGATCGTTCATGGATGTATCGGGACTCACCCCAAGGATTGCGGGGGATGGATTATTGTAATGGGGT
TCAGGGTTTTATTAATTTCGCAACATCTATCCCGAGAAATTTTACTGATGGCGGTATTAAGTGTCCATGC
AGGAAGTGTAAAATAAAAAGTTTCTGCATCAAGATGTTGTAACGATGCATCTTCTAACCAAAGGGTTCAT
GGAGGATTACCTGTGTTGGTATGTACACGGAGAACTATTTGTTCCTAATGAGAGCATGGTAGAAAGGTGG
TTGGGTCAACTTTTAGTGCTAGCAACATGCATGAAGTTGGAAATGACAGCAGTAATCATTACAGGAATAT
GGTTATGGATGCAATGAGAATGAGTGAAGGTAATGTCAGTAAATGTCCAATCGTAAAAGAAGAATCTAAT
GCAGATGCAGTAAGGTTTTTTAACCTGTTGAAAGATTATGACAAACCATTATGAGATGGCTGCACGAACC
ACAATAAATTATCGACCATAGCTCAAGTATTTACCATCAAATCAGATCACGGGTTGAGTGAAGGGGCTAT
AACTTCTTGTTGTTGCCATTATTGTTCAGGGGAAATGCTGATGTTTTGGGTAGGGTGTGGCCGTCGTGGT
GGTGTTTTGGGCAGCATAGAAAAAAAGGTGTTTAGGGTCTTCAATGGTTGTTCCCAGGAACTAAACCCCA
TGCAAGAAATTTTTGGAAGGAAAAATCCAATCAAATCTCCAGGCAATATAAGAGAAAAGGATGAGGTTGG
TGGCTAAGGTTTTTTTTGGTTTTTTTTTTTTTAAATTTCACGATAAAATTTTAATATTTAATAAAAAACT
TTTTAGTTATTAATTAAAATATAATAATAATCCTATTTAATTTTTTGATGATTTATTTTTACGCGCCAGC
TCGAAGATGAATGTGTCATAAACTTCAAGATTCAAAAGAACAGAGATAAAGTGGCCTTACTGTTCCACAA
TCAACATTTGATTTGGTGCCTAAAGTTAGAAATGAAAGTCTTGCTTACTTTTGAAGACCCAGTGAACTTT
GGGCCACATGCCGTCGAAGATCGGGGGTTAAAAATCCAAAAGCTATACTCTCTGAGCTGGGTAGCTAGAG
GGTCTTCTCATAAGAACATGCACCACCAACTCCCGTGATTCATGAACAAACATTGCTCCACAATTAAGAC
GCAGTTCAATGGGTGGTTGTCTTGATGGTCACGGGCAGAAAACTTGTTGGTTCTTTATCTCTTACCGTAT
TGCAGAGTCTCTGCTGCATCATGGCAAAGGACTGGTTTCGAAGCAGCAATGTTGACAGAGGACACAGGGC
AGGAGCACAGAAATATATTCTATCTGGCCCCAAAAGTCTTGTTTCTGCTTCAACATATCTTTTCTCCCCT
GAACCTTCCATTTATTTGTGTGTAAATGAAAGATATTTTGTAAATTTTCTTCGTACGTTTTGTGTTATCG
TCCTCATCGAGCACAAAAAAATGAAAGATTGAAAATGGATTGTTTGAACGTAGTCAAATGAATAAAAAAA
GAAGCAAAACCACCTCAAATGTCTCTTCACTGCTCAATTTAGATCTAAAAAAAGCAAGCCAACTCATCCG
TCCAGACAACCAGAATCTTTAAACAGAGTCCATCAGTTTCCTCTTAAGAGTTTAACTAAAAATTAACTTT
ATAAAAACTAATTTTTGACAAGGACGTGGGCCATGGTAACCGACAACAGCCGAGCAAGCCCAAAAAACCA
GCACCATAGAGCTGGCGCCAAGCGGCGGACCCCATTTGGCGCCACATAAGTGGCAGTTAACAATAAAACA
GCAACCACGAATTGATAAATTAACTAACTTTACCTTTATAACATTCATAAGAGGAGAAATCTCTTCTTTC
TCTCTAATTTACATCTCCATTGTTGCATAGAAACTCTCTGAGCTTCTTGGAGAGATAAATATATTGCTTA
AAGAGAGAGAGAGAGAGAGAGAGAGGAAGAAATCAATT
Sequence ID: 43
Sequence Length: 3149
Sequence Type: DNA
Organism: Poplar sp.
TAATTAATTGTGGCAGGATCACAGTTCGCAGGCGCTCGGATCTGTCTCTGGGCCTTAATGTTGGTGAACA
GTGAACACCCAAAAAGAATGTTGCCAGTGGGCTCTTTAAGGCCCAGTACGAGAAAAAAAGCATCGCAATG
TTTTTTGTTGATGGTCCCAGGAGCCCGTTGTCTGCAGAATGGGCTCTTTAATAAAGCTCTCGATGATCAT
TCCAGATAGATTTTTCACGTCAAAGAGTTGCTTTCTTGGTTAAAATTAAGGAGTTTGCTTCTTTTATTTT
AATTTTCCGGGTTCAAATCTTGAAGTTAATATTCAAAAAAAAAATTATTACGCAATCTTATACATTTAAA
AAAGAAAGAGAATCATAATACTTAGACAGAGAACAGTGAAGACAAATGATATATGGATACAAGAGGGGAC
AAATTACATACATGTCCATGTTCTGTTTGTCCTCATCCGCCATGTACTGTACTGGATATATATATATATA
TATATATATATATACAGACACACACACACACACACACACACTTGTTGTTGTTGATTAATGCCAGCAAATT
TATGGGACCTTGATGATGCCCTGCTGTTTTTGTTTTTCTTTCTTCATTTTCTGGTCGAAACCAATTTAAT
TTCCATTTACAACAAATTTAAAAGCATGTATTTACTCTCTGTCATTCTATATATAAATGAGAGTGGGAGA
AGAACAGCATTCTCTCTGTTTTCTCTGTAAACTTGCCAAACTCTCATAAGTAGCCACCCTCTTCCTTTAC
TGCTATGCTATATTAACTCAGTAAAGCATGCAAAAAATGATTAATTATTATGGTGAAAATTACCTATATT
AGATTTTGATAATTGAGGTTGAGTTTTATTTTATAGATCTAAGGCTTGAATTCATAATACGAGCGCGGCT
ACAATTGCACATATATTTTTAAATTAAAAATATTATTTTTGCGCACATGTTTTCAAATGAACACTAAGTC
TAATAATGAAAAACTTATTTTCAATTTTTTTTATGTTCTTGTAATTTAATTAATGCATATTGCACTAGTG
TTTATTAATGGTAATTAACTATGAGTCATTGTGCAAGCTACTAATTAGGTGAGAATAGATGGAGAGGAGA
TGGTGATAAGAACACTTGATTGGATGCATCTTTGATTTTGTGGCAGAAATGATAGTTTTTAAGAAGAATT
GAATTTTTTTTATTAAGTTTTTTTTTAATGTTTTTATATTGTTTTAACATGGTTATATTAAAAATAAATT
TTAAAAAATAAAAAAAATATTATTTTAATATATTTCTAAAAAAAACAATTTAAATACCAAGCAGACAATG
ATTACACTCCCTCTCTCTGGTGGCTATTTTTGAAGGGACTGTTTGAAGGAATATTGTAGCCATGTGAAGG
CCACCTGAGTGAAGGCATTAATAAATGTGGTAAGCAACTCTGTTGGTCCAACGCCCGGCAAGAAAACAGA
GCCACCTCTGGACTTCGAAGCTTCATCTCAAGACTTGCATGTGATCATCTCGGTGATTAATTGATCTGAA
ATCTGATCTGAACCTTTAGTTAAATCGGATTGAAATTAAATCAATTTTAATTAAATAAAAAATAAAACAG
CATTGTTTTATTTTTATTTTTTTAAAATCTTGAAAGAATTCCGTCTTGAATTGATTTTAATTTATCTATC
AAACTAATAACTCTGATCTTGGGCCGAGCAAGGGATGACCCGAGCAGCACGTGGGAACTTAGTTTTATAC
AATAGTAAGTGGAATTAAGAGAAAGAATGAGTTCTTTGTGTACCGCAGTTTTGGGTCAATCCCCAAGAGA
GTGTTTTTATTGTATATTGGAAGTAGACAAAAAAAGAGAAAAAGGGATCGACTACTTCCATGTTAATTAG
AGGGAAAAGAATCAAAGATTAATGCGGCTGCGGCAGTTGAGATTTTCAAATTTGAACCTCAAGATTTATC
TCAATTTGCTCAACCATGGTCGGCAAAGTTTGAACCAACCTAGGTCACAATTCCTAGGCTATGATATTAA
AATCCAGGTCATGAAATATACTCAAATCAATTAAAATTATTTTACAAGTTAAAATGATATTCATTTGAAA
TCAACTTTGAATTGTTTTGTTTTTTTCTCTATATTATATGAAAAACTACATCTTGAATCCACGGACATAC
CCTGATTTAGACAGTGTTCAAGAATGATGTTGAAATTATATTTTTTAAAAATTTGAAATTTTTTTATGTT
TTCAGATCGTTTTGATGTGCTGATATTAAAAATAATTTTTTTTATTTTTTTTAATTTTAATATATTTTTA
AACAAAAATAAACTTTAAATATTATAATTTTAAACACTATATTAATCGATAAATGTGTCACAAACATCTT
AGCAATAAATATTTGTATATGGTATTTTACAACAGCTCACAGCATCTGCAAATGCACATGACACCCACCT
CCCGAGCGGTCCTCAATTTTCCACCTATGTCCAATATTACCCTAAGGAACTTCTCTTATCTCCCATTTTC
TCCATTAATCCCAACAAAATCCACGGTCAATAAGACAGCAAGTTTGGAGTGGGTCCCAAGAAAGGCTGGC
GCCATATGGTGGGGACCATTTTTATTTTTTTTATCGGATTCCAAATCTTCTATCGAATTTCATTTATGTC
TCCTTCAAATCTTGCTGTCCAATTAAAAAAAAATAAACAGAGAGAAAAGTGGATGGCTATAGTTGTAGTC
CCTATAAATTCAAAAATGTTTCATTCTTGACCCTTTGTTTTAGATTCTCCAACATTAACCATAGTTTGTT
CTTGTTTTCCGTGTACATAATGCAAAGTTACTTATTGTTGATTTCCATTAGCTATCAGCAACCAGGGGCT
ACATTGAAACATATTGGAAACAATGAGACTAACATCATCGACACGTTTTTTAAAGTACATAAACCGGAAT
GAAATGTTCTGAATCTATAGAGGTGGAAATATAGTTTACCCAAGAAGGCAACTTTGTATAGAGATAGAGA
GAGACTGAGATCCAAGAAAAAATCTTGCATCCTTCTCTCGATCTATAACAACGTCAAAGAAACACAGATA
GAGAGACTTTGAGTGTCCGAACAGAGAGAGAGAGAGGAGACTGTGAGTGCTTAGAGTCAAGGAAAAAAG
Sequence ID: 44
Sequence Length: 3007
Sequence Type: DNA
Organism: Poplar sp.
TAATATATTAACTTGGTTAAACCGACCCTAGTTTTTTTAGTTGTTTTGATAATTGTTCTCTAAACACACT
TATTCTACTGCAGGATGGCTTAGAAAGGAGAGGCTTTAAGGAAGTTGGTTTCTTGAATGCAGTGTCTGAT
GTGGTTAGCACAGGTAATTTAGAAGTAATTATGCATTTATTTTTGCATGTTTCCCCAGTAATTTGGCTAT
GTTAACCTGCATAAGTTGGTTTCTTAATGGTCGATGATTGATAATTTTATGGCAGGTGTAACACCAGCAG
AAAAGCTTTTGGAACTTTACCATGGAAAGTGGGGGCGGTCTATAGATCCTGTTTTTGAAGAGCTACTTTA
CTAAGGTGAATTAGATTTTTGTAGCACTGCAGCAGTGTCCTACTGTGTAATTCTCTCTCTCTCTCTCTCT
CTCTGGGTTTAAACCTTGTTTATGTTCTGAACTACTTTCTTCAACTGATACTGATAGTTTGTCTGTCTTT
TGCCGAATAAATTTCAATGTAAATCCTTGGTACGAGGATTCTCACAAATCTCTCATCGAATAACACCCAG
ACAGTTTGGAACCAGTGAACAATTTTTTAGCTTCCATAGCAATAACAAGCCGATCATGTGCTTTTCACAA
GCATTCATTGTAACGTTTCCCTCCTTGTGTCACGCATCTCATTCTCATCATCCCTAATGAAAATGGTTAA
CCCGATTTGGTATCTAATTCTATTTTCACAAACTAACCAGGTTTTCATTGATCACAGTATGCAAATGGAT
TATGAGGTTGGTGAAGTGTGTAACAGAAATATGAAAGGAGGATCTAAACTTAAATGCTCCCTATTTTCAG
TTCAAACACTGTTACATGGCAAACAAAATGTCAGTACTCATTTTAAGAATGATGGTAGCTAGCTTCATTT
CATAAAAAGTCTGTTTTCAGTAACATCCTACATTTCCCCACAATAGTAGGACCTTCAATGCTCTTCTAGC
AAGTATTTAATTGTCAATTGCTTGATTTGCCTTTTGTTTCCCTAAAACCTTATGTGATCTTGTCCACCAA
CCCTATCTCACCAGGTCACCCTCCACGTCCGAGCCATGTGGGCTGCCAGCACCCACCAGGTACCGTGTGC
TTCCCATACCCGCTAGAGATACATACCCCACCATATTACCATATAATATGGTAGCTATCCATGCCACTGT
TTTGTTTAGTCAAATGTATCTGGCTGGAGTGAGAGGTGTGAGATTGCTGCCCGGGGGGGGGGGGCGCTTC
CTAACATAAGAGGGTGGTAATTTAGATGGGGACTGGCCATCATCCCTACCACTCTTGAGTAGGTGTTGAA
TGGCTTGAAAACGATGCTATAACAGCAAATTCTTAGACACATATCCATGAACATCATCAAATCTTATCTC
GTGCTACCTTTTTGTCTGTTAGTGAAGTAAAATGCTAGTATGAAGTTTTGAGTCAAAATTCAGTCAGCTA
TCATTTTCCCATTCAATTTAGCCTGCAGCAGATGATGATGATGATGGAAGTATACTGAGATCTGTTGAAT
GTTTATTATGTATTTAGGTTTAATAGATTATATTAGAAGCAAACTACCAAATCACACTTTTCTCTTTTGG
CTCAATGGGATTTTTTTGAACCTCAGACTACACAATGGGATTATAAAGTATCCATATTTTCATATAATCT
CTTCAGGTGATGTGAAATCATTTATCATATTATAGGGAGGACTTAGCATAGGATCCCAACATCAAGAAGC
CAACTAGTCCTTGCGGAAGGTCTGGAAGGAGGGCCAGAAAGAATACTTGTTTAAGAAGAGAGAGACAGAC
CGAAAACAGGACGGTCTCATGCCCTCCAATAATTGAAGCTTCCTAGCTCTCACCCTTGTGACACCACCCT
TAGGTTTTACATCGCAAATTAGGCACCTAATTTGATCTTAAATCACTAATGCGATCTTACACCATGATAT
TTGTGGCATAACCACCTTATCAGTCTGAACATTACCAGAGTACCCTCCCTATATGTCTGTCATCTCATTT
CTGTCCGAGTGACCAAGTTGTCTGAAAAAATCTCGAAATAATGAAGAGGTTTCTATTGACTGCCAAGTTT
TATTGATATGATGCTAGACCGTAAATTTCTACCAGGTAAAAATTTGCTGGCAATGTGGTTACCTTACCTG
GTGCAATCAAGGTACTAGGAAGAAGATTTTAATTTCCTATATATTTGCACGATCAGTATGTTCTCTATAA
TATGACAAGAACTCAGTAGTTATTTAATATTTTTGCTTAAGAAAACAGTAAAACTCTACAAGCCACCAAG
GAATTAGAGATGTTCATCAAAAGAAAGACAAGGCGTTAAAGAGTGAGTAAAGGGATCAGCATTCATGAAA
TGTTCTCGTTAATACACCATTCCAGAAATGGTGGATTGAAGTAAGAAGGAAGTTGATAATAAAATAATAG
AATTCCATCCAATCATTTTGGAGGAGAGCAAGCTCAAGGCAGAGAGAGAGAGAGAGAAATGGAAGTAAGA
AGGAAGAAGGAAGTTGATAATAAAATAACAGAATTCCATCCAATCATTTTGGAGGAGAGCAAGCTCAAGG
CAGAGAGAGAGAGAGAGATGGGGCAGTTTTGGAATCTTAAATGACAGACAGACAGAGAGAAAGAAAATCA
ATCATACTGGAGGTTGGGTTGGCTATTTTAGATTGAGTGAAGCCAATGTATGATTGCAACTTCTTGTTAT
TATTATTATATCCATGTCCAGCTCACAACTCTTGTTGGAGCTTGACATGTTCCCCTCCTCCTAACGGAGA
CACATCGTATTATTCATTAAATTTTCTCTATATTTCACACTTTCTGACACGGAAGATATCAAACTCACCT
TCTTAAAATCCTACCCCTCTTGCACCTACAAAAGCCCTGCGACTTTACATTCTTCGTCACTCTCTCCCCT
CCAGTCCCCTCTCTATATTTCTTCTCTTAACATTTTCATTTTTTTTTATTAAGACTAACCAATCAAG
Sequence ID: 45
Sequence Length: 3000
Sequence Type: DNA
Organism: Poplar sp.
AAAATGGGCCAGGGGGTTGCTCCTTTTTCTTTTTCTTCTATTTTTATTTTTTTTTATATTAATGATTTTT
TCTCTATTTTTTTTTAATTTATATATAAATTAATATCCCTTATCTCTTTTATTTTGTTTGAAGAGCCCTT
TTTAAACGATAATTATTTTTTTAGTGATGCATTTAATTTCGAAGAGTTTTTGTGATTCATCTATACTTTT
TTTATTTTTTATATGAATGAATTTTATTTTTGGAAATAAAAAAAATTATTTTTAGATAATATTTCTAATA
TGTGCAGCTTTGCATGATGTTTTTTCCCATTTTATTTTTTTCGATCAATTTAATTTGTGTGTCTATTTCT
ATTATTGTTTGCTTAAATAAAAAAATTAGCAAACACAACCAATTAAATGTCTTGTTTTTCGAGATTAAAT
AATTCGATCAACATTTTTCTTGATCAATTTAATTTGTGTGTCTATTTCTATCATTATTTGCTTAAATAAA
AAAATTATTTTAATAAAAAAATTAGCAAACAAAACCGGTTAAATGTCCTGTCTTTCGATATTAAATATCT
TGATCTATATTAGTGTCTTGATTTTTCAAGTTTTATTTTGAGGTATCGTCAATGACTTTTTTATTTATTT
ATTAACACACATAGTTTTTGAATTATTTGATTACATGCATACATAAAATTCTTAATTTGAGCTTAAATTT
TTCAAACTACAGTATGAAATCTTTTTATAAAAATAAAAATATCTGAGTCTCAACATTGCTGCGCTAGTCC
CATAACTAGTGTATCCTAAATCCTGTCCAAGTTTTTGAAATCCAGATAGAGGATGATGCCAATTACTCAT
TCCCTTTCAACGGGGCGCTGATTTAAGATTTGAATTCTTTAAAATTAGTCAAAGAAAGAACACAAACACA
CCCTCCCCCCCCTAATTGCACAGTCCATACTCCATCTTTTAAGACTAATTCATTCTTCCAATGTAGGGAA
GGATGCCGGCAAAAAAGTGCATAAACCCAAGTGGCTTTGCAATTTCAAGTTTAATTTATACACTATCCCC
CCCCATCTCGGTGTTTAATTCGTGTCGACACTCTTGGCTGAAAACGTCGTCTAAAATCAAGGAAGCGAGA
AAACAATCTCGGAACCAAACAGTGCCATTTCCCCATCGGACCGCCCTGTAGAGAGTCTGCTTTTGGATAA
AAAATCACTCTCACATTCAGAGACCTAAGCGATGGCAGCATGCTTGGCTCTCAATCCAAGCAAGGAATCA
GTTTTCATTAATGGCGAGGATTAGTGGAGGAGGGAGGGTGTGTTAATTAAGCTGCTTTAGGTACAGAGTC
TGGATCCCGTGATTATGATGTTGAATCCATAGGAGATAAGGAGGAGAGTAGCTGACAGTTCACATGGGGA
CATCCTCGCCTCATGTGGCCCTTCTAGCAAAGCTCTTTCCCACCTTTTTTTTTTTTGTCCTTTTTTGCCA
TTTGAAGAAAGATGCTTCTCTCCTCTAATCCCGTTCCGTTACTTAAACTTAACAGTGATTCTTAATCAAT
GCAATTGGCAACAGTGCCTTCTTCTGATAATAAAATACGTGAAAGGCCCATCGCGCTCCCAAGAGCTCAT
CATTCAACTTTTTACCCTGTTGATATCTTCGTATCAAAATTATTAAAAATAAATAAAAATATTATTTTAG
ATAAAAATTATTTTTTTTTCAAAAAAACGATTGATAACCACTTTTCTATTCCCCCATTTGTAAATGTGAT
TCCATTTGAGTCCCCAAAAGTTAAAAATTAAGCAACGCTGTCCTCGAATTTGTGTCCTCTATTTGCTATC
ATCCTACCTTATTTCTTGCCTTCTCGTATCTTGTTCTCGATAATAAAGTTTTAAAATTCGGCCCAGCTTG
ATCGGTTGATCCAGGACCCGGCCGACTTAGGGCTGTAATCAAGCCGGGTTGAAGAAAAAATAAAGGAAAA
AAAAACCATGTATGACCCCGCAAGATCCGGTCAAAAACCCGGTTACAACTCGTTGACTTTTTTTTTACTA
AAACAACGTCATTTTGATTTTTTTTTTTAAAATTGACCCGGGCGACTCGGTGACCCAGTCAAAACCCAGA
ACCCGGGCCTTGAACCCGGCCGGGTCTAAAAAGTAAAAACTATGCCAGATAGAGCGGCCTAGGCACCTAA
TTGCAATCATCTGCACGTTGGGACACCAGTTTGTAATTTTCCCAAGAATAGAATAAGCACAAGATCTGTC
GTTTCAACAGGTCATTATTAGTCCATTAAACCCATTTTTCAAATATCAAATATCATTGCCCTGTCAATTC
AAAACCAACATCTACACCGTTGAACATCACGATCCAACGGCATCCAACGGACTAAAACAAAAGGAGAGGG
AAACCACGACAGCCGTCCGATCCACACACCTCCACCCCTATTTTCCTCCTTTATATAACTACTCATTCAC
TCCATTTTCAAACCAAATTATTACTTCCCTCCTCCATCACTCCCTTTGATCGAGCCTCTCCAACGCTCGA
TCTCTGAAAACCTCACCGGAGACACCGCATATTTAAATCCGGTCACCGATTTCGTAACGGTGAGTTCTCG
ATTTCTAGATCTGATCTCTTCTTCCGTTATCGAACCTGTCTTGATGCCGAGAAAACATAAGAAAACAAGT
TAATATCTATTTTGTTGATGAATAATTATTGAATCTTATTTTTCTGAGTGAATTACTTTGTTTATGCAGT
CTCATTGCACTTTCTGTTTTTTTGCATTTTCTCTGCAGCCAAACAGGCTGGATCTGTAATTATTCTGTTT
AGACTTTTTGTTAATCTTCGATCAAAAGCTTTATTTAGTCACTTAATTAATTATTTATTAAGATATAATT
GTCTAGTGAAATTTACTTACGTTTTAGATCTGTTCTCCATGTTTTAAATTATAATTTCTCTTATGCTTTA
CCTAGTTTTTCTCTTACCATTAATTTAATCATATTTTTTCTCAACTTGCAGAGATAAAAA
Sequence ID: 46
Sequence Length: 3000
Sequence Type: DNA
Organism: Poplar sp.
ATATACGGTTTTAAGAATACTTAGTTAATTAATACTGTTTAAAGTTATCCATAATGTTTTTTTTATCTCT
TGTTTTTTTTTATTTGCAATTTAATTATTTTATATTGAGTTGATTTGAGATTAGAATCAATTTTTTTTTT
AACTCTATACAAGGCTCTCACGGTGTTAATAGTAGATTTCAATGTTGAGTTAATATTTAATTTAACAAAA
TAAAATTTAATTTTATTAATTTAAACTAATTAAAATTTTAAAAACCTAATTTAAAACTGGAAAAAATATT
CTAAATTTTTTTTTATAAAAAATAATATCTTTGTCTTAGTTTTCTAATTCAATCTTTGACTATAATTAAA
AAAATTATCATAATTTATTAACATATATAACTCTTAATTTATTTTACTATAAATGTTTTAGTTTGTAGTT
AAAAAAATTTTAAATACCATAAAAAAAACATTAACAGCACTCACGTTTTTTTTTACGCCGAGAAAAAAAA
AAAAAACTCTATCATGTGGTGTAACACGTGGAATCAGACTAATTTAAATAATGAGGAGTGAAAGAAAATA
ATTGTCAATAAAGTTAAAAATCACATTTATAAATGATAAAATTGTTATTTTTGTTAGGTGTTACTTCCAC
GTTATCTACGAAATTGGAGAGAAAGGTTTACGTGGGCCATTGCAGAGTTTGTTTTTCCCTTCATTTTCTT
TCCCCACACTCTCTTTTATATTCCCCAACAACTGAAAATATCTTGAAAATTGCATTTCCTCCCTCCTCAT
TTTCTTGCCCTCCACTTTCCACCCTTTCCAGACACTCTTGACTATTGATTCGATGAGAGAGGAAAAAAAC
ACGTGATCAAATAATAGGGATGGCAACGGTTATCTATAGATTAGATTCTTTATATGTATACATTTTCTAT
TATTTATAGGATAAAAAACTTAGATATTTATATAATATTCATTGAGTTTCAAGCATCAACATGGACATAC
ATTGCCTTATTTATTATAAAAAAAAAATAACAGTTTATATATATAAGCTGTATATAAAATATAAAAGGTT
ACCTTTTAAAAACAGACCATAAATACCATACCTTTAAAAAACAAAAATTGTTAAACTTGCATCCAATAGT
TTTTTTGTCTTTTCAATTTTTTTTTTAATGATAAATAAGCTCATAGAGTTTACTTGGTCTTTTACTTAAA
ATGAATCTTATAAAAACAAATAAAATTTGTTATTGTGTTCAATATACCATTAAACGAGGGGGTAGTGCAT
GTGACGTCATTCACTTTCAATCATTGACTTTCAGGATGATGTAGAAAACATCTCGCCATCTTTTATATAT
TGGTGCCAGGCATGTATATGTTGGTGTGGTGGATGAACTCCAATGAACTTTTATTTTTCTTGTCATTGTC
AAAATATAAAAGTGTTGTTTAATTTGTTTTTTTTTTTAAAATTTGATCATCATTTTTTTTTAATATTTAT
TTTATTTTGTTTTGTTTTTTTATCTTTTTTAATTAATTGTTTTATTCCATTTCGCTCCTCATCATTTAGT
TTCATTTAATTTTTATATAAGATTCGATCCTTATTTTTCTGATTGCTATTTTTTTTTTAATTGATTTTTT
TCAATCTCTTCCTGCATCATTTGATTTTAAATTATTTTTATATAAAATCTAACCCCTATTTTTTTTAACT
ATTATTTAGTTTGTTTTCATCATTTTTTTATTGTGTTTTTTTTTAATTTCATCCCTCAATATTTTGATGA
TTTAAATTTTTGCTTTGTTATTTTTTAGGGTTTGTTTTTTATAGGATTAGTCTTGGGCTCAAGACCCGAG
TCATGGGTTTTAGAAGTTAACACATGTTGATTTTTTTTTTGTCCTTTATTTTTTTAAAATTTTATTTTTC
AAGATTTAATATATAGGGAACTAGTCTTCGTGTTTTTTTTGTCATTTTCATGGAGTTATCCCAATCATGT
GATCATTATCATAATATTAGCGAGTTAACTTGAATTTTTTTTGTTGTTTTTTTTAACTATTTACTTCTAA
ATCTTGTCCTTTAACATTGAATTATTTGGTAATTAAATTTCATAATTTTTTTTAGCATGCTTTTAATAAG
ATAACCTCGATATTATAACCAGATATAGATTCATGCTATCCTGAATGTGCTGAAGTTATATTTTTCTATT
ATTTTTTACATTTATTATCATTATATTTTTTTTATTTATATTATTTAAATTGATAAAATTCATTAAACAT
AAGTGAATAACTCAAGTCTTACTCGCAGCTTAGCACGGACCGTATCCAGTCACTATCAAACGGCATCTAA
CGGGCCAAAAGAAAAAGAGAGGGAAACAATCATAGCCGTCCGATCACACAGCCTGGCCCCGACCTTCTTC
TTATATACGCGCGGATTTTACCATCTCTGCACTCCACTCTTCACCCGCTCCCTCTCACTTGCTAGATCGA
GGCTAACTCTCACTCGATCGCCGGAATCCTCGCCGGAAACGCCTCCCTGTCACACTGGTTTCCGATTTCT
CAACGGTGAGTTTTCATTTCCAGATCTGATTCCTCTCTCATTTCAAGTCCGTCTGGATACCGAGAAAGAA
CATAGAGAAACCGCAAAAAGCTGAATTCATTCTGTTTTATCTTTCCGATTTGAACTTGTGGATTCAATGC
TTTGTTTTCTTAGTTGTCGCGGCTTTCATTTGCTTGCATTTTCTCATAAACCAAACAGGCTGGATCGGTG
ACAACACATTGTTTTAAGCTACTAGTGTGTTTATCTCCGCTATTTTTAGTAATTTAATTGTTCTTTGCCA
ACAGTATCTTCTCCTTCGAGGATGTGAAATTTGTTTCGAATTTCGCCTGCTGTTTTTTTTTTTTTTTTAA
CTCAAAATCGTACTTTTAAATAGAGTCTCGGATCTGATGTCTCTGTGCAAAATTATTATTTTTTTCGAGA
GTGCTTAATATTTTTTATCCGGCTTGCCTTTTTTGTTTCTGCAGAAATATAACATAAAAA
Sequence ID: 47
Sequence Length: 3000
Sequence Type: DNA
Organism: Poplar sp.
CAATATTAGCACTAATAATTTAATATAAGTGAATATTTAGTGACAAAAAATATTAAATATATTATAGAAT
TTTGTAAAAAATTTAGATAATATTTACATTAATTACAAGTCCATTAGAAAGCCCATCAGAAAACCCATTA
GAAAGCCCAAAAAGCCTAAAAAAGCCCAAAAAAACAATAATACAGGTTTTCCGGTTTTTCTGTTAAAAAA
ACCGAACCGAACCGAAACCGGTCGGTTTGAACCGGTTCCGGTTCGAATTTTTCAGTTTTTCAAAATTTAA
TTTTTATTAGTTTTTTAGATAAAAACCGAACCGAACTGAAATTACTCACACCTACTCCAGCGTCTTCTTT
GTTTGTTTTCTTGGTTACTGTTCTCTCGTGTTCTTGTTTGTGTTGCTGGATTCGCTAATTAATGACCACA
AGGATTCCACATTCGGTCCAAGACAATAATCAATCAAATTATATTCACTTAATTCCTCTTCGCTTTATGT
CTTAATCATCGAATGATCATGAACGAGGGCCGTACACGTGGAATTATTGACCTAAAAATGAAGGAACAGA
TGAAGTCCAAGTTCAATTTAATTATTCCCTAATAACTGCTCTGCCTGTCTCCATGATGTGAAATCTTCAA
TTGGTAAAATGAAATAATAGCATGCATAGAGTAAAAGGAAAACATAGAGGAAAAATAGAGCTTAAAATAA
AAGGGAATAAGTTGAAAACCGAGGTGAAGAAGAGTGTAAAATATTCTATCATAATATATCCAGAAATACT
ATTCCTTTGGTTTTTTTTTTTTTTAATAAAGGATTTGGAATCAAAACTGATATAAAACTATCAAAATTAA
TTTATGCGATGATCAACCTTAATTACCCGTCACAAACCATAATTAGACTTGATATCTAGGTTGAAGAAAC
TTTTCAGACTCTTTATTTATACCTTGGACAAACTAATAGGCTGCCAAAATATTTTTTATGCATTAAAGAA
TCATAAACTCAAACCTAAACCCGAAAATAGATTGATAAAAAATAATTTTTTTATCGTCTAAAAATTATTT
TATGATAAACCCAAATCTAAACCTAAGCCCAAACCCAGGCTTAGGCTCGAGCACAGACCAAACATGTGTG
TGTGAGACTTTAAGTCCAAAACCCATTTTACTTAGGTTCTATTTTCTCTAAAAGCTTTGGTGTAATTCTT
TATATTCCCATTATATAAACTATAAAAAAATCTTGTTTCTTTTCTATGTGAGATAAAGGGTTTTGAATTT
TTCAAATTGAACCTTTCTTAGTGAAATATTATTGAATTTACTATGCCAAATGGTGAACGTAATGTCTTTA
ACTATTCAACCTTTTATATAAACTAAGATAATAATATTCATATAGTTCTCTAAACAGAGATTTTATTTTG
TTATCATGGTTGTGTTTATTTGGGCTAAAACAACTTTAGGCTTCACAAGAGCTTTAGAAAAGTCAAACTT
TTTAAAATCCTTAAAGAAATAATTATATTTCTTGCTCATATAGGTGATTTTTTCACTATAAGTATTCTGT
TGTATTCCACTTGATATATCAAGATATGAAAATCATTAATAGTGTACAACTCATCTTCTATCACAGCACA
ACTAACACGTTTTTATCATAAGAATATGCAAGAAATAATAAATTGAAAATGTTATACAACTTAATTTTTC
TATTTGAAGTTTGATAGTCTTCATTTAAATAAAAGTATTTTAAATTCATAAAGAAAGAAAAATATAATTA
CATGTTACTAATACCATCATCTTACTATTACTATCATGCCATCATAACTTTACTATCACTTCAACTACTA
GCATAATCATATAGCTAGTATTACAACCATCATAATTAATATTATTATTTTTATAATTATATCCAACATT
ACTACTACTATTATTTCTAGTACTACCTAGTAATTTCAATAGAGATTTTTTTTAAAATATTTTTCTTAAT
CCTATAAAGAGATCATTGAAATATTAATTTATATTTCATTATATCATTATTGTTTCTTTCTTTATATATA
TTTGATATTGTGATAGCTTTTACAATTACGGTTGAAATTAGTAGATTTAAAAAGAAAAACACTTTTAGTT
GTAGTTTTTTAAAAAATAGGTTTTTTTTTAAAATATATTTAGTTAAAATTTCTACATGCAAAATAAATGA
AAGAAATGTCATTTTAGTTTTTATAAAATTAAAATTTGAAATACAATATGTATAGGGTCAATATAAAAAA
TATAAAATATTTACCGAACTAAATAATTTTTTCTTTATAGATAAATAAATAATAAAAACTAGCACAATAC
TGATCGATATCTTCGTGGTGATGGCATTCTTATACTCATCAACCATTCCCAAATCCCAATAGTACGAGGA
AAAAGCCACACGCGGGGTGGGTCGATAATGGCCGTTCTGTAAATCATACAAGAGCAGAAGGAAATGACAA
CGCTGGCCATTAAAAGCGGCAGATGAGAACATGGCTGTAATTCATCTGGGAAGATAGCAATGGCTCAGAA
AGATCATGCCATGCCAAACAAACACAGGCCATTCATCCCTTTCCCCTCCACTCTATCCAAAATTCTTTCC
CCGTAATGAATATTCTATTGCAAGTGCCAATGCGACCTCGTTTGAAGAATGCAAGAGCTAGCTAGTTTGG
CCCACTTACAAGAAATCAAAGGTGGAGGCCCAAAGATTGAAAACCAGATGCTGTAGAGAGTCTCAAGACT
CTACAGTCTACACACGATTATGCAATTTTACATGATTGGAAAATGGAATGGTAGAGATTTGGAGACACAA
ACGCAAAGCATAACCACAGCCATACATTGAATGAGAGGCTATCCATAATCTAATCCTCTCTTATTTTTTC
TCCAGTCTGTCTTCTCTCAAACCACCCTTCTCTACTTGTAGTGCTGCTACACCAACACAAACACTACTAC
TACTACTACTAGTAGTTCGAAGAGAATTTCTGCTTTTGTCAGTGTGTTTATCCTACCATA
Sequence ID: 48
Sequence Length: 3168
Sequence Type: DNA
Organism: Poplar sp.
TGATTCGCTTGCCTTTAGCTTTATTTCTTAATCATGGAAGTCATATATATAATCAATCACGAACCTTTAG
CAGCGAGGAGGGGGAGGGAAAAAAAAGATTTCAGAGAACGAGGACCGTACACGTGGAATTATTGACCTAA
AATATGAAGCTAAAAGACCAAGGAACCGATGCAGTCCAAGTTGGTTTTCCAGATAACTGCTGCGACTGTC
GACGAAGTGAATAAATTAATAAAAAGATTTGGAGACCAAGATGATTCCATGTATTTATAGGGCATTGAAC
ATGGCTCTGCTGCTTGTAGCTTTGAATCACATCAATTGTTTCTAGGTCTAACCAGAGCTGGTGAAATGCG
CAATTGGCCACCATGAAAATTCGACATGTCTCTAGTCTCCTCTTCTTTCTTCAAAATTCGGTGGAGTTCT
AAATCCTCACCTCGCCTGTTGCATGAATTTAGATTTTGAACCCTCCAAAAAAATAAAAATCAGCCGATGG
GAAAATGTCAACGGTGATTTCTATCATGGCTGCCATTCCATCAAGAATATAATCATTGAGAGATTCATCG
ATGAAATTATCACGCCAAACAAAAAAATTCTATTGACAATAATCTCATTAGTAAATTCACTGGATCTTCA
CCCGAACCAATGAGAGTTGCCAACAATTACTCGATGAACTTGTTCTCGTATGAACGCTTAGACGATCGGT
GATTGCTGGCTCGATCCAGATTGTGGGGTCCTAACGATTGAGACACTACAACACACTCGTATATCCTCGA
TCGTAATGTTGGAGATACTGCAAATCCAATTTTATCGGGTCCATCGGTAAATCTAGCCTCCAACTACAAA
TCTGGATCGAGTACTAGATGGATCAAATATGCATCCCCGTATCTCTTATGTAATCGAGTGTTATATATTT
CTTGGAAAAAAGTCATCAAATATAAAAAAACATAACTGAAAAAATATGTTTAAATCATACGTACCATGAA
CTTCTTGACTCGGGTATTTATGAGCTACTGCACCTCTTTCTGTTTGCTTTTATTCTGCTTGTGAGTTTCA
ACAAAAAGCTCCATTGGTCTTGGATCGCGCCCAAGCACTGTAGCCTGTAAAAGAAAGATATTGTTGGTTA
AATATTTTCATATAATACAATAATTTTAAAAAGAATTGTCATATAAAACAAAATCTTACCATTTGTTTTG
CATGTGAAATGAATATAATGAATCCGCTAGTGTGTGTGGTCATGGAAACATAAATCTTCCTAATTCGGTT
CTTTGAACTAAATGATGATCGCCTTAAGAAATGCTCTGACATCACGCGTTATAGATAAACATCCTAGATA
CCTTCCATGACATGTAGAGTCTTGTTTATTTAAAAACATTATCATCGATGAATATTTTTTGTATTAAAAA
AATTGACCCGGCTCATACATACCACGTGACACTCTAGTATTACCTAAACTCCATGAATGACATCAAGAAA
ACCATGGTATAGAGAGATGACATTAAGAGAGAGAAAAGGTAAGATTAGAGGAGGAAGTTAACATTTAACA
CTCTCCCTAATTTCTACCTTTTATTATAGAATATTTTGATTGTTATTTTTTTTTCTTTTTTGTCTTGAGT
CCATCTAGAGTCACAAAGGATTTTGAACTATACAGTGAAAGTAGCCTTTTTCTTATTATTATATAATTGA
GAGGATTAACGTAAGATAGAACTTTCATTTGCTTCCCTGGAAAAACTTATAGATACCTAAACATTTGACC
AAGCTTGTGTGCGTGCATCTTCTCTATTAAGTTTCAAGATTTCAATCATTCTCCACTTACGTTAGGGTGA
AAATAAGACTTGTATTCTCTCCGAAACCTGTTTGGAAATATAGTTGCGGTTACTTTTTAAAATGCTTTTT
ACTTAAAAAAATATCAATTTTTTTTTAATTTTTAAAAAATTATTTTTGATATCAGCGTATCAAAATGATC
TAAAAATACCAAAAAAAATATTAATTTGAAGCAAAGAAAAAAATTAAAAAATTTTAATTTTTTTCAAAAG
CGCTTTTGAAATACAAAAACAAACAAATAAAACTGCTTCTTCTATTATTATTATTATTATTATTTTATTT
TATTTATATATAAATTCAATATTTGATCCATGGCTTCTCTATCATTATTGCCATCATCGTTTTAATCATT
TTACTTCTATCATGTAATATAGAATACAACTTGTTTTTTTATAAAATATTTTTCCCAACACAAACAAATA
ATAAAAAAAATAAATTCAATAATAAGAAACACATTGTTTACGAAACAAACAAACTGCTAAATATTAATTT
ATGTTTCTTCTATGCATCTATATAGCCTATAATATTAAAAATACGAAAAGTTTTTAGAGCTTTGAGGCTT
GTTTTCATATCTATGAAGTATTTGAGAGTGTGATAGTGATTATAGTTTAAAGTGATTTTTATTTGAAAAT
GTATTAAAATAATGTTTTTTTTTTTAAAATTATTTTTAACATCAACACATGAAAACAATATAAAAATATA
TAAAAAATAATTTTAAATAAATTTTTTTTAAATTTTTAAAAAATATAGTTTTAACCTCATTCTCTAACAT
TTAAAAACACCATTTCTTTCTTAGTGGTGGTGGCATCTGGGTACTACCGTTTAGTTAAAGCATCATCCAT
TTGAATCCATCCAGTTCACTCATCAAGAGCCAATACGGCCTCTATATAAAACTCTTTCCCCATAATGAAC
ATTCTATTGCAAGCACCAATACGGCCTCGTTTTGGTGGCTTAAAATCAAGTGAGAAAAGAGTTGCAGGCT
GTTTTATGACAAGATTGAAGGATGCAAGAACTAGTTTGGCCCACTTACAAGAAATGAAAGGTTGAGGCCC
AAAGATTGGAAACCAGTTGGAGCCAGATGCTGCAGAGGCTCTCTACTACGCACAGTACTCATGCAAGTTT
TGGAAAATGGAATGGTAGAGATTTGGAGACACCAACACCAAGCAGAACCACAGCCATACATTCAATGAGA
GGCTATCCATAATCTAATCCTCTCTTATTTTTTTCTCGCCTGTCATCTCCCAAGCCACCATCCTCTACTT
CTAGTGCTGCTAAACCAACACTAGAACTACTGCTATTTGAAAGAAAATTCCTGCTTTTGTCAGCCTGTTT
TGTTTCCATAATACCATT
Sequence ID: 49
Sequence Length: 3000
Sequence Type: DNA
Organism: Poplar sp.
GAGTCAAAATCAAACCATCACCTGCTCCTGCACTTCAATTTCTTCTTCATCATCTTCTTCTTTTGGAGCT
TCAAGAGAAACATCAACAACAATGGCTGCTGCTACTGCTATTCTTTGGAATCCTTTAATTCTAACCAGAA
ATCCTCAAGGACTCCACCTATCTCCAGGTTTGTAGTCTTCATTTTTCGCTTACTTCAATTCAATTTTCTG
TATAATTAGGTTTATTCGAGGTGGCTAGATATTCCCATGGCTTCCTTTGTTTGGCACACAGCATTTGTAA
ATGAAATATTGATTATATTATTTATTTATCTCTGTTAGCGTTGTCTTCTTCTTCTTCTATAAAGCCGTTG
GACAAACGCGCCTTGAGTGGTGGTCCTGGAAAACTGCGAATAACTAACTCCATAACCAGAAGCTCACTCA
CAGTCCAAGCTGCTGCTAATGCTGCTTATAGGTACCGTACAATTGCTTTGATGGAAATAGAAGAATTACT
ATAAATTTAGCTGAGAATTGCTTGTTTGCCTGTGTTTTCACAGTGATGGAGGAAGAAGGCCAAGCAATGC
AAGCATTTTTGTTGGTGGCTTTGTTTTGGGAGGACTTATAGTTGGTGCTCTTGGATGTGTATATGCTCCT
CAGGTCTTGTTAATTTTTTCCATATTTTTCTTATCTCTTTCAACTAAGCGGTGTGTGCTTCTTATAGCAT
AGTTGAAGAGAATTTTACAAGTCATTTTGCAAGTGAATCTTGTTTCTTATGCTTCTGTATAGATCAGCAA
GGCATTAGCTGGCACTGACCGGAAGGATCTTATGAGGAAGCTGCCCAAATTCATCTATGATGAAGAAAAA
GCTCTGGAGGTGTGCTTTAGTTCTTGATTCTCTCTGTTCGTAATCAATTTTTGGCATTTTAACACTTTAG
ATTCAACCTTGCCTTGTGATTTTTCGGTTCTTTTATGTCGCTTTTTGCTATCCCCGTTTCTTTATTCATT
AATGATCTTTGGGATCCGGACTCACTTCCTATTTACCTTTCAAGTCGTTGCTACTCTATGAAAGTATTTG
TTGATCGTTGTCCCGGAAAATTTACTTTTCAGCTCATTGCTGCCATGTCATAGCATTTGCTGATCATTGT
CCCAAAATAGTTATGAAGATTAGATTCCTTAGGAGTTTGATTATTTTTGCTTTTGTCTCTGGTTTGACAT
ACAATGCGTAATTGATTCCTTGGCACCATCACATTGAGTGCGTTGCGTGCCTGACATTTTATGATCAGCA
TAATGACAGACAGTGTTTGTAATTTCTTGATTTCCAGAAAACTCGAAAAATATTAGCTGAGAAGATTGAA
CAACTGAACTCGGCCATTGATGATGTTTCCTCTCAACTCCGATCAGAAGATGCCCCAAATGGAGCAAGTG
TGCATTCTGATGACATTGAAGCTGCCATATGAGCTACCTCTGTCTACGCGACAGTCTACCTGGTCTCCAC
TACCAGTAATTTCCATACTGATAATTAATAGCAGTTGAGCTGATCTTTCTTTGTTCTCTTCTTTTCAATG
AACAGTTCCACTGTGACGGCGCCTGGTGTGGGTACATGTAATTTTGCGTGATCGGTTCTGTTAGATTACG
CTGATCAAGTATTGTACTCGAGTTATATACAGTAACATTCCAGCTTTTTTATTTCGTTATATGAGGAGCT
GTTTTTTTCACTACAGTTGTGCTGCTATAACTAGAACTCTCCAATGTTATGTGCCCAGGGGTCACACTGG
ATTCTATAGCCTATCCTTGAGCCAAATTCCTCATGTCCTGGGAATTTTTTTTTTCAAAAATGCTTGATAG
TTAACATGTTCTTCGGGGAGGTTATATTCAGTACTTTAGAAAAATTTGGGCACTCTTTGTATGATACGAA
ACCTGCGTGATTTGATTTCAGACTGTGAGTCAGTCATGTTGCTAAATTTGTGGAGTGATCAAGCAACCAA
CATATGGATTGCTTGACAGCATATGAAGCAGGGAAAATAATCTGCACTATGCGGTGCCTTCTTCAGTCCC
CTCCTCCTGTCAAGGACTGGCGTTGCCATTTTTGCTAGGCAGGTCTCCCATAATACTAAATGATATGAGC
TGTTTACTCTCCGAAGACCTGGGCTCTATGCTACTAATGATTGCTACATCAAGTCAAATGATTTGTTTCC
TTTCATATTGTTTTTCTGAAAATATTGCATAGACTAATCCCTGCCTCAGAAAACTCCTAAATAGGAACCT
CTATTTGAGACGTCCATGCAGTTGTTCTTCCTTTCTGACCTTAATCCACATAGATATTTTATATTTCCCA
GTGTTCTCCACCATGCAGAGTGACTTTCCCTGTTAACCAGAAAAATTCCCAGGAAAGATCTACTGGATGG
GAGCCTGAATGGGGGCCATTTCTACTCATCATGTGGCCCACGAGGTACACAGCATTTCTCACTCAGCAAA
TTAGTGGCTATCAAGCAGTTGTATTAAACCCAGTCTGGGTCAATTCAGGATCTATTTGACTCGAAACCTT
TCTAAACCAGTTTTGAAACCAGATTATATGACGTGTTTAATATTTTTAAACCTTGATTAATCGGTCTTTA
ATTATTATTTTTTAAAACCAAGTCATTTCAACTTTTTATTTTAAATATCGAAAGGATATAGTTTTAGCTA
ACTTATAACCCAAGTTTTGGACTGCATGAATCACATGACGGGTTTAATACCTTTGCAATTAAAGGGTCTG
ATTTTACAACTGTGAAGAGAGCAGCCAAGGAAATACAAAAGGGTGCACAATCTGTGGTCGAGACTCTCAC
ATTACAAGCTCAATTATCATCTTATCCCATACATCCTCATGCACCATTCTCACCACTCGTTCTTTCAACT
AAGTTGCGGCCATAATTTTTTTAAAAAAAATTGGAAAAAATATCAAGGAATTGTAAAATGAGAATAACTG
ATAAAACGAGTTGAACTATATATAGCCAAGAAGTGGCTCTCTTTAGCTTTAGTTGCAGCC
Sequence ID: 50
Sequence Length: 3053
Sequence Type: DNA
Organism: Poplar sp.
GTACTCTATTGAAGAAGTCTCCAATTCCCAAGCCTTCCATTTCTTCATCTGTTCAACAACCCCAGTCAAC
AAATTCTTCAGAGACGTCTACCATGAATGAATTCTAGATGCATTGCTAGAATTTTATGAACTATAGCTTA
CACACATAATATTATTAAATTCAGTTTAATTCAGCATATTAGTCGATTAGAAAATCTTACCTGAATTAAA
TTTTAAATTAAACAGGTAAGAGTTGACTTAATCAAATATGGTTAATTAAATAAATGAATTTTTGATCCAA
TCAAAATTTAGTATGATTATATTTTAAAAAAAGATATTATTTTAATTATTTAAATAAATAATAGTACGAA
GCCTAGCTGAGTAGCATTGTGTTGTGTATGTATATATAAATGCATAGAGAGAGCTAATTATGGAATTGAA
GCTTACTTTTGCTTGCCCCAGTGCCATAGTAACGACACTGTAAAGAACATGAAAAATGGCAAGAACAAAT
AGAAATATGTGCAATTGGTGCACTCCTGTTTGCGAAATCAATGAAACTTTACCCTGCAACCAAATAAAGA
GAAATAAGGGATTAATTTATCATATTTCCACCTTCTCCATATTCGTTAATTCTTATCGATTACAAGAAGT
TAGCTTTGTTAATTAATCACCTTGGAGCAATAGCCGCCCGCCGCCAGCTGCCGCTAAAACTCTGTGGATT
GCTACATTTCCGGCAAATGAAAATAGTTTCCTCTTATGATCACCTCCATCATTGCCGTCACCTCCTTTAC
CACCATTTTTATCTTTATAGTTGTCCCCAACGTAATCATATTTGCAGGGAAGCATGACGTTTTCGTATTT
CTCAGGGATGCATATCTTTAAGATGGCGCTTGTACCCACAGTAATTAGCAACGATATGAAGCCCAGTAGC
ATTAATTCTGTAGTGACAAACATACTCGTCAAATTAAACTAAAGAACCCAGCAAGGTGTAATTTTTAATA
ATTATAGACGCAGCTAAGCACTCGTCACCAAAAATGTTGTTTCTAGCAGCGATTAAAAAGACTTGCCAGC
TTTAATTTTCTCCAAAGCTTCACTCATGGCCTTCTTTTGGCGTTTTTGAAACCACTGTAGTGTGCATCAA
AGCACAGGGGAAGTGAGAACAAATTAAAATATAGAGCTGAGCACAGATTTTAAGCCGATGTAAGAATCAC
TCACCTTTCCTAACGACTGAATCCCATGTTCTATGAGAACAGATAAGACCACAAACACTGCACAAACTGC
CGCTACAGCCCATGTAGGTGTGTTTTGCAAGGTCTTGTCTCCATAAGAACCAGCTGCCATGGTTGCTAGC
CAAATTGCACTCCGTTCAAACTTCAAGCACTTGGTAGGAAAATGGGCAAACTAGAAATATTTCTCGGCCA
AATTTATGGAACTGATGACCAATCATGGAAATGGAAGGCGCGCAATGGTCGTAGATTTATGCTTAAGATT
TGCCAACAACAACGCGGTTTTAATGTGTAATGAAATTTGTCATTACCTTTTGTTGTTATTACATGAGGCT
GGGAATATTAGCTTTGTCATCTGAAATTCCGTCACTTGGGACCCCTTGACATGAGCCGGCTGAGTCAAAA
ATCATTAATGGGATGGATCGAACCATTGTCATGGCTCTACATTAATGAAATAACCTTCAAAGTTTTGCAT
ATAAAAAAATTACTATAAAAATTGGCTTCTTGCATACAGTATTTTTCATCAATCTCTGGAGACTTGATGC
ATCATATGTGGTTAGAGAATGCGTTGAAAGAGTCAAGAAACTAGGCAGAGGTTACCATTTCAAACAAACC
CACTAAACTAATATCTGTCGTTTTGTGATTTTAATTGTTCAAGATTCGATATAGTTTATCGAATTTATGC
TTTCCTGTACTACGCAATATTGCTAGTGATGTTCTTCCTTGATGTAGTTGCTTGAAATAGGATTCATGCC
GTGTGTCGGAGGATGACATGTGAAGAGAAAGAGTATAGTTGTTAGACATGATTCGTAGATTAGAAGTCTT
GAATTAACGGGATCATCAGGTGAATTTGGGTTAATCATGTATAATATCAAAATAATGTCGGTTTTTTTTT
TCTTAGTGTTAGTCAATCAGGTCACGAAAAACTTTACCGGTCTAAATTTCTTATTCGGTGCAACTAGAAA
CCTATTTCGAGTTAAGCTATAGTTTCTGAATTTTCAAGTTTACCAACTATAAGAAAGAGATATTTGAGCA
ATCTGCGCCCCTGCTGTGCCGTCCGCACATGCTGGCATTGCCATTTGTTTTGATAGTTCTCCCACAATAC
AAAATGATGTGTTCTGTTTACTTCCAGGAGGTCAGGGCTCTATGCCACTAATTAACGAGTGCTTCATATA
GTCACAAATGATTTGGGAGGTGGAAGTGGCCGACGGATTGTTTCTTCTTGTTGTAGTTTTCCTTTTCTTG
TATTTTTTTTTCCTGAAAATTTTGCATGGACTAATTCCTTCCTCGGAAAACTACCAAATGCGATTCTTTA
TTCGAGACATTGATGCACATGTTCTTCCTTCCTGACCTGAATATTTGACATGGATATTTTGTATTTTCCA
GTGATCTCCACCATGTACAATGGCTTTCCCTGTTAGCCAGAAAAATAAAGAGGAGAAATCTACTGACTGT
GGGCCGTTTCTCCACTTCATGTGGGCCACGAGGTACAAAAACTACGTATCTCACTCAACAAATTAGTGGC
AGTTAAGGGGCCGATGATTTTAGAGCCGTGAAGAGGGCGTTCGGGAAAATACAAAAGGGTGCACAATCTG
TGGTTCAGATTCTCATATTACAAGCTCAAACATCATCTTATCCCATACATTCCCATGCACAATTCTTACC
ACCACACGTTCTTTTAACTTAGTTGCAACCATACTTTAAAAGATTTGGAAAATTATGAAGGAAATGTAAA
ATGAGAGTGACTAATGAAACGAATTGAACTGTATTTATAGTCAGGAAGGAATTGAACTGTATCCCAGCCA
ATACCAGACTTCCATCCAAGACCAACCACTCTTTCCCTACTCA
Sequence ID: 51
Sequence Length: 3042
Sequence Type: DNA
Organism: Poplar sp.
CTTTATGCATACCAATATTGAATTAAAGGGGCGAGAGTGAATTAAAGTCGCTCAACATCCACTTTCAAAT
AATTGCTGATATTTACCTAATTATACCAGTTCAAAGAAAAAAATATATACAATACTGAGATATAGAATTT
CAATTAATCAAATAGGAAATGATTTAAGTATTTTAACACAATTTCAAAAATATTTTTTAAAAACTATTGC
TCCTTCCCTCTTTGTGTTTATATTTATCTATTGTGCTTTGTTATTATACTCTATTGTGTTTTTTTAAAAA
ATATTTTATGTTTTAATCAGAGTTTTAAAACCCGGACCGGCTCGGGCGGGTCGACCCGGGACCCGGCCGA
CCCGGGCCTGGGATCGGTCCTGGTGGAGGCAAAAACCAGCTTGGGAATTGGTCCGGAGAAACCCAGTCGA
CCCGGGACCCGGTCCACTTGGTCAACCCGGGTGAGACCGGTCAAATTTTTTTTTATATTGATAGTCATTA
AACGACGTCGTTTTGACATTTTAAAAGGCCAAAACGCTGAAGAGTGAAGAAAAACAAAGCAATATCAGTT
ACAATTTATAAACCTAATTAACTCAACTATTTGAAACCTAGCTGAGGAGCAGATGAGCAAAGGACGTCTT
GACTATTGATCGCTGTTGTTTCTCTGCGAAAAAGGTTTGAATCTCCTTCTTTTACTATTTGTTTTTTTTT
ATTCTTGGCCGTTGAAACAATTTGTTTTCAAATATGGAATTAAGCACAATAATAGCAAAACACCATCAGT
TAGTCGCCCCCTTCCCCTCTCAGTCTATCGAACAGCTGAACTGCGTGCCTTGTCTCTTTCTGATTTAACA
ATGGAGGAGACCGACGCTTCTGCTCTCGGCGAGAAAACCATAAGCAAAAACTTAGTCTCTCTCTCATTTG
TTCTTACTGGAAATTTTGTTTTCTATTTTTTTTTAGTGATGGATTTAATTGTGACTTTTGGGTATGTTGC
TACTCTTGTAGTGCACTAAAGAAGGAATAAAGAATAAGAAGAAAGAAGAAGAGAGACGCCAAAAAGAGGT
GTTTTTTTATAGGTTTTTTTTTAGTTGACCCGGATCAACCCATCTGACCCGTGACCAGATCACTTGACCG
GATCAATAACTGGATTGGGTTTCAAAACTATGGTTTTAATATTTTAAAACAGGAAAAGAAACAAACTGGC
CCAGACTGGCTGACGTGGCTTTCGAGGACCTGGCCAGGGTTTCTCAGCTCCGAAAGTACACGACAAACTC
GCCTCCGTGTTTCACACAGTTGCAATCCCCTAATGATAGTTGTTCTAGCACTGTCTCCGTACAAACAATT
TCACGGAATTTGTGTGGAGCTCTGTTTTTTCTAGTTTTACATGTCTATTGTAAAGAATACCAAGGGCAAT
AAGCAACCATGACTGATAATAAATTTCTAGTTGGATAAATATGCTCTTCGAATATCTTATTTTTTTTTAT
TAAAAAAATGAATAAATAAAATAAACACTCGCCATAGTCATACTTATGCTCTTTAATTTTTTAGTATATA
TCTAAATTATTTTTTACTTGAAAATATATTAAAATAATAATTTTTTATTTTTTACAATTTAATTTTGACA
TCAATACATAAAAATAATAAAATAATTATTTTAAAAAATATCAAAAATACATTTTAAATAAGTTTCACTT
AAAAACCTTTGACCCACTTTTTTTGCCATTTAACCAACCGAATATAAAATCAGGAGAACAAGAGAGCTCA
CACCAGAAACAATGTGCCTGCTCGTACATTAACACAACTCACCCGCTGATAGTTTGCTTTTTTGGAGAGA
GAGAGAGAGATACACAAAAAATAAAAAAGTGAGCTAAGGACGTCCCTCATAAGAAACTCCGCTAAAAATT
TCCTTCTCTTTCTGTTAAATACACTTTCGATTTTGCTTTTAAAATACGATCAAACGCCACCAACAACTAC
CATTTCTTTTTTTTGGTTCTTTCATTTTCCTCGCCTTCTGCTCCCTCTCAAAAGCCGCAAACCAAGCCTT
GGTTTAGTAGTAGGGTTCTTGAGAGAAAGAGCTTGGTGTTGCACTCAGTACAAATGGCTTTCTCTTGTCT
TCTCAGATCTACGCCCGCCGCTCCTCTTGTTGAAGCTTCCCGCTCCGACTTCTCTCCTTCACCGTCCGAT
CGATTCAAGGTATCTGCTCCTTTTTCTCCTATCAACTCTTTCTTTTTGAAAGTCCGTTAGTCGCACTTTG
TTTGAGCTTGATTTTGGTTTTTGGTTGTTGAGAGAGAAAAAAAAGTAGAGTTAAAACTAGATATTGAAAT
TGTTATTCATGAAAAAATAAAATAAATATGGAAGTTGCCTGACTTGGAAGTTAAATCTGCAAATAACGAG
AGATATGAAAGATATAAGATAGATAGTAGTGTATACAGGTTATTTATTTATTTATTTGATTTCAGTTATA
CTGGTTTTGAGTTACTTATATCTGTTTGGTTGGCGAGAAAACTGAGGAAAATGAAACAGAAGCTTGTTTT
AAAACAAACTGTTCAAAAAAAAAAAAGGTGATATCAGATCGGTATTATCATCAATTATTATTAAACCCTG
TGCTTCTTTAGCTTATAGCTCTTATATTTTGAGTTTATTTAGTTTTAATAATTTCTAATCGTTATAAAGA
ATGCATTAATGTTCATATATAGACTAAGCTTTTGACAATGTGTCTGTCTATAGCTGATGAGTGGTGTGAA
GTGCCAGTTACTATTTAGAGATGTATGTAACTTTTGGCCATTTTTAGTTTTAATCTTTCCTTTTCTCCTT
GTAATCTCAGGTGTCTAGCGTTAGTTTTAACTCTTTGAAGAGCATTTTTGGCACTTCAATTCCAACAGGG
TCATCTTCCTCACAGTTGAGTACTGAAGAATTTGATATTAAAATCTCCACATATATATATAGGTACATGT
GCGCCAGCATGCCTTTTCTTTAAACTAATTCTGAAACATTTTGAACTACTCAGGACATGCAGTGGAAGGA
GCATCCAACCCATCAAGGCCACAGCTACCGAA
Sequence ID: 52
Sequence Length: 3099
Sequence Type: DNA
Organism: Poplar sp.
CTGTTAAGAGGAATGAAGAAGCGTGTGGCTGCAACAAATGAATTGGTCCCTCTGATGGCTCAAAGCCAAA
GACTCTCTTTGAAAGATGCCTCCACATTCTTCATCAAAATTGATTCAGAGGAGCAGCAAGGAAAATAAAT
GGCTTGAGCTTCGCCAAATCATAATTTTTTCACTGAGACTTATCAAACTTAATCAATCCCCTTGCTAGGG
ACGTTGGTTTCGGAACACTTTATTCTCGTAATCAACGACCATAATTTGTCTGGCCTTTTGAATATTTTAA
CTTTTGTTTCGGCCAAGGGCTTGGCCTCTGAATATTCATTTATTTTGGATCATGAACCTAACTTCTATAA
TTTTACCAGCATTTCTTCTGAACAATTGTCATGTTTTAGTAGACTTTGGTGAGCAATTCACCTTGAATCA
ACTGTAAGTTCTGATGGAATAACAATCTCGACACAAATGGGCTAAACTTAGCACAAGTTATTCGTGAAGT
TCTACATATCCAGTGCTTCTCCCGGATGTTCATGTCAAAGCCCTCGGGCGATTCTCGATCATCCTCTTGA
AAATATTTATAATTATTTTATTACCGGCCGGCCTCTGCTTGGTCGGTCGCTTGTGCATAGTAAGGTATTG
AAGTGTTCCATTCTACTATGCGTATGTTCGCAACAAATTCTTTTAACTCTTTTGTCCGCGAAAACTAACC
CTTAACGAGAGAGTGGTTTGTGGTATTCCAAATCCATCGACAATATTTTTCTTAATAAAATTTATTTGCT
TTAACCAATTTAGTAAATAATCACATAATAAAAGTATATCGTTTTCAGGAAGGACGATAATTTGTTTCAA
TCGATGACCCAACTTTTAAAAGATTATATTTTATTTACAGTATGATTAGAAGTGTTGTTATAATTATTTT
TTAAAATATTTTTTATTCAGAAATGTATTAAAATAATATTTTTTTATTTTTAAAAATTATTTTTTATATC
AGTACATCAAAATAATATAAAAATACAAAAAAATATTAATTTGAAAAAAAATATATTTTATTTTATTTTT
TAAAAAATATTTTACAAATTTAAAAACAAACAGAATAGTACCATAAGTTATTTACTCCTCGGTTATTATT
TTTTTTCTTAATTGCCGAGTATGCCCCCGAATATGTAAAATAGTTTTTTTCTTTTCTTTTTGAAATGCTA
TCCATGGTATTGATTGAAATAAAGATACTCCTCAAATACACCGACGCCCGACACGGCATCAGATATTAAT
ATAACTCAATTTTTGTTTTTCAAGTAATTAAATAATATATTCATTGATTGTACATTAAAGAAATCGAAGC
AATTGCTAATAGAACTATAGGGAAGAAAACACAGCAAGAGTGGAGGAGCAAAGCCGCCCCGGTGGGCCCA
GAAGAAAAATACAAAAAGGCCCCAGACTGGCTGACGTGGCCTTCGAGGACTTGGCCAGGGTTTCTCAGCT
CCGAAACTACATGACAGACTCACCTACGTGATTCACCGTATTTTTTTTTATTAAAAAGATGTTCATATAA
ATTATGGATATTTCAACTAATTTCATGAATTTAAAAATTAAAAATTATATAATTTTTTAGTTGCTATTAT
ATTAACAATCACGTGATCCAAACCTGAAATCACAATGAAAATAAACCTCTTGATCCCAAACTGGTCCGGA
CACCTATATTATTTTGAATATTTTTGCAATTATTCAAGGAAATATTTTCAAATCATTAATTGTTTCAACA
TTAACAAGAATATATATATTTTCTTATGGTTTTTTACTTTTTTAAATATAGATTTATTCAACGCCTTATA
TATTGAATGAGTAAAGAAATTAGACATAATAATTGTACCTTTACCAATGTTTATTGTATGTACATTAAGA
TAAAATAAGAAGTTATTTTTTTTAATCTAATTTGCTCACTAAACGCACCATGACGACTCTACAAAATTAA
TTTTAAAATACAACTTGGCCATCAAGTCAAATATATTTTGAAAATAAAAATGTAATATAAATAAATTTCA
CTAAAAAACCCACATATTATAAGGGAAAAAAAAAATACAAAGAGAGCTCACGCCAGAAACAATATGTCTA
CTCCCATGTTATCATAACTCACACGCGGTTTATTGCATGGAGAGAGAGAGAAATTCAGAGAAAAATACGG
TATTTTAGAATTAATAACACATGTTAGAAAAACATTAAAAAATATTATAATTTAAAAAAATATTATAGTT
TAAAATATTTAATGATCAAGAAATGAGATTTAATATTCAATAATAATAATAAAAAAGTTATCAAGGATAC
ATCGAAGCTCTGTTCTTTATTTATTCAGTGTTGTTTGAAAGATCTTGACAAGATAAATCTAACCACATCT
TAAAAGATGATCAAATATAGTCATAATTTGTTAAAAATTTTATTCGAGGTTAATTCAAGATTAATTATAA
TATTCTCTGGTGTTCTTCCAATATATAACTAGAATTGTTTCGTCGAGATATTTTTAAAAGTAACGAGTGT
GTTAAAACCAAAGTTTGTAGTAACATATTTTTTTTTCTTCTTCCATTTATTTCTCCTACAATATCACATC
ATTTCATTTTTTTCTTGATTATTGGATTTTTAATCTTATATTTTGTTTATTGAAACTTCTTTGATTGACT
TTTGCTAAATAATATGTAAAAGAAAAATATTTTGAAATATCCACTAAATATATTTTGAGATGCAATGTTA
TTTCAAATAGTAATTCTCTATAAAAATAATATTTTAAAATATAGTTGTTTTTAAATTCTCCTATTTTTTA
AGTATTTTTTATTTTTTTTCAAAACTTTTAATATACTATGCTATTTAAAAAGAAAAGATTAAAAAAATCG
GTACAGGAAAAAGCGAGCCAAGCACATTCCTCGTGAGAAACTCCGCTTAAAGTTTCCGCTTGTTTTATTA
AAAAAAAATACACATTCGTTTTGTTTTTAAAACACCATCAAACACCACCTGCCTGCAACTACCATTTCTT
TTTCGATTTTTCATTTTCCTCTCCCTCTCAAAAGCCGCAAACCCAACCTTTGCCTTATCAGTAGGGTTTT
TGTGTTGCTCTTAGTACAG
Sequence ID: 53
Sequence Length: 3035
Sequence Type: DNA
Organism: Poplar sp.
AGATCCTCAGAATCCAACTCCTGTTACAATTCAGGCATTTCCTACACATTACTTGCTCATGGTTTGAGTC
GTTGTCATTCTTTGGTTTGAGTCGTTGTCATTCTTGGCTCCTCATCTGTCTAGTCAATTGTTTGGGGTAC
TTTAGCATATAGAGACCTTAGAGAGGCATCATTTGCTCCATGGTTTTGTGGGGACAAAAAACAAAGAACA
CTGCTTGCACTCAGGATTTAGGTTGCCTATTGACAAAGGGTTCTTCCTAAGTAAATTCTTGATTTCTCGT
AAATGTCTTTTTCTTCGTAGGGATGACTTGCAACGCTTACTCAAGTAGATTTGTAATTTTCGCTACCTCT
CTTCTTAAATTGTCAATTTTTCTCTGAGGATTCATTTCTAACTGGGCCCTTTTTTCGTGTTTCACTTGCT
TTTCTCTGAGGATTCATTTCTAACTGGGCCCTTTTTTCGTGTTTCATTTGCTTCTCTCTGAGTCTTGTAT
TGTAGAGTCTGAGAGGGAATTGGATTCAGGAGGGACATTTATTCTACATTCTACTACTGGAGCAATTTGC
AAAGAAAATAAGCAAGAGATGCAAGAATGCATGATGATGGGTGCTAAAATGATTGTATTGGATATGGATT
TTGTCTGATTCTGCCATGGTATACGGAACCGAGATCTGGTAAACTGCCTTGTAGATCCGTATAAAATTGA
ACCTTTAACGTGTATTAATCCATCCATTTTGAGAACAAAATTAAAGATTTTCATTCAATTAAAATGTGGG
AAATAGCTTAACACATTATCCATACATTTTATGAAGTTGGTTTAAGACTCCCTTCATCCGAAATATGGAA
TATCATTTCAAGCCTTAACCCATATCACTTGAGAAGAAAACTTTAAAATTCTATTCATTTGAAAATTAGG
TAATTTACATATTCTCCAACATCAAACCATTAAAATTTAATCCATCCATTTTGAGAATAAAAATTCAAAA
TTTCCATTCAATAAAAAATGTGGGAAATGTGTTGACGTATTATCCTTGCATTATGGGCAAGTAGTTTAGA
AATTTCATTCATCTGAAAATATAAAAATAGCTTAATGCATTAACTCGTAGATATCAAAAATAAAATTTCA
AGATTTTCATTCAACCAAAAATAAGAAAATATATGCAAAATGATCTATTAATTCATCATATAAATTAACT
CTCTGTTTCATGAGTATGAGTGTAAATTGTGGAATGTTCTTAAATAGATCTTGATTATCTTGTTATCTAT
CTCATGAAATTTCATACACGTCGAGCTTGTCCAATAATATTATAAGGAGACACCTTAGAGGAAGGTTCTA
ACCATTACATGTAGATAGGTTAGGTACTAACCATGACCTTGTAGACTTTCAATAAAAATCGAGGTTTCAT
GAGTTCTAGTCCACAATGAAACCCAGTAGGACACCGGTAATTTTTTTATTGCTAGCTTAAGGCAATATGC
GTGCAAAAAAAATAGATGTAGTAGGAGCAAGGGCATAAATTCATATCTAGACGAAGCAAACATGAAATGC
ATTCAAAACAAAATAATTGTATGAAAAAATCAAAAATCAAATTAAACAATTTAGAAAAAAAAACAAGTAA
ATATTCAGCATATAAAGCACAAAACATACATTATAATGGAATCAAACAAACAAAGTTTATTCCTTAAAGT
TTCTAGTAGAGTTATCATAATGGCATAGAGGACATCTTAGTAAAGTAAAGTAGAGGTAAAATAGAAAATA
AGGATATCATAGTGTCACGCAGAGTTTTAAAACCCGGACCGGTCCGGTGGGTCGACCCGGGACCCGGCCG
ACCCGGGCATGGGACCGGTCCGGGTGGAGGCAAAAACCCGCTTGGGAGTTGGCCCGGTGAAACCCGGTCG
ACCCGGAGGGTCGACCCGGGACCCGGGCCACCCGGTCAAACCCGCCTAAGACCCGGTCTCTTTTTTTTAT
ACTTAATAGACGTTAATCGACGTCGTTTTGGGCTTGATTTTGCGCCACTGCTATTTTCGATGATAAAGCT
GCAAATTTGACACAAAAACAAGAGAATCACAAAAGGGTTGTTCTTAGTTTCTTGAAAAAGTGGATAAACA
AGTGGGTTTCTGTCAATTACCTTGTGATATTGTAAAAAGCGAAGTGTACAACAAGAAGAAGATAGTGGAG
AGAGGAAGCTTAGAGAGTGCCATAACTGAGGAAGAACTTACGTTTCACCACTTCATCTCCTGGTTCTGGT
TCAGCTAAAGATTCTTGCATTATCTTGTAGGGTCTTGTGGTTCTCTTTTAAGTGAATCTTTTAGGTAGGT
TCTGGTTGTTTTTGGAGTTTGCTTGCTGGCCTCAAGAACGAAGAAAGAATTTCAAAGAACTCCACTTCTG
ACAAGAGTGAATGATATTGCTGGCCAAATCTTAATAATAGCCCCCCAACTATGTAGCTAATAACAATTTA
ACAAAAGGGGGAAGGACTGGAACAAGAGGAGGAGCCTTAGGCCCTTATGGTAACTTCACCTAGGGTGACG
TGGCGAGTTCTGGCTGGCATTTTCAGTGAGAGAGACGTTAGAGAACTAGCCCACCAGACAGCACCATCTG
TCTTTTTTTTATAGGTTTTTTTTGGGTTGACCCGGGTTAACCCGGGTCAACCCATCTGACCCGTGACCCG
ATCACTTGACCGAATCAATAACCGAATCGGGTTTCAAAACTATGGTGTCACGGAGTCTGTCATTATGTCA
AATATTCTCTTCCTTCCAAGCTTACTAATTTTTGCATTTTCAAAAAAAAAATCTTGTTTTTGGAGTCCTT
AACCAATTCTAAACCGCTGGTTATCGAAATCAACGGTGCATGATCCCTCCAATTTTAGGAAATTACGTGG
TCATCACGTAATCACCGCCTCGACAAGTCCGCTCAACCTAACCGTTTCTTCTTAAGATCGCAAGAATCGC
TCCTTAATTCCGTTGTATAATATTTATATATAAAGAAGAGCAAGCACCTCACTCACCATCACACTACACT
TCATTTCTTCATCATCATCATCATA
Sequence ID: 54
Sequence Length: 3090
Sequence Type: DNA
Organism: Poplar sp.
TTTCCTCTTCTTTTTTTTCATTTTTTCATTTTTTTCCAACTTTTCCTTTATTTTTTTGTTTATTTTTTTT
TTTCAAAATTACTTTTCTTTTTTTTCCAACATTCCTCTTTTTCTTTTTTTTTATTTATTTTTTTTCAAAA
TTATCTTTGTTGATTTTACTTTTTAAATGTTGAGCTGGTTAAAAATTTTGCTTTGTAATTTTTTTCCTTT
AAAATACTATAGATTGTTACGGTGTTTTCGCACATAGTTTTTCTATTTTATTTTTTTATTTTTTAAAATT
ATATTTGTCGATTATTTTTTATATTGAGCTGACTGAAAATTTAGTTTTGTAATTTTTTCCTTTAAAACAT
TGTTGATTGTTACAATGTTTCTCTGCGTGGTTTTTTTTATTTTTGTTTTTTTATGATTTTCTTCGAAATT
ATCTTTTTTTATTTTATTTTTTAATATTGAGCTGGTTAAAAATTACAGTTACAATATGTGAGGAAAGCAC
TGTAACTTTCCTCGCAAATTACTGTGGATTGCTACAGTGTTTTTTCTCATATGGTTTTTTTTTCTGTTTT
GTTATGTTTTTTTCTAAAATTATCTTTGTCAATTTTATTTTTTAAATATTAAGCTGGTTAAGAATTGCAA
TTACAAGTAAATACAAGTTTTCCTCACAAAACACTATGGATTGATACAGTTTTTCCTCACATGATTTTTT
TCCAGTTTCTTTTGTGTTTTCTTTTTTTGTAATATTTTTTTCTAAAATTTTCTTCGTCAATTTTATTTTT
TTTAATATTGAGTTGGTTAGAATTTAACTTTGTAATAAAACTTAATTATGTAGGGAAAGCACTATAGTTT
TCCTCGCAAAACATTGTTGAGAATTACAATTATAAGTCATTACAAACAAGGCTAAATCATGTGGGAAAGC
ACTGTAGCTTTCATTACAAAACACTGTGAATTGCTAGAGTGTTTTCAACATGATTTTCCTTTTTTTTGCT
ATTTTTTTTTAAATTGCCTCTGTTGATTTGATTTTTTCAATATTGGGCTGATTAAAAATTTAGCTTTGTA
ATTTTTTTTTCTTTAAAACACTGTGAATTGTTGTAGTGTTTTCCCATATGATTTTTTATGATTTTTTTCA
AAATTATCTTTGTCGATTTTTGTTTAATATTGAGTTGGTTAAGAATTATAATTATAATGAAGCTAAATCA
TATGGGAAAAGCGTTGCAGATTTTCTCACAAAACACTATGAATTGCTACAGTATTTCTCTAAATGGTTTT
TTATTTTATTTTATCGGGGAAAGCATTGTAGTTTTCCTCACAAAACATTATCAATTGCTACAGTGTTTTT
TTTCATAGGTATTTTCATTCCAAAATTATCTTTGTTGATTTTTTTTTAATATTAAGTTGGTAAAGAATTT
AGCTTTATAATTCTTTTTGCTTTTTATTAACAGAAAATCTAAATCATGTGGTGAAAACACTGTGTCTTTC
CTCCCAAAACACTGTGAATTGCTACAGATCATTTTGTTAAGTCTCTAAGTTTTTTATCACCAATATAACT
TTTTTTCCATCATAAAATATTAATTCTATCATACTTTTAATTTTTATTATTTATCTAATACTGATACATA
ATTATAACATTATTAATACTTGTGTTTATAAATTCATGGCAACATGCGCGCATGCCATCTCATGATAATA
AAATACAGGAGTTCTTAAGGTACATTGGTTTTGCCACTTGCATGACGTTGGGTTTCTGCTCAGAACGCTG
GCTGACCTGTTGCCAAGAATCTTTGTGAGCATGCCATGGCCCAAATTGAATATAACCCGAGTTATATATA
TATATATATATATATATATGGGTTTGACCAATAAACAATTTCTTCGCAAATGTTTAAAAGAATTATAATT
ATCTCCTTTTAGGATCTCTGTTATGCTAGTTTGAAAATGTAATTGTGATTATTTTTTAAAAAAATTTTAT
TTAAAAATATATTAAAATAATATTTTTTTTTATTTTTTTAAAATTATTTTTGACATCAGCACATCAAAAT
GATTTGAAAACATTAAAAAAATATTAATTTGAAATAAAAAAATAAAAAAATTTTATTTTTTTTTAAAATG
CTTTTAAAATACAAAAACAAATCAAATCTAAACACTAATACTTTCATTAAATTGGTATAAGTCACGGCAA
CTTTCACTCAATAGATCTTTCATGTTTTTATATATAAAAAAATTTAGACACTAATACTAACTTGATTTTA
TAATTAAGTTATTTCTTATATTAATTTCAACTTAAAACTGTTTTAATTCGTCGTCAATGACTTCTGTTAA
AATCATCCAACAAGAATTATTATTATTATTATTATTATTTAAAATAATGTTGTTTCTGTTTTAAAAAGTA
TAATTTTGGTAAAAAAAATATAAAAGTTTTAAAAAATTCACATAAAAGTTCTTAATTATACTCTTTTAAT
TTAAAAGTGAAATTAAAAAAAAATCATTTGACAATAAAATCGAAAGGTGGGTATTTTTGAGATCTACTTT
TCAAGTTTCTAGCAACCGGAAAACCGTGAAACTTGTGTGGCAGCAAATTCAGGCTTGACCTCGCAGGCTG
CTGATGTCAGCAGAGAGCTCATGCAACCAGTCCACGACAAGAACCTTCCGTTGCTGGGCCCAACAACTTT
GAGCCCATCGGACATACTCCGAATATTCCCACTTCCAGAACATTAAGACTCCCACGGTCATTAAGGAAAG
TGCGCTATACCTTCCTCTCACGCCTCTCCCTCATAGATAAACAACCAGTCAACCACCCAAGACTGAATCC
AAATGCCACAAAATCGCGTGAATTTCCTTTTTAAAATCAGCGGCTAATACAAGGGCTTTTCAAAATTCCA
ATCAGCGGCTTAAAACCAAGCTAATTGTTTCTTTCGTTTTGCCCAAGAAAAACAAAACCGCTCACGTGAC
CTAACCCGTCACCACGCACCTACTCTTCTTTATATGACCCTCAATTAGCAGCCACTCTCCTCACTAAAAT
TATCTCTGTTTAAGCTTATCTCTCAAATCTCCCCTCTTAATTCTAAATCTCTCCTCTGTGTCCCGTTACT
ATCAACAACA
Sequence ID: 55
Sequence Length: 3079
Sequence Type: DNA
Organism: Poplar sp.
GAGTTCCAAGAAACAGAGGTCTGTTGCACGGTCTTCCACCGAAGCTGAGTATAGGGCCATTGCTACAACA
GCTGCAGAACTTGCATGGATTCAATCTTTCCTGACAGAACTCCAATTTCCTCTCTCATGCATACCGGTTA
TATATAGTGATAACATTGGTGCTAATTATGTTTGTGGCAACCCAGTGTTTCACTCTCGCATGAAGCATAT
TGCTATTGATTTTCACTTTGTTTGTGACAAAGTGGCTCACGGTTCTCTACGTGTCTCCCATGTCGCTACT
GCTGATAAGCTTGCCGATGCTCTAACAAAGTCTCTGTCCAGACAACGTCTCTCCTTTCTACTGTCCAAGA
TTGGTATCTCCAATGGGAGCTCCATCTTGCGGGGGCGTGTAAAGGATATACACCATCCACTCAATACAAG
TTGACTAGTTCAGCTTGTAAACCGGCAACAATAATTCAAAAGAGTTAATTATCCTTTATCCTTAGCTGAT
GTAACAAACCTTGTAATCTACAGACATGTATTAATTTATAAAAATAACCTAAATTTATTTATAAAGTGTT
TTTTTTAAAAAAAAAATTATATATTTAGATTATTTTTATATATTAATATCAAAAATAAATTTTAAAAAAT
AAAAAATATATTATTTTAATATATTTTCAAGCGAAAAACACTTTGCAAAACACGCTATCACACTATCAAA
CATTATCTTAATTTATAAAAATAACCTAACTTTATTTATAAAAAGAATTATATAATTATAAACTTTTGGC
GATAATTCTGTGAGCATAAAATTTCTTTTGATAAATTAATAAGTTAATAAAAATCACGTCATAAATCACG
GGGAAAAAAGTCATGGAAAGATTTTATGAGTCCAGGTTTGCTAGCTTCCTCTTCAAAATGCGCACGCACA
CACCGTCGATCATACTGCCTCACCGGGCCATGGATCGACCCCAAGCCCGCTTAGGTCCATGACCTTATGG
GCCTTCACCGTACATTGATGTAGAGTGGAAAGAAATCAGGCGGTCCACATATCATCCACCTAGATTCTCG
TGCCCGTCAAAAATTGACGTGGCCCATGATATAGTCCACAAATAGTGACGTGGCCACCAATCACCCCTCA
GCTTTTTCTTAATGGACATAAACAAAAGTCGTTCCCTTGGAAACATGACACCACTCCTGTACCACTTGAC
TTTTTGTTATCGACAACAGCCGCTTTGAAGATCACGTTATCTTCACTCAGATCAACGGCTAAAATTAACC
TCAACGTCTCACTATACCTAACAATCAAATCTAACCGTGTATAAAAACAAGATCAGCATGACCCATTAAA
GACATGGATCTCATGAGAGTTTGACCAAACTCATCCTCCCATTTTTAAATTTGGTCAAATAGATCCACGT
ACTTCATTTGTTTTTTCAATTTGAACCCTTAACGTTAAATTTAAATTCAAATCAATTCTTCTAGCTAAAT
AGTTACTAAAACAATTAAAAGACATGAGATTGATAATAGTGAAAAAGGTTGATATTATTTTAATTGCTTA
GTTAATATATTCATGTCGTAGCTATACTTTTTGCTCAAGATAAGGTCGTGGATTCGATCAATATTAAGAG
ACTAATCTTTTTTATTTATAGATGATTAGGTTAATTCGTTAGTTTAATTTTTTAGTTAACAAATCCGTGT
AATAGCATAATGGTTACCACATGTGATTTAAGGTGAAGTTAAATGTTTGGTCCATGTTATTAGTAAAAAA
AAAAAATTTAATCATATCATTTAATATAATTTATTTATTTATTTTTATCATTATATATTTTTTATCTTTT
ACTCCCCATCATTCTAATATGAGGTTTTTAACTGTTTTTTTTATTTTATATTTTAATATAAGATTTTTAT
TTAATTTTATCTTGTAGTGTTAAGTTGATTTTTTCTTTGAACTTGCTCAATAAATAAGGTTATGATGAAA
AAACTCCCATATAATTTAATTTAAAATTCATATTAATTAAATAGTCAGATTTGAATTTTTCAAGTTTAAA
CTATTATACCAATATCACAACAACACCAAAAAAATTCAATAATATAGATATTATTTTTTTATTTAATTTT
TTTTAATCTAAACCACAACGAGAACATGGTAAATTTAAAAATAGCTTTAATTTGAGGACACAAATTTGTT
AATGAAGGAACCTGTATAGAATGGAATAGATAACATTCTAATACAATGTTTTGCAACTCTTTTATTTCGA
TAAAAAAAAATGGCTAGAGTTTACATATCATATGTACAGTAGCAAAAATTATAACTTATAGTTTACATTA
ATTCATTCTAAGTGATTTATGCTTTAGAATACAATAACACATTGACACATACACTTATATAAAATAAACA
CATAGAAACTTAATTATCAACAAAACTATATTAAGTATGAATAAATCAGACTTAATACCATCAGATTTTG
TTAGAGATAGAAAATATATATCTGAAGCATAGTTTTTAAATCCCAACTTAAGGTCATGGTCACAAGTCTT
GACCGGATCATTCTAAGTCAATTTCCAATATTTTTTATAAATAAAATGATATTATTTTTATTTTAAAAAA
TAAATAATCAATAAGTTGTAATTAAATTTTTGATCAGGCTGGGTTACCTGGTCATCCAGCTTTTTTACCA
AGTCATTCAAGTTTTATTTTTTTTTATTTTTTTTTAAATTCAGCTTTTCAGCCTGATTTTGACCCTGATG
AACTCAAGCTGTGTTCTAAAACTACCATATAAAGTAATATAAAATCTCTTTCAATTTATTCCTGTGCACT
TGGAATGTAAAAGCCTAAAAGGTGGCCAACTCTCCTTGGAGGAACTAATTTAACAAAAAGAAAACATATA
TATTGAGTTTAATAAATCCCAAAACTCAAGGGATCACAACTGAATTTAGTCAACAGTCACCGGCTACTTC
GGGACCTCGGGTACCCGGTTGGCTCTCTTTTTATACTTGCAAGAGAGCCTCGCTCCTATCGCCCACTACC
TCACTTGTATCTCCTCACTGACTCCAACTCTCATACTGTTTCTCAGCGTGTCTCGGCTTCTCTAGAATC
Sequence ID: 56
Sequence Length: 3085
Sequence Type: DNA
Organism: Poplar sp.
AAAAATTATGTAAGTTTCTTAACTTTCCATTCAAATTGCAAACACAAATATAAATATCACGCCCCGTTTG
TTTGCAGGAAAGTTGTTTCCTTTTAGAAAGTGAATTCCGGAAAAGTGAATTTCAGGAAAGTGAATTATTT
TTCGATGTTTGGTAGTATAATTGAAAATAAATTGGAAAACATTTTCCAGTGTTTGGTTATGTTATGAAAA
ATGAGCTGGAAAATAACTTATTAATGTTTTATTTTTTCAAGTTTATTAAAATAATGAGGAACAAATCTTA
CAAATTAAAAAGTTGAATGAGGATGAAATTGAAAAAAAATATAATTTCATAAATTATCTTAAATAAAATA
AATAATAATCAAAATAATAGAGATCAAATCTAAAAAATAAAAAAAATGAAAGATGAAGAAATTAAAATAA
TAATAATTAGCATTTCATAAATTATTTTTTTAAAAAAAGTAACAATCAAAAGAATGAGGACCAAATTTGA
TAGATAAAAAATTTCAATAAAAAAATGATAAGGAAAAAGCAAATAACAATTATAAAAATAAAGACCAAAG
TTAATATAAAAATTAAATTTTAAGAGATGAAATTGAAAAATAAATATTCAAAACAAAATATATATAGCAA
TCAAAAGTCCGAGGACCAAATTTAATATAATCAGCAAATAATATGACATTTCTAAATTTTTCACAACTTC
CGGAAAGTGTTTTCCGTCCAAATTTTTCAGGAAAATACTTTCCTGGAAACCAAGCCAAATTTTCCTTTGA
CTGAAAAGTGTTTTCCGTTGATCAATTTTTCTAATGGCAAACAAACACAAGAAAGTTTGGAAAGTGGTTT
CCCGGAAACCACTTTCTGGAAAACAAACATAGCCTTAATTGGATATATTTCTCTGGGATTTATGCTCAGG
TTTTTTTTTCACTGTTTTTTAATTAAAAAATAATGAAATTTATTTCCTGATGACAAAAAATAACAGGCCG
TGGATCTTTTCTGATTGGGAGTGTGGTTGTGGTTGTTTTTCAAAGTGCTTTTCATTTAGAAATGTATCAA
AATAATATTTTTTATTTTTTTAAAATATTTTTAATATCAGTATGTTAAAATGATTTGAAAATATCAAAAA
TTATTAATTCAAAAAAAATAAAAAAATAATTTTTTTTTTAAAATATTTTTGAAATACAAAATCAAGTGAT
TAAACATAGATACTTGTGCCATCAAAAGTGACGTGGCCACCAATCAGCCCTCAGCTTTTTCTTCACGGAT
ATAAACAACCCTAGGAAATACCACCACTACCATCCTACTTTACTTTTCACAATCGACAACAGACCACTCT
ATAAATATCACGCTATCTTCACACAAATCAAACGGCCACAAATATGCCGCAGCTTCGCACTCTACCCCAC
CATCAAATCAAACCGTCTATTAAACAAGATCAGCATGACACATTAAATAAGACCGGCATCTCACAAAAGT
CGGACGACAATCGTCCTTACATTTTAAGTTTGGTCAAATAAATCCTTTTACTTCATTTAGTTTTCAAATT
GAATCCAATTCCTCTAGATAAACTCGTGTTCATGAAAAATTGTTGCCATGTGATCACACATTAAGTAATA
CTCTAAAAGGCGTGGGGAAACTACTGTAGCTTCCCCACGCCTTTCACTTATTAATATATTATATTATTAT
AATTATTATATTATATTATATTACAGAACTGTTTTTTTTTTGTTTTTAATTTTTTTTTGTTTTTTTTTAA
TGAATTTTTTTTGTTTGATTTAGTTTGTTAATATGATAAAATTTTTTTAATGAAAATCTAATAACTAATT
TTTGAAGATTTTCATGATACGGATCCCGGGTTTGATGGGTTAACCTGATTTGACGAGTTAATCCAGATTT
TTTTTTTATTTTTAATTAATTTTTTTATTTAGTTTAGTTTGTTAATGTTAAATTTCTTTCTATTTAATTA
TCAGACTTTCATGACACGTATCCTGGGCTTGACGAGTTAACTTGGTTTGATGGGTTAACCCGGTTAATTA
TGGGTAAACCCGTCAATTTTTTTTTCTATTTAGTTATCAAACTTTCATGACGCGAATCCCAGGTTTTACG
GGTTAACCCAATTAATTCAGATTTTTTTTTCTTTAGTTTTTTTCTTCCTGTTGGTTTTTTTTTTTTAATT
AATCTATTTAATTATCATACTTTTATGACACGACCTTATAGCCAGACCCACATCCAATATTCTTGGGTCC
GGTGTTGCAGCCAGACTTACTTAAACTTGGGTCATGCAAGTTTAATGTTATTATTAATATTATAAATATT
ATTTTTAGGTCAGGCGTTGCAGCCAAACCCAAGATTTTTGGGTATAGCTTTGCAGAAAAACCTAACACTT
TTAAATTTTAATTTCTTTTTATATTTTTTATGCAAAAAAAAATCCGTCGGGAGGGTCATGTAACTAGTGT
AATTATATAAATGGATGAAAGACTCCATGGGATCCGTTTAGTTTGGGAAAGTTGTGGCTGTGGCTTTTGA
TTATATTCCAAAGCTACAAATTATAAAAATATCTTTAAAATCATAAGCGTGATATTATTTTTTAAGTATT
ATTAAATGCTTGTTTCGCAGCTAAAAATATCTTAAAAATCATAAGCGTGATATTATTTTTTAAGTATTAT
TAAATGCTTATTTTATAATATAATAAACATTATATTTTAAAGTATTTTTTGTATAAAAATATATTATTTT
ATTTTTTAAAATTTATTTTTAACATCAACAATCAAAATTATAAAAAAATATTTTAAAATTTTTAATTTTA
AATAAAAAAATTAAGCAAATAAATCTTTTTTTTCGGAGTTAAAAAATAATTTTATAAAAATTTAAGTTTT
TATATTTTAAATTAATTTTTTTATATTATTTTGATGTATATCAAAAATAAATTGTTTTTTAAAAAATATT
TTAATATATTTTAAAAAACACAGAGAAGACGCTGTACTTCTGGTTGGCTTCCTTTAAATATCCACAGCAC
TAAATTTTCACTTCTATCCTCACTAATCTCTCCACTGCTTCCTGCTTTTACTCCTACTGTTTATCTCTGT
GAATC
Sequence ID: 57
Sequence Length: 3049
Sequence Type: DNA
Organism: Poplar sp.
AATATCAAAAAATAATTTTTTGATAAACACAATTTTAAATAAACGGAGCAAAAAAAAACACTGTGCTGAC
TATTCTTCTTGTTCTGAAGAATTGTTTATGTGTCAAAAGAGGCCTTAAAAAGTTTAGTTTTTAAAAATTT
AGGGTTTGTTGAGTTGAGCTTTGCATAACTCAAATAGAATATAACCTGAGTTCAAGATATAATCCTAAGG
TGGATCATCAACTACCAATAAACAATTTATCGCTTTTTTAATCAATCAATTCGCAAATGTTTAAAAGAAT
TTAAAGTCTCCGTTTAGCAGAATAATGAAATAATTCTTTTTATTGGATAATTTTCAACTTTCTCTTGAAA
AATTGGTAGCTTATCGTCTAATTTCTAAAATAAAATTTATGAGGATTCATTTTGGAAAAAAATATATAAA
AAGAGGGGAGGGGGGATGTTTGATTTGAACTTATTGCAATGTTTTAATAACGACGACAAAGACAAGAAAT
ATGCCTCGTGTGTGTGAGCTCAGACACCGCGCGTGTGTTTGTGATTTTTTTATTTATAGTGGAGTGTGTT
TCTTAAACTATTGGTCTCGTGAATCAATATTTGTCTGATAATGAAAAATAAATCTTGAGCCTACGTTACT
CATTATAAGTTTCCAATACATTATTATATCTCATTTATAGTGGGTGGGGCTTGTGTCGATATTCCATATG
CTTAGACATGATAGAATATCAAGTTTGGATGATATAAGTCTAGTTTATTTTTAGAACCAATACTTCTAAA
CATGGCTATGAGTAAAGCCCATGTGAGAATAGGTCTGATGAGTTATCAGACCTAGTACCCCTAAGCCTGG
GTGCGTGCCAAGCCCAAGCAAACAAAGATTTGACAAGCTGTCAGACTCATCATTCTTGTACATTGGCATT
GTATCGAGCCCAAGTGGGTGTGAGTCCTCACAAGTTGTCACACCTAGCATTTTTGTGTGTGATTGCACGC
TGAGTCTAATTGGACATGTGTTTAACGATTTGTTAGACCATCTCTTTTAGGCTTGGCTGTGCGCCAAGCC
CAATCGGGTAAGGTTCCGACGACCTTCTCGACCCGTCAACTTAAGGAATGGTTGTGTGCCAAGCCTAGTT
GGACATGGGTACGACGTGCTATCAGACCTAACATCCTTGAATTCGGTCAAGTGCCAAGTTTAACTGAGCA
AAGGTTTGACTCGCTGTCATACCCATTGCCCGAGGGCTTAACTTTGTGCCAAGCACGAAGGGATAAAAAG
ACCTGGAAATCATTATAGACTCCATGTTAGGCCACCAAGCTCAATTTACTTATTCTTATGATTTTTTTAA
TATAAAATGCTAATATCATGGGTATTTGCCTCTCATCCCTCGTTAATATTAATGTTATGAGTATCTCATT
CCCTATTAATATTAGTGTTAGGGATATCTCAACTCATATTAATGTTAATGTTATAGATATTTATCACACT
AAGTGTTGCAATGGTAGAAAGACCTCTTAAACTCTTAAGAAATATCTTTTGTACATGGTATTAAGAGTAT
AAATACTCCAAAATCACACAGGCAAAGAGTTTATGATCTTTATTCTTAATAAAAAATACATTCTTAATTT
TATAGCATTTACTGCATCAAAACTAAGAATAAACACTTTTGTTCTTAAGAATAAATGCTTATTTTTTTTA
TTTATTATAATAATTTGCTTAAAGGTTAATAATTTTATCATGTGAGGATTCCAAACTCATAGGGATTGCT
TTGTAAATTATATGTTCTTTACATAAAGCTACAAGTTCCTAAGCAATGAAAAATAAATTATTTTGCTAAA
ACTTGTGATTAACACACAATACAACCCCTAAAAAACTTATTCATGATTTTGGAATATTATCCATTGTAAT
TTTTACTTGTTAAATATTTTATATTTTTTATAGGAGTTAATTATTTTATATTTAATTGTGTATTAAATGA
TAAAAATAATTCAATATTTACATGAACTTGATATTTATTATTTTATTTCTTATTTATCATTAAATACTGT
ATCATGTTTATTTATATGAAATACTCAAACCCGCTTTATTGACATTTCTAACACGTGTTCATAGATAAAG
TAATTTTTCTTTGGATGTAAAAAAAAGTCAACAAATATTTATTTCGAATCACCGAGATTGTCCTCCGAAT
CATGTTAGTAAATTCCAACTTGTTTACCCAGTCACCACGCAAGCTGAGGCTCGTGAAAGCTGATATCAGC
AACCAGCAGAAAGCTCCAGCTGTCCACGACCAGAACTATCTTTTCATCGGGGGCCGCCATATTTGAGCCG
CCAGACATCACTCCGAACAGTCCCGTCTAGAACACTAAAACTCCCACTCTCATCAAGGGATGTGCAATAC
ACCTCCCTCCTGAACCACGTCTCTCATAATTAACCAACCACTCAAAACTAACTTCAGACAGCACAAATCG
CGTGAAGTTACTTTCGTGCCGCCCACAACAAAGAGAGGAGGAGGTGTTCGCTATTGTTTTCTAAAGTGCA
GAGAAAGTATTAAAATAGTATAATTTTTAATTTTTTAAAATTTATTTTAATATCAACTTATTTAAAAAAT
TCAAAAAATTCAAAAAAATCAATTTTTAATAAAAAAATAAGTTGAAATTCAGAAGAAGAAGAAGAAGATG
AAGAAGAAACCGTTAACGTAACGTAAACGCAACTCTCTCATGTACCTTTAAAGCTCTGTTTCTAGTAAAA
TCTCTCATCTGTTTTCCGTTTCTACTAACGGCAATGGGTAAGAACAATGCCCTCTTCACCCCGCAATTTC
TCGTTTGATTTTCTCATGTTGTTTTAATTTTCATTTTCATTTTCTCTGGATTTAATTTCTTTTTTTCTTT
AACACAGCAGCATCCGACAAGAAGATTAAGATCGGTATCAACGGTATTTTTCTTCATCTTTCTGACATGT
TTTGATTTCTTTTTAACATGCATTAATTTATTAAACATTAAATAAAATAAAATGTGGATAGGATTTGGAA
GGATCGGTCGTTTGGTTGCAAGAATTGCTCTTCGAAGAG
Sequence ID: 58
Sequence Length: 3000
Sequence Type: DNA
Organism: Poplar sp.
CTAGTTTTTTAGTAGCTATTATTATATGTTCGTATTCCGCAGCGGGTCAATATCAATTTTTTTAAAATGT
AAAGAATATATTAAGAATATAGAGAATGTTTTAAGAGTCTTGAGTCTGACAGTCATGCCAGACCCATGAG
ATTTGGATCTAGCAGCCACACTAAATTCAAGCGTCTTAGGTTTGACAGCCTAGCAAGACCCAAGTGCCTT
GGATCTTGCAGCCATGCAAGACCCAAGCGCCTTGGGTCTACTTGGGTCTGATAGCCATGTCAGACCCAAT
CGCCTTGTGTAGTTTTGTTTAGGTGTGTCTAGGCTCGGGTTCAGGTCTATTTAATTTTTTTATATATATA
AATTTCATGTGGATTAGGTGTAGTGCTTACCAGAATCTGAGCAGATGATGATTAAAATGTTGAGACTTAT
AATTTTTGTATTTTTTATATATAAAAAAAACAATCAATTATCCTTTAAGTATCATAAACCGAATTCCAAG
CTAAAATATTATTTATAAACAACAAATCATCATAAAAATCTAATTTATTAGATCTAAAAATTCAAATCAT
ATCTTCCTATTGAAATTGTTCATTTTTTATGTTCATGAATTTGTTATTGCTTCAACAGAAATTATTTCTC
TTTTTTTTATGAATTTTTTTATATTTTTTTAATCAAACTCAATGCAAAAAAAATGTTTATTTAATGGGTG
GTCGTTTGTGATTTTTCACTAACCACAATATTATTTTATATTTTTTAACACCACTGGACTATATGTATTT
AGTTTCATTATACAATTTATTTAGTTTTTTAAGTGATTCTTCTGCCTACTTTATGACATCTATTGCTATA
TTAAAGAGTCCATCATTTCATCGCACGATGTTAGAAAGAAAAGATATGGAGCCCTCTTGAATGGAAGACT
TAACAAATCTAGAAGAAAAGATATGGTTGTCATATCTAGATGCGGGTCTGGCATACCTAGGCACGAATCT
AGCATGGTTGTCAAGCCCGGACGCGTACGTCCAGACCCAAGCGACTTGAATCTAGCAACCATGCTAGATC
TGAGCGCCTTGGATCTTGCAAACTATGCCAGACCCAATGTGTGTGAGTCTAGCAGTCACACCAGACCTAA
GATACTTAAGTATGGTAAGCATGTCTGATTTGACAAATAATAAACAAAGATGACAATTGTGCATTATAGT
TGTCAGGAGAGAGAAAAGAAAGAAAAAACGCAAACAATCGATCACCAAAGGCAATCTAACCATTATCTGT
CTACATGTGCCACACCATGTGGAAGAGAGCTTGAATTTGCATCTAATGAGACAGCGGATGGCTAGATTTG
GCCACCGAGAAGCATCACACGCACCTCTTTAATGGCGTGGGTGCCACCCACTCGCTGGGGAAGAAAAAGA
AAGCCAAAGATATGTCTTGTGTGAAAAGATGAGAAATTAAAAGTGAAACTACTATTACAATTCACGGTTA
AATGTCTCTTGATCTATAGTGATTTTCCCACACAATTTAGCTTTTGTTATAATATCACGGGACAAATAAT
TTATCTTATAGCAAAAAAAAAAAAAAACATGCAAGCTTTTCTAACATGTTTTTTTATACAAAAAAAATAA
TCATAATTATAAATAAAAAAAGCCTACGCATGCATTTAATTAAATAAATCGAGAATTATTTATGCTAATA
AATGCAAAAAAAATGTTAAGGTATCTATTAGACTCACTTTACTGCAAGTCGAATATTTTTTTATAACACA
AAAGAATAAATGTGTAAGTGTATTTTCTGACATCGAGTAAAAATTATAACTTTGAATTAAAAAGTTGATG
CCTACGTATAAAAAATAAAGTGAAGATCATATGCGTTGCTAAAGACGTGTAACATCTCAAGCAAGTTTTT
ACTTAGTAAAAAAATGAGATAATGCTGCAATTTGTATGGTGAAATATCATTTTCTTAGTATTTGTATAAT
AAGTTTTTTTTAAAAAAAAGTGTAAAGGGAGAAAAAGAAAAAAATACAAAAGAATGAAGATAAGAATAAA
AAGAATTACATAAATATATCAAGTGTAGAGTGATAAATATTTCAAGTGTAAAGAATACAAAAAATATTTT
TTCTTCCAAAAAAAGAAGGAAAAACTAAAAAATAAATATTATAAAAGAATGAAGATAAGAACAAAAAAAT
GGTGATAAATAGGCAAAGTGTAAAGTGAAAAATATGTCAAATATAAAAAAGAAAAAAGAAAAAGGGGAAA
AATCTAGAAATTATTTTTTCTAAAATAACAGATTATAGATTTGTCATTGCTTTAGTAACAACATTGAAAA
AAAATTATAGTTATGTCATTTCTACAGTAAAAGATTTTAACGAGGTAGAAAAATTAGCCTAACAGATTTA
GTTTGGTTGGGTTTAGTTTAGTTTAGTTTTTGTTTTTTTTTTTGGTTTCTCAAATCTAAAACAGAAAAAT
GAGCCAAACAGATTTTCTTTAAATTTTCTATGAAGTATTTTCAATTTAATATCTCATATAGGTTTTTTCA
ATTAATTTTATATCATATTTTCGACCATATCGATTATTTTGTCCACTCCCTCTCAACAAAGGTCGAAGTT
CCGTGCTGCACAACCCTAGCCATTCCCATCTCTTCATACGGTCACTTCAATATTAGAGTACAAGAAAAAT
GAGATCATGGAAGCTTGAACTCAAGATACCTTTCCCACAGTACCAATCAATTACTCCAAAAACAACTCCA
AACGTCTTCTAAGCCACGTAGAATTAAGGCTAGTCACATCCTCCAGCATCACCTAGAAAGAAGCTGGTTA
CAGAACTGTGCCTATAAAACCCCATTGCGCTGTTTTGAATTTGTAGAGCCAATAAAAGAACACTCACTTA
CAAGGCTGTAGACGCATATACTCTTATACGTACCATGGCCGCAAAAAAATGCAGTATTCATATATTTCTG
CCATTTCTCCTTATTTTAGCCACAAAAATAGTCTGTCGTCCTCTTGATGAGCAGGAATAT
Sequence ID: 59
Sequence Length: 3151
Sequence Type: DNA
Organism: Poplar sp.
GAAAGTTATTTTGTTTTTAAATTAGTGCATGCTCTTCTTTTGAAAGAAAAAAAATATTTTGACATATAAT
TAGATAAAAAAATTTGTCAGAATTCCCTACAACAACATGAGATTAAATATCTTAATCTATATTTATCTCT
TAACTTTTTAACTTAGTCTTAGATTAAAATTAACAGATTTTTAGTATTTATTGACATATAATTCTCGATC
AATTTTGTTAAATATATGCACCAAGTTTTATATAAAAACTTTTGATATGACCTATGGTGAAATGCAGGTA
AACTGACAAGTAGAAACTAAAATTAATAGAACTTAATTAACAAAAACTACAACTCTTTGGAAAAAAAAAA
CATTGTTCACCTAGATTCAAGGGCCATGTTCGTTGGAACAATGTAATAACGATTTTGCTCTTTTCATAAA
AAATAATTGAACTAGCTATTGGCGGTTGTGAGGTCTTTGCATAAGGTCCATTAGTTATTATCAAATTGTT
CGGGGCGCAAGTAGATCGTTTTCCATTTTAAAAGCACAGTGAAACAACTAAAATATTCAAGGAAGCAAAA
CTTTTGGAGGTGGCGTCTAAGGGCATTTTCATCTTTTCACACTAGTATTTTGGTGATGGTTATGTTGGAT
AAGGGGCAATTTGGTATTTTAATACATGTAAAATATAATTAAAAAAAACGTAACCGGCACATGCAAGGCA
CTCATCAACGCGTCCTGGAGGCAAAAAAAATCCGGTTTTGTCCTTTTGTATTTAATTAATTTGGAATTAA
AGTACATTGTGTTTTTCTTTTTGAAAAAGGTTTTTTGTTGCCCGATTATCACAATTACTTTTTTTTTGGT
CTATTTAATATCATTGTTTTTTTTTAAAAAAAAATTTAATTAAAATAAAATCAACTTATTTGATTTAGTT
GATTCTACGAGCTCAACATTATTTATTTTTTATACGAGAAGAAATGGCCCTGCGGTGTAAAGTGGGTTGT
TGAGCTAGTGTTTTAGCTAAAGCATCAATTTAAGCAACCTTGAAGTTTCGCAATAAAGTTTTCTTCCTTG
GAATGATTGTGAAATCAATGGAAATTAAGTTATGATCCAATCAATATCACAAATAAAAATTATTGCATGC
ATATTTCAATTATTTGGATGAAGCTGTGCCGAGGCTGTTGTAAATGGAATCTTAATTTCATCGACAAAAA
TAAAGGAATCCAAACTTATTGATTAGTACAGTGCTTGAAACGAAATCAAAAGCCTTTGCCGTGATTTGCT
CTACCAGTGTTGTCTTCGGCATTGGTCGTTAAAAAAATCATCCCACGTATCGGTTGGTTCGTCTCGGTTT
TAATTAAAAAATTAAATAAATTAAGGAGATGGAATCTTCCCCCATTCCATTAATTATTGACAGTCATGGT
TCGATCGTTGATCACACGTACACGGTACACTGGCAAGAAGAAGAAGATGATGGAAGTTCTAACCAATGGG
ATGTGTTGGTCTGTTCCTTTATCAAAATCTAAGCCCCAAATTTCCACTGAGCTTCTTGACTCCTGTGGTT
GGACCCCTAGGACCCTTCCTTGGTTGCCGTGATTTGCTCTCTTCTAGTACTTTTTTTTTTTTAGATCTCA
TCTAAATCTTGATTCATTTGTTCCTTGTCCACACAAGACGGTGATTATGCAAGTTTGATGCTTGATTTTG
TAACGAGCATGCGTGATTAATGTTTTTGACACGTTAATATACATGCAGTCATGCTTTTTTATTTATTAAC
AATTCTACTGAAATTTTAGAAAAGTTTCCTTTATATGTAGACGTACAAAGTTATTAACTACTCCTTCCAT
ATCACAAGTTATTATCACAAACATGGTCTAGTTGGTCTAATTTCTATTACCCGAGTCGCGGGTTTGGAAA
GTTAATGCTCATTGGCATTGTTTTTTTTTATATAACTAATTTTCCTTTTTGATACCATCATTTGACATTT
TTTTATATATATATAAAAAACAGGCTTTGTAATTCTCTTTGTTTTCTTTTCTTTTGGGTTATCATTATTT
CATTAACTGAGCTGCAGGTTTGACAGGTTAACTCGGGTTGACTCGGTCATGCCATAAAAATTTAATATTA
AATAAAAGCTAAAAGAGTAATAAATCAAATTTCATACACTTAAATCTTGAATTATATATATAATTTTTCT
TCTAAACATGTATGAAAGTTAGAGCACCCATCCTTTTTATCCAACTCTATATAAAATTTCATGAACAATA
GTTATAAATTTCAGAATAATAACACATCAAGATATTCGTACTCGACGTGAGAAAGGTCTTACTCTCAATT
TCAACTTAATTAGTGTATTTGGATTTACATAGTTATGAAATATTTGATAATGATTTGTTATTGACCTTTA
ATTTATCAAAAACAACTTAATAATGATAGTTGTTCACACGTATGTACACGGGCAAGAAGAAGAAGATGAT
GGAATCTTCCCCCATCTTATTTCACCAGTGATTTTCCGTCAGCAAGTTTTTTTAACATGTTATTTTATAA
AACAATTATGAAAAAATAAAGAAAAGAACAAAAAAAATGACATAAATAGACTAAGTATAGGGTGATAAAT
ATTTCCAAGTGTAAAAAATAAAAAGAAAATTTTCAAAACAAGTATAAAGAAAAAAAAGTTAGCCAACAAA
GATAATTTGTTTTTTTTTTTTACTTTTATTTTAAAAAATGCAGTTTGGTTTGGTTTAGGTTTCTCAAATC
AAAAACAGAAAAATGAGCCAAACAGATTTTCTTTAATTTTTCTATTAAGAAAAATATTGAGATCATGGAA
GTTTGAACTCCGAATCTTGATGCTACACTTATATTTTAAATTAATTGGCGCACATATTACTCCAAATTTG
ACTTCAAAGCTCCAAACGTCTTCTATGCCCCAAACGTAGAATTAAGGCTAGTCACATAGAAAGAAGCTGG
TTACAAAACTGGGTCTATAAAACCCCATTGCGCTTGTTTTGAATTTGTTGTGCCAATAAAAAAACACTCA
CTTACAAGGCTGTAGACGCATATACTCTTATACGTACCATGGCCGCAAAAAAATGCAATACTCGTATTTT
TCTGCCATTTCTCCTTATTTTAGCTGCATGGGCAACAAATAGCTTGTCGTCCTCTTGATGAGCAGGAATA
T
Sequence ID: 60
Sequence Length: 3000
Sequence Type: DNA
Organism: Poplar sp.
CGACACTTTCTTTCTTTTTCCCTCTTTTCTCTTATCTCCTCCCCTCAAAATTCATGCTAGCCATGCAAAA
TTTTGGAGTGGTCCTTTTTTTATTAAGATTTTAAATTTGACCATTTTGTAAAATTTTGATTTGTTTCAAT
TTCATCTTTCAATCCAAATTTATGATATATTTTTTTTTCATTTTAATTCTCATTCTTTTGATTTGATTTT
TCTCCTTTTGTTAAATTAATTTTTCTTTTAAATTTCACCCTTCAATAAAAAAAAACTAGTTTCCCTCTAA
TTTATTTATTTTTATTTCAATGTTGAACTTAATTCTTTTAATTGCAATTTTTTGTTTTATATCCTTTTGT
ATAATTGATTTTTTTCATAATTTCATCCCTCAACATTCGATTGGTTGGGCGTTGAAACTCATGGTTTTTC
TAGGTGGAGTGTTTCCGGTCTAATAACCTGCATTGCGGCTTTGGAAAGTTAATGCGCATTGACATTAATT
TTTTTATAACTATTTTTTTTTTTTTTTGGGTTATCATTGTCTCATGACCTGGGCTGCGAGTTTAGCAGGT
TAACCCGGGTTGACTCGGTTTTTTTTATATAGCCATTATTTTAAAGAAATTTTAATTTTTTTTCAGTCAT
GTCTTTCAAAATCCGGTTTTCTCCTTTTGTATTTAATTAGTTGGGAATTGAAGTACATTGTGTTTTCCTT
TTTGAAAAAGGTTTTTTGTTGCCCGATTATCACAATTACCTTTTTTTGGTCTATTTAATATCATTGCTTT
TTTTAAAAAAAAAAATTTAATTAAAATAAAATCAACTTATTTGATTCAGTTGATTCTACAAGCTCAACAT
TATTTATTTTTTATACGAGAAGAAATGGCCCTGCGGTGTAAAGTGGGTTGTTGAGCTAGTGTTTTAGCTA
AAGCATCAATTTAAGCAACCTTGAAGTTTCGCAATAAAGTTTTCTTCCTTGGAATGATTGTGAAATCAAT
GGAAATTAAGTTATGATCCAATCAATATCACAAATAAAAATTATTGCATGCATATTTCAATTATTTGGAT
GAAGCTGTGCCGAGGCTGTTGTAAATGGAATCTTAATTTCATCGACAAAAATAAAGGAATCCAAACTTAT
TGATTAGTACAGTGCTTGAAACGAAATCAAAAGCCTTTGCCGTGATTTGCTCTACCAGTGTTGTCTTCGG
CATTGGTCGTTAAAAAAATCATCCCACGTATCGGTTGGTTCGTCTCGGTTTTAATTAAAAAATTAAATAA
ATTAAGGAGATGGAATCTTCCCCCATTCCATTAATTATTGACAGTCATGGTTCGATCGTTGATCACACGT
CCACGGTACACTGGCAAGAAGAAGAAGATGATGGAAGTTCTAACCAATGGGATGTGTTGGTCTGTTCCTT
TATCAAAATCTAAGCCCCAAATTTCCACTGAGCTTCTTGACTCCTGTGGTTGGACCCCTAGGACCCTTCC
TTGGTTGCCGTGATTTGCTCTCTTCTAGTACTTTTTTTTTAGATCTCATCTAAATCTTGATTCATTTGTT
CCTTGTCCACACAAGACGGTGATTATGCAAGTTTGATGCTTGATTTTGTAACGAGCATGCGTGATTAATG
TTTTTGACACGTTAATATACATGCAGTCATGCTTTTTTATTTATTAACAATTCTACTGAAATTTTAGAAA
AATTTTCTTTATATGTAGACGTAAAAAGTTATTAACTACTCCTTCCATATCACAAGTTATTATTACAAAC
ATGGTCTAGTTGGTCTAGTTTCTATTATCCGAGTCGCGGGTTTGGAAAGTTAATGCTTATTGGCATTGTT
TTTTTTTTTATATAACTAATTTTCCTTTTTGATACCATCATTTGACATTTATATATATATATATATATAT
ATATATATATATATATATATATATATATATATAAAGCAGGCTTTGTAATTCTCTTTGTTTTCTTTTCTTT
TGGGTTATCATTATTTCATTAACTGAGTTGCAGGTTTGACAGGTTAACTCGGGTTGACTCGGTCATAACA
CAAAAATTTAATATTAAATAAAAGCTAAAAGAGTAATAAATCAAATTTCATACACTTAAATCTTGAATTA
TATATATATAATTTTTCTTCTAAACATGTATGAAAGTTAGAGCACCCATCTTTTTTATCCAACTCTATAT
AAAATTTCATGAACAATAGTTATAAATTTCAGAATAATAACACATCAAGATATTCGTACTCGACGTGAGG
AAGGTCTTACTCTCAATTTCAACTTAATTAGTGTATTTGGATTTACATAGTTATGAAATATTTGATAACG
ATTTGTTATTGACCTTTAATTTATCAAAAACAACTTAATAATGATAGTTGATCACACGTATGTACACGGG
CAAGAAGAAGAAGATGATGGAATCTTCCCCCATCTTATTTCACCAGTGATTTTCCGTCAGCAAGTTTTTT
TAACATGTTATTTTATAAAACAATTATGAAAAAATAAAGAAAAGAACAAAAAAAAATTGACATAAATAGA
CTAAGTATAGGGTGATAAATATTTCTAAGTGTAAAAAATAAAAAGAAAATTTTCAAAACAAGTATAAAGA
AAAAAAAGTTAGCCAGCAAAGATAATTTGTTTTTTTATTTATTAATTTTATTTTAAAAAATGCCGTTTGG
TTTGGTTTAGGTTTCTCAAATCAAAAACAGAAAAATGAGCCAAACAGATTTTCTTTAATTTTTCTATTAA
GAAAAATATTGAGATCATGGAAGTTTGAACTCCGAATCTTGATGCTACACTTATATTTTAAATTAATTGG
CGCACATATTACTCCAAATTTGACTTCAAAGCTCCAAACGTCTTCTATGCCCCAAACGTAGAATTAAGGC
TAGTCACATAGAAAGAAGCTGGTTACAAAACTGGGTCTATAAAACCCCATTGCGCTTGTTTTGAATTTGT
TGTGCCAATAAAAAAACACTCACTTACAAGGCTGTAGACGCATATACTCTTATACGTACC
Sequence ID: 61
Sequence Length: 3000
Sequence Type: DNA
Organism: Poplar sp.
ATGGTTATGTTGGATAAGGGGCAATTTGGTATTTTAATACATGTAAAATATAATTAAAAAAACGTAACCG
GAGCATGCAAGGCACTCATCAACGCGTCCTGGAGGCAAAAAAAATGCTCTTTTATTGTGTCGTTAAAATC
CTCGCAGCTTCCTCTTCCTATTAGAGCAGTGCCCGTAGTCGTTGGTGAGGTGGTTTTTTGTTGGCGACAC
TTTCTTTCTTTTTCCCTCTTTTCTCTTATCTCCTCCCCTCAAAATTCATGCTAGCCATGCAAAATTTTGG
AGTGGTCCTTTTTTTATTAAGATTTTAAATTTGACCATTTTGTAAAATTTTGATTTGTTTCAATTTCATC
TTTCAATCCAAATTTATGATATATTTTTTTTTCAATTTAATTCTCATTCTTTTGATTTGATTTTGCTCCT
TTTGTTAAATTAATTTTTCTTTTAAATTTCACCCTTCAATAAAAAAAAACTAGTTTCCCTCTAATTTATT
TATTTTTATTTCAATGTTGAACTTAATTCTTTTAATTGCAATTTTTTGTTTTATATCCTTTTGTATAATT
GATTTTTTTCATAATTCCATCCCTCAATATTCGATTGGTTGGGCGTTGAAACTCATGGTTTTTCTAGGTG
GAGTGTTTCCGGTCTAATAACCTGCATTGCGGCTTTGGAAAGTTAATGCGCATTGACATTAATTTTTTTA
TAACTAATTTTCTTTTTTTTTTGGGTTATCATTGTCTCATGACCTGGGCTGCGAGTTTAGCAGGTTAACC
CGGGTTGACTCGGTTTTTTTTATATAGCCATTATTTTAAAGAAATTTTAATTTTTTTTCAGTCATGTCTT
TCAAAATCCGGTTTTCTCCTTTTGTATTTAATTAGTTGGGAATTGAAGTACATTGTGTTTTCCTTTTTGA
AAAAGGTTTTTTGTTGCCCGATTATCACAATTACCTTTTTTTGGTCTATTTAATATCATTGCTTTTTTTT
AAAAAAAAAATTTAATTAAAATAAAATCAACTTATTTGATTCAGTTGATTCTACGAGCTCAACATTATTT
ATTTTTTATACGAGAAGAAATGGCCCTGCGGTGTAAAGTGGGTTGTTGAGCTAGTGTTTTAGCTAAAGCA
TCAATTTAAGCAACCTTGAAGTTTCGCAATAAAGTTTTCTTCCTTGGAATGATTGTGAAATCAATGGAAA
TTAAGTTATGATCCAATCAATATCACAAATAAAAATTATTGCATGCATATTTCAATTATTTGGATGAAGC
TGTGCCGAGGCTGTTGTAAATGGAATCTTAATTTCATCGACAAAAATAAAGGAATCCAAACTTATTGATT
AGTACAGTGCTTGAAACGAAATCAAAAGCCTTTGCCGTGATTTGCTCTACCAGTGTTGTCTTCGGCATTG
GTCGTTAAAAAAATCATCCCACGTATCGGTTGGTTCGTCTCGGTTTTAATTAAAAAATTAAATAAATTAA
GGAGATGGAATCTTCCCCCATTCCATTAATTATTGACAGTCATGGTTCGATCGTTGATCACACGTCCACG
GTACACTGGCAAGAAGAAGAAGATGATGGAAGTTCTAACCAATGGGATGTGTTGGTTATTATATGTAGAC
GTACAAAGTTATTAACTACTCCTTCCATATCACAAGTTATTATCACAAACATGATCTAGTTGGTCTAATT
TCTATTACCCGAGTCGCGGGTTTGGAAAGTTAATGCTCATTGGCATTGTTTTTTTTTATATAACTAATTT
TCCTTTTTGATACCATCATTTGACATTTTTTTTTATATATATAAAAAACAGGCTTTGTAATTCTCTTTGT
TTTCTTTTCTTTTGGGTTATCATTATTTCATTAACTGAGCTGCAGGTTTGACAGGTTAACTCGGGTTGAC
TCGGTCATGCCATAAAAATTTAATATTAAATAAAAGCTAAAAGAGTAATAAATCAAATTTCATACACTTA
AATCTTGAATTATATATATAATTTTTCTTCTAAACATGTATGAAAGTTAGAGTACCCATCCTTTTTATCT
AACTCTATATAAAATTTCATGAACAATAGTTATAAATTTCAGAATAATAACACATCAAGATATTCGTACT
CGACGTGAGGAAGGTCTTACTCTCAATTTCAACTTAATTAGTGTATTTGGATTTACATAGTTATGAAATA
TTTGATAAAGATTTGTTATTGACCTTTAATTTATCAAAAACAACTTAATAATGATAGTTGATCACATGTA
TGTACATGGGCAAAAAGAAGAAGATGATGGAATCTTCCCCCATCTTATTTCACCAGTGATTTTCCGTCAG
CAAGTTTTTTTAACATGTTATTTTATAAAACAATTATGAAAAAATAAAGAAAAGAACAAAAAAAAAATGA
CATAAATAGACTAAGTATAGGGTGATAAATATTTCCAAGTGTAAAAAATAAAAAGAAAATTTTCAAAACA
AGTATAAAGAAAAAAAAGTTAGCTAGCAAAGATAATTTGTTTTTTTTAAAATTTTATTTTAAAAAATGCC
GTTTGGTTTGGTTTAGGTTTCTCAAATCAAAAACAGAAAAATGAGCCAAACAGATTTTCTTTAATTTTTC
TATTAAGTATTTTCAATTTAATATTTTTCTATTAAGAAAAATATTGAGATCATGGAAGTTTGAACTCCGA
ATCTTGATGCTACACTTATATTTTAAATTAATTGGCGCACATATTACTCCAAATTTGACTTCAAAGCTCC
AAACGTCTTCTATGCCCCAAACGTAGAATTAAGGCTAGTCACATAGAAAGAAGCTGGTTACAAAACTGGG
TCTATAAAACCCCATTGCGCTTGTTTTGAATTTGTTGTGCCAATAAAAAAACACTCACTTACAAGGCTGT
AGACGCATATACTCTTATACGTACCATGGCCGCAAAAAAATGCAATACTCGTATTTTTGTGCCATTTCTC
CTTATTTTAGCTGCATGGGCAACAAAAATAGCTTGTCGTCCTCTTGATGAGCAGGAATAT
Sequence ID: 62
Sequence Length: 3000
Sequence Type: DNA
Organism: Poplar sp.
AAATACGACATCTCAATGTAAGTTTGTTTTGCTTTGAGTTGGTTTACATTTAACAATTTATGAACAAATA
ATATTTTATTAAATTTATCAATTTGCAGGTTCACAAACATTGAGGTTGCCCGAATAATAACATCAGCATT
TAAATCATCGATGGAAATTCTATTATTTCAATGAAGCCAGGTTTTCAAATATCCTGAATGGAAACTGCAG
GTCGATGCATAGTTTACAAGATTTAAGATTAGTTTTAATTTAAAATTTTTAGTATTTATTATGTTAATTC
ATTTACTTTATTGAAAAATTATTTATTTTTTTTTTTATAAATTTTTACTGCAAGGCTAAATTTGAGTGGG
ATAGAGCAAATGATAATGTAACGAGGAGGGTTTGAGAGAATTATGTTGTAACTAGGTAACATAAAAAATA
GTACGACTTTTTTTTTAATTTCACATATTAAAATTTCATTTGTCATTTACTCACATATAATATATTTGTA
TTATATAGGTTGCGTGATTTTTGGTATGAAGCAAATTTTTTTTTTTAAAAATATGCCAGAGAAAGCGCTT
TAGCAGGTTGGAATGACGTGACGATTGGAAAGGATTTTAGGCTGCCATACATCCTGGAGAATATATGGGT
GAAATATATTCAGCACGTGATGTCCGAGCGTTTCACACTATGCATACATTTTGGTGTGGGGAACCGGAGT
CGGTAAATTCACGGTTCGGTTACAACGCACACTAGCAGCTTCGTTCCATTTGTTTCACATGCGAAGCGTA
AGATTTATTTTAATCATATTTATTTTTAATTAATTTTTTGTTATTTATAAACATATTTAACTGAAAACAT
TTTTTTTCTTACAGGCTACGTCACTTAGAAGTGAGCTAAGCCCAATGGAGCTTTTTATAGAGACGCACGT
GCAGAGTGAAGACCGCCAAAAAAGGGTGTAGCAGTTCGTCGACAACCGAGCTCAACACTTTGCGGTACAT
TGGTTTTCAACCATTTTATTTCTTAAGTTATTATTTTCTTGAATTTGATGATTTCATTTTTTTATTTCTA
GGAGACATGTAATAGCCGGTTGAAGGAGAGATATGAGGACGATCCTTCGATCCACCTGGATTTTGATTTG
GATTTGTAATTGGAGGCAGGATTACCTGGTGGACCCGATAGAAATCGGGTGTACGGACTCTCCACCACTA
CGGTCGAGAACCTGCGGATGACCCATAATGTTTTAACCGTTGGGATAGAAATCGGGAGCTTGCAATTGCT
ATTGAGCACTCAGTCTCCAGAGTTCGCGGTCTTGTTAGATCAATGAGTGCAAGAACCATAGTTTTGAAAC
CCAACCCGGTGGTCGACCCGGTTTAATGATCGGGTCACGGGTCAGATGGGTTGACCCGGGTCAACAAAAA
AAATCAAATAAAAACTTTAATAAATACAATTCCCCTCTCTTATCCCTCTCTCTTCAATCGACTTTCCAAC
TTTCAAACCACTTTCCAGCTTTCAATTCATTTCCAGCTTTCCAATCACAAATAAACATTCTTTTTTAATC
AACAACAAATAGGCATTCTCTTTAAATTGAGAATAGAAGGTAAAGAGGAAAAGAAAATCTTAAAGAAAGA
GAGAGAGAGAGAGAAAACACATGGGCATCAAAACAGAAAACATCAGCAATCATGCATCAACGACAACGCT
GATATTCACGCACAAAACTTTGAAGAGGGGCTTTTCCAATCATGTCTTGATGCAGGATCTGATAAAAACC
GGGGATGTTGTCTTTAATGGAATCTACAGGAATGAGCAGGGATCACTACGAAGAGGATGCCGTGTTTGGG
GTAGAGGTGTATTACGGGCAGTCAGGCACAGGAGCTATGCGATTGAAATTGCTTTTTCTTGTTATCTCTG
TCTGCAATTTTGCATCCTTCATCCTTCATCTGCATCGTTTTGACTTTAAAAGGCCAAAACGAACGACGTC
GTTTCCTGGCTATGTATATATATATATATATAAAAAGAGACCGGGTCTTAGCCGGGTTTGTCTGGGTCGA
CCGGGTTTCGCCGGGTCAACTCCTCGATGGGTTTTTACTTAGACCCGGACCGGTCCTAGGCCTGGGTCGG
CCGCGTCGACCCGCCGGGCCGGTCCAGGTTTCGAAACTATGGCAAGAACATACAACCCATCTCAATGAAA
AATATAAACGACTCTCTACGGATTATAAAGAACTTGTGCACCCTTTTATTAGCCCTATGGTCCTGGGGAT
GACCAACCTCCTCTTTCCCCTCCAGCGTCGTCTTTGTTTTAGTTTTATTATATTTGAACAAATAAATTTG
TAATAAATATTTGAATAATTTATTTTTACATACTTGAATTTTATATAAATTTATTGAAAGTGTCTTTTTT
TAGTATTTTTTTTTTTATTCTTGTTGCTTCATATATATATATATATATATATATTTTATATATATATATA
GCCATAGGATCTTCAGTTGTAATTAAGTTGATTTTATTGGTTCTTAAACTTTGAAAAGGTAATACAACCT
CTTTTAATGTGAAGTTGTTAGAACAAATGCTTGAATCTTTGACAATAATGTTTATATTGCTTCGTCTAAA
TTTATTCAATAAATAAATATATCATATTTACTAATAGTTACAGAGAGACTACCATTTTCCTAGTTTTTTC
TCCGATGCAGTAACTTAAAGGAAGGCCGATTCTTGTTACAATCTGAAAGTTTATTTAAAACTATTCACTT
TGTGAATCAAACGGCACGGTAAGAGTATTTTTGTTTTCTTCTAAAATTAAAGATGGGACTAAATACTATA
CTCACAAGCCTAGCTAATATAAGGTGTCAAAATTAGAACATTTGTAAGGGCACGCATAGTTGGAAGGCCT
GTCTTGTGGAGCATACGAATTGACCGGAGCAGAGTCCCTATAAAAACCTCATAGGGTGCGTGAAGATAGT
ATGCCTACCAGCTTACTCATTGCTTCCCCAATATACCAGCTACTAATACCAAAGGCTGCC
Sequence ID: 63
Sequence Length: 3000
Sequence Type: DNA
Organism: Poplar sp.
AGTTTGGTCCTTGGTTCTGGATTTCTTCAATCAAGTCCCTAATTGGCCATCAAACTTCAATATTCATGCA
ATTAAGCCCCTGATTTGACCAAATCAACTCTTAAAATAGTAATTTGACCCCTGAATTTTAATTTCTTCTA
ATTAAAGCCCATATTGACTTAAAAAAATTTTTTTTCTTGCAATCAAACCCTTCATAAATTCAATTAAACC
TTCAATAAATTTAATTGAGTCCCTAAACATCTGATTTGGACTTTTCTTCATCAAATTGAATTTTCTTTGT
CAACAGGGCTCTCATCAATTAAAAATATACTGTCAAATTTTAATCTTTGTATTTTTGAACCTCCTCAACC
AATTTCTAGTCATTTCCTAAGCGCTCCAGCATCCTTTTCTTACTGCTATTATTTTTTGGATTTTTTGTTT
TTATTCCAAATATTTTTTTTGATTTTTTTAAGAGAAAAAGTGAATTTGGGAATAACTCAAAAATAGGTTA
TAACACCTTTTATGATCCTCTTGCTAGGACAATTGTAGATGTTGTATTTATCCTTAGAAAACTGACATTA
TGATTCTCATCACAAAACTGGCTTATGCTTAGTAGGTTACTTTAAGGTCATTCACAAATAAAACATTAGG
AAAAATAGGTAAACAATGAATTTCAACTTTTATTTTACCTTCAATGGTAGTTATGCTTCTATCTCCAAAG
GTCACTGAATCACCATTCGTGAATTCAAATTTCTTAAATTGACCTTTATCTCCTGACATATGTCTAGAAC
AACCACTATGCAGTGTGTACTACTAAACATATTTTCTTTTTCTTAACTACCTATTGATTTTTTATTTTGT
TTTGTTAGAACTTTCAGTCTCATTAGATTTCTCACAACCTACTGACTTATCAATTTTCCTTTCTATAGTA
TATAATTAATTTAAGATTAAGTCAACTTTGTATTCTAAATTTTACTTGCGAAACAGTTTCTTTTTACATG
TCCTTCAATTCTGGAACACTAATTAATAAGTAGGGATAAACTTACCTTTCAACTTACATTTTTGGAAGTT
TTTTGATTTGTCTTTTAGTATGAAAGGTTCCTTACCCTTTACAAAGGTAATCCCTTTCGAGGAAAAAGAT
ACAAATTTACTGTTGATGGAGATTTCATGTGTCATGTATCTTATTCCTGTACGATCAATACTTACTAATT
GAGCTTTCAACAATTCATCAAGTTTTTGTATTGCTAACGGTTCTTATGACTATTTGCACATAGTTAGCTT
GTTTTCAAATGCTTTGACTTTCCTAAAAGATCTAGATATCTTTACTTCTAACAACTTTAGGGCTTCTGCC
AATTTTTTCTTCTCATTCTTCATATCTGTCAGTTTATTGAATAATTTCTTGACCTTCACCAATTCTTCCC
ACAAGTCATCATAAGCTTCCTAAAAGCTTAGTTCATGATTGGCAATAACAAGAGTAATATCTAAATCACT
ATAATTACTGACAATCTGATCTATGTAAGAATCACACGATATCTCAATAGTTTGTTTCATGTCAATAAAT
CCGTTAAAAGTGTCAGAGAGAGCAACAAAAACAAATTCATTATCAGATGATGAATTAAAATTTTCAGAGT
TAGATTCATCACTTAAGGTGACATTAAGAGCTTTTCCTTTATTTCTTTTGAAATTAGGACAATCATTTCT
CAAGTGACCATATCCTAAACATTCAAAACACTTAACTATTTCCCTAGATTCAGTCTTAATTTTACTTTCA
TTTTCCGATTCATTTCTTTTGATAAAATCATTTACCTTTTCACCTTTATTAATTTTCTTCTTCTTAAACA
TAAATTTTCTGAATTTCTTAGCTATGAGAGTTATATCTTCATTATTAAGATTATCATTAGAAGAATAGGC
ATGTTTTTCCCTAGAAGATTTAAATGCAATGGACTTACTTTTTCTTTGATTAGGCAATGCAGATTCAATT
GTTTGTAAGGAACCTACTAGTATCAAGATCCTTGTTTTCTTCTAGGGATGTTACCTTTGGCTAAAATCTT
TCAGGTAAAGATCTTAAGATTTTCCTTACAATTCATGCATCCTCTACTTTGTCTTCTAAATTGAACTTGG
AATTCACAATTTCATTAAGTTTAGTATAAAACTCATCAAAAATTCATCTTCCAACATTTTAATTTTGGAC
AAAATTGTAAGACAAACTCGTGTTCTGAAAAAGCCAGTCTCAACTTTAGTTTCTACCTAGCTAGTATCAT
GTTGATCTATCAAAGTAAAAAACCACCTTTCAAGAAGGCAAATGCATGATAGCAACAGATTGATTTTGGC
AGAATAATCTTGCACGGAATAGTAATGTTATTTCACATACAACCTAACCTGGTCATTGACTATGCGGACT
CTGTATATTTTATTCCTCGTTCAAAAACAAAACGTTGGCTTCTTGAATCTAAACTCAAGAACCCATCAAA
ATCCATGTCCTAATACAGACCACCACGTTTACTAATAGCTACAGAGAGACTACGATTTTCCTAGTTTTTA
CTCCGATGCAGTAACTTAAAGGTAGGCCGACTCTTGTTACAATCTGAAAATTTCTTAAAACTATATATTC
ACTTTGTGAATCAAACGGCACGGTAAGAGTATTTTTGTCTGCTTCTAAAATTATAGGGCATCTGATGCCA
AAAGATGGGACTAAAATCACTGCACGAAAACCATTTATTAGCTTACTAAATACAATACTCATCAGCCATT
AGAAAATATACTCATGAAGGGAAAGTTTGAACTGTCTGAATTAATGCTGGCTAAAAAACACAACACTATG
TCAATTAAATTAGTTAATCATAGGGGTCAAAATTAGAACATTTTGAAGGGCACGCATAGTTGGAAGGCCT
GTCTTGTGGAGCATACGAATTGACAGGAGCAGAGTCCCTATAAAAACCTCAAAGGTTGCGTGAAGATAGT
GTGCCTACCAGCTTGCTCATTGCTTCCCTAATATACCAGCTACTAATACCATAGGCTGCC
Sequence ID: 64
Sequence Length: 3061
Sequence Type: DNA
Organism: Poplar sp.
TTTTTATAATGTAGATAAAGTCTTAAAATAAACACATTATTAAGGTAAGCATTTTTCATTCCTAAAGTCT
ATAGTTTTTTTTTTTAATTTGTTCAATATATTTTATTGTTTGTATTGCCATCATAATTGAATACTTTTTG
AATTTCAATTGTTATTGTTGATTTTATTTTTTTATATTTCTTTGTTTTAGCTATTGTTATTTTTGTTGTG
TTTTTTGTTTTGGTGTATTTTTATAATGTAGATAAAGTCTTGAAATAAACACATTATTAAGGTAAGCATT
TTTCATTACTAAAGTTTATAGGTTTTTTTTTTATTTTTTCAATATATTTTATTGTTTGTATTGCCATAAT
AATTGAATACTTTTTTTAATTTTAATTGTTATTGTTGAATTTACCTTGTAATTAGTAATTGAATATATAG
GAATAAATTTAATTATTTTATCTCAATTTTACTCTATTATTGCATGATATGTGCTTAATTATTTTCATTA
ATTTTAATTGTTATTTGTATAGATGTTAGGTTATATACAAGATTTTTGAATGGGGTCAAGTGGTTGTGGC
TATTGTTAATATTTGCAGGTTTGAAAACTTTGGATAGTCTCCAGTATAGGGGAGGTGCTGTCGAAATTTT
TTTTAACAATCGTATTTAATTGTATAATTATTTGTATAAAAGTGTGTAGATGCTTAGAACGAAATCAACA
GCTCGTCGCTCACATATGGTCGCAGCTAGTTCTTCTAGCAACGAGGATGACATGTCCTTAGGTGTCAATC
AAGAAGAGGTACCTGCGCCGCCTACTGATGTTGCCTCTTCTAGCATGGTTTCATAGCGCAAAGGTGGTGT
GCCTTCACAGCGGAATCAATTCACCCGCAAGTACGAGGCACAGTGGAAGGATGACCTTTCAATATAAGTT
TGTTTCGGTTATAGTTTTTTTTTTTAATTATAACATAATTTATGAAAAACTAATCAATATTTCATTAATT
TAATTTATTTTCAGGTTCACAAACATTGAGGCCGCCCGAGTACTATCATCGGCGTTTAAATCATTGATGG
AGATTTCATTATTTCAATGGAGTCGGGTATCCAGATATCATGAATGGATACCAAATATCGACGCATGGTT
TGACAAATTTGAGGTTGGTGTTAATTTCTAATTTCCAGCTTATTTTTAATAAATTATTAATATAATTGTA
AATAAATTATTATTTTTATTTTAAATAAATTATTTATACACAACACAAATTCGAGTGGGATAACGCGCTT
GACAATGTTGTGAGGAGGGTGTGGGAAAATCACGCGACAACTTGGTAACATCGAAAACAATACGATATTG
TTTTTTAAAAAATTATGTTTTAAAATCTAATTTGTCACTATGGAAGTAGGTTGCGTGATTTTTGGTATGA
AGCACAAAAAAAGGCAAAAAAAAACGCGAGAAATAACGATCTCCAAGGCTAAAACGACGTGGCGGTTTGG
AGGGATTTCAAACTGCTAGACATCCTGGGAGATATATGGCCACAATATATTGAGCACGTGATGTTTGAGC
GGTTCACACGATGCTCACAGTTCGGTGCTGGCAACCAGAACCGGCAAATTCATGGTTTGGTGACAATGCA
CACCGACGGCTTCGTTCCGTTTGCTGCACATGCGAAACGGATGGTAAGATTAATTTAAATGAAATATATC
ATTTAATTTGTTGTTACTTAATTAATTTTTTTTCTTACAGGCTACGTCTTTTGGATGTGAGTCGAGCCCA
ATGGAGCTGTTTGTGAGAGACGCACGTGCGAAGTCAAAACTGCCAAAAGTGGGTGCAACAGTTTGTTGAC
AACCGCGCTCAACACTTTGTGGTATGTTCGTTCAACCATTTTATTTCGTAAGTTATTATTTTTATTGAAT
TAAATATGATGATTTCTTTTTTCATTTTCAGGAGACCTATAATAGTCAGTTGAGGGAGAGATATGGGGAT
ATGATCCTTCGACCCATCCGGATTTCGATCCGGATTTGTGGATGGAGGTAGGATCGTCTGGTGGACCCGA
TAAAATTCGGTTGTACGGGCTCTCCAACACTACGGCCGAGAACTTGCGGGCGGCTCTTAGTGTCTCAGCC
GTTGGGAGCTCCCAATCAGTATCGAGCAATTTATCAAAGGAGTTCGTGGCTTTGCAGCAACACACAGCTC
ATCTCACCGAAAAATACAAGCAACTATTGGCGAATTATGAACATAACATAGCTCACACAGCTCATCTCAC
CGAGAAATATGAGCAACTCTCAGCGAATTATGAACAACTTTGCTAAATGGTCATGATCATAACATCACAG
AGTGGTGATACATGTGCGACCCCTTTTTGGTTGTATGGTCCCGGGAACAACCAGCCTCCTCCTCCAGCTC
CGTCATTGTGCTAATTTAATATTTTTTTGGAAACACATTCAATTTTTTTTATTTAACATTTAATTTTTTT
TATTTCCAATGGCATCAGACTACCATTTTTTTAGTTTTTTCTCCGATGCAGTATTAGGCCGACTCTTGTT
ACAATCTGAAAGTTTATTTAACGTATGCAAACTATTCACTTTGTGAATCTAACGGCACGGCAAGAGTATT
TCTGTCTGCTTCTAAAATTATAGGGCATCTGATGCCATAAGATGGGACTAAAATCACTGCACGAAAACCA
TTTATTAGCTTACTAAATACAATACTCATCAGCCATTAGAAAATATACTCATGAAGGGAAAGTTTGAACT
GTCTGAATTCATGCTGGCTAAAAAACACAACACTATGTCAATTAAATTAGTTAATCATAGGGGTCAAAAT
TAGAACACTTTGAAGGGCACGCATAGTTGGAAGGCCTGTCTTGTGGAGCATACGAATTGACAGGAGCAGA
GTCCCTATAAAAACCTCAAAGGGTGCGTGAAGATAGTATGCCTACCAGCATACTCATTGCTTCCCTAATA
TACCTGCTACTAATACCAAAGGCTGCCATGAGATTCACTAAACAATTCCAATTTGGTTGTTTGGCCTTGC
TCTTCATTTTGGGAGCTTGGCCTTCTAAATCCACAGCTCGAACCCTCCTGG
Sequence ID: 65
Sequence Length: 3000
Sequence Type: DNA
Organism: Poplar sp.
TGACGAAACCCAAACTCCTAAAGAAATTTAGATTCAACAATCCAAAATAATATATATAGATTCAAGAAGT
AATAAAAAATAAAAAATACGAATATGAAACTATGAATAGCAAAATGAATTTGTTCTAAATATAAATGACA
CGGAGATACAATTTTTTTTTTTATGAGAATAAGAAAACTAATGAAATAAAACTGTTATGGTCTCCTCAAT
ATGATGTGGCATGCATTCATAGACATCAAATCACACCAAACACTATTAAATTTATTTTGTCCCATGGAAA
AAGCTAGAAACAAGGTAGTGTTTATCAATAATTACCTTATTGTCTTTATTCAACGACATCAACTTGTAAG
GCGAAGTATAACTTTCAGTTGATAAATGAAGTTTCCTCATTGCCTCTGTAGACATCACATTCTAACAACT
ATCACTATATATTATTTGGCTATAAAATTTATTTATTTTATGGTGTATATATAATGAAAAATATTAGTAC
ATCATCAATCTTCTTCTGACTCACCCTTGGGTATCAACAACTCTTCTTGATAACAAAGGTAGTACCTCTA
TCTCCCAGTAAAATCTCCCCATCATTATTTGATCCATCGTTGTAGGCATGATTAGTTTTGTCCATAGGCT
TCTCTATCAACAAATTCCTTCCTAGATGACTACATGTTTTGTTGTACTCGGTAGCTCGATGCCCTAGTTC
ATCATATCTAAAGTAATGAATTCTAAAACCATCATTAATTGTCAATTCAACTTCGATCTAACACCCTTAC
CTCTTAATTCATTCTTAAAATTCTTAAGTTTGGACTTAATTGTCAATCTAAATTTTTCCCTAGGTTGCTA
ATCTTTAATTGATGATTTCCCCTTCTATAATGGTTAGCTAGTTTGTGGTGACAAAAATGACTTATACTCA
TTAAACAATTCATATAATGAATCTCTAATTTTGAGTAACATTTTCATAATACCATCATCACCATAAAAAA
AAAAAACTCTAGATACTATAATGTACGCCTAAGATCAAGAACAACCACAACTACAACATAAACAAGTATG
TTCATTTTATTAGTGCCTCCCTTATTCTTATCAAATCTTTCTTTCATATTATCGCCATCAAACACACACA
CAAATGTCTTCATGTTGCCTCAACTTGCAAAACACTAATCATCGCATTAACTTCATTAAAAAAAGGTAAA
TAAGAAATTAGAGATTTAATTTGAACACAATTTAATGGATCACAAGTTGATACATAAGGTTAATAAACTG
ATTTGATGAGTCAGTATTATGTATTTATAAATAAAAAAAATGTATCTATAGCAAACTAACAATATTGGTG
TTTATGAAGAAAAGAGAGATCATGTATTCGAATTCCATCAATTACACGAATTCAAACTTAGTATTGATCA
GACCATTCAGGTTTGATTGAATCGGATTAAATCTTTGTTTAAGTTAACTTTAAACCTAATCTAAGTTAAA
TTATGTGTTACTAAATTTCTAGATTAATTTGTTGAATTGTGTCGAATATAAAAACCATGCTCTCAAGACA
TCTCATAATCGAGTTTGAAGACTATGATTGGGTTAACCTAAATTATAAGCTTGACATCTCCAGTTCCACC
TGATTAATTTAAGAAGCCCATGGACCTTTAATGCATGAAAATTATATTTTGCTTATTGAATCCAAACTAA
ATACCGAACTGACAAAAAAAAAAGTTATTTCTGTTGTCTCCGTTCTAGCAACAAAGAGATGGGTGCGATT
ATATATATATATGGAGTACGCTGCCATTTCTTTCAGTAATTTCTTGGTTGCAATGCGAAAGCACTATGAA
ATTGTTAAGAAAATGTAGAGGAAGGTGCATTCTCCTTTCTTGATTCAAGATTATAGAAGTTATTATTCTT
ATTATACAGTAACCTAAAATAAACAATTCATTTGGTGAATCAATCGGCACGGTGAGAGCATTTGAAGCCA
AAATATTGGACAAAACTCATGCATTAGTTTAGCACATACACCACTTTATATATAGTAAAGCACATTCAAA
AGCAAACTATGATATGACAACCTAAAATAAACTCTGCCTTGGAGTGTGTTTATATATTCAAGTCTTCAAA
TATCTTTACGGAGAAGGAGGACATGTTATTAATTAGCGAAGAAATTAGGATGTCACCATTGGGTAGCCAT
GATGGATGTTAATTGTTGTGTCATGGCATTATTCTTATGCCATGTATTGATAAAAATATTGGTAATGAGT
AGTATAATTGACGTAAAAAATAGTGTATTGATAGAAATATTGGTGATGAGTAGAAAAAATAATTTGAAAT
TTTATATGTTTGGCTTCTAAGAACCCACAAAAAATACAAAAAAAAGAATGTGTCAAGACAACATTACACG
ATCATGAGTAGAAAATTCCTGCCATTTGGGCAGCTGAAAAATTTTTATTAAAAAAAAGCTTAGCAACATC
ACATGTCTTAAATCTAGACTCGATGCCTAAAAATCAGTGTATGTTTCTTGTTTCAAAGTGTTTATCTTGA
ACCTTAGTCACAGTGAACTAGTAGTACAATAACATGTCTCGAAGTCCTACCTCTCAGAGACAATTGCACT
AAAATGTTTGCTGATTGCTTTAGTTGATCTGATGCAAACCATTCCCATGATTTGAAGAAACGGCACGGTA
AAAGTGTCTTTGTTTGCTTCTGCTTGTAGATCATTTCATGCCAAAGAATAGGACGAAAATCCCCTGCTAA
ACCATTTTCTGGTTTACTTCATGCTCCATTCATAAGCCATACAAAAAAGAAGTCGGAATTAAAGGGAAAC
ATTTCGAACATCATCGGGGGTAAGAATCACAACATTTTCAAGGGCACGCAGACTTGAAAGGCAAGCCTCG
AAATATTATAAGCACAACGGTATGAGCAGATGCACTATATAAACCTCAAAAGATGCAAAGAGATATGTGT
GCTTGCCTGCTTTCTAATTCCTTCACTACTATAGTAGCTGCTACCACTGCCAAGTCTATC
Sequence ID: 66
Sequence Length: 3000
Sequence Type: DNA
Organism: Poplar sp.
ATCTACAAGCCAAAACATATGAGTCAGCCATTTTGAAGTGCTGCATAAAAGAAATCCAACTTCTACTAGC
TATCAATTTAGTGGAACATAAAAGTTCAAGCTGAGAATATAAATAAGATACATGTGCAATTGCAAATACA
TCATCAAACATAATAAGAAGTGTAAAGCTCAAGCTAACAAACATAAAAAAACCCAACAACTTTGGTTTGG
ATTCACCTTTGAGATGAATGTTTTGCTCGTATTCATCATAGAAAAAATAAAGCTATAGGTTGATGGTCCT
TTCAAATTTCCTAAGATAGTGGGAGAAAATACATATAAATTGAGGTATTAAAAATTTATGATATTTTAAA
AACTTTCAACATAATAGATTTCTCTCTCTTTCTATAATGGTAACATAGTTGAATAAAACTAGGAGACAAG
TTATTGCACAGCAGAAGTGTATGACATCAAATTAAGACTTGCATATTAAAAAAGTTTATGAATCATTGAG
TTAATATATGATGCATTTATCTTATTTATTTTCAAGTCTAGATAAAGAGTCTTTTAGTCTATGTGATGAG
TGAGGTTCATGTATATGAATTGAGTTGATATATGATTCATTTATCTCATTTATTTCAAATTGATATCAAA
AGTCTTTTAGTCTATGTAATGAATGAGGTTCATGTATGTTATTAAACCTATTCATGGTGGGTTGTTTATG
CTTAGTTAATAAATACTTCATTCCTCCTCGTTAAAGTAAGACATCTTATAATAAAAATAAATAGTTTATT
CTAATAAAAAGTGATTCTTCCATAATTATCTTATTTCATCTCTTTGGTGGGTTCCTTTAAGTGATGAGTT
GATTATTTTTTAGAGCTTACTTGTGTTTTCTCTTTTAAATCTTCATTAGTTCCAAGTCGATTATCATCGG
GATATAGTTTAACTAATTATTTTGATTTATATAATTGTAATGTGTGGTTCAATAAAAAATTGTTTTGGCA
CATTGTTTGCATAATAACATTATAGATGTTGTATACTAACATTGGTTATGAAAAATATATTAGAAAAGGA
CACGTTCCTTGTCTATCAAGCTTTAAAGATCTTATACATCACAATAAATAATTGAATAAATAAAATAAAT
TTAATACATAAACATATCCTCAAGCATTGTAAAGAAAAGTTGAGAAGATATCACATCACAAGAAATTTCA
ATTGAGATTAATAATCAACATAAAGCCTGTAGATTTTTATCCCCTCATAGTAGATTTTGAGCTAGAACAA
TTAATGTTTAATAACGATTCAAAATCTTTAACTACTGATTTAGCATGAGCATATATACATGGTTGACTTG
AAAATGCATTTGATATAGGACAAAGTGAAAATTAAAAGAAATAAATTTGGAGAAAGAATAAGCTTAGAAT
TAAAGGAAATGAGCTTAAGAAAAGAAATAAAAGAGTTAAGGTAAAGAGGAAAATGGGGGGATAGAAATGT
TTCCTCCTCAGTAAATTTTTATTAAAATTCTAATTAAAATAAATAATTGGATTTACAGTAAACTAAATAA
AATATTCAATAATAATTAAATCAAACTTAACAAATAAATCCAAATAAAATTCAATATAACCCATTAAATA
AAATATTTAAAAATAACAAAATTAAGCTCAATAAATAATCCCAAATATAATTCAATAACATAAAGGTTTG
ATTATCTTCCATCATCATAGATTATATGGATATATGAACATGTCTCGAATTTGATGTTACTATCAACACC
TATTTATCTTGAACCGATTTTCTTCTACAAACTCCCGTGTGAACCACGTCCATTGTGAAAGGATATGCAA
GCTGCACAATTCGTTAAGAGAGTTGTAGATTCGAAAGGATCCCACTTGCAGACAATGAGTAAATGATAAA
TCTCTCTCTTCTGTTTTTTTTTTTCCTAATAAGCACGACGATGCCATTAATTGACACTTAGCTTAATGAG
CTAGCAAGAAATAGGGACACGGATTATTCTCTTGAATCCATCTTCATTCTGTCTATAGCTCCACTACTGG
AGTAAAGGGAAACCATCTTACCTCTCTTGCTTTCTAGTTTTTATTTAATTAATCATTTCCAAAATCAACA
AAAAGATTTCTTTGCACGTTTGGTATATGTAACAACCAAAAACTCAGGTCAAGGAACAATTGAAGAAAAT
TATGTTTTGAAGAATAAGCTCAGGTCAAGGGATTCCAATGTGTAGCTTATGTTTAAATTTTATGATTGTG
CATCTGCATTAGTAATGATGAAAAGTTTCAATCAGAAAAAACCTGCATTTTCTTTTCCACAAAGTCTTGT
CGATCCCTGCATGTCGTGCGAACCAAACTGGTTTCAGGATAAAGTCCTACGCGTACCTCTTCAAGGAACT
TGTTTCGGCTGGCATAGGAAAATGGAACTAGCAAAAAAAGGTAGATTGCTAAAATGTTTAAATTCAATAT
GTCAAGGGGTACTCTTTCTGAAAGAGATGACACATTAAGTACGGCATATAGGCTCAGGCTCACTGATTAT
TATTGTTATTTTGCTTGAATTCATGCTTAACACTCCAATATTGAACGTAACTTGAACAATTAGTTGGATA
ACTCACAAGTAAAAGGAAAAATAAGCATTGCAGAACAATACAAGTGCTACAAAATAAAATATGAGAAGTG
CAGACAACGCAGCAGCGTCCAAATTTTGTGTCAGGATCATACACAACGTGCCAGATTCACCAGGTTATTT
GCTTCCACATTGTCAGAATTGAAGCCATGGTGTGAATGCTGTCTGAGAGAGATCTTTAAGCAGACCAATG
CAAGTACTATAAATACATGTAATTCACGAATGACTAGTGCAATATCATCACATCCTACAATACTTGCCTA
TAGTCACAATCTTGCACACTTCAGCAGAATGGCTTTTATACCAGAAAACAAACTAATCTTTGCAGTGGTC
TTACTTCTTGGGCTCTGGACTTCTCATGTCTGGTCACGCTCTCTGTATGAGGCTTCCATG
Sequence ID: 67
Sequence Length: 3000
Sequence Type: DNA
Organism: Poplar sp.
GGCAAAAACAAGCCTTACAAGGTAACCTCTCCACTCCATTTACTTAATCAATCATTTACATTCTTGCTCT
CTCTCTCCCCGTAAATTATTTCTCTCAACATTCATGTTTAGAAGAAGATTCGGGATGTTTTTAGTTCAAG
AAAGAGGTCAGTGAGGAAGGAATATGAGCAGCTTGCTGTACAGTTCAGAAGTGATCCAAAATCCAATCAA
GAAGAGAGTAAAACAAGTTTAATGGCAACTCCGTCTATAAAGGAAGCAAAAAGATCCTCATCTCATGATC
CCTCTGAATCTGAATGGGAGCTTGTCTAGATACCAGCAGTGTTTAGTATTAGTAACACGTCCGTACGTGT
ATATAAATGTTGAATGGGGTGATTAGGATGTTGTGCCAGATGTTAATATTTCTCCCAACCTTTATTCCCC
TTCTTTGCATACTTTCTTCTCTTCCCGAGTGCTGAAGTCGGTAAACGGCAGAGAAAAAAAAACCACCCTG
ATCTATAATGCAGAAATTTGTGAAGTTATATTCAGTTATTTTATGATGTCCTCACATCATGGGGACACAA
GTATTTACCTTGTATGACCTGTGAGTATTGTGCATGCCTTGAGTTCCAAGCCAAACCACTTGAAGGAATT
GATTATTTCCTTCCTCCATGAAGTTTCAGTTGTTTTAAGAGCTGCAAGATTTGCTACATTTGAATTTCTT
GAAATTTGTTGTTAGTAATTCTAAAACCAAGGCACACTGCAACTCTGAAGGATACGTAGATGATTGTAGG
CAGCTGATGAAGATAGGGCAGACAATAACAAGTTAACTGATCTGATAATAGTAAACCAAGGCACATCACA
ACTCTTGCTGAAGGTCAAACAACCAAATGTGAGGAAATTGTAGTGTCCAAGACCAAGGATACATGCAAGA
GCTTGTTCCATGTATCACGATGAGCCTTTGATTTTTCTGTTGCAGATCAGTTTTCAAAAGCTTGTGCTGC
TTTCTTGCATACAAAGCTGGATGTGCATCGCTTCTATACTCTTGATAGCTTGGTATTGTTCAGGTATCTC
ACTGGTATCATCATGCTTCCAATTGTTTTCTCAATAATTTCTTTTGCTGGGTCAGGAAATGCACACATAG
GATTCAACCATGATGGGTTGTGTTTTATTGCAACACTGGTGTTGTGGTGATATGCCACAGAAAACGATGG
TTGTTTTGGTCGCATCAAGGTGGCGAATGATCCAACAAGGCCATGTCCTATGCCTTTATTGAATGCAGAT
TCATTCATTTCCTCTGCTAATTTTCCTTTGTATTGGAACAAATCCCACCTGCAAAAGTTGACATCCACTT
CGACATTATTTGTCTTTCAAATTCCACAAGTTGACTAACCTTGTCATTATAGTTTTACACGAAACTTATG
GCTTGAGCTCCCCACTGTAAACCCACCAAGTCATGGCCATGGTGAAAACCATGGCATCGTCTCCCCGCCA
CATTTGGGCCAAGGGCGACAGTTTGTCAAGAAGCCTTAGAACTCTGCTCCCGTTTCTTGCTTGTGTTTCC
CTAAAATGGGCTCAACAAAACTCCGGTAACAGGAGTAAAATCCTATGTTTCAAGTTTTTCTTAAGTTGAT
TCATGGTTATAATTCAACTAATAGAATCACTAGAGGATTAGAAACAGAGTAAAATATTGTAATCTTACCT
TTGCTTTGAAGACCTTATAGACACAGCCTCCGTAAATATGAGCTCAAGAAGAAGGTATGGCAGAATGAAG
AAGGCAAGCTAGAGGTTCCCATTGGTTTCTGTTGAGGGAATTTCCAACTATTATCACCATCTTTCATCTT
AAGTCTCTCCAACATGTCCATGCCATTAAACATTGAATACAAATCTTTGTTAAAAAGAACACTAAACATG
CACTTGTTTATATATATATATAAGCTGAAGGACTGAAAACACTTGTTTTTAATAAGCCAGAAAATTGGCT
TAAAACACCTATCATCAAGGAGAAAAACTAAAAGAGTGAAAAAAAGAACAGAAGGCCTAAAACAAAAGGA
AAGCATACCAATGCTTGTAGATAACAATTTAAGCATCAGATTGCAGCAAACAATTAACCAAAGTATAAAC
ACATCTAAATGATAAATTAAATCTTTTTAGTATAGTGTTTAACATTCTCAGGAAAAGAAAACATTAATAA
GTTCGGTTGAAGAGATAAATTACAACCGATACTCCAAACTTTTAATGCTCTAAACCCACAACACTTTTAA
TACTCCTAGGATAGCATTTGATTATTGCTTCGAAATAAAAACAAAGGGGACAAATGAGGCATGCATCTCT
TATATTTCATGTTTTAAAATTATATAAACGTATTTTAAAACTATCTTATAGATGGATTTATTTTCAAAGT
AAAAGAAATTATTTTTTTTAATGCTACCCTATGAAAGAAATTACAACACTTTTAAGGCGAGTCATTTGTC
CTCTATGATGGCCAAGAAAAGGAAGAAATTAGTATTTTGATTGCATTTATTATCTACTACGAGGATTGAA
GATCAGTGTAATATTGATGACTTCCAGACATGCTTCACAACTATTTTTAGACACTTCAATTGAAGTTAGT
ATAAGCTGCCTATGAATTTGAACACAGTTAACAATATTAGAGGCAGCATGATCCTGCCAAGACTAAAGAC
TACAATTACTGTCAATTCAAATATTAAATAATCAAAAATCATTTCCTTTAGGTTATATTTTTTGTTTTGT
ACAATAAAAACACATTTAGATAGTTGAAAAAATATTTAGCCATATATCAGAGCAGACTGCACGGTACACT
ACTACCAGGCCATCTAAATCAAATGCAATTTGCCGGTTGATCTAAGCATGGGAGGTGTCTTCATCTTCCT
AAACAAGTGCATATAGATTCATAAAGCAATCAAACAGGAACATTAGCATACTATATATAGAGGTTTTCTG
CATATGTGCAGCTCATTCATTTATAGTTCAAGCAGATTAACTACATCATCTAGATAAACC
Sequence ID: 68
Sequence Length: 3000
Sequence Type: DNA
Organism: Poplar sp.
TATTCCAAACAAAAGAATTCATTTAGCAAATCAAATCAATTCAATCTACTTAAAGAGATGAAATTAATTC
TAGTATAGAATTGGTTCCAAAGAGCTGTTAATGTTTGGTCTTAGTGTCCATGTGATTTTTTTCCCCTCAT
AATTTACAAAGTTGCTCACAGATATAGATCTCTATGACATTGAAAAATGGAGATTAGGATAAGGTTTGAT
GAAAAACATATTTTACCTCCATAATAACTTTCAGAATTCTTAATCCTAGTTCGAGATGCTGCTGAGTTAA
ACTAGGATGATAAAAATGAAAAGTATCCAAGTACTACCATCTCCATAGCTGGTTGGGTGGTAAGAACACC
TGCCCTGCTCATTTTTCGAACTAGTCTGGGTTAAGTAAATGGATCCTTTGCCTCTATTAAGTTCTATTTT
CAAGCAAACCTTCTGCAAACTGATGTCATATCAAATCTTTTAACACATCAAATGGAAAATCTTTTGGTCT
ACTTCCACCCTTCCTAACTCCTACTTCAATCTGATCTATACACTTTCTCCCCAATGCACCCTTGGACTTA
TTTTGAACATGAACAAAACTATAACATTCGACCTCCCATAAGTTGAGTTTAACATGCATCGAATTTGCTT
CTAGATTTCTTAGATACAAAAACTCATCACTCAGTGATTAAAACATATGTGATTTATAGGCCGAAACTCA
TACCTGATACCCCTACTACTTCCTCCCCAGTCACAGTAGCTCACAACCAGCTTTTTCAGCTGCCATATAC
CTTTCCAAGCCATCTGACATTAGAAAACAATGAGCTAACAACAAAACTTACACAATAAAAACGCAAAATT
TTCTCATTAACAAATGAAAAATCTCTTTATCTTAAAGCATACAAAGTTACAAAAAACAAAACCAAACAAG
CAAGTGACGTGATTATTTCCCATCGAAACAAACACAAATTCCTAAGCCACTCCAATCCAAAATCAGCTTA
AAATCAAAAAATCCACATGAACAATGACATTTCCTCACTTCTACAAACACTCCAGAGTTATCATCTCTGA
ATACTAATCTGCTCTTCAAATGAAAGAAATAGAAAAGGGTCTCTTCAAATTATTAAAGCCGCCTTACACC
TTCATTAATTTGCACAACAAAATCACATAACAAGCAAAAGAAGAAAAAGGGTCTGTTAAAAACACAATCT
TTCAATCAATAAACAGACAGAAAGTCACTAAAAACCCAACATTACCATTCAATTAATAAAACCCATTAAA
TAAACAGAAAAAGATCAGATCTTTTGCTCTTTTTGCAGAAAAAAAATCCAAAAAGAAGCCAACTTCAGAA
AACCCATAAATCCAAAGAGAGCTACAAGGGTGAAAAAGACAGGTACTCAAAATGAAGCTTTGAGAGAAAC
TGACCTTAAGCTTTGTTGATGGGGCTGCAAGGGTTTTGCGGAGAAGAGGGACAGGGCGCTGCTTAGTGAA
GGATTTGTGGGGTTTTGGTGGCAGGCTGATTACAAAAACTTTATTTTTACCCCCATAGGCTTTTAATGTT
TCTCTTCTATCCTTATGGTCTACAATTTTCTAATGCTACTCCTATTGTTTCTAAGACGTTTCAATTTCAC
CCCTAAACATCAAATAGCTAATGAAATCTATCGATGAACCTACATTTGATTTTTTTCAACTTTCTTGTGA
TTTTAGATTGATTCGAAAACGAACCCCAAGTCATGAGCTCATTAAGTTAACTTGTACGTCACTAAATTTT
TAAGTTAATTTTTAGGTCACCGAGTTAATTTATATTAATTTTTTTATTATATAAAATTTTTAGATTAATT
CTTCGATAATTCTTACGGTAATCTTGACAAGTGTAGAGTGAAAATCAATTCATCCAATAATATAAGAGAA
GACAGTGGAGACATGGACGTGAACTCCAAATTAAAGATGAGATTTTCTGCTGGTAGCCCATTCTGGATTT
CCAGGTTTCACTGCATCTCAGCATGGCTAACTAAAAACGATGGCAAGTCTTTGCCAGATAAAGTTGTTCA
AAGAATTGTGAAAAAACTCCAGGATTGCAAAGTCGGCTGTGGTGGACTTCAAAGGATATATTTCGAAACA
TTTCTCCATTGTCTGTTCAGGCTAGGATGGAGTACCTGCAATTAGCCTCCTTGGAGGGCAAGAAACCTTT
GAATTGAGTAAAATTCTAGTGGGATGTGTCTGATCATAATGCAAAGGTTACATGAAATGCCCTTGGAATT
GCTAGGAATGTACGTACCCTATGATAAATGCCTCAAAATTCTGCGAATTTCAATGTATTTCCATCGCAAG
GTATCCGAGATGCTAAACACATCAACTTTCAGACAAGGTTTATGTTGAAGGGCCGAGCTTCTTTTACGTT
GCTCGAGTAACTGTAATGCAGTAGTATCTTTCATTTGAAGAATGAAAGGAACTGCAACATTTTCAGGGCA
AGTCATTTGTCTTCTATGATGGCCAAGAACAGGAAGAAGGAGGACTTTGGCTGCATTTATTATCTAGCTT
GAGGAGTGAAGATCAGTGTAAATATCAATGACATCCAGCCATGATTCTCAGCTATTTTTAGATACTTCAA
CTGAAGTTAGCATAAGCTGCCTGCGAATTTGAACACGGTTAACAAGCCTAGAGACAGCAGGATCCTGCCA
AGAATGAAGCCTCCAATTACTGTCAAGTCAATTATTAAATACTCAAAAAACATTTCCTTTAGGTTCTTTT
TTGTTTTGTACCATAAAAACACATTTAGATAGTTGAGAAGATATTTAGCCATGAATCAGAGCAGATTGCA
CGGGACACTACTACCTGGCCATCCAAATCAAATGCAATTTTCCGGTTACTCTAAGCATCCTGATCTTCCT
AAACAAGTGCTTATAGATTAATAAAGCAATCAAATAGGAACATTAGCATACTATATATAGAGGTTTGCTG
CATATGTGCAGCTCATTCATTTATAGTTAATACAGTTTAACTACATTTTATAAATAAACC
Sequence ID: 69
Sequence Length: 3000
Sequence Type: DNA
Organism: Poplar sp.
TGTAGCCACTAGTTGCAACAATTTTCTACCTTGCTCTTCTTGTTCTTGGCAGCAACCAGTTGCATTTTCG
CTTGAAAAATTGCAGGAACAAGACAAAAACTCCGAGCAACTTTTTGTTTGGAAGCAATCTGAACATCAAG
ATATGAACTTCTTTTCTTTTGAAATCTTACAAGCCCTACATAAACCTCCATCAAAACTACAAAAGAATTA
AAACCCCAAAATCCATCAGATCTTGTCAGACAAAAGAACCCATAGAAGAAATTGCTCTGTTTTTTTTTTT
TTTTTCCTGATACCAAGGTTTTCATGACATATCTCATAACTCTGAATTCCTGTTGATCTCCAAAACGTCA
GGCAACTCATTATACATCTTTTGACATTCCACATCAATCTCAAATGTTCCATTATTATAAACCCCATTGT
TCCCAGCTTCCGGAATTGTATGACCCATTATCATCCCCTTCATCCCAGCCCCATAACCACTGCATTCCTC
CCTCAACAGTATCCTGGTGCCAATTTTCCTCACAACTGCGTTGTCAGATTCTGCAATGCAGCTATCCTGG
CTCTAATACCACAAAAGAACGGGTTTAATACCAAGAAAGTCAAAATGAATCCATCAAATACATCCTCTTG
CTTTCCTAGTCCAACACCCTCACTCTTCATTTCATTACTTAATCTGCTGAACAATACCAATAAGCTAAAT
TCTCTACCTTCTCTAACCCTGTATTAATCACATTCCTCAAGTGGAAATGTATTCTACGAAAAAAATATTA
AAGGGCACCACCACACGAACATTATAAAGATAACTTTGATGAGGATGTCCAACATAGAAGATCAGTACAG
CATGGGATAGCAGAGTAGGAAAAACAAATAGTGATCCCAAGCTTAATAGAATCAGTATAAAGGGATTCAA
TTAATAATTATTCAATATAAAAGGTAGTTGTTAAACAAGAAAATTAAAAATAAAAACTAGTTTCTGGATA
ATTAAAAACAGTATAGATCAAATATTTTCCATCAGAACGTCCACACTAGCAGCGCTCCTTGACCACCTGC
AATAAGTTCCTCTTTTTCGCATGTTCCAATGCAGCATTACGCATCCCAAATCTAAACCTCCATTTCAGCT
ACTGCAGTGACAGTGGATGTGTTTTTGTCCACGGTATTTGACATCGAACGCTTAGTTATCGGCAGGCACC
ATCTTGTGCATATTTCCAATAGTAGCGTGACCAGTAGCAAAGTTAGTATTATACCACTGTAGGTAAGCCT
CCAAAATCTTGCGGCGTGCAATACGTCAAATCCTTTGTATATATTGAATATGCTTAGGATAAGTGTTGAG
CAACCCGCCAGGAAGTGGAACCAGTTGAAGAAGGGACGATACTTGCTATCTTTGTCAGGTCTGACCAGAC
CACCAAACACCTGCGCTGTTGCAAGACAGAAGAGGACAATGCCGATGATTTTGTGACAACTATGTTCAAT
TCCATGAGACCTTATGCCAAGAAATATTCCAGTCCCAAATCTAGACAGACCACCAAGAATATAGGCCAGA
AGTTGACAACTGACATGCAAGTAGAACCATAAAGGGCCTGCAGATTCGAACCTTTTCAAGTATCTTGCAA
TGACGGCCCCAACAGGCATTAGAATACCCCAGCTTACTGTGTTCAATATTCCATGGACATTTTTCAGTGT
TGTTGAAGAACTTCTTGTCGCTTTAATCCTCCCTGAAAGGAAGTCCAAACTCCCCATGGATTTGACGTTG
GGACTGGATGGAGCAACACTATGCATCATTGGAGTGTTTTCAGACATTGGGCCTACTTGCCAGAGATGAT
TGACTGTGCTGATGTTTCCATGGAGCTCTAAGCTTGCATAGATTATCATCTCATTGTTCTGATTAGTAGC
GGACAAGTCCAAGACAGGAAAGCTAAGATTTCCTTGTTCCAATCGCGTTCCATAACTTGTTATAGGCGAA
GTATAGACCGCAAGACTTCCATCCGTTCTCTGAAAAGAAACGATTGCTTGGGAACCAATCATCCCTCCTG
AAGTTGGATTGATAGCCCAAGCAATCCATCTGCGATCGGTCACGCCAGTATGCCTGAAGGCAACCTCAAC
TCTGCTGGATAATGGATGGTAGTTCCAGTGAAGAGAGGAACTCAAAACAGGCAAGTCGCTGCAAGAAGAG
AACTGTTTGTTGTTTGAAAATTTATAGGTCGTGCATGGTTGTTGTTGTGCTATGCATGACAATGTGCAGA
AAGAAAGAAAGATGGCGACCAGAAAAATCACAGAACGGGCCATGTTTATGCAACAAAAGGGCTTGAAAGA
AGAAAGATCGGTGATGACTAGAGACTACGGAGCACGGGGAAGGAATTGTTTTTGGATAGAGAATTGTAAG
GCTGATTGAGTGAATATATAGGGATCTTGTCTTCCGTTTGCGACTCGGAAAAATATTTTGATTGCTTCGG
TTTCTTGAAGGAAGTAATGTCCGTCTTATAGTCTGAAAAGCAATAGAGAGATATTTCAGTAGTCGGTCTT
TCCTTTTTTTTCCAATCGGATTAGCAATTTATTATTTTAAATAGTTTTTTTTTTTTTACTTTGCCTCAAT
AAAGGAAAATATTTAGCCATATATCAGAGCAGACTGCACGGTACACTACTACCAGGCCATCTAAATCAAA
TGCAATTTGCCGGTTGATCTAAGCACGGGAGGTGTCTTCATCTTCCTAAACAAGTGCACATAGATTCATA
AAGCAATCAAACACGAACATTAGCATACTATATATAGAGGTTTTCTGCGTATGTGCAGCTCATTCATTTC
TAGTTATAGCAGATTAACTACATCATCTAGATAAACCATGGTTTCGATATGCAAAAGGAATCAAACACGA
ACATTAGCATACTATATATAGAGGTTTTCTGCGTATGTGCAGCTCATTCATTTCTAGTTCTAGCAGATTA
ACTACATCATCTAGATAAACCATGGTTTCGATATGCAAAAGGCTATGTTTCTTCGCATTC
Sequence ID: 70
Sequence Length: 3000
Sequence Type: DNA
Organism: Poplar sp.
ATACAAAAACAATTTCAATTTCTCAACAGGGCAATGAGTCAGATGAGGTGCTCGTGAAGGCATCATTTCT
TGCTGAATTACAGCGACGTGTTCTCAAAGCTGAAGCTGCTCTTAGAGAGAAAGAGGAGGAGAATGACGTC
CTTCACCAACGGCTCCAACAGTATGAGAACCGTTGGTCAGAATATGAGTTGAAAATGAAGTCCATGGAAG
AAGTGTGGCAGAAACAGATGAGATCCCTACAATCCAGTCTCTCCATTGCAAAGAAGAGCCTAGCCATTGA
TGATTCTGAGAGAAATTCTGATGCATCTGTCAATGCAAGTGATGAGAGAGAGTTTAGCTGGGATACAGGA
AGCAACCATAGGGGTCAAGAAAGTAACAGTGCTAGACCAATGAGTGCAGGCTTGAGTGTTATAAGCCGTA
TGGCTGAAGAATTTGAGCAGAGGAGTCAAGTGTTTGGTGATGATGCCAAGTTCTTGGTAGAGGTAAAATC
AGGTCAGGTTGAAGCAAGTTTGAACCCTGACCGAGAGCTCAGAAGGTTGAAGCAGATGTTTGAAGCTTGG
AAGAAGGATTATGGATCGAGACTACGGGAAACAAAGGTGATTTTGAATAAGCTCGGAACTGAAGAAGGTG
CCCTTGACAGGGTGAAAAGGAAGTGGTGGGGAAGGAGGAACAGCACGAGATATACTTAGATTACTTTGTT
TGGTGGTTAGCTCCTGGCTATTTAACATCCGAAAATTATATAGCAGAGAAAAATTGTGATGACTTCTTTT
TGTACAAGCGATGTGGTGATGGCAGAACCTCCTGGTTCTTATCGATGCCTAAATTAAAATGTACATCATT
CTTTTTTATTATCACACAAGTTGAAGAAGCAATATTTTCTTCCCATGGCGGTGTCTATTTCCTGGTTATT
TTTTTTGGGGGATAATTATAGAAACCCTCACTTTCCTTCTATATTTTTATTAGTTATATTTTTTATCCTG
AAAATACATTATTGTTGAACCTACTGCAAGGTCTCTCCCTCACGTTGCACCCACACGCACTTAGAGTGCA
GCGAGCCTCCTTCAATCCCTAACCTGCATGGATATTAAACCCGGCCTGGGAGTTAGACCTGGTTTGATCG
AATCTTGGGTTTTATGGGTCAATTCAGAAAAATTAAAAAAAATTAAAATTTTAATATTTTATATGAAAAG
ATTAAAAAAAATCCATGTAGATATCGATTATATATATTATTAATAATTATCGAATGCTCAACCTAGATTT
CGGGGATGATGATGTGAATTCATAAGCTAGAATTTGATGATATTTATATTTTATTTATTACCTGTTAGCT
AAGATAATTATCTAAAAAAGAAAAATTCATTCTTTCATATCGTTTTGGATTGATATCCATTATGTTTGAA
AGAAACTGCTATCCCCACTCTTGTTCGCAAAGCTTGGTGCGCCAAAATTGACTTTTAAATTTTATGGTAT
ATAAAGCTGTAATATATATATATATATATATACATATATATATTCATTCGATAATATGTATACTACATTC
ATTACCTATTAAATAACATAACTATTTAAAAAACCATATTCATTCATGTCAGGTTCATATCATTTATTCA
TTATCTTCGGTTTTTCTTCTTCTTCTTAATTTTGCATGTGCCTAAGCCTTGAATGACATAACTCAGTTTG
GCTGACTTTCATGGTTGGGAATGTGAGTGTATACTGCCAACCTAGTTTTTAACAGCTAAGTGTACCTCTG
ACCAATGACTTCTTTTTTGCCTTGGAGTGGATCTCAAAGGATGGTATGAAGAGTCAGGTAATTCCTATGT
TGATCATGGATCAAAGAGTGGCTTTGGGTAGGATCTATAAGAAGGAATTCCTACTCTTTTCTAGTGATCT
GACTAATCCCACCATATATTGAAGTCTTCAAATTTCTTTACAAAGAAGAGGGTCTGTTATTGATGAAGAA
CTTAATATGTTACTGTTAGCTAGTCATGATGGATGTTGATTATTGTGTCATGACATGATTCTTATGCCAT
GCATGTATTGATGGAAATGTTGGTGATGAGTAGTATCATTGACATAGAAAATAGTGTATTGATGGAAATA
CGGGTAATGAGTAGTATTATTAGCGTAGAAAATAGTGTCTGTTCTACATAGAAAAACTCTTATACATGTA
GAAAAAATTATGTGCTTGAAATTTTATGTGCGCGGCTTATAAGAATCCACAAAAAAATACAAAGAAAATG
TGTGTCAATACAATATTAGACCATCTTAGTTGGTATCGAATGAGTAGAAAATTCCTGCCATTTGGGCAGC
TGAAAAATTATTATTAAAAAAAAGCTTAGCAACATCACATGTCTTAAATCTAGACTCAATGCCTAAAAAT
CAGTGTATGTTTCTTGTTTTAAAGTGTTTATCTTGAACCTTAGTCACAGTGAACTAGTAGAGTACAATAA
CATGTCTCCAAGTCCTACCTCTCAGAGACAATTGCACTAAAATGTTTGCTGATTGTCTTAGTTGATCTGA
TGTAAACCATTCCCATGATTTGAAGAAACGGCACGGTAAAAGTGTCTTTGTTTGCTTCTGCTTGTAGATC
ATTTCATGCCAAAGAATAGGACGAAAATCCCCTGCTAAACCATTTTCTGGTTTACTTCATGCTCCATTCA
TAAGCCATACAAAAAAGAAGTCGGAATTAAAGGGAAACATTTCGAACATCATCGGGGGTAAGAATCACAA
CATTTTCAAGGGCACGCAGACTTGAAAGGCAAGCCTTGAAATATTATAAGCACAACGGTATGAGCAGATG
CACTATATAAACCTCAAAAGATGCAAAGAGATATGTGTGCTTGCCTGCTTTCTAATTCCTTCACTACTAT
AGTAGCTGCTACCACTGCCAAGTCTATCATGAAGTTCACAAAACAATCCCAATTCATTTGTCTGGCCTTG
CTCTTCGTGTTGGGAGCTTGGCCTTCCAAATCTGTCGCTCGTACCCTCCAGGATGTGTCC
Sequence ID: 71
Sequence Length: 3000
Sequence Type: DNA
Organism: Poplar sp.
ACAATATGAGAAACTGAAAAGGTTCCAATTAAGTACTATGATGCACTCTCTCTAAACACGAGTAAGGGTT
AAGGCAATTTAGAGAAAAGTTAAGACAATCACAGAATGAATTTAGAATAGCATATGGCCTATGACCACTT
CGTTACCACCCTCTAGCAAAAAATTACAGACAATAGTATCACATTTGCATCTTTAAATTACGGCATTCAA
CATACTGCCAACTAACACCTAGCAGCAACACGTCAAATTCTAAGCATGAATTTGACTAATTGACATCAAA
AGGATCAGATTTTACAATGTAAGTGTCAGATACCACTAGCAAAACCAGAAAGCCTATGACTTTGGAAGTT
TGATCTCTTCCTTTCTCTTCAACCTCCTTTGTTCCTTCCAATCACCAACTTTCTCTGAAGCACTGCAATG
AGCTGATTTAGAATCCTTCCAGTCCTTACATGGAGCAGCACAATCACAAACATAGCAATAACACTGCAAT
CAAATCAAATCAGCATAATTAAAAACTAACAAAAACGAAACACAATCCTCATTAATAATTTACCAGTTCA
CAATAGCTCTCATGTGGGGTTTTATCAAATGGGTATTTAGTACAAAGATGCCTTGAGTGTGGATAATCTC
TACAAGCCACCTATACAGCAAAAAAAGAAAAATCCAATAAAGATCAAAACTTTATAACCTATCAACAACA
AAAAAAGCAAAACCCTATATCCTACTTAGCCAAAAATAAAATAAAGAATAAAAAAAGACCTGGCCTTTTT
CAGCAACAACAGAGAGGTCATCGACACCACCACCATCATCAGTGAAGGAGGAGACAGAGAGAGTGGAGAT
ACGATGAAGAGAAGTGTCAATATCAGAAGGGTCGAAATCAAGGATAAAGCAATCCTCAATCTCATCAAAC
TTTCTAATGTTTTCCGTGTTTGTTTTCTTCAAACAAAATATGGGTCTGATGGGAGTCCCATCTGGAGACG
GTGAAGATGTTATCTCAATTACGGCTGATTAATCTTTAACCTCCATGATTGTCTATTTGCCTTCCAGGAC
AATAATCAATACACTGGTAGTCTGGTACAACACCGAGCAAGTTTTAATCCAGAGCACGCAGACTCAAATT
TCAAAAACTAGAATATTGTACGAAAAGAGACCGTTGCTTTTCAAAGAAGGCGCGTATTGCCATAGAAGTT
TTTGTTGGGCTGAGGAAGTATTTGTTGTTGAGATTTAAGGCCTAATCTATTACAAAGTTAAGTCCAAAAC
AAAACAAAATCCTGGCTCGCATGGAACATAATTTTCGACAGAAAATGTTTTCGTGACATTGCAATATTGT
AGCCCATCATTTTATGGGGCAAAATAGCCAAGTTCTTTCCTAATTCTATAGATGGTGAGCCCCTGCGCTT
TGATGGCCGAGAGGCCCGAGACACATAGGATGAATCTATGCACTCGTACGTCGAGAGGACACTATACGCA
GAAATTATTTCCTCCAATGTATAAACTTCATCTGTTCAACATGCAACCCATGCACTATACAATTATTTAA
ATCTCACTTTTACGTAAGAAAAAAGAAGAAGAAAGAATACAACTTTTGATGTATTGATATAACTCCATGA
GACTACATATGCCACCACTCTTCTCTCTCTTTTTAACGATGAGAGATTGGTCAGATCGAGGGTGAAGGGC
TACCCAAACTCTCGTGGGGGCTAAGTTAGAAATGTCATGGGACTTTAGGAATGAATATTTGAAAAATCTT
CAAGGATTGGGGGGAAAGGGCTGGTAGTAACTCTCAAGGGATTAATATGAAAATAAGGCTAACTTTCTAG
ACAAGAAGTGAATTTTTAAGAATCTTGGAGGGTCATGCACGGAGTCATAATTGAGTTATGTGGATTGTCG
TGCAGGGGTACTTATGGGTGAATAAGTTTGTATCGAGTGTGTGGTGACCATCATGCTACCCAAGGTCTAT
GATATCCGCAGAAGCATGCAGGCAGCTTGGCGTGACATAAGCTTTGCTAATTACCTTTTAATCACTACTT
CATCACAAAACCTGTTATTAGAGCTTGAAATGCGCACCTAAGACCATAATTTATTGCATCTCATAATTAA
AGCAAAACCTACCAAATATGATGCGAAGTTGTTCAAGTCCTTCTACAAAGTCAACTTCGTACCTTGGGAC
AACACACCCTCGATAGAAGGTAATAATTAAACCATTATCTCTCTCGATCTTAGAATATTAGACATATATG
CAGGGGGTTGGCGTGCTTCCATTAATCTTGTAAAATAAGTGATTAGAACAGAGCTACATGCTGGGGTGTT
ATACGATATAAATGTAGAATAAGTGACAGAAATCTCCACTTGTCATATGAATTCTTCTAGGTTTTGGATT
AACAAAATATGGAAGCAGCTTGTTTGTTTAATTACCTCAAGCGGTACGTGCGTGGAGCTAGCTAGCTAGC
ATGATCTTAATTATTGAATGTATGTGGTAGGTAGGTTTTCGATTGATTGATAGCAACCTTGTTCTTAAGA
AAATATTGTAATGAGAATCCTTAGGAATTATAATATACTGCCATGTTGTTCTGACAAATAACAACATGGC
TGGCGTTTGATAGAGATGCAAAGAGCGACGACCTTATTTTAAAACATTTAATTACTTTGATGATCACCAT
ATCAAATAGCCTCCCGACCTAACCTTTTTTATACTTATACTTGGTCAAGATGATGCCACGATCGAAGACG
ACGACTACTACTATAATAAGAATAAGAATAGGAATAAGGATGAGGAGGAGGAGGCACTGTGCCATTTTAG
GCAGAGGTTGCTTAAGGGCAATGACAAGAGAGTAGGCACCAACTACAAGGTGGTTGAGTAGTACTTCCCC
ACTTCACCATATATATATACTGTACACCCCTGTCCTAGCGTTCCCATACCCTCCCAAAGCATTTCTTCTC
TTCTCTTACACCTTCTTCAACCCTCTGTTCCCTTAAAAGAATCGATCAATCTTGATCACG
Sequence ID: 72
Sequence Length: 3046
Sequence Type: DNA
Organism: Poplar sp.
TCATATAAGGTAGGCCACCTGTTGCATTCTTGCAGGTTTCGAATAAAATAATTACCGAGGGATATATTTT
TTCTGCTAGAAACCAAAGACTATTGTTAGAGTTGATTAGGAAGGTGATGATTTGAGAGAATATAAGGAAA
TTAATTAGATATGAAAGTCTCCAATTTGAAGTTGTTAACCCTATAGTGAAATTTTCGAGGAGCTTACTCG
TAATAATCATAATTATATTTGTACAAGATACAAGGCATATTGTGGTATTGCACTTAAGAGTAGATATAAT
TTTGTTTGAGAGTGTGGTTGCGATTGCTTTTTAAAATGTTTTTTGTATTAAAATACAATAAAATAATATT
TTTTTATTTTTAAAAAATTATTTTTGAGATCAGCGTATCAAAACGATACAAAATATAAAAAAATTAATTT
TTAACCAAAAAAAAATATAAATTTTAATGAAACGTGCATTCCCAAACCCTTTCTAGACAACCTTACAAAT
TGAATGGATAAAGGTAAATAATGATAATCTTCGCAACAAAAAATATATCAAAAAAGCAATATCAAATATG
AAGTGTTAAGCATTAATAATTCATATGACGTTGGTATTTTATTCTTCATATTTTTTTGCATGGGTTCAGA
AATTTTACATAAATATATCTTTACTTTGAACCGACAATTTAACTTAAAAAGAATTTTCTGTCAAAGAACC
ATCTCAACCTAAAAGTTTAAGCTATTGGGTTGAGATGGTTCTTTGACATGGTATCAGAACCTTGATGAGC
AAGTGATTTCAAGTTCGAATCTCATCATCCCTATTTATTTGATAAAAATTAAGTACAAGGTAATGTGGAC
CCATGCAAGTTTTAAGTTTAAATGACTTTTACTTAAAGGGGTGTGTTAGAGAATAATATAAATCATATCC
TGGAACTTCACATAATAGCTTAAACTATTGGGTTGAGATTGTTCTTTGACATCCTTTTTATTATATTTTT
CAAAGACATATATCCTAATCGATTTAATATATATCTCCCACCGGTAAATATGAACTGCCCATACGAAATT
GAAATCCTCAAAGTCTAGGGTCTTGTTGTTATCTTTTGACTTAGTTTATTCTTATCATATAAGATAGTTC
TTCAATTTATTTTTAAAAAAATTAATTGAAAAGTCATTTGATACAAATCTAATCATATTCTCTCTCTTTT
TCTTACAAAAAAAAATAAAATAGCAGGAAATTGTGTAACTAAAGACCCTATGACCCTATCCTTGCGAAAA
GAGAAGGGAAAAACTTGTGTAGTTGTGGGACCTCGTTTGTCACGTGTTTTGTCGGTGCAAGGAGAGGTTC
AGACATGAGGTAAGCACTTACGCAATTGGCTTAGGCAAAGACTTCGTCACCCACTTCTTGAAGTAGCCAA
TTGCATAACGCCATTGTAAACACAACTCTAATTGCCCAATTTTGTATGAATTATTCAGGAAACTGTAGTA
GATCTAATATTTTCTAGGAAAGATACATCTAATCATTCTTCAATTTTGTAAAATAATATAATACGAATAG
AGTTAATAATATTGTCGTTGATTGTTTTTATATTTAAAATTATATTTGAGTTCGTTTTAAAAATATATTT
AATTTAAAAAATATTAAATTAATATTTTTTTTTATGTTTTATGATAGTTTTAATATGCTATTGTAAAAAA
AACTTATTTTTAAATGAAAATACATTTAAAAAAAAATGTATATTAGATTTTTAAAATTTTTTAAATATGC
AAAAATAATAATAAAAATTTGGATTATGACAATTATATTCGAAGTATTTATTCTTTTGGGGAGGAGAAAT
TCAATTTTTAATACATAATTGAGAACAAGTTTTTTTCAAGTATGAGAGATTGACGATTGACGTAAAATAA
TTATTTGAGGAAATTATGAACCAAATTAACAAGATCAAATCTGAAACAAATTCTATAAAAAAACCTAGAA
GCATTGCATATGAAATATGAAGCTACTATTTTTTGTTTCAATTATTTTTCTAATTAATTTAAAATTTTAA
TTATTTATATCTACCTAACTTATGACTTTTAATTTAATTAATATTTTACTATTTAAAAATTCTAATACTA
GATGAATTTTATTAATTAGATAAGTTTTAATTAGAAATTTATTTTATAAAAAATTTTAGGACTAATATAA
ATAGAAATGTTTAGTATATTTTATGGAGATTAAAATTATTATTTAAATTTAATAAAAATATGAAAGAATG
TTTTTTTAAAGTTTTTCTATAGTTAGATCTCTGACTTTAAAGTTTAATAATTCTAGATTTATCTTTGCAA
TAAATTGTGTCATGAAAGTATAAATGATGGCTCGGCCTGGTACTTGGCAGCATGCCTCATTGCCCCGGTA
CTTTTCAAGCTACCCACAGTTCTGTATTTTTATAGAACCTTTTTGATTATTTACGTCATTTTTTAATTTT
AGGACGTTTGTTTTTTTGTTGTAAAAATATTTTTTTTATTTTAATTTAATATTTTTTTAGTATTTTTAAA
TTATTTTAATATGCTATATAAAAAATAATATTAAAAAAATAAAATAATATTATTTTAATTTAATTTTAAC
TACAGAAATATTATTTAGTTACATTATAAATATTTGTTTAAATTGGTGGCCTAGTCTTTTCTTAGTTGTA
CGTCGGTTATAAAAACTCTCACAGGTGGCCACAGTAGGAGGTCCCATTCCAGCTCATTAGTCTGTTTGGC
TCTAAGTTAAATCTGCTTTTGATTAAATTTCAATTTTTTTTTTTTGCTAAAATTAAGTGCGGTTTATACT
TTTTAGATTGTTTTGATATACTGATGTCAAAAATAATTTTTAAAAAATAAAAAAACATCATTGATATATA
TTTCAGCACGAAAAGCTATTTGAAAAGCAACCATTACCACACTGCGAAACACTCTCTAAAAAGATTTTCC
ACCCATTTAATTCAAAATTTTTCCATTATAAATAAAGTGAATGGTGGGAGTCCAATATGCGTCCAGTTTC
TATCTAAAAATCAACCAACCTCCTCCAAGAACGAAC
Sequence ID: 73
Sequence Length: 3000
Sequence Type: DNA
Organism: Poplar sp.
TTAAAATTAATATCATTTTCAAATTCTTTAGCTGGAGATGCTTTTACTTGGTTTATAAATTTGCCGCCAA
ATTCAGTCCAGAGTTGGAATGATATGTAAACAATAATCCAAGCTCGCATCTCTATAACAGAATCATAATT
TGTTTTGGCTAATTTGGTGAAATTGAAGCAATATCCTGAACAAATTGTTAAAAGATATTTAGATAGGTTC
ATGATGCGATCTTGGCTATGACATCAAAGAATTCATTAGGAGTGTCTATATAGTCTATTTTAAGGTTTAA
AACGAAGTGGATCCAAGAGAAGTTTGATGGGTTAAGTCAAGAGGTTTAAGTTCATGATATAATTTTTATT
TGGGCTAGAAATTATATTAGCAATACTTTCGTTTTTTTTATCACATTGAAAAAAAATAGATCTAATATTT
TTTTAACATTAAAATTAATTGTATCGGACTCGCAGCTTAGCTCATGCTAATTATGTAACAAAAGTAAATG
TCTTCGTCATCATTTGTAGTGGGTTAATTAAACTACTACGAGGGTTATTACATTAGTTTACGAAATTTGG
CATCATTTCGTAACCGATCATACCGATCACGTCAAGAGAAGTCTGACAGGTGGACAAAGCTTTTTCTATG
ATTGTACTAAATATTCTAATTATTCCATTTGTCAAGCTTTTTTTTCTAAACAGACTGCGGAAGAGTTGCG
TGTTGTGTCATATAACCTATTATTCTTTTGACTATTTTCATGATAGCTCTATTGATTGAAAAATTAAAGT
TCAAATTAAATGTTTTTAACTTGATTTTATACTAAAATATTTAAAAAATAATCTAGAAATGTTAATATAG
TCATTTACAACTACAAATGACATCTTCCTTTATTTCTTTGTTGTAGTTTACAAGAGTATGTTTGTTTTTG
TATTATTTTTTTATAATTTTTTTTATTTTAAATAAAATTTTTAATAATTTTATATTATTTTAATGTAATG
ATATTAGAAAGAATTTTTTTTTAAATTTTTTTAATATAATTTTAAATAAAAATCACTTTATAAACAACTA
ATATCACAATTTTAAAAAATAACTTATTGATTTATGCGTCAATCAATTGATTTAAGATGAAATGATTGAT
TGTTTGATTTTTTTATGTGGTTTTTTATATTTTAAAATGGGTTTTGATTATTTTTTTTGTATAATAGCTG
TTGTATTTTAAACAAATAAAATATAAATTTGGCAATTAAAATAAATAAAATTAAAAATAAATCATTCAAA
AAAATAAATGAAACGTTAAACACAAAATCTGCGGTTTTGTTTTAGTAATGACATAATTATATTTTTCTCT
ATTAATAGATTGTTGTAGCAATAAATACCACATTTATAAAAAAACAATACAGAGAAGAGATTATTAATTC
GTATTAATAAAATTAAAGATGAGGGAATCCTTGAGATAACTAATGGAGGGGTTACAATATTGCAAACAAA
GTTTTAGGGACCACAGTAAGAAAATATAAAGTATTGGAGTTTAGTTTTTATTGAATAATTTTTACATTGT
GGATTTAATTTTGCAAACTGACATGCTATGACCCCTTTTTGTCTGGTGAAACATGTTTCTTTTAAGGGTG
TGTTAGTTTTCTGGAAAATGACAGGTTACTTTTTAAATTTTCCCGTGTTTGTTTGTTATTATAAAAGTTG
ATCAACGAAAAATACTTTCCAGTCAAAGAAAAATTTGGCTTGGTTTCCAGAAAAGTGTTTTCCTTTTATT
TTGGGCGGAAAACAATTTCTCGAAGTTGTGAAAAATTTAGAAATGTCATATTATTTGCTGATTATATCAA
ATTTGGTCCTCAAACTTTTGATTGCTATATATATTTTGTTTTGAATATTTGTTTTTCAATTTCATCCCTT
AGAATTTAATTTTTATATTAACTTTGATCCTCATTTTTATAATTGTTATTTGCTTTTCCCTTATTATTTT
TTAATTGAAATTTTTTATCTATCAAATATGGTCCTCATTCTTTTGATTGTTACTTATTTTATTTGAAATA
ATTTATGAAATGGTAATTATTATTATTTTAATTTCTTCATCTTTTAATTTTTTTTTATTTTTTAGATTTG
ATCTCTATTATTTTCATTATTATTTATTTTATTTGAGATAATTTATGAAATTATATATTTTTTTCAATTT
CATTCTCATTCAACTTTTTAATTTGTAAGATTTGTTCCTCATTATTTTAATAAACTTGAAAAAAATTAAA
CATTAATAAGTTATTTTCCAGTTCATTTTCCATTACATAACCAAACACTGGAAAGTGTTTCCAACTTATT
TTTCATTACACTACCAAACATTGAAAAATAATTCACTTTTTCGGAATTCACTTTCCAAATAGAAACTACT
TTTCAGCAAAAAAAAAAAAAAAAGGGCTAAAATGCAACTAGATTCTTGATATCATTTAAAAGAAAATACA
AAAAAGAGAAACAAAAGCATTACAAGTGTGTTTTTTTTAGAAAATAAAAATCAAACATCTAAACAACATA
ATTTCTTCTAAAACACTAAAACTATTACATGTCGATCGCGTCAGACAACCTATTACTCTTTTGATTATTT
TCATGTTAGCTCTAATGAGTTCAAGTTAAATGTTTTTATATTTAAATATCTAAAAAAATATTATATAAAT
GTTAAAAAACTCATTTACAACTACAACACAATCTCTAATGAATGTATTAGTTAAACATTGTTCTTACTCT
AATGACTGTATTAGTTAAACATTGTTCTTACGACATTTTCTTTTATTTTTTTGTTATAGTTTACAAGAAT
AGTTTGTTTTTGTGTTATTTTTTTTTCAGTAATCTCGATTAAATCAGTTTTTCAGTTTTTTTTTACACCT
CTAATAAAAAGATTACTTCATTGTCAACTATTTCATCCAATCTTCCATTATAAATACTTTTGAAGCCGAA
TATTCATAAACCCATCCGGTTTCTTCTATCTAAAATTGATCGACATCCTCAAAAAACATT
Sequence ID: 74
Sequence Length: 3000
Sequence Type: DNA
Organism: Poplar sp.
TTATTTAGAAATATATTAAAATAATATTTTTTATTTTTAAAAAATTATTTTTAATATCAATATATTAAAA
AAAATATGTAAAAAAATTAAATATATTTTTTTAAAAAAATTTCATATAACGCGTGAGTGGTACGTTCCAT
TATGGTTGATAAAAAGTAAGGGTGATCATTTTCAGTTCGATTCAGTTTTTACCTATAAAAACAACCAAAC
CGAAAAAAAAAATTTATATAAAAAAAAACTAAAACCGAACTGAACCTGGTTCCAACCGACCGGTTTCGGT
TCGGTTCGGTTTCTATGTAAAAAACCGAAAAAACCTATATTGTTTTTAACATAAAAAAGCCACAAAATCA
ATCCCATCTTCATCACCAGAAGCCCCCACGACAGTGTTGAATCCATACCGGCCTTTTCTCAAGCAAATAG
CCTCCTACAGAACCACAAAAACACATCACGCTCTCTTACAATAATCAATCACTCTTCACTATCACAACCC
CAAAAACCTACATCACCGAGAACCATACTCTCAAATACAAACCATCACCGCCACAATAAACACTGTGCAG
TGCCCACTGCCACTTTCTTTATCCATTTTCCTTTCATAAAAAAAGAAAACTCAGCAGCTGCCAACAACCC
AAACCATAAAATCCACTAATCACCTGCAAATTAAAGAAATCTCACTGCTGCTGAGGTTTATGGGTGTTAT
TGGGATGATAGATCTGGAGATGATTGAAGAATGAAGATTGGAAGGTTAGGTGTGGCGTGTGGGAGCTTGG
GTTGGGGTGGTTAAACTTTGAAGAACGAAGAATTAATGTTGTGAAGAATTAATTGTGCTGTGTGCTATGG
AGAATTAATTGTGAGACGTGAGTGGACTTGTGAGCATAGTTATTAAACCCGGCCCGGGGTTGACCCGGTC
AAGGGGCCAGGTCTCGGGTTTCATGGGTCAACCCATGTCAACTCGGGTCAACCTGGATCAACCCGAAAAA
TTAAAAAAAATTAAAGTTTTAATATTTTATATGAAAAAATTAAGAAAAAATCCATGTAAATATAGACTAT
ATAAATTGTAAATAATGAAGTTTAAAAGAATATTTTAAAAAGTTTTTTATCCCATAATAAAAAGATATTA
TGTTAAGCTCTAAAAAGTTTTTTATCCCACATTGAAAAAAAAATCTTTTTTCTTGGGAACATAAAGTATA
TATACTAATAGGTTTCAAATCCCACATTGAAAAAAGATAGTTTTTTTTTTGGGAACATAGAGTATATATA
CTAATAGGTTTCAAATCACACATTGAAAAAACATAGTTTTTTTTCTTGAGAACATAGAGTATATATACTA
ATGGGTTTCAAATCTCATATTAAAAAGATATTATGTTATCCTTTTAAGTTTAAGTATTTAAACCAAAAGG
TTTTTTTTTTATCTCACATTGAAAAAACATAACTTTTTTCTTGGGAACATAGAGTATATATACTAATGGG
TTTCAAATCCCACATTTTAAAAAAAAAAAACCTGGGTCTCACCCGGGTCACGGGTTGACCTGTCAGGTCG
ACCGGGTTTGGCCGGGTCGTTGCACCGGGCAGTCTTTTGATAAAACTGGACCGGTCCAGCCCCCAGGTCG
ATCGGGTCCCGAGCCAACCCACCGGGCCGAGTTTAATAACTATGCTTGTGAGAGCTTGGGTGGGGGTAGT
TAAAGAACGAATAATTAATTGGGTGTAGAGTCTGTAGACTTTTGAGTGTTGTGGGAGGCTGTGCCGCTGT
GAGGATTGAAATGGTGTGGCTCGTGTGGGTTGTGTGGTGTGTGTTTGTGATTATGAAGATTGAGGAGTAT
TTATAGATTTTTTTTTTAATTTAAACCGGTTCGGTTCGGTTCAGTTTGGGTTGCTCGTTTAAGTATTATA
AAACCAAAACTGAATCGAACCAGAAGTTTTTCTAAATATTCTAATCGGTTTTTTTTATGGTTCAGTTTTT
TCGGTTAATTGTTTTTCACTTTTTTCAGTTTAATCGATTTTTTTGCTTACCCCTAATAAAAAGATTACTC
CATTGTCAACTATTTCTTCATCTTTTAATTTTTTAATTTTTTAGATTTGATCTCTATTATTTTGATTATT
ATTTATTTTATTTGAGATAATTTATGAATTTTTTTTTTCAATTTCATTCTCATTCAACTTTTTAATTTGT
AAGATTTGTTCCTTATTATTTTAATAAACTTGAAAAAAATTATACATTAATAAGTTATTTTTCAGTTTAT
TTTCCATGACATAACCAAACACTGGAAATTGTTTCCAACTTATTTTTCATTACACTACCAAACATTAAAA
AATAATTCACTTTCTCGAAATTCACTTTCCAAATAGAAACTACTTTTCAGCAAAAAAAAAAAGAAAAAAA
AAGAAGCTAAAATGCAACTAGATTCTTGATATCATTTAAAAGAAAATACAAAAAAGAGAAACCAAAGCAT
TACAAGTGCGTTTTTTTAGAAAAATAAAAATCAAACATCTAAACAACATAATTTCTTCTAAAACATTAAA
ACTATTACGTGTTGTGTCAGACAACTTATTATTCTTTTGATTATTTTCATGTTAGCTCTAATGAGTTCAA
GTTAAATGTTTTTATACTAAAATATCTAAAAATTATTATAGAAATGTTAAAAAACTCATTTACAACTACG
ACACAATCTTTAATGACTGTATTAGTTAAACATTGTTCTTACGACATTTTTTTTTATTTCTTTGTTGTAG
TTTACAAGATGTTTTTGCGTTTTTTTTTTTCAGTATCCTCGATTAAATCGGTTTTTCAGTTTTTTTTTTT
CACCTACAAAAAGATTACTCCATTGTCAACTATTTCATCCAATCTTCCATTATAAATACTTTTGAAGCTG
AATATTCATAAACCCATCCGGTTTCTTCTATCTAAAATTGATCGACATCCTCAAAAAACATTATGGCATC
TTCTATAGAAATCACGTCAGTCAAGTTGATGAAGCCAAAGATGAAAACTTTGGATATGCC
Sequence ID: 75
Sequence Length: 3075
Sequence Type: DNA
Organism: Poplar sp.
TTTTATTACTTTATAAAGTTTAATAACATAAATTTTCTATAAATTTATTTTTTAAACTACATGAGAAAAA
AATAAACTATAAAAAAAGTCAAGTTGAATTAAAAAAAAAGAAGACAATCCACCAAACTCGTAAATCAGGC
AACCTGAGTTACCTTGGTAAACTTGTAAACTACGCTAACCTCATATAAAGACAAAATAAAAAGAAGCAAA
TTACAAAGTTAAATTCTCTAACAATCTCAATTTTAAAAGATGAAATCAATAGAAACATATTTGAGAAAAA
GTTATCGTAATAAAAATTCAAAAACAAAAAACAAAACATTGTGGACTATAATATAATCAAGTTCGATTAG
GAAAAAAAAAAATACCCTACTAAACTCACGAATCGGGACAACCAGAGTTACCTTGTCAAACTCACAAATC
AGGTCATTGACTCCACAAAGTTTAATAACCTAGTTTTTTAAAATTATTTTTTATTTAACTAAACTTTTTA
AAAAATAGACCATACTCAATGCTAGATATTTCTTTGATACCAACTCGATGCTGAAATATTTTTTTGATAC
ATCAATAATTATATAAAAATATAAATTAAAATAAATTATCAACTCTAAATCCTCATAAATACATCGTATA
ATCATAAAAGGATTGAATTAAAAATAAAATCAATAACTTAATGAAAAAAAAGCATCAAAATGCAATCCAA
AATGTTAATGGGTGTTGAAAACAATATTTTATCTACATCTTTAACTTTATACTTAATAATAATTAATATT
TAATTTAATAAAATATTAATATTTTGTTCATAATTTAAAATGACCAGTCCATTTTTTTGTTCCAATAAAA
CAAAATACAATGCATCTTATTATCAATAAGAGTTGGTTTAGAAGATCGTGCAGCGTGGTTAAAACTATTT
ATATATATAATTATAGTTTTAAAATTATTTGATTAATCAATATATATTATAGTGTTACTTATTTGTTTTT
AAAATTTTAATTGAGAAAAAATTATAAATTATAATTGTTTTTAAACATGTTTTTTGAACCTTAAATTGCA
AACCGCAAAAGCTATCATATCAAACACACACCATGTATTTGATAATATAATCTATTGTATTTTTTAAATA
AAAATATATCAAATAATTTTTGTATTTTTAACAAAAAAATATTAAAACCATCAATAATCATATAAAAAAA
CAGTTCGAGAATAACTTTTAAAAAACAAGTTAAAAATTTATCATTTTCATTGTTTTGAAGTAGAAATAAA
TGCGAGGGGCTCGTAGTGGTTGTGATTCTTGTGAAAGCCAGTAAGTACCAAGGTCACTGTTTCCTAGGTT
AGAAAATAAGGGCACTCTCTCTTGTCGAGTACACTAAATTTATATCAGATTTTGAGATGGTTGTCATCAA
AGCGTGTCTGAAAAGGAGGAGCAGGAGACAGGTGGGAAACTTAAATATGGATTTGATGTCTGATTATTCC
TTCTACCTAAGATATTGATGGTTTTTTTTTTTTTTTTTAATTTTAGTTTAGAATATAATTTATAGTTTTC
TTTTAATTTATATTTAAAATTTAAAAATCATTGTACACCATAAAATTATGAAACCATTATTCACATAATA
CTCATTCTACATAATAAAATTTAAAATTTAAAAATTATGATTCACTATTTTTTTTAAATCGAGTTTTAAA
TTAAATTATGTGATAGTTATCCTGATGTGATCTTATTTATTAGATAAGTCAGAAAAAACATTAACTCAAT
AATGAAAAAAATAATTAAATAAAAATCTAAAATTAAATGATAAAATTGAAATAAAAAATCTAAAAATAGA
GAGTAAAAAAATCTAAAGAAAGTAAGAAACTCATATTAAATGATAAAATTAAAAAAAATAAAAAATAAGC
CAAAAAACTTTAATTACTTTAATTACATGATAAAAAATACATAAATTGATTTACTATCAATATAATACTG
AAAAATAAAGTTAAATAAAATCTTATATTAGAAAATAAAATAATAAAATAAATTTTATATTAAAAAATAA
AATTAAAAAAAATCCCCAATAACTAACTCTCAGAAAAGGCCATAACATAAACTATTTTTCTTTGCTTTTT
ACAAATGTCCAACCCAATACTCTATAGTTGTATTGATAATAAAAATAAAAATAACTAGATTTTTTATTAT
TTTATTTTTATCATCAAACTGAAATCATTAAATTAAAAATTAAAAATTAATGATGAGATTGGAAACTGTT
TCCAAATAATGCAAATCATTTAATTACAATAATGTTTCCATTTTTATCATATTTTAAACTAGACTATCAT
TCTATTTAAATTTAATAAAATTTAATTATAAACTAACTAGAAAAAACTATTTAGTCCATTTTATAAAACT
AGAAAAAATAATTTATAATTTATTCAATTTAATATATATAACAATCATATTTTTAAAAGAACATGTAAAA
ATATGGTGAAATTATTTTAAACCATAGATTTAAAACCTTATTTAGATAAACTTGTAAAAGAATTTAAAAA
ACTTTAAGTTTTTTTGAAATCAAAACAACATTTGTTTTAAATAAAAGAAATTCACAAAAAAGTCAAATGA
TTAGGCTCATAATGACCCGGTCAATCAGGTTTTTGAATGAGTTTTAAAACGTAAAAAATTCAATTGGAAT
AAAAAAAGGAGTAATAATTCAAATTTAAAAACAAAACATAACATAATCAATTAATTCTTAAAATAAAAAA
GGACTGCTAACTAGACACACGTCTCAATCCTCTTTAAGAAAAAGTAACAAGAACCGTAAAAAGCGAAAAT
GTGGCTTTCTGGACATGTATGATTGTTGTAGTTGATTTGTTTGTTTTAAAAAATTAAAATTTTTTTATTT
TAAATTATTTTAATGTAATAATATTAATGATAATTTTTAAAAGATTAAAAAAATTATTATTTTATTATAT
TAACGAATAAAAAATATTTTAAAAAACAACCGAAATTACTTTCTCGAATAACCTTTTATATATATACACA
CACGATCACTGGGTTATTGTTCTGCACTGAGTTTGAACTCATCAAGGAAGTGAAAAGCAGAGAGA
Sequence ID: 76
Sequence Length: 3000
Sequence Type: DNA
Organism: Poplar sp.
CATTTTCATCCATGTCTTCCACGAGAGCATGATGTTTGATCTAAGTGGTTAAACAAAAAGAATTCTTTCC
CAGTTTAATTTCTTTAATTTAAATCCTAAAAATCAACATTTAGAAAAACTGGTTTCTCTTGAACGATACC
ACTCCCATCTGTCTAAAAGATAATAGATTCTCACAGGGGTGATGATGATTTAGGGCAAGTAAATTGCCAG
CATAAAAATGATAGACTCCAGGAAGAAATTCCTTTTCCTTGTAGCCGTAAGCTTAAGAATGAATTCCGGG
GATGATCATGATCCCGTCACCTTGCTTTCATGCACAGGAGGAGATTCCGTGACTAGATGGTAACTGGGGG
CCATCAATTTGATTGGACTTGGAGATTTTTGTAGGGCTATAAGATTGTAAATATTTCTCTCGCATATCAT
ATCTTGTTTCTCGTAAATCTTTTTCTTTTTAAGAGTCATTCTGGAAATTATACGAACAGTTCATCGTCTA
CCTTTCGCAGTTAAAACGTTTGGAGTGCCCTTGAAGGTTTAATCATGGTCATGGGCATTTTCTAGTTGGG
CTAACTAAATGGAGGGGCAGGCTACTTCAGAGCAGAGCTACACTCGAGCGCAAAGGAGAAGGGGTAATCA
TTGAATCCAACTAATGGGATTCTATCAATAAGATAAATCATTTAAAGTGAGGTTGGAGTAGCAGTTATAG
GTTGCATATATTAACCTACGCCAATCATTTTATTTTGTAAAAACAATATTAAAATATATTTTTTTTCAAT
GTTAATAAATTTTGACAATCTTACATTGATCAATTTTATATATATATATTTTTTATAAAACCCAATATTT
ATTAGATTAATCTGCTGGGTTGCTCGGGTTTTAAAGATGATTAAAAAAAAAGAAGGGTTAGACTAAGACT
TTAATTGCATCGAATTAATTAGACTCGAACGTTTTCGGGGCTATTACTATTTTTATTTCTTTTATTACGC
ATGTAATTTTTCATTTTTCCTGTTGGAATTTACCTTTAATCAACTGCAAGGCTTAAAAGAATTTAATCTT
GCCCGTTCACTTCTAGGCTGGGCCGAGTGGCTGGGCAGGCTACCAATTTATAGACACCCCAAAAGCCCAC
TTCAGCGCACTCCTCCCACAAGTGTTCCCACAAAATGGGCCTGGCTAATATGGATCCAACTCCATGGGAT
CTTCTTACCAAAGGAAGATGATTTCCGCATACAATTGCACAGTAATGTTAATCTCAAGGAATATTCCTGT
GGAACTCTCTTTTTGAAAAACCCCAGGGAAGATAAATTATTAGTATGTTAGAAGGAAATAAATATATTTT
TCTGATATACATGATAATTATCTCATATATTGAAAAATGGATCCTGCTGAGAAATTCCATAAATTCAATA
GTAATATTAATTTAATTCACTTTTTTCAAAGAAAATCTCGTACATTAAATGAGGATCTCATGCATTGAGC
AAGTGAATCTGATACGTAAAGTTAGGATATCATATATTTAATAAGTGGATTCAGTGTCAGAGCCCCCAGC
AGCCACTGCTTAGCATTACCAGAACTATCAATTCGAGTAAAAAGCTAATTTTGTTCAATATTTTTTCTCA
AAATTATGTTCCAAATCAACGCTCCAGTCTAATGTTAAGTCCCATAAATCTGACGGTTGAATGTTTTTGT
AAGAGATTTCGACAAAACCTTTAAATATAATTACCTCGGTTCCTGACTAGTTCTTACAAGGAAAGATAAA
AACAAAAAGGAAAAAAGAATTGAAAAGAAAAGTAGATGAAACAGATAGGATGAGAGTTTTCTTCCCTTCT
TTCCACAACTGTCGCCTGCTGACATTTATCTCACAGGTATCACTGCCTCCCCGTTCAAGGCAAGAAATCT
AAGAGAGGAGCGTTGCTTGGGTCAAACATTATTCTGAAACCAAACACACGCGCATGAAATCGAGCATAGA
ACGTTCGTCAGGTCTATCACTTTTCTTCGGTTTGCTCAACAGATTATGCTGATTCGGTGTCGAATCCTCG
GGGGCCAAAAATGCCATAGAACTTCATCAGATGCAAGATCAATTAAACAAGCAACTAATGGAAAGAAGAG
GCAAAATAAAATGATGATCTCTTGAAATGGGAATTGGGAATATAGAATTCGGAAACAATCCCAGTATTTG
ACCTTTTCATTTTTAATAATATATTGGAGTACTTGTGTTTGGTAAATGGTATGTGCATCAGGAGAAGTTT
TTATTCCTTCCTGTAATCTGATTTCTGTTCCACTTCCTCTAATGGGAAGAGTGTATCCCATCCAGTTTTG
TTTGATAGGTGACGAGGACTGTGCACTTGTGCTAAAAAAAGCACAAATCATAAATGGAGCCAAGTAGTGC
AAACCGTGTTCTCAAAAAATTTTAAATTTTTTTTTGTTAAAATTTAATATGGTTTATATGTTTTGAATCG
TTTTGATGTGTTGATGTCAAAAATAATTTTTAAAAAATGAAAAAACATCATTGACATGCATTTCGATACG
AAAAGTTATTTGAAAAGCAACCGCTACCACACTGTCAAACACTCTCTACTGGTGTCGATAGCAATTTCAC
ATTTTATTTTTGGGTTTTAAAAGTATTTTTAAAAGAATTGATTTTTTTTTTATTTTGTATTAATATTTTT
TTGTGTTTTTAGATTATTTTAATGTGTTGATATTAAAAATATATTTTTTTAAAATATTATTTTAATACAT
TTTCAAATAAATTTTTTTAAAAATATTAATTACTACCACACTTCCATCCAGCACTACTGCTAAGTATGGA
AAGTCAAGTGAGTCTCCAAATGTGGCCATGGAATTCTACCACGTACCTTGAAGCTGTTACCTTCTCATAC
AAGCAATTTTCCTAACAAGATACCTTTTATAGATCCAGTAATCAACCTATCAGTACTATAAATGCACCCA
CTCACTAGGCAATGCATGTTGCCCCAAAGTCACATTTGTGCCAAACACCAACACACACAC
Sequence ID: 77
Sequence Length: 3092
Sequence Type: DNA
Organism: Poplar sp.
GTAATGGAGTTCGGAGTTTTATCAATTACAAACTATTTAATCCGAGAAATATTAATGGAGGTGGTATTAG
ATGTTCATGTAAGAGATGTAAATATAAAAAGTTTCTCGATACAGATGTTGTAACAATGCATCTTCTACAA
AAAGGGTTCATGAAGAGATACATGTGTTAGTTTACACATAGAGAACCATATGTTCCTCACGAGACCATAG
TAGAAATAATGGTTAGGTCAACTTCGAGTTCTAGCAACGTGCATGGAGTTGTAGATGATAATAGTAATCC
TTATAGGAATATGATTACACGGCTGCAATGTGAATGAGATGCGTTATGCTAGTCAATATTCAATCGTGGA
TGAAGAACCTAATCCAGATGCGACCAAGCTTTTTGATCTTTTGGAAGACTCCAATGAACCATTATAGGAT
GGGTGTATAAATTATAGTAAATTATCGATCCTTGCACAAGTATTCATCATCAAGTCAGATCATGGGTTGA
GTGAGACTGATTATTGAAAAAATTATTGAATGGGCGAGAAGCATTTTACCTAAAGGAAATAGGTTAAAAA
ATAACTTTTATGCTGCTAAATCCATAATGAAACCCCCCGGCCTAGGATACAGAAAATTGATATGTGTTCA
AACTTCTGCATGTTGTACTACCTTTAAAATACAAAGTTGACCGAATGTAGAACATGTGAGCATGCTCGTT
ATAAATCCAAAATTAGCAGGGAAATAACTCTTTTCGCACATAGAAAACTTATATACTTCTCAATCACACT
TAGACTTCAAATGATATTCATATCACCAAAAACTGTTGAGCACATGACATGACATCATTCACATGATGTG
GTGGATGGAGTGATAGTGCACTCTTCTAATGGTGAGGCCTGGAAACATTTTAATAGAGTGCATCCTTAAT
TTTTAATAGAATCAAGGAACGTGTGTCTTGGGTTATGTACAGACAGATTCAATCAATTCAGGTTATTTGT
TGCTCCTTATTCTTGTTGGTCGGTGATACTCATGATTTATAACTTGTCACCGAGGATGTGTATGAGGTTG
AAGTTCATGTTTTTATCTATGGTCATACTCAGTCCTAAAAGTTCGGGTCGAAATATAAATGGTTGTCTTT
GATTGTTGATCGATGAGTTGAAGTAGTTTTGGTTACTCGAGGCTTTGACTTATGATGTATCGAGGAAACA
TAATTTTGTTGCCACGCAAATTGGTGGCTCCGTTTGTTTCACATGCGAAGTGGATGGTAAGATTATTTTT
AATAACATCTATTTTTTATTTATTGTTTTTTTATAAATATATTTAACTTACAACAATTTTCTCTTGCTGG
CTACAGTTCTTGGACGTGAGTTAAGCCTGATGAAGCTTTTTATGAAAACGCGCGTGCGAAGTGATGACCT
CTAAAACCAGGTGCAGTAGCTCGTGGATAGTCAAGCTCAACATTTTGCGGTATGTTGGTTTTCAACCATT
TTTTAAGTTATTATTTTTTTGAATTTGCTAACTGTTTTCATGATACATATAACACCCAATTAAAGGAGAG
ATATGAGGACAATTCTTCAACCTGTCCGGATCTCGATCTGAATTTGTGATTGAAGGCAGGTTCGTCCGAT
GGACCCGATAGAAATCAGGTGTACGGTCTCTTTAACACTACGGCCAAGGACTTGCGGACAGCCTGCAATG
TTTCAGTTGTTGGATGCTAGTAATCGATTTGGAGCACTCAAACTCCAGAGTTCAAGGTGATTTTAGACCA
ACAAGTTCAAACTTGAACGACCTAACTTGTTTTTTATAATGAGCAACTCAATGTTGAGATGGCCGAACTT
AGCTGATTTATAATGAAGATGAGATCACAGATGGGTGGTATATGTGCCCTCTTTTATTGGGCTCACGGTC
CTTGTGAAGACCCTCCTCATCCTCCAGCGCCTCTATTCTAAATTAATTGTATTTGAACTTATAAATGTTT
AAATTTATAATAAATATTTTATTGTTATATTAATTTATTTTATTTTAATTATTATGTACTTATTTTTAAT
ATATATATATATTATTGACGGTGTTACCGAAGGATTAAGTTTGTTGGTATATTCTAGAGAGTTGAAAAAT
AATTACTGCAAATGTCACTCCCGCTATTTAATCACCGACAGAATTGCATATGGTCTTTCTGTTGGTTATA
TGTTAGATTCACCAATGAAAATACCGACGATGAAAAAAAATCATCGATGGAATTATATACAGTTTTTTTG
TTGGTAATATATTATATTCATCGATGAAAATATCAACAAAATGAAGCAGGTAAAATGAAAATATCAACAA
AATAAAAAAAAATTTAACGGGCTTTTGTGGTCCGTAAATCCATCAATAAACGTATTACTAATAAACTCAT
CGACAAACCACAAATGATCAATGAGAGGTTTTTTTATAGAACATTTTTATCCATTAATGTCGATATACTA
AAAGTATAAATAATGACAGAAAATTTGGTCGGTAGTGGTTATTTTGACCTCAACATGTGAGCTGGTTCAC
TATATATATAGGTGAAATTGTTCTGTCTTGTTTTCGTTTCATCTCCGTCCAGATTTTGTATGAGAGCTTA
TATTAAATCATAGTTGCTATTTTGTAAGAAAATCATACATGTTTTTTGTGAATTTGGCAATTAGAACTAT
GCTGATTCAAATATATTCGATAGGAGGTATATTTGGATGGCCTCCATGTGCCTGATCCAAAAATATCGAA
AAAAAGAATAGGAAAGTCATTAATTAATGGGCCTACCTTCTCTCCTTATTTATAGAAAGTCTACACTTGT
TTTCAACCTTTCACAAGAGAAAATATAAACTTATTTGATTTCAGCATTGAATAAGTAAACAAATTCTTTT
ACAAATTGGAGACGAAACCTTCCAAGATAATTTAAGATAATTAAATAAATAAAAAGTGGCCAACTTGTGA
AATGTTCGAGACAGGCCACGAAAAAAATCTATATAAAGCCCATGATATCCCTCTCAACTTAGCAGCATCG
GAAAGTTGCGAGGAGTAAAGTCTTGTGGATTACACAAAATACAGACTGAAAACATCCATAGGCACCAACA
CATAAACCATCC
Sequence ID: 78
Sequence Length: 3042
Sequence Type: DNA
Organism: Poplar sp.
TAAATGAATTGACTTTTAAAAGTAACATTTCATTGTTCCTTTCTAGGAATTTGAATATATTCCCACGCTT
GATGTTAAATATGTTTAAAAATATTTTTAGTTTGAAAAAAATATTAAATTAATATTTTTTTTAAAATATT
TTTTATTATTTTGACTTTTGGGTCTGGTGGTCAAGCCAAACCTAATAGCCTTAGGCCTGATAGTCATGAT
ACAACCACGACAGTTGGGTCTAGTAGCCAAGCCAGACTCAATAGCCCTATGTCTGGTGTCCATGTCACAC
CCAAGACAGTTGAGTCTGGCGGCCATGCCACACCCACGACAGTTGGGTCTATCGGCCAAGTCAGATCTAA
TAGCATTGAGTCTGGCATGACTGTCAGACCCAACACCTTTTGGCATAAAAGACAACAAAAACAATGCATT
CATGATGTTACAGTGTTGTCCACAGTACAAACATTTTATATCTATAATGTGGTCCATAAGGTATTGAGTC
TATATCCACAGTACCTGTATAGTGTTCTGGATCTGGAGGCCACGTCAGACCCAAGACGGTTGGGTCTAAT
GACTAAGTCGGATCCAAGTCTCTTGGGTCTGGCGGCCGAGCCAGACCTAACAACTTTTGGCATAAAAATA
GCAAAGACAACGCCCTCACGGTGCTACAGTGTCGTCCACAGTGCAAATACTTTATATCTACAGTGTAGCC
CACAATGCAATGAGTCTATGTTCATAGTACCTGCACAATGTTTTTCCCATGCATTTTAGAGTATAGTATA
ATTTGTCATATTAGTTGGTGTTTATGTGACAAAATATATCTGATAAATCTAAATTTAACGAGCCTAATGT
TGGGCATGCTGCCTAGCCAAAAGGAACGGACCAAGGTTGTTACGGGGCCCATACTAGATGTGACACGCCC
AACTAGGGTGTTGGGTGTCAGGCGAGCACCCTCATGGACTCGGGCCCAGCTACCACGGGGTTAGATAGCG
TGTTTGAGCCTATTTTTCACTCCTATCAGGTTCAAAAAACTTGTTTGAGCCCAACACTTCCTTGGAAATT
TTTCCAAGACCCAATGCGAGTCCATTGGTAATCTTTCGGGTCCCTCATGCGAGTCCTCCCAGTATTTTAT
TTTGATCCGATACATGTTAGTTTATATGGGTGAAATTACTCAGCAGTCCCTCTTCTCCATAAAAGGATAG
GATTCATGATCTATAAATACCATATGAATCCACCAAGTTCAAGGTTCACAATCTCTTCATTCTCAATATT
TTTGACACATATATTTTCGGAACCTATCTCTATCAAATATTATTGAGCTTTCTCTCTCTATAAAGTTACT
GACGTAAGCATCTAAAAGTTCCTGCGTCCATTAAAGGAGACATTTCACAGGTATTTGGCTTCCCATATCA
ATCTACAAGCCACCTTGCCTTTTTACATCAAACGGATAAGATTGCAAGAACTAAGTGATTAATCAATTAT
CTAACACATTAGACTTATTACTAAACTACTTGGATTTTTAAGATATTATCAATATCTCTTCTTATAAAAT
AAGTTTAAAACATAATATTCAGAAAATTAAAATTTAAAACTACCTTGTTTGAAAAAAAAAGTCGATGTTA
TATAAAGGAAAAAAAGTTGTTAGAAAAATTTAAAGATTTAAAAGGAATGTGATATTTACTAATTTTCCAT
CATGATATACAAAAATATGTTCATTTAATGGTAAAGAAAAAAAACAATGGTTCACATGCAAAACATGTGC
CAAGTACTACTGACGTGCAAGTTGTGCGAGCATAATTTTAGTCATGTGTAAAAACCAGCAAATGTCATCT
CCTAACAACGACAAAGTTAGAAATTATGTGCTGGTGGCTAGCAAATATATTAAGAAATATATATATATTT
TATGGATCGACTTATTAAAATGCTAAAATGTAGATTAAATAAAATTTGGCCCAAATGACGAAATATCTTG
TAACTTGTTTTGTTGAACTTGTATGTTTATTTAATTAAATGAAATGTTCAAAACATAGTTTTTTACCATA
TATATCTAGCTAGTATTTCAATACAAAAATATAATATAAACATGTTTAATTGAAGAAATAAATATATAAA
ATAAGTTTATTTCTTAAAAAAAATAAGTGAATTTTGTTGTAGATGTAATTTTTATTTTAATTATAGCGTT
TGATTTTTATCTTAAAAGAGTAATAAAAAACCAACCAAGTTTTTTTTTTTTTAAAAAAAAGGCAAAATGC
AAAGATAGCACTAGTATGTTATGATATATAAATTAATAATGAAAATACATAAAAAAATTTACTATTTAGA
ACCAGGGGTGATGGGACCTATACAGACTTAAAATATATTTTCATAACAATTAAAAATAAATAAAATCATG
GATAGGATGAAATTAAAAAAAAAAAATCTTTAGATTCTCAATATATTTTATAACTTTCATGTTGAAAAGG
CCAAACTAGGAGACTTTAATTGCATGGCTATCAATTTAACTAGAACTATTTTTATTCCTTTTATTATGCA
CGTAATTTCGCATTTTTCATGTTGAAATGTACTTTTAATCAATTGAAAGGCTTCAAAAATTTAATCATGC
ACATCCATGGGCTGGGCCTAAAACCCACTTTAGGTCACTACTCCTAGTCCTAGAAGTGTTCTCACAAAAT
GAGCCTGGCCCATTTCAATTCACCTCGATGGGATTTTCATCCACTATGTCCAATAAATGCAGTTTTTGAA
ATTTTTTAGGTAGTAATTTGATAAGATACATTTTTTTAGGAATAAGATTAGCATTACTACTCATAATTTA
TTGGGTATGCAGAGTAATTGGAGAACCAAAAATTGTTTCTCTGTACGACAGCGTTTTATAGATGCTGCTA
ATTCACTAATCTCTCTATATACATGCAGCCATGCAATGGTTATTGCCTGTTACCCCAAGAGTCACATTGT
GCCAAACACAAACCCATGGATTCTCTCCAATCTCTTCAAATTTCTCCGATGCTTTTTTTCATCTTGGTCT
CTCTATCTGTCTCCATAATTGTACTGATTCCG
Sequence ID: 79
Sequence Length: 2612
Sequence Type: DNA
Organism: Poplar sp.
CTATTTAACAACAAGGGACCATTTCGTGCATGCAAACATCAGGTAGGTGTTCGACACCTTGAGGAGAATTAATT
TATTATCTAAGTAGTAGTCACAATCACTACAACATTTAATAGTTTTACCGACATAATTTTTTCATCGGTGTAAG
ACACATATTTCATCGATAATTAATTTACCAACGAAATTACCGACAGAAATGCTTCTTCGGTGAATCTTTCATCC
GTAATTTTTTGTCCATCGGTAAATCCATTGGTAATAAAAAAAATATTATTACCAACGGATTTACTGACGGAACA
GACGCGTAAAAATAAATTACCCGCTTCATTCCGTTGGTATATCTCTCAAAAAATACCATCGGTAATTCTGTCGG
TAATTATTTAAAAAAATCCATTTTATAAAATTATAAAATAATTAAATTAACATAAATCAACACTCTATAATATA
TACTCAAAATGCTTGGAAAAAAGAATAAGAAAATCAACTCAAACAAATTTGCAACAAATAAAAATCTCACAGCA
ACATTTATACAATTATTAAGAACAAAAACAATTAAAAATAAAACAAAAAACAAATATAGTGAAAAAAATCATGG
AAAATGAAGAAAAAGGAAAAATCTTACCTTAATGTAGTTGCATGTGAAGCTAAGGAGAGAAAAAAAATTTCGTA
AAGCATATTAATTAAAAAAAAAAACTAAGAGGATAAAAGAAGAAAGAAGAAGACATACCTGAGCATGAAGGAGA
AGAGAAGGAGATGAGGATAGAGGAGAAGAGAAAGAAATAAATATTGATGTTTTTTACATATAGTGAAGAAGAAG
AAGAAGAAATGAGTCTGTCTCTTGTGAAATAAGGGGATTCAGGCTTTTTATTGAGGCGCGTTACCGACGGATAA
TTAAATATTAATATTTTTTAATTATTCTGTCGGTAATTCTGTCTGTAATATTTAATTTAAATTTTCAATTTCAT
AAAAAGTTTTCAGAAACCGCCAACAATCATCGATGATTTTTCAATCCGTTGGTGATTCCGTCGGTAATATTTAA
ATGAAAAATTTAAATTAATTGATTTTTTTCAGAAAACCACCAAATAACATCGACGACTTTTCAATCTGTCGGTG
ATTTTGTCTGTAAAGAACAACAATTAACAGTGCAATTGGAAAGTGAATAGTTCTAAAGCTCTCTGGAAAATACT
GACGGAATTATTCGTTGGTGATTCTCTTTGTAATTGACATGATGAACAGTGTTCACAGTTTACCAACGAATTTA
CCGACGGATTCCGTCGGTGATTCCCTTTGTAATTGACTTGATGAACAGTGTTTACAGTTTACCAACGAATTTAC
CGACGGATTTTACCGACGAAATAAATTTTGTTGGTAAAAATAACACGTCATCATTTTTTTTTTTGCTTTATTTT
AATTTTTTTCCCTACTGTAATTCCCTCGGTATTTCCGTTTGTATTTATCAATTTTCTAATGGTGAATTTTCTTT
AATTGTATCTTGATCTTAATAGAAAAATATAACAAGAAAAAAATTGGAGAACTAATTCGCTCGTTAATGAAATT
CATGTCCTGGTTAATGGGTGATTTGAGAAACTAATTTTTTGGTTAATGGGATCAATGAGCAATTGGGGAAACTA
AGTTTCTAGTGAATGAGATTAGTGTCCTAGTTAATGAACAATTAAATAACCAATGATTTAGTAATAAAATAAAA
TGATTTAGTAATAAAATTTATGAATCTTGATCGATGATGGCCAGCTGTTAGGTTTTATAATAAAGGATTACTTG
TGAAATGCTCAATCACGACTAGAGCACTAACAATAATTTTTGGGATTAAAAACATGTTAAAGATTGTGATTCAA
AATATTTTTAATTTACAAATAAATTAAAATATTATTTTTTAATTTTTAAAAATCATTTTAATTTGTTTCAGAAT
ATCAAAATGATATGAAAATATATAAAAAATAATTTTTAACTAAAAAACAAAAATTTAAAAATTTAAAAAAACAT
AATTTGCAGGTATTTTTAAACAGGGAAATAAGCTTTCAGGTCAACGTACAGAACAAAGATTAGAAATTGGTATC
TCAGCTGTTCCTGGCTTCCTACCAAAAGCTGAAATTAGAAAGAAAATAATGTTACCGTTTCCGTTCACTTGAGA
GCTATGATTAAAAAAACTGAATCAATCTGTCCTTTTCACTTAGTAGGATATGATTAAAAAAAACAAAAACAATT
TTTTGACTCTTTTTTTCCTCTAGTTTTTCATGCTTAAAGAGAAAAGCTAAATAAAAAGCCATTATACTATTTCT
ACCAAAACATGATTTATTGTAATAATTTTTTTGACTAATTTTCTAAATGAATTTTTAACATTAGAGTGCATATA
AACAAAAATAAAATCTCACTAAATTTGTTTTTCACAATTGATCCCTTTCTTCGAAAGAAAAAACAAGAAGCGAT
ACAAATAAAGACATTGAACTTATCCCCAGTTTGTCTTCTTCCTGTTATCATCTCCCTATTTACGACTATATATA
TATGTAGCATCTTCTCTATAAGAAGCAAGAGTTGGATTTAGGTTTGCTTTCACTAAGATAGAGCAAAGAACCCA
ACCTTAATAAATCAGACACAGC
Sequence ID: 80
Sequence Length: 3000
Sequence Type: DNA
Organism: Poplar sp.
AAACTTGATTATGTAAGGTAAATTAAAAACAATTAAGAATTGATGTCTAAATTGTAAATTCTAGGTTAGG
GTTACAAGAAAATTTAGGGGAAGTGAGAGATTGGCCTTGTTTGATGAAATTGGGATAGAAATAGACGAAA
GCAACTTAGAATAATAATTAAATGAAGAAAAATCATAGTCTTTTGGAAGCTAGTGGTGCCAGCCAAATAT
TAAAGAAAAATTAAAATAATACATGAATGTTGTTTGTTTGAGGTTATTGTAGCACGTGTTGAAATGTGGA
GAATATAATTTACAATAAATAGAAATCTTGTGTTTAATTTGTAATGGAAATATTGGTTATGCTTTGATTA
TGAATGTTGTCATTTTGTTGAGAATTGTAGGATTTTGTGTAATGTTGAAGTATTATGGAAAATTATACAA
ATGAGAAGAAAACTATTCATATTCCTTCTTTAATGTTACATTCGGCTAGAGAGAGAAAGAAAAGAGAAAT
TGTCCAGAACAAAAAATAGTCTAGCTATTTTGAGAAACGACTAGACCTATTTAAGAACACGATAAGACTA
TTTGTCAAAATGATTAGACCGTTTTTGTTATTGTCCAGTTTTGGAATTTTTTTTATCTTTTTCTAGTTTT
CTTCAGTTCTTTTGAGTTTGTTAAGGGAGATGGTAATCGTTTGATTGAATTTAGCATAAAATGTATTTTT
AATTATGATGGATGAAACACTTGATGTGAGGTTGTAACAATTATACATGGTAAAAATTTAGAATTTTCAA
TGCGATTAAATTGAGAATTCAAAATCTATATGGAGAATAAAATTGACCTTTATGGTGGATGAATTTACCA
ATGAAATCACCGACGGAAATGCTCCGTCGGTGAATCTTTCATCGGTAATTTTTTGTCCGTTACTAAATCT
GTTGGTAATAAAATAATATTATTACCGACGGATTTACGGACGAAACAGACGCGTAAAAAAAATTACCCGC
TTCATTCTGTTGGTATATCCCTCGAAATATACCATCGGTAATTATTTAAAAACATTTTAAAAAATCCATT
TTATAAAATTATAAAATAATTAAATTAACATAAATCAACACTCTATAATATATACTCAAAATGCTTGGAA
AAAAGAATAAGAAAATCAACTCAAACAAATTTGCAACAAATAAATAATCTCACAGCAACATTGATACAAT
TATTAAGAACAAAAACAATTAAAAATAAAATAAAAAACAAATATAGTGAAAACAATCATGAAAATGAAGA
CAAAAGAAAAATCTTACCTTAATGTAGTTGCATGTGAAGCTAAGGAGAGAGAGAAAATTTCGCAAAACAT
ATTAATTAAAAAACACTAAGAGGATAAAAGAAGAAAGAAGAAGAAGACATACCCGAGCATGGAGGAGAAC
AGAAGGAGATGATGATAGAAGAGAAGAGAAAGAAATAAATATTGATGTTTTTTACATATAGTGAAGAAGA
AGAAGAAGAAGAATGAGTCTCTCTTGTGAAATAAGGAGATTCAGGTTTTTTATTGGGGCTCGATAGTGAC
GGAATTACGGACGGATAATTAAATATTTTTTAATTATTTCGTCGGTAATTCTGTCTGTAATATTTAATTT
AAATTTTTAATTTCATAAAAAGTTTTCAGAAACCGCCTACAATCATCGATGATTTTAAAATTTAAAAAAA
CATTGTTTGCACTGCGTTTCTAAACATGCCCTAATCTTGTTTCTGTCCAAGTCGATGTCATTGCCCTTAC
AAAATAAAAGAAAAGGCAGTCAGCTTTCAGGTCATTTTCATTCCAGCAATGTTCAGTTAATTTTGCCATA
ATTAAGTTCCTGTCTCTTAATTCTTTTTTCCTTCGTAATTTATAGGAACCTGAAACATTGGTTTAGAGGA
TAAATTGTGTTTGGTAGAGTAGATATTATTTTCCCTTGTCTGAGAGGATGGAAAGGGAGCTACGAGAAAA
TTGTCGTCCATGACTGCCTTTTTTATCTCCCCGAAATGGATAGAATGAGGGAATGATGATATGGTACTTT
CTAGCTCTAACCCTTCGTTCTCCTGTAAATCAAAGCATACGATATAATTTTGTTATTTTTAATTCCACTC
TCTTTTTCTTTTTTAATTAATTAAATATAGGCAAGAACTGGACACTTAATTTACAATTATTTATAATTAC
GCTCTTCCACTTATATGAGAAAATTTTAAATATAGGTTTTTTGCGTTTTAAAAGCGATTTTAAACAAATT
AAAAAAATAATTGTTTTCTTTACTTTAAATTAATATTTTTTTTGGTGTTTTTAGATCATTTTGACGTGCT
GATGTCAAAAATAATTTTTAAAAAATAAAAACATATTATTTTAATATATTTCTAAATAAAAAAACAACCG
CAATCAAACTTTAATTCACGCGTAACCCATGTCAACGTACAAAACAAAGATTAGAAATCGGTATCTCAGC
TGTTCCTGGCTTCCAACCAAAAGCTGAAATTATAAAGAAAATAATGTTACAAGGGTCTCAATCTGTACTT
TTCACTTAGTAGGATATGATTAAGAAAAAAAAAACGACCTAAAGTAGCATCACTTAGTAGGATATTATCA
CTTAGTAGTTTAATTTATCATTACCGTTTCCGTTCACTTGAGAGCTGTTTATCTCTCTGGTCTAGCCTTA
GAGCAAATTGGATTTTCTAAATAGTTTTCTACCATATTAAAGTGCATATAAACAAAAATAAAATCTTACT
AAAATATGTGGTTTGGCTTTAGGTAGATAATAAAATCTCACTAAATTTGTTTTTCACAATTGATTCCTTT
CTTCAAAAGAAAAAACAAGAAGCGATACAAACAAAGACATTGAACTTATCCCCAGTTTGTTTTCTTCCTG
TTATCATGATCTCCCTAGTTACGACTATATATATGTGTAGCATCTTCTCTATATAAAGCAAGAGCTGGAT
TTAAGGTTTGCTTTCACTAAGATAGAGCATAGAGACCAACCTTAATATATTAGACATAGC
Sequence ID: 81
Sequence Length: 3000
Sequence Type: DNA
Organism: Poplar sp.
ATAACGGGAATGCCCATATGTCATGCACTCGGTCAGCTCAGCATTTTCAAGGTAGTATAACATGCAGAAG
TTAGGGCACATGTCAATTTTTTAGTATCCTAAACTGAGGGGTTTCATCATGGACTTGGCAGCATAGAAGT
TCTCTTTTAGCCTGTTCCCTTCAGGTAAAATGCTTCTCACCCATTCGATAATCTTGTCATAACCGACCTC
ACTCAACCCGTGATTTAACTTGATGGTGAACACATGTGCTACGGCTGATAATTTACTGTGGTTCGTGCAG
CCATCCCATAATGGTTCGTCAGAATCTTTCAACAGATCAAAAAACCTTGCTACATCTGTATTACGTTCTT
CTTCTATGATTGGACATTCACTGACATTACCTTCACTCATTCTCATTGCATCCATAACCATATTCATGTA
AGGATTATTGTTCTCATTTCCAATTTCATGCATATTGCTAGCACTAGAAGTTGACCCAACCACTCTTTCT
ACCATGCTCTTATTAGGAACAAATAGTTCTCTATGTGCATAACAACATAGGTAATCCTCCATGAACCATT
TGGTTAGAAGATGCATCGTTACAACATCTTGATGCAGAAACTTTAAAATTTTACACTTCCTGCATGGACA
ACTAATACCGCCATCAGCAAAATTCCTCGGGATAGATGTTGTGAAATTAATAAAACCCTGAACCCCGTTA
CAATAATCCATCCTCCGCAATCCTTGGGGTGAGTCCCAATACATCCATGAACGATCATCCATGACTTCTA
TCGAACCTCTATAAAATTATGACGACAAAATGTATTAATTAACTACATTACGTAAATAAACATAACAAAA
ATATTGGTTTTCCCCGACATTATCCAACAAACTAAAAAAAAAATTATGTTAAATATTCATTATCAATTAT
CAACTATCAACGTCCATACAAAATTTATACATATATAAATTTACGGAAATCACAACTTCGCAATAGAATT
CATAACAATCAAAACAGACCCGTTAAACAAGCTGTTATTTATTTCATCACAACACATCTAATTCGGTGTA
ATTCAAACAAAGTTTCAACAATTTACAAACAAATATAATTTGACAAAATCTAAAACAAACTATAACAACT
ACAAAATTATACATTCATACTACATGTTTCTTTGACAAATAACAATCAAACAAGTACATACGTTAACAAA
ATACATATACTAAAAAAACAATAAAATTCTCATTTAAATGTTAAAACTATATAAAAAAGGATAAATTTAC
TTACAAAAAATGATAAAATCCACAAAAACGGATGTTGTGACCACGTACAAAATTATAAGAAACTTAAGTG
CTTGTGTTGAGAAGAGGGGAGAGTTAGGATAGGTGGTTGGTTGCAGTTTGGAAGGGGAGAGGTGGGATGG
GGAAGAAGAAGGAGAAATAGAGGAAGAGAGTGTCAGCAGAGTGGGTATATATTGACTTTTACCGATGGAA
TCACCGACGGACACGTTCCGTCGGTATACCCGTCGGTGATTCCGCTGGTGAAAGTGTTACATCACTGTAC
GGATATCCCAGTTTGAATCCCTCGGTCATTCCGTCGGTAAAATCGTCTGAAAAAAATCCATGCCATCACA
CCGTTGTAACTTTTCAAAAAAATTGTATAGTCCATCGGCGATACGGTCGGTATATACCGACGAATGTATT
TCATCGGTATATAACGACCGTATCGCTGACGGATGGTATCTGTAGGTATATACCGACAGATTTTGAGACG
GAATTATATCCGTCAGTAATTCCATTGGTTTTCGCCGATTTTCTCATAGTGTCGATTGTATAAAATTTAG
GGTTGTTACACGACAAATCAACTATATCCAAGGAGAAGATAATCTTATTAGAGATAGGGAGAAATAAGAA
ATTACATATTTGGAGGAAGAGAATGATCAACTCGACTCAACTCCCAGGTTTTCACACCCATCACTTCCCT
TACAGTTGTTGCAAATAACAGCTTTAGTTGCTGCCCTCAGCAGCTCCTGAAAAAACTTTACTCTATAAAA
TGAATTTCTCTAACCCTTCGTTCTCCTGTAAATCAAAGCATACGATAATTTTGTTCTTTTTAATTCCACT
CTCTTTTTCTTTTTTAATTAATTAAATATAGGCAAGAACTGGGCACTTAATTTACAATTATTTATAATTA
CGCTCTTCCACTTATATGAGAAAATTTTAAATATAGGTTTTTTGCGTTTTAAAAGCGCTTTTAAACAAAT
TAAAAAAAAATTGTTTTCTTTGCTTTAAATTAATATTTTTTTTTGTGTTTTTAGATCATTTTGATGCGCT
GATATCAAAAATAATTTTTAAAAAATAAAAAATATTATTTTAATACATTTCTAAATAAAAAAAATAACCA
CAATCAAACTCTCATTCACGCGTAACCCATGTCTACGTACATAACAAAGATTAGAAATCGGTATCTCAGC
TGTTCCTGGCTTCCAACCAAAAGCTGAAATTATAAAGAAAATAATGTTACAAGGGTCTCAATCTGTACTT
TTCACTTAGTAGGATATGATTAAGAAAAAAAAAAACGACCTAAAGTAGCATCACTTAGTAGGATATTATC
ACTTGGTAGTTTAATTTATCATTACCGTTTCCGTTCACTTGAGAGCTGTTTATATCTCTCTGGTCTAGCA
TTAGAGCAAATTGGATTTTCTAAATAGTTTTCTATCATATTAAAGTGCATATAAACAAAAATAAAATCTT
ACTAAATTATGTGGTTTGGCTTTAGGTAGATAATAAAATCTCACTAAATTTGTTTTTCACAATTGATTCC
TTTCTTCAAAAGAAAAAACAAGAAGCGATACAAACAAAGACATTGAACTTTTCCCCAGTTTGTTTTCTTC
CTGTTATCATCTCCCTAGTTACGACTATATATATGTGTAGCATCTTCTCTATATAAAGCAAGAGCTGGAT
TTAAGGTTTGCTTTCACTAAGATAGAGCATAGAGACCAACCTTAATATATTACACATAGC
Sequence ID: 82
Sequence Length: 3000
Sequence Type: DNA
Organism: Poplar sp.
ATTATTTGGGTCAACCTAAAGCTCCTTTGACCCAGCTCTTTGCTTGATATGAATTTCAAATTAAATTGTG
TGAAAATTAAAATAATATGACTCTAATGATTTGATTAGTCTAATTATGATCTAATCGACCTAGTCTGAAA
CCAACTTTAACTAAAAAAATTATAGACTTTATTATTTTTTATATATATAACTATTAACACGAGGACACTC
TGAGTGGACTCGAGTCTTAAGATGTGACCTAGATAACTCGGTCAACCAAACCAAAACTTAATTTGCTTTT
AAGAAGAAATTTAAATGAACTTAATCCAACGTTTTCTTTAAAGAAAATTATCCTAAAATTTTTATAATTT
ACTCCATTTTATAATATATTTTTATAATTTTAAAAATTATAGTAAACATCCCAAGTTTTATAAGTTAATA
ACATTTTATACAGAAAAATAAAAGAATCAACAAAAAAAATCTTATCAAGTATTACATGAAGGAAGACATG
GGTGGATTTTTTGTGGCCCTGAATTAAATGTGAGAAAAATATACAAGAGAGAGGACAAATAATATTAATA
TTGAGCCCAGTTTGGCTTACTGTCTCGCCGTGATTAGAGAAACATCATCATATGCCAAGTTAAAATCATA
GGCTGATGGTCCAGCAGTTTACTGTAAAATCATAGACTGGGTCGAGCATCATTATTATCAATGCCTGCAA
GTTCTCTGTGTGGTAATTTTTTTGTTTTGTTTTTAAAAGTAATTTAATTATTTAACTGTTTTTTTTTAAA
TGAAGGATTAAGATGCCTCTAGATCCTAAGCTAATTTTTTTTTTGTATTTTAAGGATAATTAAATCTTTT
TATTGTAAAAAAATAAAATAAAAAGAGTAATTTAACCTCAATAAATTTAATAATGTTAATCAAATCTCGT
GAAAAGATTAATTTGTGCCTCAACTTGCACTTCCTTGACTAGTACCATGGAGAGCAAAACCATAATTGAA
CTGTAGCAATTCACAATATACCGAGTCTTGGTCTACCGTAATTGACTTAGCCTCATTAGTTTTTTTTTTA
TTATTTGATTTAGTTTTTTAAAAAATAGGTTTGTTTTAAACAATAGTGTATGTTATTTTTTATTAGATTT
AAAAAATTAAAAAGTATTGATATTTAGAGTTTATTAAATCATATATATATATATATATATATATATATAT
TTTTACATTTCAGCTGTCATTTTAAGATAAAGAAACCATAATATTTTTCTTTTCACTGTATGCTGATCAT
AATCAAATCGAGAAAGAGAATCGCAATAACTGGCTAGACTAAATTCATAATCAAATCGAGAAAGAAATTC
ATAATCTAGGTCATGAGATCAAGATAACCCCATAAAAATAAAATAAATAAATTATAAAACCCCACTTTCA
ACAAATTCAATGTTGAATGTTGAAATCGAAAGAAAAAAACTAAATCCATGAGATAAGGATAATACAACCA
AAAGAAAATAGAAAAAACATTATGAAACCCAATTCCCAAACAACCTAATATTATAAAGAAAAAACTAAAT
AAAAAAATAAATGAGTTGTTGAAAGGTGAAATTGAAAAAAATAATAATTAAAAAGGACCCAAAAAAATAA
ACCGTGTCATCCCATGTCAATCTACCAAACTTGCGATCCGGGTTATGAGTTGTTGGAAGGTGAAATTAAA
AAAAATCAATTAAAAAAGAACAAAAACAAATTAATGGTTTTTTTTAATTGTTTCCCAACAAACGATGCCA
ATTGATAATGTTTAAAAATTCGATATGCTTTGGAATAAAGTTTTTTGGTGGTTGAATTATATGTTAATTA
AATGTACATTCAAATGACTCTTTTTTTCAAAGTGAAGGAAATTATCTTTTGATCATAAATACCCCTGGAT
ACCTATAAAAAGTTCTTTTTTGGGGGATTTAAGGATCCTCCGATGGTAAAATAGGTAATCTTATAGAGAA
AAGGAGTAATAAATATGCTTGAGAGCCTTAAATTTTAAGAACAAAGTGATTTTTGTGTTGGATATATATG
TTTTTTGAATGTAAAGTCTTGAACCTTTCACCTAGATAGTTCAAGGGGTATTTATAACCCTTGAACTTTG
GCTTCTTCTTGAGTGGAGGAGTTGGAGAATAAATTTACAATGATAATATTTATAAAAAATAAATATCTAT
TACACATACATTAATAAGAGAAGATTGCATTAATGAAGGAAGAAGTAAAATGGGTTTAAGACACTCACTA
TGTCCAAGAAAACTAGGCCATGTTAATGGGTTGAAAAAATAGACAATGACCCAAAATGACCAGGCACAAC
CCTTGGGATTGGGCTCGGGAGCACCACTCGAGCCCAAAAAATGTTGCGCTTGCGCTCAACATTGGGCTCG
CGTTGTGCACTCAAGCCCATAACAAGGTGCTGGGGTCAGGTATGCAACCCGAGCCCACAAGGTGTTAGAT
ATGAGCCTATTAATTGGGTTCATGAGTATTAATGGCTTATGCTAAAAGTAAATATCTTTATATTAAATAT
TAAAAATGTTTTGATGAATCTTGATCCATCAACAGTGATTAAAAATGTCTGTTAGGTTTGTATCAATTAA
GGATTGCGTGTAAAATACTCAATCACGACAAGCGCACCAACAATAATTTTTGGGATTAAGCTTGTTTCTG
TCCTAGCTGTTATAATAATGTTATAAGGGTCTCAATCTGTCTTTATACTAACATTGATGTCTTTTTCTTA
ATTGATTTCCTCCTACTAGTAGAATTGGTGGAACCAAATTGAGACAAGTAAATGAGTTCAGATATCCACA
ATTGATCCCTTTCTTCAAAATAAAAAACAAGAAGCGATACAAACAAAGACATTGAACTTATCCCCAGTTT
GTCTTCTTCCTGTTATCATCTCCCTAGTTACGACTATATATGTGTAGCATCTTCTCTGTATGAACCAAGA
GTTGGATTTAGGTTTGCTTTCACTAAGATAGAGCATAGAGACCAACCTTAATATATTAGC
Sequence ID: 83
Sequence Length: 3000
Sequence Type: DNA
Organism: Poplar sp.
TGGAGATGTACACCAGGTCTGCTCCTGTTGCCTATTGCTAAGACTTCACAACTTTAATACAGCACATAGG
GTGCTAAATCTTAATTAGCATGGTAACTGGGTTGTTCTGCATATATAATTAGAAATCTTAAATTCTTTCT
CTGGAATTGCTTCTATGGACATGCAACACTGAAGAACTAAAAGGAAACCTTGGAAAAGAAACCTGTCATC
ATTGAACAGGAAGTATTTGTTTCTGAAGGCTTTCTTTTTAGTACATAACTGGACAATGCCTTATTTGGGT
CTCTACTCCTTTGGAGTGCTTTTGTGATATTAGTAAATACTTTATTTGTTGTGGTTTATATGTGATGATG
TTTAGTAATGCTCATCTATTGGCTGCAGAGCCATTAAACTTGACTCACTTGTCTACTGACCTGATTTCAG
AACTTGTTTTGTGATGACAGCCCCTGCATGTAGGTTTCCTTGGAGATTTGGGAGGAAACACAATACAAGT
CCATTGGTACCGTAGGAAGTCAAACCTGCACCATGTAAGCTTTTCTGTAGTGTGTTAATCTACACACGTG
GAGGATGATTCCCGTATAAACAAAGAGAATGTTTTGAGACAGTTTATGTGCATGGAATGGATTTGGATCC
TCTCATGTTCTTGAGAACATGATGAATCGTGTTTAAATTCAAATCAATAATTATATTTTAGCTTAAATTC
AAGTACCTTGCTAATTATATATCTGAAAACTATGTAAAAGATCAAGTTTAAAAACGATTCATCATGTTCT
CAAGAATTTGAGAGTATCCAAGTTGGTCTGTCAACAAGCAATATATAGCAATGCAGTCAAGGCAACTGAT
TTCTTTTGATCTAAATAGAAAAGCATCGTGGACTTTAATGACCATCAAATTACTTTGACATGACCTTTTA
TGCTCATTTTGTTCTCTTCACCAACTCAATATGGTTTCCAAATAATGGTCATCGTTTATATGTTTCTGGA
ACAACAAATGCTCTTTCCCATGTGTTTTTGGATGTCATTAGCATTATATAGTCTCCTATATCAGTTTCAA
TATTGTTACTCTCTCTCTCTCAGGTATGGGACAACATGATTATTGAATCAGCCTTGAAGACATTCTACAG
TTCAGATCTTGCGACCATGATACGGGCCATTCAGAATAATATAACAGTATACTTTTCCTTCTAACCTATT
TCTCTGATTTATAATTGGAAAATAGCATGTACATTGGTATCAGTAGTGTTGAAGGACAGTACTCGATATA
TCTGCTTCTTTTATTGTTCTGAATTGTTCTATATAAATCACATGAAAGTTATAACTTGGGGATATGAATT
TGTTGCTTCACAAATTTTTATTTTGTGCGAGTGCATGCTCTTGGATTTTGCTTTTGCTTTATTTATTTGC
CATTTTTTTTAGTGAGAAGCTTTTTTTGATTGCTTTATTTCATTGTTGATAGGAAAATTGGTCCAATCAA
CAACCACTATGGGAGCATTGTGCACACAACCACACAGTCTGTCCAAACCCGTAAGTATATTCCTTTTCCC
TTGCATGCTTCATCTGCGTAATGGCAGACATGTAAATTGTAATAGTGCAATCAACCATGCACTGAAAGGG
CTATTTTCGGAATGGTGAAAGCTTTACTCGTTTGAAACTGCTGAAACACATAAGCATGTCAATTCTCCCA
TACAGAGTTAGACTTGATTGCTCGAGATGCAAGTTTTTCATTGCATTCGGGGAATAAATTCACTTCATCC
ATCAGTTGCAAGGAGTTGGTAACTAATGGCTTGATCCTTTTCCCTTCAAATTGTGCAGCTATGCTTCTGA
AAGCATTAGTTTGGCATGCAAGTTTGCATACAAGAATGCCTCGCCTGGAAGCACTCTGGAAGGTATTCTC
TTCAATCAAACAATCAATAAATTTTAGATTCTTGTTACAGAAGCTAGATCAGGGTAGCCTTGCATATGAT
TAAAATGTGACTCATGTAAAGCTTATTCTCATGAGTCTTGCCTGCTTCCTACTGGTGCAAAGCGTGCCCT
GTAGGTTGTTGATATTGGTTTTATTTTATTCCAAGTAAGGGGGGAAATGCCATTTTTGTTATCACAACTA
ACCTTGAAAATGCTATATATTTGTGTTTTTTTACGTGTCATTTTATCCCATGAATCATTTTATCTCTATG
CTCAAAGCCGCAAAATCAGACCTTCCAATATTTGATTGGACAATGCAAAGGTCTTTGTAGTCAACACAAT
ATCGTATGGACGTTTGGACCATGCTTCTCATGTATTTAACCATGCACATGCCCAGTTCAATCCAGCATGT
TATGCATGTGGACTGCGGCAAACTCAATCCAGCAAGTTTTTTAACAAATAGACTGTTTTCTCACGTCATA
AACTACCTGAATTTCAGCCGCACTCTGTTAAGAATAGTTTTTAATCCTTGATCTTTTGCGCTGCAGATGA
TTACTTTCTTTCCCGGCTGCCCGTTGTGGAAAAGAGACTAGCCCAAGGGGGCATTCGGTTAGCTGCCACC
CTGAACCGCATTTTTGCTTCTTACGTGAAAATTGCTCAAGCATGAAGATAAAGAGAGAAAGAAGCATCTT
CAGTTGCTTACACAGAGCGGGGACCACCGCTCCAATTTATATATAGCACGAACTTACCTTATGCGTAATA
CTTTACTCATTGTCAGCTCTGTTGAGCAAGCTCCGAACTAATCTTTTAAACCAATTCTTGATTTCATTCC
TGTTATGCGGTACGCATTGTCTTGGATATTCCCGAATATGCCCTTCTTGCACATGGAAAATGTCAACTTG
AAGGGTACTCAAGAGTGGAATTTAATGCTTGTACTCAATAAGAACCCTTCTTTCTTTATTGTGTAAATGT
AACCGAACCTTTTCTAAGCTTTTTATATATGTATCCAAAACTCAAATTAGTGGCTCATTTCGTTTCTCAT
GTTTTGTGATATTGGAGGAAGAAAACATCAAGCAGTTCAAGCTGAGAAAAAAATAAAGAT
Sequence ID: 84
Sequence Length: 3000
Sequence Type: DNA
Organism: Poplar sp.
ATATATATATATATATTTGTTTTTTTGGATCGAGCGAATAGTCCAGCATAAAACACACAAATTAATTGTT
TTTTAATGGCTATTTGTAAGTAGAGATTTCATAGTTGTTAATAATTTTAAATGATATGTCCAGCGATCAG
TGACGAGGTTGTGAAAAATAATACGACCAAATCATAATTAACGAGCACCCCATCAATTCAACTACTTTTG
GGTTTTAGGAAATTGGGAAAAAAAAGGTTGAAGAAGAAAATTCTAAAAATAAAAAGAAAAAGACAAGTCC
CTGGCCTTAGGGCCACTAACTATTTTGTTTTATTTGATCCTGAATAATTTTTTTTTTTTTTTAGTTTATA
TCAGCCTTGTGGTTGCTGCAGCTCCCTCCAATTTAGATATCATCGAACAGATGGCACTGTAATTACTGGT
ATTAGGATTGCGACAGCACACCATACATGGCATAAATAATCAGCTAGCGTCATAAAATCAACATTAAAGT
CAAATTAATTAGTGAACGATTTATATTGTTTCAGATAATTAAATAAACTTCGACCAGTAGACATATTTAG
GAAATTTATAGTGGATGAGGAGGAGGAGGTGGGGGCAAAGAAGACTTCAATGGCAGCACCCTACCAACCA
CAATAAAATAAAAATAAAACGGGGGTGGTAAGCAGGCTTTTCCACTGCAACCAGGCAAGTTGGTGAGAAC
AAGAGGAATTTGCAAGTACAAGAAATGTAAGAACCCAACCTATCACAACCCGAAAAAGTAAACGTTAACC
AATAAATCTTTTAATCCAATTATTTGACTGGCTCGTGTGTAAAATTAACCTAAGTAAATATTATTTTGAT
ATGAGTTAATCATCTAGAAATAACCTGGATAATGAATAAAAAGTATGGTTAGATTTTTAAAAAATAAATT
TAAAATAATATTTTTTTAAAATTAAAATGATAATATGTTGAATCTACTTAAATTTTATAGATTAATCTGC
TAAACTCGTAATTCAAATTATAAACTCCATTGAATCCAACAGCTTTATATTTAATGATACAATAATAAAG
GATAAATATTTTTAAAATTAAATAATAATAAAAATAGAAAATAATTATGAGAATAATTTCAAATATTAAA
GAATTAAAAAAGTGGAAGATGGATTCCAAGAAAAAGACAAAAAAGGGAGCAACACCTTTATTGTTCTTTG
TATCCCGGTTCTTTCAGCACAGGAGATAATAATTAAAGATTAGGAGTGTCTCTTACAAATACCCGAAAAG
TCTATTTAATATATTTTGCTAGCAAGCAAGCTTGCTTGATGGCGACCTATGAAATAATGACGATGGCTAC
ACGAATGTGAATCCATGGATGCTTATTGGCTAGAGTGTTTTCTTCTCTCTGCATTATTATTTTTTTGTTT
GCAATTGCTGGACTCTGTAGGATTCTGATGCGAGGTTTCAAAATATTATTATATATATAAGAGAAATACC
ATACATTTCATAAAAACAAACTGTACCTGTGATTTGCATTCTATAAGAAACAGTGGTGTGCAGTGCAGCC
CATGGGGGGCAAAGGGATCGCTGCAAAAAAAAAAAAAAAAAATGATGGAACGTCCAAGAGATAGGCTACA
GATGTAAAGGTATGCACAAAAAAAACCAAAATGGCTTTCAAATTGTCAAGTAATCAATGGAGTCTGGGAA
AAAGAAATGGCTAAAGAGCTCCACATTACAAGTGAAGAACGGCCGGCCATGAGTTGAATACTTCCTGTTA
ATGGTTACCTTGAGTTCATCAAAAAGTGATATTATTTTAATTTTATTTTTTTAAAATTAAATTTAGTTTC
GATTGAGTTATGAAATAATTCACCATGTTATTTGTATTTGACTAGATTATCTCCAGCCTGATTTTTTTGC
ACCCGGTCCGAGATAGTTCTAAATTTAGCCAGGAAGCACACTGTTCAGGCCGTTTTAACACGCAAGTAGC
AAAAAGAAGCACATGATTACAATTAATAAATTTTGCTCGAGTTTTCTTCTATAATATTTTTGCCTGGCAT
TGATCCAGCCGACATGATTCTTGGATTTACCATGCACTCGCAATGAAAAATCACAACTTGATATAATTAA
TTAATATAAAAAAAGAAATACCTAACACTAAGGCCGAGTTAGAATAATGCCCAAGTCCAATAGCTTGCAT
GCATCAACTTGAAAGCTAGGATGACGGGAAATTTTATAAGAAATAAAAGAGAAACTGTAGAAATTCAAAA
CAATCCGGAAATATCTCATGATATATAATTATCACTATTTATTTTTTAAAGGGAAAATAACTAACAAAAT
CAATCGAGTTACATAGCATTTCCGCGTGTAGATAAATAAAGAAAAAAAATGGATTTTTTTCTATTTTCAA
GTAAAGATATGCATGGATGTGTTCGTTGTAATTCCCTCTGCCTTTTAGGTAGATATGGTGATTTAAATTT
CATGGCAGAAAAATCATTGATTAAATAATAAGAATTAGAGCCATGACGTAGTGCTGATGTGCCACTTCTC
AACAACCCCCATTTCTCCCCAACCTTCTATCTTTTTGACAAGAACAGGAAATCCTCCGAATTATGGGACC
CAACTTAAGTGCATGCTACTTACCACAATATCCCAAGTGTCACAATCCCACCCATTGAATCCCATGATAA
TCCAACGGCCACGTACACTTCTCAACTAACACATCGGCCAACCCACCCCACCCTCTACCTGCCTGCCCCT
CCTTCCTTCTCTATTTAAAGCACCTAAATTTTGGTGAACAAACTCAGGATTTTTGGGTAACCTGAAAATT
CCTACATACATTTAGAGCCTCGCAACTTCAATTTTTTCTTACTATACAACCCTCATATAATTTTTTACGC
CAGAAAATTCTTTGTGCTCACACATCCGTTCTTCTCTCTCATTTTCTCTTTGATCATACCAATCGATCCA
TCACTCTTTTTTTGTCTCTCCCCCATTTTGGCATAGAGGATCATCGCTTTACAGGGAAAA
Sequence ID: 85
Sequence Length: 3214
Sequence Type: DNA
Organism: Poplar sp.
ATATTAAAACAACATGATAAGATTCAAGGGTTATATATATCATGGAACTACTCAGTAAATATATTATTTA
TATTTTTTTATTTTCTAAATATTTATAACCGCTAATAATATTTTTAAACGATATGTTCTAATTAATGATA
AGACAATGAATGCACTAAGATTTTATATATATATAGATAAACAGGTCTCTGGTCTTAAGTCCACTTTCTT
GTTTAATTTCCTGAATATTTTTAATCTGTGATTGTTGCAGCTCCAACTCAGATAATATCAAACAGATGGC
CCTGTAGTTAATACCGGCATTGAGACAGTGACGGCACACCATACATGGCATAAATAATTAGATAGCATAA
CAAATTCGACACTAAATCCAAATAAATTAGCGCTTAATTTATATTGTTCTGGCCTGGGTAGATGAACTTC
AACGAGCAGAGATATTCGGAAAATTAATTAATTATGAGGATGACGAGGGCTGTAGGGCAAAGAAGACTTA
GTGACAGCTTCCTACCAACCACAATCTAATTAAATTAGAACGGGGATTGGTAGGCATTCCACTACGCAAG
TTGATGTAGAAGTACAGAAAAGTAAGAGTCCAAGCAACCAATATTCCAAGCTCAAAATGCACACAGGCTG
CATCTTTTTCATGGTTTCTGGGCAGTAAAGAAAGGTAAAAGGGATGCGAAGTAATTCGTTCATCGTCCAC
GAGATCGCTACGAATGCAAATCAAGGGATGCACAAAGAATCCGACACAAGAGATTGAATATTAATTGGCT
GTCGGTCATGTGGTTCCGAGCTGTCAACTGATAAACTCTTAGGCTGAGAGATAAATGCACTTTGGGACTC
CAAATATCCAGTTATATTTACAGTGAATTCATCGCACTTGTCGTCTCTGTGCAGTCCTTCTGGACTTGGT
TTTTCCTGAGTTAATATGTAGGGTATTATCTGGTACTGACCTGGCCCTGACTTCTATGAAGCGTTTGGAA
ACGTGGCAGGCGGCGGCTTTTTAATTTTCTTTTCTGGAAGTAATATTAAAAATATTACTTCCAGACAAAA
AATACTTTAACAACAATTATCCCATACCATACAATGACTTGCTCATCCAAAGACCCAGCTGTCCATACGT
TAGACATCCATTAATAATAATAATAATAAAATAATAATAATGCGCTATATATTCTCCTAGATAAATCACT
TGCTCTTCCAACTCCCAGCGAAGCCTGTTCTTTTCTTTTGTGATAATTTTATTTTCTTGATTTAGCTATG
AAGCCCACTGCCAAGGCCGTTTTAGCACCTAAAATAAATCTGAAACCCGGTTCTGGTCCGGTTTTTTTGA
ACCAAAAAGGATACTGAACCCACAGCTCAGGATCATCATGCACATCATTGTAAATGAGATGTCTATTAAA
TTTTGAGCCACATGATAGTATTAAATCTTGCACTGTATCATGCACATCATGACGAGTAATAATAAAAGCT
TGAAAACCATTATTTATTAGAAGAGTTACATCTATATAATAGTGTTTGGGATAATATAATATTACTTGGG
ATAATGATTAATTAATATATTTAAATACTTGATTTTTTTTTAATATAATGAACTCCAGGCTAATATATAC
TGGAAGCTTGAATAACTGCAAAAACAGTCTTGATGGAAGGAAATTGAAGATTCTGTGATGATAAATTTAA
TATATTTATTAAAAATATATTAAATGATTTAAGACAAGAAATAATTTTAAAGAGCTTTAAATTCAAAAAA
TAATGATGTTTATTTTTAAGTTTTAAGTGGGTTAACACTCTGAAATTCATGTATTTTTTATCTTAAGACA
TGAAAAATTATGAACATTTTATCTATATAATTCAGGGGATACTTATACCTCTTAAACTTTGTCGTTTTCA
GATAAGTAAAATTAAGGCTAGAAAGTCTTCTGTTTTCGATGATATTAACGAGAGAAAAATATTTCTTACA
TATCATTAATGACATAGTAAGTATAGCAAGTCCCTTAAAAAAACCTTGTATACACCTATAAGTATAAGGA
AATTATTACATTAACATGAATGATTATTCTAGATGGAGAAGCAAGACAGTTTATCATGTTTTTTATATTT
ATATTGTAGATGATCGTCTAGGATCAAACATATAACTTTAGAACTTGGTAATATCTAAGTTTTTTGGGTT
TGAGATGTTTGATGGACATACATTTCCTTATACATGACTGACTACCAAGTCTAAATTTTTTAAGCCTAGC
ATATTTATCAGACCAACGTTTAACCCATATTTACCAGACTAACTGTTAAGTTCAAGTTTTTGGGATCTGA
CATGTTCGTTAAACTCACATTACCTTGAGCTTGTTTAACTGTCAAATCTAAATATTTTGGGTTAGACATA
TTTGCCTAACTTAATTTATTTAATAATATTTTTTATAAATAAAAATCTTAACCAAAAATTAATCCATCAC
TTTCAATATAGTTTCAACTATAAAAACGTTATAAATGACAATTAAGTCTTATTTTTATGAGAGAAACTGT
CAACTACACAACAATTTCAAATCTAATTGTGTGAGACTAGTAAATCTTAGTTAATGGATTCGCAATGAAA
CCTTCTATCTATAACACCGTGTCCTTGATTTTCTTTCCATTGTAGGTGGACAGCACAAGATACTGTGATT
TAATTTCCTTTTTTTTTTTTTGGCAAAAAAAAAATTGCTGATTTAAGTAATGGACATTAAAGCCATGACG
TAGTGCTTATGTGCCCCCTTCCCACCAACCCCCATCTCTCCCACCCTTGTATTTTCTCGACCGGAAAACC
TCGCCATTCTTGGACCCACCTAAGTGCGGGCTACTTACCACAGCATTCACGTGTCAAGATCCCAACCATT
GAAACCCATGATGATCCAACGGCCACGTGCACTTTTCAACGAACACATCGGCCAACCCACCCACCCCTCA
ATTAGCCTCAGCTCCCCTGCACACTCTATTTAAAGCACTTGATTTTTGGTGAACAAACTCAGGGATTTTT
GGTGACCTGAAGATTCCTACATACACATAGAGCCCAGCTATCTTAATCTTGCATGCTGAGCAGCCCTCTT
GTATTCTTACACACACGAGAAAAAGAGATATATTTTGAGCACACGTATCCATTTTCTCCCTCCTTGACCA
CCGATCAATCCATAATTCCCTTCTTTTTCCCATAGAGAATCATCACTTTCAAGAACGGGAGAAA
Sequence ID: 86
Sequence Length: 1880
Sequence Type: DNA
Organism: Poplar sp.
GAGAAAAATAAGCTTGACTAATTTCACTATGACCATATATACTATGTGTATGGAGTGAGCAGTGAGGCTC
TCACCAACCACACATGTAGCTTATCCAAGAGTTTGACAAATTAATTAAAGCCTGTAAGCTTGATATCTCT
CTATGATTTTCTCATTGTCTTCACCTACCCTCCCTGGATGAGAACCACCACTGTGGTTTGTGGTGAACAA
GACCCGGTCTAACCACAACAACTCCTGGCTACTATTTTCTTCTTCTTTTTTTTACATTTTCTTGAAGAAT
ATGTGCACTGTGGTGTTCTCCACTGATGTATCTTTTTTAATTGATGCAGGCTGCTGGATATTATCTACCA
GGAACCTCACGATACTGGGAGCTAGTAAAAGATAAAAATAATAGAGATATGATATACATAATTTCATATA
TTTCTTGTTTTAAAAATATATAAATTTATCTTAAAAACTGTTACCATAGAGACATGTTTATTTTATTTGT
GTAAATAAATATGTTTCTTCCTATCTTTTTTATTACGATTGCTCATAATAAAGACTTTATTATGAGCAAT
CGTCCATTAATTCAAGAGGTTAATGATCATATCACGTGTAATTAATGCTGAATTGATCATGGGAAACACG
TAAGATGTTTCCTCCTCAAACAGCAAACCTTAAACTAGTTGCTTTGAAGAAAAAAAAAAAAAAAAGGGTT
TGGATCATAAATTAACTTGACAGGTTTTTTACAACTCTACTCAGAGCAGGTTCAAACTATCGGGCAAGCA
GGGTTTAATTAATAAAGGTGGCTCTGTATGCTCTGCTTTTCTTCTCGAAAGAGAGAATGGTATGGGTAAA
CTTTCAGGATGAGAAATATATCCTTTTCTTTGTTGTCTTCGTAGGTTTTTTTTGGATTCGTTGATTTTTT
CTTCATTATGGCTATATCCTATATATCAGCCAGTGACTTGGCCCTGGCTTTCACTTGTTCAAACCCTACG
TTCGATATAAATATTTTTAAATGGTTAAACAGTAATACCAAATAATCAGGGCAGAACCAGGGGAGGGGAG
GCAAGCAGGAGCTGGGCGCCTGTAAAAGATTTTAGACTTTTTTATTATTTAGTGTTTAAATATAGAATAA
TATATTATTTTTTTATTTTAAATAGAGGGATTTTCTAATAATAAATTATATCTTGAGTTTTGTCTACACC
CCAACAAATATTATTTTAATAACGGTTTTTTTTTATAAATTTAGTCATCAAGCCCACTGTTCAGGCCGTT
TTAACACCCAATTAAGTAAATCTCAGTAGGACTAGCCGAAAGAAGCACATGATTACAATTAATAAATTTT
ACTTGAGTTTTCTTCTATAATATTTATGCCTGGCTTTGATCCAGCCGCCATGATTCTTGGATTTACCATG
CACTCACGATGAAAAGTCACCACTTAAGTGCATGCTACTTACCACAACATCCCAAGTGTCACAATCCCAC
CCATTGAATCCCATGATGATCCAACGGCCACATACACTTTTCAACCAGCACATCGGCCAACCCACCCCAT
CCTTCTCTATTTAAAGCACCTAAATTTTGGTGAATAAACTCAGGATTTTTGGGTAACCTGAAAATTCCTA
CATGCATTTAGAGCCTCGCAACTTCAGTTTTTTTTACTATACAACCCTCATATAATTTTTTACGCCAGAA
AATTCTTTGTGCTCACACATCCGTTCTTCTCTCTCATTTTCTCTTTGATCATACCAATTGATCCATCACT
CTTTTTTTGTCTCTCCTCCATTTTGGCATAGAGGATCATCGCTTTACAGGAAAAAATGGAATTTTGTCAA
GATTCACGCAATGGCAATGGTTCACTAGGATTTAACACTAATGACCCTTTGAACTGGGGC
Sequence ID: 87
Sequence Length: 3726
Sequence Type: DNA
Organism: Poplar sp.
CCCTTAGAGTTTTAGTTTTTTTTTTTGCAATTGTAATTGTAACTAGATATTACAAAAGTTTAGGTTATAT
AGCATGCGTTTTTATGTGGTCAAGTTAAACCAGTAAAGAGATCAAAGCAATCTAGTATGCGTGTGCACAC
ATATATATAGGATTATAGTTTAAGCATAAAAAATCCTAGTACGCACTATCACATACAAAGCAAAACTAAT
TAATGATAAAATAGTACATGTTTAAGTTGTTGACATTTATTGGAGAATCGACAATTCATTTTGTCTTGTA
TTGGAAAATTCTTGGATAAGTTTAAGGTACTTACTTACTTAAGATCATAGAACCAAAAACAAGTAATAAT
TTGATGATGTTAATGATAAATCATTTGTCCTCAAGCTTTTATTGCTCTTCACATGATGTGAACATGATAA
TTATGTAATTATCCTCCCATATTCCAAGTACTCCTTCTTATTTAAATGTATTATTAAACAAGGTTTAGGA
ACTAGAGAAATTTAAAACTCAAAATCCCTAAATAACAAGCACTAGATTAGGGAATGAAAAAAGTTAAAGA
CATGGCAAAAGGAATGGTAAAATAAGCTAAAAAAAACCACATGGAAAAAGGTTAAAGAGCTACAATTTAG
TGTGATGAACATCTTCCATTTAACCCGTATCTATACTAATTTTTTGAACCTACATTGGTGAAGGATTAAT
CATGAGAAATAACCATAATTAATTCTCATCTTTTGACCTTAAATAACCCTTTAAAAAGGAATAAAATTTT
GATATTTTTTAGCTTTAAAACCATATGTATTTCCTTTGAAAACTATTCAGGAAGATGAAAAAGATTAAAT
GTTCAGACTCTTTATTCATGGGTTGGATATTCATAAATGGACCAACAAAAAAATAATTCTATATGAGTAG
AGAATTATTTATAAAAAACTCAAATCCAAGTCCAAAATAAATTAGCAAAAAACATAATCAAACCGACACT
TGAGGCCTATGCCCGAGCCGAGCCAAGTACGTGCGCGTTAAACTCAAACTATTTTGCTAAGACTTATTTA
TGACTCTTCAACTTTCCATTATATAAACTACAAGAAAATCTTATTTTTTTTTCTATGTGGGATACAAAGT
ATTGAACTTTATTTCATAAAAACATGTTAACTAAAGGTTCTTTGAGATTAATTTTTTATTTATCTATAAT
TTATTTTAATTATGTTAATATTCCATGACAATGAACTCCTATTAATGAATAATTTCCAATGGAATTTTAA
CCCTAAAAAAAAAAAGAATTCCACTTTAAACTTGACCTAATATATCTTTTATTAAAATAAATTATAATAG
TTTAGAGATTTTTTATGTGTCTTAGTGTTCAAGAGATAAAAAAAGAAGGATGAAAGTTGGAGGGGTAATA
AAAAAAAACAAAAACAAAAACACGATGAAGATTGGGCAAATGACTCAATTTATTCTTGCATAATTGAACA
TTTTATATGTACTTACATGTTTAATTATTATATATATTTTTTATAATATACATCCACATAAATTATTTTT
TAATTTTTCGATGTGAAATAACTATATATAGCCTATATTCACATGGATTATTTCTTAACTTTTCAATGTA
GGGTAACTATATAACACTTTATATTTTTACGTTTCGATGTGAAATAATTATACTATACTTCATGCCTCGT
ATTTTTTTTAATAACCTACATCCACTTACATTGTTTCTTAATTTTTCGATATGAGATAACTATATATGGC
TACATTAACATGCATTATTTTTTAATTTTTTAATATGGGGTAACTATATATAGCAATATCCATATAAATT
GTTTTTAAAATTTTTGATATGAAATAAATATGCTACACTTCACATTTCTTTTTTATAGTCTACATTCACG
TAGATTGTTTGTTAACTTTCCAATGTGAGATAACTATACTATACTTTTTACCTCATATTTGTTTTTTATA
GTTCACATTTACATGCATTGTTTCATAATTTTTCTATGTGAGATATTAAAATTTCAAACATATTTTTTAA
AAACTTTCCGGGTTTACTTGAGTCAACCTGTGACCCACGACTCGGCTCCTTGCTTGGTCAATCTCCAGGC
CGCGTTTAATAACTATGCTTATAACTAGTGTTTTTATTACTTAAGTTTGTGGAACACAAAGTTAAAGAAC
ATTTGTTAACGATCTCGTTATCAAACACATCCTTAAATTTGTAATACTTAAGACACCATTTATTTTCTTG
TTTGGAGCTGTGTTTGCGTATATTTTAAAGGTATGTTTGGTTTGAAAAATTATTAAATTGATATTCTTAG
TATTTTCTATAATCTTGATATGTTTATATATAAAATAAAAAAATAAAAAATAAAAAGTATTTTATTATAT
TTTCAAATAATAATTACTTTAAAAAATTATGGTACACTACAATAACACACAACCAATTAGTTTTGGCCCA
TTGATCTTTTAGAACATAAATGTAATTTCACAACTCCTATGCTCCGTTTGTTTACTCGCAATAAGATTTT
TATGAGGCTCCGTGAATTTTAATTGTTTATTAATCAAAATAAGATTGGAAGCATACACTTACTAAGAAAT
GTACTTACATAATTTAAATGAGTAGACAGTATAATCAAATTATAAATTTCAATTCATTTCTAAACATTCC
AAACTACATGATTCGATTTTAATATTAGCATATTAAAATAATAATAAAATATATAAAAAATAATTTTAAA
TAAAAACAAATATTAAATTTTTGGCGACCTTTATTAAGATCGAGTTCCATGTTACAAAATTCACGATACA
TAAAAGTACAGAAAACAAAAGTGTCACTTCCCTCGGCCAATAGAACTGTAGAATCAAGCCTTCGCGATCC
TCGTCACTTCTCTAAGACCACATCCTTGCCGTTCATATCAAGTAGAGAAAACACAAATTAACAATGTTTA
AACCGTCCGATAACAAGGAGAGAAAGCACAAAGATATCCAATCTTACCCGTCCGATTCCTAACTCCAAAT
CTCTATAAGTAAGCCTTGACTTCACTAGATTTCAAACTTTACAAAAATTCGACGCAGGGAGGGAGCGAGA
GATTCTCGAAGAGATTCAAAGTTGCCTTTTTCGGAGTATTCTATTTTCTCTTGTTCCTAATTTAGGGTTT
CTGATTAAGTTCTTCGATCATTAGGGTTACTTGTTGTTTGAAATTAAAGTCTGCTTTGGGGGATTTTGGG
TATTGAATTTGTGTGTTCTTGATTCGGTGTTTGAAATTGTTGGATCCTTTTCGAAGTTTACGGTTTATTT
ATCATTTTCGTTTCAATTAATTAATTTTTCTTGCGTTGTTATGATTGCAATTATTGATATCAACTTTCAA
ACCAATTTAGGATTTTTTAGACTTATAAGTTTTGATGGAACTGTTTTTTTATGTCACTAGTTGTGTAATA
GTTTCAAGGTCATTCATATTTCGATAACATTTGTTGAATTGTGGTTATGTTTGCTTGCTGAGAAAATGTA
AGGAAAACACATTTTCGTTCATGGCCTATGTTATAAGCTTGATCAAAGATATGAATTCTGCATAAGACGT
GTGGTTTTGATTATGTTTTCTTCTGTTTCTCAAATGTTGCCCAGGAGGCCTAAAGCGAGGTGGTCAATAT
CTGCATGAAGTTGTGTTAATGAATGTTAAATGTTGTCTTTTATTTGTTATTTGGATGATTGTATCTGATG
AATTGGTTGTGTATAG
Sequence ID: 88
Sequence Length: 3741
Sequence Type: DNA
Organism: Poplar sp.
CTGGGAAATTATTCAATATTCTTTATACTATTTGTTGGTTCTTCACTTGTGGCTGAAATGCTATCTTACA
ATAATAGGCTTTCAATGAAGATGCAGCACCACCCCTTTTTTCAATGTTCCTTTCTATGAAACGTTAAAAC
TTATTAGTAATTGTTTTTAGTCTATGTACTGTTGGATTTTAGCTAATCTACCAACATATGTAAACGGAAT
GTGTCTTCAGATTCATATTCTGCAGATCTGTGCTCTCTGTCTTCTGCCCTCTTGAAGATGAGCAATATCT
ACCCATTTGCCTGCCCCATCTTCCCAATTCTGTCTCCGCGAGATCTGAAGTTGTGCAGTCAGCTGTATTT
CGGCTTGCAAATCACCTGAAGGTTGCAGATTGTTTTCAATTTGATGACAGATAAGGAAGATAAAACTATA
AAAATGCAGGTTTCTGGCTCTCGAGTTCCATTAACGTTATGTGGTAAATAACTCCTGGAACTCGATCGGT
TGGTTTAAGGGCACTTCAAAGGAGTATTATAATTTGGTGATAGAGCTTAAGCTGGTGGTCAAGAATAGAA
TATTGGCTGAGCATGGTTGCTTCTGCTGACATGCGAACTCGTTAATTCTTTGGTCAGCATGTTGTGGTTT
GCTGTTCTAGGTGGTACTTGATGATTTGCAGTTGTGTATGAATATAGCTGCTTAGTTGTCTAGGCGTTAT
CAGTGTATAATCTAATTGGTTTTACACCTTCTTAGAGTTGTTTTCAAGCTAGTGCTAAGTTTTGTACCTT
TGTAAAAGAAAATCAGGTTAGTCGAATGGGATATATGTTTTCATTTTCATTTTTTTTTAAAAAAAAGTTA
TAGTGGCAACGGATTCATACATTATTGTTGAAGCTGTGGGAGAGGCATATATGTAAATGGGTCCTGAAAT
TGAAATTTCTGGATCCTTTCAGCTGTACAATTTACAAATTTGACTCGGCTGCTTTTGTGTTCTTTGGTGT
TGTAGCTCCAGTTGCGCGCTTCATGTGATGATTAGCGTCTTTATTTTTTTACTTTTTTTTTCATATTATT
TAAATATGAAAAAAATTATAATCAAAAGTTATCTTGGATAAAAAAATTATATCTATTTGAAAAAACATAG
AAGCAAGAAAGAAAAAGAAAAAGATCTTTCTCAACGCAAGAAAGGATTGGTTTTGAAGGAAGGAATTAAT
TAATTATTTTTTGTTAATTCACTTCCATATTTTATATGATAATTCTATCTTTTAAATAAATCTCACTTAT
TGTAATTCATTCTTGTATTGTTGTAATTTTAAGTGAGTGGAAGCATACACGAATCTAAATCTAAAGACAA
CTCATTTATATGAGTTGAATTTATATTTAAAATTACAATTATGAGTCTACTCTTTATGATTTTCACTTAT
TATTCAAAAGATTTGACTTAGCCATTAGCTTTGATACATAAAACCACATAACATATATATATATATATAT
ATATAAAAGATGTTTGGGCCAAGGCCCAAGAAATCTTGATTGTCCTGTCCAGGTCCAGGGGGAAGGCCTC
CAAGTTGCCCTGGCAACGACCAATAAATCTCCCTTTTCTCTCTATATAGGTTTGCTTATCTCGTTAACAT
AAAAACACTAGGAGAAAAAGTTACAGGCCGTGATCTCATTCTCTGCGCATGATGCGCTATTACAAGGGCT
GAAACATAGCGGACCCATTTATGTCAAAATTGGGGAACTACGAGCGAGAAGGGCATTAGTGGACACCGGC
TCTTCTATCAATATCAGCCTTTATCTTGCATAAGTTGGGAATAGAAAGGTGCCGATCTCAACCTTCCTCA
TCAACCTCCAGCAACTTGCTCAACATGTGAGCCATAACCTTTGCTAACGACCATGTTCTTCATCAGAATC
CCATTCCAGTATTCCAAAACCAAAAAAGCCATGCAGGGACAAGGACAAGAATGTTGCACACTAGATAGAA
GATTCTACACAGTTTTGCTGACTTGTGACATAAATTTATTGCTCGCTTTAGCACCGATATGCCAAAAAAA
AAAACACTCCATGATACTGATTTTTTTTTTTATTATTTTCATCCTTTATATTGATTTAATTTGAAATTTG
AATTTTTTTTTATTTTTTATAAGATTATTCTATTCTCAAAATTCATATTATAGGTTAACCCAAATTAACT
TAATACTATTTTGTAAATTCCTTTTCAAAATCAACTATTTTTTTAATTCCAATCATTTAATATTTTGTTA
CTTATCATTTAATTCTTTCATGTTAACTTGTAAGATTGTAAAAAACTTTTGATAAAATTGACAGGAATTT
TTTTAAAATATTGTCAGGAGTTCTTTTTATGCATGCTTTCGTTATCATTTTTTATTTAATTGATTTATTG
TGGTTGTTTTTTATTTTATTATATAATTAAATAAAGCTTGCTTATTGAACTAGTTTATATTATAATTTGA
GTCACGATTTTTTTTAATTTTTTTTAAAATATATTAATCCGGCTTTTGTATACCGAGGGCCAGAAGTCTA
ATAATAACAACTTATTCAAATGACAAGGAAAGAGAATTAAATATTAATTTTTTAATTAGAGCTTTATCAA
TATATAGTTTTTTTTTATAAATAAATATCCCTTGTGTGGATAACACATAGCATAAAGTACCCGATTTTGA
GAAATTACACATAAGAAAGTCCTTAATTGCAATTTCCTAAATCTGAATGGACATCTGACAAAAGACATGA
TACATAGTACCAACCACACATTGAACTTCCTAGCCACAAGGAAAGTACGGGCTTCGTCCTTCAGCCAATA
AGATTCGAGAATTAACCCTCAACGATCCGCGTCATTACTCTAAAACCACATCCTAGCCGGTCACATCAAG
TAGAGAAAACACAACTTAACATTGTTTGAACCGTTCGATTATAAAGAGAGAAAGCACAAAGATACCCAAT
CGTACCCATCCGATTCCTCCTCTAAATCTCTATAAATATAACTCGACTGCTTCTTTAATCAATCTTTACA
AAAATTTCAGAGTTTTAGAGAGAGGTTTCTTGAAGAGATTCCAAGGTGCGTTTTTTTGACTTCCTTCTTC
TGCTGTTCTTGATTATGGTTTCTAATTAAATTTCTTGATTGGTCATAAGGGCTTAGTGGTGTTTGTAATT
GAAAACTGTTTTGAGGGATTTTGGGATTTTTAGGGTTTGAATTTGTTGGTTCCTGAATCTGTTTCTGAAA
TTGATGGGTCTTGTTCGAAATTTAGGGTTTCTTTGTCAGTTTAGTTTCAATTAATTGTTATTCTATAATT
GATTTGATATAATTACGATTATTGATTTTAACTTTTCGGTAGTTGGTTTGAGATTCAGGGCTGTATTGGC
ATTTTAGTTCTAATGATGTGTTTCTTTGCGTCTTTAGTTGTTAAGATCTGTTGAACAGCGTTATGTTTGG
TTGCTGAGAAAATGCTAGGAAAGCCAATTTAGTTCTTGACCAAACGGATAAAGCCCTTTACAAGACTGTG
TTTAGATCTTGAATTGTTTTCTTGAAGACCAAAAGTGAGGCCTGAAGGTGCATGAAGTTGTGTTAATGAA
TGTTAACATGTTTGATCAAACAGATAAGCGCTTTAGTCTTAAGATTTTCAACTGTTTTCTTGGAGACCAA
AAGTGAGACTTGTGTTAATGAATATTAAAATGTTTTATCTATTTTGGCCTTCCTGATTGAATGATTGTTT
CTGATTATTTGTTTCGTGTGTGTGTGTATAG
Sequence ID: 89
Sequence Length: 3086
Sequence Type: DNA
Organism: Poplar sp.
ATGAGGTATGGTAACTATGTCATGGAATGTTTTTCATACTCCAAAACTAAAATGACATTGGCGCCGTGAG
ATATAATTTCATGTTGTCTAGAATAAGAAATAAATCAAATTACATGATACAAGGAAGTTAACAATCCCTT
CACATCCTTTATATATACCATATATATATATATTAGAAAAGCACCAAAACCACATGCACTAACTACCCTT
GCATCCTCATGGCACAGCCATTCTATTTAAAAAGAAAATACTAATCCATTTTCATCATCTCAGAAGTAGG
TAGCACATACCACAGAATTTCAGCAACTGAAAACTAATAATTAAGGTGATATTTCCATTTTTCTTCTTGA
TTAATGCATGCATCTGATCATTTGATATATCTTTTTCCCTAGACTATGCTAGCTATATATTTAACTTGCT
TGCTTAATCAGGCATGTATTTTTGCAGACCATGCAGGTGACGATTTCTTTTAATGATAAAGAGCTTGAAG
TGGAGGTTGAAGAGAATGCGACCGTTTTGGGCCTCAAGCATCGACTCCGTGATAAGCTTAGCCTCCAGCC
ATTGGCACAAGAGCTAGAGTTTGAAGGATTTATACTCCAGAATCATGGAAAAACATTGCGAAGTTACCAC
ATTGGAAATAATGCTAAAATCGTTCTTCGAGAGTTATTTGGTATTTCAATCCATGTGGTAAAGCAAGATA
AAGATGGAAAACCCGACCCACAATTTCCAATATCAGTCCATAAGGAAAATACCATTGGATTCTTGAAGGA
GATGCTTTCTATAAAGTATGGCGTGGAGTTTACACATATGAAACTAGGGCTCTCCAGCATGTGGAATAAT
AATCTTCATGATAGTACTAAAATTGGGGCTTATAATCTTCGTGATACAAGTATATTATACGCTTTTGAAA
TGTAATTAATTAATCACCTGTTGGGTGGTGAATTTATCTCTTGATCATAAAAGAAAAAATATTTAATAAA
TAGCATTTTTAAAATGTGTAAGGGTTATATTGAAATAAAATAAGATTTTCACTCTATTATTTTGGAAAAA
TGATATATTGTTCTAGCATAAACTTGAAAGTAATACATCATTTGTTTTTGTGTTCGAAATTGATATTTTA
AAAATGTGTTTTGTTTGAAAAGAGCAGCAAATTAATATTTTTTTAGTATTTTTTTTTATGATTTTAATAT
GCTAATTAAAAAAATAAAATAAAAATCTAACAAAAAATCATTTTGATATATTTTTAAGTTAAAAATATTT
TTTAAAAGTACCATGCATATTATAGGAAATTACTCTGAAACTTCCAAGACTTACACATATTTACACTTTT
TTTCCTGTAATTTTACAAATTATACTTTACATAGCATGTCAATGTAAGATTTAAATTTTGATGAAAAGAA
GAAAATACCCTCCTATATTATCAATCCATCTTTCTCTTCCTCTCCTCCCATCCCAATCTAACCAAAACTC
AAAAATTACACAGTACATACTAAGATTATATATTGTTTAATGTTAGGGCCGGCCGGATTGTTTATTGTCG
GGATCATTTGTTTAACTAGAAGATAGATGGGTGAGGGTGTGATGCATGGTGGTTGGACATTTAAATTTCT
TTGTGACTATCTACTTAATCGGCCAGCCATTAGACTATGTATTAAATCCTTGTTTTTGGATTTTATGTTA
TTGTTTTGTGATTTGCCTCTATTGAATTCCAGTTTAGAGACATGCTTAAAGACCTCTCTATTTACGACAA
AGGATCATAAATAGAAGAGAGTGTTGTGCTGTGTGTTTGTTAGGTTCATGTGGTTGGATTTTGGTGAGGA
GGAAAGGGTAAGGGTGGTGGCTTCTTTTTTTTTTTTTAATTCGATGATGATGAAGTTATAATATTTAATT
TATAAACACTCTTTAGTCATATAATTTTTTAAAACTTCATTGTTGACTGATATTTCACATACTCAGATCA
ATATGTAAACTATTATAATTTTTATTATTATTATTGTTAACGTGAGTGTCTGAGTCAGCTTGCACGCACT
TCGACTAATTCCACGGGCCCTGAAGTTAACGACCATGTAACTCTCTAGTGGCTATCATATTAGCAACCAC
AGGGCTCGAATCTGAGACTACATGGGAAGCAAACCTTTTAGTTTCAAGTTTCTATCACTGATCACCTACT
AGTTGTAACTAGTGGTATGTAAAATGTAATTTCTGAACCTACAAAAGGAAGATGTAATCGCTTATAAATT
CTGAGAGAGTAATTTCCATGTTTATCTCTATAAAACTGTCATATTTGCAACATCATCGCTTTATGATAAC
TTTCAATTCTTCACGTTAAATTTTATGATGGAGGCAAATGGATTATTGAAGCTTATTTTTGTTCCTTTTA
ATTTTACTATGTCCAAGCCCACGAAGAAAATTCATCTTTCTTTCTTTTTTGTTAGTATTTAAAAAATATA
TATATTTTTAAATGTACCAGATGTATTTTTAAAATAATTAAAAAAATATTTTTTTTCTTTTAATATTTCC
ATTGCTCCCACTTTCATTCCGATTTTTCCCAAGTGTTGCTAGGCCCTAGCACGAAGTATCAAGAAAAGGC
CCAATTACAACTAAGTAACAAAACAATTGCAAGCCCAACTAAGCCCAACCCATTAAATAACTTCCAGAAA
GTTCCAAAAAAACGCCTATATCTACCCATCACCGTCACTCCTTAACTCCGTTAACCATCCCACAACCTGT
ACCCCCACGTCCCCATCTCGCAAACGAGACCAAATACAGAACAGCCACCTAACACCGTAAAACAAAATCC
ATATCCCCGTCAAACAAAATCCATATCCTTGAGCAGGCAACTATCCTAACCTTACCGTGTCAACTAAGCA
ACCACAACCATACGACACGTAAAGTGCACACAGGTCAGAGTCTCATTGGTTCTCCACGTCACAGCCTCTC
CTTTGCCGTAACAGACTCGGCTTCCTCTCTATAAAAGTCGTTCCCCTTCCCCTTCCTCTCACACAAACGT
TACCATACATTTCTTTTCCTCAAGTTCTTAAAGCTCACTCTTTTCTGTCCGTCCGATCAAATCTAGAAAA
AAAAAG
Sequence ID: 90
Sequence Length: 3239
Sequence Type: DNA
Organism: Poplar sp.
TCGGTACTTCAAATTAAATGCCCTCACCTTTTTATGTCATAACTATGTCAATAAAAGGACATTAGAAATA
CCCATACCTAGTGGATGACTCATATGATAATATTTTTTTCAATCAATATTGAACCACTAGTTATTAGAAT
TGTTACCTCAAATGTTTGCCATTAAATTATGTAATACATATCTTTTCCATTCTATACTTATTTTTATACA
AGTTAGCATGATTTATGACTATATAAACATCTATGAACCACTGAGTAAAAAAAATGTCCTTATTTCCAGA
TTTATCAATGCATATTGTTATCATCTTCTTAATTATATTTTTGCAAACATATACATCACATAATTTATAA
GACTTCATTTCTTTTACATTTATGTTAACTCAAGCATCAGATGGTCTTTTAAACCCAACATGGAAATTGT
TTATTAGGTACTCATGATTTTCAAGTCTAGTAATTTAAATTCAATAGTTCAACGCATTTGTCAGCCTTGA
AATGTGCTTAATCTTCTGGGAATTTTCCTAAACCTTATCATTGGCATTATATGTGGGTAATTTGAACAAA
AATAATTTCATTTTCCTTTATTTTAACATATCGTTCATTTCTTTCCATGACTAGCAACTCAAAAACTTTG
GGACATGAGATGGTCATATACCTTCCCACCACAAGTACTTCACCAGCAAATAAAAAGCTTATAGAGTCTA
TTTTTGTCCATACAAAAGAAATTAAAGGCCTTACCTCTTTCATGGTAGATGGCTTCTCCATTTATTGGAC
AACTAACACCTTCTAATATGTGAAAAAAACTTTAGCTTAATGAGGGAATAGGCCATAATATGATATAAAA
AAGTAGGAGAGAGCATCATCCATCCCACAACCCTCATAAAAAGCCATTCTTAACATCATAAAGAGAGTTC
CTCAAGTGGATGCTCCAAAAATGGCAAAAGAGAGTCAAAGGATAAATTAATATATAGTCGCAATTATAGG
AGCAACCACCGCCATCATCACCACTCTTCATTAGTGCTTTCCTCAGGTAGCAGACAAGAAAAAAGTCTAG
AGCAAAGTCAAGTTTGTGGTGATACGAGCCTTATACATATTAGTCTATATCCCTACCAAGTGAGAGGTTC
TCAATTAGCCAAACATGTATTAGACACTCGTCTCTCGAGATATTTTTATCTCTATAAAAATAAACCTAAA
CAGCCATGATGGGCTCATAAACCGATGAAAAGTGTACTAAATATGAATAACAATCTATATTTGTTACATA
GGATTCTGATGCGTTATGCATGATTCTCCCTGCCATTTTTCAAGGGCCAGCCTGCATATGGTATCTTAAT
TTGGATCTTATATCCATCCTCAACTGTGAGAATCTTTGTGCAAAGCTCATTGCCTAACATAAGTATCCTA
AAAGACGAGTGTAGAAAGATAGTCTCACCCACGCTTTCAGAAGTGAAAGGAGTCCTATAAATATGGTTGG
TCTTTAAATAGTTTAGGGCTATTGAGATGAATCCAAATGGGTTAGTTTATAATTGCCATGTCTTTCCTAG
ACTTTTGACCACGCTTCAAAAAATAACCTAGATAGAGGTTTATACTACCATCAAGGATAATGACTTCATA
CAATATCCCCTCCTACGAAGGGTGATCTTTATACCCATAACCAAAAAAATATTAATAATATAGAGAAATG
TTATAAAATAAAAAAGTTTATTAACCAGAGCTATCTTCAATAATTCATGAAAAAAGATGCCTAGCCTAAA
ATAGAGGAAGAAAGAGACTGACATGTATCACAAGGCATTATTCAAGATCAATTTAATATGAGAATTGTTC
TGCATTGAAAAAGGAAATATAGAGGTTCATCAGCAAAGGTTATCTTCAATAATTTAAAAAAAAGTTGCCC
AACTTTAATGGAATGATGAAAAGGTTGGCGCACATCATGATGAATTGTCCTAAATCAATGTGATATTAGA
TGAATTGTCAGTAAGAAAAGACTCCATGAAGAGAGAAGAGCTTATGAGAAGTAGGTTATCGTTATCGCTT
GTACCTTGAACCCATGATACAAATCAACTATTTTTACTATAAAGAATGGAGAAGGGGTAACTTTTCCACA
TAAAGATGCATTAGTGATCAGTATCATCCTCTTCCACCATTCTTCCAAAGAAAGTGATGAGTAAGATGAA
TAACAAACTTTCAACATAAAAAAGAAATAAATATTTAAAAGTATGGTATAAGAGAAAATAGAAATAAAGA
ATAATATAAAAGCTTGCAAATTGATTCTACATCAATATTTCAAATTAATGATCAAACAAAATTATTTAAA
TTGGTTCGAAAATTGTAAAAAAAAAAATATTAATAGAATTTTTATCTATAGATTAGAGGTGTAGATAAAA
CTATAATATTTGGATTATCTTTTTAAAAGATTTTGATTTTTAAGTGAAATAATAAAAATTTGAATATCCA
ATTGATGTTTTTATCAAGATTTTCTACTATCACGATGGATTGTTCTTTCAAATACAGTCAAGATTTAATT
AATTTGATTATTTTCATCAAATCTTATAATTTTATTCTTTGCTGTATCTTAATAATAAATAGAGTTTTAC
CATTATCTTTATAATTTATTTCTCCTTTATCCATCATCATTGATCACAAATAAGTATTCATTATTTACTT
TTTTTTTTTATAAAAAATATCATCATCATTAATAATTTAATCTTTAATTATTTATAAATTACAACTTTAA
CAACTCATATTTTCTTTCATTTCATCGCAAAACCGTATTCTAATCTTAAATTTGATAGATAAATCAGTCT
AGTCCATCCACATTGAAAAATAAAAAGAAAGAAAAAAAAATAGTGTAATTTGATAAAATTGCAAGGCAAA
ATAAAAAAAAAGAAATAAGTGCAAAATAAGGTTAAAGTTTAGGAGGTGTTTTGTAAAAAATGTGTTGAGA
ATGATTAAATACGGAAAGAAGTCTCTTCTTATATTGGACGCGCTTAGTAAGACCTCTTTTGCGAAACACG
CAAGAACAAGCCCCACCGCACCCCACCCCACCCCACCCCACCCCACCAATAAAAAAGGAAAAGAAAAAAG
AAGCCAAACCCTCCTCCTCCTCCTCCTCCTCCTCCTCTATATCTCTCTCATGGATCTTTGTTCTTTGATA
GATTTACTCACATAATTATAATCGTGAGATCTCTCTCTCTAGAAAGGTAAAGGAAGAGGAAAATAGAAAC
AACAAGAAGAAACAGAACG
Sequence ID: 91
Sequence Length: 3727
Sequence Type: DNA
Organism: Poplar sp.
GTAATGTTTATAAGTAGTTTCTATTGAATAATTCATTCATTCATATGTTAAAAAATTGTATTTGATATAT
ATATTTCTTTGATTTTTTAGGAATATTTTAATTTTGAAATCGTTGTTCAATTGAAGCTATTTGATATTCT
TCCTTATTGATATCTTGGATAACATCTTGATTTTTAAAGGAATCAAGTGAAAACTTTTTAAAAAATTACT
CTTCAATAAAAATTCTTCAACTTGGAGCATTAATACATTTAAAATAGATTATAATTGAAAAAAAATTATA
TTAAAAATCTCTTAGTTCACATGTATTTACAAATCCAAAACACACAATCTCACATCAAAAAGTTATAAGA
TTTTGTTGTAGCTTATATAATGAAAAATTAAAGATTTAAAATTTAATTCATACACCAAAATTTTTAGAGA
GAATATGCGCAACGCTAAGTGATAAGTTGTGGGCTTTAGTCACAAAGACTTGCTAGGCTTGGCGCCGAAG
TCTAGGCTGGATAAAATAATTTGTTAGTCTAAAAGAATTATTATGTGTCCACCCATGATTAAATTGTGTA
ACCATTCTTTACCTAAACAATTTATTTTTAATGGGAAAACATCTTATTTTAAGGCAAAATTTTTTTGTCA
AAATCTTTGTTTGTGGTGGGTAATTCAAGCAATACAATGAGAATTAATTATAATAATTACCAAAATTATT
TTAACATCAATTTTAGTTTCAAAAATCATTGTAAAAAGGGGTAAAGGGGAAGTGTTCTGTACACCATGCA
GTAGCTCTTACACTCTTCTTTGTTATTTTCCAATCTTTTCTACTCAATTTTTCTTCAATCATTCTAGAGT
TCAGATTTAGATTTTTTTAAAGAAATTTTCAGCCTATTTTTCTAAGTTTGTAGACCTTAATTAATAGTAA
AACTAATTAAGATAATGGTGTTGGATTTTTAAGAGGTTAAATGCATTCAATCTATTTACACCAAGTGATG
AATAATAAGAATTCAATGACAAATATACTTTTTACATCTTTCATATCATCTAAATGCTTCAAAAAGTCAG
TATATACATAACATTCTGTTTGAGATATTACATATACAATTCTTTAAGAACATCAACCACCAATTTGTAA
TTTATCTTTAAATTATTTTATACTTACTCTTCAAAACTAACAAACTTAGGGTTCCTTACTCTTCATCTGT
ATGCAAAATTCATCTAGTTAAACCAAACATGTTAAAAGATTTAAAACAAATTCCTAGTCATTTAAATCTA
TCGCGCACACGAGGACCCCGTTTATTTACTGGAAAGTAGTTTCTTTTTGGAAAGTGAATTCCGGGAAAGT
GAATTATTTTTCGATGTTTGGTAGTGTAATAGGAAATAAGTTGGAAAACATCTTCCAGTGTTTGGTTATG
TCATGGAAAATGAGCTGGAAAATAACTTATTAATTTTTTATTTTTTTTCAAATTTATTAAAATAATGAGG
AACAAATCTTATAAATTAAAAAGTTGAATGTGAATGAAATTGAAAAAAAATATAATTTCATAAATTATCT
CAAATAAAATAAATAATAATCAAAATAATAGAGATCAAATCTAACAAATAAAAAAATTAAAAGATGAAGA
AATTAAAATAATAATAATTACCATTTCATAAATTATTTCAAATAAAATAAGTAACAATCAAAAGAATGAG
GACCAAATTTGATAGATAAAAAATTTCAATTAAAAAATGATAAAGGAAAAGCAAATAACAATTATAAAAA
TGAGGACCAAAGTTAATATAAAAATCAAATTCTAAGGGATGAAATTAAAAGAAATATATTCAAAACAATA
TATATATATATATATATATATATATATATATATATATATATATCAATTAAAAATTTGAGGACCAAATTTG
ATATAATCAACAAATAATATGATATTTTTAAAATTTTCACAACTTTCGAAAAATATTTTTCGCTTAAAAT
AAAAGGAAAACACTTTCCTAGAAACCAAGTCGAATTTTTCTTTAACTAAAAAATGTTTTCCGTCGATCAG
CTAAACACAGAAAAGTTTGAAAAATAATTTTCAAAAAAACATTTTTTTGTCAAACATACAATGTCTAGGT
TTGCAAATATTGAAAAAAGTCAAATCTCACGTCTTTAAGCTTTTTTTTTTAATATAAAAAAATTATTATT
TTTTATGATTTTATAATTTTTTTTTGTTTTTTATTTTTTTTCATCATAAATGGATGGTGACGGAGTTGTC
CACCAAATCCAGACCCCTTTTGCAAGTTGTGTGATCCATATTATAGGCGTAAGCAGACGGCTACCCACCG
TTTACTCGTTCCTTTCCCTTCCAACTCCGCAACACTCCAAATAAGAAAGATATCAACTAATCATTCAAGG
TGCGTGGACTACACTGCCTCAATTACTTAATTTTGGTTTTTTATATGCACACACTTTTAACGTGGAAAAT
ATTCTATTTTGGCTTTCTTTTTATATTTTTATTTTGTATTTTTATTTATTTATTTATATAAAAGATATTT
AAAATAACCTTGCTAACTTGTGTTTAAATTAAATTATTCTATCTAAATATTTAAATCAAACCTCAAAATT
ATACAAATTAATATATATTTGTAATTTTATTTAGTTTGATGACTGGAAATCAACTAGTAATAAGCTTGTA
GAAAGAAAAAAATTTAAATTTAACAGTAGATAAAAACAAAATAAGATTCTAGCCGTAATAAATTTTTTAA
ATAAAAAATTATTTTCAAATTAAAAATTAACTCATTATTTTTTCATAGCAATATAACGACGTAAATCCAA
CAGAAAATTTTTTTTTTTTTCCTTACAAACAAACAGGAAAATTCAAATACGGAAATACCGCATAACACAC
GCGTACTCGAAGAGCGTAGTCCTTTTCTCTCACTCTCAGTTTCCTAGCAATATCCGCCCCCACTAGACGC
CACTGATAAAAGGAAAAACAACCAAGAAGTCTTCCGGAAGGATCATCCTAAGACGTGGCGTTTCATAATT
AGACGCTGCAACCGGGTATTCCCGTAATTTCACCATCAAATTTCTATGATATATTGAGACGCTCCAGCTC
TCTTCTCCTCACAGTTGTGATTATTTCTTCTCTATCAAAATCTTTAAACAGCTCTCATTCAAGGTATGCC
TCTCGTGTCCGATTGTCTAGTTTTTTCTTAAATTTATTTTTAAATAAATAATTGTTATACGATCTGTTAG
TGTTTTATGGATCAGTCCTTAATAATCGTTTATTGATGACAGTAATCTGTTTTTTTTTTTATCTGGATAT
TACGACGTTGTTATTGATTCTATTGTTTAGATCTCGATTGATGGGGGTAATCGAGGACCCTTTCCATTTT
TTTTTCAAATTTTGATCAATTGATTTATTCTGTTAAGATCTGTTAGTGCTTTACGGATAACATAGATCTG
CCTGATGTTCAGATTTATTTATCTCGATCTTAGGGCTCTATTATTAACTCTGTTTAGATCTCGATTGGTC
GGTGTTTTCAAGGGCCTCTTAAGATTTGATTAATTAATTAATTAATTAATATGGATCATGTTTCTATTCG
TTTAATTTTGATGCTATTTTGATTAAAAGAAAAGGTTATCGTTTTTTTTTTTAAATGATCAATTGAATTT
GTTTGCTATTGTTAATGATCTGAAGGATTGTTATATGTTGAGATCTGAATCTAGTTTGTCTCTGATTTTA
CTTTTTTGGGGTGTCAG
Sequence ID: 92
Sequence Length: 3639
Sequence Type: DNA
Organism: Poplar sp.
AAAAATGTGGCCATATGGCTGGTAAGGTGCTTGTTGCTATTAGTGAGCACATTAATAGGCTTGTAGCGGC
AAGGCTGCAATTTGATGTCATGGGGGTGGAGACGGTGCTGGTAGCTAGGACTGATGCAGAAGCAGCAAGT
TTGATCCAAAGCAATGTGGACGCTAGAGATCACCAGTTCATTTTGGGTGCGACTAATCCAAATCTTAGAG
GGAAAAATTTGGCCAATCTTTTGGCAGAAGCAATGGCTGCTGGTAAAACTGGAGCTGAACTTCAAGCCAT
TGAGGACAACTGGCTTGCAATGGCACAACTTAAGACATTCTCTGAGTGCGTTATGGACACAATCAAGAGT
ATGAATATTGGAGAGTACGAGAAGAGGAAGAGATTGAATGAATGGACGAACCATTCTAGCTATGACAAGT
GCTTGTCCATTGAACAAGGCCGAGAGATTGCAGAAAGATTGGGTCTTAACAATCTTTTCTGGGACTGGGA
CTTGCCTCGGACCAGAGAAGGGTTCTATAGGTTTAGAGGGTCTGTGGATGCGGCTATTGTTCGTGGCAGG
GCTTTTGCTCCTCACGCTGATCTTATTTGGATGGAGACTGCTGGCCCAGATTTGGCTGAGTGCACCAAGT
TTGCAGAAGGTCTGAAATCAATGCACCCTGAGATCATGTTGGCCTATAATCTTTCCCCGTCTTTCAATTG
GGATGCATCCGGCATGACCGATGAACAAATGAGGGATTTCATTCCTAGGATTGCTAAGTTGGGGTTTTGC
TGGCAGTTCATAACATTGGCAGGTTTCCATGCTGATGCGCTGGTCACCGACACTTTTGCAAGAGATTATG
CAAAGAGGGGAATGCTGGCCTACGTAGAGAGGATCCAGAGGGAGGAAAGGAAAAATGGAGTTGACACCCT
TGCGCATCAGAAATGGTCTGGTGCAAATTACTACGATCGGTATCTGAAGACTGTCCAAGGAGGCATTTCT
TCCACTGCTGCCATGGGAAAAGGCAAGTCACACATATTTTTTGTACACTAAAGAAAGAATTGAAGTAGCA
AAAGCTTGACAATCTTGAATCCTAATTCTGTTTTGCAGGAGTGACTGAGGAGCAATTCAAAGAGACATGG
AACAGGCCAGGAGCTCTCGAAATGGGTGGTGCAGGATCTGAAGCGGTTGCTAAGTCAAGGATGTAAGACC
AGAGCTGCTTTTGAGTATGGTATAAGGATCACTTAGAACATCAACCAGTTCCTATATTGTGGAACTGGAA
GTGAATAATTTCTGGCAATTACGCCATTCTTGTCTTCTTCTTTTTACCAGCTACATTGTGCTCGTCATCA
CTTAGTCTAGCTCTGTCTAAAGCCATTGTCAGAACAGGAACTTGTTTATGGTTACTGTAATTCAGTTTCC
TTCATATGTTTGCAGTACTATATGATGTTTGTGCTCTACAGTACTTGCATTTCTTAGCAGCATACCTTTA
TCTTAGAATTTCATATTGAAACCTACTTTAAATCCAAAAAGAAAAGTATATAATTCAATTTCACCTCAGA
GACTCTCACAACTTTGAACATTTTTTTATTGGAAAGTTTCCATGACAATAAATATTTCAATAAATGTTTG
AAGAAAATATATTGATTGTCTTAATTAATATAAATCCTTGGAACTTGACATTATATGTGCGTGTGGAATA
TTTCTCTCCATACATAAGAAAGCAATAACAAAATTGATAATGATGAAGATAGTTCAATTAGTCAGCTTTT
AAGTTTGCTTTCTAGAATTGCTTTTTAGAAATCACCAGTTTGAGTCTCAAAAATCTCAGGGCCATTAGAG
CCTTACATGATCGTTAACTTCAGAGCCCATGGGATTAGTCGAGGTGCACGCAAGCTGACCCGAACATCCA
CGTTGATAAAAAAAAATTTGATAGTGAAGAAAACAAAAAACAAAAAGAGTAAACTCTTTGTTTTCAAGAA
GAAGATTTGAATTGTTTTGATGTGTTGATGTCAAAAATTATTTTTAAAAAAATAAAAAAAATATTATTTG
ATATATTTTCGAGTAAAATAACATTAAAAAACAATTATTAATACACTCACAAGTGTGTGTCTATATATAT
ATATATATATATATATATATATATATATATAAAGGTTTATGCAATGCTATCCTAGACCACAACACATCAG
GAAGGACCCACTCCATTTTCTTGAAGATAAGTATTCTTCTTCTTTAAGAAAATAAAGAAAGAAAGATAAG
GATGCCAATCATCAATGACGTCTATGTCCACGAAATTGGCTTCTCACCAACCGCTTTTTCCTCATGTCAC
GGATGACGTCGGACTAAATTCCTAATATATTAGTGCTAATATTTATTTTTAGAATAAAGTCCTTCCTTCC
CTTAAAATCAAAGTTTATATTCTTCTTAAAAAGCGACAAATTGATGCTTATAAAAATTAATAATCAACTA
TATTACATCATTATTCTAAAAAATTGTAAGAATTGTTGTAAGTTTATTGCTATTTTTTTCTTTTTTTTTT
AAAATAAAGATATTTTCAAATTAAATTGAGATTTAATTTTATTTTTTCATAATTTTAAGGATATGTTTTT
ATATTAATTTGATGCGTTTAAAAAATTAAATAAATGACAATGTTCAAATGAATTGAATTTTTACTATTTT
AATTACTTTAATAATAATACTTTGGTTTGTTCATGTGCACATTATACGGGTTTTCTATTTTTTAAATATA
TTAAAATAATTTTAATTTATATTTTTTTAATATTAACTTATTAAAATCATAAGAAAATATTAATATAATA
TTTAAAATATTACCAATTTCCACATAAATTAGGCACCACTAGTTTTTTTCCTTAAAAAGATTCTCTGTGT
CGGTAGGATAAGATGAGCGCGTTCAAACACACACGCCAGAAAAGAGTGCTTCACCTCTCTTTTTCTCCAG
GGTATTCTCGTAACTTCCTCTATAATTTCTACTATAAAGTAGAACCTCGAGCCCCGAGTTCCTCACAATT
CCGATAATAGCAATCAGCCAATACAAAAAAATCCCTAAAAAATCTCTTTCAATTCTCAATAACTCAATGT
AAGTTTCGTTCCTAAATTTCTCTTGAATATTGGTTGACTGCTTAATCTTTGGTGTGATTGATTGATTGAA
TTGTGTTTTCGATTTGATTAGAAATCGTTTTCTTTTTGTTGATTTTGATCGATTTTCAAAGGATCTGTCT
TTTCTTTAGAATTTATGTAAATATTTGGGATTTGTTTCTTTATGGACATGATGATTGCTTTCAATTTGGT
TAAAAACAAGATGACAGATAATTTTGTAGGTTATATATTGTTCCTTGATTGAAAAAAATTTAATTATTAT
TTGGAAGGCATGATTTTTTAATGGTTCTTATGATTTTGTGGGTTGGGCGTAGGCTTCCGTTTTGATTGAT
TTCTATTTTGAGGGTAGCTGAATTGATTGATATGTGAAAGATTTTGTTAAAGAGTCTGAATGTGTGTGGT
GGATGTGTTTATCTTTTTGATAATATTATAGGATCCATTGACGCATGATGAATTTGTGGGGCTATGAAAA
ATAGTTTTTTTTTTTTTCCTGTAATTAAAGTTGGCTGATCTTGTCTTTGTGTTTGTTATATGTGACCAG
Sequence ID: 93
Sequence Length: 3182
Sequence Type: DNA
Organism: Poplar sp.
CTGTGGCTCTGACTAAAGTTTCTTGAATGTAAAATAATAATTTCCAGATCATGAGTCATTAAACTCAGTT
TAATAGATTAATTTTAAAATCTCTTAGCATGTTTTTTTTCTAATTTAGATTTTAAACTAAATCATGGATG
TCCTGACATAACTCGATCAACTTGAAAAATTAAAAAACAACTTTAATGATTAATAAAAATATGATTTAGT
ACAATGTTTTTATCTAAATGTTTTAGTATATGTTATAATTATTTTTAAAACATGGTCCGACCCTATATAT
TAACTTAAAAAACTTGAGATTAATTCGGATGGTTAGAATAAGGTTTGGGAAGAATCCAAGTTCACCAAGG
AATATATAGAAAACCATAAATCAATGTAGCTAGTCATAATTGATTAGAAGAGTTAGAATAAGTAATCGAT
CAGGAAAAAAAAACAATTAACCATGTATATAAAATTTCACATAATCGAACAATGTGGTAAGTCCTATTTA
TCATAAAAGTTATTGATGGATTGAACTGATAAGTATTACAAATGATATGTCGATATATATATATATATAA
AACTAAAATTCAAGAATGTTGAGTCTATCTGCAAAGTTAGATTCAAGAGTTTTAGGTCTAGCTGTAATGT
CAGACCTAAAAATAATATTTATAATATTAATTATAATACCAATAGTAATATTTATAATACAAATAATAAT
ATTTTTAATAATAATAATAATAATAATAATAATAAACTTGTCTTACTCAAGTTTAAGTGAGTCTGGCTGC
AATGCCGTACCCAAAAACCTTGGATGTGGGTTTGGCTATAAGATTGTATCATGAAAGTGTGATAACTAAA
TAGAAAAAAATATTGCAAAAATAAAATTAAACAAAAAAAATCAACTGAAAAGAAAAGAAAAGGCAAAACA
CTATTCAAATGAATAGTGATTTGGGAGGAAGAGTAAAATAAAATCCCCTTATGAGATCATGATAATCTAA
TGAAAAATAAACAAAACAAATCATAAAGTTTAATTCTCAATCAAATCGATATTAAAAGATAAAATTAGAA
GGAAAAAAAAATTAAAAAAAAAATCTGAGTCAACTGCCTTAACCTACCAGACCAGGTTAACCCATCAAAT
CTGGGATCTGTGTCATGAAAATATGATAACTAAATAGAAAAAAAATAATATTAAAAAACTAAATTAAAAA
AAAAATAATTAAAAAATATTGTCTTTCATTATGGATTTGCACCGTAAAATCTACAGTGAAGGACTAATTC
TTTTTAGTTATAGTTAATTAGGTAAACTTCATATGTGATGTATATAAATAGTATAAAATTAGGATTAAAT
GTTTACATGAAACTCAAAATTTATAACAATATAAATAATTAATCTAATTACTGGGAATAATAATCAGCCC
ATATACACAATCTTTTTTATGCAATTTATCTTTTTTTTCCTTTATTATGTTTCTTTACTTTGTTTATTTT
ATTTTATCTTTTACTTCTTTTTTCCGTAACATATAATGGGACAATCTCATTTTATTTGTGATAGTTTTTA
AATCCGGCTCGGCAGGTTAACTTGAAACTCAACCAATCCATTCTCTAACTTAAGTCGGTTATAAAAAAAA
AAAAAAATCAATTTCAACTTGGAGGTGACCATGCAGGTCAACATGCAACATGATTGATCTATTCAAAATC
TGGTTTGATTTTAATTTTTTTTTATGCAAAACAACTCTGTTTTAGTTTTTTTTTTTAAAAAAAACAATGT
TGTTTTGATTGACATGTGTTAACTTGTTCAATATGTGGCCCGAGCCTTGGGTTGATTCATGAGCCGAGTC
AATTTTAATTGCAACACTACTATTTGTTTTTGCTTGTACTCGCAATTAAGTTTCTAATTGTAGTTTTTTC
TCATTGAAAAATGAAGTAGCATAATTGATCCATATAATTATCTTATTGATCAATGGTTTGACCTAGTTGG
GTTTCAATGATGCAGTCAAACTTGAATAGGTTAACATAAAAAAAATTCTTTTTAAAAAAAATACAAATTG
TTATCATTTTAAAAAGACAATTTTACTCTTTTTTTAATTTTTAATCCTTGATTTAAGAAAAATAAGAGAA
AGTTATTGAATTCTGATGGCAAAGAGATAAAATGATCTTTAACATCAATTTTAACCACCAAAAAAGTCAA
TTTAGGTATTAACAAGTTTCATTCGGTGAAAAAAGTTTCATTTATGTGTTTTTTCGCCTTAATTTTTTTT
TTATTTTAGGTTGATTTTAGGTTTTTATATGGTTGTTTTAGGCTATGAATGAGTTTATCAAGATGTTTAG
AGGGTGTCATGGGTGTTGTTCAAAATAGGTTGAAAAATGAATTTTTAGATAAAAAATACTTAGGACCCGA
TGAAAATTTTTATTTAATTAAATTAAAAGTCATTTTCCCTTTAATATTTAGAAAAGAGGGGTGGATGATG
CGTCTTTTGCCCATGTTATTGGAAAAAAAAACTCCGCTCATGAGCTAGACCTCTTTCATGGGCTTAGCGG
GTTAGGTTTTTTTATAGCCAGACATGTAAGTAGGCCCACATACCAATCATGTTTTTTTTTTTTAATTTAA
AATAATTCCAACCATTTAAAAAATTAAAAAAATATACATCATATCAACATTTCTTTGTTTTTAAAGGTCT
TTTTTATTTGATTTGAAGAAAAAAAACTCGTTTTGTTATAAAACTTGAAAAAAATTAAGACTATTTTTTT
TAGTTTCACACACTATTTTTTTTGTTATTTTAAATTGTTGTTTTATAGTAAAAAAAAGTCTCAGCTCTCA
ATTAATCTATAGTTAAATAAAAAATAATTTTAAAAATAAAAAAATTATTAAATCTAAAAAATGCACAACG
TAAATTGCGGGTTTATGAAGCTAATCTTAGCTAACTTAGTTTTTTTTTTTTAATTAAATGTTTTTTTTTA
ATTTTATTATTTAATTTCCTTTTGCATTTATTATAAACAAAAAGCGTTGAAATAGCCTACAACGAATAAA
ACTCTGGCCCACGGGGACAACTATCCTGATTCCTGCGTGCTCATACTTATCCCCAAAAATCGCAGACCTT
GTTTCGCTTTGTCTCGACTCTCGATCGTATAGTAGTGTTTTTCTTTTTCCGTTTTAGAGAAAATACAAGT
TTAGTAACACAGAAGAGAAGAAGAAGAAGACG
Sequence ID: 94
Sequence Length: 4014
Sequence Type: DNA
Organism: Poplar sp.
TTTTCGGATGTTGCTACTAGTATGCTGGTTAAAAGCGATGCCCTTCAACTGCTTGTACAATGATTAGCAT
CATCAAATAGCTTGCAATTGCTCACTCCGGTACCCTTCAATTTGAGTTTTTACAAAGCTTTTGTTGATTA
CTGCTGATATTTATTTATTTATTGGTATTTGGGTTTATTATATAAAGCTTAAATTGTGCTCTGGATGTTA
TCCTATACAGTTTGTTGTGGTCGTCCAAAATATTTATTTTGTAACTTTGAATATAATGTCATGAAGCTGT
GCATTATAATGTGAAGAAAATCTTGTGAAGTTGTACATTTACTTTTGCCATGTAGTTACTCTTCAGAAAG
ACTAGAGGAAGTTAAAAAACAAAAAAGAGCTCATCAAAAACTAAACCAAATCTAGGTCCTACTATTTTGT
CGTTTACCATGGGGAAATTCAATGTTTATGTTTATGCCAGACAGTTGATTTGGAACTTTACAAATTTGCA
TTTCCTTGATATTTCCATAATTTAATGGAATAAAAAGTAACCATGTCTTATGTGTTTATGTTCCTTAAAT
CAGTACAATAACGCTTTGCTAAATGGATATGGGATGCTTGCAAACATATTATTTCCTTATCTTTGGATTA
TTTGACAAGTAATTTGGTAAACCCCCAATCTAAAACCGGTAAATCAGTGTTAGAAACGGTAAATTTTCGA
TTGAGAAATGATAAATTAGACAGAAAAGTTTTGTGAATCAGAATGAACATTCCTAGATGATTAATTTAAA
TGTCGGATGAGATAAAAAAATTAATCGGTATGTGAAATATTAAATAGCAATAAATCATGTAAGTTTTTTG
ATCGGGTAAAATAATGATTTGAAAGAGGAAATCTTGTAAATTGTGAATGGCTTCTCCCATAAACCATTCA
TGGTCGACCACTTTGTTTAAGAACACTAGATAAAAGTGAGCCAGGAATTTGAACTAATAATTGAAGATTT
CTTAAAGTTTTGGAGAGCATAAAGTGATGGGTTTGGGGGAAAAAATTAGAGGAAGAAGAAAACTTGTTTT
TAACATCTTTTTCCTTCAAACTTCTTGTGTTTAAGAGGTGAGAAACTTTAATTCAAGCTTGGTTTGAGTT
TATCTTGTGAGTGAAATGATACATTTTAGTTAAAATATGCATGATGGAAATTTGAAATTGATTGAATGAT
TGTTTAAATTGATGAAATTGTGTTTGAAAAGATGGTAGAAGAAAAATTCATCTCCCACCATAAGATGAAT
TTCTGTCAGGGTGGGGGGGATCAAGGGGAGTTGGGAATTCGATGAAATTGTTATGAAGATTGCATAGAAT
TGTGGTGGTATAGGTAATTAATAGATAAACAAGATGTCAACATTATTAATTAAAAAGGGAAGGACCCATC
ATTTGTCTATCATGGAAATTGAACAAGTCTTGTTTATATGGAGGAGTTGTAATTGTCAAAATTGATGAAG
AATTTATGTTTGAATGTATGAAGTAAAACATTTAAAAAGTTGATGATAGATTAATTGTACAAGGAAATCC
ACCCTTATCGTAGACAAAGATCTCGACCAACTCTATTTAAAGGGTTTAAAATTGTTGGTTTAGTTGACTT
GAAACATTATGTAAAGCTGTGATGATGTTAGCATAAATATAGCTGACTTGAATGGGGAAAATTTCATAAA
AAAATATGTGGTTATGCATCAAGTTAATAACTGTAATCTTTTTATTGATTCGCTTTCAGCTGAAGTTTGC
ACTAATAAATAATAGAATTTAAAATATATTTTTTCATGAAAATTATAGTTAAAAATGTTAAATTTCAAAT
GGGATGGACCACGTTTAATTTGGAAAACTGAAGAAATGTAATGCTGGATTTTTTGCTGAATAGTTTTATC
AATTAACTTGTCGCTAAAATTTAAACCGGTAAATGATAGAATTTAAAATATATATATATTTTTTTATAAA
AGTTGTAGTTAAAAATGTTAAATTTCAAATGGCACTCCCCTCGTTTAATTTGAAATTTTAGAATTTTGGA
TATAAATTTAAAAAATATATATATATATTAAAATGACTTGTAGCATAACGAGGAAAATAATCTAGTTACC
CACTTAAAAAAATCAGATTTGCCGTAGTGTTGTTAAAGCTACAGGTTTGGCGTTCTATCATCTATCTTCC
CATAGTGCAAAGAAATATTCCCACAAGTGTTGTCTTGAATCATTACTGCGTGCGTAGATGTTTATAACAT
CGAATCGTGAGCAGGATTAGCTGTTGAAAGCCATTCTAGCAAGCGATGAGTCTAGCTGAGTTCTATTTGG
CATCGCAGTGAATTATACTTTTAAAATTTTTTAATTTTAAATTAATTTTTTATATATTTTTAGATTATTT
TGATGTTGTTATATAATAAATAAATAATATTTTAAAAAATAACATTTACAATACTCTTAAATATGCCTAA
CAAATCACAAACCCTACAGCAATCAGAACAAAAGCTATGAATTTTGATCCAAAGCACAAATCGGAAGACA
TGGCTAGAATGGACAGACAAGCAAATCAAATACTTTGATGTGAATCGTAGAAGCTATCTGCTGGGATGAC
TCAAGAAGATGAAAGCTAAAATTAAGCGCTAGCACCATCAGAATTGCATTAATTAGGGATGCAGTTACCA
TGATTATCATTAATGTAGGGCCAAATTGGGATTTGCGGGAAGATTACGTGTGAGTGAATGAAGGAGGGTA
AAAAAGGAACTTCACAGATGAAAAGGGCAACAAAAACATAATGAAACCGACATGACCTAAAAACACAAGC
GCATTCTATTACTGGCTGCTACGTGGTATCTTGGACCATTAGTTTAAAAACAATCCAACGGCCGATAGAG
AAACTTTATTCTATTACTGGCTGCTACGTGGTTATCTTGGACCATTAGTTTAAAAACAATCCAACGGCCG
ATAGAGAAACCCTAATCTAAGACCAAATGGCTTAGTGAGTGTCACTATTTAAACAAGCATAAGCGGCCAT
TCTCGATCTCTTTCGTTTGCTCTCTGTAACTGCGAGAGAGCTCCTTATTTTTTAGATTCCCTAAACTTCC
TTTCTTCAAGGTTAGATCTCGTCTCTCTCTCCCCCTTTAGTATTATGTTATGTTTACTTGTCTGATCTGA
TGCGATTCTCTTTTCTTTTATAGCTTTTAGCTTTTATAATTTTGATGGCATCTGTTATAAGCTTTTCTTC
TTATTTGTTTATATGCTACTGATCGTGTTTCATTTCAGATCTAATGACTCAGTTTTTGTTGATTTTGATT
GAAATCAGATAAGAAAAATAATTCAGACTCTGAATTAGTTTTTCTTTTCTTAAATTGTTTACTGTTCTGC
TATGAATATAATCGGGTTTTTTCTATCCTGAAAATCAGTTGCTTTTAGTGTTTTTATTTAAAGATCTTAA
TTTGAAACTATCTTTTTTTATTAATTTGTGTTTTCTTTTTTCCCATCGGAAACAAGGAAGAAGCAAACTG
AACCTTGATAACATTTGGTCATGTTTGTTTTGATGAAAGTGCCTGAATCTGATGGGAAAATGTTTTATTT
GACTCTGTAGTGACTGTACTTTAATGGTAAATTCCATGCCTTAGTTGCGTGGAGACCTGGGCTCGAATCC
CAGCAGTCACACCTCTGAAATTATTTTTTATTAACTAATAAGCGGATCTAGGCTTATTGTTGTATCAAAG
GTTTGCTGGTTTATTCAGACACATGTATAAGCTGTGATTGTTTTGCGGGTGGTTTTAACTTGTTATATAC
GAATGACTATTTGGCTTCTGTCTTTGGTTCATGAAATGCTATTTTTAAGAACGTGTCATATGTATGTCTT
TACTGTTGTTGAAATTATGGTCACTTGAAATTTAAGTATAAGACTGGTTAATTAGTATTATTGGAAATGT
TACTTTTGTATGTTTTTCTGTCCATTCAATATGTTTGTGTATTTATTAAGTCTTTTTTATTTTATCAATT
CCAGTTTCTCTCCCAAAGTTCATC
Sequence ID: 95
Sequence Length: 3000
Sequence Type: DNA
Organism: Poplar sp.
ACTTTGAGGGGAGAACTAGAGCACAATTAAATAGCTTTTAAATTTACAAATGTAACGTCAAGGTGGATTT
TTTTTTGTTGTTGTCTGTTTGAAAAGAAATATTATTTACTGTATATAGGTTGATTGATGATTTATTTGAT
TTGATTTGAAGCAAGTTTGATGATTTGATGTAGTTTTTTTTTAAAATAAAATGATATTAAAATAAAAAAT
TTTATTTTTTACATTAATATATTCTTAAAAAATCAATTTAATATTTTTTTTTTCAAATAGAAAAACAGTT
TAAATAGTATTTTAAAAAATAAATTAAATTACAAAAGCTACCTTGAATATTTCCTTATGATTCTCGGACA
TGGGATGTCAGGAATCCAGTATTTGTTCCCTTAAGCGCAAGCAGTATCGGATTCTAGAAGTGGTTGACTT
TCATGCTTCATAACAGCAAAACACTTTATCTTGTATAAAACTTTTTAACTCTAATTTGAGGATGAAAATA
CAAAGATAATAAAAATATATATGGTAGAAAAAATATAGATAAAAATATAAAAATAAATATATTTTTAAAA
TAAATTTATATTCTTTAGAGATATAATAAGAACATATCTCAAGATAAAATATATTTTATTTTTAAAATTT
TATTGTAAAAAACTCTCAATTCTAGATTTGTCCAACCAGGACATGCAATGATAAAAATAATTATCATCAT
AGTTTTAAAATCCGACTTGTGAGTCGATCTGGAGCAAAGTCCGAGTCACTAGTAGAGGTTCGGGTCACGA
GCTAGGTTGACCCGAGTCAACATAAAAATAAAAGTGATTATTATTATAGTTTTAAAACTCAATTTGAAGG
TCTACTCGGGATAAGGCCCAGGTCATGAGCAAGGTTGGTTGTTGACCCAGGTCAACGTCGGGATGAAAAT
GACTGTTATCACAATTTTTAAACTCGAATTAGAGGTCGACCCGAAGTAAGGCTCATGTCACATGTTAAGA
GGGCAAACCCAAGTTGACCCGAGTCAACATAAAATGAAAGTAGCTATTATTGTAACTTAAAAACTTGATT
TGGGGGTCAACCTGGAGCAAAAACCGAATCAGAATTAGGAGGGTCAACCCGTGTTGACTTGAGTCAATAT
AAAGATAAGAATTGTTATTACCATAGTTTTAAAACTCGACTTAGAAGTCAATCGGGTCACAGGCAGGGAG
GGTCAACACAGGTTAACATGAGTCGATGTATATATAAAAGTGATTATTTTATTTTTAAAACCCGACACAA
AGTTGACTTGGGACAAGGTTTAGGTCACGGGCGGGAGGGTCAACTCGACTGATCCATTTTTTTTTTTAAA
AAAAATTCAAAACAACCTTATTTTGACAAAAAAAAATTCAAAAAAGTCAACAGGTTTTTGACCCATGTTT
TATCCCGAATTGACTTGAGTTTTTTATCAGATCAGGTCGAGTTAATTCTTCCTTTATTTTTTTAAACTCG
AACCAAATTAGGTTCCGGATCAACCCGTAAAGTCAATATAAAATAGATTTTTTTATATATTTGTTTTATG
TATTATTCACTATAATCAAATATGATACAAAAAACAATTATTTTATCTTATATATCATTACCAACACAAT
TTTATTTTTATTTTATCTTCATTTTCTCAATCTTTTCTATTATAGAATAGTAACTAAATGACAGCTTCGT
TCCTGCACGAGACCAGGATAATATTTACTTATAATACTCCTAAGCCTGAGAGGAGGGACTTGACATGGAA
CTAAACAGTTTTCAAAACCACAAGTGCAGCATTAAAGTTGAATCCTGTACCTTTACTGTCTAGGATAATT
ACTTAACAAAAAAATAGTTTTGATGTAAAAAATATTTTTTAAAAAATAAAAAAAATATTATTTTAATATA
TTTTTAAATAAAAAACACTTTAAAAAATATTTATTATCAGAACTTATTTTTCTACAAGCATGTAATTTTT
TTTAGTTTATTCTCTTTATTATTACTATTAAAATAAAATCAGCAAACCCAAAAAACAAGGCATCTTCAAT
ACAATATTATCAATTATTATTTTGTTTTATTCCCAATTCCAAAAATGGGAAGGCTAGTTAAAAGCGTTCT
CGATATTTTGCCAAATTACCAATGCATGCGAGGACATGACGGTGATTATATTTCACAACGAGGTCATTGT
AGGAAACAGAAATATCACCAGGGTAATATAAATACGTCTACAAATCCTGTCAGTTTCCCTCTCTCTCGCT
CACTGCCTGTTCTTGTATTAGCCTGCTTCTCCTCTCTCGCGGCTAGGGTTTTAGCGCCTCTCTTCAGGTT
CGATTCTCGATGTTCTCCTGTTCTCTCACATGTCCTTGTTGATTAGCTCCTCTTATCGTAAACAAGAAAA
TGTTGTTTCTTTTTTCTCTGTTAAGCATCTGTGTTTTGAATCAGTTTTATCAGCCTTTAATTTTAGAAAA
GTCTTGGTTCTTGATTCTTCGTGCCTTTCTTGTTTTCTTTAGATTATTTACGGTTTTCTCAGTGAATGTT
ACTGGCATACATCGTCGTTGATTTTTATTTAGATCTAGTTTTTTCTGTGTTGGTTTTGATATATCTAAAA
GGATTCCTGTATTGATTCTATGATTTTCTTCAATTAGTTTAAGCACTGTCACTAAAATAAACTTATGATT
TTCTGTTTAACTGATTAATAATCTTAAATTGCTATACAAATGTGTATCTTCAAAATGATATGCTGCTGCT
AAATAGAAAGTATTTTTTATTTTTATTTTGGGTTTCAGTATTTTAAATATTAAGATATATTTGAAGTTGG
CTTAAACATTGTTTTTTCGATTGATTTGTGGTCCGAGTACTTGCATCTTACCCCTAGAGAATTTGTTATT
ATTCTGTATTTTACAAGTAGACCCAGTTGCATATTTGAGCTTACTTTCTCTACGTGGTTTCAGTGTCCCA
GTGTTTTGTTCTTAACTCTTATAACCTTTATATATGACAGCTCAACTCTATTACATAAGT
Sequence ID: 96
Sequence Length: 3847
Sequence Type: DNA
Organism: Poplar sp.
TAAAAAAAAACACATAAAACATCAGCTTAAAAAAGAAAAAGGAGGGAACTAAGCAAACCTAGGTGAATCT
CTTAAATCTAGTCTAATCTAAAAAACTTGTAACTTGTTAAATCATGGATCCAAGTTTCAATCAAGAAACT
TAAATATTATCCAATTTAATTTTGAAATATGAAATAATTTAAAAAATTATTAATAAAAAACATGAAAAAA
AAATGAGTAGCAAAAAAAAATTGAGCTAAAATCTGATAGAAAAAAACTCAAATAAAGATGAAATTAAAAA
AAATATTAAAAACAAAAAAAATAAAATTAAAAGAATTAAATCCAAACTTGAAAGATAAAAAGTCACAAGG
GATGAAATTAAAAATTATTTGTAATATGATAGATTATTTATAGATTAAAAAATAATTGGGATGAAATAGA
AAAAAATTGATTATTTTTAAGCATCGATTATTCGATTGAAATTCATGTGAAGTTTTGCCAGTGAGAATTT
ATTGACAGATAAAGAAATGTGCACGGAGTCTATTGGCGGACGATGAAACACGTATTTCTCCCCGAGCAGA
CAGTGCTTGTAACGTCAACTCACTCTTTTTTCCTCTCATATGGTAAGCATCTTTCAACGACGAGGGGCCT
GTCTGGCCATATAAATTGAACATTTTTTTTTAATCCAACTTTCAAGAATTGGAGCGTTTGATTACAAATC
TGTTTTCAGAGAAAGCACGCGTGTCACTCGCAGAGAGCAGATAAAACACTATCGATTATTATTAATACAC
TTATAAAAACTTGGGTGGAAACACTGTTGTACAGTGAGCCGTGCTAATTTTTTTTTTTAATTTGATATTT
TTATATTAGATTAATTTATGATTGATTTTTACAGTTTATTTTAATTGACTTGATACAAGATTATTGTAGT
GTCAGGAAATAAATGATAGCTTGGTTGATATTTGATTTGGCAAAATCTAAGTCAATTTTATTGATTTTTT
TTTATTGAATCCTTTCAAATTATTTTTTTCAGCATTTGATTTATTTGGTCACTTGACTTTGTTTTCCTTA
CAATCTTATCGTTTTACAATAGGTTTGATCCAGAGTTAAGCTTGATGATTTATTCTGATTGGTTTGACAT
GAGATTTTTGTGATATCATAAAGAAAGTGGATGCTCAATTGATTTTCAATTTGACAAAATCAAATAAAGT
TTTGGTGTTTTCTCCCCTGTTGATTTTTTTTATGGTCAATTTAATGAGTTAGTAGTAAGATTTGTAATAT
AAAAATAAAATTCAATGAAAGAGTATTGGGATAAAAAGCAAGTTAAAACCACATGTTCAATCTAAAAATT
CACAGAAAATAATTTTGGTTTAAAAATCATTTCACAGAAACTCTCTGTGTCGGCATCGTTGACGGCGGGA
TTTCTTACAGGAAGATTACCAAAATAAAAAACCGATGGTTCCATCTGACTGCTAAAACAGAAAAGAAAAG
AAAACAAATACAAAAAAATATGAGCATTTATATATTTAGTTAGATAGATCATTTATATATTTGGATAAGT
GTACTCATTAAAAAAAATTCTTGCTCATTTTTTTTTTAATTTGAATTTAAAAATGACAAGGTAAAAAGCA
TTTTATTTTTATAGCCACCAACTTAAAATAACGAACATTTTAAAGTTGCGGTGATGATAATTTAAAATGT
TTTTTTATTTAGAAATATATTAAAATAATTTTTTTTTATTTTTTAAAAATTATTTTTAATATCAACACAT
TAAATTAATTTAAAAATATATAAAAAAATTATTTTAAACAAAAAATTTTAAAATTTTTAAAAATACGAAA
CATACTGTTTCGTTAGATTACTATCCATTAATGTTAGTTTTTCAAAAAAAAAAAATTAGTTTGAAAGATA
ATTTTATTATATATTAACAAATATAATTACCAAAATAACTCTGTATAATTTAAAAAATAAATATTTACAT
CACAATGAAAAAACTATTTCATCCCAAAACAAGGGGACAATAAAGTCATGAAACTATTATAATAAATAGT
ATTCCATGTCTTGTACCGTGACAAGGTAAATTCCCAATGAGAATTAATTAACGTTTTTGATAACGGTCAT
GTGACCTTCCTGTGCAGGTATATGTGCTGTGACTTCAATGATTCACTGCCATTATGTTGAGGTCTCGGGC
TTGTATGAAAATGTATTTATGATTGTATTTTAAAATATTTTTTATTTAAAAAATATATTAAAATAATATT
TTTTATTTTTTAAAAATTATTTTTGATATTAGCGCACTAAAATGATATAAAAACATTAAAAAAATTTAAT
TTAAAATTAAAAAAATTTAATTTTTTAAAAATATTTTTGAAATATAAAAACAAAACAAAAATATTATTAT
GTTAGTGAATACTGTTTAATAAGAGGTGAAATAACAAATATAGAAAGGTTGAAGGGAAGTTTGGACACAT
TAGCCTTTTCATCATGACAATTTGTTCGAGAAGAATTTAAGAGTGAATTGAATTTTTATTTTTTTTTATT
TACTGGGTTAAATAAAAAAAGGAGACAACATGCATCAAAGCTACCCCACATTGATTATTAGGAATTACCA
CAATTTATCCATCAATCTCCACGGCAACAGGTCCAGCCCATGTGAAAGAAAATCCTTTTTTCGGCCCAAA
AGAGAATGTTAAGATATTCGTTTTCACTGAGCAGACAACACGACGTCGTTGTCTACCTGCATTCGGCAGC
ACGCATTTCCACTCAAAATCCTTAAACCGGAATGTGGACACAAACAACACTTCTACGCTCTCTCACGCAT
TCCTTAAGCGCGTGAAACAACCCCAGTGTAATAATAAGTTCGGATTTAGCAATAAGTGCATCTATGCGAT
TCAGACATAGACATGGCTATAAAGGTCGCAGGATGATATATTTGGTATTCAAAATTATTCTGAGGGTAAT
CTAGGAAGAACAAAATATCACTTTAGTAGTAATATAAATATATCAAACCCTACAATCTCTCCAATCCGTC
ATTAGCTACTCTCCTCCTTCCATCTCTCTCGCGGCCAGGGTTTAATCCATTCTCTCGTAAGGTTGGATTT
TCTGGTACATGTTATCTTCTCTTGATTTCCGTTGTAGTTTTGATCAAGATTACATAACTGAGTTGCTGAT
TAGTGACTTATTTGTCGAAGATTTGTGATATATTTCTCGGCTTTAGTCTAGAATTTTAAGCTGTATCCTG
CGTGGATTTTTTTCGTTAGTTTTTTTTTAATTCTTATTATTTTTTATTTCGTGTTATAAGTTGCTGCATG
AAATTTGTTCTTGTTTGACAGATAGATCTAAGAAATTAATCGCTGTGATATAAGATTTTTCGAAGATAAA
TACCGTTTTATACGTCCGTGTTATTTTCATTGGCATATTAAAATATGGTTTTATATAGATTTCTTTATGT
TTGTTTTTATTGCTTATGCTTTTACTTGGGCGATGATTTTGTTAATATTTGAGATAACAGACTTGCAGTT
TTAACCCTGTAGTTTTGAAATTGAAGCTGGAAAAAATCTGGGACGATTTTATGCCCTAATTATTATGGAT
AATAGTTATTAGATTTTCATGCCGTATTTTTTAACAGTCATGCGGTATTTACGAATAACTATTATCTAAA
CAGTTCTTCCAGTACCGCCTTTGTTTATTTCTTAGTTCTCTTCTGGTGTTTAATTAATAACACTTGTATA
TAATCTGAAACTATTAATTTGATTTGACTTAATACTTTATGTTGAGTATGCTTTCTTCTTGTAATTTTTT
ACTCTACCATTGATTGTTTAAACATTTCATATCTTCTTGTATAACAGTTCAGCTCTAATATATCATC
Sequence ID: 97
Sequence Length: 3642
Sequence Type: DNA
Organism: Poplar sp.
ATTTTCATTAAGTGGAGAATGATAAAAAAAAAAAGTATTGGAAAAAAAACTTAGAAATAATAAGGGTTTA
ATATAAAGAGTGTTGGGCCCATGATTAATGGAATGCAACCTGTCCTTAGCCAACGGACCAGAACAAAAGC
TGCCCGAAAATAAAATAAAATGAACAAGAAATAATAATGGTTTTTGTAAGTTTTACACATATTAAATAAA
AGATAATAAAAGGTGAATTCATCGTTTTTCTTCTCACGAAACATTGTTTCACGGAATGGTATTTTGATTT
ATTTTTATTTTTTTTCCTTTTCTTTTTTCTTTTCTTTTTTTAATTATTTTTTTTTTAATTTTGAATTTAT
TAAGGTTTAAGTTTTATAATTTGTTTTTATTTGTTTTTTACATGGTTATCATAGTTTCATGCTATAGGTT
GTGGATTTAACAAGTTAATCCGAGTTGATTTAAGTTATTTTTTGGTTATTTTTTAATTAATTTTTTTTTA
ATTTTATTCTTTAATATTAAGTTGATTAGGAATTATTCTTTTATAATTTATTTTAATTTGCTTTTTATAA
AATTATCAAGGTATTAGAACTAAGATTACGAATTTGTTAGATTAATCCGAGTTGATCCAATATAATGCTA
TCTAGACATTAAAAAAAATGATGTCAGTTTGAAATTTTTTCGAGTCGGGTTGTCTAAGTGATTTTTATAT
TTGTCAAGTTGACCAAGTCATATCAGAACAACTCCACATCATTTAATTTTTTTTTTATTGTAGAACATAT
TAGTAATATCTAGATATTATTTTTTTATATTAAAAAAATTTAATTTGACCAACAATATAATAGGCGATTG
AACGATAAATTATTTACTATTAAGTGATTTTTTTTTTTTTAGTTCTTAGTTTAACGTGGGTGTCCGGGCC
AGCTTGCGCGCACCTCGACTAATCCCACGGGCCCTGAAATTAACGACCATGTAAGCCTCCAGTGGTCATC
ATATGAGCAATCACAGGGCTCGAATCTGAGACCACAGAGGGAGCAAACCTCTTGGTCCCAAGCTTTTATC
ACTGGGCCACCACCTAGATGGTTACTATTAAGTGATTATTAATAGTTGCTGTGCTTGCTATATATGACTA
GTGGCTTCCTAACACATCCAATTTCAGTGACAATCATTCTTCTCAAGAAGGCGAAAGATAATGAATTTTC
CTGGAGGTGTGTACAGGAGAATTGCTTGTGGTTCTTGGAGAAGATTCCTTATTAGAAGGGCAATCCAACT
TACTATTGTTGCCATGTTTACAATTTCAAGAACAATGCAAAATCTTCTCTGTCAAAGGCTGCGCCATACT
TCCGGTTGGTGCCATTGTCAAAAGTCTCTAGAGAGTAATTAGATAGGTTGCATGGAGGTATCATCCTTGT
TGGCTGCCGATCAATATCGGCGAAAGAAATCAAATGGAGTATGCGGTGAAGGAAGAGTGAGCCATCGACG
GCGCTTTGGAGGCTGTAGTCGAATCAGCTGCTTCTATGCACTGGATCCCATCCCAACTTGCATAGCAGTG
TCTTTCATTGCAGATCTGATATGAAAAATGTTATGATGTTCATTCTATCAATACTCACTTTGATTCTGTA
GCGCACATATCTTTTCTACCGAGTTTACAGTGTTTGAAAGTGTGATATTAATTGTTTTTTAAAATAAAAT
TTTAAATGTGTAGAATCTAAATATATTAAAATAGAAATTCCAAGTGATTTTAGACCCAATCGTAAGGTTA
TGGACTCCAATTTCATCATCATCCTTGGATTGGCAGTGAGGTAATGATGATTTCAGACCCAATCGTAAGG
TTATGGACTCCAAAAACAGGGCTTACCATTTTTTTTATTATTAATATTAAAATTATACTTGTTAAACTCA
ATACCACCTAATAAATTAACTTGAAAAATTCTAAAACTTGATTCATTTTAGATTTTTAATTAAATTAAAT
TAAATATTGATTTAATTAAGTTCGGTTAATCTAGTCAAATCTAATCTAAACCTGGTTAAATCAATATAAA
AAAAATAAAATAAACTTGTTTTGATAAAACTAAAGAACTCAAACACTCCCAAGATGTTTAATAACTCATA
TATATATTTTCAGATATTAGTTTTGAGAGAGTTTACAGTTATGATTAAGAATATATTTGGATTAGAGGTA
TGATGGTTTTTTTAAAAGAATATAACATCTTTTAAACATAAATTATATTTTTAAAAACTAAAAAAACATA
TTTTTTACTTAGAAAAAAACACAAATTACATCTCATCCAAACACACCGTTACAAAAACCACACTGTTACA
AAAACCCATTAAGAAAGCAACAGAATCTCATTGGTTAAGCTACGGCTACGCAAATGAATGTCTCTTTCGT
TGGCTGAGATGTTGATAAAGCCAAGTTATCGTGAGACAGAATCAATTAAATATATAGTGAGGCAACCAAC
AGGAAGATGAGAATGGCTATTCAATACGTGTTTGGTAATGACTTACAAATATTTTTTATTTAAAAATATT
TTTTTATTTTTAATTTTTAATATCAAAACATTAAAACTATTAAAAACATCAATATTGTATTTTTTTTTAA
ATCAAATACACTTTTAAATATCATCTAAAAACAAAAATAAACAGTGCCTTATTCTTCTAATTAGCCACAC
ATATTTGCCACCTTCCAATATGCCTTTCTTTTCGGTTTTTTTTCTTCGATACTTCTTTTGTTGTGATATT
TTACAACGCTTAATGTTTTTTTTCTTAATTTTTAATTAATTTTTTAGATCTTTTTTGTTTTTAATACATT
ATATCAAAATTATAAAAAATATTTTTTAAAACAATCACTTTAAAAAACTGCATGAAACACGAAAATAAAC
GGTCCTTCGAAAATCAACCAAAAAATGTGGGGTCTACAAATTGAATATCAATTTTTACCCTAACCTCGTT
AAATCACTACGCCGTTAACTCATCGCTTCCATCAAGCAAAGATCCATAAATAGAGCCCGTATCTTTTCCA
TATCCTTCATTTCTTTCTGGGCAGCTTCTCTTCTCTGTAGACCCTGCCCTCTCCCTCCCTCCCTTTCTCT
GTAAGCCATCGCCATGTCTCTGTTCCTATTGATTTTGATATCGTTATTGTAGATCTTGATCTGGGCTTTG
GTCAGATTAGTCTTGTTTTGGTTGTATAGTCTGTTTTTTCTTTCTTAAAAATGGTACATAGATCTGACCC
ATGTTAGATTAGATCTGTTTTTTGTGTGTTTGTTGAGTTTTTATATGACCCTTTTTCTTGTTTTGTTTGC
TGGTGTTGATTTAAGAGATTTCATGAATCAATAAAATTCGGGTTTGGTGTGAAATATTGAAATGAAAATA
AAGGAATTGAAAAGAAGACTTTATCATGTATATAAGCTTGGATCTCTTGATTTACTGGTTTTTTTAAATT
CTCTGATAGGTTGAACATGTTGTTTGTGAAACAGTATGAAAAAGAAAAGTTTTTTCTTGTGTTTGTAAAT
GTTGGTTAAGATCTTTCTTGCTTGCATGTATTGTTTATTTTGATTGATTGACGTGATCAATAGAATGTTA
TGGAAGGTATTAAAGGCATCTTTTTTTGAAGATTGATTGTTGGGATTTTGATGCAGGCTTGATTTAGTAA
AG
Sequence ID: 98
Sequence Length: 3699
Sequence Type: DNA
Organism: Poplar sp.
TTGCAGCTTTAGATAGTTGTCAGGTATTTGAGAATCATAATTTTCTAATATAAATGGAAACTGCTCCTTT
TCTTTGGAGTCTTTCATGGCTTCATTCTGTGTGTTTCTTACAACTTAGAAATACATTGTCAAAGACCATT
GTGGTTGCGATGCAGTTTATAATACCCTTCGAGTATGCTTGTCTTGTGTAGGCTGTGTAAAACTACAGCT
ATTGGAAGTACAATTGTTTATAGGCAAATAGAAAAATAAGAAAGTGTGAACAAATGAATAAAGGTTATTT
TCTTCTGATGGTATTATACTTGATAGCTCCCAGAGTAAATCTGTATTTCATATGTGGTTAGAGCTGTGTT
AACATGAAATCCCTTAGCTGCAATTTTTTTTTCCTGAGTAATTTTGATTTCACAAGCTACAACAGTTTGG
ACTCGGATGGTAGGTTTTAACATGTGCCTTGTATTTATTTTCTCCCTTGAAGTCCCATGCACATTTTCTT
TCCATGTGACTTCTTACTCGGTTACTTGTATGGACCCTTTAAAGGATTAAAATGTAAAAATTATTAATTG
GAGCACTAGCAATTGAATAGATATCAAATAAATTTGGGTTGATTTTGGCAAGCTCTCTTGTCAGAAACCA
TAGTTCATAGCTGCATCTAAACCTCACCTTGTGAACTATCATAGAGCCACGCTATATGCATATCTCTTTT
GCAGTCACACTCACCTTGGAGTTTAATAAAACTGAAATTGCAATGTGCAAGTCCAGGATGCAACCTTGCA
ATGTGAAACCAAGCTTGCTAACCATTTCTTGGGATGAATCTAAACGATTGTGAATGTGATTTTCTCCATT
ATAATTTAAATTAATTATACCTATGACTGTGAAAAGTAGAAAAAGGTAACTTGATCTCTGAATTAGTAGA
TGGCTTAGATATATTATCAGCATTTCTAACGAGGTTTTGTTATTTTGCAGTGGTACCTGTTGCAGATGTA
ATAAGAATTCGCACTGGTAAGCCATTGTTTTCTTCCAACACTGATGCCTTTTTGTCTATACTATTCTGAT
GGATCAAGCCATGCATCTTCTTAATTATCCAAACCTTCCTGTTCTGCGATTATGATGATATATGACAACA
CTCTTTCCTTCCATGCACCATTTTCAGGTGAACGTGGAGAGAAGGCTGAAAGGATGACAGGAGGGTTGTC
TGACATGACATTTTCTGCATGACTGGTTTAAAAGCAACATGCGCAGCTCAAGATTTGCATCTAACGCTTT
CTTAGTAGCTAGTATAGTCAGGCATCCATCCTCCGCACCCCTTGTCCTAGTCCTTACTAGAAAATTTGAT
TCCTCCTTTTTTTAGCAGTCTATCTTGGGTTGAACGACTAGAAATAAAAGGAATGTCTATGAGCGTTTGT
TGCAAGGAAGTAATAACAAGAAGATGGTTGTGTGTGATCACACGAAGGATTAGGTCGCCCCACAGTTGGC
ATACGAAATTGTTGAGTACATGCTGTGGGGTTTGTACTGAGCTATGAATATTAGTGTTAAACTGTATTTT
ATTCGCCGGTGGACACTTCCCTCATTGTCTCATCTTGTACAGCATGTTCTTATTCCTTTTACCTAGGCAT
TGATGGTGCTACAGGTGGTCATTGGATCATGTGCTTTCTTAGGGATTCGATAACCTGAGTTGAAAAATGG
TTCCATTAGAGAGCGGCGTGATCTGTAGAGACTCATTATTGCCTCATTCTATATACGCAAATCCTGGATT
GTTGAACCTAAAGTGATCCTATGCAAGTTTAGATTTGATTATATGACTCCCTTCTGCTATTTTTATCCGC
GGCCCCTGTTTGGCATTGCTTCAGGGTTAAGCAAAATTTGATTTTTTGTTTTGTTTAAAATTATTTTTTT
ATGTTTTGTTGTACTATATTAAAAATGAGTTTTAAAAAATAAAAATATATATTATTTTGATACATTTCTA
GATGAAAAATACTTTGAAAATTGCACCGCTTTTCCTTAATTGTTTTAAAATTTTATTTTATTTTACTTAA
AATTAATATTTTTTGATGTTTTTAGATTATTTTGATGCGTTGATATTAAAAATAATTTTAAAAAAATAAA
AAATATATTATTTTGATGTATTTCTGAGTAAAAAGTATTTTGAAAAGCAATCGTAATCACATTCTTAACG
CCTTAAAACCGGTTAATGGTAAGGGAACGAAGCTCTTATTTATCATTGTGTTAGGGTTAGGATTTGACAA
AAATTAATTTTTTTTTTATGTTTTCATGTATTATATTAAAAATAAATTTTAAAAACTAAAAATATTATTT
TAATATATTACTGAGAAAAAATAATTTAAAATAAAAATAATTATTATAATATTTCCAAACATTCTCCTAG
TGGATTAGGAGTGGCACGCTCCTAATATAAGGTCTTGCAACAATATTTTGATTGGTTGAGGATGTGTCTA
GTGAAATTTCAAGTTTTCCTTTCATACATAAATATATTTATTGACTTTTAAAATTGTCTAAAATTTACAA
GTTAACTTGTGATTTCAATAGTGTTGCGTGGAATGCCATTCATAATATTAAAAAAAATATGAAAATTGTT
AACTTATTATTATTATTATTTTATTTTAACTTTTGAAAGAAAAAAAGTGTATCAACGGATTGATGAGATC
ACTGATTCGGCAGAAAAAATAATAGTAAAAAAAAAATTGCATTCTTCATGCCTGAAGTCTGACAGGAGAC
TGAGCTTGAATTAAATAAGAGCCTGATAACAATGACGCGAGGCAAAGAGTTTCCAAGTGCCGGGATCAGA
AAGGCCCGATCCCGAAATGGGAAACACACCTCCTATTTCCAAACAGGGTGCAATCTCCAACTAACGCAGG
CCACCGCGTCGTGTAAACGGCGTAACAAGGGTAACCATCCATTAATAGGCAACCACATGGTCACAGATAT
TGACGGCGTATTGGACAGTTAAAAAAAAGAAAAAAAAGGAAAAGGATCCCGTTACGTCCTATAAATACAC
ACCATCAATCTCTTGGAACGTGAAAATATTTTTCCCTTAAAGACAGCTCCTCCTCCTACTCTTCAATTTA
ATTCCGCTTTATTCTCTCTTCTGAGGTAGATCCCTCTCTCCCTCTCTCGGCTCCTTGATAGGCAAGTGTT
TAACGATTCCGAAATTCCGAACTTTAGATCCTGATCTTGTGTTCGATCACTCTCTTAATCGAATTGTTGA
ATGTTTGTCTGTTGGATTCTACGTCCTGATCTAAGATTTAGAGCACGATTTGGTTTCGGAATGGCTTGAT
CTTGATCATGATGTGGTGTTGCCTTATGGATCTTCATATTATGATTATGTTTTTGCATGATTTGAATCAG
GTGATGATACCTTAGATTTGGAGATCACTAGATGCAACACATCCTGTTTGGTACCTGATCTGTTAGAAAT
GCATGTCTAAGCTGTCGTTTTAATGTGTTTTGAATTGAATATCTGAGTCTTGTAGGTTCATCGTGATTTT
TTTGTCTTTCGTATGTGTTGTTGATATGGCTCATTTATCAATTAGGTTTCTGATTTGTGATTGTTTTTTG
AAATGTGAAATGTTTTAAATGAAGCTGATTGAGATCTAGTTTTTGATAATGGTGTGGAATTGCATTTGAT
TTAGGCTGGGAGATTTATATTTAGTGATTTGATTTTTTGATATCGGCAGACGTGAGAAA
Sequence ID: 99
Sequence Length: 3500
Sequence Type: DNA
Organism: Poplar sp.
AATGATGTGTTGTTGAAGTTTTGGTTGTTTATGTGCTTATCGACGGTCTTGTGCTGGCTCGTTGAATTCC
ACCAGCACATAACATATTCGTAAGCTACATTAATGAACCCACCTTGTCTTTCAGTTCTCCTGTCTTTGTG
AGGAAAGCTTTCAACGGTGCGGTGTACATCATAAATCTTATATCCACTTTCATGTTGTTAATGTGTATGA
TTATATTTCGTGCAATGCATGTATTTGTGCATAGGTGGAATTTGGTTGCATTTTTTTTATGCTGATCAAT
CAAGGATGTAGTTAAAGTTGAATTTGATTAGATGCCTTTTCATTTGCACACAACAAATACATAAGCTATA
TTGAGTAGAGCCATCTTGTCTTTCATGGTTTTTCCTCTTGTTGGAATGAGGAATGACAACCCATTATATG
GCAGATGTTTTGTTGTCTGTGGTTGTAAATGTTGTGTGGTTGAAGTTTTGGTTGTTTGTGTGCTTATCGA
CGGTCTTGTGCTGGTTAGTTGAATTCCACCAGCACATAACATATTCGTAAGCTACATTAATGAACCCACC
TTGTCTTTCAGTTCTCCTGTCTTTGTGAGGAAAGCTTTCAACAGTGCGGTGTACATCATCATAAATCTTA
TATCCACTGTCCTGCTGTTAATGCATATGATTATATTTCGTGCAATGCATGTATTTGTGCTTACATTGAA
TTTGGCTGTGTTATCATTTATGCTGATCAATTAAGGATGTAATTAAAGTTGAATTGGAATGATGTATATA
AAAAACAACCTGCTCATGTGCTGTCTGTCACCCAGGAGACATTTTCCGTCGTTCTATCTTAAATATTTTG
GGCATTTCATTTTTATCATGTCACATGTTTGATGGTTAAGGAATTGTTTTCAATTTGCTTTTCTAATTTG
CACAGATCCGACCATTGTGGAGGCATTATTTCCAAAACACTCAAGGGCTCATCTTTGTGGTTGATAGCAA
TGATCGTGACCGTGTGGTTGAAGCTAGGGATGAGCTGCACCGGATGTTGAATGAGGTAGTCATAAGCTTT
AACTCTCACTTCTTACTTTTGCTTTTGTGGTTATATGCTTTTTAATAATGGAAATTTATTGTTTGAATAG
GATGAATTGAGGGATGCTGTGCTACTTGTGTTTGCAAACAAGCAAGATCTTCCAAATGCTATGAATGCTG
CTGAAATTACAGATAAGCTTGGTCTTCATTCACTCCGTCAACGCCACTGGTAATTGATAACATCTCCATC
GCCAATTTCTCGATAGTTTGCTGCTATGTAAATATCCGTGGATGTCATCTGAGCTGTGTGTTTATTGCAG
GTATATCCAGAGCACCTGTGCCACTTCTGGTGAAGGTCTCTATGAGGGACTGGACTGGCTCTCCAACAAC
ATTGCTAGCAAGGTAGGGTCCTAACTCGTGTCTTGTAAGAAATAATGATAAAAAGTCTTCGGGTTTTGTT
GGATGTAACTTTGTCTGCTGAATCGTGGCAGGCTTAAGAGATGGGTCGATATCTGCAGCTTTTCGGGCAG
CATTCTGCTTCAATTTGGGAGACATTGTTAGGTTGTTTGGTGTTCAAGGATTCCTTATCCTGTGAGGTTG
TTAAGAGAAGAATGTATTTGTTTGGTTTTGTAAGATATTGTTACCGTAATAGAAAGTGGCAAATTATCCC
TAATAGATACTAAAATAATTCATGATTCACAGTTTTTAACATGATGTATCTTGATTCAAGTCAGGTTCTG
TAATTCATGCTATTGGGGCTTTTGTAGAGTATGGTTCTTTCATTGACCAGCCATTGCTTGATTCCGGCGG
CTGCCATTTTAAATTACAATAATGCTGTAAAACTTTGACCGAATGCATGTACATCTCCCGTGTTGACAGC
TTGCCAGTCTTCAGACACGCATGTATTTACATCTAGATTCTGTTCATGGCACCAGGAAAAGTAACATGGA
AAGAACCGACTTCTGAGAGAAATTTCAGTTTGTGCCTCTCTACATCATGAGCCTAACTTTTCCTATTTGG
CCGGCCAGCTACTTGCGTTATTAACGATTCAATGTTTTTTATTTTATTTTATCAATTCCAATAGATTTAA
TATCCAGCATAACCCAATCTAAATCAATTCATAGTTAATTTGTATTTCTTCTAAATTTCTAATCTAAAAT
TCATGATTTCTACTTGAAATACTATTCAAATTGCAAGAAATTGCTGAAGTAGATCTAGAATCCCCTATAA
GATATTATTCAAGTCTTCGAATCTCAATTTCTTCCTTGTTATCGTGACTATGCAACCCCTCTGCTTTGAT
TATTCTGCTTCTCTGATTTTTAATTAGGTGTTTAGAATTTTCCTATCTTCCCTTTTGTTTGCGGTTGCCG
GAAGAGAAGAGAATCCCCTCTTTTTTAGCTAATTTACTCTTTTATCTCTTCTTAAGCATAATATGATTTT
TCCTTCCTTTTTTTTATTTTTTTTTTATTATTTTCTTGATGCCGCACTGTTTTTATACCTCTTTCAATTT
TATTTTTAATATAAAAAAATAATTTTTCTAAATCTTAATTTAATTTTTTTTATTAGTCAGAAGTTTCAAT
TAACACTACTTACTCGTGATCAAAATCTTAAGGTTCTATAATCATGGTTAGAGGTATCAATGAACATTTT
CCTTCCTTTCTTTCTTTTCTTTTTTTCTTACCAATTGAACTCAATTTAATATTAAAAATCGACAGAAGTT
AGCTACTAAGTAATCAAAGATAACACAAAAAATAATTAATTAAATTTAAAATACATTAATAAAAATAAAT
AATGCACACAAACACAATGGTAAAATTTAATATTTCTGCCAAATTAAATAATAAATGAAACTAGTAATTA
GAAATATAATACATCCACCGTCCCAAAATAAAAGTACGGTCCAGAAACTGAATCTCACTTTGACACTAAC
GTCATTAACGGCGTTAACTCACGTCCATGGCTAGATTCTATACGCACATAGATAAATATCCCCTTCTCTT
TCTCATTTCTTTCATCTCCCATCTCTGCACAGCTTCTTAGTAGATCCTGCCCTCTCTCTTCGTCTTCGTT
CTGTAAGTCGTCTCTCTCTATATATATATTGTTGATGTTATTACTGTAGATCTTGATTGGTCTTATTTTG
GTTCTAAAGTTTGTTTTTTTGTTTTTGTTTTAAAGGGTTCATAGATCTGACCCGTGATAGATTAGATCTG
TTTTGGGATGTTTGTTGAATCTTTTGACGACCCTTATGCTTGTTTTGTTAGTTGGTATCAGCATAAGGGA
GATCATGAATCTATAGAACTTGGGTTTAGTATGAAATAATGGAAGGGAAGTGAAGGAATTGAAAGGAATG
GGGTTTTTAATTTGTTAAAGTTTGGATCTTTTGGATCTGTTGATTTTTCTATGATTTGTGAGATGATTTT
TCTTTTATTTAAGTCTGAATTTGGAGTTTTTGATTTACTGATTTGTTTTTGATGCAGGTGAGAAGGAAAG
Sequence ID: 100
Sequence Length: 3114
Sequence Type: DNA
Organism: Poplar sp.
TATATATATTAAAAAAATAAAAAAAATAACTCGAGTCAATCTGAATTCATCAATTACCAGGCATTTTCTT
GTCCTACCTTATTGTAGCCATTAAATATGCTTGTATCATGAATATAAACTAATTCCATTTAATAAGTTGG
TCAGTTTTTTTTAATCATATATTTAAGAACAATGGTGGTGCTATCAGAAGGAAGCTGAGAACACAGGAAA
CTCAAAATAGAGCCAATGAACCCATGGTTTTGGCCATGTGCAGATCAACTTGGGCACGTCTGTATATGCA
AGTTATTTATTTGCGAGACGTGGACGTTGTCTGATTCAAAGTTGCAACTAGAAATTTAGTATTCGAAGCT
TTACATTATTTTAATTATTATAAAAGATACGTGGTATACAATGCCTTTTTACAAGTAAGGAAGGTGTGAC
GACGACGTCTTGGACTTGGAGTTGCTCCCATATAAAATTATATTAATAAGCGAGATTTCAAGATATTATT
TATATAATTTTTTAATAATTTTAGATAAATTTTTAATAATTCATAAGAAATCTTTTTTTTAATAACCTAA
ATAGTAAAATACAAGAAAAATTTATTAAAATTCTAATTAAATTAAAAGTTTTAATCTAAATAAAAAAAAT
CTTAAAACAATAATGAAAGAAAAGATAATTAAAAAACAATAGCTTCTTAGCCTAAAGCTAGCGGAGGGGG
CGTGCAAATGCAATTGCAAAATTGATAAATTTAAAAAATAATTGTACTGATAAATCGGTTTTTGGTAGTT
GGAATTCGTAAAATTAACTTTGAACGATATACATAAATTCAAACTCATCGTGTTTCAAAATTCAAGTCAA
CTTGTAGCAAGGGAAACATTGTAATGTAATTTTTCATATTAATTTTTATTTAGTGAATTGAAGAAAGGAT
TGAGAAAAGAGTCACGTATTTAATTGAGTGCCTCCTTTTCCTATTTAATACTTCAAGCTTTGGTTTTTGA
ACACCACTAGATAATTTGTATGCTATATGTTTCGGATTCGATTAATATTAAAAGATATCTAAGTGTTGAT
AACATGTTTTCTAATATAAAAAAATTTAAATTATATAAAGGTTGATTCGGTTAACTTGATGGACTCAAAT
TCAAACCTCGAATTATGAGATTGAGATAACTCTAATATAAAGTTCAATTCTCAATCAAATCGAAACAAAA
TATGAAACTTAATTTCTAACCAGCCTTATATTAAAGTCTAGAACTAAAAAAGAAAATTCAATTAAAAAAG
ACAAAAAAAAAAAACAACTCGAGTCAACCCGGGTTCACGTGCCAAACTCGCAACCCAGGTCATGTGATTG
GGATAACCCTGTATAAAGCAAATTGAAATAAATCATGAAACTCGATTCTCAATCAACCCAATGTTGAATA
ATAAAATTTAAAAAAATCGATTAAAAAAACTTAAATCAACCCACCAAACCCGTGACTCAATCAATATGAA
GTTTAATTCTAAATCAATCAAATATTAAATAATGAAATTAAAAAAATAAATCGATAAAAAAAACCTCAAG
TTAACTTGTCAAACCCGTGACTCAGGTTATGAGATCAAAATAAATAAATTTAATGTTAAAGGATAAAATT
AAAAAAAAAAAAACATTGTTCAAGTAAGCGGTTCTATGTAAGGAGAGTACCAACGAATTTCTCTTCAATT
AATTTTTGTCAATATTTTAGAAGTGTAACATTTTAATTTTGGGGTTTGGGGCATTTTTCCAAAACTATTT
TGAAAGCTTTTAGAAAACCAGCATCAATTTTAAAAAAAGTTTTAAAAATTTAGGAGTCATTGTTACTCTA
TCCTAACCCATTTGAAAATATTTTTTATTGTAAAAAGAAAAAATTGTATACTATCTTAGATTAACTACAC
AATTATTCATTTCCCTGAAAAGCAAAGAGGGATTTGGATCCTCTCATAACCCTAACAAAATATAACTAAC
CATGTTTTAAATTTCATCATTAATTTTAAATTTAGTATTTTAAATCAGACGAGGGGAAAAAAATAAAATA
CAAGTTATTATTAGATAGGTTACACGCGTTATGAAGTGATAAGATCAAATTTTTTCTAATATAAAAAAAA
TGTTTAGGCGAAACTAGCTTTTTTAAAAAAATAAGAAAAATATAAAACATGAGTCATTGACTTGACTAAG
TTCAATAAACTCAATTAATTTAATAATATGTTTAAAAAGAACATCGATAGTGAATAAAACAAAAGAAAAT
AGCAATAAATAACTATAGAGAAAGGAATAGCCAATATTGTACTCGATGAGAAAACAAAACAAGCTCATTA
GAGACTCGTTACTAGAAAAATCTCATCGAGATGGTTATAATGTTATTGCGAAAGGGCGACGATATTGAAA
AAGATTGCACACACCGTAAGAGTAACAACCTAAAAAGCGAGCAAACAAATATCATGAAGTGAACATCTCA
TGTTTATATTTCATTAATTAATTTAATTTAATTAAAAAAAATTCTTTTAAAAAAGCTAAATTTAAAAAAC
AAAAAAAAACACATAAGATAATCCCTTAAATCAAACGGTCCTCCTGCAATTACAACATACCGTAACTGTG
AAAACCTAGTTCCTTCCACGTCATACTAAGACCCTGTTTATTTTTGCGTTTTAAAAATATTTTTAAAAAA
AATTAAAATTTTTTATTTACTTCAAATTAATATATTTTTAGTATATTTAAATCATTTTTATGTGCTATAT
TAAAAATAATTTTTTAAAAATATATATTATTTTAATATATTTTAAAATTAAAAATATTTTAAATATTCAA
ATTTCAAAACATGTACTAAATATCGTCTCCTCCCATGCAAATTTTTCACAAGCAGACAAAACCACCAATA
AAGAACCCACAATCCTTGTCCATGTCATATCTGACGCCCGAGAATCATCGCCCCTGTGGCCCCCACGTTA
CATGTCCCCATTGTACTATACAACATCATAATAAAAACTCCCCAAAATAAATGAAATTACGAACATACCC
TCGTCAATTTCCCTATAAATCCCGACGCCTCTCCCGCTGATGATTCCATGTCCAAAGCTGTGTGTATTTC
TTGCAAAAGAAAATAATATTTTTATTTTTAAAAA
Sequence ID: 101
Sequence Length: 3058
Sequence Type: DNA
Organism: Poplar sp.
AAGGAGTATAATTTTAAACGGGAAATTATTTTCCTTCTTTCCTTTTCCTTTCCCGTACTTTTTTTTTTAA
AATATATTAAGATACACATTTACCTATCTTCAATGTTTATTAACATTCTACATTATTTTCTCTCTTTCCA
CAAGCACACATTTCTGTCGCATAATAAACCACAACCATTTTGAACTTGTATTATCAACAGTCTTTCCAAA
CATTTCTTGACTGTCTTTCGAAGTTCCTTACCCTTATGACTAAGATCCGACCAAAAGATTAAATTTCTTT
GGGATACTGTACACGTAAGTTATTATAAAATGGCTGCGCTCTTCCATATACATTTTTTAAAAAAAAATCT
CATAAACCCTTGTACATATATATTTTCTTCCAAATAGATATCTAGAAGATATATAATTAGGGTTTGCATG
AGGTAAAATTGAAAATAAAAGTTACTATCTAGTTTTATAATAACTAGTGACTTTAATTGGCATTTAGAGT
TCTAGGTAAGAGACTGATTATTTAACAAGAAAGATAATAATCATCATGTACATTTTAATTACATAAGATA
AGGTACATGTTATGCTCTAGCTAAAGTTTTTCTAAACTAGTATATGTGTTGTTATTAAAAAAAAAATTAC
GAAAACTTTAATAATGATTTGGAACATAAGCCTGACAATAAGACTTAGTCCTCATTTTTAGAATACATAT
TGAACTTTAAGTTGATTATGTCCAAATGAACTTAAAGCCTTCATTCACACTCAATAGGTCTTGGTAGGGT
TATATTCACCCAACTTAGGTTTACTTGCAATGATGGGGTAATATCCTAACACATCTAAAATTATAAACAA
ACATTGGTATACATTGATACTACACATCGAGTAAGGGGTTGGTTATGCTTTGGAGAAGATAGAAATCATC
TTTCTCGTATCTTTTCAAATGAAAGGTTATCTTAATTATGTGTTTTGTTTTAAATTTATTTTTACACAAG
CAATTATTGTCCAAATTATTTTCTAAGATTATTCGTAACTGACTTATTATTGATTGATGTTCGCTAAGTC
TGCATCAAGCCCTTAATCAAAACTCTCGCTAATCAATAATTTATTTTTTATCTTTTTTTTTATTATTAAT
TAAAAGCACTACGCTAGGCCTAAATTAATTTTTAAAATTAGAAGACTTGTTGACCTAACTATTAAAAATA
AAGTTTAATTAAACACATGATATTAACTTGATGAAAACTCTTCGACTCATTTTGAAGTTAAAAATATTGT
GCTAGGCCTAAGTCGGTCCTTAAGACTAGGAGATACGCCTATCTAGATGCTAAAACTCAAACTTTGATCA
AAACATAATGTTAAACTTGATGAAAACTCGGTGAAACATTTTTATTCTTAGTTTGAAAAATAAATATTTA
CACTCACATGAATACATAAAAAGGAATCAATGAAACTTTACAAAATGAAAGTAACACTATTGTGAAGCCA
ATAAAAATAAAAAATAATATAAAAACTGCATGAGTGAAAACTATATGATTTTTAAAAATAATTTTTACAC
ACACATGCATACTTAAAAAAGTGACAACACTGTTTTATAAAATGAATATAATGCTACTTTTAAGCCTTAA
AAATAAAATAAAATAATATGTTTGAATAAAAAGTTGTTGATTTTTGAAAATAAGTATTTACACACACATG
TGTACTTAAAAAAAGTGATAGTAGCATTTTCTAAAATAAATCTAACGCTATTGTGAAGCCATTAAAGAAA
ATAAAATAAAAAATACACGAGTGAAAAGTTATCTGCAAATTAAGACAATCGATGCATTAATTTAGAAAAT
AAAATAATGCTAGAAATGAGAATAGGATACAACATTAATTTTAACGACACAATTAAAAATTAATAAACTT
ATATATATATATATATATATATATATATATATATATTAAAAAATTAAATGTAAATTACAGATTGAGATGA
GAAATTAACTATTTTAACCTCCTGACACACTGGCCCCATCAGGGCAAGTTGTCTACGTGTCAACATCATG
CAGTTGTTGAAGTTGTGATTTAATTCGGTGATTGGATGCTCATGCATGTGAGATTTTAGTTATATATCCG
TCAAAACAATTTTGATTACTTGCCCTAGTTTGTGTACCATTTTTATTAGACCAAGAAGGGTAAAAATAAC
AGCATAATTTTAAAATTAACAATCATATAATTTTTTAATATTTATTATATTAATAACTACATGAATCGAC
ATAAACCCTTTATTTAGTTTAAGGAGGATAGATATAAGCTTTCATATGCCTTTTTTATGTTTTATAAGAT
TCCTGGCCGTCTCCCTCTTGAACGGCTTTTCAGCGTGAGGTTTTTTTTAGATTGTGTTGTTATTAATAAT
GCTTTCTCATAATTATTTTCTTATACGTGCTATTTAAGTGAAAACAAAACTACTTCTTAACGGTGAAATC
CGAAAAGAATCTTGATAAAGGGTTCAAAATTATTCCGACAAGGTGGGAAAGTTGTGGTCCCTCCATAAAA
TAACACGATGTAAAAATAATTTTTTAAATTTTTTTTTAATTTAAAAATTGTATTACATTAATTTATTTTA
AAATAATAATTTTTTATATAAAACCATCTAAATAACAAATAAAAAAATTAAATTTCAATAGAAAGCACGT
TAAACCTCAGTTCCATGTAATATATTATCTACTTTTTTTTATCAGAGATGTGTTTGGTATTATAATTTAA
AAATATTTTAATTTTTTTTTATTTTTTATTTTAAATTAATATATTTTTTTAATATTTTCAATGATTTTTA
TATATTGATATTAAAAATAATTTTTAAAACATAAAAAATATTATTTTAATATATTTTAAAATAAAAAATA
TTTTAAAAAACAATTAATATTATATTCCCGTTCCTTACCAACCAAGTTGGTAGCTGGCCACAAAACGAAG
GGCTATTTGGTAACTAACAATTCTTATGCCTTTTATAAATGATGGATACTTCATACTTGCAAACAGAGAA
TTGGCACCAGAGAGAGAGAGCCTTGTTATTTTTTGACAGAGATCGAAG
Sequence ID: 102
Sequence Length: 3252
Sequence Type: DNA
Organism: Poplar sp.
CCTCATAAGCATCATGGTGCTCACGGCACATGATCTGGGAGTGAATATCTGCTGTTATTGAACCATTGTC
AGCTTATGAGCAATGTTCTTTGGAGTGCTTTGGTCTTGCTTTATGAGAAATTGTATTGTTTTTAGCTAAT
TAGCCAAAATCGATTTTTCTTTTACTAGTAATGAAAGGGTGTTTATATAACAGGTGTAGCCTAACGATTG
GATGTATATGTAGTTGCTTTCTTTGGTGAAGCAGGTTTGAGTCTTGGTGACATTTATTTTGGTTATTACT
ATTTTACTTCTTTTGCAGCGCTACAGGTTGGGCCAAGTATTTTTTGAACTTGTGTAATTTTTTTTATAAA
AATAAATGAGGCGAATTGTCGCAGACGACAGCTTAAAATTCGGCTATTATTTGGTGGTTGGAAAATTGTG
AAATATAAATTTTCGGTTGATAATTAATTTCCAACTATTTTTCATCTTAAAATATCTAATAAATACCATA
AAAATCTTTTTAATTCACATATTAACTTTAAAATCATTAAAAAAACCAAAATCAATTGAACATAAAAAAA
TAAAAACAAAACTTAATACTCACCACAATCAAACTCTTCTTCATTGAATAGAATTAGTAGACACTAAATT
TAATCATTTTTGTTATTGGTATTATGATAATTAACCCTTTTTTTTTCATAATTTTCTCTTATCTCTCTCC
AAGTCAATAACTGAAAAATAAATAAAATAAATTTTTGAACTAAAATAAAAATTATTGTAAATTTAAGGTG
AGTGATGTATTTTCTCCATATTTTAATTTAAAACTTAATTTTATATAATTAAATAGTAAAAAAATGCAAT
TAAAAAAAATATTAAAAAAAATGGCAGGAGATTCACTGTTAATATAGATAGTGCCTGCCCTTCAACTTTT
ATTAGTTTTTATTAATGTAATAGATTTGAAATCCGACCAAAAAAGGGTTGGATTCTAAACGCTTTATAAA
TCAACTCGAGTTAATTATTTTTTATTAATATAATACTATTTTATTCTTTTAAAAAAAATACTAAAATATG
GATTAGATCAACAGAATCATAGTTAACTTTTCATGTTAATCGAATTTAGACTAGCCAATTTCATCTTTTG
TTTAAATTGAAATCCAGGCATGGCTTGATTATAGGTGGTGGGTTTTCCGAGTAAAACCCATTGGGCAAGT
TTGAGGTGAACTTATTGGAGAAATAACCAATTTAGGTCCACCAGATCTTTAAATGCAAAATCCCAAACTC
TAAACAAAGTTATGTCACTGTGTTTTCTTTTGGTAGAATCTCTTAAAGAATTTTAACAGTGTGTTTTTAT
ATTGTTTTTTAAAGTATTTTTTGTTTGTTAAAATTAATTTTTGATATCATCTTATTAAAACAATAAAAAA
ATATTAAAAAATTAATTTAAAATAAAAAAAAAAAAATTTTTAAAAAAATTCAACTACACCACAAAAACAA
ACACACTTGACACACCTAACAACTGTTTTTCGAAAAGGTAATGGAGACTTCAAAGATGGATTCCCATGCC
ACGTTAAAATCAGGAGGAAAGACTTCCTTTTCTCTTTCCTGTGCTTCTCTAGAAACTACTTAGATGTTTT
CTTTTTTAACACATGTTTATCCCAAAAAATGAGAATTTATATTTTTAGATCAATGAATCGTGGACGTAGC
TCCAAAGATTTTCTTTTTCATGAAAGTAGGCACCAATTGCTTGCAAATTGTTTTCCAAAAACTATACTCA
AATTATTCTTCCCACCAAGACTTGTCCAATCATTTTTGGAATTTTTTCTCAAAAATATCATAAACAAAAT
CTACACTTTATATTAAAAAAAAACTCTTGGAATTGCAAGATTTGCACCAAGAAATTCAGTAGATATTTTT
GAGTGACAATTAGCAAACTTTGAAATGCTAAAATTAAATGAGAGCGTTTGGAAATGTGGTTGTATCCGCA
TTTTTAAAATTTTAATTTTTTTTATTAAAAGTTAATTTTTTTTTTGTATATTTTGGATTGTTTTGATGCG
TTGATCTCAAAAATAATTTTTAAAAAATAAAAAAAATATTATTTTAATATATTTCAGCATGAAAAATATT
TTAAAAAACAATCACAACCACAATCCTAAACAGGTATAATATTGCAAAATGAAAAGGGCATATGATTCAT
CAAGCGAATTAAATCCCAATTCAAAAGTGGCTTCAAGCGTATTGTCTTCCTTTATAAGTGGAGCCATGTC
TTCATCAAATTGGCTTGAAAAGTCAATATCCAACCGGTCCGTTCCTACAAAAAATTGAGAAATATATAAT
TTAATACTAATGGGTGCACTTCTTAAAATTAATGAAAATATACCTTTTTTATTGTACCTAATTAACATTT
ACAATTCTTACTCAGAGAATTGATTTTTTTTCTTAAAGGGTTTTTTTTAAAAAAAATTAATTGATAATAA
TTTTATAATTTAATATAATTTGGTTCAATAAAAAAAAGAAGGAAGAGGGAGAGATTATTTTCCCTCAAGG
CTATTTTTACAAGCCACGAGTTTTTAAAAACGGAAGTACATAATCCTCCTATATATATATAATACAAAAA
AAGAGAGAAATTAAAAAACCAAGTGGAGTAAAGTAATTAAATAGATTTTGATGGCAATTGGTTTGTTTTT
TCGAGTAAAATTCAATAAAGGTGGTATTATTCACTAACTATGTCAGAAAAAATAGAATAGAATCCTTAAA
AAAATTTTCGTTTAATTTTTATTTTTAGAAATATTAGAGACTATTTTTATATGAAAACAAATTAGAAATA
GGATTTTAGGGTTAAAAAACTCTAAATTATGCATGATACTACAAGTTGTCTACCGCACATAAAATGACTT
GTCAAAAAAAAGAAAATTAAAAAGAAAAGAAAATGGAGTAAGGTAGGCGTAGATTAAAAAAAGAAAAAAA
AAGAGAGAGACGCAACATGATATATGAATAAAAACCAAAGTTGTTCTCGAGAACAGTCTCGCAACCCTCA
GTGTTTGCCATCTTGGAAAAGTTGCAAGAAGAATTGTAAACGACTACCTCTCTAGGAGCCAATAATATCT
CAGCTAGCCGTCCCCTTTGACCAAACAAGGTATGACCATGCACCTCTATATAAATGGTGGATACAGAATA
ACAAAACCAAGATAAAAGCAGGAAACAGAGGCTTGTATTACTTTGATTGGCAGCCATAACAGCAAACAGA
TAAAGGGATTTTGTATTTTTTTGATAGAGAAG
Sequence ID: 103
Sequence Length: 3141
Sequence Type: DNA
Organism: Poplar sp.
AATTGAAACGTCATGCAAGGAATAAAATATATTTCAGTGCATAAAATTTAAGATTTAGCAATTAATCATA
GAAAAAAATGAATTAAAAAAAGCCTGCTTTTTTCTAACCTAGGTGGGCCCATGAATTATCTATCTTCCAG
GTCTTTAATGATGTGGTCACAGAAGTCAAAAGAGAAAGATAAGAACAAAATTAAAGCAAATTGATGATGC
CTTTGTCATTTCCACATGCGTGGGTCATTACCCTTTTACTACTGAAAATTTAAAAGTTCAAGGGCCCATT
AAGGCCCACAACTAAAGCCCTTTAGATTGCCCCCACGCAGGATCGAACTACGGACCTTCAGTTTACAAGA
CTGACGCTCTACCACTGAGCTATAGGGGCTAACTATTAACCTTATCAATATTTAAGCATTAAACTCTTTT
AACACCTTGTAGTCTCATCTCCTGTGTGAGTAGACTATCATTTTTTTAAAAAAAGTGACAGTATATATAT
ATATATATCTTTTTTTTTATTAACTAGATTTTTCTGGATTAATATCGTATTTGATTCAACTAAAAACTCG
AAAAATTATGACTACGAACATTATTATAATTTTTATATTCCAATAAAAAACGAGAGACAACAAAGTTATT
TCATACTCCTAGCATGAGATTACTTACATATGTATGTGTAGTAACAGCTAAAGGATCAAGGGATTTCCAT
ATTTTCATGTTGTAATTGTGTTCTTGACAATGTATTAAGTGATGGGAGGATCAATATTTGTGTTGCAGGA
AGAGTGCCACAGCAACAGAAGAAGAAGCCAGGAGGAGAAGAGGGCATCACCAACATTAGCTTCTTCGACA
GGATCATGAGCTATGAGAATATGGTCCATGGAAAGGGCCATGAGAGAGTAGTTTCATCACCTTTATGGAC
AAGCAAGGCCATGATAGAGAGTGTAAATGAGGTGTGCAAGGATATTGCTTGGGGTGAGAGAAGGGAAATT
GGAAGAATAGGGATGGCTTTGCAAGATTATATATGCAGGGACTTGATTGAAGAGATTGTTAAAGAATTGG
GATTTGACTGCATTTATCAACTAGGTCCTCTACCGTTTGAATCGTGTAAGAAAAGACTACGTTTCTAGTG
CCCTTAGGCCCCCCTTACCATAGTTTTTGCACTGACCACAGCCTCCGTTCAAAATAATTTTTTTCTTCGT
TTGGTGCTATTTCTAAGTTTTTTTCTTTTTTTTTTCTTTAGAAGTGTGTTGTTTCTTGTTTGATGTAATC
TAGAATTATTTTTTTCTTTTTATAATATCGTGTAATTTGTGATAGCATAAAAAAAAAAATGAATGAAAGT
GTTAAATTAAAAGAATTATATTACATTTGTATCCTGGTATTTATCACTATTATTAAACTTGATATTAATC
TAAAATTTAAATAGGGTTAAGTTTCCAAAAAAAACTATTAAAAATTGATTTAGTCATATTAGATACGTAT
TAGGATAAATTACTATTTAATTAAGCTAGTTAAAACCTGGTTTAAGCTTTTAGATTTTATTCAAAATAAT
ATTATTTTTTATTTAATTAAAATATTTTTATTTTTATTTATTCAGGTCAATTCATCTGACTGATAATTCA
AATTACAAGTCAATTTATATTTAATAATTTTAATATTTATATGGACTAAAATTTTCTATTACCAGACATT
AGGTTTGAACGAATACGTGCTATTTTGTATTCCATGAGATCTGAAAGCTTATAATCCAATGAATTTAACC
AAATAATTAATAAATAATAAAAGTTGAGGTGTCCAAGTTACATGGTGAGTGTGATGTTTTAAATAACTAG
GAAAATACTATCACTACCAGTCTACCAGCAAACGACATGCTAACATTGATTCGACAAAGCCGCCTAGGAA
CAGTGCGATAGTGAGGGCGATGGAGAGATTACGTGGCGGTCAACGCGAAACACGTGATGTCATCCTCCGC
GTCCACACCGCGTAAACGCGTACGTTACCTTTTTGCCATTTTTAATGATTCGCTATACTCACCAGCCGTT
TCACCGAAAATTACTGTCGGCCAGTAATTATTCAGCAAAAATTCTTTTAAGGATTAATGAAGCTTACGTG
GCGTCTCGTGAATGGGGGATACTAATAATGGCTAGTAATTAATGTCCTGTTATTTACAGATCTGTCGAGC
TAGCTCTTCTGCTCATGATTGGCTTTCTTGGACTTGATTCCTGCTGCTGTACGATTTTACCCCTATTGAT
TATTGTTTATGACAAGAGGGTATTTTCTTCAAGTAAGTGTTTAATTTTTGCAAATGACTGAAATACCTTA
AAATAATAGTGTACTATATAATGTATTTTTCAACAAAAAAAAAACACTCTACCATAGCGGTTTAATGGCA
TCTATTAAAAAAAATTGATTTTTTAATGTTTTTGTATAGAAATTACGTGTACAGAGACAACAATTGTGTC
TGTTTAAAAATGCGATTGTGGTTGTTTTTAAAATGTTTTTCATGCTGAAATGCATTAAATTGATATTTTT
TTTATTTTTTAAAAATTATTTTTGAAATCAGCGCATCAAAACGATCCAAACACATATAAAAAAATTTCAT
TTTTAGCAAAAAAAAATTAATTTTTTTTTGACTTTTCATCCTTTGTGATAATTAAATTGAACTTGATTTT
TTAAAAAATGTTAAGCTAACTTGACTTCCTTCAATTTATAAAATTAATAACTAGATATTGATGCATGATC
CATTATTCATAAATATTAAACATCACTAACCTTGTGTTCTTGTTCTTTACCATGAAAAAAAAAATAAAAA
AAAGCTAAAGAGAAAAAAATTCTTTTGAAGTAAAAAGCTTACAATGGAAGGGCATAAGGGTCATTTATGG
ACTAATCAATATCAATTTTTTTAAAAAATACGAAATAAAAAAAGGAAGAAAAGAAATGAGAGGGGCAAAA
TAGAAAGTGAAAAAGTGAGTTGGAATTGATAAATGGGTCACGTCCGACTAGAAGAAAGCTTGGGGTGGCT
AATTTAGCTCGTATAAGAACACCACATCTTTTGTCTGAATCTAAACCAAAACTCTCCCTCTCTCCTACAC
GCACGTTTTCTCCATCTCTCAGGTTTTTATCTCCTTCTAGGTTTCATTTTTCAAAAGAGAA
Sequence ID: 104
Sequence Length: 3186
Sequence Type: DNA
Organism: Poplar sp.
GCCGCAACACGTCGACCACCATATAACAACTGTTCAATCTAATATGAAGCAAATAGAAATTATTAATTCA
TAAACAAGATGCAGCGGTTTTGTCCAAACCTATAAAAAGTAACAAATACGTATAATATTTTGGATCCAAT
GTGCATATGGATAGTACTCCATCTCCTCGCGTGGAGGAACCTGCGCTGGGTTTGGTATAATGATTTGACG
TATTATACTCATAGGTGGGTGACTTATTTATTTTTTGTGGTAAGTTTGTCTCCCTTGAGTCACCATTGGT
GACCTTTCTTTGTGTCGTTGAATTTGTCTCATCGAGATATTTTTAATGGTATCAGTAGTGTCGTGATCAG
AGCTTCAGTGTGTCTTCGAGAACTTTTTTTTTCTCTCCTCTCTCCCACCCATTTAATAATTTTTTATGGA
TGCCGTTATTCTAAATAGCGACAAGTTATAAATGTCACGTTTCTCAATCGCGACATATACAAATTGTGTT
ATTTCACCAATTTTTTTCAACTATTCTATTCCTTTAATTTTTTTAATTTACTATCCTAATTTATTTATTT
TTAAAAATCCTCAAATGGCGATCTAGTTATAAAAAAAAAAAAATTCACATCGCCACCATTTTAACTCATG
CCTTACCCTAGCTCAAATATAAAACAACATAATGTCAAAGGATTAGTTCGCAAAAAAAAATATCCAGAGA
GTGAATTGAAAAGATGGGTTAACCTTCCAAACTCGCAACCTATCCTAGACCAGAGTTTAATCAAGAAACT
CAATTTCCAACCAATTTAATATTGAAGGATGAAATAACAAAAAAAAAAGTTGTCTAAACAAAAAGAATTG
CATTAAAAAGAATGATGATCAAATCACACACAAAAAAAAAAAATTGAAGGGGGGTGAGATTGTAAAAAAA
AACTTCAATTATAAAAATTAATTAAAACAAAACAAATAGCAATCAAAAGAATGTGGATCAAATCTGACAT
ATGAAAAATTAAAGGATAATGAAATTGAAAAACAAATAAATGTTACTAAAAAATCATTAGCCTAAAAAAA
GAAAAAAAAACAAGCGAGCCCAAAAAAATCCTTTAAAATTAGGTTAATCTTCCAAACTCATAACCCAATA
AATCCTAGATCCAGGTTCAATCAAGAACCTATCTCAATTCCTAATCAATTTAATATTGGAGGATGAAATC
GCAAAAATAATTTAAATTAAAAAAAAAGTTGTCAAAACAAAAGGAATTACAATTAAAAGAATAAGGATCA
AATCACATAGGAAAAAAAAATAAAAATGGATGAAATTATATATAAAAAAAACTTCAATTCTAAAAATTAT
CTAAAACAAGACAAATAACAATTAAAAGAATGAGAATCAAATATGTCAGATAAAAAATTAAAGGAGAATG
TAATTGAAAAAAATCCAATTCTAGAAAACATTTCAAATAAAAGTAGAAATAAAAAAAAAACATGGAACAA
ATATGAAGGAATAACAAATTGAAAGATTGCTTTGAAAATTTAGAGTAACATGTGTGGAAATCAAGGTGGA
GAGAAAAAAGAAGGAAAACAAGAAGAGCAGTCCATCAATGCCAAATTATAGATCAAAACGCCACACACAT
CGTCATTAAAAATAGTCCTCTCAAGATGATTTCAACCACACCAAAACAGCTCGTTGTTAATCGCTAGAAG
CTGCTACACGCGTCATCTAAATGTGATAGAACGCCCCACACACTGACACGTATATTGCAGCCAGGTTTCC
TTTCTTAGTAATATCTATATTTAATAATATACTAAATTGTACTTGAATCAACATATTGCACATGTCGGCT
AGGATTTTTTTTCTTTTTAAAGGTATGTTTTTGTAATTGTGCTATGCTAACAGTAAGGGAAAAGCAAAAA
TGACCTTAAACTATAGCTTATTAATTTTTTAAGAGTAATTGTACTTTGCTAAAAAAAAAAAAGAAGTAAA
AAGATGGAGTTGTCCCTAGTGAATTTTATAACGACTAACAAACCATTTAAAATACCGAATTACCCCTAAA
AACAAATTCCTTGAGTTTTTTTAAAATAGCAAAAAAATTATTACACCGTAATGGTAAATAATAAACTAAA
TCTATAACTACATTATTTTTGCTTTCCGTTTTAGGCTTTTTTGTTATACAATTTGAATCAAATTATTATT
TTGATGGCAGCCAAAATCCCATTGTGGTACAAGCAGAGTTGAAAGTGAAAAAGAAATAAAATAAAGATTT
TGAGCCAAGGCAAATTTGAAAAATCCAAAATGAGTGTTTTATTTCATAATTAGATTGAACTTGCTTCTAA
ACTTTGTCATTACTTCTCTGCCATGCTTAAAATCATCATAACTTTGAAAAATATGATTTATGATATATTA
AATTGAGAAAACCAGAAAAAAAATAATTAAAAAAATCAAATTATGATAATAAATCGATTAAAATTTTAAA
AAAACTTACCGGTTAAGTTTTGGTTTTATAAGTCTGAAACCGAAAAAACCGAACTGAACCGAAACTGGTC
GGTTTGAACCGGTTTCGGTTTTTTTTAAAAAAATTCAGTTTGATTATTTTTTTTGATAAAAACCAAACTG
AACTGAAAATGATCACCCTATCATAAATAGGTATCATTTCGTTCATTCATTTTGAAGAACCAGAGGTATT
TGATAAAATATTAAATAGATCATCTTTTATTAAAAAATAAACTAACGCATTTTAAGGAAAAAATAAATTA
AAAGAATTATTTTAATAAAAAAAATCAACGAATTGTGAGTTGAGTTTTGAACGGTTCATTATCAGCTTAA
TTGAATCAATTCAAGCCTCGGGTCGACTCAGACACATCAGAAAACGGGGAGCTGACAGGGAAGAATAGTG
AGGGGTAAAACGGTAAAGGTCAAAGACGCGAGTCGAGAGAAAGGGGTTACACGTGGGGAAGAGAGAGAAT
CAGATGCGGAACACCCCAATGAGAGCACTTTAACTTTAAATTAAAAAAAGGGGCCCACGTAGGCTTATTC
CTCTGCCTATTTATTTTTCAAAGCCAGAGTCAGATCAAAGCAAAGACGTTCACGCAGAGTCGGATCAAAG
CTAAAAGAGGCAGCGAGCCAGCCATCAGATATCGTCATCCTCTTTGATTTAAAGCATTTCTTACTGTTTT
TATTTTCTCTGATATTTTCTCTCTCTCCGAACAAAG
Sequence ID: 105
Sequence Length: 6250
Sequence Type: DNA
Organism: Poplar sp.
ATCAATTGCCTTAGTCAATTGGCTAATATGATAGATAGTAAAATCTGTCACAGAAAATTTTTTTACACAA
CATCTTCACTCAATAAAGGCTGTCTATGGATTTTGGAGAAAAAATGATAATGAATGGTTCATATTGATTC
TGTTATCATATATTTTTCTTTTGGTTCTCCATCATAATTTGTGCCCCACCCCCCCCATTCAGCTTCGGCT
TGCGGCGGAGAAGTATGAAGAGGCAGAGGCAAGAGCTCGGCAGCTCGAGAGACAGGTACTTTTCTTGTGG
TTGACAAGAAAACTATTGCGTGAATGTGTCTGCAGATTCTGATGGAATCTGTTCCATTCTAGCTATTAGG
AAATTGAAAGCATGTTATATTCATGTTGGTGAAGTTCAACTGTGCAGATTTTTGCTGCAGCATGTATTTG
AATTTCAGCCCTTAGTTGATCTTTGATTCCATATTTTTTGTAACCACTGTCTGTTTCCTTTGCTTTCTCC
TCCTATTCTTGGTTGCTAGCATTTAAACATTTATGCTGGCAAGAGCATTTTGACATTCCACACATCCCCC
TCGAGCCAGTCTCATGCTATGTTTTTCTTCAGGTTGCTACTCTAGGTGAAGGTGTAACCTTAGAAGCCAA
ACTTTTAAGCAGGCAAGCATCAATATTGAAATTTTTATCCCTCACGCCTCATTGTATTTTAACTTTCTAA
TTATCATTATTTGTTTCGTTTCACCAGAAAGGAAGCAGCTCTGCTACAAAGGGAGGTTCGTATCTTTAGC
AAACATTTCATGTTAGTTTGGAACTTCAATATGCTTGTGTTTTTTGTAGTTATCACAATGCACTAAATGC
CAGAAGGTAGATAGAAAAACCCCCTAGAGAAAAACAAGCTAGCATGTTTTCTTGCATGTATGCATTACTA
GTACAGATGTCTGCTGCTATTATTGGTACTTATGAAGAATATGAAGCATGAGCACGATTTTACTGCAGGC
TGCTTTGAAAGCAGCAGAACAAACTAGTAAACCTGAAGAGGCCCTTCGGTTGGAAGCTGAGGTATCGACA
AATTTTATGTTGTATGTTTGTCCCTCTTCTTTTTAACCATTCTTGAAAGATTCATGACAAGGTGTTGTCA
GTATAATTCACATTTCCAAGATCATGATTTAGGTTGCTAAGGATGAGGCCGCATCTGCGATTGAGCAACT
CCGTGAAGTTCAGTCTGAAGCTAAGTCACTCCAAAATATGACACAGCGAATGATACTGACTCAGGAAGAG
ATGGTTTCTCTTCAACCTTGCTTCTTATCATCTCATTGTTTATTCTTCTGTACTCCTTTATGTCCTAGTA
ATGTGATTTGCAGTTTTCATCTTAGGAAGAAGTTGTTCTAAAGAGGTGCTGGCTTGCACGATATTGGATC
TTGTGTGTTAAACATGGTAAAACCATTTGAGCTTTTGGCCTTTTGCTATTCCCATGCTTTTGAATTTTTA
TCTAAATCTAAACTTCTGATTCTGCAGACTTTAAGTAAGAAACCTTTCTAAAACCAACTTGTGGATTGCA
ATTTTTTGCAGGTATTCTCGCAGAGATTGCTGGAGCAAGATATGAGTACTGGTCATCCTTTGCTCCTCTA
CCTGTTGAAGTTGTATTATCCGCTGGACAGAGAGCTAAAGATGAGAATTCATCTGGTGAGGATTTGTTCA
AATATATCTATATATTTGTTTCAAATGGTCAGTTTACTCAATAAATTCTGCTTGCTTCTTCTGGAGCAGT
AAATGATGACGCAGAAGAAAGAGAGAGAGTTCTAAAAGAAGGAAGTGAACTTTCTGGAGATGGAAATATT
GAGAGCATGCTTCTAGTTGAGAAAGGCCTGCGCGAACTGGCTTCACTAAAGGTTTCTGACGCACCCTTGG
AAGTTTCCTTGCTCATATTTTGTCGAATAGCAGTTGTTTTCTCTGTTGCTTTTGCTATAAGCTCTAGTCT
TAGCTAAGCAAAATCATGTTAAAATAATGAATGCATATGGTTCGATAATTGAAGTTATCACTCGTCAATT
TCCTATCAGGTTCTGTCATGCTTTATGTCAAAGTGATCATCCATATATCCAAAGGTTGTAAAATGACACT
AAAAGCAGCATTGCTTGATTTCTTATGGCCTCAAATACAACTTAATAACCAGTGGAACCCCTTTAGGCTT
TTGTTCATGAAATGTACAGCTCTTAATCAAAGATTGCTTTGGATAGTTAACTGTTACTGTCATTTGGGCA
TCAAGTATCATTTCATGAAAACAAGTTTGCTTTAATCCAGGTTATTTTGCACAATCTCCTGTTGCTCTAC
ATGGTTTCTAGTAGGAAAACATGTTTAAGAAAACATTTCTGAATTAATCAAGGAAATAATCTGGCTCATC
TCTAGACAAGTTGCTATTCAAAGTGCTGGTTTCTTAGCAGCTCTTGAATCTTCCTTTTCCTTTCTTTTCA
AGGTAGGGGAAGCAGTAGCTCTTGTCATGGCTCAGCAACGTCGGACGAATTTTATGAAATCAGGTATGAA
TTAATTATGGCTTGCAAATTGAATGATTTTCTGCATGAAAATCTATATTATTTTAGATTTTTTTTTCCTG
GTTTTTCGAGTTCTTGTTTTTTATATTTTAAGATTAATTTATATCTCATTGCTGATACATGACTCAATCT
GATTGCTTGATGTTCACTTGTTTTCTTTGATTTCTCATTATAATGGCCGAAGATGAAATTAAACTAGCCG
GCGATGGAAATCTTGAAGCATTTGGTAAGTCCATTGCTGAAAACATGATCATGAAAGAAGTTTTTCTCTT
ACAAAGGCTTGTAGTCTTGGCACTGCTATTGAGTAATATATTGTATTATACTGTTGTCTAAAATTAATCA
TAAGAATAAATTCTTTCAGAATTGAGCCAGGAGGAGTCCGAAGACGTGCGATTCAAGCAGGTATGATAGC
ATCTGCATACAAATTAGCATGTTTCAATTGTTAAAATATCAGAGAAAGTTAAGTTGGAGCAAAGTTAGTT
GACCTGAGCAATATTTTTTTTGGTTTTCTTGTTTTGCTGGACCAACATGTGATGTTCAGATCTCTGAAAT
GATGATAATAAGTGCGAGGGCTTAGTGCTTGGTAGCGTTTGGATAGACGGAGATAGTAGAGACAGAGAAG
ACATGTCTCTGTATACCTAAGGACAAATTTGCACCGAAACTATTTCAGATTTCTCTGTCTCTCCAACCAT
CCTTTTGTATTTGTCCCGTTTGTCATGAGATACTAACCGAATGCAATCTAATGATCTCGAGACAATCTAA
TAAACGATAATACTAACATAATTCTGCTGCCAAACATGTGAAATGAATGTAATCTCATCAAATTCATACT
TCCATTGTATGTTCTTTAGCCCTTCACCCTTCTGATTCGGTTTAGGGAAGAACTTGTGTTCATGAGTCCT
TTCTCAAATACTCATTCGGTATCCACACACAGGCCTGGCTTACATATTTCTGGAGAAGAGCCAAAAACCA
TGGTCTGGAACCCGACATAGCAGAAGAACGTTTGCAATTCTGGATCAATCACAGCAGTCGGTCTTCCTCG
TCACACGATGCAGTCGATGGTAATCTCCCTCCCTCTCTCCTTATCCGACACACACAGGCACGCACACACG
CACATGTTAAACACAATCATCTTCCTTCTCCACATCAAGTAGTCTGCTAGTTTCAATCCTGAACACGAAT
GTGTGCATTCTCATTCCCGCAATCTTTCTTTTAATTTACTTGGTTATGATTCACTCCAATGCGCAGTTGA
GCGCGGACTCATGGAGCTCAGGAAATTAGGCATTGAGAACCAGCTATGGCAAGCATCTCGAAGAGGGCTC
GAGGTTGACTCCAACTCCAAGGCCAATCTTGAGTCCGACTTCTGATGTAACGTTGAAGCTGTTGTCTGAC
ACAATAATCATTGCAATTGGGAAATCTCCTCTACATGTTCATATCAGCTAGCTGATAATTTTCTTTAATG
CTTCATATTGGGAGCTATTCATCTCCAACTACCTTATTCTTGGCTCCATACTTCCACTCATGTAAATTTT
CTATGGCAACGAGACATAACTGTAACCAAATATATTTAATTTGATACGCACAACGTATTCTATGGCAGCC
ACACAAATTCAATTGCCCTGAACAAATTTTGTCATAATTCAAAATGCAACCATGAACTGGGAGGCTTGGA
CCTGTGGTTAAACAGAATGCATTCTTTTTAGGGTTCATGACTCAAATCAAGTCAAAATGTGTAAAATTCT
TAAAAACAATGTTTTTTTTTAATTTTTTAAATTATTTTGACCATCAACATATAAAAATATTTTAAAAATA
TAAAAAAATAATTTTAAACAAAAAAAATTCAAATTTTAATGGAACGTGGTTCGAATCGCATTCCCAAACA
TATGAACGGATCTCATTACAAAAAAACTATATTATATGGTGGTAATTTGTACAAGACTAATTAGTATGCT
AATGTTATTAAATTTGATTAGATTAATGATTCAATTTAAAACTCGAGTAATAGATTAAAAAGCTTAATCA
AGGATAATTTAAAATAATATCATTTTAAAATAAAAAAATTTAAAATAACAAAAAAATCTAAATTAAATTT
TAATCGATCAATCAAGTTTGACTATAAATTTCACCTTATCTAATTTAAAATCCAGGCATGACTAAAGAGG
GTATTGAATTGACTTGCCGGGTTTAGAGTATGTTTGAAAGTGTGATTGCAATTGTTTTTTAATGCAAATG
CATCAAAATAATATTTTTTTTATTTTTAAAAATTATTTTTGACATCATCATATCAAAATAATTTTAAAAT
AAAAAAATATATTAATTTGAAATAAAAAAAAATAAAAAAATTTTAATTTTTTTTTAAAACATAAAACAAC
AATAAGCTTAATATCATTGCATAAAGCTCGTGCATGAGCTGGGTCATCGAAATCGACAGCCACTAGATAT
GGTAAATGATAGTCGGGCCACCTTATCCTTGTTTTCGATAAGATATTCATGCAAAAATCATTCTGCAACC
GATATATATATATATATATATATATATATATATATATATATAATAATAATAAGAATTAACAGAGAAAAAA
CATAGATAATGGAGAGACATGTTCAAGATGATGTCCAATGGACCGACTAAAACCCAGTAGGCCCATCACA
TCCCAGGTGTCACCTTACTGTAAATAAATCCATTAATTTTACTGTCAGCAATGCATTCATTTTCACCACA
CTTGAAAATGACCTAGAGGGGGCCGTTGGGGTAAAAACTAAAAGAAAGGAAGTCTTACCAGACTCTCATT
TGTCCAGTAATGAACCACTCCCTTCTATAAAAAAGGTAAAAAACACCTATCTCTTCACTCTTCTTCACTC
TCAAACTCAACAAACAAACATACTGTCTCTCCCTCTCTCTCTAAGGGGTGCTCTGAGGTAAAGCTTCAAC
CTTTCCACTCTCTTCTTTGATCACATTCAAGGGTCTTAGTTATCATCTCGTGATCATTTGATCTTTGATC
AGAATCTTGTGTTTTTTTTTTGGCTTTGTTATGCTTTGACTTTTTAAAGATCAGCCTTTTATGATTTGTT
TGGTGTGATTAACTAGTAGTAGCTTGATGATCATACTAGTGTTATCTCGTTTTCTTATCATATATGCTCT
CTTTATTCACAAAGTGTTGAGTCGGTACTTGGATTTGGTGTTTTTTGTTGTTACTGCTTGTGGGTTTTGT
GTTTTTTTTTTCACAGATCTCTCTCAAGTTTTGTTCTTTTAATAATATATAAAATTATCTGTACCTTGTT
TGGGTTTTGTAGATAGAATAGACAAAAGGGCAGGAGAGAAAAATAAAGTTTTTTAAATTAGGAATTGGGT
CGATAAATGGGTATTTTGTATAAATTAGAACTACTTAATGATACTGAAGATGTGCTAATTTGTTTTGGAA
TCATCAACTTTGCGTACTTGTAGCTTTGTTTTGAGTAGCTTTGTGGGTTCTTTAGCTTCGATTAGAGTGA
TTTAGTAGGTATGATTGATGTGTACTCTTGCGCATAGAAGTTTAAATTTTAATTTGGCTGTTTTGAGCTG
CGAATCTTTTGGTGGGCGTTCATGTGCAATGTGCCTTTTATTTTTTTGTTCCCTTGTTGTATTCGGTCAG
GATGTGGTGTTTGTGTGGTTTTTTTTAAAGAATTTAGGTGATTGTAGATGTGCAGTCCTAACTCTTGTCT
CTTTTAGATCACATAGAACA
Sequence ID: 106
Sequence Length: 4006
Sequence Type: DNA
Organism: Poplar sp.
TGTCATTATCAGCTCCATAAATAACAAATTACTTGAGGTGTCTATCATATGATATGATGATTAAACAAAG
AAACAAGTAAAAAAAATGGTAGATTCTCACCTAATCATTTAATATCTTGGGATAAATTTCAGTGAACTGT
TTTCCCTTGTAGTTTTTATTTTATAATAATATTGTTACTAAGGTCGTTTTTGACACCGTGATACAGCCAA
TAATTATTGACTAGCTAGCAAAGAAGAGGACTTGGTATGTTATATATTTTTATTGTGTTTTTCTACAACT
AATCAGAAGAAATCAATTACTAATTTGACAATAAAAGCATGCAACCAAAAACGAAGTTTGAAAATTAAAA
AAATAAAATTATGTGGATTCGTGTTCCTTTTCCCCAAATGTTGCAAATGTGTTTTTTTGGTATAACAAAA
TCAGATACATACTAGATAATATATTTTTAAGATATTAAGATGGTGATATATTTGATGACATGTAAATATT
TTTTATATATATTAAAGCTTTGACATATTAGATTGATTCAGAAAAATTTGGATTAATCAGAAAATTCATG
ACCCTGATCATGAGACAGTGACGGAGCTAAGGGGAGGCAAGCAGGAATCCGGGCCCTGAAAAACATTTTA
GATTTTTTTATTATTTAGTATTTAAATATAACAAGTAATATTTTTTTATTTTAAATAGATGGGCTTTCTA
ATAATAAATTATTATTTCAATAGTTATCTTTGACTTTAAAATTATCTCTCCGTGAAAATGAAAAAAAAAG
AGAGAAATAATGTTTCTTTACCGTACCGTCTCTCTCTTTTTTTTCTTTGGAGTCCTAACCAAAAAGTATA
ATAATACAAAGCGGAAAGGTTAAAGCGAAGCTGTCTATTAATTGTTCATCAAAACAAGGTAACTAGTGAC
TCAAATTTAATTTTTTTTTAATGTTTGTTGAGAATTTGATGAGGTTTTTTTTATATATATATAATTATGT
TATTTTTTTTTTTCCAGACCTACTAGTTTTACCATTTTTTTTTATTTCTTGTTATATTGAATGTCTTTTT
ATGAGTTTGTTTCAATTATGCTTTGATTTTTTATGTTTTGTTTTTAGCAAAGCCACCCAAATTATCAATT
TTTTCTTACGTGCATCTTACTATTACCAGGTCCAATATACAATTAGACACGATCTTTCTAGCCAAATTGA
GTTCTTTGAAGAGTACATACCTGGCTTTGAAAAGCACATACCCAGATGAAATTAACTTTATAACTTTGAC
TTAATTGGGTATTGTTTTAAACCTCTTACTTTATACAAAGATCATCACATGTTTGTGGTAAGTTCTTTGA
ATAAAACTAAATCATGCAGGGGTTTTTTTACAATTCATGTACAGTAAATTAAAAAAATTATATCTTGTTA
TATTAGTTTTGACCCCCTGTTAAATAATCCAAGCTTCACCCTTGCATGACACCGTGATAACATCATATAT
ATAGAAAGCAAATCAAATCAAAACAAAATTAGAAAGCCCAATTCCAAACAGCAAGATGATGAAATGCGAA
AGTAAAAAAAAAAAAAGTATATTCAATTTTAGAAAAGGATCCAAAAAAGCAACTCGAGTAAAGTTAGGTA
AATATGTCAAACCAACAACCCGAGTCATGAGAACAAAATTACTTCATAGAAAACAAATAAAAAAATAATA
AGGCTACTTCTTAATCAATCTAACGTTAAAGAAAGCAATTGAAAAAAAAAATTGTTTTAAAAAAAAAAAA
AAACAACCCGAGTCAACATTTCGAATCCACAACTAAAGTCATGAAACCGAGATAACTATATAAAAATAAA
ATTGAGAAAATTACATTTTTTTAAGTTAAAAGATAAAAAAAATTCACATAATCCAAAAATAATAATTTTA
TTTGTGTATGATTTTTTAAAAAAATAAACTGGTTGATTTTATTTAAGTTAAGGTTTTTTTTTTTAAAAAA
AAGAAGGAACGCCTTTTTAAAAGGAGCACATGAGTTTAAGCTTGATGGACTAGCTCTTTTACCCAAAATC
AAAGGCATGTTCCTCTAGGCCAGCCCGTTAACTTATTTTTTTTTTACTTGGTTGGATGAATAATACGTTG
TATGCTTGTCTTTTTTTTATTAAAAAAAATAACAAATAATGCATCATTTAATTTGACAAATAATATGTCA
CCTTTAGTTCCACCACCAACAACATTAGGTGGCTGAAAAGTCGAGCTAATGATAAATTTTTCTGGAAAAA
AAATTCTTTTTAAATATTTATTTACTGAAAAACACATTAAAATAAACTCATTTATAGGCTCAAATTAGCA
AAAAATGAGTTTAAAAACCAAGAATTAACCCCAGTTCAAAAACACTTCTCGATCCCATATCTATTATTAG
TCAAGATGAAGGCTAAATGACACTTCATTAAAAAGAGCATGTTGCACTAAAATCAATTATTTTCGCGGTT
AAAATCTATATCAACCGATATTTTTTCTTTTCTCAATTAGAATCCAGCAACATTATCTCTCCTATTTTAA
TCTTGAAAGCTAAAAAGTAAAACAAAAAAAAAAACAAAGATTTAGATCAAAAGTTAACTTTCAAGAAATA
AAGGGATTGAAAATGTTTTTATTGAAAAGTATAGAGACTAAAATGAACTTTCATGACATTTTTTAAAGAG
CAATTTTTTTTCATTTTGGTCCCTTTTGTTTAAATGTCATCCTTCCATAACAAATTAGAAACAATTGGCC
ACTACTTTTATAATTGAAGGACAAAATTAAAAAAAAAAGTTTGATAACCAAAAATGAAATAAAAATGCAA
AAATTTCACTACTCCAATCCCCATAGTAAATTGTGTGAGGGAGCACAATAAATTTTGCTATAATAAAATG
ACCACACCTCTTAATTTTTTGCTAATATAATATAATGTTATATAGAAATCCTAAAATAAAATTTTCAAAG
TGTGATAAAAAAACTAATTGGTTTGAAGATAACATCAAAACTCATAAAAAAAAAATGATTGTCTCGCCAG
ACTCTGATTTGTCTAGTAATGAATCACATTTTTTTATAGAAGCGGCCAGAAACATCTATCCACTCACTCT
TCACTCTCTAACACGACAAACAAGCATACTCTCTCTTTCTCTCTCTACTCTCGAAGGGGCGCTCTGAGGT
AAAGCTTCAATCTTTCCATTTTCTCCTTTACTTTTTCTTTTGATCAAAATCGAGGGTCTTAGTTATTTTC
TCATGATCGTTTGATCTTGATAAGAATCTTTTTTGTTTTTTTTTTTAGTTTTATTATGCTTTGACTTCTT
AAAGATTAGCCTTTTATGATTTGTCTGATGTGATAAAATCATATGGCTATTACGAAGTTGTTGGCTATTA
TGTGTTCTTTTTATTCTTAAAGTGTCAAGTCTATACTCAGATTTGGTTTTTTTGGTTGTTTCTGCTTGTG
GGTTGTGTGATCTTTTCACAGATCTTTCTTGGGTTTTGTTTTTTAATCAAGGCAAGCTACCCAGGTATTG
TTAGACCTCAATTTTAGTGTTTTCTTTAAGACATAACATTTACTTGATCTTGTTTAGAACTTGTAGATAG
AATAGTCAAAGGGCACGAGAGAAAATAAAGCTGCGTAAATTAGGAGCCGGATCGAAAAATGGGTATTTTG
TGTAAATTAGAGATGCTTAATGATACTGAAACTACACTGATTTCTTATGGGATCCTCAAATATGTGCATT
TATAGATTTATTTGATTAGCTTTGTGGGTCCTATATCTTTAATTAGTGAGATTTATTTGTTATAATTTAT
ATGTGCTTCTGCACTTATCTGAATTTTAGATTTTAAATTGGCTGTTTTGAGCGTAGTCCTTTCTGTGTGT
CTGTGGGTGCAATGTGCCTTTTGTTTTTTCTTTCTCTTGTTATATTTGGTCAGGTGCTATTCTTGCGAGA
TGCAGAGTTTGTGTTGATGTTTTTAAGAATTTAGGTTATTGTAGATGTCCAGTTTTAACACTTACTCTTT
TAGATCACATAGAACA
Sequence ID: 107
Sequence Length: 3000
Sequence Type: DNA
Organism: Poplar sp.
ATAATATTTTACCTAATTTTATTGCACGCTCAAAAAATCATGAAAACTATAGTTCTTGTCGAATGAATTT
TGTATGTAATGAAATTGTAATTTTTTTTAAAAAAAATTGAGTTTTACTCGAAAAACTAGTATTTAATATT
ATTTATAACACTACTTAAATTAGAAGGATATCGCATGATGACGTAGCATTTGTGAAATTTGATCGTCATT
CCAATTATATTCTTGCCGGGTCCAACCCAGTTAAAAAAATTCAGTTTTTATTTTTATTTTCATTGTGTTT
TATTCAAAAAATTAGAAGGATATCGTTTGATGACGTAACAAAAAATTCAGTTTTTGTTGTTGCATGCTTA
AGAAACCATGAAAAATATAGTCATTGTTTGATAGATTTTGTATGTGATGACATTGCATATAGTTTAATGG
AATAATAAAAAATATTTGATATCAATATTATTTATTTCATGATGTAATAATAGTAGTTAAATCTTCAATA
TTTAAATTAAAAATATTTTTTAATTATTTTATAACCTCAATTTGAAAAGCATTTTTTTAACCAAACACAT
TAAACTACTTTTAGTTCAACCTCAATTTCAACCACAATTTTAACCAAGCACCTATTTTTTCAAACCAACC
TTAACTAAAAATACTTTTTATAAAACAATTTTTTTCAAACCACAATCACAATAGCAACCGTAATACCAAA
CATACTCCGATGAGTGGTAGCGATTCCATTAAGTTGTCACGTGTATCATGGTTCTCGACGGCCTGGTGGT
GCTTGGGTTTTTTCTTCCCCCCTCATTCTCTTTTTATGGCATTCAGGTGTGCTTTGACCAAGCAAAAAAA
AAACGTCCTCTTGTTTTATTTTTATTTTAGTCATACTTCTCATTCTTTTGATTAGTCCTTTATCGTTTTA
ATTTTTTATTATTATTATTTTATTTATTTAAATTAATTTTATTTGGTTTTTTTATTGGATTTGACTTTCA
TTTTTTTAGTTGTTATTTTTTTCTTTTCAATTTCAACCCTTGAGATTTAATTTGATTTGTTTTTTGCATT
ATATTTGATTATTATTATTTTAATAGTTATTTATTTTTGAATTTCATCTCTTGAGATTTTATTTTTATAC
CAGATTTAGTCATCGTTATTTTAACTATTTTTTTTTATATAGTTTTTCTAATTAACTTTTGTTTTTTCAT
TTTATTCCTCAATATTTTTTATTTCTAATTTTGTTCTCATTCCTTTAACATTTTTTTTTCATTTTTTTTA
ACTATTTTTTTTTAATTTTCATTCATCAATATCTCACTGATTTGTAGTCTGGATCGTAATATTTTTTTTA
ATATAGAGATCTCGGTCTCATGCCCAAGGGACAAGTGTAAGATATTAACCCGGGTCGATATTGGTTTTTT
ATTACCCTTTTGTTTTAAACTATTTTTTTTCTCAAATCCACTCTTCAACATTGCATTTATTGAGGATTTG
GTTCTTTTATTTTTCTTATTTTGCTTTTTGTGAAGATAGTTCGACCTCATGATCTAAATCACAAGTTTAT
TGTGTTGGTTCAGATTGGTTCGAGCAATATGCTTGAGTCTCATTTTATAGTCCATTCATATATTTCATGA
TGAGAATCTAATTAGAATATTATTTGAGCTATTTTTATTTTAAAAAACTTATTTAATTCAAGATTTTTTT
ACCCTGATTTTTAAAATTAACCAAACATTTCATCATTTGTTTGTTTTTTATATATTTTTTTACATTAGAC
AATAAATCCTCCTTCTTTTTTTGTTGTACTTTAAAAAAAATTAAACTTTTTTATTTTCCAAGGTCGCACA
CCGCCTCTATTTTTTGTCTTTTAAATAAGCAAATTGTTATTTGGTATGAAAATATCTCATGCATTTGGTT
TGCATGTGTTTACATGAAAAATGGTTTTTTATATAAAAAATTGTTATATCTAGTAGGATTATGAATTCAG
ATTGGTGGTATAGCTGGTTAACTTATTTTTTTTTAGATTGATTTTGTTTTTATTTCTAGTTTTTATATTC
TTTTTTCTAGTATTTGATGGGAGAAAAAAGCAGTATAAACCTGCACAATCATTTTTTTAAATAAAAAATT
TGCTATAAAAAACGTGTGGAATAAGCCATCACATTTTTTTTAAAAAAAAAGATAATATAACTAGTACAGT
ATAAGTTCAACTTTAACGTTGCAAGTATCGGTCTACGTATCCCCTACATTATACAAATCAAACACAGTGC
ACTCGCAACAACTATAGTTGTCTGTAAAAAAAAACCTTTATAAAGAATTTACAGGTTATTAGAAAATTTC
AAGATTTTGTAGAAATAAAATAATTTCAATATTTTTTTTTTAAATTTTAATACTTGAAAAAACACGAATG
AATGATACAAATAATTAAAATTATAAATTTTAATAAATTTGCAAATCATATTCAGAGCGGCACATTAAAA
ATTAATCTTAGTAACATTATCATCTCTCCATCTCCCTCGTATAATTGATTGGAAGATTTTTTCTTATAGA
ATGAAAATAATCTAAGTAAATGATAATTTTTTAATAATTTAAATAAAATAAATTTTAAAACTATTTTTAA
AAAATTCAACTAACAAACCAAACCAATTCAAGACACCTCAATCACGATTCCTATCATGTTAACATAATGA
GTGGTCCAAAAATTGTTAGATGGCCCAATCCCTGAAATACACCGTCCTATCCCTCAGCTAACCATGACGT
ATTAATCCATGGGCCTATAAGAGCATCACAAAACTGCCAAGCTGTTTCGTTAATCCTAACATCCTTTTCA
CTTCAATTTCCAAAACCACCCTTATCAAATCCATCTCCTCCAAGCATATTCGAAACAGATTCGAGGCTAA
AACAGTAAATAAAATAACCACAGATTTATGGATCCATCACCATAAGAACTCCACAAATATCAAACAAAAA
CCCGAAACTGAAAACAAAAACAAAAACTAAAAATCAAAACCCGTCACTCCACAATCTACA
Sequence ID: 108
Sequence Length: 3047
Sequence Type: DNA
Organism: Poplar sp.
CTAACTTTCTCGACTTGTCGTTTTGTAATGACAGATAGAGAGGAGGTATAATTTTTCTTTTAGATTTTTT
TTTATTTTTAATATCAGCATGTAAAAATTATAAAAAAAATATATTCTTTTAAGCAAAAAAATTAATTTAA
AAATTGAATTAAGAGATTGACAATTGAAACGTTGGTGGGCAGTGGGCAGTGGGCAGTGGGCACTAGCACT
CCGTTATATATACACATATTGATAAATAGATAGATGAGAGGTAATTTTAAGGGGTGTAATTTAACTGTTT
AAATTTTAAATTTGTTTTTTAGAAATTATTAGTTCGAGTTTTATAGATTTCAGAGCTATTTGAGGCTTAC
ATGGTCATTAACTTCAGAATCCATGAGATTAATTAAAATACACGAATATTGACTTGAATATTCATATTAA
TAATAAAAAAAAAACACTAGAGTTTGCCGAGAAAATATTGCTAGAATCTCTGCATGTGTGACCACAGACA
GTGAGCCAGGCCTGAAAAAATCATTAAACCATCCCATACCGTCGTCCACGCCACCTATATAACAATCTTC
AAGAATTTGGTACCATAGAAACCTAACACAGCAAACAAGTTTGCGAGACAGTGAAGATTGACTCTTGGGT
AGAACTTAGAATCTTGTCTAGAAAATGAGCTGGTGGTGGGCAGGAGCTATTGGAGCTGCCAAGGTAATTA
AGATTAAACCAACCCCATCTACAAATCTTCAGCACTTTAATATTTGAACTTGATATTTATGTTTTAACTT
AACATTAATTTCATCTGTTGGCAAATGTTTTGCAGAAGCAATCTGAAAATGGAGACGCATCACGAGGACA
CCAGAGCGTGGCTTTAGTGGTAGGAGTAACTGGCATTGTTGGCAACAGCTTGGCTGAAATCCTACCACTC
TCCGACACGCCTGGTGGTCCGTGGAAAGTCTACGGAGTGGCTCGTCGTCCACGGCCAAACTGGAACCTAG
ACCACCCAGTTGAATACATCCAATGTGACATCTCTAACACGGCCGACACTCAAGCAAAACTCGCCCAGCT
AACTGATGTTACTCACATCTTTTATGTCACATGGGCCCTCCGATTCACGGAGGCCGAGAACATCGAAGCT
AATAACCTCATGTTCCGCAATGTCCTTCAGGCTGTTATCCCCAACGCCCCCAATCTCAAACATGTTTGCC
TCCAAACTGGTCTTAAACACTATGTTGGTCCATTCGAGTTGGTAGGTAAGATCGAACCACATGACACTCC
TTACACAGAAGATCTGCCCAGATTAAAGGCACCCAACTTTTACTACGATTTAGAAGATATTTTAGCTGAG
GAAGTGGCAACGAAAGAGGGAGTGACTTGGTCTGTGCACAGGCCGCATACAATTTTCGGGTTTTCTCCAT
ATAGTTTGATGAACATGATGGGCACTCTCTCTGTTTACGCAGCTATATGTAAACATGAAGGGATGCCTTT
ACTATTCCCCGGGACTGAATCTGTTTGGAATGCTTATTCCATCGCCTCTGATGCAGATCTGATTGCTGAG
CAGGAAATTTGGGCAGCTGTGGATCCTAATGCACAAAATGAAGCGTTTAACATCCACAATGGAGATGTTT
TTAAGTGGAAGCATTTGTGGAAGGTTTTGGCTGAACAGTTTGGGATAGAAAAATATGGGTTGCCTGAGAG
TGGGAAGACAGTGAGCTTGACGGAGTTGATGAAGGATAAAGGAGCAGTGTGGGACAAAATTGTGAAGGAC
AATCAGCTGTTGCCTAACAAGTTGGAGGAGGTCGGGGTGTGGTGGTTTGCCGATTTTGTGTTGGGTGCAG
AGTCCATTATTTCGTGTATGAATAAGAGCAAGGAGCATGGGTTTCTAGGGTTTAGAAATTCCAAGAATTC
CTTGATTTCGTGGGTGGATAAGTTGAAGGCTCACAAGATCGTGCCTTGAGTTTGGTGGATTTAGGTTTGC
TGGTCTTTTTGCTGCTGTCATGTTGTTTTAAAAAATAATTTGGAGGATTGGAGGATTTTCTTATTGTTGA
TGATTTCTATTAAAGCATATCTGAATTCTGAAGAAAAAATATTTGCTGGAGGTCTATTTCTCTTTGTTCT
TAATTTTATTAGAAAAAACTCCGACTGGGCTGGAATTAATATTAGTTAACAACTTGTCTTGGCTGCCTCC
CTTCGAGGTCAGGGTCCAGAGCCCACAAGACAGGTAACTTAAACTTACAACTCTTCCTACAATACAATGC
GAGTCGAATTAAAAAAAAAAGAATAAAAAAGCATGTATCAAGAAATATAATTATTTGATATTTTCTATTT
TAAATTAAATTATGTAAAAATTATTTTAATATGACTCAATCAACCATAGCTAAATTGTTCCCCCTAGTTA
ACCAAAAAAGTCTGAAGATATGTTAAATCAAATAAAAAATTGAGACAAAAACTCGATTAATTATACCAAT
TTTGTATGCCATGGGTTACTAAGTGCATGCAACAAAACCCACTGAGTCATAAAATGCACAAGTTGATGGT
GTTGTTGGTGCAGTCATCAGGGACTAACTTGATACCAATTCGATAGATGGAGGGTGTATTTGTAGATTAC
AATTTTTTGTTTGCCTGATTTTGTATTATGGAAAGGTAGAAAATTGGTATTTTTGCTTCTAATCATCCGA
GTCAGCAGAGTGTCGTGCACAAGTTGTCGAATGAAAGTTTGGTTGATATGATATAGAAAACAAAAGGAAA
AAAATTACAATCAAAGGGATTTTTTTAACAGTAAAAACTCATTTCCTTTAGTTTTTTTTTTTTAATTTAA
CAAGCTCAATCAAATTACATACTAATCCTCTAAATTAATGTTAAATTTAATTAAAATTAAAAATCGAATC
ACGTTTCTTATCATTACTTTTGTCGGATAATCCCGACAGGCAAGCTTTACTAGAAAACTCTATCTAGTTG
TCACGTATGTCATGGCTCGCGTCACATAGCTCTTGTTTTCACGCGAGAACCAATTTTAATAGGGCGATTC
TGAATCAAAACATATCCAGAAGCAGCAGCAAGTTAAC
Sequence ID: 109
Sequence Length: 3127
Sequence Type: DNA
Organism: Poplar sp.
CGTGGACATGTGCCAAACACAAAAGACCACTCCACAAAAAAAGTGGATTAGTGTGTAATTCATAAATAAA
TATCTTGGAAATGCAATCATCGGCAACACTAAACCGACAATCTGTCCACTCAAGTAACGCATCCACGACG
TCAAAGAGCGATTTATCACCTTAAACTATTGCCTCTCACCACACATGAACCGTACCACAAACCTGCTAGT
CGCAAATCTCGAGATACCCTTCCCTTTCTCAGTTTCTTGCCGACGAAACCGCCCTGAGGTCCCACCCAAC
TCGGGGTACTTTTTCCGATGACGAGGAACGACGCCCAGACCCTTCGTACGGTTAAATCCTGAATACTTAC
ATAGGATTCTTATAATTACTGTTTAAAAAATGGATTACGCTCCGTGGTTGGCGGTTTGTCTTCAGATTTG
TGCAGTTAAATTTTTTAAATTAATATTTTTTATTTTTTTCATTTAAAACTAAAAAATATATATTATTTTA
ATATATTTTTTTAAAAAAATACTATGAAAAATACTTTATACCATGATTTTAACCACCTCCAGACTATATA
TATGAAAATACTTGATTAATTAATACACATTTTAATTTTATATTGATTTTACTAGAAAATATTTTTTAAA
ATTTTAATTAAAAAAATATAATTTATAAATTTATTTTTAAAAATTATAATTATAAAAAAATACCACGGTA
TCAACTAAGGCAGGTAATCCAAAAAAGGCAAGGCTACAAAAAAGTCGGATTTTATAGGTCAAGATAGATT
TTTATACCCTAACCCTGCCAATTAACTAGTTTGTAAGTGATTCTGCTGGTCACTGAGGTAGAAATGCTAC
CGAATAAGAGGCCCAGTGCACATGACCCCTCTGAAAGTTTAAAATGATGACCAAAATTTAATAGTTATTT
TATATAAATTTTATTTTTTATAAAAAGTCCTTTTTAGTAAAAAAAATAGTATATTTTAGTTAAAAATCTA
TAATTATTGTATATATATATTTTTTATGTGTAAATGATTTTTGTATCTTAATATACCCCCCTGCTTCTAT
AACCTTGAATTCCCAGTTCCTCCCGTGTAGATCTTTCAAATCATGAATACTAGATATCTATACAAATTGA
TATGTTAATTTAATTAATTTTATAAAATCCAAGCCCGCCATTCTTTACTTCTTCTACTAATTTGTTTATA
AAATATATGTTTCAATATCTCTTGATAACTCAGTAATCCAATAATTAATCTAATTAGTAAGTTAATAGTT
CTAGACCAAAATGAATTAAACGAATAGATAATTTTGTGTGATAAGAATATATTTTCCCATAAATCTTCTA
ATCTTAATATAAATAATCCGTTTCTAAAATTAAATTTAGTAGCCATTCCATGTATATTACTAGAGAAAAC
AATAAAATATCTGAGGTTGCTTTGCTGCTTATTTGAACAACGAACACCTCTCCTGTTTATTTCTTTCACT
CTTGCCTTTTTTTTAATCTCACCCTCTCGTATTTTTTTTATTTGATCATTTTCACAATAAATTGATTTGC
TTTTCAGTTTTATAATTTGTTTTACTTTACTTTACATGGAGCTCTTGACATGTTGAAAAAAACTTTTAAT
GCTCGGTTGATAATCAACTAGACACATTTAAAATAAATGAATCTTTATAAGGCAAATTTGAATCTAAAAT
AAATGTTCAATCATTTTTTTTTTGAAACCAGTGACTAGATTTAACGAACTTGAAAAAAAAATTAATCATA
TGTAGCACATGCACCAATAACTTAGAAATGACATAATATTGTGACGACAATTGCAACGACTACCAAGCTC
GAGATGCGCCCTTACATGGTGCCAACAAAATCATCACGGTGAGGAGTTTATTATCTAATGGCTCATTGAG
ACAAGGGCTCTCCGGTGTTTTTTCTTTTTTCTTTTTTTTTCCTCTCTTGTTGAGTTTTGCGTTTAACTCT
GTTGTTTTTTATATATATATATATATATATGTTAAATTTGATTTTTATTCATTTGATTGTTATTTTTAAC
AATCAAATGAATAAAAATCATGTAAGGGCGCATCTTAGTTTGATAGTTTCATTGTTTTTTTTTTTTAATT
TGATCCTCTTATATTAGCCTAATCTAGAAGTAGGTTTGATAGTTTATTCTAGTTGACTTTAAATTGGGTT
ATTGCAATATCAATTATAAATTTTAACATTGAATTAATACTAACTAACAAAATAAAATTGAACTTTTATT
GATTCAAAACCATTAAAAGTTTAAAATTTTAATTTTAAAATGAAACAAATATTCAATATTTGTTTTCAAG
AAATAACATCTTTTCCTCAGCTTTTCAACTGGATATTTAATTAAAACAAATAGATTTATTATCATTTGTT
AATATATAATTATTGGTTTATTTTATTAAACCCAAATATAAACTTTTTAACTTGCATTATAATTTTTCTC
AATAATAATAATAAAAACGTTTACAGCACCTATATTTTATTTACGTTGAGAAAAAAAAAATCCCACCCCA
CACTCAACACACACAAAAAAAATCTGCTGTGGCCCCGAGATACACCGCCCCCACTTTCATATTTATCCAA
TCGAGGCCGACACCACGGGTCTCACTCAAGGACAGCCATTCACGTTCTCGTTCCACGTGTCGCCAAGGCA
TTTGCAGAACATTACAGCGTACCCCTCTCAACTTTTCTACTTCCTTCCCTCCTTTCAGACTGCTACTTTA
CACATTCCTCCGCGTATTGAACACGCTTATGCCTCCACCACTCCCATGAGTTCCATCTCTAATTACTAGA
CATCTAGCCAATCATTCAAGCGAGTACATATAATTCTATACATGTCGGAAGTAATACGTTTTGATTTCCT
CCCTTACTTAAATTTACACGTCGGTTACAGAAGTGAAACCTGACTGGCCCAGCTCACTTGGCTTTATTAC
TTCTTAATCGCGTCAGCTCCGCGTTGGCTTCCAGCTATAAAAGCCCATCACCCTTGCTAACCCAAACCCA
AACCCAAACCATCAGTTACAAGCGCAAATCACTCGAAGCAGAAGCTTACTCATTTTAATTACTCTGTTGT
GATTTTGATCTTTTCTTCATTAGGTTCATATTGTGATTTTGATCAGT
Sequence ID: 110
Sequence Length: 3067
Sequence Type: DNA
Organism: Poplar sp.
CTAGTATAGATTTAACCCGTGAAAATTTCAATTTATAACATGAGTTCATGAAAATATTATAAATTTTAAA
TCAAAATCCAAATTTAAATTAATGAAATATTAAAGACCTTAAATGAAAAAAAATATATTAAATAAAAAAA
ATAACTCTAGCAAATCTAGAAAGGTGTCTTGCTTCAACCATTAAAGTAGGAAGAAAAATTCAAAACTTGG
GTAATTAGCTTGAATGGATCCATTAAGTCGAGTTAATTTAATAATATAATTAAAAAATAACAATAAATAA
TAAATAAAAAGACCCAAGATAACCCATATTATTTTCAAAATGAGTGAAATTATATAGAAAAAAAAAAAAC
AAGCCTAATCTTCAAATAAATCAAATGTTGACGGATAAAACCTAAAAACCATGAGCTTAAAAAAAAAATG
ACTTAATACCTTATACTACGTTGCGAGTTAAATATTTTTTTTTCTAAACTTAAAAAATGTTAAAATGAAT
GAAGATAGTGACATACTAGATATATTTTTAAAAATGCATATTGAGCAATTACTTTAATTTAATTCAAAAA
AATTCTTCTAGAACAATCTAAATATAATAAAGAAAAAAAAATAAAAAGACTTTAGATAACCCAAATTATT
TCTAAAAGCAATGAAAAACTTTGTACAAAGAAAAAAAAATTGTATAGCCCAATGTTCAATTAAATTAATT
GCTAAATGATGAAATTAGAAAACATGAGAATTTGTTTTTAAAAAAACAACAACAAATAAACCCGAATGAT
TCTCCTAAACCCAGGCTAATCAAATTACCAATCACCCCTAGGACCACCAATAATTACAAAAAAAACCAAT
ATGAAAATACCAACATGCCCCTAAAGCAAAGCCTGTGACTTTTTTAATTCTAAGAGCAATAAAGTAGTTA
CTTGAACGGTTGATGTGAAATGTTGTAAGAGCAGTATGGAAACCAAGGAAGAAAGGGAAAAAAAAAGCCA
ATCGCAACCAAACTTTTAAGAATTAAGTCACACGTGCCATATGGAGAGATAGCCCTTGCAATTGTGAATA
AGAAAACACTAAGGAAATTGATTTTTACTTGCCAAGAGCCACCACATACGCCTTTTGAAAATGACAAAGT
GACGCACTCGTTGGTGCTTTCCATGAACGCGCCAATAACTTTTTTTTTTATTTGTTTACCAATCAAATTA
CTAAATCATTCCTAGGACCATGTGATAATTACATAAAATAAATCAATATGAAAATGCCAACATGCCTCTA
AAGCAAAGCCTATGACTATTTAATAGCAACAAAGTAGTTACTTGAAGGATTGATGTGAAATGTTACAAGG
ACAGCATGGAAATCGAGGAAAAAAGGGAAAAAAATGACGAAGGAAACTGATTTTTACTTGCCGAGAGCCA
CCACATACGCCTTTTGAAAATGATGAAGTGACGCACTCGTTGGTGCTTTCCATGAACGCGCCAATAACTT
TTTTATTATTATTTGTTTACCAATCAAATTACTAAATCACTCCTAGGACCACGTGATAATTGCATAAAAA
AAAAATCAATATGAAAATGCCAACATGCCTCTAAAGCAAAGCCTATGACTATTTAATAGCAACAAAGTAG
TTACTTGAAGGATTGATGTGAAATGTTACAAGGGCAGCATGAAAATCGAGAAAAAAAGAGAAAAAAAAAA
TAGCGCCAAACCTAAAGCAAAGCATATGACTTTTTAATTTCAAGAGCAATAAAGTAGTTACTTGAAGGGT
TGATGTGAACTGTGGCAAGGGTGATATGGAAATCGAGGAAGAAAGGGAAAAATAAAATAAAAAAAAGTTA
GTAACTGCCAAACCTTCGAGAATTAAGTCACACGTGCTGCATGGAGAGGTAACCCTTGCCGTTGTGAATT
AGAAGACACTAAAAAAGCTGATGTGTACGTGTTGTGAGCCACCACACGCACCGTTTGAAAACGATAGAGT
GGCGTGCTCATTGGCGAATTCCATGAATGTGCCAACAACTTTTTATTATTATTATTATTTGTTTATTAAT
CAAATAATCTAATCACCCTTAAGACCACCCGATAATTACAAAAAACCAATATGAAAATACCAACATGCCC
CTCAAGCAAAGTCAATGACTTTTTAATTCCAAAAGCAATAGATTAGTTACACTATGCATGTCAAAATGAA
ATTCCAAAAAGCCCCTAAATCATAGTTATTTTTTTTTTGCTTTTAAAGATAATCAAGTAATTTAATTGTA
CAAAAATTTATAAAAATACTGAGATACCCTAAACAATTATAAAATAACAAACAAACCTAGCAAAAATATC
ATTTTACTCCTCAGGCATTCTTGAGTTTTTTTTTGAAAGGAAAAAACACTAATTACTCATACGAAATAGT
TTATTTTAATTTTTTTTTATGTGTTATACTTTTTTTTATTGCGGACAAAATTCGATTCTCTCTTTTTTGT
TTTTAACTATCCCTAAAAGCATTAAAATAATGTTATTTTATTAGAAGTGTGATTTTGATTGTTTTTCAAA
GTACTTTTCACTAAAGAATGCATCAAAATAATGTTTTTTTATTTTTTAAAAATTATTTTTGACATCGTCA
CATCAAAATGATCTGAAAATATATATAAAAAATTAATTTAAAACAAAAAAATAATAATTTTTTTAAAATT
TTTTTAAATTTTTTTAAAACATAAAAACAAATTTGTCCTAGTCTCAATCTAACCTTAGCCAAATTATAAT
CTAACCTTAGCCAAATTATCTATAGTAATGGTTTTTTTTTTGGGTAATGGTTCCTTATAGTAAAAACCAT
TTGAGTCAAACCTCCAAAATGACATAAATCACAACTAAGCCATACGTAAAAATAACCATCCTGGCTTGCA
TGAACACCACCACCTCTTAGGCTCTTGCCACCTCATACCACCTAAACTAAATCAAATACAAACCCCAAAA
CAGTAATTTCCCACTTCTCCTCCTCCCTTTATAAGCTCCCCACAAGACCGTCCTCTCTTTGCCAGCCACG
AAGCCACCACCACCCTCTGCGAAGTGCAAACCCATTCCTTGCCCCCCTCCCTCGAAC
Sequence ID: 111
Sequence Length: 3089
Sequence Type: DNA
Organism: Poplar sp.
ATATATGTGATGACTGAATCAGAAAGGGGTTTGAGTACTGATTTAGGATGTTCTACTGGAATGTAGGATA
AGCATACAAAGTGGGACCCGACTTCTCACGTGCAAGTATAACAGGAGTTCAAGGTAAGGGTTGCTCCCTT
ACTTCTTTAAATTGTTTAAATTCTTGTAATGCTAGTTATATGATAGACATTATGCGTATTTTTTACTACT
GTGTATCATGAGAAATATTGCTATGTGAACATTGCCTACGGGCAGTAAAAATATTGTTGCGTGAAAATTA
CCAACAAGTAGGAGAAATATTATTATTGTGGGAACATTACCAATGAGCAAGAAATCTTTTTACAATGTGA
ATGTTACCTACGGGCAGGGAAACAAATACCAATTCTATGTGAATGTTGCTTACAAGCAGGAGAAATAATA
TTATTATATGAATATGTCCACCTGGTGAATATGACTAAAGAAAGGAAAGATACGGTGAAATGAACATGAT
TTGAAATCTGAATTTTGGTCTAGACTTTGGGATTCAGAGTTTAGATAGGGGTCTATATTGTGTGTGTGTG
TGTAAATGACCGCCTATGAACTTGATTAGCCGACCTCGAATAGGGGCGGCTAATGATATTAACAAATATT
GTTAACATCGGCATGACCTTGAAGGAAATATAATGAATATTGAACTTGATTATCTATTTATATCATGAAT
AGTTAATGATACTGACAATTAATGTTTTTATCGGCAAACCCATAATAACTTGATTAACTTCCTAAGAAGG
AAAGTAAATGGTGTCAAGAGAACTTGGCATCGACATTTCCGGAAATATCTCCAAGTGATGGAGAGCAATT
GATTGACCATTCGGGTTGGAAATAGCTCAATGGTATCGATTGATTAAGTTGGTGCCGACATTGCTAAGGA
ATTGATTAGTCTATCACGGTAAGGGTGACTAATGACACTAATAGGAGTTATTAGTGTCGACAGCGACTGC
CTCAGAATTTGGGGCGGGTAAGAAAAATGATAAATTAGACAAAAGTTAATGTGTGATAATTGAAAAGAAA
ACTATAAGTAGGTTGTGGAAAAATAGGCGTTAGATGTAGTAAATGATCTTCTTTTGCGAATCAACTATGA
TTATGCCTTATCGAGTGAGTACATATATGCGTAGTTACACGTAGTGGTGACCCTCCATTATCTGGTTGTA
TTTTATTTATTTTATATAATTATTCTTTTATTTTGATGTTGCAGGTCTTTCTCATTTCCATTAGGAGTAG
TGTCTTAGGTGTCTAGTTCTTTTGTATGAATTAAAGAATTAACATTTCCAATCCTTTTGTATGACTTAGG
GTTAGCATCCTTATTTCTTCTGTGTGAACCTGAGATGAAATGGATGTTGTTGTATTTTCTTTACATAATT
ATATTATGGGAACGATGTCAGTGTTCCTTTATCTTAATCATAAAGACCTTTAGAATTTTATGCTTGTAAT
ATTAATGTTATTAATCAAATTTATTACTATTAGTTTGTGTTGTAAATTATATGTTCGAAATGAATCCCTG
GATTTGCAAAATATTATGATTAATAATAATTGTGGTCGCAATGCAATATGGATATATGTTGATTATGGAA
TGAACATGAGATATGTATAGGATGAGTTGTTGGTAACTTGGGACCTCCTAGTATAGGGAGACTCTACCGA
AATTTCAGTAAAAATTTTGTTACAGTGTAAAATAAATAAATAAAAAAACTCATATTTTTACGTACCAAAT
TGTACGAAATTAATTTTAAATGCTTACGAAATCGTCGGGTGTTACATTCAATTTCATTCTTCAATCCCAA
TTTGTGATATATTTTTTTTAATTTGATACTTGTTATTTTGATTTCTAATTTCTTTTCTTGACTATTTTGT
ACAATTTTTATTTTTTTTAATTTAATCTTCCAATTCAAAATTATGATATATTATTATTTTTCAATTTGAT
CCTTGTTTTTTTGAATTTTTTGTCTTTTTGTTTACAATTTCTAAGCTGATTTTTTCTTATTTTCTATTTT
TTTGGGTTTTGTTTCCTTATTTTTTATGATTTTATTCTATTGTGTTAGCTCTTTATATATATTTTTTTCA
ATTTCACTCCTACTTTTTTTTAATCTATAAATAATTTATCAAATTACAAATATTTTTCAATATCATTTCT
TATGATTTTTTTAATCTTTCAAGTTTAGTACCCATTCTTTTTAATTGCTATTTATTTTGTTTGAGATTGT
TTTTATTTTTTTTTCAAGTTTCATCATCCTTATATTTTTTTCTCTCAGACTTTATCCTCATTCTTTTTAT
CGTTGTTTTTTTTTGTTGCCTTTACAAGTTTTTATATTATTTTTTTTCAACAATTTCATCCTTTAAAATT
AAATCGATTAAGAGCTAAGCTTTGAGTTCATGATTTAACAAGTGGCAAGTTTTAGAGATTAGATCAGGTT
TAGGAGGTTCGTCTGGATTTGCTTGTTTTTTTTTTAAGCTTATTCTTTTTTTATTTTTTGAGTTTTGCTT
TGATTTTTTTTCTATAGTTTATCTTTTATTAGGTTAGTATTGTGTTATTTTATAATCTCACGTATGGTTT
TTTTTTTTTGTTAATTTATTTATTTTTGCTATATTTTTATTTTTATATTATTTAAAATATCGATTGGATG
AATAAATTGAAACTTTAAATGTTTTTACCCTAACATTTTTTTTTTATATTAAAAAAAATGGCTCAGCCCG
CGGGCAACACGCGGCCGACAAATCTAGTTATTATAGTCAAACCTCCAAAACTCATAAATCACTTGACCTT
CGCAAAAGACATAAAGAACACCACTCTTTGCTAAAAAAGCCTTCCTCTATGTGGCAGCATGAACACCACC
TCTCCTCTCTATCTAGATTCTAGCCACCTCATACCACCTAAATAAATCCATAACCCCAAAACTGTAATTT
TCTATAACCTCACCCACTCCTCTTCCCTTTTTAAACCCAACAAGATCCTCCTCTCCCCTTGCCACTCCCC
AGCCCACCTTCTTCTTCTGCCCCCCCGAACGCCAGTTCCTCTCTCTCTCTCTCTCTCTCTCTCTCTCTAT
ATCCCTCCA
Sequence ID: 112
Sequence Length: 3204
Sequence Type: DNA
Organism: Poplar sp.
GAAAGAACATCAAATGAGGTCATAATTAACTCTAAATTAACCCTAAACAAAACCACTAACAAATTACGAC
CATGTTTGGTGGTATCTCAAGGTGCTGTTAGAATCATCTCGTTGAGATCTTTTTAATGGTGCCGAATGTG
TCGAAAACAAAGCTTTGGTGTGTCCCAGTGACCCATTTTGGATATTCATAAAATTTTGAGTTGATTTTTA
TGCATGTTAATTATATAGCGAGGGACATGGTTTTTGGAAAAACTGTTATTCCCAATAATAGTTGTGTTCA
ATCGCTATTCTCAACAGCAGTTATGTTCAAACTATGTTATTTTTCTAATAATTTTCCAACATGTGTTACT
TTTCCAAATATCTTATGAAATTATGTTAGAATTCTAGAATACCCCTTGGCTCTCCTGTTTTCCTTTTGAT
AATCAGAAAATACTAGTTATTTGACTCGCACTCCATTGCGAATCGGATATTTGTTTTCTACAAAAAATTA
AACATGTTGGCTTTTTCAAAATGTTTTTTTTATATATATAAAATATAAAGTATACATAAAAAAGTTAATG
CATGCATCTAATTAAATAAATTGAGAACTATTTGTGTTAATAAATGAAAAAAAAGTCATTAACGATAACT
AAATACATATCTAGAAAAGTCAAGAGGCGAATGTAGATCAAGATATTTAATCTCATAACATGATATATTT
ACCCAATTATATTCAATTACTTTATTTATTAAATTAATTTTTTTTTATCTAATCAAATGATAGTAGAAAT
AGACACACACATATAATTGATTAAATAAAATCAAAGAAAAAAATATCTCACAAGGTTGCACTCATTAGAA
ATACCATCCAAAAATAAATATTTTTTATCTTTAAAAATAAATTTCAATTATATAAAATATAAAAATTCAT
AGATGAATTAAAATAATTGCTTCAAAAATTAAATACAAACAAAATAATAATAAAAACAACCACTATTTAA
AAAAAACTAAATAAAAAAAATAAAAAATTAGAGAGAAGGGGGTATTAATTTAAATATAAATTTTAAAAAA
ACTATTTTAAAAAAATTATAAAGGCATGTTTTTCTTAAAAAAGAAAGGCAAAAAAAAAAAAAAGAAAAAG
AAAAAGAAGAAAAAGGCCAAGCGCTACAAGGCTTGGCTCATCTAGAAAGGGCCTATGTGTTGACCCTTTT
TTTAAAAAAAAAATTGTCGTTTGCCCTATTTTTTTTTTAAAAAAAGAGAGGCAAACAACACATCATTTGA
TATTTCTCAAATTTCAGCCACTATGGTGCCCGAAAGAAACAAGGTCTATATTTTTTAGACATAAAAATTT
ATTTTCAACCTATTTTTGACTTAAAACACATAGAAAAGAATTTAAATACCTTTATAGCCCCATAAATTGC
CATAAAAAAAAACTAAAAAGACCACGCAAAAACTCGAAATCAACTTGAAATCAAAATCTTTTTATGTCTT
TTTTCATTTTTTTTTTATGATTCTTTTTCTTCTCTTTAAAGCTCAATGACTAAAAGGTGAACCAAACTAA
AAAGGATAAAAAAGAATATTGAAAAAATTATAAGGCAAAAGTCAAAACTAGCAAAAAGGCACCAATGCCA
ATTTTTATTTTAAAAAAAAGCTCCCACTTTTTCTTTATTTCAATTTTGGCTCTTTTTTTTTTTTTATTAA
CAATAAATTGGATTTAGTTTAAGATAAAAATGACTTTATACAAAGTAGATTGAAACCCATAAATTCCAAT
CAACACAACATCGAAAGGTAAAATTAAAAAAAAAAATTAATGACCTATAACAAAAAAAAAAAAGGATCAG
AGTTGACACAATAGTTTTAAAAAAAACAAAATGGCCGCTATTTTTTTTATTTCAATTTTGATCCTTCCAT
TTTTAATTTTTTTGATATAATAAAATCCAATTTGTTTTTTGTAAAATCGAGCATTAACATGACGTTGGAG
TTTTTTTGAGATCCTAATAACCATAAAAAAAACAAATCAAAACAAACCATCAACACCATATCAAAGCATG
GAATTGAAAATAAAGATTTTGATGATTGGAAAAAAAAATCAAAATGAAAAAGAAAAAGAAAAAAAATATT
TCAATCTATAGTATTTTGTATATGAGAAAACTTTTGTTTTCCCACATCTTTTATGTTATTGTTAATTTTA
ATATTAAATTTAAACTCTTTCAACTCTTCTTTTTTCTATATCTTTTTGAAGATCTCCAATAAGGCTTGAT
ATCTGTAAACTGTAAAGAACAAAATATGTCTATCAATGAAAAAAAATTAATTTTGCAAGTTCTTCATTAA
GTGTATAGTTCATCAGTCAGTAATAAGAATAAAAATTCATAATAATAACTAAAAATAGAAATATTTAATT
TGTGATTAAATTAGAAACAAAATGTTGTGATTGTTTTTTTACCTCATGAAAGTCTTTTTTACCTTTGTAA
ATGTTGTGATTGATTAAATTAGAAATAGTTAATTGTTTTTTTACCTTCGTAAATGAAAGTTTTAAAAGGC
ATAATACTTATAAGTAAATCAATAAAATTGAAATTTTTCAAGAAGCAACCAATCACAACATGAGTCTATG
TTTGTGCTAGAGTTTTAATAAATTTTGAATTTTTTTGTTTTAATTTTTTTATATTTTTTAATTATTTTAA
TGTCCTGAAATCAAAAATAAATTTTTTAAAAATAAAATAAATATAATTTTAATACATTTTAAAATAAAAA
ATAATTTAAAAAATAAATATAAGCAACATTATGCTCAAAATGCAATCATAATTAAATCTCTTAAAATATG
ACTCATTTTTCAAGTATTCAAGTTCAAAATAAGTTATTTTAGATTTTTTTTTATTATCCTTAAAATTTAA
ATAAGAGACAGTAACACGGGAATAACAACAAATGCGGGACTCCACAGTAAGGACACTTATCTTCTTCTTC
TTCTTCTTTTAAAAAAAAAAAACAAAATTAGAAAAGAAAATAAATGCTTGAGCTTAATCTAAACCCAAGG
AAAAATCCAACGGCCAAGATTGGGTTTAAGAAGAAGTGGGTGTCTCAGACTCAATGTCTTTCTCTATCTA
TATCAAGGAAGGCATAAAATAGAAGTGTTGGTGTCCTCTTGTGTCTTCAGAGTCATCGTCCTCTTTCACC
TGTTCTTCTTTTTTCTGCTTGAGAGTGAATCGATCCAAGCCTAACTAAATCATC
Sequence ID: 113
Sequence Length: 3185
Sequence Type: DNA
Organism: Poplar sp.
CCTGCAAAATGTGAAGGATAAAGTGCATAATCATTTCATTTACGATTGCTGGTTGTGGTCATTTACCTTA
TCCCTTCATTTATTTGTATTTCAGATTGTTCAAAACGGCCAAACCATTTTCACACTGTGGCGTCCATATT
TTTATGTCCTGACATAGTGCACCGCATTCTTGAGCAAGTGTAGCGGATTTATCTCCTTGGCTTCTTTACT
GAAGGTTTGGTAATTCTCCTTGCAGAGAAATCCAAAGCTAATCATGGTGTAATGCTTGTAACATTAAAAT
CTAGAGCTGATGATTTGTGACTGAGAGAGAAGAGTGATTTGTGACTGTAAAATGATTGAGTTTAGAATTT
AGACACAGCGGGTGCTTTTTTATTAAACTATGTAAAAATTCGTTGTATCTGTAACAGATACCAGTCGATA
AAATGGTTCTTTTGTCCAGCTTTAATTCGATACCTTTTGGTCATTCAGCTGAATTGTGGGTGCTAACTTG
TAAGACCTTTTTCGACTCTTCGCTTTTTCTCTCCCTGTTTTCGAATGGACTCAGATACTGGGAAAGAATG
ATTTGTTTTATGTGAGGCGAAGAGTGAAACGAAATGGCTGTTAGTTGTGTTCGGAAAGGTTTGAAGTTCC
AATTTTAGTACTCAGCAGTGCTTACGGTTGTGATTTAGAGTGTTAACATTTTTTTAAAATATATTAAAAT
AATATATTTTTTATTTTTTTAAATACTTTTTTATATTAATACATCAAAACAATATGAAAATATAAAAAAA
ATAAATTTTTATAAAATATTATTTGAAACGTAAATTTCAACCATAAACTAAGTCTTATGGGAAAATTATT
GTAGCTCAAGAGAAAAATAACTATTTATTACAATAGTAAGTTGAAATAGTGAAGTGACAAGTTTGGTCTT
CTCACCATGAAACACAAAGCTTTTTATTAGAGATGAGTTATTTATTTAATTGGGGGTAGCTTGGTATTTT
CTTATCTTATGGTGCATTGAAATTACTTATATGCTTTAAAAGCAAATTTTTTTTAACTTGGGTCTAGATG
CTCGTTGGACTTTTTATTTTTATAAGCATATTGTATAAGTATATTGTAATGACTATGTAGTTTTTAGAAT
TTTGTTTTTCAAAGAAATCTACTTCATCTTGGTTTTTTTCATAATTTTCATGTAGTTTTATGAGCAAATT
AGTATTTTTATCATTTTTCTACTCTCTTTGCCTCGATTAGTGTAATGCTCCTAAAAAATAGCAATAATCC
TTCAATTTATTTTTTTTCTTGAGATTTTAGTCTTTATTTTAATTGCAATTTTTTTATTTTAATTAATTAT
TTCAAATTAAAAATTATTTTTAATTTCATTCTCTTTTAATTTTTTTAATTTCTTATATTTAATTCTCTCT
TTTGATTGCTAATTTTTATAGGTGATTTTTCAAGTTGATATTTTTATTTTTTTTTGCAATTTCATTCACC
ATCTTTTTTTTTCCTATGAAGTTTGATCCACATTTTTTTATTGTTGTTTTTGCAAATTTTTTGAACATAT
TTTTTTTATTTCATTCTTCAATATTAAATTTATTGAGAATTGAACCTCTTTATTGAACTCGGTATATGAT
TTCACACATTGTAATTTTAGAAGATTAATCCAGGTGTAAAAGATTCATCCGAGTTTGCATAGTTTCTCGT
ACCACTAAATTAGAGTTGTCTTTTTTTATATTATTAAAATAATTGAGCTTGTCAAATCAGTTGACTTGAT
GATTTGAGTTTATAATTTTTTTCTCGATTTAATCCTTCAACGTTAAATTAGTTACGAGTTGAGATTTTTT
GTTAAACTCAAGTCTAAGATTTCACGGGTTTTGAATTTAGAAAATTAAACCATGATTGGAAGATTTACTT
GAGTTTGCGTGGTTTTTCATGTTATTAAATCAGGATTGTCTTTTTTTACATGTTATTGAATTAAATGATC
TTATGAAACACAAGCGACTCAATGGTTTGATTTTTGGATTTTCTTGACTTGTTTGGAAACACTTACATTA
CCTTGACTTTCTTTTTTTTATATTCGGTTAAAATTCTTCTAGCCTATGAGGTAGCGCCAACCACCAATCT
AGGTTTATTTGCGTCATTTTTTTTTGTTTTTTTAGATTTAATTTATTCTTCTCATTTTTTACTTTCAATA
TTTAGTTCATCATAATTGACCTTTATGATTTGTGTATAGGTTTTATAGGGTTATCACATTCTCCTGCCTT
GAATTACAGGTTTTGCAAGTTAACTATGGTTGGTCGAAGTCAATTTAATGCGTTATCATCTTAATATATA
TATTTTAAAAAACAATATGTCAATATTTAAGTACTTTAAAAGGTTTTTTTTTTATGTAGCATGTATCAAA
TTTTTAAGTTCATGATATTTTTATATCTTTTTAAAAGATTTTAAAAAAATTATCAGAGATCTATATTTTG
CGGGTAAAAAAAACTAGCTAAGTTTTAAAGTTCAACCTCTTAAAACACTATTAATAAGATAAAAATATAT
AAATTAACAAGGTTTATAAATTTGTCAATTAAAATGTTATTATTTTTTATATTAAAAAAAAAAAGCGTAC
ATGCATTGCCGTGTGATAATTTAATCATACCGGGTTGGTAATCTCGCTTTAGGATAAAAAGACTTGAAAA
TTATATTTTGATGAATTTAGCAAACTTAAAAATTTTGTATAATGCGGACTAGTGTCCGTACAAAAACAAA
TTACCACTTTTTAAATCAAATCCCATAATAATAATAATATAAAAAAAAATGTATTGAAGTAAAAAATTAT
AAGAAATTTGAATTTTAAAAACTTTTCGATAATCAAACAAATCTCAGTAACAATAACAAATGCAAACACC
TCAACCCATCCCAAACAGTACTACCCCAAGCACACCGTACTAACTGTCAATCAGTTAAATCCAATGGTTC
AAAATATCCCATTTTTAACTGTTAATCTCTTAAATCAACGGTACAAACTTGCCCGAAATCACTGCTATCA
TTGAAAAAAAAAATCACAACTTTAGAAACCCTAAAAACCTTCTACTCATCTAAAAATGCATCCCCTATAA
ATAGCACAAATTGATAAATCAGTTGCTCTTCACACTATCTCCCTATCCTTTCTCTATCGGTATTTTGTTT
GTTTTGTGATTGCAACTCTCTTATCAATCGCGGCC
Sequence ID: 114
Sequence Length: 3115
Sequence Type: DNA
Organism: Poplar sp.
ATCTCATACATGATTGGACTCAACTTATATGTGATGAATCCTCCTTTGCAGGAATCAAGTAAGAGCATCC
AGACAGAAGCCTTTCATGTTTTCAAGGTACAATTTAAAGAAAGAAATTGTGAATGTTTCCTCTTTGATTC
TTTAACTTCATGTGCTTTTTTCTTTCCTCTCTTATTGATTTCCCCCCCCTGTACTGCAGCTTTTTGTTGC
AAACCAAAATAAACCTCCCGACATAGTTAGTATACTTGTTGCAAATAGAAGCAAGCTTCTTCGTTTGTTA
GCTGATTTCAAGATAGATAAAGGTATAAAAACGTAATACATGTCTAGTGAAATTATGCTGGCTTCAGCAC
TGTATTGACATTTTTTTTTTTTTTTTTGCAGAGGATGAACAATTTGAGGCAGACAAAGCTCAAATTGTGA
AAGAAATAGCTGCCCTTGAACCTAGAGAGTAGCCATGAGTGTGCCTGGTTTCTTGTAATCATTTTACATA
ATTTACAGCTCTCATACCCTGATGACAATATCAGTATCCAACGAATATATGTTAAAACTGTATATTCTTG
TGCGAAGGGAAGAGATAAATAAAAAGACATGAATTGAGTTCAATCCAATACTTGGCTTTGAAGATTGTTT
TAATGGGTGGCAAAAGAACCCTGTAAGAGAAGGAGTTCATGTTTTATCAACCTCTTAGCTTCACCATCTG
TTCATCCCTCCCCCTTCTCTTGTAGACAAAAGTCACAAGGACAGCACAAAAAGAATTGTGGAAATGAACT
TTAAACTTGCAATTTATTTATTTCAAAATAGAACTAATGAAGTCTGAGGTTTACAAGACCACCTATATGT
ATGCCAGAAAACAACAGTTTGAAATTTGAAAGCACAAAGGAAATTGAAACAAATAGTAACATTTGGGCTT
GATTTCTAATTTTGGGATATTCAACGGTTCAACATGCAATATGGTGGCTTTCTATTAGAACATTACTGGT
AGAATTACTTGAATTTTTTTTTGTTTTAAGCTGCTAATTTAGTTTGGCACGATTATACCTTTTCTTGTAC
CTGGATTTATTTTATTGGTCAATAAAAATATTTGTTATACTATTAATATAGTTTATTTTAGTCAGATTTC
TATCAAGTTGTGTTAGATTCTCATTTCAGAACCTTTAATTTTTAGGTGGACTGCATCATCTTTGTAACAA
CTTCTGGTTCCTACTGTATTTTTATGCATTTAGAAGATCTCACCACTTCTATGCTAACCATTAACCAAAC
TTAACTTAGGGTATCTTTGCTAATGGAGATAGAAGTATTTTCACTGTGGGAGTTAATAGCTATAGATGTC
TGTGGTCAGACTCAAACCTAATCTCATGGAAGGAGAGTAAGCAGCTAGCAAATGTTCATGTGCTAAAGGA
TGCTAAAAAAGCACATCGCTATGCTATTTCCACCCTCAAGATTATCATGGTGGCAGTGGTGGTCAAGGAC
AGAGCTAGAAATTTTGGGGAATTAGAGAAAAGTTTAGGAGGACCAAAATTTAAATTTTTTTATTTTTAAG
ACTAAAAATCTAATTTATTGAAGAAGGGAGGCTAGGGAAAAAATATTTTCTTGCGAAGGCCTCTGTCCTT
GCAGCCCCCTAGTGGTGGTGACTGGAGATGGTACAAGGTACATGAACCTTCCACAATTATGGGTTTTCAT
AATTATGATTGACATCGGAGTCACCGGTGAACGTCACCAAACACTGCAAGCACAAGCATATTCCAAGCAT
CTACTTCCATTCAAAAGCAGAATGTTCTCTGCTTTGACTGTTAGTCTGTTACTAGCACTCTTCGTTGTCA
TAAAAAGGAGATGCAAAAAAGAGTATAAGACAATGGGGTGTTTTCATTTTGGAAAATCTACGACTACACC
CTCCAATTATTGTGTAGGTTTCAATTTGGTCCTTCTCCTTCTAATCTCATCAATATTGAAATTGTGCAAT
CCCTCTTCCTCATTAATTGGATGTTAATTTTCCCATAAATTGCCTGCAAAAATCAAGTTTTCCAATTCAT
ATCAAAATTCCTCATTCTTAACCTTCCAATTTAATTGCCTTAATTTATGCTTAGCAAGTGACACCAAATT
AACTATCATTCATGTTTATTATGTGACTTCGAAAATTTCTATCATTTAACATACAACTATTTTTTTAATA
ATGAAATTAAATAATAAATGTTTGTGAATATAATTAGAAGGTTAAAATGGATTAAATTAGAAGTAAATTA
ATACACAAATTAGCATCTAATTTAACAAAACAAATCACCATCCAATTAACAAAAAAAAAAAAACAATTTG
GATAAGTTAGGGTATCATCGCTACAATCACAAAAAGAAGAACCAGACCGATACCTATCCAAGAAATGAAG
AGTATATTAATAGTTTGCCCATAAATAAAAGGGTTGAAAAGACTCAAGAGAAATCATTGAAGGACTTAGT
TTATTTTTCACCTGGACTTTGCTGCAAGAAGCATTGTTTTAAAATGGGATTGGCCCGGGTTGATTCAAGA
CTGTACTGACTCAGGTTTAGAGCTGCTAAAGTTGAAAAAAAAACTTATTTGATCTAGTTAAAAACTCAAT
TAATCTAATAACCCGGTTAATCTAGGCCCGACTCATTCAAAAATCAGTAGGTTGATCGAGACCTTACTAA
CTCAAGTCTAGACCTGTTCAAGTTGAAAAAAAATAGAAAAAGATAAAAACTAGTTTTGTTGACCCTTGTA
ATTCAATAAACTCGAGTCAAACCAATTCAAAACTCAATCATCAACCCGTTAACCTTTTCTTTTTCAAAAA
AACCAGGTTTTTTTTTTTAATTCATAACCCAAGTGTTATCCCCCGATTAACAGGTTTTAAAACTATGGCA
ACAAGGACGCTACTACACTGGAAATCCAATCAAAGCAGAAAAAGAAAGGCAGATTCCTCACCATAAAACG
TGCTATATAAAAAGAGGAAAAAAAAAAACAGAAACCAAACGGTCCTTTCTGTGGCTGGCTGCTGCTTCCT
TATGTACGTAAAAAGACACATAGCCTTTCATCATTTTCATCATTATCACCACAATAATTGACCTCCCTCT
GCCATTCTCTTCTTTGTGCATTCAAATTTATAAAC
Sequence ID: 115
Sequence Length: 1726
Sequence Type: DNA
Organism: Poplar sp.
CCCAATCCCCAATAAATGAAATGCCGGAGGATAAAATCAAGGAAAAAAAGTGATCACATAAAAGGACTAAAAAA
ATAGTAATAAAAAGAATGAGAATAAAAATAAAAAAAAAATAAGGCAGAGGGCAGCTACAATTTTTTAATCAGAT
GGTGAAATGAAAAAGAAAATTAACTTTAATGAAAGGGGAAAAAAATCAATAACAAGTAACAAATTGAAAAAGTA
ATACACCATAAATTTGGATTGAAAGATGAAATTAAAAACTAATAAAACTTTTACAAAAGAGTTACAGAAAAAAA
TAAAAAATCAAAAGAATAAAAATCAAATTGAAAAAAAATAACATATGAAAAAATAGGATCGAAGGATAAAATTA
AAAATAAATAAAACTTTTATAAAAGGATCAAACATAAAAATCAAAAATAAAAAAAAGAAGGATTGAAGTTGAAA
TATCAAGAACAAAGAAAACCAACCTGTAATTTTAAGAGAAGAAAAGAGAGAAAAAAAAAAAACCCCATTGATAA
TAAATCATCTAATCACCATTGACATGTGTAGTATTAACAAAAAAAAAAAAAAAAAAAAACAACAACAAAAACTC
ATCCATCAACATGTAAAAAAAAGACATTTTTCCACTGGGAGGAGTCGCAAAAAAAAGTGAATGATAAAATATGT
CAATAACCATAATGAATTTATCTTTTGTTTTAGTTGTTTTGTTATTTTGGAATTATGCCGGAAGGCTCGTGGAA
GCAAAACTAATATTTTACTATATACGATATAAAAATCTGATACTAAATAAAAAGTAAGGTGCACGATAATCAAT
ATAATCATTTGAATAAAAAATATATTAGGTCAAAAAAGAAAGAAAAAAAGCTGATATCACGCTAACTATTAATT
AATCTGGAGATCTAAAATTATCTCTTATGAAGGATTCCTTGTAATTTTAATTATTTTCTCTTTTATTTTTAAGT
TTAAATTACCTCAATTAATTTTGGACTTTGGATCAAATTGAACTTAACCTTTGATATTTGAGTATCATTAAGTA
ATTAAAGTTAAAAATAATTTCTATTAATGTAGCCATTTTTAATTACTGAAAATCACCCCTCTATTCTCGTTATT
TCTTATTTTATACTAGCCATTCTCAATTTCACCCCTCCTTTGCCCTTACTTTTTAGTTTTTACATGTCTTTTAT
GTCATCCTTTTCTCTTAAATGATAATATTCAACAAAAAAGTATTAGGATTTTAAAAATTAATTCACTTGCCAAC
AGCAACGTGGCGCTTCTAGGGAACATGATGAGGGTTGGTTCTGAACAAGTCTAAACTACAGGGGTCAAAAGGTA
ACATGTCTAAATATATGAGATGGAAATTTAGCTTACCATTATAAGAATCACATAATGGGCCTCGCCTTCGTAAG
GTGGGCTCTATAACGCTGAACCCCTTAAACCACTAGAGCCCACGTTTCACTGCCTTTAAAACCCCCCTCTCCCT
CTCGTTTCCAAACCCTAGAGATTATTTTGTGTTGCAGCAGCGCAGCCAGAGATTAGAAGCAACCAGCCTCAGAT
CTTATTTCCACAAGGTAAACAAACTTTTCTTCCGGAACACTGTTTGTTTATAGATAGCTGGATGCAATCGTTCT
ATTTTTTTTTTCTTTTTGCGTTTAAATCGTTGATTGCTGAAACTTTGTCTACTGATTTCTTATTTTTTCTTTGA
TTTTTTTTTAATGGTTAAAGAAAG
Sequence ID: 116
Sequence Length: 3035
Sequence Type: DNA
Organism: Poplar sp.
TAGTCGTGACAAGAAAAGACCACTGGCTCAAAGGACACAGACTTCTATGAGTGCTCTTAAAGAAACAGAA
CATACTCTCATGTTGATGTGTGTGCACTAGCTAAACATTGGAGGTGATGGGTTAAGGTCATGAAAGAAAT
GGATTCAGAGCTGCTGGAGAAAAGTATTCATCTATTAGCCTTTATTAGTCCAGGAACTCATCGCGTTGGA
GAATCTTCCAGAGGAGTTAGAATTCTGCAAGAAACCATCATTTTTAAAAAGCAGGAATGGATTGTTTTCT
CTTCCACCTCTCTGTTGTGCTCAAAGCCCATGTCTTCTGCTTTCTCAGTCATCACTACTGCTACTGTAGG
CAAGGGCCATTCAACTAAATTACCAATCCAGTTTCTTCAGACTTGGTTGCCCTACATATTTATAGGATCT
CTTTTCTTCTCTTGAAATACCTCTGTATGGAAACAGAGGGCGCTGCTAAAAGATCAGAGGAAGTGGGGTT
TGCTGATCTAACTCAGATTCCTGAGCTGCCAATGCCGGAGATATTGCATGGCTTGCAGGTAAAAAGTGCT
AAGTGCTCCTGTATTATCATCGACTTCAGTCCTCTCATTGAAGGGATGTAAACGGTTTGAGATCATGAAA
TTGAGGGATTACTTTTGTAATAGTGACACTTCTCTGAGTAAAAGTTTTCACCATTGGCAGTAAATGAAGT
TTCACCATCTATGAGAGATTTCTGTGTTGCTTTTGATGTGAAAATACTCTATGCTTTTGATGCCTGTACT
GACTTGTTATATTTTAAAATGCAGGATCAGGCAGTTGCTGTTGTTCCGGAACTTTGTGGTTCTAACAAAT
CAAGGTACATCAATCCTGAAATCCAGTCAGTCTGTCTCTTACTACTACAGATAATGGAGATGGCCCTATA
TTTGGAGTTCTGCGTTTTACAGATTTGTGGAAGAAGCCCTGTATTAGGGCGTGTGGAGGAATTTTCGAAG
GAAGCAAAATAGTTGTCAAAAGGTAACAGCCCTTTCATACTGCTAACTATATTCTGAATTAATATTTTAG
ATTTACTATTATAATTGCCTTTGCTTTTTTTTTTTTGTAAGCCATGGAAGTCCATGCATTCTTTAAAGCA
TCAGTCACATCCTTGAAACATTTAACATCTCTTGTCTATCCCGTATTGTTACAGACCGAAGGTTTTAGGC
GAACTTAGTTGGGTGAGTAACAGATCATGAAAATCCTGTACAGATATCAGAATTAAGCATGGCCAAGATA
TTTGCATGATTACAACGTAAAACTCTGTATAACATCAACATGTGACAATATCAGAACCATACATCATTGA
TTATCGCTTGGATTTTCATGGTTCCTCTAGCTCAGTGAAGGAATATCACACAAATATAATGAAGATGGTG
CCAAATATCATCGATCTGGCGTTGTTTCGCAGTCATCCTCTTTCCATGATTTTAAGGTGCCCTGTTACAA
GATATGCCTTCCCAAGGGATTTTAATTATTTCTCGATCATGCACCGATGCTGCCTTCCAGCAAGGGATTA
TAATTATTTCTCGATCATGCACCGACTGCTGTAATGATGTAGGGAAATGAATTTTTCTAGGGGGACTGAA
TTTTCACTTTTGTGCTTGAATGGGCCCTGCCCCCTTTGCATGCGTGATGCCACAAGGTAGCATGTTTTTG
TGGTGAGACAATTGTTGAAAAAAAAACAACTGCATATTATTGCAGCTCTTGACAAGACATGTATCTTTGT
CTCTCTTTTTATGTAGGAGGTAATGCTATTCATCCAGGAACAGGTCTCAAGTAATTAGTTACTCCAATAG
TATCTATCAATCATGCGTTGTCATGTAGAATTTCCCTTCATTGATTCCAGATCGATATCCATGTCACCAA
AAATCATCTTGAAAAATTAATCTGGTATTTATGATTTTAGTGGCAGGGAAGTTTTTTTGAATTTTTTTAG
ATTTTTTTTATGATTTTATATAATTTTTGATGTGTTGATGTCAAAAATAAATTAAAAATATATAAAATAT
TATTTTAATATATTTTTTTAATTTTTTTTGAAATTGCACTAAATGCTGTCAGTTTTTCTTTTTATAGTTA
CAAACATTCTATTTCCAGATGACTGTACTAAAAAAAACATACCGTTATTATTGATATATTAAAATAATAT
TTTTTTATTTTTTATAATAAAGTTGAGGAATTTCAAAGAGATTTTGCTGGCGTGTCAAGCATATTATCAA
GTATTTTTTTTTTTTTTAATTATCATCCAACTACGGTAGTGGGCTTAGTAAATGAACGGGTTCCTTAACG
GTAGTGGGCTCCCCTACCACGTCACAGCTGTCAGTTACCACGTTTATTCAATAATTTATTAATGTTTTTT
TTTATTTGCTTAGCATCGAATAGTTGTTTGGAACTGCCGTTTAAATATATTTTTTAAAAAGTTTTTATTT
TTTTATTTTTAAATTGGTTTGATGTTTTAATATTAAAAATAAATTTTAAAAAATAAAAAAAATATTTTAA
TATATTTTTTTAAAAGAAAATAAGCTTCCGATGCACCGGAGAAAACTTGGTCACCGACGATTAGATCATT
AATGCATAAATATGATATTCATCGTTGACATTCACTAGAAAACAAAATCAATAAGGCCACCTGATTACTG
GTGTTTTTCAAGATAAAATCCAATCAAATCACTGACAAACATTAATCATGGCGCATGAAATTTGTACGAT
TTTACCTATCCCTCATAAATCTCGCCGTATCCGAACAAAACCACTTCCAAACGACGCCGTCTCTCCACGG
TGATAATAAAGCCAAAAGGCATATACCACTTAGGATCCCTTACTTAACCCTAGTCAAAGCAAGCCTGACC
ACAGTTAATCCCAAACCACCGCGATCCTTCCACGTGTCCTGATCTAGTGATCCAAATCCACCCTCTCGCC
CTATAAAGTCACCCCCAAAACACCCGGAAACCCTATCAAAACAACTCTCGTTTAATCCTTCTCGGAAATC
AAATCCCTAATCTCTCTCCTTTACA
Sequence ID: 117
Sequence Length: 3062
Sequence Type: DNA
Organism: Poplar sp.
CAGTTCTTCTTACACCGATATTCTGGACAACGTGATACCTTATGTTTTTTCAGAGAAAGCTGATCTGATT
TCTTATTATCTCAGTGCACCTGACTTTCCATCTGATAGTCATGACAAGAAAAGACCACGGGCGAAAAAGA
CAGAGACTTCTCTTAGTGCTCTAAAAGAAACAGAGCATACTCTCATGTTGATGTGTGCGCTAGCTAGACA
TTGGAGGTCATGGGTTAAGGTCATGAAAGAAATGGATTCAGAGCTGCGGGAGAAAAGTATTCATTTATTA
GCCTTTATTAGCCGAGGAACTCATCGCTTTGGAGAATCTTCCAGCAGAACTGCCCCCCTCTTATGCGCTC
CTATCCTTAAAGAGGAGTTAGAGTGCTGCAAGAAACCATCATTTCTAAACAGCAGGAATGGATGGTTTGC
TCTTTCACCTCTCTGTTGTGTGTCAAAGCCCAAGTCTTCTGCTTTCTCAGCCAACAGTAGTGCATTTGTA
GTCAAGGGACAGTCAACTGAAATTACCAATCCAGTTTCTCCAACATATTTTTCAGACTTGGTTGCCCTGG
AGATCTACAGGATTGCTTTTCTTCTCTTGAAATATCTCTCTATGGAAGCAGAAGGTGCTGCTAAAAGGTC
CGAGGAAATGGGATTTGTTGATCTTGCTAAGATTCCTGAGCTGCCGATGCCAGAGCTATTGCATGGCTTG
CAGGTAAAATTGTGCTCAGTGCTCTTATATTATCTTCGAGTCCACTCCTCTCATTGAAGGGATGTGAATG
GTTTGAGATAATTCAAGTTCAGGGCTTACTTTTGCAATAGTGACTGCTGGTACTTCTGGGCCAAAATTTC
TAGTCTTCCAAAATTGAAGTTTCACCATCAATGAGTGATTTCTATGTCTCAATTGATGTATAAATCTGTC
TAAACTCTATGCTTTTGATACCTGGTCTGACTTGTTATGATTTAAAATGCAGGATCAAGCAGTTGCTATT
GTTTCTGAACTGTGTGGTTCAAACAAATCAAAGCACATGAATCCTGAAATCAAATCAGTCTGTCTCTTAC
TACTACAAATAATGGAGATGGCCCTGTATTTGGAGCTCTGCGTTTTACAGATTTGTGGCATAAGACCTGT
ATTAGGTCGCGTGGAGGATTTTTCAAAGGAAGTGAAATTGTTATTAAAAGGTAATAGCCCCATCATGCTG
CCAACCATCGTTCAAATTTAATCTCTAGATTTACTATAATTGCCTTATTTTATTTTTTGCAGCCATGGAA
GGGCATACGTTCATAAAAGCATCAGTGACATCCTTGAAACACATAATATCTCTTGTCTATCCTGGATTGT
TGTTACAGACCGAAGGTTTCCTGTGAAGCACTTGTGGAAGAATAGGTAAACTTAGTTGGGTTGGTGACGG
ATTATGAAAATTGTGTACAGATATCAGATTTTGGCATGCCCAAGATTTTTGTAAGATTAGAAAGAAAATT
CTGCAAGCCATTATTAACTTGTGTACAATATCGGAACCATACAGCGTCGATTATCACCTAGATTTTCATG
GTCCCTCGATTTCAGCTGAGGAATATCACGCAAAAAACATGAAAATGGTCCCAAATATCATGAATCTTGC
TTTGTCCTGTAGTCATCTTCCTTTTGTGATTTAAAGGTATCATGTTGCACGATATGCACTGTTGTAATCA
CGTGGGGAAATGGATTGATCTAGGGGACTGTTTTTTCTCTTTTGTGCTTGGAAGGGACCCGCCCCCTTTG
CATGCGTGATGCCTCGAAGTAGAATGTATTTTCGGCGAGTCAATTGCTGTAAATACAAAGCTTTTGATTA
TACTGTCTTCGAAAAAATTACAGAAATTTATTATGTCTTGTCAAATATGTTTCTGTCTGCTACTAAAGCA
ACTAGAATTCCTGAGAAGAAATGTGTCTTTTTTTCTTTTTTATATGCCGATGGTAATGCTATTCATACTG
GGAATGATGGTCGCAGTGATTGGTTTCATTTTTTATTTAGAAATATATTAAAATAATATTTTAAAAAATT
AATTTTAATATTAATATATTAAAATAATTTAAATATATAGAAAAATTAAATTTACTCAACTTTTATTTAC
AACGAAATAATAACAAATGGGGAGTCTGGCGCAAACTGATATCCTTCAGACCAGATTTCTGCCACTGGCT
TCTTTGTAATTTCACAAAAAAAAAAAAAAAAAAAAGGGCCACTTGCATTGTCTCGTGTAACAAATGTTTC
TTAGAAGCTTCTACTTTTGAAAAAGGTGGCATTATTTTTTTTTGTTTAGGTAAATACAGTATCCCTTAAC
AATAGTATGGTTAGGTTGAATGTCGACAGGTTCCTCAGTGCTAGTAGGCCACCCTGCCTAATTATAGCTT
TCAGTTACCATGTTTATTGAATATTTCAATACTTTATTGACATGATTTTGATTTGTTTAACATTAAGATG
GAGGTAGTGTTTATTGAATATAAAGTATTTTGTATATATGTTAAAGAAAACACTAAAAATAATAATTTAA
ATCTGTAAAAAATATATATATATATTATTCAACCACACTCCCGTGATTTAATCACCTACCATTAGGATCA
TTAATGCACAATCTAATCTTACTGGGATTTGAAATCAAAATCAATGAGACTACCTTAAACAATCTCCACC
GTTAGATTATCTCAGATTTCTCTCCATCAATTCACGGTCCACCATTAATCATGGCCCATCATGCACGAAA
TTTGTACAATTTACCTATCCCCTCATAAATCCTAACGAAGACTTGCCATATCCAAACGAAACCACAGGCC
CCACCAAACGGCGCCGTTTCTACACGGCGCAAATAAAGCCAAACAGCACATGCCACTTAGGATCCCTCAC
TTAACCCTAGTCAAAACAAACCTAACCACAGTTGAAGTCCGACCACCTCGATCCCTCCACGTGTCCTGAT
CTAACGATCTAAATCCACCTTCTCTCCCCATAAAAACACCCCCATATCACTAAAAATCCTAAACAAATCA
CCCTCGACTTCATCTCACTTAGAAGGCTAATCCCTAATTTCTCTCTTTCATA
Sequence ID: 118
Sequence Length: 3093
Sequence Type: DNA
Organism: Poplar sp.
AAAAATTGATAAAAATTGTTTTTTTTATAATTTCACCCTTTTATTTTAAGTTGTTTAATAATTGAGTTTC
ATAATTTTATTTAATTTACTTTTACACGGGGATATTCTGTTATCACAGTCTAATCACATATTTGACGTGC
TAACCCGGATGGATTCGAGTTGAGTTTTTTTTTATTGTAATTTTTTTTATTTTATTTACCATCATTATAT
TTTTTTATTCTTGATATTAAATTATTCGAGTTTATTAAATCCAATCGAGTTAATGACTTATTTGAATTGA
CCTGCAATATAGCGAAGATCACCTATCCAGCACAAGTTAATAAAATGAAGGGAAAAACAATTTTACCATG
ATTTAAAATTAAAATTGTTGTTTAAAAACGAGAGGGGCTTGTTTTAGCACATTGAATGAAAATACTAAAC
GAACATCTGTAAATTTGATGGAATTTTTAACTGTGATTGAAAGTGAATGAATGAAGTGATTATATTTTAG
ACAAAATCGATAAAATAATAAGTTTTAAAACTCAATTGATAACGGATAACATTAAGTTAATTTCGGCTGA
TCTGTCAAAATCATAGGGATAATTTTGAAAAAAATAAAATGAAAAGCTCAATTGATTAAATATTTAAGAA
AAAAAGGTAAAAAATAGCACTGAATTAAATAAAAACTAAGTGAACTCGAGTCAACTAAATAAACTCACAA
TGCAAGTCATGCACGTCATCAGATGCAATAAGTTTTTTATATCATAAATTATTTTTCATTTAATTGTATG
ACGATAAAGCACATGGAAATTTTTTTGCCTGAAACAATTTTTTTAAAGGAAAAAAATTGTGATAATCATT
TATAACAAATGATAAAATTATATATTTTTTTAGTTTCGAGCTAGATAAAAAAATAGATCATTGATAATAA
AAGATGAATTTGTATTTTTTTAAATCAAGGAACAGAAAAAGACATTAAATAAAAAAAAACAAAGAAAAAC
AAATAGTAACTCGAATGCCTCATATTAACTCATTAAACTCGCGATCCAGTTCATTAACTCAATTAGGTTC
AATGATTTATCTTATCAATTTTAACCCAAATATAAAATAGAAAAACTTTAAAAATTAAAAAGAAAAAAAA
AGGAGCCCAGGCACGTGGGCCTGGTGGCCCAACGTGCATAGGTATTAAAAAATCATCTCAGCCTGCGTGC
CTCGACCTTAATTTTTTTAATTTTTATTTTTATTTTTTAAGGAAGCAGATGACTTGTCGCCAGTTTTAAT
TTTGTTGTTGTTGAAATAAAACAATAGATGATACATCGTTTGTTTTCTATAGAATCCGTGATTGGAGGTC
ACTAGAAAAATGGGGCATTGATTTTTTTTTGTCTTAGAAAATTATTTTCAACCCATTTTGACTGAAAAAT
ACCCTAAATACCTTAATAAATTTATACGTGGCTTTAAAAAAACCTTAAAAAATTCTCATGTTAGATCCGC
CTCCTCCGGCAAGGAGAAAAAAAAAAAACCATCTAGGTAAAACTCTTCTTATAAAATAGAAACCATTGAC
AACGATTTCAAGTTTTCAATCATCTTTATAACCAAAATCTTCGTCAACGATAATTTTCTCTATCTTCATG
GGAATCCGACGGTCCTCTCTCTTCTCTTAAAAAATAGATTGAAAATTGAGGGGAATGAACTTTGATTGTA
AAATTAAAATTATGACAACTAAAGGGATTGATCATCTATAAACTAAAAAACATGAGGGGTCAAAATAAAT
TTTTTATATGAAATTTGAAGCCATCACTTATTTAGTTATTTTAACCGGTCATTTTCATTTTGATCCTTTG
TTTTTCAATCCAATCATCTCTTTAAAAATATATAATTATGTGCTAATTTGAACTAAAAAAACTTAATTGC
ACAAAAATAAAGTTTAAGGACTAATAAAAAATAATTTACGAGAGAGTAAACAAAGCATCTCCAACAGATA
AAACACCACACGTTCTTAGTTTTTGTCAATGTAATGGGCGAGCAAGGTTTCATCACTCGTAAATGATGGC
ATCATTTAGAAGAATTAATTTTTTGTCAATAATAATAAAAATTGGCAAATGAACACAAATTATTATTATT
ATTTTTTGAAAAATAGCACTCACTTTGGTAATTTAGAAGAATTAATGTCCAAAGAACAGAGAGGAAAAGA
ACACGAGACAAACTAGGAAGCTTCCTTAGCACATGCCTTGTCTTCAGAATCTGTCAGATAAGGAGGGAAA
CAGGAGCGTGTGAACTCGAAAAAGAAAGTGATGGATCATGGACATGTTCCTCCCTTCTTGGATAAAAATT
TACCCTATCAAGCTAAACGACATCGTGTCTTGTCCATCCATTTTCTGCACATCTCTCTTTTCATCCTACC
AATAGGGATATGGAAACTGGTCCTCACGTGTTCGATTTTTTTATTTTCATATCACATCTATTATTTACTG
GATAATAAATTTAAATATTCATAAATTATTTATCCGTTTAGTTATAGGAATTCAATAAATATATATTATT
TAATAAAAAATAAAATAATATTTTTTTATATTTTTACATCTTTTTGATGTACTGATATAAAAAATAATAA
AAATATTATTTTAATGCATTTCTAAACAGAAAAACACTTTAAATCCAATCACCATCATAATTTCAAACAC
ACCTTTAGTGTTGACGTGGCATTGCCCATAACATAATCATATTGCCTAGCTCAACTTTATTTGAATATTT
TTGTAAATAAATAATTAAAAATTTATAGTACAAAAAATCGATTATCCTCCTACATAATAAGAAAAAAAAA
ATGGTAAGAAACAACAGAAAATCAAAATATAGAAACAAATAAAGGAAGAAAATGTGGGCTAGTCTGGGCT
GGGCTTGAGAAAACCCATCAAACAATTTCAAACCGGACCAGCCGATAGGGCTCGAGAAAAACCTATTTCT
TAACGTTAACATATTTTAGGGTTTGGGGGCTTCTTTTAACACTCTATTCTACTGAAAGCTGCCGCTGTTA
TATAAACAAAAAAATTATTTTCTTAGGGTTTGTGGCCTTGGAGAGATACTAGAGTTGAACAGAGCTGCTA
CTGCTCAGGAACC
Sequence ID: 119
Sequence Length: 3064
Sequence Type: DNA
Organism: Poplar sp.
TAGGATCCACAAGAACCAGATGTAGACAATGAAGAAGAGAAGAATAAAACCTGCAGAGAAGAGTTGTTAC
CATGCTAAGGTCAAACATTAAAAATTCACATAAAGGCACAAAAACAAGCTCTCCTGTTGATTGTTCAGAT
ACGTTGTTTGAGGTTAAAGGGTGATGATCATGGTACCTGGACATAACAATATCTTCAGAAAGCGAGTTGT
TGATTGCTAAGGCATCCATCACTTTCCTAAGCACCTAATCAACAACCACAAAACTTAAGACACTCATTTG
TACACAGACAATCAACAAGGCATAGGAATACACCTACCTCAATCGTAGATTGCCAAACATCAGCAGAAAG
AGAAGACCCCACTTTTTCAAACACTTCACCAAGCATAGTAAACATAACAGTATAAACCTCCCATAAACTA
CTATACTTAGACACTCCCTTTCCAGTAGCCCCAGCAGACCTTTGGTTCCCACTACTGTACGACGAGCTAA
CTAACTGTGCATGAGAGCAGTCAACAATTCGGAGAAGGAATTTCACTAACTGAACATTATCCGATAGCAA
ACATTTTTGCTGATATTCACTCACAAAATACCTACAAATTTTAAAAAAAAATAAAAATAAATAAATAAAT
GAAAAACATATAAAAATTTAGCAATCACCAAATGAAAAACTAACCTTACAGTCTCTATACATTCGACAGC
AAGACTAACATTAGCAGTACCAGCTTTTCCGAAAAAGAATTGCAGCATTTTCGCATACGAATCAAGTGCA
ACACTCCATGAATTAGAATTTAATTGCAGCGAAACGCGTTGCCTTTGGCATACATCATAAACCTAATTTA
CAAGATTCAAATTCAGTAAAAAAAAACTCACTAACACTAACAAAAATTAAAAATTTAAATTATTTTTACC
AAATGTGAAATGTGAGGGTACACTGAAGTGAAATCTTGTGATGAATTTGCCACCAATTCCAGTAAAAATA
TCAAATCAGAGGTAATCTGATCAAACAAAATGATTAGTAAGTTAATGCAAGAGTAATTTAGGAGTAAATC
AGTTGATTAGTGAGGATTGAGATTGATAAGTTACTTCGTGAGAAGGGAGATCGGAGGCGGCGGGGAGAAG
AGTGGGAGAGGAGAAGACTAAATCGTGAAGGAGTTGTGGAAGTGATTTTGATTCTGATTTTGGACTTCGA
GTTGTTAGGGTTTCGTCTCTTAGAGTGAGAAATGCTGTTCTCCAGGTTCGGATTATCGGCGGCGGCGGCG
GCGGCGGCGGCGGCGGCGGCGGCGGCGGCATAAAAGCAGAAATGATTTGTGGGCGGGGAAGAGGAGCTGG
GAGTGTTTATAGCATTTCTTGTGATGATTTAATTTAAATGAGTGCTAGTGTTTTAGAACTTGTGAATTGA
GGTGGCACTCTATTACAGCAAATATGATTAAAAATTTGATTTCTTGAAAATTAATTAGAAATCATATTTA
AAATTGAAAAGTAAAATATAAAACAAATATTGATTTTAAGATTAAAGATAATCTTAATTTAAGGTTGTTT
GGAAATGAGATTGTTTTTCAAAATATTTTTGTTTATAAAATATATGAAAATTATTTTTATTTTATTTTTA
AAAATTACATTTAAGTTTAGCTCAATAAAATTAAATATTTAGATATTTAAAAAAAATAGTTATAACAAGA
TTTCGATAAACCGCACTCCTAAAATTAATTAGCCTCCAATTTATTATTTGATTATCAATTGAATATCAAA
CTTGTGATTTATTAATTTTGTTACAATAATAAAAGATATTTAGTTTTTTTAAAAAAAAACTATAAACTCA
CGAGAACTTCAAATGCAAATGAATTTTCTCGGGAGAATTAAGATTTCGTTTATTTTTGCGTTTAAAATGT
GTTTTTAAAAAAAAAATTAATTTTTTTATTTTTTTACTTTAAATTAATATTTTTTGGTGTTTTTAGATCA
TTTTGATATGCTGATATCAAAAATATTTTTTTACTTTAAATTAATAAATTTTTTTTTTTACAAATTAGGT
TTTATAGGTCCACACACATGTTTTGGGGGTGAAAATAGATAGCATTTAGTTTTTTTATCAAAAATATTAA
ATCTTGATTTTTTATCATCTAAGACAAGTTTGATAGCTTAAAATTGAACCAAGTGACATCGCATCACTGG
ATAAACAGAAAATATATAGTCACTTATAATTTAAAAATATATACAAATGATATGTCATTTATTTAATTTA
TATATATAATTATAAATATATGCATAGTCTGTCAGACCAGACATGTGTGTTTTTATAAACCATGCTATTT
TTAAAACTTTATAATGTATTAACATGATAGCTCCTCTAAAACCTTTGTAGTGAAGACAATTATTTTATGA
AGGTTATCATAAATTCTCAAAAATATTCTGGAGTTTTATATATTTAATTTTCACCAATACCTCAAACAAC
ATAAATAAAATTATAAAAGGATAACAAAACTAAATTAGTATTGATATCTAATTTTTCTTATGCAAAAGTT
TCATGAATAAGTCGTGATTAATGTTTTTTATCACGAGTTGCATCATTATCACATCAAAATGAAAAAAATA
GTCAATATAATTATAAAAGGGGTAACTTGTCTGATTAAATTTTAAATTTACTTTCTAAAGATTACTAGCT
CGAGTCCTATAAATTTTAAGATCACTGAAGACTTATATGGTCATTTATTTAAGGGCTTGTGAGATTAGTC
GAATTACATGCCAGCTAAAATAATCAATGTTTTTATTATTTTTATTAATAATAATAAAAAAAGCAATCTA
TATGTTAAAATTCTAAAAAGAGAACATCAACGACATGAACAAATAAATTAAACTAAATAAAAAATGTTTG
AATATCAATAATGAAAGGCAGTAAATGTGAGCCGGGCTTGTACGGCCTTGGGTTGGATCTAAACCATCAC
CAAACAGGCCTGGTCATCTCCACACAGCCTTAATAATTATAGGGTTTATAAAGAAAATATGTCTAGGGTT
TTGGGGCAACCCTGAAACACAGAGAGCAGACTAGAGAGACGGCTGCCACAAACC
Sequence ID: 120
Sequence Length: 3014
Sequence Type: DNA
Organism: Poplar sp.
CTAGTACAAAGAGTAAAGCCTTAAGATTATAGATCCAAAGTTTGTAGATATGTATAATAGACTAGTGAGC
AGAAATATTTAATTTATCGTCACAAAGGCATGTTAAATAAAAGATGGCGGATAAAAGCCTAGAGTTTATA
CATATTTAAAACTATAGGGATGTAAAAGCATAACTAAAAACAAATGTGCTTTCAACACAAAAATAATAAT
ATAACTTTAGCATGCATACTAAGCATATATTCAAACTTGTAATATATATAAGACCGAAGTCCTAACATGC
ATACTAATAATAAAAATAATAATTTTTAAATTAAAATAAAAATGGTACTTACTTGTTAAAACACCTTAAA
TTAATGAATCTTTTATTGTTGTCAATGATGAATCATTTGTCCTTAAGGCTTTATTGTTCTTCACTTGTGC
AACTAGAATGTTTGCAATATGTCTTCCAAGATTCCAACAACATTATATTGGTTTAAAAGCACTTCTAAAC
AAGGTCTTCGAACCAAAGATTATGTAACTTAATATCTCTCAACAAGATGAACCTAAGCGCTAGATTTTAG
AGAAAAAAAAGCATAAAAAGGAATAAAAACACTAAAAATGAGGTAAGAAAAAGTGTGGAAAAATATAAAA
AATACAAGTTAAAAACTCTTTCTTTATCCTTTTTTTATAGTGAATTTTGAAAGTTAAAATATTAAAGAAT
TAATTATTGATGGATCGATGTATTTCATACTGAAAACCTTTTTTTTTCTCATAAAACCCATGACTAGAAG
GGACCAAGAGAAATAAGATCCATTATATAAGCTCACAGAGCCCAAAGCCGTGTAGCAAGAAGGTCTGGCA
AAATGCCAGGCCCAACACCCATTAGGCATAAGTCGCACGACTCCTTTGGGTCTGGCGAGATATCAGACCT
AACCCTTTAGGCCCGGCTACGCACTGTGCTCAAGCGCCTGCGGGTCTAGCGAGATGTTAGACCCAACCTC
ATTGGGCTCAACTACACCCCCGGTCCAAGCTCTTGTGGGTTTATAGAGATTTCAGACCCAACCCCCCTGG
GCTTGGATATGTACTAGAGCCAAGATGCTAGAATTAACACCTTTTTAAGCCAATAATTCCTCCGAATATG
AGCACCTAAGGTGAGAATCTAGGGTCCTCATCTCCTTCCACATTTTCTATGCAAAAATTCCTCTAAGGGT
AATCCATACGTCAATCAGTATTAATATTAATTAAAGGGTTGATCCCATCAATATTAATATTGTGTACATA
TTGATATATGATCGTTTTTTATAGACACGTGATACTCCCATCAATAATTAATGTAACGTAAGGAGATACT
TACAATCAATATTAATGTTAACTGTAAGGGTCTTCCCTCCCCCACTTGGATATGAAGAAGGAAAAGATTT
GAGTAAGAAAAAAGAAAATTCAAGGTTATGAATATTCTCTGAACGACTTAGAAAAGAGGTTTGTCATCTT
CACTCTTTTAGAACTTATTCTTAGCATTTATCATACATAAACTAAGAACATATTTTTTTGTTCTTAGAAT
TTAATGCTTATTTATTACAACTCCTCGTAATAAAGTTACTGACTTAATCATCAGAGGGTCTCTAAAATCC
TTAAAAGATACTGTTTTATAGGAAATCCAGCCATTGTCACCAACCCCTGAAATCCTCAGTTACAGAGAAT
AGAAGATGAACATAAACACGTGATCATTCATCATCCAAACACGGTGAAAAATAGGTTCTTGACATTCTAG
ATTTTAGACCCATAATTTATTTATTTAAAACAAGTTTCCAACATCTTCATTGCAATCCCGGGTATAGTAA
ACATGAGTTCTGTTCCTCGTATCGAAAACGGCATGACCAAAACTGGCCTCACGATTTGCTGAGTGATATG
GCCGTGGACGTGCCAAGCTGCATCACCAGATCAATACATTACATTTAACAGGCATATATCTATATGAATC
TTGATTTATATTTGTTGGTTCAATCGAGGAATTTGCATAGGAAGAAAAGCCTAAAAGAAAAGGAAAAAAA
TCAATGATCTATTTACTTGTTTACCAGGCCTCCGAGATGCCCTCCATCACTAATGGACAATGGCGGTGTA
ACACAGGAGCCGATTGATTCTTTTTTTACAGAGCAATCTCCGTTTGTAATGTTGTATGCAATGTCGAACA
AAAACAGGCTCCAAGCTACTGCCATGGACTCCCCTTCCGCGTAGCGAGAGCATATTACCTTCCACAGAAT
GCTACGAGCAAAACAACATTCAGCCATGGCGTCTATAAACTTTGGTACGTAGCTCCTTGTCAAGCCTTTG
TGTGTTGTTAATGCTTTTTTAAAATTAATTTTTAATTAAAATTAAGAAAAAAGGTTTTCATAAGAAGTTA
TAGCAAAGGAGCCCAAAGACAACATGTAAGCAAACATTCAGCCCAGCCCACTAAAACTTTCCCCTGTACT
AATGGGCCTCTGTAGCCCATTTTCACACACTTTCCAACAAGTAGCAGAAACAGAAATAACTTCCCAGTCG
GAAACCGCCAGTCCAAGAAGAAGAACCTACCATTCAAAAGTCAAACGGTTCACCTCTACCACGTGTCCTT
CTCCTATTGCCTCTTCCTTTCCTTTCTCCCAAAGCAACAAAAGGCCCATACCCACCGTTACGACTGTTAG
ATAAGCTCCACCCACCCCCTATTTTACTACCATTCATGGTCCTTAAAGTTTCGACTCCTGGCGACATTAA
GCCCTTTACTTTATAGAGGAGCCTTCTCTATGACTTTCTAGAATCTAGGTGATTTCTTGTGGGAAAATAA
CCTAAGGAAGAGAGACAAATTGGACTAAATAATAAAAATTTATTCTTGAAGGATGATATATGTAATTATA
GGTATAAATAACCACGCAATCCATTCGTATATTTATACCTATAATAAATAATGATAATAATAATAATAAA
AAAAAAAAAAACGAAAACGAATCTTTTTTCTATATAAAATCTCTCTCTCGGTGAGTGTTTGCCCTCATTT
AACA
Sequence ID: 121
Sequence Length: 3020
Sequence Type: DNA
Organism: Poplar sp.
TTCGTTATATTTCATCAATTTAACCTTGGAAGGATTGGATTTTAAACAATTTAGTCTACTATACAAGGTA
TGTTCATCACGGTGTATTAAAAATCCGGTTTGATTTGGTTGATTTGATGAAATATGACGAATATAAGACA
AGATTGATGAATTGTTTATTGAAGGGCCTGATCTGCTGAATTGAAACAAATACAGGGACTAAACTGTGTA
TTTTCATTTGAGATAATCTTATTTCCACTCTCATTTATGGCTTGTGGTCCACACGCAGAACCAAGGGCAG
AGAAGGCACACAGAATGGACAGCCACTAGCGTGAATTGGTGAAAAAAGGTGCGCCGGCAGTGGACCAGCA
TGGTCCGTTGACTTCCTGACGTTAAAACGACTGGACTACGTTCATCAGACTCCTCCATGCCACGAATACC
ACCCCACCTAGCCAGTGCTTACTGCCTTCTTTATGCCTACCTCTCTCGAGTTTTTTTTTTTAAAAAATTA
ATCATTTAAATATTTTTTTTTTGAAAAAATAGCACAGTGTATAGAATATACAGATAACTATGACAGTTTA
GTTCAGAGAGTAGCAATCGAATTTCTCAAATTGTAAAATGATCTTATATAATTTATAATAAATAAAAAAA
TATCTAACTCCAATGATGATATTATCAATATTATTAAAAAAAACTTCAATGATATAAATTTAATAACGTT
AAAAATATTTAAAAAAATCATTAATGATGGAGAAAGAAAATTATATGTATTATTCAAAGCTAGTAAAATT
TATTTACTTTTAAGTAATTAATGTGGTCCATTTTAATCATTATCGATAATTTTTTTTTATATTATTAAAT
TTATTTTATCAAAATCTTTATAATAATTAATACCAGAATCATCATGATCGGAACTTCTTATTCCTTTTTC
TCCGTGAAGCCAACTTTACTCTCTATGACAGTATCATGTTTATGTACGGGCTCTCAACGTTTTAAACGAT
GAAAGGACAAGCCAATTATTCCTTCCTCTTCAAAATCTGTTTCTTTGTCGAATGTTTCCCATGTAAGAAA
CGTAACATGTATAACCGATATATAAGTTAGCAACTAAGACACATAATTAAAACCAGTGGTGCACGTTTGG
TAAAACCCACCGAATAAACGTAAGCCCTTCAAAAATTGTCTCAACCCCTGTTCGGAGAAAACCAAGAACA
TGAAGCTTTTGGGTTTTAAAACAGCTCGTGAATATACACATATCATGACATAAAAATGTTCACAGAGACT
TGTGCAATAGCGTTTTTTATTCTTCTTCTACTTTTGTGCAGTACTGAGAAACAACAGCGAGCTTCAAGTT
GCGGGAGCAAAAGTAAACAAAAATAACTATGACCATACATTATTATACCATACCAGTACTACGTACGTAC
AGGCTACATTATTAGGAGGGGAGTGAATACATAGAGTACTGTGCACGGGGAGCATGGTAACCTGGAGGAA
CTTGGAAAACATGACTATGGAGAGCCATATCAACGCCCTTTTTCACTGCCCTAATGAAACTGCTTGTTTT
GCCTCCGCTACACGGCAATGCCATATCCTAAACTAGAAGAAACAATGAAGAAGAAGAAGAAGAGCCCGGT
ACACCCATCTTGGTCTGCTTCCCTCGCTTCACCACTTTAATTTATTAGAGCCCAGTAACCATTTTTAGCT
CATTAGGCTGCATTTCTGCTGTTCTTAAATATATCTTATCAGCCCTTTTATTGTAATCTAAACCAAAGGC
CCAACAGCCATACCGAGCTCTTCGAGGTTCAGGCCGCGCATTTCGGATGTTCATAAAAACTTTGGCCCTG
ACACTGTAATCCAGAGTAAGTCCCCAAGGTTCTAAACCCAGTGAACAATTTAACTATATTAAGTAACATG
TGATTTGAGTAAAGTATTACTAAATTCCAGCACCATACTCTTTTGATGATGTCTCATGCAGTAAATGCAA
TAGCTCCTCGTATTGTTTCAAGCTTGAGTCTAGAGAAAGTTCAATTTTTTTTTTATTGTAAATACAGGTG
TGGGTATTTTATCTTAGTTCTTGATCAAGAATTAAACATAATTAGAGTAGACCAAGGTTGATGTCATTAT
ACATCAACTAAACAAATCTTAAAGGCTAATCGATAATTAACAAGGATTATCCTATTTCATAATTCAATCA
ATTTAAACTTGGAGGCCAGCATTTATGAACAATTAAGAATATTTACATACAAAAAAGCTTAACCTTGATT
GAAATTAAAATACATCCACGAGTACCTTTATTGTAAAAAACAAAATTATATAAAACACGTGTAAACAAAA
CCCAGCCCAGCCCATATCAGCGACTTTCCTGGGCCAAAAGCCCATTCCCAAACTCCACGACAAACACACT
TTTTATACCCAAATATATCCAGGACGTAACTGAAGAAACATAGATTAGCTTCCCACCAAAAAGCCGAACC
TACCATTCAATCCAGCACTCCACTGCTACCACGTGTCCTTCTGCTATTGCCCATCCGCTGCCCTTTCTCC
TTAAGCAAACTCAAAAGGCAAAACCCACGCTACCGTCTGTTAGCTTTAAAGCACCGCCCATCTCCCTCTC
TACGTATATTAGTACAATCGTATTTAGTATTTATACCATATGATTTGGAACCAAAACCATCCATGGTCCT
TAAAGTTTCAATATATACCCATGGATCAAAATAATTCAATAATAAAAGAGGTAAAATCAAGAACTGGCCC
AATCCATCGCCGATCTTACTCCTTTAAAGTCTCAATAAATTAACTACAATTATCCGCCGTAGGGCCCACC
CTATAAAATTAAAAGTAGAGGGTCGAATTTGATCTAATCTAAACTAGAGACACGGATATTGATACACGAG
GGATGATATAATGAATCCACGTAATCTGTATCCCAATCCGTATCGGATTCGTATATTATACGTCTAACAA
TAACCAACAAACAAGTATATTTTTCTATAAAAACCTCTCCTTTGCCAAGGCATCGCCATTACCTGATTTT
CAATCCAACA
Sequence ID: 122
Sequence Length: 3038
Sequence Type: DNA
Organism: Poplar sp.
ACTTAAACTTATTATGAAATTGTTTACACGAGTATTTATTCAAGTGTGTCCGCAAATTACTAGTGTTACA
AGTCTTTGAAGCACATGCATAATTTTATTAATAGTACTCGATCGCATGTAGCTCTCCTCGACAATGGATC
GTAATCCTCTCTATATATTATATCAAATTGATGCTAAACTTCAGATATAAATATAATTATAAAGGCCACG
TTCCTTTTCATGTTACCTTTTCTGGGCAATAACACTTGCATGAGTAGCTGAAACAAGCCTGGTCTCTATT
CTAGCATCATCATCATTTGTTGAAGGTCGAACTTCGTCATTCGATGGATGAGCACTTCGTTGATCAACAT
TAATTTCTTGAATGGAATTGAAGAACTTGTATTCTTCATCCTGAACAATAAGATTTAATCAATTTTAGTT
AAATATTTATACAAAAAAAGACGTAATAATTACTAGTTCGGAGCTAAGTCTAAGCTTATTAATTTTGCAG
AAAATGCAAAAAGAAAAAAAAAAAGAGCAGTCAAACAACAAAACTGAGATTGACCAGAGTATTGTGAAGG
TTAACTTGTCAAAATCAAGCTAGGGTTTCTGGTCATGTAAATCTCTAGCTAACTTCAAAGCTTTTGCTGC
TTCACAATGGACGAGGATGGGACACATGCACACACAACTTTACGCCGTAATCTCGTGGATCCATGCCTTT
GTTTTGTGGATGTAGCATGAAATATATTTTCAAGACGATAACGTTTTTGTTAGTGGTGATTAATTTTTTT
TCGGTTTAGTTTTTATAAAAAAAAAAAATTAAACTAAAATTTTTTTAAAAAAACTAAAACCGAACCGAAA
TCGGGTCAAACCGACCGGTTTCGGTTCAGTTCTATTTTTTTAGGGTAGAAACCGGTTTGACTCGGTTTTT
TCCGGTTTTGGCTTGGTTTTTTCGGTTTAGCTCAGTTTTGGCTTGGTTTTTCCGGTTTGGCTCAATTTTT
TTTGAGTTTTGGTTTTTTCATATATTTTTTTTCAATTTTCTCGGTTTTTTAGTTTTTTTGCTCACTTGTA
GTTTATGTGTGAGAGTTCTCTTTGTATTTTCATTTTTATTATTGTGAATACAAATCTGAAATACATACAA
ATTTAACTATCCCAAACAATCGGCGAAGACGGATCTATAACAAGTGCAAATGGGGCCGCCCAAAACTGAG
AACAATTCATAAAGCTTGATGGATTTTTTGTTCTCTCCGCAAACATGTTGGAGTTTATCTAATTCCAAAG
AGATAATGAGTAAATTAAATGATGAACATTGTAATCATAAAAAGAAGAAGAAGGAAAGATATAATTATAA
ATAAACTTCCCAGCATAATCATTGATATTGATTTGGTAGTTAATTAAGAAAATATTTCTTGACTTTTTAA
AATTAAATCTCTTCTAAACCATTGTATAATCTGAGGGTGTGTTATATATTAAAATCATCTTATTCTGGTT
AATATAAAATGATCCGAAAATACTTTTATAGTAAAAAAAATTGTTAAAATTTTGCAATATCTTGATTCAA
CCATGTATAACAAAACATGATTACAATTTTTGCTTGATCTTTCCTTGAATTGCTCTGGTTGGATAACATT
AATTTCGTGTTTTGAAATTCTCAATAGCATCTCTAGAACATGGATTCACGGTGACAAAAAAATAAAAAAA
TCAAAATTGAAACCTGGAAAAAATATTACAATCACATGAATTCACCAGCAACGACAACACAAGAAGGGAA
CCGGTAACTTGTTAGCGGCTGTTTGACAAACATACTTGTCCATGTATTTAAGTATGTTTTAAGAGTAATC
TATATTAAAAAGCATTAAATTAATATATATTTTTAGTGTTTTTTAATAGTTTTGATGCTGATAATAAAAT
AAAAATATATAAAATAATATTTTAATATATTTTTAAATAAAAAAATATTTATAAAAAACAACATCATAGT
ATCAAACAGATATTAATATCAAACAAACATTTTATTTTTTTTGGTGTGTTTGAGATTAAGGTGCGGTTTG
ATTTTTGTAGAAGAACTCATAAAAAAAAAAAAAAAACATGTATTTCTAACTTTTAGGAAGATATAATTTA
TACTTTAAATGATTGTACTTACAAAAGAAATTAATCACACTCCACCATATCTCAATTTCAAAAATATACT
TGGGCAATGTTTGATATTAGTTATTTTTTAAAATATTTATTATTTAAAAATATATTAAAATAATATTTTT
TTATTTTTAAAATTTTACTTTTAATATTAATACACCATATCAATTAAAAAATATAAAAAATTATTATTTT
TTAAAAACAAGCACAAACTTTCCTTCTTCCCCTTTCTCTACTAGAATCGTCACAGCTTACGTATGTTTCC
CTGACCCTAATAAATAATAATAAATATCTTTGAAAATTAGTAAATAAATATTAATAACGGTCACTATTGC
GGGCCCAAGCACGAAAAAGACAAGCATCAAAGAAACACAAGCAACAGAATCAAAGGCCCACTCAGTCCGA
GACCATGGAGTGCACGCTTATTTGGAAAACTCAATTAGCCGAGCATTAATAACGATCACCTTCACTTCAC
AAAAGCCAAAAAGTCAATTTATTACGATGGAGAGGATTGTGATCGAGGCAAGGATGGCGTAGCTCCTATT
TTTAAATATTTTAAAAAATATATTCGATTTCAAAATTATTTTCTAGTATATCAAAATAATCTTAAACTAT
ATATAAAAAATAATCTTAAACATTTTTTTAAAATTTTTTCAGTAAACACCATTCAAACGGCGCCTAACCA
ATATATATATATATATATATATATATATATATATATATATATATATATATATATATTTATTTATTTATTT
ATTTATTTTTTCTTTCTGTTTCTTAATCCATCAACTTGCTCACGTAACTATACGTGTCACGTTCACAGTG
GTAATCACCACACAATATTCTGGGAACATCGCCGCTTACAAATATCTCCTACGCTTCCAGTTCCCACTGC
ACAGGCATTTCTGTCCCAAGTCCAAACC
Sequence ID: 123
Sequence Length: 1129
Sequence Type: DNA
Organism: Poplar sp.
AGACAATGATGTGCAAATAAATTTGATGTACTGCTCGAGCGAAAGCCTCGGAGGCGAAATTAAGGTTGTGTGAT
GGATATTTCACAAGGAGCATTGTACTAATTTCATCAACTCCACTCATTACTATGCTTCGAATTTGTAGATAGAT
GCGAATCAATGTCACAAACTATAAACCCAATGAACCTTATAAGAGGACACGTTTTTTCTTCTCCATATAACATG
GAGCCATGTGCCTTTCTATTTCTTCAAGTACACTTCACAAGTGTTCTGTGGCCTAGTATTTCACGAACCGCATC
GCTCTTTCTTTAGCGCTTCAATCATCAATGGCTGCAAATTTATACTTTCTTTAATTACCATCATGGTTATAAAA
TTCAATGCAGAAGGATTCTATCGATGTGACACTTAGGTTATAGATCATAGGGTTAATAAATCTTTTTTTTTTAA
ATAATAAATTAAAATGAGATATTCTTGATTAATTCTATACTGAATTTTTAATTGAATCAAACTGAGTTAATATT
CTATATTATGAGTTGCGCAAATCTAAAACGACTGTAGGGCTGGTCCATATTTTAGAACTGGACTGTATCTAAGG
CAAACCAAAGAACGAATTAATACAAATATAATTCCCCCCAAAACTTGAGACTACAAAAAATGGGGTCCAAAACA
TTATAACGTGACTTGGTAAGAATGGGGTCCAAAAGGTGACCCAAAATGAAATAAAAAATTCGGGAGAAAAAAGG
GAAGAAATAAAGAAGACCGGAAATAACCGTTGTGGGCTTGACTTTGGGTTGATGCCAGGCTGACCCCTTGAGTC
CTACCCTACACGATAGCTAACCCCTCACCCATAGAAAAGAGAAGATGATTAGCCATCACATGGCCTATCTGCAC
ACCTGGTCCCACCCCCATTTTCTCTCTCTAGGCTCTCCCAGGTACAACAGCAAAACCCTAAAATACCCCTGTCT
TATCCAAGATATGCAACCCACCTCTCCCTGCTTTAAAATGACTGACCACCTAGGGCACTTACAGAGACACCGTC
GCAAACCAGTAGCTAGAGAGATAGAGAGAGGAGGAAAGGCTTGTGGCAAGAGAGCTAATTAACTGTAAAGACAG
AGAAACTCCTTAGATCAAA
Sequence ID: 124
Sequence Length: 3102
Sequence Type: DNA
Organism: Poplar sp.
TGATGCTTGGTTTGTGCTTTTGTAACAACTGCCCATGTATAAAATCAATCTGTTGTTCATGTATTAATTATCCC
ACGAATCAATCGGCTGGTGGTGGCCGGATTATGCATGATGTCATCGCAAAATCAAATCTTGCTTTTGATATTGC
AAGAACCACCGTAGCAAACCACATTTATGATGATACCAGGCTAATCAAAACTCTCTCTCTCTTCCTCGTTGCTT
TCGTTGGCTACGTGGGCAACAAGCTAACTGGCTGGCTGGGCCACAGAAATGCCACCATTATATTTCAAAAAAAT
TAAAAATGAAGCGACCGTTGGGGGCGTCGGGATCTTGAGAAGGGTAGACAAGTCCCTCTCCTTCACATTCCCCA
ACGGCTATAAGATTGTAAGAAGGCTCTGGAACAATCTAGCCGTTTTGGGATACCGGACACGTGTCGTGGCTAGA
ATGGTGATGATATGTACAAGTGAATTGAGTCGTTTGATACAAAATGTCTGGTCCTTTTCTTTTGTTTGGCATTT
ATTTATTATTCAATAGAATATTACGTTAGCATTTTATTTTCCCAACTAAATAAATGTTCGGGACAGTTAATGGT
GTGTGTTTAAGGCGATGTTGTAAACTCGGAATGTTCTTGTAGGCTGACTCGTGGTCAAAATTGATCTGGTTAAA
ATAATAATAATAAAAAAAAATCTTGACTGGCTTAATTAAAAAACTAGATTAACTTACTAATTCCATCAAAAAAA
AAAATTTTATTAAGATAGTGACTTGAGTTTGTGTTTCCTAATAAAATCAAATCATATCTCACATAAAAAGAATT
AATTTAAATTGACTCGAGTTAACTCATTTAAAAAAAAATCAAATAAAATTATTTTTATAAAAAAAATCAATAAA
TTTTTAATCAGGTTTTATCCAAATCACAAGTCAATTCAGGTTTTTTATTAAGTTAAACTAAGCTAATATTTTCT
ATTTTTTCTTCGGCCCCGGACTGGTCTAAACAGGTTTTTCAACATGGGAGTTACATCACAAGATTAAATGTACA
TTTCAAATTATTATGACTCAGTGAAATCAAAATGAAGCGAGGGAGGGAGGGAGGGAGGAGATAAGACTGCATTC
CGGCGAGTGCTTGTGATGGCGTCATTTAAAATACTGCCTTTGATAGGTAAGGAGAGGCCCATCAAAAACACCAC
TACCCACAGCAAAGCCACTTCGGCAATCATTCTACCTCATAAAGGACTTATCTTTTTATCCTCTCTTTTCCTTT
TGGATTAATAAATAATGAAAAGAAACACCCTTCTTCTACAATTTACACGTACTGATTGCACTAAGCATTTGTTT
TGTTTTTAGAAAATGACATTAGGCTTTTTTTGTTTGGTAAACTTAGTCATGGAATATCAAAAATTATATGAAAA
TACCTATAAAACAAAAAAGATTAAGCATATTTTTATCAAAATTATTCAAATAACGGTGCACATACCCTAGGAAA
CTTTACTTTTTTTATCATACGAAAAAAAATTATACTTTTATTGCATGTAGAATTTTATTATCATGTCATTTTTT
TCGACATTGCAGTATTCCTGAAATTTAAATTGGGACTTTATTTATTATTGTGACCATGATTGTTTTTAAATGTA
TTTTTTATTTGAAAATATATCAAAATAATTTTTTTATTTGTTTTTAAATATATTTTTAATATCAACGTATCAAA
ACGATATGAATACATAAAAAAATTAATTTAAAGTAAAAAAAATAAAATAAATTTTGTTTTAAAACCGTTTTTAA
AACAGAACAAACGAGATCGAAGTCATTCTTCACATATAAGATTTGAGACAAATCATTTATACATGGACTGGCTT
GTTCACATGGTTTTTTTCCACAACCTCTACTTGAATCCAAATTTGAGACAAAAGACGTGAAACCAACTACTCTT
TACCATAGATAGAGCCTTCAGAATTGAAGGGTTGTGTAATTGATACTTGATCGATGATTAACGTTGTTCTAATT
TCATTACCTATTTTTCTAACCCAAGCATCATTGTCGTTCAAATATTTGTATAGATGGGAATTATTGTCACAAAT
ATAACCCTAATGGATCATAAAAGAGGACTCGTTTGTTCTTCTCCATATACTATGGAGCCATGTTCCTTCCTGCA
TTTCAAGCACGTTCATGGCAGAAATTAATGCTGATGGGGCGCCTAATGTGTTCATGGACTGCATTTCACACTTT
CATAGCATTTCTATGGCAGCAAATCTATAACCATTTGAGGCAAAGACATACTCTAATTCTCACTACCGAACTAA
TTAACTACACCCAATGCAAAATAAACAACGAACCAATACAAATATAAGTTCGGTTTGGTTTCATAGATTGATCC
GAAAGATTTAGTATCCTAAAACTTAATCTGGGTTAGGTTTTTAATTGACGTAATCAAGTTATTAGTAACACACG
GTTAAGACAGTTAAATCTGGTTAAATCAACATCAAGAAAAACCTTATAATTAATTCAATGATATGGGATTTAAT
CTGATAGCCTAACAATCATTTTTCCTTTCAAATCAGCCCTCCAATCGTGTACAATAATCATGTCCAAAACATTA
TACCGTGACTGTGTAAGAACTGGGTCCAGAAGGGTGTGCGAAATGAAGACAAGCTGGGGACTGGAGAAAAATAA
AACGAAAATAAGAGTTTCAAGTTCGAGAGAAAAAGCAAGGAACGAAAGAGGACAAGAGATGACCGTTGGTCGCG
TGGCTTTGGTTGGATGCCAGGCTGACCCATTGAGTCCCCATAGCTAAACCTTCTCACCCTGTATAAAAAAGGAG
ATGATGAGCCATCACATGGCCTATCTGTACAACTGGTCCCACCCCTGTTTCCTCTCTCTACAACCCATCCTCTC
TGTCTCTCTACCCTCTCCTAGGTCCAGCAGCAAACATTGTTTTGTTCCCAAAAGGCCCCTGTCTTATCCAGAAC
ACGCAACATTTTCATACCCTTCTCTTCCTTCTTTAAAAGGACTGACCACCTTAGGCCACTCACACAGTTACAAG
CCGAGAGAGACGCAAAAGCTTGTGACAAGAGAATTAATTGTACCGAGAAAGATCTTTAGATCAAAAAA
Sequence ID: 125
Sequence Length: 2221
Sequence Type: DNA
Organism: Poplar sp.
CAATTCTAATTAGGTAGATGGAAAAACAACGTTGTTAAATGAAAAAACATGCTGATTTCTAGTCTTCCCC
AAGCCATCTTTAGCGTTGGTTTTCACCATTAAATCAGCCCCCAATCAAACTGAAATCTCATGATCATAGT
CCTGTTTTATTCCTATAAAAAAAACAAAGATCGAGAGTCTAAAGACAAGTAAGTAGAAGGTCTCAGCAGC
GAAGGAGACAATTAAGATGCAAGGATCATGGCATGCATATCTTAACCTGCATATCATGTCTACAAATATT
GCATTTTCTACTTACGAAGATCATTGGACACAATTGCGAGTAATCGAGGAAGAAGAAGAAATAATTTGGG
GGAAGAAATGCAAAGAAGAAGAATGTGAATAGAGGAATTCATGGGCACAAGTCCCTAGACAACCCCACTA
TTCCAATTGATTCCGCATGCTCTGGCAGTCCCTAGACAACCCACAATCACCTTGCACGGTCCATCTCAGC
CGACATCAAGATGCGGTAAAATAAAACTCATATATATCAACTAAAAGAACTAAAAAAATTGAAAGCTCAT
TAAAGTTTTATATTATAGGTTTTTAATTTTTAAAAATTATTTTTATAATTAATATATCAAAATAATATAA
AAACAATTAATTTTTAATAAAAAAAATTAAATTTTTAAAAAAACGCGGCGCAACGCGTTTTCAAAACGCT
CACGAAGGACACACACTGAACACTCTCCTTTCATTATTATCTGTCTTTTTTCTGACACACTCTTTCACTT
ATTCTTCTTTCTTCTCACTCGCTGCCCACCACACGAAATGAAGGAACCACACACACACCCACACATGGCT
TTGCGATGTCGGTAGGAGAAATTTCCAGCTTACATTATTTGTTACGTGTCTTGTCTTTGTGCTGGTAGTT
TTTTTAAAGTGTTTTTTATTTTAAAATAATATTTTTTATTTTTTAAAAATTATTTTAACATCATATTAAA
ATGATTTTTAAATATAAAAAAATAAAACAAATTTATATTTTTTAAAAAATATTTTTAAAACCTAATAATA
AACACACCACCGGCCATAATTGAGCTAAGATATTTTCTAGTGGAATTATGGACAAATATTTCTACATAAT
CCAAAGATGGCAGGCGGTGATAAAATTCTGGATTGATCTTCAATAGATTTCACACCTAAGATGATCAAAG
CAACCTTTCTTTGTAACCTAATTGGGATTTACATTTTAGATAATAAATTAATTGACAAACAATCAGTTCT
TAGTTAAAGCAAGTGTATGTGTCTGTTTGGTGTCGAGTTGGAACAACGGTTTTTTATCCATTTTAAAAAT
ATATTTGACTTGAAAAAATATTAAATTAATATTTTTTTTATTTTTTTTATAATTTTTACATGTTGATGTT
GCAAATAAAAAAAACTTTAAAAAATTATTACTTTAATTATTTTGGCAAACAAACAAAGATCATAAAAAGA
CTCGTTTTCCTACTCTTTTCTTATATTTTAGAGTTTAGGTTTGTTTCTTGTAAATACATTCTTTTAGTTT
TATTTTAAAGATATTATATGATTAATTAATATACATACTAAAATTTTATTTAGATATCAATTATAAGTTT
AATATATATTCAATTTGAACATGCTTGCTAGAATCTATGTATATATAATTTTTTTTTTATCATGATATAA
GTCAAGCTTGTTTGGCAGTGTGGTTGCGGTTGCTTTTCAAATAGCTTTTCGTGTTAAAATGCATGTCAAT
GATATTTTTTTTTATTTTTAAAAAATTATTTTTAACATCAGCACATCAAAATGATATGAAAATACTAAAA
ATATATTAATTTAAAGTTAATAAAAAATGAATTTTTTTTTAAACGCAGAAACAAGCAGATCAAAATATTT
AAATTATAAAATAAACATGTTTTCTTCTCAATCATTGTCATAAGATTTGGACAACATTGTATGTATAAAA
TGTGGGTGTCTTGGGCTCAAGATCTTGGATCCCTCAATCATGATCCTTTCTCAATTGAATCTGTTAAAAA
ATAACCGAAATAATTTATTATTATTATTTTTAACAAACGAAGAAAACTAGCACATGAATTATTCACAACC
CCCCTGGTCGAAGGGCCCCATTATCTCAACCTCAATAACAGATATTATTCCGAGCCATCTATAGCTTGAC
ACGTGTCATTGCACGTGTCAGCAATGGATTGGTTTAAACCAGTATAAATAA
Sequence ID: 126
Sequence Length: 3095
Sequence Type: DNA
Organism: Poplar sp.
GAGGGGAAACTGCCGGGGGTCCTTGAGGTCTAATCCTCTTGCCCCTTGCCCCTTGAATCTGTGATGTTGT
CCTGCTTCTTCAGAGAACAGGGGAATGGCGAGAGAGAACCGAGAGAGAACAGGGGAAGAGAAAGAGAGCC
GAGAGAGAACAGCCACGAGAGAACTGGGTATGCAGGAGGGGAAACCGAATGAGGGGAAATCCAGGGGAAT
GAGGGGAATGATAGGGGAATTTGAGAAACCAAATGAGAGGAATGACAGGGGAATGAGGGGAAACCAAATG
ACAGTGGAATGAGAGGGGAATGAGGGGAAATCGAATGAACTGGGGAATGATAGGGGAAATGAGGGGAAAC
CGAATGAGGGGAAACCGAATGAGGGGAATGACAGGGGAATGACAGTGGAATGAGGAGAATTTATGAGAAA
CCGGAATGAGAGGGGAATGACAGTGGAATGAGAGAATTCTTCATACAAAAAAAAAAAACCCAAATATATA
ACTCTTACCATAAACTAATTTGGCTTTCTTCATATAAAAAAAAACTTATTTTAAGCTTTTAAAATTGAAA
AAAAATATATTAATAGGTTTAACTTGAAAAATATTTCACAAGCTTATTTCTCTATAATATGATATGACAT
ATATAGTTATATTTTATAAAATAAGCTATGTAAGAATATAAGATATTATAAAAAAAATTCATCATTATAC
TTTTTAGATTTCATTCTTTTATTGCAAGTGATTAAATATTTATATTAAATATTTTATATATATTAGATAA
ACTTGAACAAAAAATTAATTCATTAATAAATTATTTTCCAGTTCATTTTCCATAACATAACCAAACACTG
GAAAGTGTTTTCCAGTTTATTTTTCATAATACTACCAAACATCAGAAAATACTTTTCCGGAATTCACTTT
CCAAAAGAAAACAACTTTCCTGCAAACAAACAGAGCATAAGAATAGAAATTGAAATTACTAAATATTTAA
AAAGATAGTAGTGAAAAAAAATAATTTGCTTTTTTTTGTTTTTTCCCCTTATGCACCTCTTTTTTTTAAA
GCTCTCTCATCAAGAGAGAATTGCCATCCAGATAGAGAGAAAAAGATACTATTCTAAAAATAACTCAAAT
AATTTTTTGAGTTACTTTAATGCCTTGCAACTGGAAGAAGACATGATGACACGAATCATCACACGTACCC
ATTATAGGCAAAGGCTGGATTACATCTTGGTCCTCAAAGTCTATTATTGTGTGATAAGGTCCTACCAACC
AAAAAGAAATGCCTTCTTTTACTACCCTCTTGCCCTTAAAGTCTTGCTATTTTTACCAAAGTTTAAAAAT
ATGAAGATATTGACTTTTGCATTACTGAACCTAAAAGTTATTCATGCGAGGATTATAAGGTATGTAAAAC
AATATGGTTCAATTTTTATAGATGATGTGAGTAAATTAATGAATTATAGAGGACAATAGATGAGCCGCTC
GATAAAACTACAAGAGTTAGAGCATCCAATCCAAATATCCAAGAAAATAAAGTTATATGTATATATATAT
ACATATGCGTGTGTGTGAGAGAGAGAGTGAGAGAGAGAAAATTATTATTTCAACTAGATAACTTGACTCG
AAATTTTAAAAAATATTTTTATCTATATACTATGTTGATATTATATAGATAATACATTTATATTTGTCGA
TATTATAAAATAAGAATAGAAATGTTAGATATATCATCGATAGAAAAAAGCCAGAATTTTTGTTAATTAA
CACGAAAACTAAATGAAGTTAAAATATTTAATTGTTGAGGTCCTAAGAGAATATCCAATCTAATTGTTTT
TTTCTTTCAAATTGAGAAAACAATGAATCCAATGTATTTCTTTCATAACAATACAAGGATATTTGGAAGT
TACAATATTTATAAAACTTTTAATTTACAAACCCACACCAATTTTTGTAGGCGCATTAACTTTTATTTTA
AAAATACTACTCCGAGAAAATACTCATCAAAGACCCCTCAATAACTGAATTCCCAATAAAAAGGGACTAG
TTTGAGGTTTATGTAATCAAATTAACACTAATTAATATAATAAAAAAGAGATTATGATAAAACATGTAAG
ATAATTTTAATATGTAAAGCATATAAACCCAGTAAAAAATTAATATGATACATATCTCATGTTATTATTA
ATTAAGTGTTGAAGTAATTATCTAAATAAATCTTTATTTTGTAAAGAATAAAAAGTCTTCTATTCCATGT
CAAATTTTTAGGGTTATCTTATGTTGATGAAAAATATAGATTTTTTTTATTTACTTCTTTTTGGTTAGAC
TAAAAATTCTTTATATAAAAACTAACTTTTACATCTCTATATATCATAAATATACTTAATCCGCTGAATT
TATTCACCTCTAAACTTTAATTATCATTTAATTTAAAAACTAAATGACATATTAGCTCCCTTGAGGATAT
TTTCTAACAAAATTATTAAAATATTACAATGTTATATGCTAAAAATTACAAAATTTTTGAATAACACATG
CAATATAATAATTTAAATTATTATGAGTTTAACAATTCATTTGCACTTTTATATAAATAAATAAAAAGAT
GTTAATAAAATAGTATTTTGAGTCAGGATATAGAAAATATTATAATATTTTTTTACTAATATCTCGAGTT
AGACCAAATTTCAATTCATAAATTCAAATAAACAAACGTAATGTTTTTATTTCAATAATACACTTGACGA
GAAATATAGATTGCAGGGATAAATCTAAGAGAAAATGAAAAAGACGGACCACATTAGCAAACTGCAAGGA
CTGCAGGTACCAAACACATTATCACTAACCGACATAAGTGAAAAAGAGTGGAATATCATTAAATGGACGA
TTATGCCCTTGGTTTTTTTATGTTGCCCTAGACCCTTCGAGATGAAAGGCCAAGATGTAACATAGGTCGG
AATCAACGGCTCACAATCCTCATTCAGCCCTTTATATTTTTCTCTACCAGCCACAAAGCACCTCCTCTCT
TTCTCGCTCTCCAGGGTTTCTAGTCCAGGGTTTCTCAATCCTGCGAAAGAAAGAAAGAAGGAAAGAAGCT
GTTTCAAAGTTCAAA
Sequence ID: 127
Sequence Length: 3630
Sequence Type: DNA
Organism: Poplar sp.
TCTAGACCTTAAACCCTCAATAAGGGTAATCTTTTCTAAAAGCAATACCATGTAAATAAAACACAAGAGT
TCTAAAGAAGAAAATGTTTGTTGGAAACTTAAATGCTTAAGAAATAAAGAAAAATAGTGAAGAAAGCAAT
AAAGAAAATAAAAATGATAAAATCCTTGGGAGAAAAATGATATATTATAGGAATCTAAATATACTACAAT
GACCATTATTTGTAGTAATATCTAGCTTTATTTATGCTATTACAATGTTTGAGTCTTGTTTACATAAATA
ATTTTAATTCTATAAAAAAATTGGTATAAGATAAAGTAGTTTATTAAATCCTAATGCAAGTCAAACTATT
TTTTTATTAACACACACATTAATCTTAATTTAATCTTTAACTTTGAATATTGGTTGAAACCGCATTTTCC
AAAAATTTAAATTTATTTTGCTAAAATTATTTATTTTTTTGTTGTTAGATTGTTTTGATGTGCTGATGTT
AAAAATAATTTTAAAAAATTAAAAAATATTATTTTAATACATTTCTAAATAAAAAGCACTTTGAACCGCA
ACCGCTACCACATTTGTGAACACACCCTCAATATTCTTTATGCTTGGCATATGTATATTTACTTTAAGAA
TAAACACTCAATACTGAAATTGAGAGGTCCTTTTATAGAGTAATTAAAACTGCCTATAATTTATTAAAAT
TAATTTAATTGGTCCCTGTAGATTTTAAATAATTAAATATTTCCTTAACCAACAATGAAGATGTCTTCGT
CTATAGTCAATCCGATTTTCTATTAAATACATGTTAGTTTTTTATTGGTTAGAAGAATAATAAAAAATTG
TTAAAATCTCAAAAACAAGTATTAAGAAATTTAACACATAAGTGTTAATTTTTCTTACCAACTTTTTAAT
TTATTTATAAATTTAAATGTTTTTGAATTTTTCCCTTTATTCTAAAGAGAAAACATAAATTAAAGAATGG
TTGAAAAACTTATTGTTAATTTTTTATTAATATATGTTGTATTTGTTATTATTTTTTATAATTTTTATTT
TTTTAGCTAACTATCTATTTATTTACCATAGAAAAGTGTCTTTTCTTTATTTAATTTATTTTATTAAATA
AGTGTTGCTTTATTTTTTTACTTCATTAATGATATTTTGTATTTGTTGTTATTTCTTAAAATTCTTTATT
TTTTAGCTAACTGTCTATTTATTTATCATAAAAAGTGTCTTTTATTATATTTTTATTTTTTTAAAAAAAA
ATAAGTAGTTATTTTTTACTTCATTAATGAAGGATAAAATTATGTATTTATTGATTAAACATATATGATT
CAATTTTGTGTATTTTTTTCATAGGCATGATCTTTCAATTTAATTTTGCATGAAAGTTTTGATTTCATGT
TTAAGGTTTTATAATCTTTACATAAAAAGTTTTTCTAGAAGTTTTCTTTTGATTTAAAAAATAAAAAAAA
ATTCAACTATAAAAAAAATAATCTAGCATAAATTAATAAATAATAATCATTTTCTTATCTTCTTGTATTT
TTAAAAGTATGTAAAAAAACTTCCACTATAATTAAAATTTTCTAAATATAATTTGTAGCTATAAATAATT
AAAATCTTACAAATTTTAACAATATTTTTTTGTTTTTTGAATTTTTTAATATTTTTTTTTCAAAGTTTTA
ATATATATATATATATATAGAGAGAGAGAGAGAGAGAGAGAGAGAATCAATGAAAAGCTGAATCGTGTCC
ATTCTCCCCTTTAGAAGTAAAAAATAATAGGAAATTAAGCTGATTATAGACAAAAGGATATTTTCTACAA
AATTGACTAAATCATACGGACTGAATTACCCAAACAAAAACTATGAAAAAGTCTTTATTAAAAAAATCAA
TACCACAGGAACTTCTAATATCATTAAATCAAAATTATTAGGTACATCAAGTGGTGTCCTCCATAAAATA
TAGAGAGAGCTTCTAATATCCCTCTGGTGTATATATATAGTTCCACCATAAAATTATTTATAGGTACCAC
TTGTACATTTTTATGTATAGTTTTAAAACCAGCCGGCGGGATCTAAGACCTGGCTGATCCAGGGCTGAAA
CTGAGCTGAATTTTAAAAAAAAATTAAGAAAAGTCAAAACTCGGGGTGACCCGGCTGACCCGATAGGTTG
ACTCGGTTATCTCGGTAAAAAACTTGGTTGCAATCCGTTAATTTTTGTTTTTTTAATAAAATTTGATTAA
CCCGAATGATTTAATAACCTAGTTAAAATAAAAAAATCCAGATCTTTGACGTTACCGAGTTAGATTTAAA
AATTCTGATTTTATGTGAGAAAAAAGAATGAAGTTATATATACACACACTTCAGAGAGAGTGTACAAACA
CAGCAATGCTCCTCTGTACTGTCCTGTGTATTTTCATCTCATGTCATTTACATATTAATTTTTTTTTTTG
TAAAATTTTTATATTTTTAAATTATTTTAATATGTTTATATTAAAAATATATTTTTAAAAATATATTATT
TTAATATATTTTTAAATAAAAAATATTTGAAAAAACGATGTTAAACAAGATAAGAAAGGGAGACCACATT
TTTGTCTGTGGATAGATTTAGTTTTTGTCCCTCATCTATGTTGGACTTCAACCCCTATTGTTTAAAGTCT
TTCCATGTTACCCTTCTAGTTTCAAGAAGTTCCCAACATCATCCCCAACTTACCTTTTGAATTTAGTTTT
TGAGAAAAATATTATGACATATACGAGGATTTAAATGAAAAATGAGAGGAATGTATGAGGAAGAAAATCA
AATTATGAAAAATTCATATTTTTTAGTTATTTAGTGGTAAATGGCAAAACAATTAAAGAAATACAAGGAT
AAAATTAAAAAATTTCGAACTATATGATTAGAAATAGAACAATGGTGAATATATAGGGCAAAAAAAGAAA
AGAAAAAATAGGATCAGGTGTTGGAGGCTATAATTAATTATAAAAAATAGTAAAGTTTTTACGTTTTACC
GGCAGTGAAAGTGAATTAGATAGGTAGGGACGGAATCGGATGAGTCGGTCTCTTCACGCACTCAATAAGC
CGAGCATCATCCATCCAATCCATTCCAATTTCTTTACAGAGAATTGAAGAAGAAGCTTGATTCTCTCTCT
TTTTTTTAAAAAAAAAAATATTGGATTCTGTTTCTTCTTCAATCCATCCTCAGCTCCCAAAGAAAAAAAC
AAGAAGAGGAAACCACCTCTCTATCTCTTCTTCTTACAGGTAACTAATTCATTTATATCTGCTGTTACTT
TCTTATTTGTTTATTTTTAGATCTGTATGTATATGCATAAACAAAATCAGAAGATATGAAGAAATCGTAA
ACCCTAACTGTTTTGCCGCTTGGTAGATTGATTGATGGGTTTTTGGTTTTATCGGGATTCGCTTTGTCTT
AAGATATACAGCACATGAATTGTAGGGAGAAAGCCCAAGATTTTATTAATTGAGTTATGGATCCTATGCT
GTGATAACTATTAGAAGGATTGTCTATTTATATCTTTCCAATCATTGTTATGTTGTGTTTAGTGATGGTT
GAAATTGAATTGGGATTTTTCTAAAGGTTTGATGATTTGGCTTTGCAGGTGCCCCCCGCA
Sequence ID: 128
Sequence Length: 3518
Sequence Type: DNA
Organism: Poplar sp.
TTAAAATTAAAGTAAGTTAAACAGTAGTGAAAAAATAATATCTTTTAAAATTATTTTAATGGAAGGTTTG
ACATAAATAGAAGACAAAATACATAGAACAGCAGTAATGCATGCTGCTAGAGCACACCTACAGACCTAAT
AATAAAAACACTAACACAAAAGCCTTATGATTCAACAGATAAAAATAGATAAGCATTTTCAAAAACATTT
AATACAAAAGTGAGGGAAAAATATACCTCTTCTAGCTGCTGCATCAACCTGCTTGTTGCTAAAAGTCTAA
GCACGTCAAATCCACCTTGGTGTACTTTTTAATTAAGTTTTTTTATTTTTTTATTTTTTTTCCTTTTATT
TTTTTTAAACTTCTTATAATTTTTTATTTTACCCTTCAAATCAAATTTATAATTTTTTTTTCCAGTAACA
ATAATTTTTTTTTAATTTATTAATAATTATATTAATAATATTAACAATAATAGTAATGGTGGTGGTGATA
ATAATAAGCATGTCTGACCCAAGTTTAAGTGGGTTTGACAAGCATGTCTGACCTATATTGGTTAGGTCTA
GCAAGCATGTCACGCTCATATTTTTGGGTTCGGTAAACATGTCAGACCCATATTAGTTGGGTCAAGTAAG
TATTTCAGACCCACAGTATTATTCAATTCAATTCTTTTGCTTTTATTTTTTTTATTTTTTTCCCTGATTT
TTTTTTAAAATTATTATAGTTTTTAATTTTACCATTTAAATCAAATTTTTGGTTTTTTTTCAATAGCAAT
AGTATTTGTTTTCAATTTTATAATAATATTTAACAAAAATAACAAAAATAACAATAACAATAATAATAAA
CAACATCAACACTAATAATTTTTATTTTTACCATTTAAATCAAATTTAAGGGTTTTTTTATAATAATAAT
ATTTTTTTCAAAATTATTAATAATTATATTAATAATAATGAATATAATAATACTTATAACATTAAGCATG
CCTGATCCAAGTTTAAGTGGTTTGGCAAGTATGTCGAATTCATGTTGGTTGGGCCTGACAAACATGCCAA
ATCCAACAGACCCGTATTAGTCGGGTCTGACAAGCATGTTAGATCTACTTTCTTTCCTCTTATTTTTTTT
CTCTCTATTCTTCTCAAAAATGATTTTTAAAAAATCTATTTAGAATTTCAAAGTTGTATTTGAACTATTT
CAACAAGAAAAGACAAAAGAATCCTCTAATTTAAAAATTAAAAATTTGTATCAAATCCAAACAAACTATT
TTATCCGATAGAGATTTTGCCCGAAGAGCAAATCAGAATCTTTCAATTTGAATGGTGATTGAGATTTTCT
GTTCTTAGAAATTATCTGCGTCTTTTGATATATCCTAATATTAATTCGATCTCAATATGAATTTATTATA
TTAATATGATTTCGTCCACAGTTTATAGTGGGACTTCCAAGGGATTTTTTTTATCAGAAGTGTGAAAGAT
TTTTTATGGTCTTGATGGAAAAAAAATAAGAAAAAAAATATTTTCATCGCTCACAGTACATAAAAATTTT
ATGTAAGATTTTTATGTTAGTTACAGTACCTAGCAGGAAAATAACTGAAAGTCTAATTTACTGAATTTTA
ACTGGAATTAAGAATATAAATATGAATTGGAATATGTTTTTTCTAATGTAAATAATTATTATAAAAGGGA
AAAATAAAATATTTTATAGACCCAAGATTCGATATTATTTTTATTACTATAATCATCATCAAGAATATAT
ATTAAAAATCAAGGATATGAATTTAATTAATTTTAATGTAATTTTTTTGTTTCTATTTTTTTTTATTATG
GGATTTCTTGTTGTATTCGATGCTTAAAATAGATTATCAATAGACATTAGATTTAATAATTTTATTTTAT
ATCAATTGTAATCTTAGTTGTTGTGTATCAAAACTGATATATTTTTTTATGAATTATATTAATATTTTAA
TTTCATTTCCAATTAGAGATAGATTTGAATTCATATCATTGACATATAAAAAAATAATATTTTTTATATA
GAGTAGTCTTTTAAATACCTACGTATAATTTAAATATAATGATATCTATTTAATTTTTAAGAATATCAGG
TATGGATTTTTTAAAATAAGATACAAAATTTGTTTTGTTTTGATATATATAGATAATACCGTGGGCTTAT
CCAGTGCAAACACAACAACGATTCACTCGTAAGCCCAATTGGCCGTAACGAGCAAACCCGTGCCATGGAA
TGACACCGAACAAAGATTCGTCAAGCCTTCTAATAAAAATCTCAGTTTTTTTTATTTCAATATTTAAAAT
TAGAAAACAAATTATCAATTTTTTCTCTACACCTTCAATGTATTCATAGACTAAAATAAAAACATGCTGA
CTAGCATAATACGTACTAGTGATAATTCTTCATTTTTTATTATTTTGGCTAGACTTATATTTATTCATTG
TTTTCAAGACGAGCTATTTACACCATGGTTCTTACAATCTAGAAAAATTAATTAATTAATTAATCTTTTC
GGTTCTATCAATTATATAATCTTTAAAACACACATAATTTAATTTTGTCTATACATTTTATGTGTTTTTA
TAATTTTTTTATTTATTTAAATTAAAAAAAAAAGCCAGGAGATGGGAAATATCAAACCATTGAAAATACA
TTTTTTCAAACAAAAGGCTATCAACCTTCATAGTTATTATTTTTATCACAAAGATTAATTTTGCTGTATT
TAATATAAAAATCACACGCGTAACGTGGAAGAAAAATAAAGGTCACAAAGCACGCGCGTATTTGAACCAA
TCCTTTCAATGCCGTCCATATCTATCCTACTAACCAGCGTGATTACCACGAATATCATATCTTGCCAGCT
CATATGTGCCGTCCATTTCCACACCATTTAAACGGAATCTCAGCCCTCCATTATTCAACTAAGCGACTAA
TCCTGTCCGTCCATTCTCCCAAACCCTCTATATAATAATCACATACACAACTTTCATATCTACCTAATCT
TATCAGCAAAGCTCTAGTTTCTCCATAGATCTACTCTTCATGGATCATCCTTCGATCTTTTCAATTGCAG
CTTCATATAGATCGCCTTGCGATCTTTATCTTGATATCCGTTTCGATGCTCGAACTCCATGATCTTGATT
TGCTCGGAGATAGCTCCAGAGGTATGTATAAGTGTTTTGTTTACGTTCCTAAGCTAGATCTGATTGAATA
TCTGTCTATTGGTTATTGATCAAGTCATTAATCCGCGTAAATCTAATTTTGTTCTGTAGATGTGGCGCGG
CTCTGTTTCGATAGCTCAAATATTAAGCGGATCTGTCCCTTCCGGCCATGCTTTCTTCGATCATGAGGAA
TTTTAGCAATATTTAAGTTTGGTTGTTGGCGGAAAGCAGAAGATGATTTGAGATTTTTTCTGCAGGTGGT
GTTGTAAGCAGTTACAGAATCCGCTGAACGTGTTGCTGGATGTAGACATGGCTGAAGATTGGTTATGTGA
GCTGTGGTGTAGCATGTT
Sequence ID: 129
Sequence Length: 3590
Sequence Type: DNA
Organism: Poplar sp.
TCCACTCTTGTTATGAGCGACGACCACAAAAATCAGAAATGTGAAGTAAAAATACAAAGAAAGTCACAAA
ATCACATGCGTACACCAATCAATCTAATTATCTAAATGTTGTTCTTGTGGGATTATTTTTTAAAATATTT
TGTTGATTATAAATATATTTTTTTATTTTTTAAAATTTATTTTTGATGTTGAATATTAAAATAATCAGGA
AAATATTAAAAAAATAATTTTAATTTTTTTAAAATTATAAATGAACCGTGTTTAGGCCGTCCACCTATTT
GAGATTGTGATAACAGTCACGGTTTAAAGTGTTTTTTATTTAAAAATACATTAAAATAATTTTTTTTTAT
TTTTTTAAATTATTTTCATGATCAACACATCAAAAAAAAATTTAAAAATATATAAATTTTTTTAACAAAA
ACAAAATTTTAAAAAACATGACAAAAGATCTCGTAACATTATTGACTAATTAACAATATCTAAGTGGTGT
GGGGGAATCACTGTTCCCCCACACCTAAATCACTGTGGATTACAACAGTAATTCACGATGTTTTTTCCTC
TTTTTTTTTGCATTTTTTCATTTTTCTTTCCAACTTTCCCTCCTTTTTTTTTCGTTTTTGTTTTTGAAAT
TACTTTCCTCTTATCTTTTGCTGGCTTGAGTGGCTGAGGGTTTGGACATATTTGTTCATACAAGTTTCAG
CCATCCATTATATACAAATTGTTGAAACTGACATGACATCTTTATTGTGCGTAGCAGAAGCTTTGGAGGC
TCTTGTTATAATTTTTAGTTGCCTTCTTTGACATAGGGATTTTTTTGGGACTGTGAACAAGTTGGAGGTG
CTGCAACTAGGAATTGCTTTCTATTTGTTTGCAACTGGTAGGCCTTGTAACATTCCCTGGAGAAGGAGTG
GATTGAATCTGAAATTAGCTCGCTCATGTATGGTGCTTGTAGTTCATTTTTTCCTTCCTTGTCAAATTCT
CATTAGCCTCCGGATGCTGTTTCATTCAATCTTTGTTCTTCTGTTGATGTATTTATATTATTGCTCTTTT
GGGCTATTGTGAAAAATATGGTCAGTAAAAAGCCTAGAAAAAACTCACCGGAATAATAATTCACGATGTT
TTTTCCTCTTTTTTTTTGCATTTTTTCATTTTTCTTTCCAACTTTCCCTCTTTTTTTTTTCGTTTTTGTT
TTCGAAATTACTTTCCTCTTTTTTTTTCCAGCTTTGCTTTTTTTTCTTTTTTTTTTCCATTATTTTTTTT
TAAAAAATTATCTTTGTTGATTTTACTTTTTAGATATTGAGCTGGTTAAAAATTCTGCTTTGTAATTTTT
TTCCTTTAAAATACTTTGGATTGCTACGGTGTTTTCCCACATAGTTTTTCTATTTTATTTTTTTTATTTT
TCAAAATTATATTTGTTGATTTTTTTTATATTGAGCTAATTGAGAATTTAGTTTTGTAATTTTTTTATTT
AAAACATTGTTTATTGCTACAGTGTTTTTCCGCGTGATTTTTTTTGTTGTTTTTGTTTTTTTATAATTTT
CTCCGAAATTATCTTTTTTTATTTTATTTTTTAATATTGAACTGATTAAAAATTACAGTTACAATATGCG
AGGAAAACACTGTAACTTTCCTCGCAAATTACTGTGGATTGCTATAGTGTTTTTTCTCATATGGTTTTTT
TCTGTTTTGTTATATTTTTTTCTAAAATTGTCTTTGTCAATTTTATTTTTTAAATATTAAGCTGGTTAAG
AATTGCAATTATAAGTAAATACAAGTTTTTCCTCACAAAACACTATGGATTGATACAGTTTTTCCTCACA
TGGTTTTCTTACAGTTTCTTTTGTGTTTTATTTTTTTGTAATATTTTTTTCCAAAATTATCTTCGTCGAT
TTTATTTTTTTTAATATTGAGTTGGTTAGAATTTAACTTTGTAATAAAACTTAATCATGTGGGGAAAGTA
TTGTAGCTTTCCTCACAAAACATTGTTGAAAATTACAATTACAAGTCATTACAAATAAGGTTAAATCATG
TGGGGAAGCACTGTAGCTTTCATCACAAAACACATGGAATTGCTACAGTGTTTCCAACATGATTTTTTCC
CCTTTTGTTGTGTTTTTTTTCCCTAAAATTATCTCTGTCGATTTGATTTTTTTAATATTGAGCTGATTAA
GAATTTAGCTTTATAATTTTTTTCTTTAAAACACTGTGAATTATTACAGTGTTTTCCCATATGATTTTTT
TTTCTCCAAAATTATCTTTGTCCATTTTTTTTTAATATTAAGTTGGTTAAGAATTATAATTACAATAAAG
CTAAATCATATGGGGAAAGCGTTGTAGTTTTTCTCACAAAACATTATGGATTGCTACAATATTTCTCTAA
ATGGTTTTTTATTTTATTTTATTGGGGAAAGCATTGTAGTTTTCCTCACAAAACATTGTCAATTGCTACA
ACGTTTTTTCTCATGGGTTTTTCCTTCCAAAATTATCTTTATTGTTTTTTTTTTATATTAAGTTGATAGA
TAATTTAGCTTTGTAATTTTTTTTGTTTTTTATTAACAGAAAAGCTAAATCATGTGGCAAAAACACTGTA
TCTTTCCTCCCAAAACACTGTGAATTGCTACAAATCATTTTGTTCAGTCTCTAAGTTTTGATCACCAACA
TAATTTTTTTTTTCATTATAAAATATTAGCTCCATTATACTTTTAATTTCTACTAGTGATAATTTATAAT
TATAATTATAATACTATTAAATATATTTATTTTATAAATTCGGGGCATGCATCTTGTCTATCTTAACCAG
CGTGATTATTACGAAAATGATGAAGTCAATATCTTGCCAGCTCGTCTATACCTTCATCGCCACCTACTTT
TTCAAACAAGGCAACCACTCAACCACGTAAACTAAATCTCAGCCCTCAATCATTCAACTAAACTACCAAT
CCTGGCCGTCTATTCTCCCAGATCCTCTATATAGTCACACACCCGCCCATCATTCTTTATCTGCCTTAGC
AGCTAAGCCCTAATATTCGTAGATCCACTCTCCACGGTACCTCATTCGATCTTTTTGATTCTAGCTTAGC
CTAGATCGCTTTGCGATCTATATATTGATACCAGATTTGATATTCGAAGCTCATCCGATGCTTCAACTCT
ATGATTTAACGGTCTGCTATCTGGTTTGATCGGAGATCGATGTACGTATCAGTATTTTTCTTATGTTTTC
CTTTGCTATAACTGATTTAAACTGTGCTTCCGAATTATTGATTAAGCTATTCATTTGATGTTGTTTTGTA
GATGCAGCGGAGATCTGTTTCTATGGGTCTAATTTTCAGGGGATATATCCCGTCGGTGTGCAAGCAGAAG
ATGACTCGAGATATCATTTGAAGGTGGAGCGATAAGCAGTTACAGAATCTCCGTTGCTTGATCATGTAAC
CTGAGCAGTGATTTGTTGATTGATTGAGGAGTGATGGAAGGAGATAAGGGGTGGGGCTGTGTGTTTTGTT
TATGAGGATGATGTATAAGATACATATATCGGGACCAAAAGGCCAGGAGGCTGAGAGGTCTTGTTTATTA
TCTGTCATCCGACCTTTGCC
Sequence ID: 130
Sequence Length: 4214
Sequence Type: DNA
Organism: Poplar sp.
ATTATGCAAATGACGCCATAAAGCCATTTAATCTTCGATAAGTTAGTGTATGATCTTTATCCCTGTAAAG
AAAGTTTGTTTTGCCACAAACAAACAAAAGAAAAACTCACGTTTATTCTCCAAATCGCTGCTGAAATTTG
TAGGTAAAAAAACATAATATAATCTCCTCTAACGAACCCAAATAGGCTCCTTTTGGGTTGCAAGCTAATG
AGCCTAAACTAGTAATCAATTAACATAAGTCCAATAATGAAATAAATCCCTAAACAATATCTAATGAGCC
AGAACAAACCCAAATTAAAAATAATTATCCAAAATTAATTAAATAAATAAAATAAGATAATAAAAAACAA
ATTCTTCATGCTAAAAGTCCTCCATGTAGCCCGAATCTCTATTTTTCGCACATTCTTGGCAGATTTTCAG
TCTTGACTTTAAACACGTCGTTCCTTTTCTTTTAGATGAATTACTAGGCCTACCATATATGGTTAGAAAT
TTTGAGATGTCTAGTTTCCAGCCCAACTTGAATCGCAGAACAATTACACATGTAGCTCCAGATATGTCCT
AAATAGTACACAAAGGTCTAGTCTGACAGATTTGACAAGATTTGTCTAGTAACTTTAACCTTCTGGAATA
ATATGTTCTCGAACTTCATTTAATCTGAAAATTTACTAAAATATATTTCCATGTCTTTAGTAGCTTCCTG
TAAAATTTCAGCTCATTTTGATATATAGTTTGAAAGATATGTTCAATCTTGCAAAATTGGTCAGCAAGCA
TTTTAAAACCAAATTCTAACCTCACCTAGTTTGTATCAACTGTCTTTCCAAAGATTGTGTACATGTCAAG
GCTAATTTAGATAAAGCAGTTATTACAAATTCAGGAAAGTGAATTCGGGAATTTCACAAATGCTCTACTT
TATTGTACATGCTTTATTTGATTTAAAAAATTCATAGATGTTAAATAATCTAATTTTGTTAAAGCAAGAT
TTAGGAAGGGTTTCAAGTAATATATGTTTGATCTGATGTAGCTGATTCCTCCAAGACTAAAACTTATGGA
GAATTATGTATTTCCTTGTTCATTTTTACAAAAAAAGAAGTAACAAGCTATGGTGATAGTCACAGTTTTC
AATATGAATGAGAGAGGAAAGAAAAGAATAGGGTGATAACATATGTATTTAGTTAATTTTCACAAGGCAA
AAGCTCATGAAGAGCTATGTATTTCCTTGTCCATTTTGACAAAGAAGGACGTAACAAATGATGGTGGTAG
TGATTGGGCTTTATAGTGGAAGGCATAGTTTTGAAATCTAGCCCGGCTGGCCAACCCGGGACCCGGCTGG
TCCGGGGCTGAAATCGGGTCGGGTTTAAGAAAAAATAGAGGAAGTCATAATCCGGTGTGACCCGGTTGAC
CCGACAAAACCCGGTAAAAAATCCGGTTGTAACCCGTTGACTATTTTTTTGTTTTTTTTATCGAAACGAT
GTTGTTTTAATTTATAAAAAAATAGGGTTGACCCGGGTGACCCAGACAAAACCAGTGACCTGGGCCTTGG
ACCAGGTGGACAACCAGGCCGGGTTTCAAAACTATGAAGGGAGGGGCTACCATCATGTTTCAGAGAGAGA
GAGAGAGAGAGAGAGAGAGAGAGGAAAAACAAGGAAAATAGTGGTAGCTTTGTGAAGAATTTTTCTAAAG
ATACTTTTGATATTAAAAAACATCACTATAGCAAGCATATAATATTTTATATGTTCTCTTGATTGAGTTG
TAATATATAATGTGAAATTAATTTTAAAACCACGGATATGCAAGTGTGATTTATAAAAAAAAAATTGGTA
TGTAAAATATTCATGTATTAAAGAACATGAGCTTTTTAACAATTTTCTCAAATATAAATATTATATATAT
AATAAAAATTTGGATTAAATTTGATAATATTTATAGTTTATATATTGTCCATTGAATAACATGACTGGAA
AAAAAAACTCAAATAAATTGATTAAAGTCAATACAAATGGATATTTGAGTTTGAAATTTGCGGTACCAAC
TCCAGGTAAACCATTCACATCATTGTCAATTACCTAAATCCGAAGAGCCAAAAACTTTTTCCCAAGCCGC
CGAAAGAGACCCAAGCCCATGACTCAAGCCACCGAGCCGGCTACCGATTGAGGTTGGTGAGTAACCAGCC
ATGCCGGTTATTTCAATCAACCAAATTATTATTAGTAATGGTAACTGCTTTGTTTTTTCGTCTTTTTATC
TTTTTCTTCTTAAGTCATTATTGTTTAAAAGTAAAATTTAAAAAAGAAATAATTACAAAAATCTTTCGAG
ATCTAAATTTTATTTTTCCACTGTATTTTTATAAATTATACTTTACACCCCAAATTAAATAAAAAATTCA
AATAAAATATATTATTAAAAATACAAACTATTATTAAACATATAAACTATAGGGTGAAAGGTGTAATTTA
TAAAAGTATATAGAAAAAAATAAAATTTAAATCATACTGTAAATTTTTTTTTTAATTATCCCATTAAAAA
ATAACAAAAATAAAATAAATTAAAAATACATGAATATTAAATTAACTGGATCTATAATTACACTTTATTT
TAAAGTTATTATAGGATTATTTAAGTGGATTCTTAAAATATTTTCAAATTATATATTAACAAAATTAACC
CATGAGCTAGAAAGTTAATATCAACCCTCGAAGACCATATTAAATTTGAAAAGAAAAATTTAATCGAACA
AAAATAGCAATACATCTATATAGTTTTCCAAGAGTAACAAAGAAAAATTACTTCCAAAAACATTATTAAA
CACCTTGAAAAATGAAATTATTCTCTCATTTTTATCCATCTTCCACCAACCCCAACCAATCCAAATTCTG
ACATCCAACGGCTATGTTTTGTCCCTCAAGCAATTGACAGAATCCACCGTAGATTTAAGAGAATCCCAAA
CCACCCCATCCGGTTACCAGTAGGTAGGTCTATAAATACTAAGTAACCAAAGCAGTTTTATTTCGTGATT
CTTTGGGGTCTGATTAGAAACTCTCACACAGGCCGACAAACTCTTAAGCTTTCCTTTAGATCTGTACCGG
TGAGTCCTGTCCAAGGTACCTCCTGCCCTTCTCCACTTTATAGTACTGTTAACTCTATATCATATTGCAT
GCTCTCTTCATGGGTTTTCTTTGCAGACTCTAGAAAAATGAATGTTTAAAGAGTGGTTTTTTAGTTTCTT
GGTCTTAAGTTTTTTAGTAAACATGGTTCAAACAGTTACAGATCTGGGTACTTGTTGCGTTTATTCTGGA
GTCTGGTCTGTGTCGGGTGTGACGTTGAAGATTTCGTTTTGACTGTTAGTGTGTGCAGAACATCATGTCT
GAAAGGCATGTGTTTCGCTTACATTTTCATTAAAATACATCAGATCTGATTATTTTTATCAATTTGATAG
TTTTTTAGTGATGAACTCTCTGAAAATATATCGACAGATTCCAAGAATGTTTTGTATTACTTGAAAGGAC
CATGTAGGGTTGCGATCATAATCGTATCAGAGTGCCTCTTGCTTAGATCTAGTGTTTATTCATTTCTATC
GTATAATTACTAATAAATTATACCAAGAAAAATGTAACTTTGCGCTTCTGCAAGTAACTGAGGCACGTTT
GGTGTCTCTACTTTTAATATAATCAATGGTAGAAAAAAAACTGTTATAAACTGTGCAAAACACCTTCAAT
TCATAATGGGGCTCACGATACTCTGGGAGCTCACTCTGTATGAGAAGCTAATATTTTTGTTTGCCGTTTT
GGTACTTTCACCATATCATAGCATGTTAATACCTGAATTTTCACTATTGGGGGTTGTTTTATTGGGTATT
TGATTCTTTTCCCGACTTTTTTTTTGACAAGTGATTCTATAGAAGTTTATCTCTTCCAGTGCTAAAGGAA
TCTAACTGTTCTTTGTTCTTTTTTAATTGCTCTCATTCAGGAGCATAAACTTTTGAGTTTCTTATCATTT
CCTGTACTTTGATTCTGCCCGTTTATAACTTGTTTTAGAAAAGTTTGAAGAATCTTAATCAGTCATGAGA
ATATTTTCGGCACTATTCTTTTTACCATATTCTCACATGTTGATGGAATTTTGCTTGCCACACTAATTAA
GCAAGCTTAGTTTGCACACAGTTGCCACATGTCATCATATTAAGAGATCTTTGTTGTGAATTTTGTGACA
TGCAGCTCAAGAAA
Sequence ID: 131
Sequence Length: 3000
Sequence Type: DNA
Organism: Poplar sp.
AAAATATTATTTTAATATATTTTATAATAAAAATCACTTTAAAAAACAACCACTATCTTATTTTCAAACA
AAATCATAATAATATCAAAGTTGGTGACGCTTACCAACCCCCGACCGATTCTAACTTTTTTTCTTAATAT
TATTTTAAGCTTATTTTGAAAATTGAGTCGTTGTAGGTGTCAATTATCATATTGTTCTTGTTGAGTGCAC
ACATGTTATTTATATATAGTATTTGTACGGTTGGCACGTGAACTTTCCTAATTTATGTGCATCATTTATT
GGGATACATAGGAATTCTCATTGATCCAATGAGGGAATGGAAAAATAAATAAATAAAGAAAACTAAATTA
TAATAAATAAATTTAATTTTTAAAAAAAAACCTCCTTACAACCACCAAAATAAACGGGCAATGTCTTTCG
ATCATAGTGGTAAGAGTTTGGCATCAAGATATTTGCTATTTCTGTGGTCTCAGGTTCGAGCTCTGTGGTT
GCTAATATGATGGCCACTGGAAGCTTACATGGTCGTTAACTTCAAGACCCGTGGGATTAGTTGAGGTGCG
CGCAAGCTGATCCGGATACCCACATTAAACTAATTAAAAAAAAAACTTCGCTGATGAACGAGGACCACCA
GCAATAAACCTCCATGCTGATGCAGTAACGCGTCATGCTGCGCGTGGGATTAACCATTCACATCGTTATC
AGTTACCTGAATCTAAAGAGCCAAGCTCATGACTCAAGGTCACTGAGCCGGCTACCGGCTAAGGTTGGTG
AGTAACTAGTCATTCCGGTTATTTCAATCAATAAACAAAACAAATCATTAGCAGTAACCAGCCAAGGGTG
TTTTTTTTTTTTTCATTTTCAAAGACAGTATTTCTTAAAGAATAATGTTAAATATTAAAAAACAATAAAA
GAAATTTAAAAATACACTCGACCCTTGAATTAGTGAAATTCATAATTGAATATCCCCTTAGAATCACGAT
CAAATAACCTATTATATTCTTTGATTTTTTTATTTAATATTTAGTAAATTAAATTGTATGATATATAATT
AGATTCTACCAAACTATCAGTTTTTATGAAATTCAGTCCATAATCTAAAATGTTTAAGATTTCATATTGA
ATCCGGAACGAAAAATCTGGTAAAAAAAAGCATCATATAGTTTTTCAAATGTAATTAAGGAAAACTGCCA
CAGTTTTCAACACCTTGAAAAATGAATTTAATTTCTCACTCTACGTGGGAGTGTTTCAGTAAATTATCAT
ATTTTTAAAAAAATAATACAGAAAAAGAAATTAAGAAATTGTAAATTATATTTATGACACTTGTTTCACT
AATAGAGATGATGATAACAATCTATTAAACTTTTTGTTAAAAATATAATTCACACAATCATAAATAATCA
ATTCTCCACTATTAAAATACAATTCACATAAATAACACATAATATATTTAATTATAAATAATAAATATAC
ACTAATAATCAAAACAACATAGCATCTTATTAAAAATAAAACTAACTATAATATCCAAACTAGTTTACTA
ATTATATTTTAAAATATATTACTTTTTTTTTTTATGACGATGAAAAACAAAAAAAAATACCCAACCTATC
TCCCACCAACCAACCCAGCCCCCAAATTCTGACATCCAACGGCTATGTTTTGCCCTTTAAGTAATTAGCA
CCACAAGATACAGATTCCTCCGTAGATTTAAAAGAATCCAAACCACCTAATCCGGTAACCAGTTGGTAGG
GTAGTTGGTCTATAAATACAGAGTAACTAAAGCCGTTTTATTGTACCACACCACCACCGCCTAAGAAATC
TTGAGGTCTGGTTAGGAACACAAACACAGGCCGACAAAGTCTTGAGGTTTTCTTTAGATCTGTACGGGTT
ACTCTAGTCTAAGGTATTAACTCGTTGCACTTTCTCATCTATATTTAGCCTTCATGCTCTCTCTTTATGG
CTTTTGTTTATCAACTCTAGAAAAATGAATGTTAATGTTGGTTTTCAAATTCAATATAGTGGTCTTTAGT
TTTTGAGCTAACTTGGTTCAGACAGTTTCAGATCTGGGTACTTGCTGGTCTTTTAATCTATTAGTTCAAT
CAGTTTGGTTTACTCTGCTCTCTTCTTGTCGCGTGACTTTAAAGATTTCGTTTTGAATGTCAGTGGGTGC
GGTACATGATGTCTGGAAGATTAAACTTGTCATGTGTTTCACTTACAACATTGCAGTAGAGTGCAGAAGA
TCTGGTTTTTATTTACAATTTGATAGTTTTCTTAGGGGTAAAAGTGTTTAGAAATATATCCAGCAGAGTC
GAAAAATGTTTTTATATAATTGGAATGGACCATTTATGGTCGCAATCATAATCGTCTCAGTGTGAGTAGC
TTAGATCTGGTGTTTCTTATCCATTTCTATCGTACAATTGGATCAACAAGTTATGCCAAGAAAAAGCTAA
CTTTGCACTTCTGCAAGTAATTGAGGCACGTTTGGTGTCTCTACTTTTGATTAATGGTAGAAAAGAAACT
GTTGTAAACTGTGCGAAGCACCCCTGAATTTCACTCTGTGCGTGAAGCTAATATTTTTGTTTGCCGTTCG
GTACTTTCACTATAGCTTGTTAATACCTGAGTTTTACTGTCGGTAGATCTGTTGTTGGGTTGTCGATTTA
TTTTAACTTCTGTTTTTGACAAGCGTGATTAATTCTATAGGAGTTTATCTCATTCCAGTGCTAAAGGAAT
CTATTTCTTCTTTGTTCCTTTTTTATTGCTCTCTTTCAAGAGTACAGACTTTTGAGATTGTCATTTCTTG
TACTTGGATTATGCCCTTATTAACTTGTTTTGTAAAGTACGAAGAATCTAAATCAATCTTGAGGATATTT
TTGGCTCTTTCTTCCCCATATTCCTACGCGTAGATGGAATGTTGCCTGCCATAGTAATTAAGCACACAGT
TGCCACATGTCACCATATTAAGAGGTCTTTTGTGAATTTTGTGATCTGCAGCTTGAGAAA
Sequence ID: 132
Sequence Length: 3238
Sequence Type: DNA
Organism: Poplar sp.
AAATGATAAAAAATAAAATACCGTTTTACCCTAAAAGGCAGCCAAGTGATAGTTTTATCAAAGGCAAGAC
CGTCAATTTATTATTACAATTCACAATAAACAAAGGAATATGTACAATGATTTTCCCACATGTTTTAACA
TTTTTATCTAATCTTTAATAAAAACATCTATTAATAAAAAAAATTAGAAATGAGGTCAGGTCATGCTTTT
GGCCCCCCCAAAAAAAAGGGCTCGTGCATTGACTTTTAATTGTTTTTAAATGAATAGGGCGAATGACGTA
ATGCTCTGGAAAAAAAAAAACAACGCGTCATCTATCAGTCACCAGATTCCAACCATTGGCGCGACTAGAA
AACCAGTGTTAGTTTTTTTTACCAAAAAATATATCTTCAACCCATTAAACCAATTAATGACCACAAAACA
CACAAAAAAAACAACTCAAACCTAAAATCAACTCGAAAATAAAAATCTCTTGGAGCTCAGATATGTTTGT
TTTCAGGTGTTTTTAAGATAAAAAAACACTTAATATTAACTAACCTATTAGAAAATACATAGATCCATTT
TAAAATATTATCTATGAAGACATATCCTCTACCCATCTCTATTTTAAAAATGCTGAAATCCATTTTAAAA
TTTTTTAAAAACAAATTTATTTTTATATCATTATTTTTATTTTTTTAATCAAATATATCAATCTCTCCAT
AATCCCTGTCCCTCTGGTCCCATGAAAATTACCGAACACTACATGAAATAAGGCATGTTTGACGCATCAT
TTCCAGGAGCATTGATGTAATTAAACGTTGCGTGTATCACATAAGAGAAAATGTTGACAGCCCTTTCATT
GTTTCAGGCTCTGCACCCCGCATTTTATTTCCAGCACTCCAAACAAAGCCAATTTGCACCCTTGAATGTT
TCAGTACACTCGCTGAAGTTATTTCTTGGGGATTTTCGAACGATTCTTCGAGTGGTCTTTTTTCAAATCT
TTTCACGCAGCTCCTTCACCGTATTGCTTCCCGGACCTTGTTGCCGGATAAGTGCATCACTGAAGGCATC
ATCCATGTGTTCTGTCCATTTAAACGCATCTCGCCTTCCCACATGATTCTTCTCTGATTTATTACCCGTT
GCAGATGAACAGAGGCATCAGACAAATAACAAAAGCAGGCTACCGACCATTAACGACCAAAAAGTACAGA
TGCTGTCACCTTTATGAAGATTTCATAAATTGATTTTAAAATCATCGAGTTGTTTAGCAATTCACTCTTT
ATTATTTATTTTTTTATTACCTTTTTCGTTGTTTGTTTTTTCTTTTGATTTTGTCCCTCACGGTTTTTTT
TTAATTATCTTTTTTATGTTTTTTTTTCTTTTGATTTTGTCTCTCACGGTATTATTTCAATTATTTTATT
GCTTTTTAGAGATTGATTCTTCGTTCTTTTAATTATTTTTTTTGTACTTTTTAATTTTTTTCTTTCAATT
ATTTCTTTAATATTTTATTTCATTTGTTTTTTTATTCAATTTTGATCTTCATTATTTTAATTATTTTTTT
CATCATTTTTTTTATTTCAATATATCCCTTAACATTTTATTTTATTTTATTTTAATTAAATTTTAATCCT
TATTTTTTAATTATTTATTATTATTATTTTTTAATTTGTTTTTTCTTTCAATTAGTCCCTCAACATTTTA
TTTTATTTTTTTTGTAATTCAATTTAATTCTTATTCTTTCAATTTTTTTCTTATCATTTTCTTAATTTGT
TTTTCTCTTTCAATTTTCCCCTCATATTTTATTTTATTTATTTTTTATATTCAATTTTGATCCTCTTTTT
TTTAATTGTTTTTTGTAATTATTTTTTTATTGTTCACTAAAGTGCCTTGGTATTTATTTCATTGGTTTTA
TACTAGCTCAAGTCCTCATTGTTTTGATTACCATTTATTTTCTTTTTAATTTTTTGATAGTTAAGAATTT
TAGACAATACTTTTTTGTCTTATATAGGGTCATATCGGTGTTATAATCAAAGTTACTAATTTTAAAAATT
ATTCCAATTTAACTTCAATTTTTTTTCAATTCTATCATACATTATGGTTGACAAATAATGTATTGGAATC
AATTCCCATTGCATCTCTTCTTGCCAATCCAATTGTGAGTGTTATTGGGCATTCAACTTTTTTTTTTTTC
ATTTTCTTTCTTCGAGGTTATCCCAGGTTAACTTAACTTTACTCTTTTTTTACTTAATCCTTCGATGTTG
GGCTACTGGGTTTTGAGATTTTTTATTTTTTGTATATTTTTTTAATTTCTATAGAGATATCACTAGTCAT
ATTTTTGTCAGATTAACTCAGTATGACTTGGTTTATTTATTTAGCTCCGATCCTTTTCTCTAGTTTTTTT
CTTAAAATAATTTTCTTATCACAATCTTGCATCACGGTTGATGTGCTAGTAGAGTGGACCCGAGTCGGCT
CAAGTGTTTTTATTTTACATTTCTTTTTTTAAATAGATTTGTTTGTAATTTTATCATTTGAAATTATATT
TTTAGAGTCCCGAGCTTTCTATTTTTTCCCCTTTCATTCCTAAGATGTTATCCTAGATTGTGAGCTAGTC
AAGTTAACTCGGGTTTATTTAAGTTATATTTATTTTAATATCTTCTCTTATATTTTAAAAAAATTGACCC
TGGAAAAAAAAATAGTAACAATAATAAGAAAATGCTAAAGTTGGGCAATGGGCCAGATTATTGCAGAAAT
CAAAGTTGATCCATGGGCTGGACCATTTCAGCAATCAAAGTTGGGCTTGAGATCCCTGGAATCCAACCAA
TCTTACATTATCATAAATGATTACTTATGTGAGAGCAAAGAAGCACAAAGAGAAGCACAAAGAGGAAAGC
CAAATATCAAAGGGTAAGGCGTCCAAAACTTAAGACCCACGCAACCAATAGATTATAAGCGCAAAGATCG
ATGACGTGTCTAAATTTCCAAATCCCGCCTAAACCCCTGCCACCTCCCCAATTTCATTTCACCCGCAATT
GAATTTCAATTTCCTTCACGACACTTAATATCTCCGCCTCTCCCTCAACTCTCTTCCTATTCTCACACAC
AATATCCTCTCTCTACCACTCTTCCTCTCTCTTTCAAACCCTAACCCCCCACACCGGTAAGTCTCGCAGA
TTCTGAACGGATTCTCACCCTTTGATCGGTTCTCATTGATTTAAATCGTGTTTTGAGCTGATCCGGTGTT
AAAACTGCAGGTTTCTCC
Sequence ID: 133
Sequence Length: 3013
Sequence Type: DNA
Organism: Poplar sp.
ATATATATATATGAATTTCTTCGTGAATCTATTTAAAGTAGCCTGTTTTGCAAAATTATTTTACATAGTA
GTTAATGAATTAATTCAATATTCGAGTAATGAGATAGATGAATTAATTAGATTTGATTTAGAATAATTAG
TGTGGTTTTAAATTTTTTTAAAAAAATTAAAATAATATTATTTTAAATATAGGATTAACTTGTTATTATA
AACAATCAAGTTTAACCGAATAAATTTTTATAAATTATAGGATTAACTTGTTATTTTCTATTATAATGAT
AATATATAAATAGAATAAATTTTACTTTTTCATAATAAACCTTTTAATCTCGGTGCATGAATAACTTATT
TCAATCAAATTAAGAACATAAGCTTAATTTCAATATGAAAACTTCAATGAAAATATTACCATCAAAAGAT
TGGATTAAAAAAAAAACCATAACACCAAAATTGTTTCAATATTATTTTTTCCTAGTGGGACCATCCAAGA
GAGGTCATTTGATAACAATGATCATAGTTGGTGATTGAAGGCCCCATTTAAGGCACCGGCCTAGACACCA
TTGTTGGCTTTCGCTTGTCTCCACGCCCGTCCTTACATTGTGAAATTGTTTTATGGTTTAAAGAAAGTGA
AAAGCTAAAAATAAAAAAAATACCTTAAAAATAGCAAATTATTTGACCACAACAGTGAAAAGTAGTCCTT
CATTATTGTTTACATACACAAAAGGTGCCATGAATTAGCCTTTCTTTAGCCTATTACTATGATTATATGT
GCAGGTAGGAGGTACCCAAGCTCCCGTACCATTTTTATTTTATTTTATTTTCGATATTGGTTCAAAAAGA
AGTTGGGCACCATTAGTTTCTGTATTTAGAAAATTAATTAGAATACAAATTACATGGAATTTTTTTTTTA
TCTATATAGTGAATTTTATTTTTTTTTATTTTATTGGTGAAAATATTTGAAGTTGATTTTGGTATATCAA
GATTAAATTCTATCGAATTTTGAACAAGTTAATTATTCATATTTAAATCAATATATACACATCTAAATTT
TTTTTGATATGTTTAAAAATATTAATAAAAAAAATCATATGTGCAAAAAATTTAAAGCAAGCAAACAAAC
AAACAAAAGGGTAAAACTTGTCTCTTGATTGTACTTAATTAGAATCTTTGAAAATAAACAAAACAATAAT
GTACAAGATTTGCATTCAACTTGCTTGGGTGGCTCTTGATGTTCTCATATGGCAAAATCATTTTGTACAA
ACAAATATATTGCTTGGCATGTGATTGTGTCAAAACATATTAAATTGTGTTTGTCATGACATGTACGCCA
CTCCAGGTTTTGATGATAATGAAGACTAGCAGTTCCAAGAAAAAAGCCTTAATTCATGAAACACCATTTG
TCTTTTAGGTTTTGCAAAAGCTAAATCATATACCATTTCTTTTAGTATATAATCTTACTAATTATAGGGT
TAATTAGAATGGTGAGTAGCACGCGAGCTTATCAATTTTTTTTAATGCACAATTAATATATATAAATGTT
TATGCAAGTCATTAACTTGAGAATATTCAATAACATCAAATAATATAATAATCTGACTTAAACTATAGAA
AACAATAGTAGATAGTAGATGAATCGTACAAAAAATATTTGATAGCAATTAACTAAAACAAATAAAATGC
TAATATCATACCTAAAAGGAGAATAAAAAAAATCGCTAGAAACTCACCGTTATTCTACTGTGAATAATGC
CTACCACCAAAAAACCTTGCTGAGACGGTCCAAATCTCACTGCAAAAGAGTGCATGACATTAAAAAAAAC
CTGCATGGATCGATAGAGCAGCATTGCAGGAAAAAAGAGAGGAAGGATACTCAACCAATCAATCTTATTT
AACTCAAGAATAAAATTTATCTAAATATAAATTTAATAAAAAATATTATTTATATCCACAATGAATTGTG
TCCAGGTGAACAATGTTTTCTCCTTCTTTTTCAGTTTTTTTTTTTAAAATTGTTTTTCCTATATGGCTTG
GTTGCATCTTGCACCCGATTTTCATTATAAAAAAAAATATAATGAGGTGAATTTGATTTTAATATACTTT
TTATTTTTTTGTTTGATGGTAAATATATTTTTAGACTAACTTATATGTTAACTGAAACTAAGTCTACTAT
TGAACATTTTAAGTATATTATCGAACATTTTAAGAATATTTCTCTTATATCAATCTGGTGTGTGAAAGCG
TGTTTGGCATTGCGACAACGGTTGATTTCAAAGTGATTTTTGCTTGTAAATGCATTAAAATAATATTTTA
TTATCATTATTATTTTTGACACCAACATATTAAAACGATCTCGAAACATTAAAAAAATAATTTAAAAAAA
TTTAAATTAAAAAAAAACTGCATTTAAAACACTAAAACAAATTAAACATATTCAAAATAGAAACAACTCT
TCTCAACTAAGAAAAGAACAAAATGAATTCTCTTCAAAACAAATATCAGCATGATCAACACGGTTAATTC
GATCCAAAAAGAGGAACCATTCTATGAAAAAAAAAAGAAAAGAAAAGGGGCAAGGTTAGGCTAGTACAAA
TTAACAGAAAATCAAATAGAAATCCTATACAAAACATGAATGCAAATTACATGATCTAGGCAGTAATTAA
CCTCCATTATTATGCCCTCTTAGAAAATAAGAACACATGGGAACCCACTCAAAAAGAAAGGTATGGTGCT
GATATATGAAATGGCCTCCACCATTTGGATTGCCAATTCATATTTGATTTTAAGTATTTGTCAATTCCAT
CAATTAAAATCATATAAAAATTATAATCTCAATCATACTTTATATGAAAAAAGAAATAACATATTTACAA
CTAGCAAGACATGCTAAGGTGTCTTGTCAGCAATATGAGGTGTCAAGTTGAAAGATTCTCCAAGTGAGCA
TGCTCCCTATGATCTCATGCTCTCATTCTTTAAATAATCTATGCATTTCACTCCTCTATAAATAGCTCTC
GCA
Sequence ID: 134
Sequence Length: 3287
Sequence Type: DNA
Organism: Poplar sp.
TTAAGATATTGTTTTATAAAAAACCATTTAATTTTTATTTTATAAAACATCAATAAACATTCTTTTTGTG
CAACCAAACATGGTTCTTTTATATTTTAGATTTTACTAGGTTGCTTTCATGCGAGCATCTATTCTTTTTA
TCATATAATTAGATAAAAAAATAATTAAAAAAAACAAAGTTATCAAGCCTAATTAGGTCAATGATCTAGG
TCGCGAATTAATCGAGATTAGCCTAAGTCGATTCAATATGTTGTCACCTCAACATTTGAAGAAAAAAAAT
TATTCTTGAATTTTTTTAAGATACTATGTTTTTTTACCGGTTATCCAAGTTATTTTTGAACTATCAAATC
AACTAGTCATATCAAAACTAATATGTATTAATATGTGTTTATTTACTTGCTCCGCGACTTAACATTTAAT
TAGTGTTAATATACTTGATCTATTTTATTTAATATATACATCAAGTTTTGAGTTTATATTTTTTTTTTAC
TAAAAACACATTAGTAATATATGCATATTATTTGTTAAGTAAAAAAAATTGCTCCTCGCGAGCCAGTTAA
CTCGTTTGATAATTTTATGTTTCAATTTACTAGTTTGATAACTATTTGTATCATAATGCTAATATGATGA
CGAATTTAATGCATCAGGTAGTTAGTTTATTTAAATAAACGACTTTAAAAAAAATGTATATTTTCATATA
TACATTCATATCAAATCACAAAAAACCATGATTGATAAGTAGCTTTTTAGGTAGCGGTACAATCTGTGTT
TTATATATATATTTTATTTTTGTTTAAAATTATTTTTTTATTTTTTTAGTTTATTTTAATATGTTAATGT
AAAAAAATTTAAAAAAATATTATTTTAATATATTTTAAATAAAAAAATATTTTTAAAAATAATTATTACC
ACAATTTTAAATATATATTTTATTAAAAGTGAGGGAAGAGTTCGTGCCACAAAATCACAAACTTTGGCCT
TATGGAATTTACCTTGGGATTTTTGTTTTGAGATCAAGGAATTGTTTTTAAATTATGCTACTTATTTTAT
TCAATTTACAGTGCATCTAAATACAAAGAAGTATACTTAATCCATGTAAATAATGCAATGGTAGCACATT
TATACACACTTATACTCCTGTTAAAATTATATTTGATTATTATTTCAAGATAAAAAAATAAAATAATAAA
AATAAAATGCACATATCTTCATATACTTTATATTTTAAAAATATAAAATAAAAATAACATGTTTTTATGT
ACTTTGTGTTTTAAAAGTATAAAAATATACGGTATATTTTGTCATATACTCCGTGTTAAAGTGATGTTTG
ATTATTGTTTTGAAACAAACAAAATAAAAATAATTTTTTATATCTTCTTAGAAATACCAACTAGACACAA
TGTAATGAATTAGAGTACAAAGACAATAATCAGTACATATCATATAACTCACCTTCCATTCAAGAGGGGG
GAAAACAACAACGTTGCTGTTGAGACAAGAATAACATTAAAATGTTAGAAAGATACTTAAAATATTGATT
GTCAAATATATATGATAGCAGGAACATGAAAATGGAATTCAGTGACTAAGAACAACATTAAAAAGGTGTT
CGAGAATGTGATAACGATTGTTTTTAAAAATATTTTTTGTTTGAAAATACATCAAAATAATATATTTTTT
TATTTTTTAAAAATTATTTTTAATATCAATATATCAAAACGATCTAAAAATACAAAAAAAATTAATATAA
TATAATTTTTTATAAAATAATTTTAAAACAACAAAACAAAAGGCCTCTAAAACACTACTATATGAAAGTA
TGGTTGCCTATAAGGGTGAGCAAAAAAACTGAAAAATCAATTAAACCAAGAAAACTGGAAAAAAAATAAC
CGAAAAAACCGAACCGTGAAAAAAAAACCGATTAAAATTTTGAAAAAACCGACCGGTTCAGTTTTATAAG
CCTGAAATAAAAAAAAAACCAAACCGAACCCAAACCGAAAAAAACCCGGAAAAAACAGAGCCAGGCCGAA
AAAACCCGAGTCAAACCGGAATAAACCGAGCCAAACCGGTTTGAACATGTTTTTCTTCTAAAAAAATCAA
ACCGTAATCAGTCGATTTGAACCAATTTCTAATTTTTTTTAAATTCAATTTAATTATTTTTTTTTAATAC
AAACCGAATCGAAGCGAAAATAATCAAATCCTGGTGGCCTAAAAGAGCTTGGGTAAAAAGAACCTCTTTG
GATTGTGGTACTCGGTTAGCCATCATAAATTTAGATGGGCTGAATATCTAAAAGACAAAAAGCATAGAAA
TTAAAAATGAGAATGAATAGCATGTTGCTTATAACTTTGGCATAGAAATAACATAGCATTGGGCCAAGTG
ATTTTACATGCAAGCATCATTATTGGGAAAATTGTTTTGTATTTTTCTCAACAATGAACACATTTAATAT
TTTTAAATGTTTTTTTAATAATTTATATAAAAAACATAATTTTAATATATATTTTTTATTAAAAAGCACC
ATTTATCAATCAAAACATGTATCAGTAGAAGAAGGATTAGATCAAACCCAATCAAAATCAATATTTATTT
ACATGAAAGCATGAAAAATACAAATAAAATATGTACATCATACTTAAAATGTTTTTTTTTTTTTTTAAAA
AAAAGAATACAATTAAAAAATATAAAAAACTATTCGAACAAAATTAATCGAAATATGTTCGAGGAGAGGA
AGGATTGAATCAAACCCAGTCAAAATCAATATTTTTGTTCATCTATTGATTTATATCAAAGCATGAGAAA
CACAAATAGAATTTAAAAAAAAAAACTGACAAAAGGATATAATTGAAAAACATAAAAAACTATTCGAACC
TAATCCATAAATTTCACATATCTAGGAGGTTCCCTCAAAATTCTAAACAAAATCCATATCCCTTTCGGTT
TCGGATTGTTCGAGTGCCGTGTCCTAGCAAATATTAAAAATTCTTGCGAATTTGTCCCTTGCCCCCCCCA
AAAGAGCAAATTCCCCACTTGCCCCAAACTAAAAATAAAAGGTTAATCTTAATCCCACGGCCACAAGGAC
TCATTGGACAGTATTTAATTTCCAACGTACTTTCTTGTAAATTTACAATCCCATCTGATTACTCCTGGTT
CCAGCGAAACCCTCGTCTATAAATACATACTGTCCCTAACCTGGCAAAGTCTCTCTCGCTCTCTCCTGCT
CTCGCAATCGCTGCTTCTTTGTCTGTCTTTGGGTCGATCCGAAAGAGAGGAGCTCTTCTGCGCAATC
Sequence ID: 135
Sequence Length: 3135
Sequence Type: DNA
Organism: Poplar sp.
CCATGACAATAATACATCACCATATAATAGGTGATTTGATAATAAAAATTTGAGACTAAAAAGTTTATTT
TTTTTATTGTCTTAGATTTCTTATGGTTACTAATATGATGGCCACTGGAGGCCTACATGGTCATTAACTT
TAGGATCCGTGAGATTAGTCGAGATGCGTGCAAGCTGATCTTGACACCCACGTTAATCTAATAAAAAAAT
AATACATGTATAAATTAGTATTAGACCGGATATATAATATTTTAAGATGTTATTTATGATTTTTTTTTTT
ATGATAAAAACATTGTTAAGCAACCTTCCATGTTGACCGATATTAAATCTTTATCCAGATGATTTCAAGA
GAGCGTCCTCAGTTGATAAAAATCTAATACTTTTATAAAAAATTAATAATTTTTCTTAGATGTTGACTAA
AAATTTTGAATTTTTTTTTCCTTAATCACGAGGACAACCCTAAATTCCCCATTATAAAATTACAACTCCT
GAATAGGAGCTGGAGAAAATTTTGAAATTAACAAGCTAATTATTTAAAATAAATTTTTTTTTTATTTTTC
TTATTATTTAAAATATCACATGGTAACATTTCAACCAGTGACTTCCTCAAAAGATTAAGATTACAGGGTA
TTATCATAATCAAATGCAGGACCACTTATGATCACCATCAAATTCTAAAGTTAATCAATACTCTCGAAGA
TTAGAAGAATAAAACTTGAATTTAAATGCTAGTCAAGACATTGTCACACTCAATATGTATGGACTTTCGT
TGGGATTGAATAATTGCTTTCTCATTCTTAAAAAGTATATTAATAAAAAGAATAATTTAATTCTCAACGT
TACTACTTATATTGGGAGATAGATGTGAGCGATAAAGCTTTCCATAAAATTATTAATCCAATATTATTAT
GGTATAAGTTAAAATAAATGATTTTTTTACTGTTTATCACCTCACAAAATATTATTCATTTTAATAGTTT
TTTGCTTTCTAAATCTATATTTATATTTTTAATTTCATACTTCAATATTTAGTTTACTGAAAATTAAATT
TCATAATTTCCTGTGGTTTGTTTTATATTTGGTTAGATCGGTCTCATAACTAGTGTCACGAGTTTGGCGG
ATTGACTCGAGGTTTTTTTTTTTGTTTTTTTAATTGATAATTTTTTATTTTTCAATTTCATCATTTAAAA
TTAGATTGATTGGGAATTAGGCTTTATGATTTATCTTGATTTGCTTTTTATGAGGTTATCTTGGTGACAT
GACTCAAGTCGCATGTTTGACGGGTTAACCCAATTGACTCGAGTTTTTTTGTCTTTTTAATTGGTATTTT
TTTTTATTTCACCTTTTAATGTTGGGTTGATTATGAATTAAGCTTCGTGATTTATTTTGGTTTGATTTCT
ATGAGGTTATCGCGGTTTTATGACTCGGGTCACACATTTTGTGGGTGAACTCGAGTAGACTCTTGTTGTT
TTATCGTGTCCTGTTTTTAGATTGATTTTTTTTTATTTTCAACATTTAACGATGACTTTATTGAAAATTA
GGCATCATAATTTGTTTTTATTTGATTTTTATAAGGTTATCACAGTCTCATGATCTTAGTCGTGGGTTTG
ACAGATTAATTTGGGTTGACTCGAGTCATTTTTTTGCCTACTTTCTATAAGGCTACCTCGATCTCATGAC
CTGAGTCACGTGTTTGGCAGGTTAACCCGAGTTATATTTTTGTTATTTTTAAAAAAAAATCAATCTCATT
CTTCAACATTGGGTTGATTGGTAATTAAATTTTATAATTTGTTTTGATTTACTTTATATATCATCATGGT
CTTATGATTTAGATCATAAATTTGTTGAATTAACTTAAGTTGACCCAAATAGATTCAATATCTTGTTGTT
TCAATATTATAATTTTTTTAAAATATTGTGTTGAATTTTTGTGAATCAAGCTATGTGTTTTACTGATTTT
TTAAGTTGCTTTTAAATCCAACAAATTGATTATGTCACATCAAGTAAACCTATACACAATTTAAAATATT
TTATTAGAAAAATGTTAGAAATGACTGGATGTTTCTTTCTTATGTTAAAAATAGTTTTTTTGATCACGTG
TTTTGCAGATTAACCAAGATAGACTTATGTTATTTTATTGTGTCCTCTTTTTAGATCGATCTTTTTTTTT
CATTTTCAACCTTCAACAATGAGTTTATTGAAAATTGAGTTTCATAATTTGTTTTTTTATAGGGTTATCA
TAGTGTTATGACCTTGGTCAAGGGTTTGGTATTAATATGGGTTGACCCGAGTTATTTTTTTTATTTACTT
TCTATAAGGTTATCCCAATCTCATGACTTGAGTCATAAGTTTGGCAGATTAACCCGGGTAATATGTTTTT
ATTATTTTTTAATTAATTTTTTTCATTTTTATTTTTTAACATTGGATTTATTGATAATTGAATTTCATAA
TTTATTTTAATTTGCTTTATATGGGGTTATCATGGTCTTATGATCTGGATCATTGAGTTGACTAATTAAC
CTAAAGTTGACCCGAATCAATCTAATATTTTACCATTTCAATATTATTTTTTAAAAAAGATATTGTCTTG
AATTTTTGTGAATCAAACTATATTTTTACTAATTTTTCTAAGATGTTTCTAGATTCATCACGTTGATCAT
GTCATATTAGATTAACTTATATAATTTAAAATCTTTTTATTAGAAAAATATTAGAAATAATTGGATGTTT
CTTTCGCACGTTAAAAATAATTTGATCCGATCCGTGACGCAGCACAGGTGAACAATCTAGTATTAACTAA
TTTGAAAATATCTTCATCACTCTATTCTATTTATTTATTTTTTAAAAAATTAAATATAGGGATGAAATTA
ATCTCAATTAATTAAGAAGCTAATAATTAGAGAAGGGGGACACCGCATAATAAAATAATCCACGGTGGGC
AATCGCGTTGGCTGTCGTCAGCGTAGTTATAACGCAACCTTCTTTTTTTAGCCCACTTTCATTCATCTCT
CTACCCTATATCTCTCTCTCCCCTTCTCTTCCTCTTCACGCATCCTATCCCTCAACCCATAACTACTAGA
AACTTGTCATATCAATCAAAACAGCGGCACCACCACCGCAAAAGCCAGAAACAAA
Sequence ID: 136
Sequence Length: 3278
Sequence Type: DNA
Organism: Poplar sp.
AAAAGTTCATAAATAAAAAAATAATTTAAAACTAAAAAAATAAAAATATTCTAAAAAATACAGTTAAACA
ACAATACTAAATACCACTGACATATCAATTAAAATTAAATCTTATTTTTTAAAAATATACTTTATACGTC
CGTGTCATAAATTTAGAATATTTTATAATTTCTTCTAACAATGATTGTAGTCTTCTTTTTTAAAAGTTTT
TTTTTTCTTTTTTTACAAGAAAGCAACACAGCTGAAGGGGGCATGAGCTGGCATTTTAAAAAAGGATAAC
AAGCGTCTAATTGTCGTTGGATACACGTTCTTTATCCCTGTTGTCGAGCTGGAGCACATCATTGATATTT
ATTTACATCAGAGGAATATCAATGGAGCCACCAGAAATATGTCAAATTTATTGATTAATTATAGCTCAGT
CCTTGTAGTTTATAAAAACTAATCAATTACTCCTCGTACTTTTAGAAATTATACATTTAATTCTCATGTT
TTCTTTTAGACATTCTTGAAGGGGAAAAAAATGAACTGATTTGGTAAATGTTGATAATTAGATTATTTAA
TTATTTCTACTACTATAGAGACCAATTAACTAGTTTTAATAAACTATAGTTATTAAGTTATAATGACTCT
TAAAGCTATGAATAAAATTTAAATGAGTGAGTTATAACCTTATCCTTATATATTTTTCAAACAAATCATT
CAATACATGTAATTTTTAAAATAATTAATTAATCATTTCACCATATTTGATTGTCATGTCAGGGATAATA
ATAGAAAAGTCGATGTCAACATAAGGCGAGCATTAAGTTATTCAAATTCAGCTTCAATAAATTCATTTTA
GTTTTTTAGGTGAAAAACATTCTCTCAACCAAATTGTGGTTCGAATTTTTAATCGCTTGAAAAAAATAGA
ATGCAAACTTTTTTGTATGAATGCTATGATTATTGGTAATTTTTATCATCTTTCATGTTTGTCTGATAAA
AAAATAAAAAAAAATATAATGATATCCCAAAAAATCAAAGTAGTTTTAAAACAAGGTTTATATGTTAGTA
ATTCGTTAATATTTTGGTTTTCACAATATGATTCATTCTTTCTAACTAAGAAGGTTGATGATTGATGGTT
GATATCTAATTAATACTTGTAAAAGATCTCCAATAATAAATTTAGAAACTCTCTAATAATTAAATAAGTG
TTTTATGGAGAGAAAAGATAATGATATTTTTTAGAGAGATTCTAAAAATAAATATGTAATTAAGAATGGA
AATTGAGAACCTAGTTCATTTAAAATAATTATTTAATTAAATACTTATTTTATAAACTCTTCGATACTTA
AATAAGTATTTTCCTGAGAGAGAAAACAATGATATTTTTTAGAGAGATTTTAGAAATAAATATGCAGTCG
ATAATGAAGATCGAGAACCTAATTACTTTAAGAATCCAATAAGTCTTTATAGCCCATGCTCCCTGTCATT
TGGTGCTAAAACATCTCTCATGTGTCAAAAAGAAAAAAAAATTCTGACTTATCAAGGAAAAAATATTTCT
AACTCATCAAATGAGGGATTTTTTGATTTATTAGAGGAAAAATATCTCTCTAATTTGTTAAAATCTCATA
TTCCAACAAAGAGTCTGATAAGATTGCCAAAGACATTTGTACCTGAACTCAATGCTTGACTGAGATAAAG
GGTAATGAATTTGGCAACTCACAAAACTTGTATATGTGTAGGCTTAATTTTTGACCGGGCCCGAGGATAT
TGGGTTATTATCCTGCCTTTAATTCATTTAAAAATATATTCAAATAAAAATAAAATATGCTGATATATCA
AATTTATTTCAAAGTTCCAAGCGGTAACATCAAATATCTATGGATCCTAAACATCAAAGTAATATGATGG
ATCCATATATCATAATGAAAATTTAATTTATTTAGAAACCATTTCCAAAAAACAAAAAAAAAGAAGGTGA
AAGACTAAATAAAAGGTTAACTCAATATCAAGATAAAAAAGAAAAAGAAATGGGATGTGATTTTTTTTTT
GTGTATATTGATATTTAGAGTTTATTTAGTAGTATGGTTATCCTGTATTTTGTTTTATTCATGATCTTGA
AAGTATCTTATTAATCAAAATCTATTATAGTTTTACATGAATTCTATTAAAAATTCTATTATAAAACTTT
ATTATAGAAACAGCTTTTTTTTAAATTTAATTTTTTAAATCACAATGATAGAAGTTACTACAACGCTCAA
ACAATGATGATGATTGTTGTAGGCTGGCGGTTGAGATGATGAATATACATGTGTTAATTTGTTGATGAGC
GAGGAATTAATCCTCTCAAAAAACCGTAATTTAGAGAAGCATGGGAAATATGCACGATTTAATTGGGCTT
ATTTAAGTTTTGCAGGGCTTCTTAAGAACTCGTTTGGAAACGCGGTTGAAACCGCGTTCCCATAAAATTT
AAATTTTTTTTGTTATAATTTAATATAATTTATATGTTTTGGATCGTTTTGATGTGCTGATATTAAAAAT
AATTTTTAAAAAATAAAAAAAAACATTTGTATACATTTCGGCACGAAAAGTTATTTGAAAAGCAATCGCT
ACCACACTGCCAAACACACTCTAAAAGTCAGCTATGCACATGTATATTTCTATTATTATTATATTATTAT
TATATACAAAAACATATCAGCATATATGTTTTGGTTTTCTATTGAAATGATCCACAATTTTGGGCAATTG
AACCAAACTAAAACTATTTTAGATAAGAAAAAAAATTCAAAATTTAATTCAAGATAAAAAAACAAATAAT
TAGATGTGTAAAACACATGTTTTGAGCAATTAAACCAAAAAAAAAACTAAAAATATTTTAGATTAGTAAA
AAATCCAAAATTTAATTCAAGAAAAAAAACAAATAATTATTTCATCTCAGAAAATACATATAGGGATTAG
TTCATCTCAGAAAAAGAATATATAGGGATTAGTTAATTATTTTTTAAAAATTTAGCAACTAAATTATTTA
TGTCTTGAGAATACAAGAACTAAATTATAATTTACCCAACTTAAAATTCTGATTCCATCAACCGAGAGAG
AAAAGAGAGTAGAGAGAGAGAGAGACGCAAAACATAGAGAAGCAGCAGTCCTCCATTTTCTCTCAAAAAG
TAAAAGCCTGCGTTGAATAGAAGAAAGCAAGACCCAACAATCTCTCTGTCTCTATAAAATCTTGTTCTAC
CCTTGTCTTGCCTTTGTTATTCTTGCCTCGACCGATCCCTATAAGAAGAAGCACAATC
Sequence ID: 137
Sequence Length: 1252
Sequence Type: DNA
Organism: Poplar sp.
CGAAATCTATCCAACAAGGACTATATTTTTCATGGTTTCTTAAGCGCGCAACAACAAAAACTGAATCTTTTGTT
ACGTCATCAAGTGATCTCCTTTTAATTTTTTGAATAGAACACAATTAAAATAAAAATAAAAACTGAATTTTTTT
TAACTGGGCCGGACCCGACAATAACATAATTGGAATTGCGATCAAATTTCACAAATGCTACATCATTATAATTT
TTAAAAAAACAAAAAAAATTAAACTCATTTTCATGGTTTTTCAAAAAAAAAAAAACTATTCACGTGATTTTAAA
TTTAAAATAATTTTTAAAAATATATATTAATATTGATGATTTTAAATTTAAATATTGTAGAATTAATTACTCCT
ATTACATCATGAAATAAATAATACTTTATATAAAATATTATTTATTATTCCATTAAACTATTTATAATTCCATT
ACGTACAAAATTCATCCGATAAGGACTATAGGTTCCATGGTTTTTTGAGCGTGTAATAAAATCAGGTAAAATAT
CATCAGAAACAAAATTAGAATTGCGATCAAATTTCACAAATACTCCTCATCATGCGATACTCTTTTAATTTATG
TAGTGTTATTAAATAATATTAAATATTAGTTTTTTTAGTAAAACATAATTTTAAAAAAATTAATTTTTTTTATC
GGATCAGACGCGGCCTAATAAATTTTGCTTATGTGAATAGTGAAAAAAAATTCACCTTGAACAGTGCCAGATGA
ATGCTTCTCTTCACCTTCTCGCATTGCAGGTGAATTATCACCTTGGCACTGTTCACTTTTTGAGTGAACAGTGC
CCAGGTGAATTTAATTCACCTGNCATCACTTGGNATGGGCACTGTTATGAACAGTGCCCAGATAAATAAAGAGA
AGCAGCCGGAGAGAAGCAGCTCCCAGCTGCTTTTGCAAAACCATGGTTTGATGGGAAAAATTGTGGGGCCCATC
AAATGAAAATCTGCTTTTCTTGCGTTACCAAACACTTGGTTTACGCGGTTAGCTTTAAAAGCAGAAGCTACCGC
GTAAACAAACACCCACTTAGTCAACCTGAAGTAAATTAGTGCTTCCTTAATTTATGCAGAAAAGAAAAAAAACA
CAGCACACCAGCTCGACTTCATTCCAATGGCTTCGTCTCCAACTCTCCCTCAAGATTCAACCCCAGACCCAAAT
ACTCTCTCACAAAACCCTAACAACGCAAACCCAGATCAAATTCAACACATTACCTGATGATGATGATG
Sequence ID: 138
Sequence Length: 3250
Sequence Type: DNA
Organism: Poplar sp.
TCTACATTATACTATACTTTAAAATAAAAACACTTGGCTAATTAAACTAGTCTAATAATTATATGTTTGT
GTCCTTTAAAGATATTGAAAATTTACATTATTATTAATTTTAGAACATATCATATTAATTAAAATACATG
TAAATTTATCTAAATATTCACATTAATAATAATAATAATAATAATAAAAATCATTATACATGTAACTTGG
AGAAAAATTCATTATATTATAAATATAAAGTGTTTTTACTTTAAATAATTTAGCATCATTAGAGCATTAT
TTTTGTTGTTTTTTAATTAAAAAAAACTGTAGGGGCATTGTTGGGTCTGGTTTGACAGCTAGGCCCAACA
CTATTAGGTCTGGCTGAGCCGTCAAATATTTTCAATTTTATTTTTTTATCTCTTTAGATTTTTTCTTTGC
TCTTATTTAATTTTTTTTCATTCGATAAAGAAAAAATAAATTAATAATATTTTTTTTATATTATATAAAA
GTTGAATAATGATAATTTTTTTTATTTAAGATTGAGTTGGGTTGCAATTCTTTTTTTTTAGCGGTTATGC
TGCCACCAGGATTTAATAGTGTTGGGTCCAACTTCCAGTAGGATTTTATAGCATTGAGTTCGGTTGTCTA
GACTCAACAACTTTTAAACTTCTTGATTGTTAGGTCCAATGCTATCGGGTCGTATGATTTGACCACTAAA
TCTAATAATATTGGATCTGACATTACCATCAGACATAAAATACATAATATATTTTATATACACTTCATAT
TTTTTTATATTTTAAAAAAATTATTAATGATCCGTTGATAAGTATGGGAAAATATTCTAGTGTGTATGTA
TATTTATATAACCTGAAAGTTTAAAGGTGTTAAACTGTTTTTATTACTAGTTGGTGGTTTTGATTGGAGA
TCGAGCTGCAGAGATCAGCAATGTAGACTAAGTCCTCGTTTATTGTATGGAAAGTAATTTTTAGAAATTA
TTTTTAAATTGTTTTTGTTTAATAAATAAAAAATATTTTTAAATAGAATATACTTTTCAAAAATTATAAA
AAATTGAAAAATATCTTCAAAGTGCAAATACTGTCTAGATAACCAGTTCCGTCTCAGGTAGACCCCTCAA
GCCACCAAGTGAACAAGAGAAATTGTGAGGGAAACAAGAAATAAAGTTAAACAAGAACTGACATGATTAA
AGTTCAGATTAAAAGATTTTTTATTTGTCGTTTACTTGATCTAATGTGGAAAGTGGAAATGGAGATAGTA
GATAGACTCCCCCGCATTAATGTATCTATCCTCATATAAAGTATGAATAATTAAAATATTTAAAAATATA
TTAAAATAATATTTTTTAAAAATTATTTTTGATATTAAAAAATTAAAACAATTTAAAAATAAAAAAAAAC
ATTAATTTTAAATAAAAAAAACTCAAACACTATTTAAAAAACGTTTTACACGCATCTAAACTTTTTTTAT
CACTTAATTTTTATTTATTTATTTTATTTAAAAAATTTATGAAATTAGATTTTATTTTTATTAATTTTAT
TATCTTTCGATCTTTTTATTTGTTAAATTTGATTTTTATTTTTTATTATTATTTTTTATTTTATTTAAAA
TAATTTATGAAATTGATATTTGTTTTTCAATTTTATCCTCCAACTTTTTTATATATTAGATTTTGTCCTT
ATTTTTTTAATAAAATTCAAAACAAATTAATAAATTATTTTTAGCTCATTTTTTCATGACATAATCAAAT
ATTAAAAAATATTTTTTAATTTTTTTAAATATTAAAAAATAATTAATTTTTTATAAAAACTACTTACGAA
AAGTAAATTATTCTCCAGCAAACAAGTTCGACAAGCAAACGGTATCCGAGAAAACCTCTGTAGTTTTGTC
TTGGCTTCTCTTAACATTCTTTCTACCCCTTCTACAACGATATTTTCATAATTAAACTAAGAAATAAGTG
AAGACTTCACTCCCTTTTCATGACATGTATGGGGTTTTTTTTTTTTTTTTGCATGAGTGTGTTCTAGTTT
AAGATATATTTTTTGCACCCCTGGCCAATACGCCCACTCCCCGATCAGTTGATATCATATGCATTATTGA
ATTATATTGCAATATCTTTAAACTACGCTAGAAATTAAATAATTGACTTTTTGATTAATATCAATATCAA
GAGGCCATGAGTGGATGTGAGTAAAATCAGGAGAAAGCGATGCAGACAAATTGCGAATCCCTCAAAGTTC
AAAAAAATCCATGTTGGTTTCAAAAAAGTCCGAATTATTTTTATAAATCATCATCCTTATAATAGAAGAA
CTTACATTTTAGTAATATATGCCGTAATTGTTTTCTATACATCACATTCCTTATACTAGAAAAACTAACA
ATTTAGCACATAATACATAACATATTAAATTTTTTTAATATGTTTGAATACTTTTTTATTTCAACGTATT
CATTCCCTCATCCCCAAAGTAAATTATTTTAGTTTCAAGAATAATATAAAAGTATTCTTATAAATCTGGT
TCATCTGCGTTCTTAACATGATAGCCGGTTAGAAATTCAAATTTATTCTCTGTAAGTGAATGAGTAAATT
AATTATAAGGTAAAAGTGCCTCAGTGAATGACTCCATATCCCTTTTTTTTTCATGCTTTCAAGAAGAGAC
ATCTGGTTCTGGTATTGCCACGACGAAGTATTACTGCTGAATGGCAGAAGAAAACAAACTCGAGTCTTGA
TGTAGATAACGCTATCGGTGAGAAATCTTGCTGTGGATAAAGTTGTCGGTGAGAAATCTCGAAGTAGATA
AAGTTGTCGGTAGGGTTTTCTGTGTGCGATTGAAAATATTTTTTAATCACTTCGAAGTGATTGTTCGGCA
ACAAATGTGCATTGTAATCGTTTTTGCTATTACAGTTTATTTTTTAAAAAATATTTTAGTTTTATTTTTA
GTTAAAAAAGATAGATTTTCACATATAGAATAAATTATAGACAATTTTATACGTGGTTCAATATAAAAAT
AAAAAACTATTTAGTTTACCAGATGTTTTTTTTATTAGATATAAAGTAGAAATTATGGCAATGGCAAACG
GTATGTAAAAATCCCTGGATTTTTACCATTGATCACGTGACATGCATCTTGTTGAGTAGTATAAATACTC
TTGAGGGAACCCAATCAATTTCAAGTTGAAGAAGTGCTAGATACTAGTAGTAGTTGCAGACAAAGAGCTA
GTGTTAGACAGGTTCTTGTTAGACAGAGAG
Sequence ID: 139
Sequence Length: 3055
Sequence Type: DNA
Organism: Poplar sp.
GAGGAACTATATCCAAAGAACACACATGGAGAGGAACGAAAATCCAATTTATGATTATTATATGGACGCA
GAAAAGGAAAACAGAGACACCCAAAAGTACGCAGAAAATGATAATCAGGAGACCGGTGAAATAAACAATC
AAACAGAGAGCAATGTGCAAGAACAGGAGTAGACATGCGATTTATAAGATAAACAGAGGTTTCAAAAGCA
TAATTCCAAAATCGAAAAGGTGTTTTACATTGCCCTAAAAGAGTAAGACCTGTTTCCACAATATGCCTAT
GACGTCGCTCTACTGTGCCATTTTATTCATGAGTGTGAGGACAAATCAAACGATGATGAATACCAACAGT
CTAAAAAAATATGGATAATTTTCGGTATTCACCGCCCCAATCAGTTTGAACATATTTTATTTTTAATGAA
AATTGACGTTCAACGAGAGTCTAAAATTGATGAAAAACAGAGTAAACATCAGACTTGGCAACGAGAGGAT
AATACCATACATATTTAGTATAAGCATCAACAAAGATAACAAAATAACGATAGCCATCTGAAGAAAAAAG
AGGAGCAGGAGTTTTGTGACCCGTAGGTCCTAAAGACAAACGTGATGATTTTCCTAAAGGACAACTTTGA
CATTGAAAATTAAGACTTTTGTTGTTACAAATGATCTTATTTTTTGAGATTAACAATTGAAAAATACGTG
TAGTAGGATGACCTAGTCGACGATGCCATAAATCGGCAGAGACGGAAGTGCAGGGAGACCAATAGGCTTG
AGGAACTGACGTGACGGAAGACTTGGTAAGGGCATAGAGGATTTCATGGGTGTTGAGATCCTTGACATAA
AACACACGAGGGTGAAATTCAAAATAAACATTATTATCAAGATAAAATTTCTGAACAGAGAGCAAAGGTT
TCGTAATTGTAGGAACATGAAGAACATTAGATAAGGTGAAAGAACGATGTGGTGTATATATTTTTGTATG
ACCGAGATGAGATATAGGGAGGCCCTTACCATCACCAACATGCAAATTATCATTACCAAGATACGGTTCT
GAAGCAGTCAAGGTGGCAAGATCAGGTGTGACGTGTTGATTGGCACCGGTATCCGGAAACCAATCAACAG
AACCGGTTGAGGAGAGATTGCGCTACACCAGATTGGCAGTAGGTTGTTGGCCATAACCTCGCTGCTGGAA
TTGAGGGCAATGGGGAGTTGTATGGCCGAAATTCTGACATAGTTGGCAGCGTGGATTCTACCCCCTATTG
CGCTGCCAAGAGCCCTGCCTATTGTCACCGAAGGAGGAGTTTTGGAAGCTGCGGTGATCAGGTCTAGAGC
CAGCAAATCGGTTGCCTCTGCTGTTGGACTGGTTGGGACGCCAGCCACCGTTGAAGCGGCCCCTGCTGCG
GCCAGAATTGACAAAAGTCTGGCACTGCGCAACAAAAGCAGAAGATGGAATGCTGGGTGTGGGCAGCAGA
GGAGCATGTATGGCAGCATAAGATTTGTGAAGAAATTCATGTGTGAGGAGATGGCTGTGAAGATCTGCAT
ATGATAAAGGTTTAGCCTTGGTAATAAGACTGGTAACTAAGTCTTTAAACTCTCCCCGAAGACCACGAAA
CACATATAAATTGAAATCTTCAAGTGAAACTGGCCGACCAGCAGCGGCCAATTCATCAAATAAGGCCTTC
GCTTTTTGCATAAATTGAGTTACTGATTCATCACCCTGTTGAAGATCCTGAAGAGAGCTGTCAAGTTGCA
TAATATGAGAGTTGGAGGTACAAGCGAGAGCTCGCTTAAGAGTGCCCCAAGCTGAACAAGAACTTTGACA
GCCAACAACAAGATGCAAAACTTCCATAGATAGGGAGGAAAGAAGAGCACTTAGAATGAGTTGGTCCTGT
TGTTTCCAGTTTTGAAAAAGCGGATTTACCTGAAGAGAGATACCATCATGGGCAAAAACATGTGTTGATG
GACATGTGTTGGAGCCATCAACAAAACCAAAAACTCCTTGGCCTAGGAGATAAGGCATCATCTGCATACG
CCAATATAAATAATTGGTGTTTGTTAATTTGAGGGAGATGACTTGATGAGTGTGAGAAAGGGAGATAATG
CTTGCGGCAGTAGAGGCAGCAGAGGCAACAGACGTGGGCTGCAAGAGGAGGCGTTCACCAGCCATGGCAG
AGGAGAATCCAGGCGGTGACGCAGCAGAGGAAAGCACAGGAGGCGCTGCCAGAAAAGAGGGCTGCACTGT
TGGAGAAGAAGAAAGCTGGACTGCTGGTGCTGCAGAATTTGAAGATGTATGTGGCGGCTGTTCCATTGAA
GAAGGGAGGCTGGGAGGCTGTGAGAAAAGTAGGTTTAATTTATTTTAGGGTTTTGTGGCTCTGATACCAA
GTTGAGAATAATGAAATGTGTTAGGTTGTGCCTTAGCCAACCTTCCTATTTATATAGAGGCAGCAGAACA
CTGCAGTAACTGTAATGATTACAACATCCTATATTAATTTCCTATTCTGATAGATACATGATATACATTC
TATAATAAATTTGTTGTTTTTTTTTCTTCAACTTTACATCATTCTTTAAATAGAAAAGCTCAATTAAATT
TTTATTAATTATCCACTCACCATATTTCATTCAAGGTAATTTCTAGTAAGTGTTCACTCATATTATTTCT
TTTATTATTTTAAATATCTTGCACTAAGTAGACTCGTTTTACTGTTCTTTTAACACCAATGTAAGTATCA
TTTACCCAATAACAGTGGGAGTTTTTTTTTAAAAAATACGATGTCAAGTTCTTAAAGTGGCTTAGGAAAG
AGTAAAACTGACTATGGTGTCAAGTTTGACAAGTTTTCCGAGAACCTAGCTTGCAATACTCGAAGACAAA
GTTATGAGAGAGGTATTGAAACATCAAACGGCATCCATGATAGGTCCATAATACAGGGCTTTCATGTAAT
TGCATTAAAATAAGCTTTGATTGATTTCTATATAAAGGTACATGAGGAGCACCCCAATAACACAAAGCAG
TTGCATTTCTAAAGTACTATCCGTCTGCTCAATCCACTTCACACA
Sequence ID: 140
Sequence Length: 3146
Sequence Type: DNA
Organism: Poplar sp.
Other information: n = A, T, C, or G
TTTGAATTTAATCTTTCCGCCATGATTTTAAATTTAATTTCATCCAATTATGTTATAAAAGAAAATTAAA
GTTGAAGGATAAAAATTAAAAAAAAAATGATCGAAAAAAAAAAGTTGGGAAGTATTATACTATTTATATA
ACCAGTGAAGCAATCGTATTAAGCCCTGTTTTTTTTTTTAGTTTTGTAGTTAATTATTTTTACGTGCCGA
CAAGTACATCCTTTGAATGGCTATTCATTAATTTGTAGTCAAAGTCCGAGAAAAATACATATGATCTATC
AAAACCAAACAATAGTGCTCTAAACTTGTTCGTTTTTCCTGTGAGGCTGTGTGCATTTACTTCCTCCTAA
ATCCATGTTATTTTTGTTTTTATATTTTGAAAATGTTTTTGAAAAAAATTAAATTGTTTTTATTTTTATA
TTTTAAATTAATAATTGTTTAATATTTTAGATTATTTTGATGTAATAATATCAAAAATAATTTTTTTAAA
AAATAAAAAATACTTTTAAATATAACAATTACCACAATTCCAAAACGCCTCTAAATCAGGCTTCGGTTTC
GAAATCTTATCCAAGATTTATGTCTGATACAAAGAATACTGTTCAAGTCACATCGCTCTATAAACGAGTT
AAAGACGACAGAGTCTAGCTCATATATATATATATATATATATATATATATATAGTAGACAAATCACAAT
CGAGAAATTAGTATTAGTTGCAAATCGTAAATGTTTCGAGCAGTAACCGACATAGCATAGTTTGATGAAA
TAAACTTTGACCTGAATGGTTTTTTTCTTAAAATAATATCATAGTCAATTACTTATATATTTACATGTCA
AAAATCTCCCATCGCTTCTGAGAGGAGGTGTTTGATATAATTAATTTTTATTTCTGTTTTTAAATATTTT
TTTAAAATATTTTTGGTTTGAAAAATTATTAAATTATTTATTTAAAAAAAAAACTATATTAAAAAATACT
TTACACTAAAATCTCAAACAGCATGAAGATGGAATTAAAATGCCATGTAAATATAGCATTGATATTAGAC
AAAAATAGGTTGGATTGGAAGAACATGTCTCCTTATCTTTTTTTCATTTTAAAATGACAGTTTCATTAAA
AATCTGTCCTAAAAACATATCTATTTTTATACTTTTGTTTACGTTTCCACGACAAAATATGTTTTATATA
TATATAAAGATAATAACCAATCATAACTAAAAAATAAACAATTTGCCCCGGTGGTTTCGTTTTCTTTTCT
TTTCTTTTCTAATTGTTTTTGGTGATATGAAACTAACTGGCCTCATATACCTTGCCAGTGAGGATTAAAT
ATTTTTTTGTAAAAAGACATACGCGATTGTCTGGTTGGAAGGTGACTGTGAAGCTGATTTGATGAACTAT
AAAAAACGAGAAAGTGAGATTTTTGCGTATTTTATTTCACCTCTCAATAAATAAGCCATTCTTAGGAGCC
GTGCTTTTCTTTTATTATTAAAAACTATTGTCCAAAATGTACCTTAATCCCATCCTAAATCACTCGCCTA
GGAACAATTATTAAAGCACCTTCACTATCTAACTAATTCCCATGGTGGTCCATTACTATCTCGAAGAAAA
ACAAACCGGTAACATGAAAGATATAGCTGAATTAGTTAGATTTTCTTAAAAAATTATCAGGTTGAGTCTT
TTAAATTTCAGAAATACTAGAAATTTACATGATTATTAATTTTAGGATAATGAGAGATTAATCGAGGTAT
ATGTAAGTTGATCCGGATATCTATAATTAATAAATAAATAAAAACACATTTGTGCATGATATTCGAGGCT
CCTACCAAATTAATGTAATTCGTGCGTGCTGGCATCAGGTGCATGAACACAGTACACCTGTGTTCCATGA
TCTCGAACNNNNNNNNNNNNNNNNNNNNNNNNNNNNNNNNNNNNNNNNNNNNNNNNNNTGTCATTTCTTT
ATATTTCTTTCATAAATAGAATCGGTAAAACGAGTGAGGCTAACCAAGATTGGGCTCTCAGCTGGTCAGA
AACTTAAGTTTTGTGCTGGTTAAATTAAAACCTAGCTATGACCTCCCATGGACGTCGATAGTTTGGCTCC
ATTCAGCACGGGACAGCTTCTCTTTCAGGAGTACAGATTTTTGCTAGGGCACCCAAAAGATTGGACTTCA
ACAAAAGAACCGGGAGTTTGGCACGAGACCATAATGATGACATAGATTGACGCACGGCTTGTTATGTGAT
TTCAAATTAGGTCTAAAAAAATATCGTTTAATAATTTTTTTTTCATATTTTTTCTTTAAATTTTTCTTTA
GTTTAGATTCTATTAATTAGATCTATTTTAATTAACGATTATTTGAACAAGTTTATATATTATTTTTCAT
GACAGTTAAAAATTATTAACATTCAAAATTTATCAAATCATAATATTACAATTTTAAAAATTAAAAAATC
TGAATTTCTTAATTTGTGATTGTGTCGGATGTTAAGCCATATATATCTCTAATTTACTCCAAATAAATCG
TTTATGTGGCTGCTTCCAAGCTTTGTCTCTTCAATCCTCCAGTTGTTAGTCTGCTATCTTGTGGAATTTT
CAGAATAGAAATAATTATATCACAAATATATCTAAAATCACATTAAACGCGAGCCTCTCGATTATTTTCC
TTGTTCCATTACCCATATTTTAGTCTTTCACGCATTCTCAATCTTCATCCCAGCTTTGATTTCCTTTTCA
CCTTTTTTGATGCTGAAAGATGTGCCACATCTCCGAGTACAATAATTGGAGAAGGCAAAAGTAGCATACA
CCAGCAGGGCCATTGATAGTAAGAGCATCCACTAAGGAATCACAAGAGTTCTTACGACCCTTTCCTCGTT
CATCCCAGATGATTCCGAGGGCTCTGCTCCACTCGTACGTTCCTAAAAACTCAATACTGACCGTATACTT
ATATACTACCTTTTTTCCCCTGCGTAAAATAGGAATAATACTTCGAGAATATTTGACCCTTCCATAAACC
TACCTCGGCCTCTTTATTCATTACTACCATCATATGGCTATAAATACCAAGGCCCTTTCTCTTTGGTTTG
CACGCCAAGGTTTAAGGTTTCAATTTGAGACAGTGAGCTCCGTTGCAGTACTTGTGTTCTCTAGCA
Sequence ID: 141
Sequence Length: 3923
Sequence Type: DNA
Organism: Poplar sp.
AAAAATAACGTAAATTATATGATTTCAATGCCTTAATTTTTTCCAAATTCAATTTTAATACAAAAGTTTA
TTTTTGTTATTTTTTAGTCACTAGTTGAGAGAGGTGAGAGAAAGGTCGTTAAGTTTCGGCGATAGAAAGA
GAAAAATATCGTTAATACTGATTTAAACTATCAAAACAGTTGATATTTATGTCAAATGGTTCTATTTGAC
GAGAGGAGTTTATATGAAGTGTTTTTAACCTTTAAAGTTCATGAAAACGCTGATATGAGCTTGATTTTTT
TTTTTGATGAATTTTTAGTTTTTTTAGGCTATTGATAGGTTTATCACAATTTATATGGTGTTTTTGTGTC
AAAATGGGTTGAAAAATGAGGTTTGGGTAAAAAAACATAAGCTCTGATTTTTCGACCACTACAATGGCAA
GAATCTAGGCAAATCATATGACGCATTGTTTGCTTCAGTAGGCGACATGTTATTTGAATTTTTTTTTTTA
AATTAGACAACATATTGTCCACCCCAATTGCAATGAAAAAAAATAAGGGCCTAATCGCACAGGCCTTGAT
GAAATAGGCCAGGCCTAGCAGCGCTTTTTTCTTTCTAATTTTTCTTTCTATTTTTTTAAATTAATGCTTT
TTTATATGGTTTTTTTTCTCTAAATGTTTTTTTATTTATATTTAGATTAATATCTATTCTCTCTTTTATT
TTGTTTAGAGTGTCTTTTTTAAACGACGATTATTTTTTTGTTATACATTTAAATTTTAAAGTTTTTTTTT
CTGATTCATTTATAATTTTTTTTTAATCTTTTGTATAGATGAATTTTATTTTTGAAAATAAAAAAAAATT
ATTTTTAGATAATATTTATAATATGTGAACCTTGCATAGTGTTTTTTTCCTTTTTATTATATTCAATCAA
TTTATTTTTTGTTTCTATTTCTATTATCTTTTGCTTAAATAAAACATTATTTTATAAATAAATTTAGCTA
ACACAACTGGGAAAATATCCTGGCTTGTGAGATTAAATATCTTGAACTACATTTATCTCTTAATTTTTTC
ATTTTTATTTTTGGTTATCATTTTTTGTTTTTTTTTTTCATTTATTAACACACATAGTTCTCGATTTATT
TGATTATAGGTATGTATAAACTTTTTGATTTTCACTTAAAATTTTCTTAATTGCTAAAATGTGTTGAAAA
AACCCGTGTATATAATTTTTTATTTGAAAAAAAAATATTTGACCTGCGGCAAAGCACGAGTTGAGTATCT
AGTTGTATGTTAAGATCACGTTGAAATTTAGTAATGCCTCATGAGAAGGGTTAACTTCTTCTCCTTGCTT
CTTATACTTTATTTATTTTTCTATAAATTACTAGAGTTATCCCTTGATGATAAGTCTCTCTATCTTTTTT
TTTAAGTGTATAACATTCCTCAGTCATGTCCCTTTATCTTTATGAAAAAAAAATATTTTTAGAATTTGTT
TACCAACATCTCTTTTTATTGGTGATGGATACTGGACAAAGTCTTTTCCTTTGATAGTTGTTAGTACCTT
AGTTTTGGTGGTATTTGAAGGATTGATTATTAATTCATGATATATCAACCTAATTCATACTAGACTTTTA
GGTTTGCCTTTGAATACACGAGAGGTGACAGGAGGTCGCTTGTGTTTTGGAGACTTATCTCTTTGTTTAT
AAAAACATAGGTACTTTTATCTAGCTGCATTTGTTTCTTTCATCAAAATATGATTTTTTTATTACTAGTT
GATTTTGAGAAGAGATTTATCTAAGAGAGCATATAATCTTTCCCATAAGAAATAGTTATGGATTCCTCTA
ATTAGAGCCTTTGTAGCAATCGGTTCAAACAAGTATTTTACTTTGAGCATCTCCTTACTGAATCTTTTTA
AATATATCATGATGCTTTCTTCTTCTCATTGAGTAATGGTGAAAAGCTTAGTTTAACTTTTTTTGACAGA
TATACTTGTATTAAAATAATCAATCAACTTCGTATATAAGTTGAAGAAGTTGAGTATAGAACTAGATTCA
AAACTATATAATTGAATTTAAAATGCCAGTACAGACAAATGTATTTTGATTATAGTTTACTTAGCTTGTA
CTGATTTGTTTTATACATTTATCTAAATGCATCTAAATATTTCATTTCCAAATGATATCAAACATCTATT
TACATGCCTAAGAAGTAAAAAATTATTACCTTATAATAATGTGATTGATATAACTAATTAAAAATTCATC
ATTTATTGGTGCATATACAAAAAAAACATAATACAAATACAATATTAATTAGATAAATTATTTAATCATA
GTTATTATTAAGTTCACTCTATTTTACTATTTCTTTTCGCTAATATAAAATTTACCATACATAACCAAAA
GAAATTACTATCAAATTAACCACAACAAGAAACACTATAATTAGACTTGACTTGCCCTTATAAGTTGACT
TAGAAAATCCATGATTGAAGTCTGACCCGAAAAATACATTGACATGATAAAACTTAGTTAATCTGGTCGG
GTCGGTTCATGACCTGGTTGATTTAGCCAAATCTAATTAGGTCAACTCCATATTTATTTTAATAATAATA
ATAATAATATTATTATTTTAGTAAAAATAATTGACTTATTAACATGTGAACCGAGGCTTCTTTGCTAATT
AGCATGTATTATATCAACATAATAATAAAAAAAATACAACCACAAGAATAAACTAATTAAAGAGAAAAAA
CAAATTAGCCAATTCCATAATGGGATCACAAGATCTACCATTACCAAACTATACCTACGTGCCTCACCAC
ACACAAATAGCGCGTGGATGGCAAGACATTTTGAAAAAAAAAAAAAAAAAAAACCAGCAGTAATATACCT
TGAAAATTAAACTAATAGAAGAAAAGAAAAGAGAAACACATCTCACAGATGTACCAAGTTGCAGAGAGGA
GCAGTTCCCTCCCTCTATAAATAACCAAAAGCTCAAACACCGTTCTTCGTGTCAAGCTCTCCTCTCTCTG
TCTCTCTCTCTCTCTGTTATATTAACAAAGGAAGGAAGGAACAAGTCTCATCACCTTTCCTGTTCTATCT
CCGTAAGGTAAGGCGGTTCCTTTTATTTCTCCTCTTCCTTCATTTCCCATGCAATTATAATCCAACAACA
ATAAGCACCTGACAGCCCCCTATTGCTTTCCTTTCCCCCCTCTTCCTCTTGCGTTTCTTTCTTTCTCTTT
AAACGTAAGTTTCAATTGAAACAAAGCGTAAGTAGTATACAAGATCTGCTTTTACTGATTTGTTTCATGC
AAATATGTTTTTTTGTGTTTTCTCTCATGCCCATTTAGACTTTAACGATGTTTGAAGTGCCTGATTTTTT
CCCCTGTCGTTCTGGGATTCGTTGATCTCATTCTGATTCAATCTTTTACTTTATTGCATTGGATCTGATT
TGAAGTAGTAAGTGGTTTTTTTTGGTAGTGGTGTTTAAGAAGAAAAGTGAGAATGAATGCACGATACATG
GGTTATGCTTATTGTCGATAGAGCAAATGATAAAAACAGACAACAAGTTATGAAGTTTCAATCTTAGAGG
AAACCATTCCTTAATTCACGTATTAGAAGTTGATTGCATTTTGGGTTTCATTTCTTTGTTGTGTTTCTCA
ACATTTGTTGAGTGTTAGATCTGTATTTATTGTTTGCTACTAGTGACTGCCTGTCTTGTTCAAAGTAGCT
GAAATTTGTTCCAATTGAAAACTGTTTTCTATATAATGAACAAAGTAGCGGAAATTTGTTCTAATTGGAA
ATCGTTTTCTATGTAATGGACCGGATTAGTTTTTGCAGCTTAATTGTTATTTTAGAGCTTAATTTTGATA
GATCTATGAAAGTTGAATGTTTGGATCAAGAAAAACTGAATCTTGATTTTGGTGTGGCATAAGAAGCAGA
AAA
Sequence ID: 142
Sequence Length: 3115
Sequence Type: DNA
Organism: Poplar sp.
CTATTTATTGGCTGCAACAATTACTGCCAGTTTTAGGCAACAGCTTAAAGGAAAAACTGCTGCATTCTGT
CTTGAAATTTAGTCCATGAAATTGTACTACACCACCACCACCACCACCAGTCATTAACTTACTTGTATAC
TTAGTGTTGTGTTTGGCAATAATATAGCTGATTGTTATTTTGATAAAATTTAATTTTTTTGTTTTAAATT
TAATTTTTAGTATTTTTAAATTATTTTGATGTGCTGATATTAAAAATAAATTTTAAAAAATAAAAAAAAT
ATTATTTTAATATATTTTTAAATAAAAAATATTTTAAAAAAATAACTCATGTCCGGATATAAAACTCTAT
TTCTTATTGGTTATTGTTTGAGAGAAGTTTTCTCTTTTCTTTTGGGACAAGTTAATGAAAAGGGGTTGGT
AATTAGTCTTGAATTAATTATTATTATTACTTCCACATTAGTCTAATTTGGGTGATCTACCTATAACAAC
CACCTTTATAATTTCAAGACATACATGACCCCCTCTCTTAGCATCGCCATACGAGAAAATCATGAAGTGC
ATTGCTCTCTAATCATGCTTCTTCTTTTTTAAACGGTTAAAGATGTTCTTAAACTAATTTGTGTGTTAAT
AGAGATTAAATTTTTTATCCTAATGTTTTAAAAATGTATTAGCACGTTACTAAATAAATTTAGAGATTTG
AATTTTCTTAACATTAAATGCATGCTTGTTTGTGTGATCAACGTGCGGTTTGTCGTATAAACAAATGAAC
CCCAAGTTACCTCTTTAAATTTACTATTAGTAAATTGTTTGGGTATGAAAACAAATAAAAAGTAAAATCA
ATTGGTTTAGAGACGTGAGATACTGTTTTAAATGTGTTAATTTAACCTACGCAAAGATAATATTTCTATG
ATACAACTAAACTCTTTAAAGCTCTTGAATAATAAGAAGAAAAGACAGGTAAAAAATATATTTTTTTTCT
ATCTGTTTTATTTGTGTATAACAAGTTTATATAGATTTGAAATATTATGATAAGATTATATAACTGTTAA
AAAAATGGTTATATTTTAAATTAAGAAAATAAATGTTAAAAATCATTATAAAACTCATCTATCATAGCTT
CTTAAAAAGATAATTATATTTTGAATTAAAAAATTAACTGTTAAAATTTATTAGAAAAACCCATCCATTA
AAGCTTTTATTTAATTTACTACACAAGAAAATATTTATTTTCATTGTAGATGGATTTGAAACCACACCAA
CATTTGCTTCACTTTTATTTAAAAGCATCAATAGGTCTCACCAATGTAACGTTACACTTGCAGCAGTACA
AGTATAGTGGTTCCTCGAGTCGTTTAAATTTCAAAATGGTCAGGTTGGTTCGAAAAGCAAGACACTCAAT
TGGTTCGGATCGAGATCTATTTCTCTGTGACCCACTTCTTTGGTGACGATGATGATGGATCATGAGCATG
CTTTTTAATCGCCTAGTATATGTTCTACATTCCTTGCTGCTGCTGCTGCTGTTTTCTTTTTGGGCAGCGG
CGGCAGCGCAGCAGCAGCAGCAGCAATCTAGCTAGCTAGTTGTTGAATTTGAGGTAGGGGCTAACTCGTG
CAAGGATCGATGATTCCATCACCTTAAATGTTGTCAATCCCTATGGGAAATGAATGCTAGACGATGCCCT
GGTGAAATCTTGGAGAGCCCTGGATTCAAAATAAATCAACTGAAACCCAATCACCTGCCATCATCAACTG
CATGGATGTATATTTGGATTGGAACTACAGAGAAGATCAATTCTCTTGCATTGCTGGCCCAGAGCTGTCA
AGCGTGTAAAATATAATAGGTATTGATCTTGACAAAGTACCAACAAGTTAGGTTAAAATTAGGTGCATAT
CTGAAGCATTCCTTGATGTTTAACGTCATAGGAATGTTCATATCGTGACGGTTAAGGTATCCCTTTCTAT
TTGGAATTTAAGCAAAAAAATTCTCAGTTTCATCAAATTAAGTCGACATCGACTCATAAATTCCCTCAAT
GATATCATTCAAGCAAGAACAACTTGTTATCGTCAATCGACATCACACCAAATAATTACGATAACGAAAA
ATAAATTTGATTAATTAGCTGTACTGGACTTGGAATTAATGCATTCACTGTTTTTATCGGGGAAAAGAAA
AGGACTTCTAGCTATATAATTTCCAGTTGTAAATCATGAATTAGCCTAGCAAGCAGGTTGATTTAATACC
TCATCGACTCCAAGTTAAGATGAGTTTAAGTTAAAAAAATAAAAAATTCCAGTTGATATGTTGAAATCCA
GTTAAAATCTGTTTCTTCTTTTTACAAAATAGTATTATTTTGATTTTTTTTATGAAAAAGGTTAAGTTAA
CTCGGGTTAACCTTCTTGAATCACCTTAGCTCTGAATTGATTTTTAAATTGATTTTTATAATTATAATAA
AATTAATATAAAATAAAAATAGAGAGAAGAAAAGGTTGATATCTAATTAATATCCAATTTGTTTACCTTG
AAAATAGAATTTACAAGATTTTATTCTTCATTTAAGATCTTTATATATATATATATATATATTACAAAAC
CTTAATAATTTATTATCCTTTCAAGTCATGCCAATGATTTAAGACCATACAATGACTCAAACCATGTTAG
CAAGCAACCACATGAAAGGTTTTTCCCATCAACAAACTTGGGAAGGCCAACAATTTCTTAAAAGAAAAAA
AAAAAAAAGAGTATATTTCCCACAACGTTGAATGAAATCATTCACAACCCAACAAAAAAAAAAAATCAAT
TATTAATTCAACAGAGAGAGAGAGAGAGAGAGAGAGAGAGAGAGAGAGATCGAACAAGACAGCAAAATAT
GGAGACCCACAAAGGAACATTGGACAGCTGTTTGGCAACCCTTGTCTTTGCCACCCTGTTCTCACACTTC
TCTTCAAAATCTCTCTTTACCCCTTTATATATGCGCACCCACACCCACACATAAACACCTACCATCCACA
TTCCTACAAGCTGTTGTCTCCATTCTCTCTCTGCAACTTCTCTCTAAACAAATCTCATTTCAAAGAGGAA
TCGTAGTCTTTCTTTCTTTGCTAAACAACAAGAAA
Sequence ID: 143
Sequence Length: 3073
Sequence Type: DNA
Organism: Poplar sp.
TTTAATTGAATTTATTAAAGACTTAATTGCAAGAAAAATTGATTTTTGAAGTCAATTTAGGCTTTAATTG
GAAGAAATTAAAGTCTATGGGTCGAATTGTAATTTTTTGGAGTTAATTTGGTCAGATCAAGGGTTTAATT
GCATAAATAACAAAGTTTGACTAACAATTAAGAACTTGATTTAAGAAATCCAAAACCAAGAACCAAACTG
AAAAAGACATGTGAATGTAAGGGCTGAAATTGACCTAATTATGGGTTGAATTGAAGAAATTAAAAGTTTT
TTGATCAATTGAGGGTCGAAATGCACAAATTCAGAACCAAGGATTAAAATGAAAATAACAGTCAACTTTA
GGGCTAGCAATTGAGTTTGGCAAGGGTGAAATTGTATGAAATTAAAAGTTTGGAGGCATTTATAGGTATA
ATTAAAAGCAATTGAAAGAACAATTGCTTAATTGAAGTTTACCAAATCCTAAATTAAGAACCCTAAATGA
CTGTTGTTCAGACTTAATGAAGAGAAGGTGAACGACACATCATTCATCAGCTTGAATCTCTATTTTGACA
GTTTTGGCGGGTCGAATCGACAACTTCAAACGAATGAATGTGGAGTTACTAACTTTTAAATTGAGCAAAG
TTGGATCTAAATGAAAGGTGTGCACCCCCTCTACCAACTTCAGGTGGTTTCAGAAGATGACGATTGCATA
ATTGCTTCAATAAGACCTTGAAAATGGACAACTCAGTCAACCCTGACAATGCAACCTAATGCAAAAACTG
ATTTGGTTTTCGTGTGTTCATATATAAAGGCTCCTTATGGGTTCTTATCAAGGGTTTACATCATTTCCCT
CAAGATTAAGAGGTCAGAAATGGATTCAAGACCTCCCAAAACCCTAGAAATACCTTCCTCAAATAAAAAA
AGGCTGAAAAAGAGGGGAAGAAAAAAAGAAAAAATATAAGAAAGGGAAGAAAAATATCAATAAAAAACAT
AAGTGTAGAGGTCTAAAAGCCTAAACAAAAGTTTTCTTAAGCTTTGCAAGTTTGTAAGAGGCAACATTTT
CCAAAGAAAAGCCAAGAAAGAGTAGTTGTTCTAGGTATACATTGTTGATCCAAAAGTAGGTCCTTTGTGG
ACTGCTTTTGTTGTTTTATTTATTTATTTGGTTTTATATTCATGTTTACAAGCATAGAAAAGAGGTTTGA
AATGTTTAACAAATGATGTTGCATTCACCTCTTCGTTGCTATGATTTTATTTAAAGAAGCTATTTTGAAG
TGTTTTTTTTAATGTTTTTGATACATGAAAGTTATTATTTAAGTTTGTTTTTCATATACAATATTGTTTT
AGTTTTGTTTAACTTAAGCTATGCATGTTAAAACAATGGTGTTTATCCTCGGTTTACTAGGTCTGTTTAT
GCACCTAATGAGTTCAAAAAATCAATTTTGAATCCATGTTGCATGAACATATATGTTGCTTTAAGTTGAA
AGTTTTTTTGATTTGGTACCTCATGCTTGATTTTGATGCTTTGATGTTTTTTTTTTAGTTAGTTTTTTGG
TTCAAACATGTTTGTGTATGTTATTTTGAATTCTTGAGATAAAAAGAACATGTTTTAAAAACAAAAAATG
CCTGGCTTGGCTAGGAACTTTTCTAGGTTTTTTAGGCAATGTTCTTTGTGTTCTTGAGAAGAAAGTTGTC
TTAGCCTATAGGTTCAGACCAATTTCGGTCAATTTCCTTGCATATAACCCTTTCTTATATGATTAACCAT
TAATCCAGTGTTTATTTGCATCAATAACATGATTTTAACGGTTTTTAATCCTAGAGTTTTGGTTTAGAAC
CGAAACCTATTTTGATAAAATTGTTATGTTTAAGTGATAATCGAAGTAAATAGATGTCAGATTACCGATC
CTTGCATGATTTCCAACATGTATATGCCTTCGTTTTGATCAAATTTGGTTTGTTTTGGATTTCATGTTGC
TGGGTTTGTTCGATTTTCTTGACTGTTCTTGGTGTTATTCATTTTTTTTTGTGTTTGATTCATTTTGCTC
AAGTTCTTTTGATTTTTTTTTAATGTTCTTGGGTGTTTTATTTTTTTTTTGTTTTTGATTTTTTTTTTGG
GTTGTTTTCGGGTCAAACTAGGATTTCTAGCTCGATTTATGGTCAAACCAAAAATCTCGGGTCAACCCAG
TCGAGTCAACTTGATCGAGTCAAGTAAAAAAAAATGGGTCAAAGGTTTTAATACCCTATTTGGTTTGCCA
TAAATTTTGTTAAAGGGTTTTTGGGTTTTAGGGTGTGTTTGGCAAACACCCATTAAGCTATATTTGGTAG
TTTTATTAAAAAAGAAATTGAGTTTTTGTCAAACAAAGCCTAAAAAAATAATAAAAAAATATTTTTTTTC
TTGCATATGGCTAAAAATCCTAAAGTTATTTAAGCTTTTATTTTTTAAAAATCTAAAATATTTTGACATG
TTTTTTTTTAAAATAATATATGGATTTTACAACAAGTTTGTAAAATTTGAAAGGATTTTAGTCTACATTT
TAAAAATACCAAAAATTTTTATTTTAATATATGGGATTATGAACTTATACGAAAGATATATTCCTGATAT
TAAAATTAAAAAAGATTTTTTTTGTTTCTTTTAATATACAAGATTATGAATTTATAAGTAAGACATATTC
CTGATATTAAATAAAAAGATAGTTTATTTTTTTATGCTGGCATTAGAACGGTTTTGTTTTTAATCTGATA
AAATAAAGACATCCTTACTAAGAAGTACTTTTCTTATACCTTAGACACACAAACTCTTATTCTCTTTTTT
TACTGATAAAGAACAATAATTCCTTGTCCTTTTCACCCACATCATTCTCGACGATCCCGATCCGAGAGGA
TGATCGCCGTAACATTGTACAAGTGCAACAAGACTCATCAAATTGATTAGATCACATCAAATCAATTCCC
ACATAATTTGTTTTCAAAACAATTATATTAAAAAAGTATTAGTAGCACCTAGATATTTTCGGTGGTGGTG
GCTATAAATAAGAGTGCAGAGAAGAGGCAATATCCATTTTAAACACAAAGTAATATTTCCAAG
Sequence ID: 144
Sequence Length: 2537
Sequence Type: DNA
Organism: Poplar sp.
GAGTTGAACAAAGGAAGAAAAAAAGAAAAAAAAAAAGAGCAAAAAAGCACAAATTCACAGAGAGAAGGAG
AGAAAAGGGAGGAGCAACACAGAGACAAGAATTAGCCTTGTTATTGTCTTCGTCCCTGCAGAAAAAGAAA
AACCCGAAATCAATTAAAAAAAAACTCAGAGAAAGCCGGCATATCCATTACCTTCTTAGTTCGTTCATCT
CAAGAACTGAGAAAACAAAAGAAGAGAGGACAGTGCGCACAGCTAGGAGATAAAGGAAAGCAGTTGAACG
AAAGTCACCACCAGCATTGCAGCCATCTTCGCCATCTCCAGAAGCAGGTGCGCCATTTCCCTTACATTCT
CTGCATTCAACAACATTTGAACACTGTGCAAAGGTAATTTAATTACCTTCGCACTGTTCATGCACGCGTG
CATCCGGCTTTTGCCCAGCCGGGTCACTGGTTTGGGCCAGTGACCGGGCCGGGCTGGCTGGGTCTAGCCT
AGCCCACATGGGCTGAGCTGGGCCCAGCCCCAAAAAATAAAAAATAAAAAAATAAAAATATTTGCATGCA
TAAAAAAAATTTATTTTGTTGGTTTATTCATTGATGTCAGAGTCAAGAATAAAAATACTAATTTAAATTT
ATTTTATTTAGTCACATAAAAAAATATATGTGCATGTATAAAAATAAATTTATTTTATTGGTTTATTCAC
TAACACCAGAGTCAGGAATAAAAATACTGATTTAAATTTTTTTTTTTTTTAGCCACGTAATATTTATCAA
CGCCAGAGTTGGAAGTATACATAGTTGAATACTCATTGACGCCAGAGTCAGGAATATTACAAGCAAACCA
TCATAGCATAAACCAACAAACTTTTAGCAATTTCAGATAGAACTAGCAATGCAGCCTGCCTCAGATAGGA
CGTTTAAGGGTGATAATATCTTTTTTTTTACGTAACCAGTCCCGTATCATAGAATCTCTGCTGACCACTT
AGGGTTCCTAGTGACCATAATATTAGGTGGCGACTCCTTAAACAAGACCATTCCCCATTAAAAAATAGAA
TGCAAAAAATCCATTATTTCCATTAAAGATATTTTTAAGGTCGTCATGATGTTGGGTTCAACAGAGCTAG
GTTTACTGAGACTCCGTTCTCATTATAAAAAAATAGAAAGAAGTTGGTTCCTAGACAAGGTGCTATCCTA
GGCTTAGAGCTGAGCTAGACCCCTACTTCGTAATGATTTAGAGAGCTCCGAATCAGGCTTTCAGGGCGAT
CAGGGCAAAAGAGAAAGTCCAATCTGACCCTAAGCAGATGCTTTTGACATCTTATTCCCATTCGCTTTCA
GGTTTGACATTAGAGCTCTCGTTAGCTGTTCAAAATTAAGCTGATTGACTTAAGGTGAGGAATTCGAGAC
TTTTAAGAGAAAAAAACACTATTGATTAATATTTAAAAGTGCATTTTTATTAAGGCTTTATATCATCATA
TTGCACTAAAGTATCATTAAATTCCCTAACTAAAGCATGTTTTATAATAAGAAATCTGATAATATAGTAT
ATCTTTAATTTATGGTAAATGTTTATTTTGAATGCAGGTATATCTCACAATTCAAAGGATGGATTGATAT
GATTAACTATTGAAAGTGAAGAGACAAAAAGAGGACTAAGCTAAGAGAAGAGATGCTGGTTTAATTCCAA
ATTGGAACACCATTCGGTTATTGGATCATAACTGGAGTTTTAGACCTTAGATTTAGGTCTGTTTTATATG
GATGGAAAGCTAAGACATAGATCTAAAACTTTTATGGGAGTCCAAGAATCAATTCTGCTGTTTTCAAGTT
CAAATCTTAGCAACAACAGAGAAGTCAGAATCTATCCTGCAGCTCAGACATTGTTCGGTGTTCAGCTCAT
ATCTTGAGTTCTAGAAGTCCAAATGAGCTCTGGTTTTTTTTGTTGGAAATCTGAGACAATTTCCCACAAC
TTTGATAACTACAATGGGCTCTAATTCTGATGTTAGCAATGATGTTTTGGCCAGCCAACAATATCAATAA
CTAGCCGACAATGTCAATAACCAGCCACCAAATCAATGGTTGGCCACCAACTAAATAGTTTGTCAACCAA
TCAATAGTTTCAAATTTTAGCCTATAAAAGGAGGCATTTGCCATGTATTTAGTCATATTATTTTTCAGAT
CAAAATCATCTTCTTGCTTTCTCTCTTTGTAATGTTCAAATTTTCTTTCATGCTATATTAATCTCTTGTT
TATGCTTTTTTCATTTCCTTTCCTATACTTAGTTAACTTATATCATGTTTATGTTCTTATATTGTTTATT
TATGTTTTTCTTCTTCAGTTTATGTTTAGCTAAGTTAATTATGTCAAGGTGAAAAGGTTTCACTAGTGGT
GTTAGAATATGTATAATATAAACTCAACATGGACTTTAATGTTTATATCAAAGAAAGATTGTTATTAACA
TGTTTTATCTTTTTATCTTACTAATTTTTAATACCTTGCTTGTTAAATGGTTAATCTAGATTTGTGTTAT
ATAACACTTGGTACATC
Sequence ID: 145
Sequence Length: 3334
Sequence Type: DNA
Organism: Poplar sp.
CGAGCGAGTTGAATATATCATGTTGAAATAATTTTTTATATAAATATTTACGAGTTGAGTTTATATTACT
AATTTTTATGAATTTATATTTTTAAAGACGGACCTACTCACCTACAACATAGACTATAGGTTCCATGGCA
TTCTTAGGTATAATTTTTTTACATTATACCACCATTAAATTCTAGCTACCTCTCCCATCTCGGCCTCTAG
TAAAGAAATAATTTTTTTTTGTTGCGTTAAAGAAATTTAATAGGAAGTCCATTGACAAGAACTTTCAAGC
CAAAATATTTCTAAATAAAATAAAAATAAATGCTCAATTTATAAATTATCAAGTATAAAGATAAAAGGAG
TATTGGGTATTCAGGTTTGGTTCGGATTGACCGGGTTTCAAAAATTTTGATCCGTCACTCAACCCTTTAT
ATATATCTATTTAATTTTTAGATTTTTTAATTCACCATCATATGTAATATCTATATTATCTGACTTTGAT
GAGTTGAATCGAGTTAGATAATATAAATATTACAGACGAGTTAGTTTTTTTAAACACCGTTGCTGATATG
ACAAGTTGCTTCGTAGAAATGTTTGATAAGGAAATTAAAAATGAAAATATTTTTGGGTTTTCTTCAATAT
TATGTCTAATCATATGTATAAACACCAAGCTGCAATCTTAATTTAGTAAAATAACATTTATAAGGTTAAT
AATTAGTAGAAACGTTGACTGTCAAAATTGTTGGATATTATACATAAAAGAATTTTCACCCTCTCCCTTT
CCTTTTATGAGGCCAGAATCTTTCCAACTTTGCTATGTTTTTTTTAATTTTTAGATTTTAAATCTTTTTT
TGCCAATTAGAGCATATGATTGACAATTATTCTGGTCAAAATTTGTGTTAGTCTACAAACCGTAGTTATA
AAACTGACATAATCTAATATTCAATCTACGGGAGTCTAGGACTGCCTCAATGACCGAATGAGGTGCATCT
GTTGCTTAATTAACATCTCAAAAATGCAAATATTTGAAAAATCTACATACGATCAATAATTAGTTGATGA
CAGTGATTATTATTATTATTATTATTATTATTTATTATTATTATTATTATTATTATTATTATTATTATTA
TTATTATTATTGCTTATGCAAATTATAAATAGATATCCCTCTTTCATTTTGAGTAGAAGAGCTGGCTATT
TAATTTTAGGTAGAGAAAGGATCTCTCTGAGGAAAAAACGAGTAGAAATTGAATCATAATAAATATTGTA
CATACAATGTAAACTATAAAGTCCATGGGAAGCGACAAGGATCTCAATATTAAAGCTTCTGCCACCGATT
TAGAGCTGTCGACAGAGATCGATATCTACAAATTAAGGACAGTTGATCAATCTTCGCTATATTACCCTAC
GCATAAACAAGCATACCAACCTGCATCGATCTCTCCCATGGCTACAAAATATTTTGTCAGGTAATATTAG
GTGTCATCTACCGCTAATGAACAACGTATTATGTACTAATTAAATCTAATATTTCATAATGTCTCAAGGT
ATCAAATAATAGAAAAAACTTGCAAAAGCATCAAGTACCCCACCATTAACTAATCCCTGTTTCTTGTCTC
CCTCTCCTTGTTCTTGTTCAAACATAAATTGTAATGAAGCACCCCATGTGCATGCAAGCTTGTTGCTTAT
ATTTCTTCCTGTTGCTGTTAATTTCCCCCCTTCTTATCTTGCATGAGTGGATATTGCATGTCATTCTTCA
TTTGTATGTTCAAGCATCCCATCGATGTAATTATTCTACAACGACTCAGCAACTCAAAAGACCATAAATT
AATCTATAATTAATGGAATGTAATCCCTTAACTTACAGGAAGAGTTTACTGGTTTTTCGAAGTTGATCTC
CTCAACTATAATTAGGTCAGTCGAGTTAATAAACTTGCAGTTTAACTTACATCTATCTATTTATAAAGTA
TTTAACCCCAAAAGATCGATTGGTCTATCAGCATTTAGCTAAGGGCTTGGGAGGGTTCTCAAGTTCAAAT
CCTATCAGTATTAAATTTGTTAAGATCATGACTATAAAATTTAACTCTATATAAAAAAGCTAGTTATAAG
ATGTAAAGATTTAATGTGATGAATGCAAGCTGACTCAGAATACTTTATATTTTAAAATAAATAAAAAAGA
GTATTTTAGCTTTTATCCACAACTTTTTAAATGAAAAACTTGGAAATTGTTTTAATATATACACATCTCA
AAAGAATATTAATTATAAAAACAGATATAGATCATTTATCCAACATGAAATATTTAATAAATGGTAATTA
ATTAAATCACCAAATTAAACTTGATTCAATAAAATATGAAATCACCAAAGAGACATGGCACAAACGCAAT
AAGTCAGTCAAAAAATCAAAGATTCATGAAAATGGAAAATGGCGATGAATATTGTCCACGTTGTGAGAGT
TGTTGAGTATCATCGCTATAGACTAGACAGAGGTGTCGAATCAAGAGGTTGAGATTTTTTATTTTTGAAA
AAAGAAGACAAAAGGGTTAGTCATTGACGGCGTGATTGAAGTACCAGCGAAGCCACGTGAGAAGTTTTGA
GAAGCCAAAGAAACATATTTTGTTACAGATCACAATCAACGTTCGAGTGGGGACCCACTTATAATATTTG
CCACCTTTCATTGAGATTGATTGCCAACTGGCAATTCCCCCACACCTCTCTTCCCTATTTTGTCCTTCCC
ATGCCATGCTTATTTACAAGACCCACCCTTGCTTTCAATCATTCTCGGCCAAACCAACCCTAAATTTGTG
GACACTTATAGCCCCGCGGGGATGACCTTGTGATGCGGGGGGGGGGCAAAGGGCTAGAGAGGGTCATTTG
CATGGTGAGGTTGGCTTGCGTGCATTAATAACAAGTTGGTATGTTTCGGTAATTAAGTTGGAAAACAAGG
GGTAGGTTTGTTGCAAGCATGTCTATATGATGCCATTGGCCTTAAGCATGATGCGAGACAATTGGCTGTA
ACAGTTGCGTTGAGGTGGACACGAGGATAGATAGATAGCAGAGAGACAGAGAGTTTGAAAGTAAGTGAGA
AAGATGCGCACAGATTCTTTTGGCTGATTCATCAACTTTGCTCCTTTCTTCTCTACTTGTATGAATTTAT
GTTTGACTCGTAAATATATAATTCATGATATTTCTTTCATGTAAATCGATGAATATGTTTTTTTTTTTAA
TATTTTTCTTTCTTAATATATTGGATAGAGTTTTTGATCTTCAACCTCTTGATTATTCATACTTGTTTCT
TTTCCGGATATACAGATTTGTTGAGTACTTGAGACAAGAGAGAT
Sequence ID: 146
Sequence Length: 3258
Sequence Type: DNA
Organism: Poplar sp.
CCCGGAGAAGCAATTGCAAGACAATCCATCGGTTTTTGTCTCGTCTTTTTCTCTTTTCCTTGTCACTTCA
AAAGTATATATATATATATATATATACTGGAGAAGCTAGTGATCTGTATCCATATCTATTATCATAAATA
TCCTTTTCAACATGGATTGTTCTTTATATTTAAATGTTTTTTTTATATATATATTTTCTCATGTAAGAAT
TTAACACACCCACGTAAAACTAAGGAAAAATCATGTAAAAAAATAATCAATATGTGTGTTAAACTTTGAA
AGACTAAAATAATTAAGATTTTATTAGACTTTTTATAAAAACCAAGGTCTAATGTGCTCTAGTCAGGTGT
TAGCCAATGGCCCGTCCTTCTTTATATTTAGTTGAATTTTATATATATATATATATATATATAATAGTAA
AACTATTGCATTTCTCAAGAAAAGTTATGGTTATGATAAGTCAAAATGTATATAGCCATGTTTGTAGTGG
ACTTAGAATTAACTAATTATATTATAGCTAATGAGCAGGATGGAGCCAAAAAATAAAATTAAAGAAGGTC
AAGATTTCAATTGTTTTACTATTTGAACTAAAAATATAAATATTGTTAAGGAAAGGAATGCAAAAACAAT
TTCCTTGCAGGGATTGGGCCTCCTGCTAGCCCCCATGTCTCTGCCACTACTAATGAACTTGTAGTTTAAT
TCACAGTAAATTTTTATTTCATTAAAAAAATCATATCATACCCCGTGTAAAAAAAGAGTGAGAAGAGTCT
CTGTATAAAAACAAATATTTAACAAGTTTTTGAGGATGTTTGTGCCGATTTTTATATTTAGGAAAAGAAA
ATATATGAAACTTAAAGAAAAAGAAAAAAGTATTCAAAAATGTATATTTAACAAGTGAAGCTTAGAATAA
TCTTTATTTCTTACTTGAAGTTTTTTTAGGTTATGTTGGGGCAATTTTTATTTTATAAAAATATAATTGA
TAAATATATCTTATTTATTTTTGAAGTTATTTTTGTTTAGTGAGCACTTTAAATTATTGTTTAAATGACC
CTTAACTTAAACCGATTTTAAATAAAATAACCTAAATCTATTAAATAAGGGAATCTTTGATTTAATTTCA
TTATTTTTTCTCTCCTTGCTAGTCACATGCCTAAAGAGAGTGGCTTTCTATCCACTTGTGCAGAGGCGTA
AGGTCCCAAACAGCATCAATCATGATACCCCACCTACCCAACGAGAGGCTTGAAAATGACTCTTTTTTTC
TCAAGAAAGAAAATCTTTTTTTCGTGATGTGATGTTTATTGTTATTTTTTTTATTTAGATATTTTTATAT
TTTTTTTTAAAATAAAACTAATTTCATTTAAAATTCGTAACTGTTCTTTCTGATAAATATATATTTATTA
AGGTTTCGATTTCCTATTTATCATTCAAATAAAGAAAACGTAGTATTTAATTTTCATTAAATAGTTTATT
TATAATGATGACTTTTTATTTCTTTTTTATTGAGTAATAATAGACGTTTAGAAAGGCTAATTATATTTAC
AGTAGAAACATTGAGTATACACAAACTTTTATGGAGACTTCAAAAAGGCAATGAAAGAGGAAGTGTTTTT
CACAGTGGTTTGAAACCCGGCCCGACCCGGGGCTGGAATCGGGTCGGGTTGATAAAAAAATTGCATAAGC
CATGACCCGGTGTGACCCAGCTGACCCTGCGGGTTGACCCGGTGACCCGGTTGACCCAGCAAAACCCGGT
TGCAACCCGTTGATTTTTTTTTTTTTACTAAAACGACGTCATTTTGAATTTTTTTAAAAATAGAAATTGA
CCCGGCCGACCCGGTCAAAACCTGGAACCCGGACCTTGGACCGGGCCGGGTTTAAAAACTTTGGTTTTTC
ACTCTGTGTTTTATATAACGCTATTTTAAACTATTTTAAAGTTAACATATCATTCATCATAAGTACCTTT
TATCTTTTTTTTCTTTCGTTAAAACATCTCACTCTTATTTTTATTTGTAGAATTATTTATATTTTAAAAA
ACCATCCGAGTAATGAATTTCATTTTACATGTTACATGTTAAGCTTTTGTAATTTTTTTTTTTAATGGAA
ACCGAAAATAAAATAAAAAATTCCAACCCATTTTTTAATGGCACAATGAAACATGGTAAAGGGTGAGCAA
AAAAACTGGAAAATCAATTAAACCGAGAAAACCGGAAAAAAATAACCGAAAAAACCGAACCGTGAAAAAA
AACCGATTAAACCGATTAAAATTTTGAAAAAACCAGCTGGTTCGGTTTCGGTTTTATAAGTCTGAAATTG
AAAAAACCGAACCAAATCCAAACCAAAAAAACCGAGCCAAACCGAAAAAAACGAGCCAAACCGAAAAACC
GACCAAAACCGGTTTTTGTCCTAAAAAACCGAGCCGAACCGAAACCGGTCGGTTTGAACCGGTTTCGGTT
TTTTTTTTAAATTTCGGTTTGGTTATTGTTTTTGATAAAAACCGAACCGAACTGAAAATAATCACCCCTA
ATAGTAAATAAAGTTGTTTTTGTATTCATTATTTTTTTTAATTCAATAAAGCATGAACTCAAAGTCACTA
TACTCTAAGCTAGCATTACTCGGTGGTGCATTATTTAGACCTTTTTTTTTTAATTATTTTGGTCATATAT
TTTAGGTCATGTTTTAGTAAATTTAACTTAATTTATTTTGAATTATTATCACTTGAATATCTTATTTTCA
TGTTTGCACGCGGTGTAACAAAGGCCATATCTATATAGTTTGGAAACCAGAAAGAAAAGAAAAACAAGCA
AATTACAACGATAAGTGAGAAATCAGTTAACAAAAGAAAAAAAAGATGGAAGAAATTGTCAAATGGACCG
TTAAAGCGCCTAGAACCAATGCTAGGCTAAAGAAGATTTTTAAGCGTGGGGCTAACATCTTCCCTGAAAA
ATGACAGAAGATTCATGATAATTTTACATATATAGAAAAAGAGAAATGAGGTTGACATGAGAGGAGGAAA
GATGGCCAACCCCTACCTCCATTATCAAGATTCCTTCGGACGTTTCCTCGAGAAGAGTGTGCTTCTCTCA
TGCCTTAAGAGGGTCCCACAATTGCCCTTCCTCTTCATCGCAGCCATTACTGATGCGCTCTCTCCTTCTA
TAAATAGAGCAGGGCTTGAAGATATAACAACTCATTAAGCTACACAGAGAACACATTCATACTCCAAGTC
TTTTAGTTCATCCATTTAAGCTTAATCAATCAATCATC
Sequence ID: 147
Sequence Length: 3079
Sequence Type: DNA
Organism: Poplar sp.
GGAGACAACTACCTAGAATATGACTAGGATGAACATGAAACTAATGGCCTGTGTCGAGAGACTGTGTCTG
GCTATAACAGCAATAACAATGCCTTGGAAGCACTCTTGGACTATACTATCAAATTACATAATCTATTTTA
AGATTTGGTAGTACTGAAAGGTTTAATCTCGATATATAATTAATACCTAACCAAGTGGATCTCACCACAA
AATTGTTGAATTTTTAGTTTTTTTTTTGTATATTTTAATGTGAATTAACATACATCTTATATGCAGGAAT
ATATTAAAATTCTAATGTTTACAATTTACATACATCAAACTCCCAACAAATTATGTCATGTTTAGCTTAA
AAAAAAAAAAAACTCTTCATCTCCATTTTTAATTTATATGTATTAGGATATATTCAATAAAAAAAATATT
TTTATATGGATCTCCATTTCCAATTCTCTTCTCTATCTTCAAGGAACAATTAGGAAAAAAAAAAAATTCC
ACACGGCCTCAATTTCATGATGTAAATCTATGAACCAAGAAAGCACCACATATGGATCCAGGATGGATCA
TTAACCCAAAATAATTTTGTTTTAGTAATTTTTTTTGAAGAAACAATCTCATCTTATAAAAATTAAGTAA
AAAATATTGAAGTAAACGTGGTCAATTCTCGCCTGGATTGGCTGAATCAACTGGATCTTCTGAACTAGAT
AAGATTTAATGACTATAATAATTCCAACCACAAAAAACATGTGCTTAGGCCCTACAGCCTAGCTAGTCAT
CAACTGCTTAGGCTTCTTGTTATTATTTTTCTCTCTTGAGATAAGTGAATAGACTTTTCAATTTATCGTG
TTTTCCAAGTCAATTTGGTGTAGCAGATTCCCTTTTGGAAATCAAATCAATGTGTTGATTATCATCTTCA
TCTTTTTTTAATTTATTTCTTGTTGCTTGTTCCTTTTAGCTACGCAACTATCACATGATGAAATGCATCG
CTTCTCATGGATTGTGTCGGAACCATCCACCCTTCTTTATTAACATGCAAGTATATATAATTCCTACATG
CACCCAAAAAGAAAAAAAGAAAAAAAAAGGGGAAATTTTATCATCTTGAATGTTGTTTTGTTCTCCTTTC
TTTATTTTTTTTTATTGATGTTAACTCGATCAGTCTAAATTTGATTTAGCTAGTTAGTTGAGTTGTAAAT
CTGTATAATCAAGTTAATCAGGTTTTTCATAAATCAATATTCTTTTTAATTTATTTAAAATCAGGTAGGT
TTTTTTTATTTCTCTATTAAATAATTCTTAATTTTTTTTTTTTAGTTTAACGTGGGTGTCCGGGCCAGCT
TGCGCGCACCTCAACTAATCCCACGGGCCCTGAAGTTAACGACCATGTAAGCCTCCAGTGACCATCATAT
GAGCAACCACAAGGCTCGAACCTGAGAACACAAAAATTTTTAATTTTAAATACATGGAAATAGTGCTTCG
AAACAAAGATGCATACTATTTACTATTCATGATTTTTTTTCGTGGATTTAAGAAAATAATCAGTGGATTT
AGTGTTTCCCTAATAAGGACTTTTCAAATATAAAGGACTAAAATGTAAGATCTCTCAAAGTTAGGGATGC
CTCGGCCTCCTCGTCCTTCAACACCATTTTCCGCCCCTGGGGTTGAGAGTTATTTGAAAACAGTTGGAAT
CAAATTGATAAAATGCATATGGTTTAGGATCTAATCAAGAATGAGTAAATAAATTAGGGACTAAAATGAG
AAATCCCCGAGAAAAAAGGCTGTAAAATAGAATCGCTAAAAAACAAAGACCAAAGTCAAGTTTCCACTAG
CCAAATAGGATCACGTTTAATGGCTCACGTTTCAGCACGTGACTGGGACAAGTATGAAAGCAAACAAAAC
ATTATTTTTATTTATTATTATTCTATATTCATTAAAAAAAAAAAAAAGACAGTGAGGATACCGACAACTC
TGGATCCAATGCTTGATTAATTTGATTTATTAATTATTTGAATTTATTAATTATTTGAATAAAGCTTTTC
TTAGGGAATTAATTTGAAAGTTGATTGGTTAGGTTGTATTTCGCATAAAATTAAAATTTAGAAGAGAGTT
TTTACAAAAAAAACAATAGTTTCCAAGATAATTGTTAAAATTGAAAAATTAATTTTTAAGGTTAATTAAT
GTGTTAAAAATGTAAATTAATGTCTCTCAAAGGTAAAATTTCACCTAATTATTTTTAAAATAATTTTGGT
TAATTATCATCCTCTTTTAATAAATTACCCTCTGCTAAATTCTCCCACTTAACATACACTTACCTTTACC
AAACATCTTCATAAAATAATTAATTTTTATTTTTATTTCTGAGAAAAAATGAATATATATAGTTAATGAT
ATAATTCTCGTGTTTTCACATTAATGTAAATAACTAAAATTAAAATTTAATATATTTTTATTAATAAACT
ATTATCATAACTTAAGTTTCAAATAGATTAATGGAATATAAATAAAATCTTGCACTATAATGATGAAGGG
AAAATGACATAAATAATTATGATGTAGATGAATATGTTATCATCACTTCATTAAGTACAAATTTACTAAC
AAAACAAAACTAATTATTTTGTACCGTAGAGATAAGTATTGGCCACTAAATATTATTAAATATTTAAGAG
TTTGATAGCGGTTATATTTTATAGTGTTTTTGTTTAAAAAAATATTAAAAAAATATTTTTTTATTTTTAA
AATTTATAAAAATATAAGATGATATTTAAAAATATGGTTTCAGTTATTTTTTAAAATGTTTTTTATTTAG
AAAAATATTAAATTAATACTTTTTTATTTTTAAAATTTATTTTAATATTAATATATTAAAACGATTCAAA
TCATTAAAAAAAAATTAAAATTTAATAATAAAAAGAAAGTTAAAAATCACTACCGAATATTGCCTAATCA
GTACAAATCTCAAACCCCGAGGTGGACTAAAAAATATATAAATAAAGGGTCCCTCCTTCCCATTCCCTCA
CTGTCAACAACACATTTCCTTCAGGCTTCATAATTCTGCAAAGAGAGAAAAGACAGATATACTCTATCA
Sequence ID: 148
Sequence Length: 2746
Sequence Type: DNA
Organism: Poplar sp.
TTGTGGTTTGAAAAAAATTGTTTTATAAAAAGTATTTTTAGTTGAGGAAGTTGTTTTATAAAAAGTACTTTTAG
TTGAGGTTGATTTGAAAAAATAAATGTTTGGTTAAAACTGTGGTTGAAATTGAGGTTGAAGAAAAAGTAATTTA
ATGTGTTTTGTTAAAAATGCTTTTAAAATTGAGGTTATAAAATAATTTTAAAAAATATATATTAATATTGATGA
TTTTTAATTTAAATATTGTGAATTTAACTATTGTTATTACATCATGAAATAAATCATACTTAATATAAAATATT
TTTTTATTATTCCATGAAAGCATCTACAATTCCATTACGTACGAAATACATCCGATAAAAATTACAGTTTCCAT
AATTATTTTAGTGTATAATAAAATTAGATAAAATATTATCAAGCATTTTTTAAAAAAATATTAAAAAAATTAAC
ACACTTAAAAAAAATTAAATTCGCGGAGTGCAAATGAATAATGCGAGAAAATTGCACTGTTCACTATTTTAAAG
TAAACAGTGCAATAATGTACCAATATATACTGTTTATATTAATTATACTGTTTATTAAACAGTATAATTAACAT
TAACAGTGCAAGAAAAGCAAAAAAAAAATTGCTTGCACAGAAGATAAGGTGAAACATTAAATTTTCATGGGTCC
CATAACCAAAACACAGTTATTAAAAATAATCAAACATCAAATTATTGCGTTTGATAAAAAAAGCAGAAGGTAAC
GAGAGCCAAACACTATCTTAATCTTAATCTTATGTAGTCTGGCCATCCTCTTCTTATCCAAACACGTGTAGCCA
GTCCATTGGAAGCTTGAAAAATGGATAGATGAGAAAATCACGAGGACTTTTTTGGACAAGCACTACTTTTCTCT
CCTCTTTTTTGGTGCATATGTCAGTGGTTGTTGTAGTCATAGACCTTTATGTTGTCGCCTCACCGGAAAGTACA
CAATCTAGACATCGGCAAACACATCCTCTAGGAATAGCCATTTTGCTCTGGGCACTGTGCCCTTTAAGGAGTTC
CTTGCTTTAGGCCCCATTTAGAATTGTAGCTCAAATAAAGCTTAGCAAAAATTTATTTATTTTTATATTTTTAA
ATATATGATAATAGCATAATTATCTTAATACATTTTTAATAAACATTGCTTCTCATCTCTTACCAGCAAGATGG
CACCAAAATCAAGAAGGTAGTACATAGTAAGAGTAATAATAAAGACTGGTCGGTGGGAGACTTGCTCAGGATCC
AGGGCACTTCCGGTATATATTATTAGAATGGCCGGTGGGAGATTGATCAGATACAGTGAATGCATCATAAGAGT
TACAGTGCTAGCTATCTGATCTCTTGAAATCCCATGAACGAGGACCCGAGAGACCTTTTCCTTATCTTGAACGT
GGCAGCCAGAGGGAGTTTCCATGGCGGTGAGCCTGACAGCAGGATAACATTTCTAAGGTGTCGGCAAGCTACTA
TTCACCGCCGACGTGTCAGTGTGTGCACGAAATGGATAAAGAAACTGGTATTTTCAACGGCGGTAAATTAAAGC
ATTCATTAATTCATGCCCATTGCCCAGAAGATAGAAGGAGAGAGGAATAAGGGTACGATAGAGATGGGGCCCTT
TTTCCTGGTCCCACAACATTGTAGATATTTCTTCATCATCCATATCTGATCATTTTTGTCAGCACTGCTCCTCA
ATTCAACATCATCGTTCCACGGTTGTTTTTTTTTCAAGTGACTTTTGTTTCGAAATATCTGGAATTAATATTTT
TTTAATCTTATTATATTAAAATAATATGAAAAAAAATTAATTTTAAAAAATAATTAAAATCCATGGCACGCGTT
TAGTTACAGGCAGAGGGGCAATCCTATTGTAAGAAAAAAAATATCTTTATACTATTTTGGATATTGTACATGCT
TTCTTGTTAAAATATGTTTGGTATAATAGTTTTTATTTTTGTCTGACAACATAGAAAAAACTGTATGATTATTT
AAATAATTTATGTTTTTTTTACCTGACTTATATATAATTACATTTTAAATTTTAATTTTATAAAAATTAAAATA
ATATATTTTTTTATTTATAAAGATTTTTTTTATCCATGAAAACTGATTTTTATTTATTTTACATGCAAAAATTC
ATCTCATTATAATTTAATAAAATATTTTATTTTTAAATTTATATTTTTTAAACCGTAACTAAATTTTTTTAATT
ATATTTTTAAAAGCTAAATACTATTGTAATCTCAAACACACAGTTAACCCATCCACTACTTTTCTATTATAGAT
TCTAATTCTCTCTCTCTCTCTCTCTCTTGAACAAGATATCAACATTACTTAAATCCCATCAAGTGCCTCCTGCG
ACATTTCTTCTTTAAATTATTTGGCATAATAACATGGATATATGTTTATTTAAAATGAATTTGCTTCCTTCCCT
TGTATGGTGCCCTAAACAGGGACCCAACAACTTTCAACATCACCTTTAGCCTTTCGGATTAGATCAAAGGCCTT
TCTAAAGGAAATGATACGTTGGAGTTTATTACATTTATCATCAACACTGTCTGTACACGTAGATGGACATTTTT
GTGATTTCCAGTTATACCATTAGTTTTATATTCTTTTTCTGTCTGCTTCCATTTCCTGTGCTCCACTTCTGAGC
CTATAAATGTGGCACTTGTTTCATTCATCACTTCATATCAGAAGCCATTTCAAAGAGAGAAAAAAGAAGAAAGA
AATACACA
Sequence ID: 149
Sequence Length: 534
Sequence Type: DNA
Organism: Poplar sp.
AAACCTTATCCTTGGTCTCTTGTCAGCGTAAGCATTCAACATAGATGGATGATTATTATTATTTTGTTTT
TAAAGATAGATGATGGATTTTTCTAAAAAAATATCATGGTTCGGTAAAGATTTTGGGATAATAATACATA
TTAAGAAAATATTTATAAAATAACGTAGTTTTATATTTTAGTATATTTTTTTGTTCATAATTTTTATTTA
GAATAATAATTGCATTTTAAAAAAAATTCTAATATATATTATTTAAAATTAATTAACCTAATCAAAAATC
AATTAAAACTTCTATAAGATTTAAAAGTTAGAAAATAAGATTCAAAGTACAAAACTTTTAATTAAAGCTT
GTGTTTAATTACTATTCTTAATAACAATCGTATTCAACTATATTATTTTATTAATACTTTTTTAATATTA
CGAAACTCTAAAATATCTAACACGACATCTATAAAAAAGAAATGAGGGGAAGGGGGTCACAAAGCAAACA
TAGAGAGAGAGAGAGAGAGAGAGAGAGAGAGAGAGCTAAGGACA
Sequence ID: 150
Sequence Length: 3250
Sequence Type: DNA
Organism: Poplar sp.
TCTACATTATACTATACTTTAAAATAAAAACACTTGGCTAATTAAACTAGTCTAATAATTATATGTTTGT
GTCCTTTAAAGATATTGAAAATTTACATTATTATTAATTTTAGAACATATCATATTAATTAAAATACATG
TAAATTTATCTAAATATTCACATTAATAATAATAATAATAATAATAAAAATCATTATACATGTAACTTGG
AGAAAAATTCATTATATTATAAATATAAAGTGTTTTTACTTTAAATAATTTAGCATCATTAGAGCATTAT
TTTTGTTGTTTTTTAATTAAAAAAAACTGTAGGGGCATTGTTGGGTCTGGTTTGACAGCTAGGCCCAACA
CTATTAGGTCTGGCTGAGCCGTCAAATATTTTCAATTTTATTTTTTTATCTCTTTAGATTTTTTCTTTGC
TCTTATTTAATTTTTTTTCATTCGATAAAGAAAAAATAAATTAATAATATTTTTTTTATATTATATAAAA
GTTGAATAATGATAATTTTTTTTATTTAAGATTGAGTTGGGTTGCAATTCTTTTTTTTTAGCGGTTATGC
TGCCACCAGGATTTAATAGTGTTGGGTCCAACTTCCAGTAGGATTTTATAGCATTGAGTTCGGTTGTCTA
GACTCAACAACTTTTAAACTTCTTGATTGTTAGGTCCAATGCTATCGGGTCGTATGATTTGACCACTAAA
TCTAATAATATTGGATCTGACATTACCATCAGACATAAAATACATAATATATTTTATATACACTTCATAT
TTTTTTATATTTTAAAAAAATTATTAATGATCCGTTGATAAGTATGGGAAAATATTCTAGTGTGTATGTA
TATTTATATAACCTGAAAGTTTAAAGGTGTTAAACTGTTTTTATTACTAGTTGGTGGTTTTGATTGGAGA
TCGAGCTGCAGAGATCAGCAATGTAGACTAAGTCCTCGTTTATTGTATGGAAAGTAATTTTTAGAAATTA
TTTTTAAATTGTTTTTGTTTAATAAATAAAAAATATTTTTAAATAGAATATACTTTTCAAAAATTATAAA
AAATTGAAAAATATCTTCAAAGTGCAAATACTGTCTAGATAACCAGTTCCGTCTCAGGTAGACCCCTCAA
GCCACCAAGTGAACAAGAGAAATTGTGAGGGAAACAAGAAATAAAGTTAAACAAGAACTGACATGATTAA
AGTTCAGATTAAAAGATTTTTTATTTGTCGTTTACTTGATCTAATGTGGAAAGTGGAAATGGAGATAGTA
GATAGACTCCCCCGCATTAATGTATCTATCCTCATATAAAGTATGAATAATTAAAATATTTAAAAATATA
TTAAAATAATATTTTTTAAAAATTATTTTTGATATTAAAAAATTAAAACAATTTAAAAATAAAAAAAAAC
ATTAATTTTAAATAAAAAAAACTCAAACACTATTTAAAAAACGTTTTACACGCATCTAAACTTTTTTTAT
CACTTAATTTTTATTTATTTATTTTATTTAAAAAATTTATGAAATTAGATTTTATTTTTATTAATTTTAT
TATCTTTCGATCTTTTTATTTGTTAAATTTGATTTTTATTTTTTATTATTATTTTTTATTTTATTTAAAA
TAATTTATGAAATTGATATTTGTTTTTCAATTTTATCCTCCAACTTTTTTATATATTAGATTTTGTCCTT
ATTTTTTTAATAAAATTCAAAACAAATTAATAAATTATTTTTAGCTCATTTTTTCATGACATAATCAAAT
ATTAAAAAATATTTTTTAATTTTTTTAAATATTAAAAAATAATTAATTTTTTATAAAAACTACTTACGAA
AAGTAAATTATTCTCCAGCAAACAAGTTCGACAAGCAAACGGTATCCGAGAAAACCTCTGTAGTTTTGTC
TTGGCTTCTCTTAACATTCTTTCTACCCCTTCTACAACGATATTTTCATAATTAAACTAAGAAATAAGTG
AAGACTTCACTCCCTTTTCATGACATGTATGGGGTTTTTTTTTTTTTTTTGCATGAGTGTGTTCTAGTTT
AAGATATATTTTTTGCACCCCTGGCCAATACGCCCACTCCCCGATCAGTTGATATCATATGCATTATTGA
ATTATATTGCAATATCTTTAAACTACGCTAGAAATTAAATAATTGACTTTTTGATTAATATCAATATCAA
GAGGCCATGAGTGGATGTGAGTAAAATCAGGAGAAAGCGATGCAGACAAATTGCGAATCCCTCAAAGTTC
AAAAAAATCCATGTTGGTTTCAAAAAAGTCCGAATTATTTTTATAAATCATCATCCTTATAATAGAAGAA
CTTACATTTTAGTAATATATGCCGTAATTGTTTTCTATACATCACATTCCTTATACTAGAAAAACTAACA
ATTTAGCACATAATACATAACATATTAAATTTTTTTAATATGTTTGAATACTTTTTTATTTCAACGTATT
CATTCCCTCATCCCCAAAGTAAATTATTTTAGTTTCAAGAATAATATAAAAGTATTCTTATAAATCTGGT
TCATCTGCGTTCTTAACATGATAGCCGGTTAGAAATTCAAATTTATTCTCTGTAAGTGAATGAGTAAATT
AATTATAAGGTAAAAGTGCCTCAGTGAATGACTCCATATCCCTTTTTTTTTCATGCTTTCAAGAAGAGAC
ATCTGGTTCTGGTATTGCCACGACGAAGTATTACTGCTGAATGGCAGAAGAAAACAAACTCGAGTCTTGA
TGTAGATAACGCTATCGGTGAGAAATCTTGCTGTGGATAAAGTTGTCGGTGAGAAATCTCGAAGTAGATA
AAGTTGTCGGTAGGGTTTTCTGTGTGCGATTGAAAATATTTTTTAATCACTTCGAAGTGATTGTTCGGCA
ACAAATGTGCATTGTAATCGTTTTTGCTATTACAGTTTATTTTTTAAAAAATATTTTAGTTTTATTTTTA
GTTAAAAAAGATAGATTTTCACATATAGAATAAATTATAGACAATTTTATACGTGGTTCAATATAAAAAT
AAAAAACTATTTAGTTTACCAGATGTTTTTTTTATTAGATATAAAGTAGAAATTATGGCAATGGCAAACG
GTATGTAAAAATCCCTGGATTTTTACCATTGATCACGTGACATGCATCTTGTTGAGTAGTATAAATACTC
TTGAGGGAACCCAATCAATTTCAAGTTGAAGAAGTGCTAGATACTAGTAGTAGTTGCAGACAAAGAGCTA
GTGTTAGACAGGTTCTTGTTAGACAGAGAG
Sequence ID: 151
Sequence Length: 3088
Sequence Type: DNA
Organism: Poplar sp.
ATGGTCAAATTTGAATGAATATAAAGGATACATGGACCATGTTCAAATCAAACAAACATATGGTGGGTAT
TTAAGAAGAAAACAAAATAACAATAACAATAACAATAACTAGTCAGACTTATATAATGAACAATAACAAT
AATACTCATTATTTCACACCAACACACACACACACACACACACACATAATGTATAATAGAAATAATTTCA
CTCACCCGAAAAATAGAGAAAAAAGATAAACGAATGCAAAACCATAGATTATTGAGGCTCGCTAGGAGAA
ACATCAGCAAAACTTATAACGGATATGGAAAACACTAAAAAACAACTCAAAGCAACATAACACAAAAGAT
TAAAACCGTAAACTTAGAGACCAAATTGTAATAATAAAGTCGAACTGAATTCACTTGATCGATTCCGAAC
ATAACTCAAACTGATCGAACCTAGCAGACCTGATTAACTGTCATTACAGGGTTAATCAGCTATAACATGT
GAATCGGTCGACTGGTTGGTCAAGATTTCCAGAATCCTGACCTGCTAACCAAAAATCTGCAGAACCTAAA
AGTAATAGACATTCTATTCTTCAATAAACTCATATTCCAGACATCAACCCAATTCATTTAACTAACACCC
TAATACATAGATTAAATCCCTTAAATCATCCATATATCAAATCAATATATATTAAACATCTTAAAACCCT
AAAACACTTCCTTAATTCATGAACAATCAAATCCCCAATCAAAAACTAAAAAGAAGGTAGTCATAATACT
TACTTACTTCCCCAACCCTTCTTAAAATCCAAAAGCTTAAGTTTAAAGTGAGTTGAATCCCTTGGTATTT
TGAGAGAGAAGTCACGGTAAGAGAAGGAAAAAAAAAAACCTCTGGTCACATACCTTTATATATATATATA
TATATATATATATATATATATATATATAGGTTTGGTTTGGTTTTTGGGCTGACTTGGGCTTAGGCTTGTG
TTTGGTCTAAGAATTTGGGTCTTACATTACAAATGTTAACCTTAAGATTTAAAGAGATAAGAATGTTGGA
AGATGAATCTGTTAATGAGTTTTATGTAAAAATTAAAGATATTATAAATTTCAAATTCAATCTAAGAGAA
AAAATAGAGCAGTTGTAAATTGTAAGGAAGATTCTAAGATCCCTACCTGAAAGATTTCGACCAAAGAAAA
GCCATAAAAGAAAGGAAGGATTTGAACACTATATGTGTAGAAGAATTCGTAGATTCCTTATAAACTTATG
AATCTACATTGCCTCATAAAAAAAAGATAAGTCCATTGCATTTAAATCTATTAAGAAATAATATGATTAT
TCCTTTGATGATGATTTTAATAGTGAAGATATTGCTCTCACAGCTACGAAATTCAGAAAATTAAGTTTAA
GAAAAAGAACAATGGTAATGATTAAAAAACAATTTCCAAAAGAAATGAGTTAGAAAATGGAAGCAAATGT
AAAATTAAATCTAAGAAAAGAGTTAAGTGTTTTGAACAATTGAACAATTTTATAATTGAACAATTGCAAG
TATTATCAAACAAGTATAATAATTTTACTATTAAGTGGGAAAAAATATTGATGTTGTTGACAAGTTGCTA
ATGAATATGCAACAAGATATTATACAGGTTAATTATATACTTGTTTAACTCGACTTGGATGTCGAACAAG
TGCATTAGTTTGTTGTTGGTGAAGATGAGAACTAGGATTAGTGTCAAACCAAGAGGGGACAACGATGCAT
GCAAGGGATGCAAATGACAAGCAAAAGGAGAAAACAAGTAGCCAACCAGCCAACTGTCATAATTGTTTTT
GCTTTACTAGAATTTTGCTGATTTTTAATTATAAGTAATGAATATTAAATATTCTGGTCTGTAATAATTA
TGATATTGTTTTTTGTAGAATAAGAACCTCTATGTTGAATATTTGATGCACTTTGAACTTGATATATATA
ATTGTATAACAATATCTTATATATGATAGTTTTTTAATATATTATGAATTGTTATGATACAATATGTTGC
ATATAGTATTTTTTAAATATGTTATTAACTGTTTACATGAAATATATTAAGATCGATTAATTAGGATGAT
GTTGCATAAATATTATTATATTAATTTTTCTCATGCTACTTGTTTCAAAATTCTCAAAGTAAATGACTTA
TTAATATCCTTCTCCTTACTTTTCTTCCACTTACTTTCTCTTAGTACATTCACGCTATTACTAATTATTT
ATATATATCCGTATTAATATACACCTAAAATAATTACTTGAGAGCAAATCTTTTGCTTAACCAACACAAC
TTCCAATCATTTTTCCAATGAATACACTTTTTTAAAATGATTTTTTCAATTGAGAATGGATCTTTCACAT
TATTAACTTCAAGGTTGGAGCATGTGTATTTATAATGTATTTTTTAATTAAAAATATATTTAAATGATTA
TTTTTTAGATTTTTTTTAATTTTTTATTGAAAAATATTAATTTAATATTTTTTAAATAAAAATTAATTTA
AAAAATATTTCAAACTACAAAATCAACACGCTTTGACTATATGATGATTAACTCTTGAGCAAGCATTTCA
CACATTCGGGATGGAGTAACAATAAAATATTGTAAATCATCCTAAGATTCAAACGTTGAACAATTCAACA
ACATGAGGAACAAAAAATTATGTACAAGTTGGAGTGAAACCTCCAAGATTGCATGGCAAAATTATACACT
ATGTGTTGTTATCTTCAAATATCAAAAGCAACGCGTAAGTGAGAATGAAGCACTTGATGAATCGACACAT
AAAAATATTATTTTCATAAAAGATAATACTCTGCACGTTAGAAACAAAGCAGCAGGTACTATGGTGTTAA
AAAAATAACACAAGTTGGAAGAAAATGAAAAAGGAAAAATACACGTTGCCAACTATTAAGTATTGTATAA
AGTTTCAAGCTGACGCCCCAGACCTCTATATATAGCCCTGGTTCGTTCCTTTCCAAAGCACCATCCCTAG
AACTCACTTCTTCCTTCTTTCTATCTTGTTTTCGACTGCAAAGCTCTTCCCTTTCTATCTTGTTTTGCTT
ACTTTTCA
Sequence ID: 152
Sequence Length: 3102
Sequence Type: DNA
Organism: Poplar sp.
TTAAAAAATTATTAAATCATTCTAACCATATATGTGTGTGTGGTTAATTATTTTAAATAATTTGAAAATA
GGAGAAATAGCTATTTTATTTTAATTTTTTAATTAACAAAAAATCATATGATTAATCTCGAATATCAAAC
ATTTAAGTCTGTTCTTTCTATCAAGTATCAGTGAAGAATATTTAACATATAATTGAATAACATAGATGCA
ATAATAAAAGAAATAGTAATGAAAAAAATATAAGCATCTCAAAAGCTATACCAAGATATCGGAGTGTTTT
TGCACGCCTTTTCAAAGAGAGGTAGGCAGTAGGCCAGACTAGGCAGGTTTACAAAAATAAAAAATAAAAA
ATTGGGTCCTCCTCCAACAAATGGGCGGCCGCTGAGAATTTGGAGTCGGACCAATGACTCCTACCTCCAC
CTTTAATAATTGCTAGATTTCCAGGTCTTCACAGGCACAAAGTTTTAGCAGTGTATTATTTTAATTTTGA
CTTTGATAATAGTTTCTGCTTTGACCACCTAACTTCTCCATGGCTAGCCAACCATCTTTAAATTTTGCTT
TTATTCAACGCATGCTGCCCAGAAATTTAAAAATGCTCCTTGTTTGACCCTTTTTTGCATTACTCATGTT
ATTTAATAATTGTATTGTGTTTGTTATGTCATGTTATGATGAGTTGAAAAATTAAACATAATTAATTGTT
TGATAATGAACTTCTATGTTATATTGTTAATAAAATTATTTAAATTAAAGAATTTATTTAATGAATTAAA
TATTTTACTAAAAATGCCAGATTGGACCAAGAATTGTTATATACTTTATACCCCGTAAAAATGAAAAGAC
ATTAAAAAAAAAAAAAAAAACAAGAATGGCCGAGGACGTCATCACATAAGTTTAATATATTATATACGAT
GGTGCATCAAGATACATATAAAAAAGACCCAAATAGCCTAACCAAGGATACATGTAGCCCAGCCAAGGCT
CTTTGTTAATGCCTGAACCGAGGCTGCTTGTGGATGGCCTAACCGAGGCTATTGGCCTAATCGAAAGAGA
GAAGGAGAGGAAAAAAATGAGGCAGTCTACATATCCAATCGAACCCATGAGTCCAGCCGAGACCGAGAGA
GGGAGGAAAAAAGCTGACCCAATCAACCCCTCTGTAGCCTTCAAGATTAAGATATAAGATAAAAAATAAA
TTATAAAACTTGAATTAGCCCAACTGAATCAGCCCAACCTTGGCTTAATTCAACTTAACTCGAATTATAA
GATTAAGATATTTTAATAAAAAATAAATTATAATAAATTATAAAACTTGATCTCTAATAAATTTAATATT
AAAAGATAAATTAAAAAAAAAACTAAATTAAAAAAATATATCAAGTTATCAAATTAATAACTTGAATTAT
AAGATTACATGATTCTATCCAAAACAAATTAAAATAAAATATAATGCAAAACATAACTCTTGCTTTATCC
ATTACCATGACATCATATGTGACAATTCTCCATTATCTAGTAAATCTAATAATGAATTATAAATTTTTAA
AAAACTAAATTATAAAAAATAAAAAAAATTAATAAATTCAATATTTCAACAAACCTTGTTTTATGAGGAT
ATATATATATATTTGTTAAATCAAACATGTACAAATTCATAAAACAAAGGACATAATCAATAAGAGCTAG
CAGGGAAGAAACGGAGAGGAGTTGTGGTGGTAGCATTGAGTGAGAAAAAAATAATAATGGTCTGTTATGG
TATGAGCCCACATGCTCTTTACAGATGGCCATGAGAAGCTTCTATTGGCCAATTGAAAGTTTGAAACATC
TAGACCATTTCCTTTGTTTTCCCCTGAATATGTAGCATTGGGTTTCATAAATACACTGGACCGGACCATC
AAAGCCTTGTCTGGTGGACTAAATTGCATCATTTAAACATTTAAAGGGCCTGATGCATAATTTTGACTGT
GACACCTAACAGGCCTACACGGTCCAAACTCGAGGGACCTGAACATCTATGGGGCCTTCAGCTAGTTTAG
GGTTTCATTTTCGATAATGGAAGCTCCATCCCTTTTCTAATAATTCAAGTTTAAAGTTTCTCCTAAAAGC
TCCACTGTGCAATTGAAGATTGAGTGTAAAAGACGCTCCAAGAGTCATTTTCGATCACATTGACATGCCA
ATCGAAAGTGTTCTTTTTTATTATTATTATTAATGTGAATGTCTGGACTAATTTGCGCGTATTTTGATTA
ATTCTACGAGCCATGAAGTTAACGACCATGTAAACTTCCAATGACCCTGAACTTTATGAGATTCGAATTG
GTAACTTCTAGAAAACAAACTTAAAACCTGACTAGATGAGTTACACTCCTCGAGACTGTGTTCTTCTAAG
CAGTGGTGACCATGCCAAAAAAATCAACCCATGCATGCATTCTGGTATCAATTTCTTAGAAAAAATTGAT
TTTGAAAAAAGAAGGTTACACATAATTTAGGGGCATGGTTCACTTTCATTCTTTTTAATGCACTTGATGA
CATATTTTCAAGCTATTAAAATTTTAAAAATAACCGCTATCTTCCTTTCCGGCACCGTCCGAGCCCACAA
CACTACAATTCGTTGCCTGCCACTTCATCCAAATGTGATGTGAACTTAAATGAGTAGAGCCCATATGTAT
GAGCTTAACTAAGATCATCTGGTCAATAGCCGAGCACCACAAGGGTGGTCAGGTGCATCTTAAAGGGGCT
TGAAAGAGAGAGTAGAAGCCGAAGCTCGACTCGGCGATAACCTCAAATGAACTAGTATCACACATCCATG
TCCTCCCGAGGATGAGATAAGGTCCTGGAGCTTCTTCCACGTGGCAAATCCATTGTGGTGCCTAACGATA
AGGCTAGCATTCCAAGAATTTCCACCCTCATGTGGATAGAATGGTGTCATGTCATCACTTATCATATCCA
ACGGTCACATTTTGCTCATTATCAACCACGATAGCTTGTGTCCATAGGATATGAGATAGACAAGTTGAGG
CATTATATATACGGTGTGGCAGGAGTACTTCAATCTTACAATACTAGCAAAACCACATATAGAGGGTGTA
ATAGCTAAGTAGCCTGTAAGAG
Sequence ID: 153
Sequence Length: 3009
Sequence Type: DNA
Organism: Poplar sp.
ATTAAGATTTTCAACCAATTATTGCCATTCAACTTTCAACACCCCACCACAAAAAAAAATAAAATAATAA
TAATAATAATAATAAGACTCTAATTAATATAATTAATATTGTGAGGTGACCTTAACTGGTTTTTCTATCT
TTGATTCCTAAAGATCATCAGTTCGAGTCTCACAAACCTCAGGATCATTGGAGGTTTACATTGTTGTTAA
TTTCAGGGTCCGTGAAGATTAGTCAAGGTGAGCGCAAGCTAACCCGTACATCCACGTTAATAATAATAAT
AAAAAACTTTAATTACCCCACGCGCATATATATATTCAATTTGTGGTTTCAAATAAATTACAAGAACCAA
ACATTTTCGTGATTTTCTTTTCTTTTCAAATAAATTATAAGAGAAAATTAATGCTAACTATTAGTGTAAT
TTAATTTAATTAAAGTCCATATGGGAAAAGGGGCAACCCATAGTCTAGAAAAAGGTGAAATTAGAGAGGT
GGCATCCATGCAAGAAAAGGTCCAAGAAAGCATGAAGAGGCGGCAGTGCGTTCAGACATGTAAAAGGGGA
AGAATATGAGACCACTCGCCACATATACATCCCCCTCGAAAACGCCGTTTCAAGCTCCCGGTTTTCAGGA
AGTCTTTTTCGATGGTTCCATCCCATTTTCTGAGCTAAAAACAGGTGACTGGTTTCTGGTTTTTTGTGTA
TGAACATTGGCGATTAATATCTTAATCATGATTAAGAGAACACTTATAATTATAATCAAGGGACAGCATT
AACTAATTTAGTTGATCTCAATTACATCACGTAATCTTAGGACACCCAACGTGGATTAAATTATGTATGT
AACTTGGTAGAGATTTGCACTCGAACCCTAGATGGTCGGCAGAATGTACTTTTTTTTTTGTGTACAACTA
AGAAGGTTCTCTTTTTCTGAGTTTGATTGCTTTTATTTTTAAATATATTTTTTTTGAAAAAAAGTTCAAT
TAATTTTTTAGTGTTTTTTTACCTCCAATGTACTATGTTAAAAATAAAACACACACACACACACACATAT
ATATATATATATATATATATAAAATATTTGTTTTTGGCTATTAAAGCCATGGTGAGTCTGTGATACAAAT
TTTAATCAAATTGCAAATTCAAGAACCTTAAATTTAGAGTCACTCTCGCTCATTCAAATAAATAAGAGAT
TTTAGGAGGGAATCATATCAGCATCCAACCTATCCATCAATTTTAATCGGATCCCAGAACTACAAATTAA
TTTTCCCAATTGAATTCCTAAAGGATTGGACATTTCTCAATTGAGTATTCCAACAACTATATTTATTGGC
ATTTTCCGTTATTTCATGCAATAAATAGCATGAAACTATTTTGAAAACAATTTGACATTTTTCTATTAAA
AAGCTACAATGATGTCAAATGAAACAAGAATTTCCTAACACGTGAGAATTTTTTTTATAAATTTAATCAT
TAGAAGTTCATAACACATTGGAGTCGAAAACAAAATTCTTACAAAATCTTCACCATCTAAGTTGTCCATA
GCAAATTACATTTTTTACAAGAGCGATAGCGTTTGAAAACGCGGGATAACTCGCTTTCTAAAAAGTTTTA
ATTTTTGTTTTTTGCTAAAAATTAAATTTTTTTTTATGTTTTGAATTGTTTTGATACGCTGATCTTAAAA
ATAATTTTTTTAAAATAAAAAAAATTATTTTGATGTATTTCAACATGAAAAATACTTTAAAAAGTAATCA
CAATATTCTCTCAAACAGGTTCAAAATAAAAAAAAAAAAAAAGAAGAAGAAGAGGGAGTTGCTGGGCTGT
TTAGTAATGTGTAACGGAGGTGAGATTATAATGGGCGGAAATGGTTTTAGCCCGACTTCTCTATAGAAAT
AGCCATGGGCCCATGGCATTCTTCTTTGGGCTAATTTAAATTGCTAGACCATTTTCTTTTTTTTTCCCTA
AGAAAGTACTGGACCAGACCATTAAGAACCCTGTCCACTCCTTGTTTACTTGGCCATTTTCAATGCATAC
TGAGCTGGATTTGATCGTTAGAGGACCTAAAGCATTGCTTACTTGTGACCACGTATTGCTTTAAATTTTG
AGGTTTTGAGGGTGTTGTTTTTTAAATTATTATTTTTTATTTAGAGTTGTATTAAAATAATATTTTATTT
ATTTATTAAGATTTATTCTTGACATCAAAATAATAATAAAAAATATAAAAAAATAATTTTAAATAAAAAA
AATTAAATTTTAAATAAAAAAAATTAAATTTTAAATGATCTTTATTTGGATCGCAATTCCAAACCAATAC
TAAAAACCAGATTGTTGAGCTTTCATGAATGTTTTGGTTTTACTTTCCATAATGAAATTGCTCCAATTAA
AAAAAAAGAAGGTAAAATCTAGTGACTCCAGCCAAACCTAATTGGTTAACTCTAAATTTTTTTATTTTAA
ATATGAAACAATATTATTTTTTAAAAATCTTATCGACCCAAATTGACATTAACTTTTTTTTTATATATAT
ATATATATGAAGTAAACTAAGCTCTAAAATCACTGTGTCAATGGCAAGCTGCCAAGAACCACAGAAGTGG
TAATAAATATCTTAAATTAAGGGAGCTGAAAGAGAGAGTGGAATTAATCGAAAGGCTGCCTCAGCAAGAA
CCATAAAGCCATAAATTAACTAGCATCACACATCCATGACGTCCTCCATGGGATGAGATAAGGTCATCGA
GCTTCTTCCACGTGGAAACCTCTTTGTGGTGCTAACGATGAGGCTAGCATCAAAAGAATTCCCACCTTCA
TGTGGACAGAATAGTGTAATGTCATCACTTATCGTATCGAACGGTCAAATTCCGCTCATTATCAACTCCA
CTGATTTGTGACCGTAGGATACGAGGGGGACAAGTTGAAGCACTATATATAGGAAGTGACAGTAAAGCGA
GTTCAGAATATCACATTTATAGGGAGCACTGCATAGCTTACAAGCTTGTAAGAGATGGCTTCCTCCTCT
Sequence ID: 154
Sequence Length: 3201
Sequence Type: DNA
Organism: Poplar sp.
TTTAATTGCATTTTCAAGAAGACAAAATATAATTGATCTCAAATTAAAACAGACAATAAAAAATAAATTT
TAAATATGTAACTCTATCTTTTTATCAATATCAACTACCATTATTAATTCCTCTCAATTTTATCAAAGAT
AACAACATAAATAAAAATTGTCTTCCTTAACCCATATTAAAAACATAAAATAATAAAGAAATAAAACTCA
TCACAAATATTAAATTAACAACAAAAGGTCTAAAAAATCAAGAGATATAAAAAAGAAAATAGAAAAAGAT
TTTAAACAAACATACCTTTTAATTTCAGCTAATTAACACAACCGATGTTAATAATTAACCGGAGAATAAT
TGATTCTTGTTTTTTTAAATTTGAGAGAAGGGTGAGACACAATACAAAAATTTAAAACTACAAGTTTTTT
CTTCATGTATTTATAGAAAATTTCTTCTATATTTTTTGTTTTAATAAAAAAGTTGTTGGCCAACGGCTAT
AAAAATAGTCATTGGCCAACAACTATCATTTTGGCCATTTCTATAGTTAAATGTAGAAATGACTGTGAAG
AAAACAAATAGCCATTTTGGCTATTTGTTTAGCTCCTCCGTTGGAATGCAAACAGTGAAGAAATAAAAAG
CGAATGACGTTTGGCTTCTCATTTAGCTGCTCGGTTGGAGCTGCTCTTAGCTTGATCGAATTATTCAGAA
TTCAATAAATTTTTAAACACATATTTTTTTAAAAAAAAAACAGTTATAAACTGATATATCAACACATTCT
CATAGAATTACTCCATGATAAAATTTCCACCGTCGCCATTATTGACAACCTCGCCCAGGGGCTAAAGATA
GCAGACATGCTGTTTTTTTGAGATGATTTGAAAGAAGGTGAGAGAATGCAAATGGTTGTTGTTATTAATG
GGTTCTTGGTCTGTGTATATTGGGGTCCTTGGAGTTTCTGTGGAGTTAGTTTCCAATGTTTTTGGGTTTT
CAAGCTTTAATCACATTTTGGATACTAATCTCATCCTCTTTCGATGAGAGGATTCAAACTGTAATGAGAT
GTTTTAAGCACAAGACGATAAATTTAGAAATTCAAAAGAATTATGAAAGATCATACACTCATCACGACTT
GGTAGTACACATGTCACCTTCTGGATACTAATTTACATCACCACACACCACTCAAATCACACACACACAA
ATATATCATTCTAGTAAAACCAAGAATCTCTCACTTTCCGGGGGCGAAGTTTGTGGCATATGCCCAGGCG
TTGTTGTTAACTGGGTCAGAAAGGTGGTCAGCAAGGTTCTCCAGTGGTCCCTTTCCGGTCACAATGGCCT
GGACAAAGAATCCGAACATTGAGAACATAGCCAACCTCCCATTCTTGAGTTCCTTCACCTTCAACTCAGC
GAATGCCTCTGGATCATCAGCCAAGCCCAGTGGGTCGAAGCTTCCACCTGGGTAGATTGGGTCAGTTACC
TCACCGAGTGGCCCGCCAGCAATTCTGTAACCCTCAACGGCACCCATCAAGACCACCTGTGTAGCCCAGA
TGGCCAAGATGCTTTGTGCGTGGATCAAGCTTGGGTTGCCCAAGTAGTCAAGTCCACCCTCGCTGAAGAT
CTGGGCTCCAGCCTTGAACCATACAGCCTCGCCGAACTTGACACCGTTGCGGGACAAGAGCTCGGGGAAG
ACGCATCCAAGAGCTCCAAGCATGGCCCACCTGGAATGGATGACTTCAAGCTCACGGTTCTTGGCAAAGG
TCTCTGGGTCAGCAGAGAGGCCAGCAGTGTCCCAGCCGTAGTCACCAGGGAACTCACCAGTCAAGTAGGA
TGGGGGCTCACCAGAGAACGGGCCCAAGTATTTAACACGGTCTGGTCCGTACCATGGGCTCCCGGAGGGA
ACAGGCTTGGTGGTTTTCCTCATGGAGACACGGCCATTGCCCATGATCTCAGAGGAGGAGGGGTTGAGCT
TCACCGCCTTTCCGGCTAGCGAAGGGGAGGAGAGGGCCATTGTTGCTGCTGCCATTTTCGAGCTGATGAG
TAAACGTGTATGTATTTGTGATTTACGATTGAAGAGATGATGTTTGGTGGCCGTGGGTTGGAGTTGACGG
TCTTCTTTTTATTTGATCTTGTGGAGATGGTCCTTATCTTAGGATTTCTATGACTGCACTCTTATTGGTT
GGTCACGTTTTCGAAGATCGATTTTGAATGTCGACCTGGCACTCTCTACACGACCTATCATCTCCACGTC
ATTTTGGCTGCCGAAAAACGTATCCGAAATCTGGAGCAATGTTTCAAGATGTGTTTGTTCCAATAGAAAT
TTGCCACATATGCTTTTGGTCATCTCACAATCTATCTCTATTAGCTTGGAGGCAGCCATGAAATCTAACG
TCATGTTGTCTAATGTGCTCTACATTATGTTTATAGCAGTTTTACCTCAAATTAGTGAGAACAGTTAGAT
TTATTGTTTCCTTCTGGTGGACAAGAGTTAAAAAGCACAAGACATGGTTGGTGTAAATCTTGCATCAATT
TATCATATTGATTACAATCTGACAGCAATGCCAATTACCAGTTTTAGCAATCTCCACCAGAAAAAATATA
GGCCTCCAAGAAATTCTGGAAGAAGCATGGCTTTAATGCTTCTTGACATGAGAAAAGGATTGTGTTTTGA
ATGGAAAACTGGCTGCCATTATAGCTAGAAAACACTGATTGAATCCACCCCAGATGGGGAGTATCATTAA
TTTGGTTCAAGAAATACAATTAATGAAGACGTTTTCTTAAGAAGAAGAAAAACTTGAAGTCATTTGATCG
AGATATTCCTGGCTAGACAAGCCACAACCTCTCATTATTTTCATTTCATCAGATATTTTGTGAAGCCATT
CTACCAGCCACTCAATTCTGATAAAATGTATACGTGGAATACTTACAACTTCAGAAATGAATCTAATAAC
ATGGTGATTTAGAGGGTGCCAAGTAAGATAATAAGTATAGAGCCCAAAATCTAGGGCCTCCAATGAGATG
AGAGATTCAGATACCCGAAGATAAGAGCTTCTGCTCTATAGGTGTAGCCCCGTGTAATCCAAGAATTTAG
ATACCACAGAGATAAAGGAACTCAAACGCATAGCTTCCTACAAATACCCCAGCTAGCCAATACTCTCCAC
CACTAAACCCAAAGCTCAAAAAAAATCTTAACTGCACCCGCTCATCCAGCA
Sequence ID: 155
Sequence Length: 3226
Sequence Type: DNA
Organism: Poplar sp.
TTGTTGAGGTGCTGCCATTAGCTTATACTAAACTGTATCACAGGGTTCATTGTTAATGCTACGCTCAGAT
TCCGATTCAAGGAGGGTCTACTGGGATCCTTATACTAATCCCTTGATTGCATAGCAAGAACCACAATCCC
TTATTTACTGAAATGGGTGATTCTGGCCAGAACCAAAATAGCCTGTTGAATTTTGAGCCCTTTTTTCCAA
TTTCATTTGTAGAGTGCTGGTCTTCAAATGACATCTAATCTTAATTTGTATCATGGATTTTATAGAAAAT
ACAAGGGATGCTTCTATTCTGAAAGAATCTTTGCCTGAAGGTGCAGCTTGGGATGATAGAAGCCATTAAA
TTTACCACTGTCATCAACAACTTTTGATTTTGTTATATCATAAAGAAAACTAGTTGATTATATGCAAACT
CCTGGGCTGCTCAAACTACTCTACGATGAACTTGCATGTGTTTTTATTTATTTATTTAACTTTTGTATGA
GCTCCAGCAGACCTGACCTTTCATTACATGCAGGAAGAAATTCTGAAAGTACTTCCTGCCAATGAAGCAA
ATCAATACGATGGACAGAGGCTCTTCCGTCCTGAAGAACTCATAGAGCTGTGCTTTAAAGTTCAAAACCC
AGAGCTTGCATTGCGGGGCTTTGATGTGTTTGCATGGACTAGCTCCTCATTTCGTAGAAGTCACAGAAAC
CTTCTGGAGGAGTGCTGGAAGAATGCTGCTGATCAGGATGACTGGGGTCAACTCCATCAAGCATCTAAAG
ATGAAGGGTGGAGTGATGAGGAAATCTTGCAGCAACTGAGAGATACAGTGCTCTTCCAGGCATCAAGCAG
TTGTTATGGACCCAACGCAGAAATCATCGATGAAGGTTTTGATGCTGTCTTGCCATTGAGAAAAGAAAAC
TCTGGAGTTTCAGCATTGGAAGATTTGGATTTCTCTGTAGAAGCAATACTGATGCAGCACAAGGATTACC
CTGATGCAGGAAAGCTAATGCTGACAGCAATCATGTTAGGAAGTGTACATGACAATTCAAAAGTAGAAGA
GAATCCTTCTTCAATGGAGTAACTGACATTGTTGCAGTTCATTCCTATGGATTTTTGTAGATTGAGTAAT
AGAATCTGCAAAACATTATTAATATTCTAGTCTGAAGTTCTGCCATGCTTTGTTCAATCCAATTCAGCGC
TGCATATTTATTATGAAGTTTGAGATTGTGGTTGCGCTTGTTTTTTAAAATATTTTTTATTCAAAAATAT
ATAAAAATATATATTTTTTATTTTTTTAAAATTATTTCTAACATCATATTATTAAAATATTTGTATTAAA
CAAGAAAAATATTTTGAAACCTGTTTACATGCAAAAGAAAGAGATCAAAATTGGTGATGACATATCCATA
GGCATGGATTCAAGCGGTTATATATGAGTTTTCAGTTCTGCTATTGTTACCGTCCTGGGTTTTTGTTAGC
AAACTTAGCATAAACAAATAATAATAATAATAATTATTGTATGAACAAGGACATAAAATAAAATCAGTTG
GATCAGATTCTAACCATTAGTTACCATTTTTTAATTTGCCTATGAAACCTTGCCTCCAAGCATATATTGT
TGGCTTGCCTATGCTGAGAGTCATATGACTCATATCTATTTAAAAAAAAAACAAAAAAACTTAAAAAGAG
ATATTAGTCATAACTAATATTTTTCTAGTAAAAAAAAATTAAATCGTGTGAGAGTTAATCTGAGATAACT
TGGTCAACTCAATGAGTTCAAACGACTGGTAAAAACATAATTTGACTAAAACATATTAAGACGACAACAT
ATAAAGTTAACTTTTTATGTTAAAAAACATATATCGACTTGAGTTAACATGAGTTAACTTTTTAAATTTA
TGACCCGGGTCTTGAGATTATGATAACCTTATATAAAGTAAATCAAAATAAATTATGAATTTCAATTTCC
AATGTTGAAGAATGAATCTTAAAAAAAAAAACTAAAAAAAAAGGATGAAAAAACAACCCAAGTTAAACCG
AGTTAATCTGTCTAATCCATGATCCGGGTCATGAGATCAGGGTAACTCCATAAAAAGTAAATTCAAATAC
ATTATGAAGCCTAATTCCCAAACAACCCAATGATGAAATTGAAAGAAAATTCAATTTTAAAAAAGAACAC
AATAAACTCGAGTAAACCCACCAAACTCATGACCCGTGTCATAATAACAGGATAACATCATAGAAAGTAA
ACTAAAAAAATAACCCGAGTTAACACGGGTTAACTTTATAAGCTTGTGATTTGGGTCATGAGAAAGATAA
TCATATAGAAAGCAAATCGAACACATCATGAAGCCTAATTCTCAATAAACTCATTGTTGAATGATGAAAT
TAGAAAAAAAATCAATATAAAAAAAGATACAATAAAATGACCTGAGTTTACTTGAGTTAATTTGCAAGAT
TCATGAGACAAGGATAACCTCATAGAAAACAAATCGAAGCAAATGACAAATCCTAATTTTTAATCAATCC
AATATTGAGAATATTGAAAGATAAAACTAAAAAAATAAATACCAAATAAAAAAAATTCAAGTCAATTAGA
AGAACATGTTAAACTCATAACAAGATATTAATATAAAAAATAAATCATAATAAATCACAAAACTTATTTT
TTAATAAATCAAATGTAAAAAAAGAAAAAAAAAAGAAAAACATAGATTAAAAAATTACTGTTGCAATTGC
AATGAATAATAATTTGTAATATATTGCGAAGTAAAAACACCTCCTCTTTAATTTTTTTTTAATATTAATG
TTAATTGGCTGATTTGACATCTTTATTAGCCATTTCCATGTAGAATTCTTTTTTTTTTTTCTGTCTATTT
TGTTCTCATCGATCGAGGAGCATTAACCACCCAAATAAATCTTTTGTTTTAAATTTAATCTCTATATAAT
GTGGCTTGAATCGATGATAAGCACCCCACCAACAAAGATCTTTCAACTCCACATGGCATAAGAGTAGCCT
ATATTTTCTTGGAAAAAATCGACATGGAAATCCACATCCTATCCAGTAACCAATGGGATACCGCTGACGT
GTAGATTTCCTAATCCACAACATCTTGTCATTTCATATACATCTCCCCTCTTCCTGCGTTGATTAGCATC
TACACTCAGTCAGCCTTGATAGAGTAAGAGAAAAGCAGAGCAAGCAACTTAGAGGCAGCATTAACAAAGA
AGAGTC
Sequence ID: 156
Sequence Length: 5170
Sequence Type: DNA
Organism: Poplar sp.
ATATATATTTTTAAAAAAATACTTTAAAAAAAAAACGGATATTGATTGAGTTTAAACAAATGGATGGTAG
TCTTCCAGAGACAAGTACACAATTCCATATATATATTAGCAGCACTTTTCGTGTACGATAGTGATATCTC
ATGCCAGGCCTCGATAAGAGCTCTGTTCATTTTGATCCATACACATTCAAGGGGAATCTCCTTTATAACC
CAAATTGGATGGTGTTAGCAAGTGAATTTGAGCAATGGGCATATAACTTTTCGATAAGATAAACCCTAGC
TAGTTTGCATTTTTGACTTGAATTACAACAAGAAGAAGAAGATGGCGTGCGTACGTGGTGATTCCAATAT
CCCATGCTAGCTAAGATCGTAGTTGTTACAATTCAGTGCAACTCTGATCAGTTCATGTTCAAGATCCAAA
TAATCACTTCAAAGACTAATTCTAAGTGATTACTTTGAATATTAATTATGGATTTGTAATGCTATAAAAT
GTCTGAAGATAGATAGATAGATACTCGTAACTTTAATGTACGTACGTACGTAGACGTTCATGAAGATTCT
CTCTCTGTATATATTTCCAGCAACCCTATAATATTGCTGTAAAATTCTTGCTGGTTCATCTTTACATTTT
AGAACTCGTCTTCCATAATTTGCATGTATTGTGATTAAATCCACTTTTTTCGATTTTTTATGTATAAAAA
AGAAGAAATCAAAGGGAATAATATTACAGATAGAAGCCATCGAGAGAGAATGGCTGGAGAAAATAAAAAA
CTGCATCGATCGCACACTCTGCTGCAACTCAATGCAATAAACAGGAAGACAGCTGCAGATGCTTGATGCC
AGAAACAAAAGTAGAGAAAAGGTTAAATTCAACGAGGAGGGAGGAAACAAATTAATCACCTGGCTCCTTG
AAATCGCCTATCAAATCTTAAAATAAAACATGTATTGAGAAATTCATGCTAACAATTTTTAAGAAAATAG
AAAGTTTCTTGGTAAATTCAATGCTGACAGGAGTCAGGAGCCCTTAATTAGGTTTCGAACCTGGACAGCC
CCGCTCCTGCAAAAGGACTGGGTTTCTACCGATCAGTTTTTCTTAATTTAATAATTTAAATTATTAGATA
AAATTTAAAATATAATTTATATTATTTTTTAATATATGTTTTCAAGTGAAAGCTCTTTGATCTTGAAATT
TACACAGATTTATATTATTTTGTGCTTAATTTTTATCAAATAAATAAAAATGATAAGATTCGAATAACTC
GTGATCGTTTGATTATTAAAATTTTGATAAAATTTCAAGATTAATATATTATTTTTTTAACATGATATGA
TATATATGTAATTCTTTAACATGTAACTTTGAATTAAAGCTTGAAGTGAGAATTGCATATGCATTGAATT
TGGCTTCAAATGTCTTAATTTTTATTCTTTCTGCACTTGTTTAACATGTAACTGAGAATTAGAGCTTGAA
GTGAGAATTGCATATTATGCATTGAATTTGGCTTCAAATGTCTTTTTATTCTTTCTGCACTTTCTTATTT
TAATTTATTCTTTCTGCACTTTCTTATTTTTAACGCGCGTGATAGCATGCAAATTTCCAAATTTATGCAT
CTCGTCTCTTACTTCAAATTGATGCTCTTTCCCAGCTTGGAGAGCTGCCTGGTTCAATTCTACCCACCAA
CTTTGACCAATAAAAGGCAAAAGCTGCAAAAAAAAACATCACATGATAATACCTGTTTTGCCCTTTACAG
AAGAGAAAAAAAAGCTAGTTGGGATATAGGTAGTAATTGTTTTTCAAAGTGTTTTTTATTTTAAAATATA
TTAAAATAATATTTTTTTATTTTTAAAAAATTATTTTTGATATCAGCATATTAAAAAAAATATACAAATA
ACAAAAAATATTAATTTAAAAAAAAATTTAATTTTTTTTTAAAAATACTTTTAAAATACAATACTAAACA
CGCTAACGATAATCTCCTTATTACAAGCTTAGCTATGCACATTATCGACAAGTTGAACATCATGCCAAAT
ATCCAATCTATCATCACCAACTGCTGGTTCAGAAATCCAGATTCCGGCGTCAAAATTCTTGACATGGGTC
AACGATATGCTCCAGTTGCTTACCTGTTTAATGTCAATTTTCTATTAAGACGGTGGGATCCAAATATTTA
ATTACTTGCCAGCAAATGTAATCTGTACTGGTAACGATGGATTGTTTTTGTATGAGTTTTTTTAATTGAA
AATGATTTTTTATATTTTTTTATATTAATATATTAAAATCATTGAAAGTAATTTAAAAATCATCATTTAA
ATTTTTTTAATATAAAAAACACTCTGAAAAATAAATTAAACAACAATAAAAGATTTATTAAATATTTATT
TTTGCATTATAACTTGCTTTTTAAATTAATTTTTAAATTGTTTTTTATTCGAAAAGATATTAAATGGATT
TTTTAGATGTTTTTATATGATTTTAATATATTGATATAAAAAAAATATAATAAAAATTATTTCAATATAT
TTTTAATTAAAAAATATTTTAAAAAAATATTCTACGCTAAAATATTAACTAAATAATATTTAAGCATTCT
ACAGAACATTAACTAACAAACTAAGCAAAGGTTAAACAATTAATATTAATTAATGTGAAATCAACTTGTT
CAAATATTTTATTAGAATAAAGAGGAAAAAATGGTAGAGGGAGGTCATAATTATAGCAAATTTACAGCCC
TTAGACACATACTGGAGCCTCCAACACACCACCCTGTCGATTTTACCTGGAAATTACTCGAGGCTCCCTG
CTTGGCCTCTCGTTTCGTTTATCATGCATGAAAGAAAAATAACCCAATTATTTTAATTCCATAAAATACT
AAAAAGACAAAAGAAAAAACACGACAACCAACTTCCTCGTGTCAAGATCTTGAACCATCTAGTAAACCAA
ACTTGTTCGACCTTTAACACCTGACATGGTATAAAAACCCACTTCCCTTCCCTCTCTTACCCTCACTGCA
TATCGCAATAATTATTAGCGCATCTCCATTGCTACAGGAAGTTGGTGCTCCTCCCCCTATGAAGCAAGCA
TTCCTCCTGGAATGCACTTCTATTGAGTCCGCAAGAAAAAGCAAGGAAAACCCTGCTTGTTAATCATAGT
CTCCAAGCTCTTCAACGCACTTTTAGAAGTCTTCTCAAGACATTTTTTGGTGAGTTCTTTTTCTGTGTAT
TGAGAAACAACGAGAAACTCGGTTGTTTCTATGATCATTCTAGCTAGTTTAATTAGTTTGTCTTGCGCAG
AATATCGAGGAGACATGGTAGCTTCACGGCTAAGATCTTTTAATGTTTGAAGCTTCTTTTTTTTTTTTAG
CTCCTTTTGCTCTCAAATCTTTACCATCAACTTTCCTTTTTGGAATTTTGTTTTGGGTCTTTGGAAGTGG
GATTTTATGTTTCTGAATCTACAGACAAAATTGCTTTAGAGCTAAAAATGAACAAACAGAAAGTGTGAAA
ACATTGGAGCCCCCCCTCTCTACTTCTCTTATTTATTTTTTAATTTTTTCAAGTCCTTAAATCTATTTTA
TGTTTTCACTGAACTCCTTGAATTCTATCTAATTTCCGAACAAGATGCCCACAAAAATCTGAGTGTGATA
TTGTTCTTTTTTCACAGGCACACGAAACTTGAAACGCTGACTAACCCAAAACTCCAAAAACTTAGATTTA
TTTCAGTGTCATATACATTCATCCATTTTTCTAATACTGATTTTAACATTTATTTTTCTGGGAAATACTT
AACAGTCCCTTATATTTGATTCTTTGTATTTTTGAACCAGTTCTTGGAAATTAAGGCTGCAAGAACAATT
GGTGTCTAGTTTGTTTGTTTTTTTTTTTAAAAAAAAAGTGAGAGAAAGGAAGAAAGACATAAATGCTGAG
GATTTTAAGAGACCAACAACAACTGAAGTGGATACCTTGTAATAGTTCGTTGAAGTGGAGGGAGTGGAGA
AGTTACAACAATTGTCGTAGAAACCATCAAACTAGCATCCGCATGAATTACCCTTTTTCCTCGTATTAGC
TTCCGATCCAAAAATCACACAAACCCTTTAGGTCCATATCTAGTAGTCAACCATTTTATACCCTTGGTTT
GGTTTCTTATTATGTTTGTTTTGTGTTTTTTTTTTTTTTGGGTGTGCAGTAACTAAGTGTAAGACAGCAA
CAATGACGGTGACACCAAAGATTTCAATCAATGATGGGAACCTTGTGGTTCATGGGAAGACAATTTTAAC
TGGAGTTCCTGATAACATTGTTCTGACTCCTGGCTCTGGTGTTGGACTTGTTGCTGGTGCTTTTATTGGT
GCTACTGCTTCTCATAACAAAAGTCTCCATGTTTTTCCTGTTGGGGGTTTAGAGTAAGCATTTTGAAGCC
TTTCCTTTCATTGCTGATGATCTTAGGTTATGCGTTTTTTTTCTTTCTTTTGCATTCTTGGAGATGCTCA
CTTTCAATTTTTGTGGCAGGGATCTTCGATTTATGTGTTGCTTCCGATTCAAGTTATGGTGGATGACCCA
GAGAATGGGGAAGTGTGGGAAAGATATTCCACTGGAGACTCAATTCATGCTAGTGGAAAGCAAGGGTGGT
GGTGAAGAAGTTGATCAAGATGATGCACAGACAATCTACACTGTTTTCCTTCCCCTGCTTGAAGGCCAGT
TCCGTGCTGTACTGCAAGGCAACGACAGGAATGAGATGGAGATTTGCCTTGAGAGCGGTGAGTAGATTAT
GGCAATTAAAGTGTCCGGTGCAACGCTAATATGTACCCACCCATGAGCCTAATCTCTCATTATTGAGACC
ACATCACAGACTTATGGGCTGTACCTGTCATTAGTTACCCTACTTCATATGGTCGAAACTACTGTTAATT
CAGTCTCATCCTGTCGGTCACTAGTGAAGGAGACTAAATTCTGAAATTCTGGGTTGGGGCTGTCATTGAT
GTGAACACTACCAGGCTGGAGAACCAATCAATGACTCCACCATCCAGATGAATCGTTTTTTTGGAGTTCC
TTTCATTTGGAAGAAATAGATAGAGAGGATCGAGTTTGAAAGAAACCGAAACGATGCGTTTTTTTGTGGT
TGATTTGGTTTTGCAGGCGATAGTGCTGTTGAAACCAACCAAGGCCTTCACCTGGTCTAC
Sequence ID: 157
Sequence Length: 3000
Sequence Type: DNA
Organism: Poplar sp.
GTGAATCAAGGTTGTTTAAATCATAATTTAACCCATAAAAATTATATAATTTTACGAGTTAATATATGTT
TAACATGCAGAATCGTATTAAAAACTCGAAAATTAATAAAATCGGGTTGAAATCGAGTGAAATCGGTAAA
ATTAAATGAAATCACAATATATATTTTTAAGATACTTGAACTATATATGATTTTTTATTTGAATGATGTA
TTTTAAGATGTTTGAAAAAAAATTTTCTTATAATTTTATGATTTTACAATCTATTTTTGTGATTCAAGTT
TATTTTATATTTTTTATGTTATTTTAAAAATATATTTTTAAAAACCTTGATGTGGACAGTACACCAGTAG
TAGCTGACAAGATTTTAGTCTATTTTAAAAATCCCGTATTTTATAAAAGAAAAACACACAATCATTAACG
AGTATGAGTGAACAATCAGCAGCCCAAGCATCCTCCTCTGCTTGCCAAAAATTGTTCCTTTTTTAAAAGA
CACGTCTTTGTTCCAAAAACAATATCGTAGTATATTAATAATTCGCCAAAATAATTAACGTTATATTCTC
TCGGTGGGCCCCGGATTGGTTGTGAAACACGATGATTAATGGGCCTCTTCTCTTGACAGGATAACAATGA
TTTAGGATATCATGTGAGTAACAGTTATTTTTTATTTTAAAACATATTAAAATAATTAAAATATAATTTA
AAGTAAAAAAATTCTTTAATTTTTTTTTGAAATACAAAAATAATTTTGCTGTGAAAGTGAGACGGTGACA
ATATAAGGATTTTAGAAGTTTAATTACGGTGTAGAATAATCATTTCAACTTAATAATTTAAATTGTTAAG
TAAGATATTAAGATATAATTTATATTATTTTATAGTATATCCTCCCAAAGAAAATATTTTTAAATTTAGA
AATTTACACAGATTAATATTATTTTATATTTAATTTTTATCAAATAAATAAAAATGATGAGATTCGAAGT
AGTAATGGTTTGATCATTTAAATTCTCATATAAAAAACACCTCATTTTAATAACTAAAATTATTAAATAA
AATTTTAAAATATAATCTATACTGTTCAAGGCTACACGATGTCATTAATTTTATAGACGCTGGTCCAAAT
CAGTAATGCATACACTTTGTACGCAAGCAAAGCAACCAAACTAGAGAGAGGAAAGGAGGAGAGGGCAGTA
GCGTGGAAAGGGAGGGAAGCATAGCTTAAAGTGTCGGCAAACGTTCTGGTTGAAACTTCTCGAAGAGAAA
AGGAGAGAGAGGGTAACTGGTGGTTAGATAGAGGAGGAGGATGCCCCCATCAAAAACTATACACTCTCTC
TTGCAGATTGCTCCTCCAAGAATGAAAACATGCTCAAAATCACCATCAGTTTTGTATATGTGTAGTTAAT
TATGCATCTTCCTGTAGTTTATCAACAATTTACACAGCTTGAGGGGACAAAATTAACTTGTTGATGTGCA
TAATCGATAATTCCTTCTAATCCATGTAAATCAAACGGGTTACAGTGATCCATGTACAATATATTTTTTT
TTAGTTTAACGTGGGTGTCCGGGTCAGCTTGCGCGCACCTCGATTAATTTCATGGACCCTGAAGTTAACG
ACCATGTAAACCTCTAGTGGTCATCATATGAGCAATTACAGGGCTCGAACCTGAGACTACAGAGGAAACA
AATCTCTTGGTCCCAAGCTTTTACCACTGGCCCACCACTAGATGGTTGATCCATGTATAATATTGTATGT
ACACGTTCTTGTTTATCTATTTTCGAATATAGCTGAGATTTAATTTTTAGAAATTACTACTTCAAGTATT
ATAAATCTCGGATCATTAAAAGTTTATAATTATTATATAATTATTAAATTTAAAATTTTAAAAAATTAAT
TAAAATACATGTAAATTAATCTAATACTACAATTAAAAATAAAAAAAAATGATTTGTCCGCTTTGGAAAG
TGAAAGGGATGAGGTTCCTTACCATCGTTAGTTGGATGGCATCTTTTCATTCACCAACACTAAAAAATTA
CGATCAAAACCAACCCCACTTTACAAGCAATTATGATGTCTCCTCTCTTCCCCAAAACTGGAGGTGCTAA
AGACCATGGAAAATTAATTGTAGTAAATTATTTTTAAAATGTTTTTGGTTTTGAAATTATATTAAAATAA
TATTTTTTATTTTTAATATTAGTTATATTAAAATATTAATTTAAAAAATTAAAATTCAAAATTTTTAAAA
ATCCAACTTCAAAAGTAGGTGAAGCCCTAGTCTTAATTAATATGTCAGTTTGTTTTTATATTTAAAAAAT
ATTTTTAACAGAATTTAAATTTTATTTATTAAAATGAACAAAAAATTATTTTAATATATTAAAAAAATAT
TTTAAAAAATAATGGGATTAATAAACACCATGTGAACGGGTTGAAGTTCTTGGAAAGCAAATTCCATTAC
CAAATTCTACTTTTCTTCTTCTTCCTTTTTTTTTTTGGGCCCACTGTCTAGAGTGAGAAAGAAAGAAATA
CACCGCAGATTTAGCTGTTAGAAACACAGCGGTGGACAGACAGGCTGTCCTTGTCTAGTAGCGGACCGGT
CACCCTCACAACCACATTCAATAAATTCAATAAATATAATTCCGTACTTAAAAATGTTAGCAAAAACGTG
GAATAAAAATAAAAATAAAAACCCCAACCCAACCCAAGTTTCTCACATGATATGATACTGCTTGTTGCTT
GACTCTCCACCGTCATCAGTCTCCTCTGTCCACCATTCATAATAGCCTCCCGCATCCCCACGCTCCATCA
CTCCTCCCCACACTTGAGAAAGAGAGAGAGTAACACAGCTCGCATTTTCTTTTTGTCAATACAGTGTCGG
TGGAGCTTCGAGTTCTCTGCCTTTTTGTTTTTGTTTTCGTTTTCGTTTTCGTTTTCTTGAATTGAGGGAG
GATCAAAAGTAAGGTAATTTACTAAATTAATACAAGAAGAAAGGGTGAAATCTTAAGAAT
Sequence ID: 158
Sequence Length: 1815
Sequence Type: DNA
Organism: Poplar sp.
AGATTCTTCATAGAGGGAGCAAGAAAATAATTATTTTGTAAATGTATAGCTAGAAATTAAGTGGTATCATTCGT
GAAGCTTTATCTAATACTTTAATAAGATTTAGATTTAGAATTTTTATTTAGAATTGGGACAGTTCATTGATATT
GGATAGATTTTTTATGATAAAGAATAATGAATTCTACTAGTATCATCTTCTTGATTTTTTAATTAAAATTAAAT
CATCAGAGGATAATAAGGGATTATGCAAGGTTTAATTTAGTGTAAAAAAGTTTTTTATTTGAAGGAAAGTGATA
AGTATTAATATAAAATTAGTCACCAAAAAAACATAAACTATGAGCTGAGATATTTAGTTTTGACGGAAGAAAAC
TTCATGCCTAAACTGTAATTATTTGAACAAGTATTGAGATAAGTTGTGGGCGTCCGTCGTGATTTATATATCAA
TTAGGATGAGTTGAAGAGTAGATAATGAGAGGAAAAACAAACTAGTAATTAATCACATGACTGCTACAACAACC
AAGCCCACTAATTAAAGGGCTTCTTCGTGGTAGGTACGTATTTCGCATGCAGGAGGCAAGACTCGTTCAATTAA
TAATTATAAAAAAAATAATTAGGAGTGTAACTCAACTGGTCAGGTTTTAAGTTTGTATTTTAGATATTATTAAT
TTGATTCTCTTAAACTTCAGGACCACTAGAGACTTATATGATCATTAACTTTAGGGCTCATGAAATTAGTCGAG
ATATACTCAAACTAACTTTAAGACTTATGTTAATAGAAAAATAAAAATAAAAGAAGTAACTATACAACCACCAA
CATGATGTTGTATATACCGAACATTGCTTTGCTTAAGTAGCCTGTTTAGAATAATATGTAATTAATTATATATG
TTGGTGGAAACGATCAATTACACCTCCTTTTTATTTATGAGATTCCCTTAATTTTACCTGGCTTGAATCCGGCA
GTACTAATTAAATACTAGCCATGTTGTTGATGAACATGCATCATCAGTATACTTCCCTCCCCAACCTCTATATA
TTGCTTTGTTTTTTCTTAAATAGCAATATTTCGTTAGCTGGGAATTAGTCGCACGTTTATTTTAGGTTGCTGAC
TGATTCTAGGTAATAATATCATCACACGGCATGGACAATAGTTATGTCTGACTTGAATTAAATATCCAAGAGAT
AGATAATTACCTCATTATTTGTTGGGGTGTAAGAGAATTTCACTCGTGAGAATTACCGATGATCATGACGACAT
GGGAGCACAAGTCAAGGATACATGCCATTCGGTGAAGAAAAATGCATGATTATTAGGTGGATCCATATGGTATA
CTATTTGCTTTACAGCCCCACGAGACTAATATACTTTATGGACACCGATATTATTCATTTTACATGGCAAGACG
AAGTTTCCCGATTCTCACCAACCACAACCTCACCACCATGCACCACTCCCACGAGTCATATTTACCCTTCTAAG
CCAATCGCCAATATTGATCCACGAAGACTCCACGCTCCATGGTTTGTACCCTTAGATTGTTCCAAATCAATGGC
CACAATTCAGTCTCAACAAACCCACTTTGTCACCTCTTGCCGGCCCTCATGCTTACCTACCATCTCCTGTGCAT
GATATTCAAGACTCCCAAGTCCCAACCCTCTATTTAAACCCCCTACACCTCCCCTCTATTTCTCTCAGGAAATC
CCGTACTCCAGAACTCAAGATTTCCTCTACAGCTACTCCTCTTCCTCCATTCTTCAAGACTCCTTCTCTCTCAT
TTAAGCTTTTCTTTGGTCTAACATATATATAAATAAGAA

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. An isolated nucleic acid whose nucleotide sequence comprises a sequence having at least 85% identity to at least one of SEQ ID NO: 1 to 158.

2. The isolated nucleic acid of claim 1, wherein the nucleic acid regulates gene expression when operably linked to a gene.

3. The isolated nucleic acid of claim 1, wherein the nucleic acid has a nucleotide sequence comprising a sequence having at least 85% identity to at least one of SEQ ID NO:90, 94, 103, 117, 131, 137, 145, and 158.

4. The isolated nucleic acid of claim 1, wherein the nucleic acid has a nucleotide sequence comprising a sequence having at least 85% identity to at least one of SEQ ID NO: 90 and 103.

5. A vector comprising a gene regulatory element whose nucleotide sequence has at least 85% identity to at least one of SEQ ID NO: 1 to 158.

6. The vector of claim 5, wherein the gene regulatory element has a nucleotide sequence having at least 85% identity to at least one of SEQ ID NO: 90, 94, 103, 117, 131, 137, 145, and 158.

7. The vector of claim 5, wherein the gene regulatory element has a nucleotide sequence having at least 85% identity to at least one of SEQ ID NO: 90 and 103.

8. The vector of claim 5, further comprising a heterologous gene operably linked to the gene regulatory element.

9. The vector of claim 8, wherein the gene regulatory element regulates expression of the heterologous gene.

10. The vector of claim 8, further comprising a gene terminator sequence operably linked to the heterologous gene.

11. The vector of claim 8, wherein the heterologous gene encodes an enzyme polypeptide.

12. The vector of claim 11, wherein the enzyme polypeptide is a cell wall modifying enzyme polypeptide.

13. The vector of claim 12, wherein the cell wall modifying enzyme polypeptide is of an origin selected from the group consisting of archaeal, fungal, insect, animal, and plant.

14. The vector of claim 11, wherein the enzyme polypeptide is a lignocellulolytic enzyme polypeptide.

15. The vector of claim 8, wherein the heterologous gene encodes a polypeptide selected from the group consisting of stress resistance polypeptides, nutrient utilization polypeptides, mycotoxin reduction polypeptides, and male sterility polypeptides.

16. The vector of claim 8, wherein the heterologous gene encodes a polypeptide that confers resistance to at least one herbicide.

17. The vector of claim 16, wherein the heterologous gene encodes a polypeptide selected from the group consisting of phosphinothricin acetyltransferase, glyphosate-resistant enolpyruvoyl-shikimate-3-phosphate synthetase, dalapon dehalogenease, and bromoxynil nitrilase.

18. The vector of claim 8, wherein the heterologous gene encodes a polypeptide that confers resistance to infestation from at least one organism.

19. The vector of claim 18, wherein the polypeptide confers resistance to infestation from an organism selected from the group consisting of insects, bacteria, fungi, and nematodes.

20. The vector of claim 8, wherein the heterologous gene encodes a polypeptide that confers resistance to at least one virus.

21. The vector of claim 8, wherein the heterologous gene encodes an RNA molecule that regulates a plant gene.

22. The vector of claim 8, wherein the heterologous gene encodes a polypeptide having therapeutic value.

23. The vector of claim 8, wherein the heterologous gene encodes a polypeptide selected from the group consisting of phosphomannose isomerase and anthranilate synthase.

24. The vector of claim 5, further comprising a selectable marker gene.

25. The vector of claim 23, wherein the selectable marker gene encodes aminoglycoside phosphotransferase, hygromycin phosphotransferase or neomycin phosophotransferase.

26. The vector of claim 5, wherein the vector is a binary vector.

27. The vector of claim 5, wherein the vector is an expression vector.

28. The vector of claim 5, wherein the vector is a plasmid.

29. A transgenic plant, the genome of which is augmented with:
a recombinant polynucleotide comprising a gene regulatory element that has at least 85% nucleotide sequence identity to at least one of SEQ ID NO: 1 to 158.

30. The transgenic plant of claim 29, wherein the gene regulatory element has at least 85% sequence identity to at least one of SEQ ID NO: 90, 94, 103, 117, 131, 137, 145, and 158.

31. The transgenic plant of claim 30, wherein the gene regulatory element has at least 85% sequence identity to at least one of SEQ ID NO: 90 and 103.

32. The transgenic plant of claim 29, wherein the recombinant polynucleotide further comprises a heterologous gene operably linked to the gene regulatory element.

33. The transgenic plant of claim 32, wherein the gene regulatory element regulates expression of the heterologous gene.

34. The transgenic plant of claim 32, wherein the heterologous gene encodes an enzyme polypeptide.

35. The transgenic plant of claim 34, wherein the enzyme polypeptide is a cell wall modifying enzyme polypeptide.

36. The transgenic plant of claim 35, wherein the cell wall modifying enzyme polypeptide is of an origin selected from the group consisting of archaeal, fungal, insect, animal, and plant.

37. The transgenic plant of claim 34, wherein the enzyme polypeptide is a lignocellulolytic enzyme polypeptide.

38. The transgenic plant of claim 32, wherein the heterologous gene encodes a polypeptide selected from the group consisting of stress resistance polypeptides, nutrient utilization polypeptides, mycotoxin reduction polypeptides, and male sterility polypeptides.

39. The transgenic plant of claim 32, wherein the heterologous gene encodes a polypeptide that confers resistance to at least one herbicide.

40. The transgenic plant of claim 32, wherein the heterologous gene encodes a polypeptide selected from the group consisting of phosphinothricin acetyltransferase, glyphosate-resistant enolpyruvoyl-shikimate-3-phosphate synthetase, dalapon dehalogenease, and bromoxynil nitrilase.

41. The transgenic plant of claim 32, wherein the heterologous gene encodes a polypeptide that confers resistance to infestation from at least one organism.

42. The transgenic plant of claim 41, wherein the polypeptide confers resistance to infestation from an organism selected from the group consisting of insects, bacteria, fungi, and nematodes.

43. The transgenic plant of claim 32, wherein the heterologous gene encodes a polypeptide that confers resistance to at least one virus.

44. The transgenic plant of claim 32, wherein the heterologous gene encodes an RNA molecule that regulates a plant gene.

45. The transgenic plant of claim 32, wherein the heterologous gene encodes a polypeptide having therapeutic value.

46. The transgenic plant of claim 32, wherein the heterologous gene encodes a polypeptide selected from the group consisting of phosphinothricin acetyltransferase, phosphomannose isomerase, glyophosphate resistant 5-enolpyruvoyl-shikimate-3-phosphate synthetase (EPSPS), aminoglycoside phosphotransferase, dalapon dehalogenease, bromoxynil resistant nitrilase, and anthranilate synthase.

47. The transgenic plant of claim 32, wherein the recombinant polynucleotide further comprises a gene terminator sequence operably linked to the heterologous gene.

48. The vector of claim 32, wherein the heterologous gene encodes a polypeptide selected from the group consisting of phosphomannose isomerase and anthranilate synthase.

49. The transgenic plant of claim 29, wherein recombinant polynucleotide further comprises a selectable marker gene.

50. The transgenic plant of claim 49, wherein the selectable marker gene encodes aminoglycoside phosphotransferase, hygromycin phosphotransferase or neomycin phosphotransferase.

51. The transgenic plant of claim 29, wherein the plant is fertile.

52. The transgenic plant of claim 29, wherein the plant is not fertile.

53. The transgenic plant of claim 29, wherein the plant is a monocotyledonous plant.

54. The transgenic plant of claim 53, wherein the monocotyledonous plant is selected from the group consisting of bamboo, barley, maize (corn), millet, miscanthus, rice, rye, sorghum, sugarcane, switchgrass, turfgrass, and wheat.

55. The transgenic plant of claim 29, wherein the plant is a dicotyledonous plant.

56. The transgenic plant of claim 55, wherein the dicotyledonous plant is selected from the group consisting of alfalfa, Arabidopsis, aspen, birch, eucalyptus, flax, canola, cotton, cottonwood (e.g., Populus deltoides), hemlock, hemp, larch, oil seed rape, potato, poplar, sisal, spruce, soybean, sunflower, sweetgum, tobacco, tomato, and willow.

57. A transformed seed from the transgenic plant of claim 29.
